(19) United States
(12) Patent Application Publication
(10) Pub. No.: US 2024/0299385 A1
(43) Pub. Date: Sep. 12, 2024

(54) 3-PYRROLYLSULFONAMIDE COMPOUNDS AS GPR17 ANTAGONISTS

(71) Applicant: ReWind Therapeutics NV, Heverlee (BE)

(72) Inventors: Vincent Pericolle, Leuven (BE); Jean-Christophe Vanherck, Heverlee (BE); Arnaud Didier Marie Marchand, Bierbeek (BE); Guillaume Albert Jacques Duvey, Thyez (FR); Rui Miguel Garcia Costa Pinto, Leuven (BE)

(73) Assignee: ReWind Therapeutics NV, Heverlee (BE)

(21) Appl. No.: 18/561,189

(22) PCT Filed: Jun. 3, 2022
(86) PCT No.: PCT/EP2022/065235

§ 371 Date: Nov. 15, 2023

(30) Foreign Application Priority Data
Jun. 4, 2021 (EP) 21177731.3

(51) Int. Cl. A61K 31/506; A61K 31/4025; A61K 31/4178; A61K 31/4184; A61K 31/422; A61K 31/4245; A61K 31/427; A61K 31/437; A61K 31/4439; A61K 31/444; A61K 31/4709; A61K 31/497; A61K 31/501; A61P 25/28; C07D 207/36; C07D 401/04; C07D 401/12; C07D 401/14; C07D 403/04; C07D 405/04; C07D 405/12; C07D 409/04; C07D 409/06; C07D 413/04; C07D 413/12; C07D 417/04; C07D 417/12; C07D 471/04

(52) U.S. Cl. CPC A61K 31/506; A61K 31/4025; A61K 31/4178; A61K 31/4184; A61K 31/422; A61K 31/4245; A61K 31/427; A61K 31/437; A61K 31/4439; A61K 31/444; A61K 31/4709; A61K 31/497; A61K 31/501; A61P 25/28; C07D 207/36; C07D 401/04; C07D 401/12; C07D 401/14; C07D 403/04; C07D 405/04; C07D 405/12; C07D 409/04; C07D 409/06; C07D 413/04; C07D 413/12; C07D 417/04; C07D 417/12; C07D 471/04

(57) ABSTRACT

The present invention relates to a compound of formula (I), or a tautomer, a stereoisomer, a hydrate, a solvate, a polymorph, a prodrug, an isotope, or a co-crystal thereof, or a pharmaceutically acceptable salt thereof, wherein R¹, R², R³ and R⁴ are as defined in the description and claims. The present invention also relates to a pharmaceutical composition comprising a compound according to the invention, and a pharmaceutical acceptable carrier. The present invention also relates to the present compounds for use as a medicine and/or as diagnostics. The present invention also relates to the present compounds for use in the prevention and/or treatment of GPR17 mediated disorders, such as for example a disorder or syndrome selected from a myelination disorder and a disorder or syndrome associated with brain tissue damage.

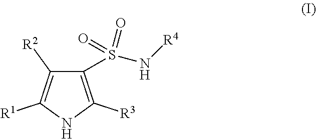

### FIELD OF THE INVENTION

The present invention relates to new pyrrolyl-sulfonamide compounds and their use for treating and/or preventing GPR17 mediated disorders. The present invention also relates to said compounds for use as a medicine and/or in diagnostic methods, more preferably for use as a medicine to treat and/or prevent GPR17 mediated disorders. The present invention furthermore relates to pharmaceutical compositions or combination preparations of the compounds, to the compositions or preparations for use as a medicine and/or in diagnostic methods, more preferably for the prevention and/or treatment of GPR17 mediated disorders. The invention also relates to processes for preparation of said compounds.

### BACKGROUND OF THE INVENTION

GPR17 is a member of a class of membrane receptors called G-protein coupled receptors (GPCRs). These receptors are characterized by a seven transmembrane domain structure with an intracellular region that couples through G proteins to numerous of intracellular signaling pathways. Many GPCRs have been used as targets for pharmaceutical drugs and diagnostics.

GPR17 is currently considered an orphan GPCR, reflecting the fact that the endogenous ligand(s) for the receptor has not been conclusively identified. The expression of GPR17 has been identified in the central nervous system (CNS) but also outside the CNS (Lecca et al., Glia. 2020 October; 68(10):1957-1967) in various human organs, such as heart and kidney, i.e., organs typically undergoing ischemic damage. There are two forms of the receptor that are expressed in humans which vary in the inclusion of a 28 amino acid sequence on the N terminal. The short form of the receptor lacking the 28 amino acids is generally thought to be expressed in the CNS, while the long form of the receptor is expressed outside the CNS, e.g., in the heart and the kidney (Benned-Jensen and Rosenkilde, Br J Pharmacol. 2010 March; 159(5): 1092-1105). The sequence of the receptor is largely conserved between species, and the rodent and human forms of the receptor are about 90% identical. As such, experiments that use mice or rats to study GPR17 are expected to reflect the characteristics of GPR17 in humans.

Although the endogenous ligand(s) for GPR17 has not been conclusively identified, it has been possible to study the properties of the receptor by inducing its expression in different cell lines, including HEK293 and CHO cells. Using these expression systems, activators and inhibitors of the receptor have been identified. Activators include the compound MDL 29,951 (Hennen et al., Sci Signal. 2013 Oct. 22; 6(298): ra93). Inhibitors include the compounds pranlukast and HAM13379 (Simon et al., Mol Pharmacol. 2017 May; 91(5):518-532; Merten et al., Cell Chem Biol. 2018 Jun. 21; 25(6):775-786). These compounds are useful tools to study the signaling properties of GPR17, but their utility is limited by a lack of selectivity for GPR17. For example, MDL29,951 is approximately ten-fold more potent as an NMDA receptor antagonist than as a GPR17 activator, and pranlukast is approximately 1,000-fold more potent as an inhibitor of cysteinyl leukotriene receptors.

Effective modulation of the GPR17 activity may have neuroprotective, anti-inflammatory, and anti-ischemic effects and may thus be useful for the treatment of cerebral, cardiac, and renal ischemia, and stroke, and/or for improving the recovery from these events (Bonfanti et al, Cell Death Dis. 2017 June; 8(6): e2871). Moreover, pulmonary fibrosis may be alleviated through suppressing GPR17-mediated inflammation (Zhan et al., Int Immunopharmacol. 2018 September; 62:261-269). GPR17 modulators are also thought to be involved in food uptake, insulin and leptin responses and are thus could have a role in obesity treatment (Ren et al., Cell. 2012 Jun. 8; 149(6): 1314-1326).

The function of GPR17 in the CNS can be illustrated by experiments where the receptor is removed or overexpressed in mice (Chen et al., Nat Neurosci. 2009 November, 12(11):1398-406). Mice overexpressing GPR17 show a deficit in the production of myelin, which is the sheath formed around axons by oligodendrocytes, and which is necessary for the maintenance of signal transduction and neuronal function. As a result of the deficit in myelin production, GPR17 overexpressing mice die within one month of birth. Conversely, mice in which GPR17 is knocked out show precocious myelination. These findings suggest that GPR17 plays an important role in controlling myelin production. This conclusion is consistent with the observation in rodents and humans that GPR17 is selectively expressed in oligodendrocyte precursor cells (OPCs). OPCs are stem cells that are found in the brain throughout life. OPCs differentiate into oligodendrocytes which are then able to form myelin. The selective expression of GPR17 in OPCs and the observations in mice in which GPR17 expression is modulated is consistent with the conclusion that GPR17 regulates the formation of myelin (Lecca et al., Glia. 2020 October; 68(10):1957-1967). Moreover, these findings also suggest that decreasing the activity of GPR17 with antagonistic or inverse agonistic compounds will increase myelin formation. This conclusion is supported by numerous additional findings, including observations that GPR17−/− mice have enhanced remyelination following a toxin-induced injury compared to littermate controls (Ou et al., J Neurosci. 2016 Oct. 12; 36(41):10560-10573), and also from findings that selective antagonists of GPR17 enhance remyelination following cuprizone-induced demyelination.

Myelin is an essential component of a healthy CNS. The failure to form myelin, damage to myelin and/or the failure to repair myelin may cause certain diseases and may also be a secondary consequence of certain diseases. One example of a disease that is primarily a result of damage to myelin is multiple sclerosis (MS). The cause of MS is not known, but it affects approximately 400,000 people in the United States and about 2.5 million people worldwide and is approximately three times more likely to occur in women than men. MS is an inflammatory autoimmune disease that arises from an immune attack directed at oligodendrocytes which results in myelin damage and ultimately loss of neuronal axons. The immediate consequence is a collection of acute symptoms that include difficulty in movement, speech, swallowing, dizziness, and fatigue. Symptoms may also include problems with vision, hearing, or balance. The disease can take several forms. One form is associated with relapses and remissions where the acute symptoms resolve overtime, and this form is termed relapsing remitting multiple sclerosis (RRMS). Another form of the disease, primary progressive MS (PPMS) is characterized by a failure to resolve symptoms between attacks, and is considered a more severe form of the disease. In most forms of MS there is a progressive accumulation of symptoms that do not resolve, and this results in an increasing burden of disability. There are a number of treatments for MS that have received regulatory approval. These treatments have an effect on the frequency of relapses but are much less effective on the progression of disability. It has been proposed that compounds that promote the differentiation of OPCs and thus the formation of new oligodendrocytes will be effective in treating the progression of disability in MS by promoting the repair process (Lubetzki et al., Lancet Neurol 2020; 19: 678-88).

A number of other CNS diseases are associated with abnormal function of myelin. Acute injury such as ischemic brain injury or traumatic brain injury results in damage to myelin (Lecca et al., PLoS One. 2008; 3(10):e3579; Shi et al., Exp Neurol. 2015 October; 272:17-25). There are a number of diseases of myelin deficiency that result from inherited mutations or toxin exposure (Duncan and Radcliff, Exp Neural. 2016 September; 283 (Pt B):452-75). In other diseases, such as Alzheimer's disease the loss of brain volume that accompanies the progression of the disease is partially attributable to the loss of oligodendrocytes and myelin (Chacon de la Rocha et al., Front Cell Neurosci. 2020 Dec. 3; 14:575082). More subtle forms of myelin dysfunction may be associated with diseases such as schizophrenia and autism, where the failure to form fully mature myelin may contribute to the cause or the symptoms of the disease (Marie et al., PNAS Aug. 28, 2018, 115 (35) E8246-E8255; McPhie et al., Translational Psychiatry 2018. 8:230). In each of these cases, promoting the formation of mature and fully functional myelin may have an important therapeutic effect. As a key regulator of OPC maturation, GPR17 antagonists may thus be valuable for the treatment of a wide range of diseases.

There is no known causal treatment or cure for multiple sclerosis, or many other myelination diseases. Treatments are usually symptomatic and try to improve function after an attack and prevent new attacks, by addressing the inflammatory component of the disease. Such immunomodulatory drugs are usually only modestly effective, in particular if the disease is progressed, but can have side effects and be poorly tolerated. Moreover, most of the available drugs, like β-interferons, glatiramer acetate, or therapeutic antibodies are only available in injectable form and/or only address the inflammatory component of the disease but not demyelination directly. Other drugs, like corticosteroids, show rather unspecific anti-inflammatory and immunosuppressive effects thus potentially leading to chronic side effects, such as manifested in Cushing's syndrome, for example.

There is clearly a need for a safe and effective drug for the treatment of GPR17 mediated diseases such as myelination diseases, like MS, preferably for a drug that is suitable for oral administration. Ideally such a drug would reverse the demyelination process by decreasing demyelination and/or by promoting remyelination of the impacted neurons. A chemical compound which effectively decreases the GPR17 receptor activity could fulfil these requirements.

There is therefore a need for GPR17 modulators, preferably negative GPR17 modulators, which are capable of effectively decreasing the GPR17 activity.

### SUMMARY OF THE INVENTION

The present invention is based on the unexpected finding that the below described new class of pyrrolyl-sulfonamide compounds are negative modulators of GPR17.

In particular, in a first aspect, the present invention provides a compound of formula (I), or an isomer (such as a tautomer or a stereoisomer), a hydrate, a solvate, a polymorph, a prodrug, an isotope, or a co-crystal thereof, or a pharmaceutically acceptable salt thereof, as defined in the appended claims and description,

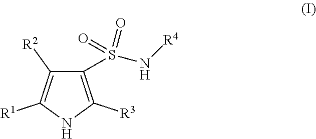

wherein H (I)
R¹ is selected from the group comprising aryl, heteroaryl, cycloalkyl, cycloalkenyl, cycloalkynyl, heterocyclyl, and A¹-X¹—; and R² is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl;
   wherein each of said aryl, heteroaryl, cycloalkyl, cycloalkenyl, cycloalkynyl, heterocyclyl, X¹ and A¹ of R¹ can be unsubstituted or substituted with one or more Z¹;
X¹ is —Y^{1b}—Y^{1a}—Y^{1c}— wherein Y^{1a} is a single bond, double bond or triple bond or is selected from the group comprising —CR^{1a}═CR^{1a}—, —C≡C—, —CO—, —O—, —CS—, —S—, —SO₂—, —SO—, —SO(NH)—, —CONR^{1b}—, —NR^{1b}CO—, —SO₂NR^{1b}—, —NR^{1b}SO₂—, —S(O)—NR^{1b}—, and —NR^{1b}—;
each of Y^{1b} and Y^{1c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{1a}; wherein when Y^{1a} is a single bond, double bond, or triple bond, at least one of Y^{1b} and Y^{1c} is not a single bond;
each R^{1a} is independently selected from the group comprising hydrogen, oxo, thioxo, halo, hydroxy, haloalkyl, alkoxy, alkoxyalkyl, haloalkoxy, haloalkoxyalkyl, mono or di(alkyl)amino, mono or di(alkyl)aminoalkyl, and alkyl;
A¹ is selected from the group comprising aryl, heteroaryl, cycloalkyl, cycloalkenyl, cycloalkynyl, and heterocyclyl;
each Z¹ is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, hydroxyalkyl, alkoxyalkyl, cycloalkyloxy, cycloalkylalkoxy, alkoxyalkoxy, carboxyl, alkoxycarbonyl, alkylcarbonyl, arylalkoxy, amino, mono or di(alkyl)amino, aminoalkyl, mono or di(alkyl)aminoalkyl, mono or di(alkyl)aminocarbonyl, heterocyclyl, heteroaryl, heterocyclylalkyl, heteroarylalkyl, arylalkenyl, arylalkynyl, haloalkenyloxy, haloalkynyloxy, hydroxyalkenyl, hydroxyalkynyl, alkenyloxyalkyl, alkynyloxyalkyl, alkoxyalkenyl, alkoxyalkynyl, alkenyloxyalkoxy, alkynyloxyalkoxy, alkenyloxycarbonyl, alkynyloxycarbonyl, alkenylcarbonyl, alkynylcarbonyl, aminoalkenyl, aminoalkynyl, mono or di(alkyl)aminoalkenyl, mono or di(alkyl)aminoalkynyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{1a};
and/or two Z¹ together with the atom(s) to which they are attached can form an aryl, a cycloalkyl, a heteroaryl, or a heterocyclyl; wherein each of said aryl, cycloalkyl, heteroaryl, and heterocyclyl can be unsubstituted or substituted with one or more Z^{1a};
and/or one R^{1a} together with one Z¹ and the atom(s) to which they are attached can form a cycloalkyl, a 4-10 membered saturated or partially saturated heterocyclyl, a 5-10 membered heteroaryl, or an aryl; wherein each of said cycloalkyl, heterocyclyl, heteroaryl or aryl can be unsubstituted or substituted with one or more Z^{1a};
R^{1b} is hydrogen or alkyl, or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a};
each Z^{1a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
or R¹ is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl; and R² is selected from the group comprising aryl, heteroaryl, cycloalkyl, cycloalkenyl, cycloalkynyl, heterocyclyl, and A²-X²—;
   wherein each of said aryl, heteroaryl, cycloalkyl, cycloalkenyl, cycloalkynyl, heterocyclyl, X² and A² of R², can be unsubstituted or substituted with one or more Z²;
X² is —Y^{2b}—Y^{2a}—Y^{2c}—, wherein Y^{2a} is a single bond, double bond or triple bond or is selected from the group comprising —CR^{2a}═CR^{2a}—, —C≡C—, —CO—, —O—, —CS—, —S—, —SO₂—, —SO—, —SO(NH)—, —CONR^{2b}—, —NR^{2b}CO—, —SO₂NR^{2b}—, —NR^{2b}SO₂—, —S(O)—NR^{2b}—, and —NR^{2b}—;
each of Y^{2b} and Y^{2c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{2a}; wherein when Y^{2a} is a single bond, double bond, or triple bond, at least one of Y^{2b} and Y^{2c} is not a single bond;
each R^{2a} is independently selected from the group comprising hydrogen, oxo, thioxo, halo, hydroxy, haloalkyl, alkoxy, alkoxyalkyl, haloalkoxy, haloalkoxyalkyl, mono or di(alkyl)amino, mono or di(alkyl)aminoalkyl, and alkyl;
A² is selected from the group comprising aryl, heteroaryl, cycloalkyl, cycloalkenyl, cycloalkynyl, and heterocyclyl;
each Z² is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, arylalkenyl, arylalkynyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, haloalkenyloxy, haloalkynyloxy, hydroxyalkyl, hydroxyalkenyl, hydroxyalkynyl, alkoxyalkyl, alkenyloxyalkyl, alkynyloxyalkyl, alkoxyalkenyl, alkoxyalkynyl, cycloalkyloxy, cycloalkylalkoxy, alkoxyalkoxy, alkenyloxyalkoxy, alkynyloxyalkoxy, carboxyl, alkoxycarbonyl, alkenyloxycarbonyl, alkynyloxycarbonyl, alkylcarbonyl, alkenylcarbonyl, alkynylcarbonyl, arylalkoxy, amino, mono or di(alkyl)amino, aminoalkyl, aminoalkenyl, aminoalkynyl, mono or di(alkyl)aminoalkyl, mono or di(alkyl)aminoalkenyl, mono or di(alkyl)aminoalkynyl, mono or di(alkyl)aminocarbonyl, heterocyclyl, heteroaryl, heterocyclylalkyl, heteroarylalkyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, arylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{2a};
and/or two Z² together with the atom(s) to which they are attached can form an aryl, a cycloalkyl, a heteroaryl, or a heterocyclyl; wherein each of said aryl, cycloalkyl, heteroaryl, and heterocyclyl can be unsubstituted or substituted with one or more Z^{2a};
and/or one R^{2a} together with one Z² and the atom(s) to which they are attached can form a cycloalkyl, a 4-10 membered saturated or partially saturated heterocyclyl, a 5-10 membered heteroaryl, or an aryl; wherein each of said cycloalkyl, heterocyclyl, heteroaryl, or aryl can be unsubstituted or substituted with one or more Z^{2a};
R^{2b} is hydrogen or alkyl, or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};
each Z^{2a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
R³ is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl;
R⁴ is aryl, or heteroaryl; wherein each of said aryl and heteroaryl, is substituted with one or more Z⁴;
each Z⁴ is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, arylalkenyl, arylalkynyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, haloalkenyloxy, haloalkynyloxy, hydroxyalkyl, hydroxyalkenyl, hydroxyalkynyl, alkoxyalkyl, alkenyloxyalkyl, alkynyloxyalkyl, alkoxyalkenyl, alkoxyalkynyl, cycloalkyloxy, cycloalkylalkoxy, alkoxyalkoxy, alkenyloxyalkoxy, alkynyloxyalkoxy, carboxyl, alkoxycarbonyl, alkenyloxycarbonyl, alkynyloxycarbonyl, alkylcarbonyl, alkenylcarbonyl, alkynylcarbonyl, arylalkoxy, amino, mono or di(alkyl)amino, aminoalkyl, aminoalkenyl, aminoalkynyl, mono or di(alkyl)aminoalkyl, mono or di(alkyl)aminoalkenyl, mono or di(alkyl)aminoalkynyl, mono or di(alkyl)aminocarbonyl, heterocyclyl, heteroaryl, heterocyclylalkyl, heteroarylalkyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, arylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{4a};
and/or two Z⁴ together with the atom(s) to which they are attached can form an aryl, a cycloalkyl, a heteroaryl, or a heterocyclyl, wherein each of said aryl, heteroaryl, cycloalkyl, and heterocyclyl can be unsubstituted or substituted with one or more Z^{4a};
each Z^{4a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
with the proviso that
when R¹ is A¹-X¹—, X¹ is —CO—, and A¹ is heterocyclyl, then A¹ is not attached to X¹ via an N ring atom of said heterocyclyl;
when R¹ is a heteroaryl, R¹ is not oxadiazolyl;
when R² is A²-X²—, X² is —CO—, and A² is heterocyclyl, then A² is not attached to X² via an N ring atom of said heterocyclyl; and
when R² is a heteroaryl, R² is not oxadiazolyl;
with the proviso that said compound is not
N,4-bis(4-methylphenyl)-1H-pyrrole-3-sulfonamide; (CAS no 1427286-05-2),
N,4-bis(4-chlorophenyl)-1H-pyrrole-3-sulfonamide (CAS no 1427286-06-3).

The present invention also provides, in a second aspect, a pharmaceutical composition comprising a pharmaceutically acceptable carrier, and as active ingredient an effective amount of a compound according to the first aspect of the invention or a pharmaceutically acceptable salt thereof.

The present invention also encompasses the compound according to the invention or a pharmaceutical composition according to the invention for use as a medicine. The present invention also encompasses the compound according to the invention or a pharmaceutical composition according to the invention for use in the prevention and/or treatment of GPR17 mediated disorders in a subject or a patient in need thereof, preferably in an animal, for example a mammal such a human in need thereof.

The present invention also relates to a method of treatment and/or prevention of GPR17 mediated disorders in a subject or patient in need thereof by the administration of one or more of said compounds, optionally in combination with one or more other medicines, to the subject or patient in need thereof.

The above and other characteristics, features, and advantages of the present invention will become apparent from the following detailed description, which illustrate, by way of example, the principles of the invention.

### DETAILED DESCRIPTION OF THE INVENTION

When describing the invention, the terms used are to be construed in accordance with the following definitions, unless a context dictates otherwise.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. When describing the compounds, processes, method and uses of the invention, the terms used are to be construed in accordance with the following definitions, unless the context dictates otherwise.

As used herein, the singular forms “a”, “an”, and “the” include both singular and plural referents unless the context clearly dictates otherwise. By way of example, “a compound” means one compound or more than one compound.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The terms “comprising”, “comprises” and “comprised of” as used herein are synonymous with “including”, “includes” or “containing”, “contains”, and are inclusive or open-ended and do not exclude additional, non-recited members, elements, or method steps. The terms “comprising”, “comprises” and “comprised of” also include the term “consisting of”.

The recitation of numerical ranges by endpoints includes all integer numbers and, where appropriate, fractions subsumed within that range (e.g., 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g., from 1.0 to 5.0 includes both 1.0 and 5.0). Any numerical range recited herein is intended to include all sub-ranges subsumed therein.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims and statements, any of the embodiments can be used in any combination.

The term “leaving group” or “LG” as used herein means a chemical group which is susceptible to be displaced by a nucleophile or cleaved off or hydrolyzed in basic or acidic conditions. In a particular embodiment, a leaving group is selected from a halogen atom (e.g., Cl, Br, I) or a sulfonate (e.g., mesylate, tosylate, triflate).

The term “protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e., a compound that is sufficiently robust to survive isolation from a reaction mixture.

The term “halo” or “halogen” as a group or part of a group is generic for fluoro, chloro, bromo, iodo.

The term “cyano” as used herein refers to the group —CN.

The term “oxo” as used herein refers to the group ═O.

The term “nitro” as used herein refers to the group —NO₂.

The term “thioxo” as used herein refers to the group ═S.

The term “hydroxyl” or “hydroxy” as used herein refers to the group —OH.

The term “thio” or “thiol” as used herein refers to the group —SH The term “alkyl” as a group or part of a group, refers to a hydrocarbyl group of formula CₙH₂ₙ₊₁ wherein n is a number greater than or equal to 1, with no site of unsaturation. Alkyl groups may be linear or branched and may be substituted as indicated herein. Generally, alkyl groups of this invention comprise from 1 to 18 carbon atoms, preferably from 1 to 10 carbon atoms, more preferably from 1 to 6 carbon atoms, more preferably from 1 to 4 carbon atoms. When a subscript is used herein following a carbon atom, the subscript refers to the number of carbon atoms that the named group may contain. For example, the term “C₁₋₆alkyl”, as a group or part of a group, refers to a hydrocarbyl group of formula CₙH₂ₙ₊₁ wherein n is a number ranging from 1 to 6. Thus, for example, “C₁₋₆alkyl” includes all linear or branched alkyl groups with between 1 and 6 carbon atoms, and thus includes methyl, ethyl, n-propyl, i-propyl, butyl, and its isomers (e.g., n-butyl, i-butyl, and t-butyl); pentyl and its isomers, hexyl, and its isomers, etc. For example, C₁₋₄alkyl includes all linear or branched alkyl groups having 1 to 4 carbon atoms, and thus includes for example methyl, ethyl, n-propyl, i-propyl, 2-methyl-ethyl, butyl, and its isomers (e.g., n-butyl, i-butyl, and t-butyl), and the like. In particular embodiments, the term alkyl refers to C₁₋₁₂alkyl (C₁₋₁₂ hydrocarbons), yet more in particular to C₁₋₉alkyl (C₁₋₉ hydrocarbons), yet more in particular to C₁₋₆alkyl (C₁₋₆ hydrocarbons) as further defined herein above. Non-limiting examples of alkyl include methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, and n-icosyl.

When the suffix “ene” is used in conjunction with an alkyl group, i.e., “alkylene”, this is intended to mean the alkyl group as defined herein having two single bonds as points of attachment to other groups. As used herein, the term “alkylene” also referred as “alkanediyl”, by itself or as part of another substituent, refers to alkyl groups that are divalent, i.e., having two monovalent group centers derived by the removal of two hydrogen atoms from the same or two different carbon atoms of a parent alkane, i.e., with two single bonds for attachment to two other groups. Alkylene groups may be linear or branched and may be substituted as indicated herein. Non-limiting examples of alkylene groups include methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and its chain isomers, hexylene and its chain isomers.

The term “hydrocarbyl” group is used herein in accordance with the definition specified by IUPAC as follows: a univalent group formed by removing a hydrogen atom from a hydrocarbon (that is, a group containing only carbon and hydrogen).

The term “alkenyl” as a group or part of a group, refers to an unsaturated hydrocarbyl group which may be linear, or branched, comprising one or more with at least one site (usually 1 to 3, preferably 1) of unsaturation, namely at least one sp² carbon-sp² carbon double bond. Generally, alkenyl groups of this invention comprise from 2 to 20 carbon atoms, preferably from 2 to 10 carbon atoms, preferably from 2 to 8 carbon atoms, more preferably 2 to 6 carbon atoms. When a subscript is used herein following a carbon atom, the subscript refers to the number of carbon atoms that the named group may contain. Examples of C₂₋₆alkenyl groups are ethenyl, 2-propenyl, 2-butenyl, 3-butenyl, 2-pentenyl and its isomers, 2-hexenyl and its isomers, 2,4-pentadienyl, and the like. The double bond may be in the cis or trans configuration.

When the suffix “ene” is used in conjunction with an alkenyl group, i.e., “alkenylene”, this is intended to mean the alkenyl group as defined herein having two single bonds as points of attachment to other groups. As used herein, the term “alkenylene” by itself or as part of another substituent, refers to alkenyl groups that are divalent, i.e., having two monovalent centers derived by the removal of two hydrogen atoms from the same or two different carbon atoms of a parent alkene, i.e., with two single bonds for attachment to two other groups. Alkenylene groups may be linear or branched and may be substituted as indicated herein. Non-limiting examples of alkenylene groups include —CH═CH—, —C(CH₃)═CH—, —C(CH₃)═C(CH₃)—, —CH═CH—CH₂—, —CH₂—C(CH₃)═CH—, —CH₂—CH═C(CH₃)—, —CH₂—CH₂—CH═CH—, and the like.

The term “alkynyl” as a group or part of a group, refers to a branched or straight chain hydrocarbon comprising at least one site (usually 1 to 3, preferably 1) of unsaturation, namely a sp¹ carbon-sp¹ carbon triple bond. In particular embodiments, the term alkynyl refers to C₂₋₁₂-alkynyl (C₂₋₁₂ hydrocarbons), preferably to C₂₋₉ alkynyl (C₂₋₉ hydrocarbons) yet more preferably to C₂₋₆ alkynyl (C₂₋₆ hydrocarbons) as further defined herein above with at least one site (usually 1 to 3, preferably 1) of unsaturation, namely at least one sp¹ carbon-sp¹ carbon triple bond. Examples of alkynyl include but are not limited to: ethynyl (—C≡CH), 3-ethyl-cyclohept-1-ynylene, and 1-propynyl (propargyl, —CH₂C≡CH).

When the suffix “ene” is used in conjunction with an alkynyl group, i.e., “alkynylene”, this is intended to mean the alkynyl group as defined herein having two single bonds as points of attachment to other groups. As used herein, the term “alkynylene” by itself or as part of another substituent, refers to alkynyl groups that are divalent, i.e., with two single bonds for attachment to two other groups. Alkynylene groups may be linear or branched and may be substituted as indicated herein. Non-limiting examples of alkynylene groups include —C≡C—, —CH₂—C≡C—, —C≡C—CH₂—, —CH₂—CH₂—C≡C—, and the like.

The term “cycloalkyl”, as a group or part of a group, refers to a cyclic alkyl group, that is a monovalent, saturated, hydrocarbyl group having 1 or more cyclic structure, and comprising from 3 to 20 carbon atoms, more preferably from 3 to 10 carbon atoms, more preferably from 3 to 8 carbon atoms; more preferably from 3 to 6 carbon atoms. Cycloalkyl includes all saturated hydrocarbon groups containing 1 or more rings, including monocyclic, bicyclic groups or tricyclic. For example, cycloalkyl comprises a C₃₋₁₀ monocyclic or C₇₋₁₈ polycyclic saturated hydrocarbon, such as for instance cyclopropyl, cyclobutyl, cyclopentyl, cyclopropylethylene, methylcyclopropylene, cyclohexyl, cycloheptyl, cyclooctyl, cyclooctyl methylene, norbornyl, fenchyl, trimethyltricycloheptyl, decalinyl, adamantyl and the like. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₁₀cycloalkyl”, refers to a cyclic alkyl group comprising from 3 to 10 carbon atoms. For example, the term “C₃₋₈cycloalkyl”, refers to a cyclic alkyl group comprising from 3 to 8 carbon atoms. For example, the term “C₃₋₆cycloalkyl”, refers to a cyclic alkyl group comprising from 3 to 6 carbon atoms. For the avoidance of doubt, fused systems of a cycloalkyl ring with a heterocyclic ring are considered as heterocycle irrespective of the ring that is bound to the core structure. Fused systems of a cycloalkyl ring with an aryl ring are considered as aryl irrespective of the ring that is bound to the core structure. Fused systems of a cycloalkyl ring with a heteroaryl ring are considered as heteroaryl irrespective of the ring that is bound to the core structure.

The term “cycloalkenyl” as a group or part of a group, refers to a non-aromatic cyclic alkenyl group, with at least one site (usually 1 to 3, preferably 1) of unsaturation, namely a sp² carbon-sp² carbon double bond; preferably from 4 to 18 carbon atoms, more preferably from 4 to 10 carbon atoms, more preferably from 5 to 6 carbon atoms. Cycloalkenyl includes all unsaturated hydrocarbon groups containing 1 or more rings, including monocyclic, bicyclic, or tricyclic groups. For example, cycloalkenyl can comprise C₄₋₁₀ monocyclic or C₇₋₁₈ polycyclic hydrocarbon. The further rings may be either fused, bridged and/or joined through one or more Spiro atoms. When a subscript is used herein following a carbon atom, the subscript refers to the number of carbon atoms that the named group may contain. For example, the term “C₅₋₁₀cycloalkenyl”, refers to a cyclic alkenyl group comprising from 5 to 10 carbon atoms. For example, the term “C₅₋₈cycloalkenyl”, refers to a cyclic alkenyl group comprising from 5 to 8 carbon atoms. For example, the term “C₅₋₆cycloalkyl”, refers to a cyclic alkenyl group comprising from 5 to 6 carbon atoms. Examples include but are not limited to: cyclobutenyl, cyclopentenyl (—C₅H₇), cyclopentenylpropylene, methylcyclohexenylene, and cyclohexenyl (—C₆H₉). The double bond may be in the cis or trans configuration. For the avoidance of doubt, fused systems of a cycloalkenyl ring with a heterocyclic ring are considered as heterocycle irrespective of the ring that is bound to the core structure. Fused systems of a cycloalkenyl ring with an aryl ring are considered as aryl irrespective of the ring that is bound to the core structure. Fused systems of a cycloalkenyl ring with a heteroaryl ring are considered as heteroaryl irrespective of the ring that is bound to the core structure.

The term “cycloalkynyl” as a group or part of a group, to a non-aromatic hydrocarbon group preferably having from 5 to 18 carbon atoms with at least one site (usually 1 to 3, preferably 1) of unsaturation, namely a sp¹ carbon-sp¹ carbon triple bond and consisting of or comprising a C₅₋₁₀ monocyclic or C₇₋₁₈ polycyclic hydrocarbon. Examples include but are not limited to: cyclohept-1-yne, 3-ethyl-cyclohept-1-ynylene, 4-cyclohept-1-yn-methylene and ethylene-cyclohept-1-yne. In particular embodiments, the term cycloalkynyl refers to C₅₋₁₀ cycloalkynyl (cyclic C₅₋₁₀ hydrocarbons), preferably to C₅₋₉ cycloalkynyl (cyclic C₅₋₉ hydrocarbons), yet more preferably to C₅₋₆ cycloalkynyl (cyclic C₅₋₉ hydrocarbons) as further defined herein above with at least one site (usually 1 to 3, preferably 1) of unsaturation, namely a sp¹ carbon-sp¹ carbon triple bond. For the avoidance of doubt, fused systems of a cycloalkynyl ring with a heterocyclic ring are considered as heterocycle irrespective of the ring that is bound to the core structure. Fused systems of a cycloalkynyl ring with an aryl ring are considered as aryl irrespective of the ring that is bound to the core structure. Fused systems of a cycloalkynyl ring with a heteroaryl ring are considered as heteroaryl irrespective of the ring that is bound to the core structure.

The term “cycloalkylalkyl” or “cycloalkyl-alkyl”, as a group or part of a group, refers to a group of formula —R^{a}—R^{g} wherein R^{g} is cycloalkyl, and R^{a} is alkylene as defined herein.

The term “cycloalkenylalkyl” or “cycloalkenyl-alkyl”, as a group or part of a group, refers to a group of formula —R^{a}—R^{t} wherein R^{t} is cycloalkenyl, and R^{a} is alkylene as defined herein.

The term “cycloalkynylalkyl” or “cycloalkynyl-alkyl”, as a group or part of a group, refers to a group of formula —R^{a}—R^{s} wherein R^{s} is cycloalkynyl, and R^{a} is alkylene as defined herein.

The term “alkoxy” or “alkyloxy”, as a group or part of a group, refers to a group of formula —OR^{b} wherein Rb is alkyl as defined herein. Non-limiting examples of suitable C₁₋₆alkoxy include methoxy, ethoxy, propoxy, isopropoxy, butoxy, isobutoxy, sec-butoxy, tert-butoxy, pentyloxy, and hexyloxy.

The term “alkenyloxy”, as a group or part of a group, refers to a group of formula —OR^{d} wherein Rd is alkenyl as defined herein.

The term “alkynyloxy”, as a group or part of a group, refers to a group of formula —OR^{e} wherein R^{e} is alkynyl as defined herein.

The term “alkoxyalkyl” or “alkyloxyalkyl”, as a group or part of a group, refers to a group of formula —R^{a}—OR^{b} wherein R^{a} is alkylene and Rb is alkyl as defined herein.

The term “alkenyloxyalkyl”, as a group or part of a group, refers to a group of formula —R^{a}—OR^{d} wherein R^{a} is alkylene and Rd is alkenyl as defined herein.

The term “alkynyloxyalkyl”, as a group or part of a group, refers to a group of formula —R^{a}—OR^{d} wherein R^{a} is alkylene and R^{c} is alkynyl as defined herein.

The term “alkoxyalkenyl” or “alkyloxyalkenyl”, as a group or part of a group, refers to a group of formula —R^{h}—OR^{b}, wherein R^{h} is alkenylene and R^{b} is alkyl as defined herein.

The term “alkoxyalkynyl” or “alkynyloxyalkynyl”, as a group or part of a group, refers to a group of formula —Rⁱ—OR^{b} wherein Rⁱ is alkynylene and R^{b} is alkyl as defined herein.

The term “cyanoalkyl”, as a group or part of a group, refers to a group of formula —R^{a}—CN wherein R^{a} is alkylene as defined herein.

The term “cyanoalkoxy” or “cyanoalkyloxy”, as a group or part of a group, refers to a group of formula —O—R^{a}—CN wherein R^{a} is alkylene as defined herein.

The term “cycloalkoxy”, as a group or part of a group, refers to a group of formula —OR^{g} wherein R^{g} is cycloalkyl as defined herein.

The term “cycloalkylalkoxy”, as a group or part of a group, refers to a group of formula —O—R^{a}—R^{g} wherein R^{a} is alkylene and R^{g} is cycloalkyl as defined herein.

The term “alkoxyalkoxy” or “alkyloxyalkyloxy”, as a group or part of a group, refers to a group of formula —O—R^{a}—OR^{b} wherein R^{a} is alkylene and R^{b} is alkyl as defined herein.

The term “alkenyoxyalkoxy” or “alkenyloxyalkyloxy”, as a group or part of a group, refers to a group of formula —O—R^{a}—OR^{d} wherein R^{a} is alkylene and Rd is alkenyl as defined herein.

The term “alkynyoxyalkoxy” or “alkynyloxyalkyloxy”, as a group or part of a group, refers to a group of formula —O—R^{a}—OR^{c} wherein R^{a} is alkylene and R^{c} is alkynyl as defined herein.

The term “aryl”, as a group or part of a group, refers to a polyunsaturated, aromatic hydrocarbyl group having a single ring (i.e., phenyl) or multiple aromatic rings fused together (e.g., naphthyl), or linked covalently, typically containing 6 to 20 atoms; preferably 6 to 10, wherein at least one ring is aromatic. Typical aryl groups include, but are not limited to 1 ring, or 2 or 3 rings fused together, derived from benzene, naphthalene, anthracene, biphenyl, and the like. The aromatic ring may optionally include one to two additional rings. Fused systems of an aryl ring with a cycloalkyl ring, or a cycloalkenyl ring, or a cycloalkynyl ring, are considered as aryl irrespective of the ring that is bound to the core structure. Fused systems of an aryl ring with a heterocycle are considered as heterocycle irrespective of the ring that is bound to the core structure. Fused systems of an aryl ring with a heteroaryl are considered as heteroaryl irrespective of the ring that is bound to the core structure. Examples of suitable aryl include C₆₋₂₀aryl, preferably C₆₋₁₀aryl, more preferably C₆₋₉aryl. Non-limiting examples of aryl comprise phenyl, biphenylyl, biphenylenyl, or 1- or 2-naphthanelyl; 1-, 2-, 3-, 4-, 5- or 6-tetralinyl (also known as “1,2,3,4-tetrahydronaphtalene); 1-, 2-, 3-, 4-, 5-, 6-, 7- or 8-azulenyl, 4-, 5-, 6 or 7-indenyl; 4- or 5-indanyl; 5-, 6-, 7- or 8-tetrahydronaphthyl; 1,2,3,4-tetrahydronaphthyl; and 1,4-dihydronaphthyl; 1-, 2-, 3-, 4- or 5-pyrenyl.

The term “arylalkyl”, as a group or part of a group, refers to an alkyl as defined herein, wherein at least one hydrogen atom is replaced by at least one aryl as defined herein. Non-limiting examples of arylalkyl group include benzyl, phenethyl, dibenzylmethyl, benzyl, 2-phenylethan-1-yl, 2-phenylethen-1-yl, naphthylmethyl, 2-naphthylethyl, and the like. The term “C₆₋₁₀arylC₁₋₆alkyl” means that the alkyl moiety of the arylalkyl group can comprises 1 to 6 carbon atoms and the aryl moiety is 6 to 10 carbon atoms.

The term “arylalkenyl” as a group or part of a group, refers to an alkenyl in which one of the hydrogen atoms bonded to a carbon atom, is replaced with an aryl. The term “C₆₋₁₀arylC₂₋₆alkenyl” means that the alkenyl moiety of the arylalkenyl group can comprise 2 to 6 carbon atoms and the aryl moiety 6 to 10 carbon atoms.

The term “arylalkynyl” as a group or part of a group, refers to an alkynyl in which one of the hydrogen atoms bonded to a carbon atom, is replaced with an aryl. The term “C₆₋₁₀arylC₂₋₆alkynyl” means that the alkenyl moiety of the arylalkynyl group can comprise 2 to 6 carbon atoms and the aryl moiety 6 to 10 carbon atoms.

The term “aryloxy”, as a group or part of a group, refers to a group of formula —O—R^{f} wherein R^{f} is aryl as defined herein.

The term “arylalkoxy” or “arylalkyloxy”, as a group or part of a group, refers to a group of formula —O—R²—R^{f} wherein R^{f} is aryl, and R^{a} is alkylene as defined herein.

The term “aryloxyalkyl”, as a group or part of a group, refers to a group of formula —R^{a}—O—R^{f}wherein R^{f} is aryl, and R^{a} is alkylene as defined herein.

The term “aryloxyalkenyl”, as a group or part of a group, refers to a group of formula —R^{h}—O—R^{f} wherein R^{f} is aryl, and R^{h} is alkenylene as defined herein.

The term “aryloxyalkynyl”, as a group or part of a group, refers to a group of formula —R¹—O—R^{f} wherein R^{f} is aryl, and Rⁱ is alkynylene as defined herein.

The term “arylthio”, as a group or part of a group, refers to a group of formula —S—R^{f} wherein R^{f} is aryl as defined herein.

The term “haloalkyl”, as a group or part of a group, refers to an alkyl group having the meaning as defined herein, wherein one or more hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl and the like.

The term “haloalkenyl”, as a group or part of a group, refers to an alkenyl group having the meaning as defined herein, wherein one or more hydrogen atoms are each replaced with a halogen as defined herein.

The term “haloalkynyl”, as a group or part of a group, refers to an alkynyl group having the meaning as defined herein, wherein one or more hydrogen atoms are each replaced with a halogen as defined herein.

The term “alkylthio”, as a group or part of a group, refers to a group of formula —S—R^{b} wherein R^{b} is alkyl as defined herein. Non-limiting examples of alkylthio groups include methylthio (—SCH₃), ethylthio (—SCH₂CH₃), n-propylthio, isopropylthio, n-butylthio, isobutylthio, sec-butylthio, tert-butylthio and the like.

The term “alkenylthio”, as a group or part of a group, refers to a group of formula —S—R^{d} wherein R^{d} is alkenyl as defined herein.

The term “alkynylthio”, as a group or part of a group, refers to a group of formula —S—R^{c} wherein R^{c} is alkynyl as defined herein.

The term “haloalkylthio”, as a group or part of a group, refers to a group of formula —S—R^{e}, wherein Re is haloalkyl as defined herein.

The term “cycloalkylthio”, as a group or part of a group, refers to a group of formula —S—R^{g}, wherein R^{g} is cycloalkyl as defined herein.

The term “haloalkoxy”, as a group or part of a group, refers to a group of formula —O—R^{e}, wherein Re is haloalkyl as defined herein. Non-limiting examples of suitable haloalkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy, trichloromethoxy, 2-bromoethoxy, pentafluoroethyl, 3,3,3-trichloropropoxy, 4,4,4-trichlorobutoxy.

The term “haloalkenyloxy”, as a group or part of a group, refers to a group of formula —O—R^{j}, wherein R^{j} is haloalkenyl as defined herein.

The term “haloalkynyloxy”, as a group or part of a group, refers to a group of formula —O—R^{k}, wherein R^{k} is haloalkynyl as defined herein.

The term “hydroxyalkyl”, as a group or part of a group, refers to a group of formula —R^{a}—OH wherein R^{a} is alkylene as defined herein.

The term “hydroxyalkenyl”, as a group or part of a group, refers to a group of formula —R^{h}—OH wherein R^{h} is alkenylene as defined herein.

The term “hydroxyalkynyl”, as a group or part of a group, refers to a group of formula —Rⁱ—OH wherein Rⁱ is alkynylene as defined herein.

The term “carboxy”, “carboxyl” or “hydroxycarbonyl”, as a group or part of a group, refers to the group —C(═O)—OH.

The term “carbonyl” as a group or part of a group, refers to the group —C(═O)—, also written as —CO—.

The term “alkoxycarbonyl” or “alkyloxycarbonyl”, as a group or part of a group, refers to a group of formula —C(═O)—O—R^{b}, wherein R^{b} is alkyl as defined herein.

The term “alkenyloxycarbonyl”, as a group or part of a group, refers to a group of formula —C(═O)—O—R^{d}, wherein R^{d} is alkenyl as defined herein.

The term “alkynyloxycarbonyl”, as a group or part of a group, refers to a group of formula —C(═O)—O—R^{c}, wherein R^{C} is alkynyl as defined herein.

The term “alkylcarbonyl”, as a group or part of a group, refers to a group of formula —C(═O)—R^{b}, wherein R^{b} is alkyl as defined herein.

The term “alkenylcarbonyl”, as a group or part of a group, refers to a group of formula —C(═O)—R^{d}, wherein R^{d} is alkenyl as defined herein.

The term “alkynylcarbonyl”, as a group or part of a group, refers to a group of formula —C(═O)—R^{c}, wherein R^{c} is alkynyl as defined herein.

The term “cycloalkylcarbonyl”, as a group or part of a group, refers to a group of formula —C(═O)—R^{g}, wherein R^{g} is cycloalkyl as defined herein.

The term “arylcarbonyl”, as a group or part of a group, refers to a group of formula —C(═O)—R^{f}, wherein R^{f} is aryl as defined herein.

The term “amino” as a group or part of a group, refers to the —NH₂ group.

The term “mono- or di-alkylamino”, as a group or part of a group, refers to a group of formula —N(R^{l})(R^{b}), wherein R^{l} is hydrogen or alkyl, R^{b} is alkyl as defined herein. Thus, such term includes mono-alkyl amino group (e.g., mono-alkylamino group such as methylamino and ethylamino), and di-alkylamino group (e.g., di-alkylamino group such as dimethylamino and diethylamino). Non-limiting examples of suitable mono- or di-alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-1-propylamino, n-butylmethylamino, i-butylmethylamino, t-butylmethylamino, ethyl-n-propylamino, ethyl-1-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “aminoalkyl”, as a group or part of a group, refers to a group of formula —R^{a}—NH₂ wherein R^{a} is alkylene as defined herein.

The term “aminoalkenyl”, as a group or part of a group, refers to a group of formula —R^{h}—NH₂ wherein R^{h} is alkenylene as defined herein.

The term “aminoalkynyl”, as a group or part of a group, refers to a group of formula —Rⁱ—NH₂ wherein Rⁱ is alkynylene as defined herein.

The term “mono or di(alkyl)aminoalkyl”, as a group or part of a group, refers to a group of formula —R^{a}—N(R^{l})(R^{b}), wherein R^{a} is alkylene, R^{l} is hydrogen or alkyl, R^{b} is alkyl as defined herein.

The term “mono or di(alkyl)aminoalkenyl”, as a group or part of a group, refers to a group of formula —Rⁱ—N(R^{l})(R^{b}), wherein R^{h} is alkenylene, R^{l} is hydrogen or alkyl, R^{b} is alkyl as defined herein.

The term “mono or di(alkyl)aminoalkynyl”, as a group or part of a group, refers to a group of formula —R—N(R^{l})(R^{b}), wherein R^{l} is alkynylene, R^{b} is hydrogen or alkyl, R^{b} is alkyl as defined herein.

The term “mono or di(alkyl)aminocarbonyl”, as a group or part of a group, refers to a group of formula —C(═O)—N(R^{l})(R^{b}), wherein R^{l} is hydrogen or alkyl, R^{b} is alkyl as defined herein.

The term “heterocycle” or “heterocyclyl” as used herein refer to non-aromatic, fully saturated or partially unsaturated ring system comprising from 3 to 18 atoms including at least one N, O, S, or P, preferably 3 to 14 atoms (3-14 membered heterocyclyl) (for example, 3 to 7 member monocyclic, 7 to 14 member bicyclic, preferably comprising a total of 3 to 10 ring atoms (3-10 membered heterocyclyl), more preferably 4 to 10 atoms (4-10 membered heterocyclyl), yet more preferably 5 to 10 atoms (5-10 membered heterocyclyl). Each ring of the heterocycle or heterocyclyl may have 1, 2, 3 or 4 heteroatoms selected from N, O, P and/or S, where the N and S heteroatoms may optionally be oxidized, and the N heteroatoms may optionally be quaternized, and wherein at least one carbon atom of heterocyclyl can be oxidized to form at least one C═O. The heterocyclyl may be attached at any heteroatom or carbon atom of the ring or ring system, where valence allows. The rings of multi-ring heterocyclyls or heterocycles may be fused, bridged and/or joined through one or more spiro atoms. Fused systems of a heterocycle or heterocyclyl with an aryl ring are considered as heterocycle or heterocyclyl irrespective of the ring that is bound to the core structure. Fused systems of a heterocycle or heterocyclyl with a heteroaryl ring are considered as heteroaryl irrespective of the ring that is bound to the core structure.

Non limiting exemplary heterocycles or heterocyclic groups include piperidinyl, piperazinyl, homopiperazinyl, morpholinyl, tetrahydropyranyl, tetrahydrofuranyl, pyrrolidinyl, aziridinyl, oxiranyl, thiiranyl, azetidinyl, oxetanyl, thietanyl, imidazolinyl, pyrazolidinyl imidazolidinyl, oxazolinyl, isoxazolinyl, oxazolidinyl, isoxazolidinyl, thiazolidinyl, isothiazolidinyl, succinimidyl, indolinyl, isoindolinyl, chromanyl (also known as 3,4-dihydrobenzo[b]pyranyl), 2H-pyrrolyl, pyrrolinyl (such as 1-pyrrolinyl, 2-pyrrolinyl, 3-pyrrolinyl), 4H-quinolizinyl, 2-oxopiperazinyl, pyrazolinyl (such as 2-pyrazolinyl, 3-pyrazolinyl), tetrahydro-2H-pyranyl, 2H-pyranyl, 4H-pyranyl, dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formyl-piperazinyl, thiomorpholinyl, dihydrofuranyl, dihydrothienyl, tetrahydrothienyl, dihydropyrazolyl, dihydroimidazolyl, isothiazolinyl, thiazolinyl, triazolinyl, triazolidinyl, oxadiazolinyl, oxadiazolidinyl, thiadiazolinyl, thiadiazolidinyl, tetrazolinyl, tetrazolidinyl, dihydro-pyridinyl, tetrahydro-pyridinyl, 1,2,3,6-tetrahydropyridinyl, hexahydro-pyridinyl, dihydro-pyrimidinyl, tetrahydro-pyrimidinyl, 1,4,5,6-tetrahydropyrimidinyl, dihydro-pyrazinyl, tetrahydro-pyrazinyl, dihydro-pyridazinyl, tetrahydro-pyridazinyl, dihydro-triazinyl, tetrahydro-triazinyl, hexahydro-triazinyl, 1,4-diazepanyl, dihydro-indolyl, indolinyl, tetrahydro-indolyl, dihydro-indazolyl, tetrahydro-indazolyl, dihydro-isoindolyl, dihydro-benzofuranyl, tetrahydro-benzofuranyl, dihydro-benzothienyl, tetrahydro-benzothienyl, dihydro-benzimidazolyl, tetrahydro-benzimidazolyl, dihydro-benzooxazolyl, 2,3-dihydrobenzo[d]oxazolyl, tetrahydro-benzooxazolyl, dihydro-benzooxazinyl, 3,4-dihydro-2H-benzo[b][1,4]oxazinyl, tetrahydro-benzooxazinyl, benzo[1,3]dioxolyl, benzo[1,4]dioxanyl, dihydro-purinyl, tetrahydro-purinyl, dihydro-quinolinyl, 1,2,3,4-tetrahydroquinolinyl, dihydro-isoquinolinyl, 3,4-dihydroisoquinolin-(1H)-yl, tetrahydro-isoquinolinyl, 1,2,3,4-tetrahydroisoquinolinyl, dihydro-quinazolinyl, tetrahydro-quinazolinyl, dihydro-quinoxalinyl, tetrahydro-quinoxalinyl, 1,2,3,4-tetrahydroquinoxalinyl, 2,5-dihydro-1H-pyrrolyl, 4,5-dihydro-1H-imidazolyl, hexahydropyrrolo[3,4-b][1,4]oxazin-(2H)-yl, 3,4-dihydro-2H-pyrido[3,2-b][1,4]oxazinyl, (cis)-octahydrocyclopenta[c]pyrrolyl, hexahydropyrrolo[3,4-b]pyrrol-(1H)-yl, 5H-pyrrolo[3,4-b]pyridin-(7H)-yl, 5,7-dihydro-6H-pyrrolo[3,4-b]pyridinyl, tetrahydro-1H-pyrrolo[3,4-b]pyridin-(2H,7H,7aH)-yl, hexahydro-1H-pyrrolo[3,4-b]pyridin-(2H)-yl, (octahydro-6H-pyrrolo[3,4-b]pyridinyl, hexahydropyrrolo[1,2-a]pyrazin-(1H)-yl, 3,4,6,7,8,8a-hexahydro-1H-pyrrolo[1,2-a]pyrazinyl, 2,3,4,9-tetrahydro-1H-carbazolyl, 1,2,3,4-tetrahydropyrazino[1,2-a]indolyl, 2,3-dihydro-1H-pyrrolo[1,2-a]indolyl, 1,3-dihydro-2H-isoindolyl, octahydro-2H-isoindolyl, 2,5-diazabicyclo[2.2.1]heptanyl, 2-azabicyclo[2.2.1]heptenyl, 3-azabicyclo[3.1.0]hexanyl, 3,6-diazabicyclo[3.1.0]hexanyl, 5-azaspiro[2.4]heptanyl, 4,7-diazaspiro[2.5]octanyl, 2,6-diazaspiro[3.3]heptanyl, 2,5-diazaspiro[3.4]octanyl, 2,6-diazaspiro[3.4]octanyl, 2,7-diazaspiro[3.5]nonanyl, 2,7-diazaspiro[4.4]nonanyl, 2-azaspiro[4.5]decanyl, 2,8-diazaspiro[4.5]decanyl, 3,6-diazabicyclo[3.2.1]octyl, 1,4-dihydroindeno[1,2-c]pyrazolyl, dihydropyranyl, dihydropyridinyl, dihydroquinolinyl, 8H-indeno[1,2-d]thiazolyl, tetrahydroimidazo[1,2-a]pyridinyl, pyridin-2(1H)-one, 8-azabicyclo[3.2.1]oct-2-enyl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxan-5-yl, 1,2,4-trioxan-3-yl, 1,2,4-trioxan-5-yl, 1,2,4-trioxan-6-yl and 1,3,4-trioxan-2-yl. The term “azepanyl” as used herein includes azepan-1-yl, azepan-2-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “chromanyl” as used herein includes chroman-2-yl, chroman-3-yl, chroman-4-yl, chroman-5-yl, chroman-6-yl, chroman-7-yl and chroman-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heterocyclylalkyl” or “heterocyclyl-alkyl”, as a group or part of a group, refers to an alkyl as defined herein, wherein at least one hydrogen atom is replaced by at least one heterocyclyl as defined herein, and can be represented by a group of formula —R^{a}—R^{o} wherein R^{a} is alkylene and R^{o} is heterocyclyl as defined herein. The term “3 to 10 membered heterocyclyl-C₁₋₆alkyl” refers to a heterocyclyl-alkyl wherein the alkylene moiety comprises from 1 to 6 carbon atoms and the heterocyclyl moiety is non-aromatic, fully saturated or partially unsaturated ring system of 3 to 10 atoms including at least one N, O, S, or P.

The term “heterocyclylalkenyl” or “heterocyclyl-alkenyl”, as a group or part of a group, refers to an alkenyl as defined herein, wherein at least one hydrogen atom is replaced by at least one heterocyclyl as defined herein, and can be represented by a group of formula —R^{h}—R^{o} wherein R^{h} is alkenylene and R^{o} is heterocyclyl as defined herein. The term “3 to 10 membered heterocyclyl-C₂₋₆alkenyl” refers to a heterocyclyl-alkenyl wherein the alkenylene moiety comprises from 2 to 6 carbon atoms and the heterocyclyl moiety is non-aromatic, fully saturated or partially unsaturated ring system of 3 to 10 atoms including at least one N, O, S, or P.

The term “heterocyclylalkynyl” or “heterocyclyl-alkynyl”, as a group or part of a group, refers to an alkynyl as defined herein, wherein at least one hydrogen atom is replaced by at least one heterocyclyl as defined herein, and can be represented by a group of formula —Rⁱ—R^{o} wherein Rⁱ is alkynylene and R^{o} is heterocyclyl as defined herein. The term “3 to 10 membered heterocyclyl-C₂₋₆alkynyl” refers to a heterocyclyl-alkynyl wherein the alkynylene moiety comprises from 2 to 6 carbon atoms and the heterocyclyl moiety is non-aromatic, fully saturated or partially unsaturated ring system of 3 to 10 atoms including at least one N, O, S, or P.

The term “heteroaryl” refers to an aromatic ring system comprising from 5 to 18 atoms including at least one N, O, S, or P, containing 1 or 2 rings which can be fused together or linked covalently, preferably 5 to 14 atoms (5-14 membered heteroaryl), yet more preferably 5 to 10 atoms (5-10 membered heteroaryl), each ring typically containing 5 to 6 atoms; at least one of said rings is aromatic, where the N and S heteroatoms may optionally be oxidized and the N heteroatoms may optionally be quaternized, and wherein at least one carbon atom of said heteroaryl can be oxidized to form at least one C═O. Fused systems of a heteroaryl ring with a cycloalkyl ring, or a cycloalkenyl ring, or a cycloalkynyl ring, are considered as heteroaryl irrespective of the ring that is bound to the core structure. Fused systems of a heteroaryl ring with a heterocycle are considered as heteroaryl irrespective of the ring that is bound to the core structure. Fused systems of a hetero aryl ring with an aryl ring are considered as heteroaryl irrespective of the ring that is bound to the core structure. Non-limiting examples of such heteroaryl, include: pyridinyl, pyrrolyl, thiophenyl (also referred as thienyl), furanyl, thiazolyl, isothiazolyl, thiadiazolyl, triazol-2-yl, 1H-pyrazol-5-yl, pyrazolyl, imidazolyl, oxazolyl, isoxazolyl, triazolyl, oxadiazolyl, tetrazolyl, oxatriazolyl, thiatriazolyl, pyrimidinyl, pyrazinyl, pyridazinyl, oxazinyl, dioxinyl, thiazinyl, triazinyl, pyranyl, thiopyranyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, benzooxazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, benzo[c][1,2,5]oxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, 9H-purinyl, imidazo[1,2-a]pyridinyl, imidazo[1,2-a]pyrazinyl, imidazo[5,1-a]isoquinolinyl, imidazo[1,5-a]pyridinyl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; acridinyl, phthalazinyl, 1,4-dihydroindeno[1,2-c]-1H-pyrazolyl, 2,3-dihydro-1H-inden-1-one, 2,3-dihydro-1H-indenyl, 3,4-dihydroquinolin-2(1H)-one, 5,6-dihydroimidazo[5,1-a]isoquinolinyl, 8H-indeno[1,2-d]thiazolyl, benzo[d]oxazol-2(3H)-one, quinolin-2(1H)-one, quinazolin-4(1H)-one, quinazoline-2,4(1H,3H)-dione, benzo-[d]oxazolyl, and pyrazolo[1,5-a]pyridinyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl or 1H-pyrazol-5-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes triazol-2-yl, 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl, indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

Heteroaryl and heterocycle or heterocyclyl as used herein includes by way of example and not limitation these groups described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

The term “heteroarylalkyl” or “heteroaryl-alkyl”, as a group or part of a group, refers to an alkyl as defined herein, wherein at least one hydrogen atom is replaced by at least one heteroaryl as defined herein, and can be represented by a group of formula —R^{a}—R^{p} wherein R^{a} is alkylene and R^{p} is heteroaryl as defined herein. The term “5 to 10 membered heteroaryl-C₁₋₆alkyl” refers to a heteroaryl-alkyl wherein the alkylene moiety comprises from 1 to 6 carbon atoms and the heteroaryl moiety is an aromatic ring system comprising from 5 to 10 atoms including at least one N, O, S, or P.

The term “heteroarylalkenyl” or “heteroaryl-alkenyl”, as a group or part of a group, refers to an alkenyl as defined herein, wherein at least one hydrogen atom is replaced by at least one heteroaryl as defined herein, and can be represented by a group of formula —R^{h}—R^{p} wherein R^{h} is alkenylene and R^{p} is heteroaryl as defined herein. The term “5 to 10 membered heteroaryl-C₂₋₆alkenyl” refers to a heteroaryl-alkenyl wherein the alkenylene moiety comprises from 2 to 6 carbon atoms and the heteroaryl moiety is an aromatic ring system comprising from 5 to 10 atoms including at least one N, O, S, or P.

The term “heteroarylalkynyl” or “heteroaryl-alkynyl”, as a group or part of a group, refers to an alkynyl as defined herein, wherein at least one hydrogen atom is replaced by at least one heteroaryl as defined herein, and can be represented by a group of formula —Rⁱ—R^{p} wherein Rⁱ is alkynylene and R^{p} is heteroaryl as defined herein. The term “5 to 10 membered heteroaryl-C₂₋₆ alkynyl” refers to a heteroaryl-alkynyl wherein the alkynylene moiety comprises from 2 to 6 carbon atoms and the heteroaryl moiety is an aromatic ring system comprising from 5 to 10 atoms including at least one N, O, S, or P.

The term “sulfinyl” as a group or part of a group, refers to the —S(═O)—H group, which can also be written —SO—H.

The term “alkylsulfinyl”, as a group or part of a group, refers to a group of formula —S(═O)—R^{b} wherein R^{b} is alkyl as defined herein.

The term “cycloalkylsulfinyl”, as a group or part of a group, refers to a group of formula —S(═O)—R^{g} wherein R^{g} is cycloalkyl as defined herein.

The term “arylsulfinyl”, as a group or part of a group, refers to a group of formula —S(═O)—R^{f} wherein R^{f} is aryl as defined herein.

The term “mono or di(alkyl)aminosulfinyl”, as a group or part of a group, refers to a group of formula —S(═O)—N(R^{l})(R^{b}), wherein R^{l} is hydrogen or alkyl, R^{b} is alkyl as defined herein.

The term “sulfonyl” as a group or part of a group, refers to the —S(═O)₂H group, which can also be written —SO₂H.

The term “alkylsulfonyl”, as a group or part of a group, refers to a group of formula —S(═O)₂—R^{b} wherein R^{b} is alkyl as defined herein.

The term “cycloalkylsulfonyl”, as a group or part of a group, refers to a group of formula —S(═O)₂—R^{g} wherein R^{g} is cycloalkyl as defined herein.

The term “arylsulfonyl”, as a group or part of a group, refers to a group of formula —S(═O)₂—R^{f}, wherein R^{f} is aryl as defined herein.

The term “mono or di(alkyl)aminosulfonyl”, as a group or part of a group, refers to a group of formula —S(═O)₂—N(R^{l})(R^{b}), wherein R^{l} is hydrogen or alkyl, R^{b} is alkyl as defined herein.

The term “alkoxycarbonylamino” or “alkyloxycarbonylamino”, as a group or part of a group, refers to a group of formula —N(R^{l})—C(═O)—O—R^{b}, wherein R^{l} is hydrogen or alkyl, R^{b} is alkyl as defined herein.

The term “alkenyloxycarbonylamino”, as a group or part of a group, refers to a group of formula —N(R^{l})—C(═O)—O—R^{d}, wherein R^{l} is hydrogen or alkyl, R^{d} is alkenyl as defined herein.

The term “alkynyloxycarbonylamino”, as a group or part of a group, refers to a group of formula —N(R^{l})—C(═O)—O—R^{c}, wherein R^{l} is hydrogen or alkyl, R^{c} is alkynyl as defined herein.

The term “alkylcarbonylamino”, as a group or part of a group, refers to a group of formula —N(R^{l})—C(═O)—R^{b}, wherein R^{l} is hydrogen or alkyl, R^{b} is alkyl as defined herein.

The term “alkenylcarbonylamino”, as a group or part of a group, refers to a group of formula —N(R^{l})—C(═O)—R^{d}, wherein R^{l} is hydrogen or alkyl, R^{d} is alkenyl as defined herein.

The term “alkynylcarbonylamino”, as a group or part of a group, refers to a group of formula —N(R^{l})—C(═O)—R^{c}, wherein R^{l} is hydrogen or alkyl, R^{c} is alkynyl as defined herein.

The term “cycloalkylcarbonylamino”, as a group or part of a group, refers to a group of formula —N(R^{l})—C(═O)—R^{g}, wherein R^{l} is hydrogen or alkyl, R^{g} is cycloalkyl as defined herein.

The term “arylcarbonylamino”, as a group or part of a group, refers to a group of formula —N(Rⁱ)—C(═O)—R^{f}, wherein R^{l} is hydrogen or alkyl, R^{f} is aryl as defined herein.

The term “mono or di(alkyl)aminocarbonyl”, as a group or part of a group, refers to a group of formula —C(═O)—N(R^{l})(R^{b}), wherein R^{l} is hydrogen or alkyl, R^{b} is alkyl as defined herein.

The term “alkylcarbonyloxy”, as a group or part of a group, refers to a group of formula —O—C(═O)—R^{b}, wherein R^{b} is alkyl as defined herein.

The term “alkenylcarbonyloxy”, as a group or part of a group, refers to a group of formula —O—C(═O)—R^{d}, wherein R^{d} is alkenyl as defined herein.

The term “alkynylcarbonyloxy”, as a group or part of a group, refers to a group of formula —O—C(═O)—R^{c}, wherein R^{c} is alkynyl as defined herein.

The term “cycloalkylcarbonyloxy”, as a group or part of a group, refers to a group of formula —O—C(═O)—R^{g}, wherein R^{g} is cycloalkyl as defined herein.

The term “arylcarbonyloxy”, as a group or part of a group, refers to a group of formula —O—C(═O)—R^{f}, wherein R^{f} is aryl as defined herein.

The term “mono or di(alkyl)aminoalkylamino”, as a group or part of a group, refers to a group of formula —N(R^{l})—R^{a}—N(R^{l})(R^{b}), wherein R^{a} is alkylene, R^{l} is hydrogen or alkyl, R^{b} is alkyl as defined herein.

The term “mono or di(alkyl)aminoalkoxy”, as a group or part of a group, refers to a group of formula —O—R^{a}—N(R^{l})(R^{b}), wherein R^{a} is alkylene, R^{l} is hydrogen or alkyl, R^{b} is alkyl as defined herein.

The term “arylamino”, as a group or part of a group, refers to a group of formula —N(R^{l})(R^{f}), wherein R^{l} is hydrogen or alkyl, R^{f} is aryl as defined herein.

The term “arylaminoalkyl”, as a group or part of a group, refers to a group of formula —R^{a}—N(R^{l})(R^{f}), wherein R^{a} is alkylene, R^{l} is hydrogen or alkyl, R^{f} is aryl as defined herein.

The term “alkylcarbonyloxyalkyl”, as a group or part of a group, refers to a group of formula as a group or part of a group, refers to a group of formula —R^{a}—O—C(═O)—R^{b}, wherein R^{a} is alkylene, and R^{b} is alkyl as defined herein.

The term “alkenylcarbonyloxyalkyl”, as a group or part of a group, refers to a group of formula —R^{a}—O—C(═O)—R^{d}, wherein R^{a} is alkylene, and R^{d} is alkenyl as defined herein.

The term “alkynylcarbonyloxyalkyl”, as a group or part of a group, refers to a group of formula —R^{a}—O—C(═O)—R^{c}, wherein R^{a} is alkylene, and R^{c} is alkynyl as defined herein.

The term “arylcarbonyloxy”, as a group or part of a group, refers to a group of formula —O—C(═O)—R^{f}, wherein and R^{f} is aryl as defined herein.

The term “arylcarbonyloxyalkyl”, as a group or part of a group, refers to a group of formula —R^{a}—O—C(═O)—R^{f}, wherein R^{a} is alkylene, and R^{f} is aryl as defined herein

The term “arylaminocarbonyl”, as a group or part of a group, refers to a group of formula —C(═O)—N(R^{l})(R^{f}), wherein R^{l} is hydrogen or alkyl, R^{f} is aryl as defined herein.

The term “heterocyclyloxy”, as a group or part of a group, refers to a group of formula —O—R^{o}, wherein R^{o} is heterocyclyl as defined herein.

The term “heteroaryloxy”, as a group or part of a group, refers to a group of formula —O—R^{p} wherein R^{p} is heteroaryl as defined herein.

The term “heteroarylthio”, as a group or part of a group, refers to a group of formula —S—R^{p} wherein R^{p} is heteroaryl as defined herein.

The term “heteroaryloxyalkyl”, as a group or part of a group, refers to a group of formula —R^{a}—O—R^{p}, wherein R^{a} is alkylene, and R^{p} is heteroaryl as defined herein.

The term “heteroaryloxyalkenyl”, as a group or part of a group, refers to a group of formula —R^{h}—O—R^{p}, wherein R^{h} is alkenylene, and R^{p} is heteroaryl as defined herein.

The term “heteroaryloxyalkynyl”, as a group or part of a group, refers to a group of formula —Rⁱ—O—R^{p}, wherein Rⁱ is alkynylene, and R^{p} is heteroaryl as defined herein.

The term “heteroarylsulfinyl”, as a group or part of a group, refers to a group of formula —S(═O)—R^{p} wherein R^{p} is heteroaryl as defined herein.

The term “heteroarylsulfonyl”, as a group or part of a group, refers to a group of formula —S(═O)₂—R^{p} wherein R^{p} is heteroaryl as defined herein.

The term “heteroarylamino”, as a group or part of a group, refers to a group of formula —N(R^{l})(R^{p}), wherein R^{l} is hydrogen or alkyl, R^{p} is heteroaryl as defined herein.

The term “heteroarylaminoalkyl”, as a group or part of a group, refers to a group of formula —R^{a}—N(R^{l})(R^{p}), wherein R^{a} is alkylene, R^{l} is hydrogen or alkyl, R^{p} is heteroaryl as defined herein.

The term “heteroarylcarbonylamino”, as a group or part of a group, refers to a group of formula —N(R^{l})—C(═O)—R^{p}, wherein R^{l} is hydrogen or alkyl, R^{p} is heteroaryl as defined herein.

The term “heteroarylcarbonyl”, as a group or part of a group, refers to a group of formula —C(═O)—R^{p}, wherein R^{p} is heteroaryl as defined herein.

The term “heteroarylcarbonyloxy”, as a group or part of a group, refers to a group of formula —O—C(═O)—R^{p} wherein R^{p} is heteroaryl as defined herein.

The term “heteroarylcarbonyloxyalkyl”, as a group or part of a group, refers to a group of formula —R^{a}—O—C(═O)—R^{p}, wherein R^{a} is alkylene, R^{p} is heteroaryl as defined herein.

The term “heteroarylaminocarbonyl”, as a group or part of a group, refers to a group of formula —C(═O)—N(R^{l})(R^{p}), wherein R^{l} is hydrogen or alkyl, R^{p} is heteroaryl as defined herein.

The term “single bond” as used herein for a linking group i.e., in a way that a certain linking group is selected from a single bond, etc. in the formulas herein, refers to a molecule wherein the linking group is not present and therefore refers to compounds with a direct linkage via a single bond between the two moieties being linked by the linking group.

The term “double bond” as used herein for a linking group i.e., in a way that a certain linking group is selected from a single bond, etc. in the formulas herein, refers to a molecule wherein the linking group is not present and therefore refers to compounds with a direct linkage via a double bond between the two moieties being linked by the linking group.

The term “triple bond” as used herein for a linking group i.e., in a way that a certain linking group is selected from a single bond, etc. in the formulas herein, refers to a molecule wherein the linking group is not present and therefore refers to compounds with a direct linkage via a triple bond between the two moieties being linked by the linking group.

Any substituent designation that is found in more than one site in a compound of this invention shall be independently selected.

Substituents optionally are designated with or without bonds. Regardless of bond indications, if a substituent is polyvalent (based on its position in the structure referred to), then any and all possible orientations of the substituent are intended.

As used herein and unless otherwise stated, the term “solvate” includes any combination which may be formed by a derivative of this invention with a suitable inorganic solvent (e.g., hydrates) or organic solvent, such as but not limited to alcohols, ketones, esters, ethers, nitriles, and the like.

Preferred statements (features) and embodiments of the methods, compositions, and uses of this invention are set herein below. Each statement and embodiment of the invention so defined may be combined with any other statement and/or embodiment, unless clearly indicated to the contrary. In particular, any feature indicated as being preferred or advantageous may be combined with any other features or statements indicated as being preferred or advantageous.

Hereto, the present invention is in particular captured by any one or any combination of one or more of the below numbered statements and embodiments, with any other aspect and/or embodiment.

1. A compound of formula (I), or a tautomer, a stereoisomer, a hydrate, a solvate, a polymorph, a prodrug, an isotope, or a co-crystal thereof, or a pharmaceutically acceptable salt thereof, wherein

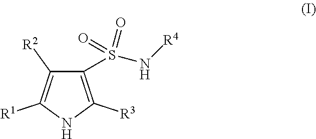

R¹ is selected from the group comprising aryl, heteroaryl, cycloalkyl, cycloalkenyl, cycloalkynyl, heterocyclyl, and A¹-X¹—; and R² is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl;
   wherein each of said aryl, heteroaryl, cycloalkyl, cycloalkenyl, cycloalkynyl, heterocyclyl, X¹ and A¹ of R¹ can be unsubstituted or substituted with one or more Z¹;
X¹ is —Y^{1b}—Y^{1a}—Y^{1c}— wherein Y^{1a} is a single bond, double bond or triple bond or is selected from the group comprising —CR^{1a}═CR^{1a}—, —C≡C—, —CO—, —O—, —CS—, —S—, —SO₂—, —SO—, —SO(NH)—, —CONR^{1b}—, —NR^{1b}CO—, —SO₂NR^{1b}—, —NR^{1b}SO₂—, —S(O)—NR^{1b}—, and —NR^{1b}—;
each of Y^{1b} and Y^{1c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{1a}; wherein when Y^{1a} is a single bond, double bond, or triple bond, at least one of Y^{1b} and Y^{1c} is not a single bond; preferably when Y^{1a} is a triple bond or a double bond, each of Y^{1b} and Y^{1c} is not a single bond, a C₂alkenylene, or a C₂alkynylene;
each R^{1a} is independently selected from the group comprising hydrogen, oxo, thioxo, halo, hydroxy, haloalkyl, alkoxy, alkoxyalkyl, haloalkoxy, haloalkoxyalkyl, mono or di(alkyl)amino, mono or di(alkyl)aminoalkyl, and alkyl;
A¹ is selected from the group comprising aryl, heteroaryl, cycloalkyl, cycloalkenyl, cycloalkynyl, and heterocyclyl;
each Z¹ is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, hydroxyalkyl, alkoxyalkyl, cycloalkyloxy, cycloalkylalkoxy, alkoxyalkoxy, carboxyl, alkoxycarbonyl, alkylcarbonyl, arylalkoxy, amino, mono or di(alkyl)amino, aminoalkyl, mono or di(alkyl)aminoalkyl, mono or di(alkyl)aminocarbonyl, heterocyclyl, heteroaryl, heterocyclylalkyl, heteroarylalkyl, arylalkenyl, arylalkynyl, haloalkenyloxy, haloalkynyloxy, hydroxyalkenyl, hydroxyalkynyl, alkenyloxyalkyl, alkynyloxyalkyl, alkoxyalkenyl, alkoxyalkynyl, alkenyloxyalkoxy, alkynyloxyalkoxy, alkenyloxycarbonyl, alkynyloxycarbonyl, alkenylcarbonyl, alkynylcarbonyl, aminoalkenyl, aminoalkynyl, mono or di(alkyl)aminoalkenyl, mono or di(alkyl)aminoalkynyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{1a};
and/or two Z¹ together with the atom(s) to which they are attached can form an aryl, a cycloalkyl, a heteroaryl, or a heterocyclyl; wherein each of said aryl, cycloalkyl, heteroaryl, and heterocyclyl can be unsubstituted or substituted with one or more Z^{1a};
and/or one R^{1a} together with one Z¹ and the atom(s) to which they are attached can form a cycloalkyl, a 4-10 membered saturated or partially saturated heterocyclyl, a 5-10 membered heteroaryl, or an aryl; wherein each of said cycloalkyl, heterocyclyl, heteroaryl or aryl can be unsubstituted or substituted with one or more Z^{1a};
R^{1b} is hydrogen or alkyl, or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a};
each Z^{1a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
or R¹ is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl; and R² is selected from the group comprising aryl, heteroaryl, cycloalkyl, cycloalkenyl, cycloalkynyl, heterocyclyl, and A²-X²—;
   wherein each of said aryl, heteroaryl, cycloalkyl, cycloalkenyl, cycloalkynyl, heterocyclyl, X² and A² of R², can be unsubstituted or substituted with one or more Z²;
X² is —Y^{2b}—Y^{2a}—Y^{2c}—, wherein Y^{2a} is a single bond, double bond or triple bond or is selected from the group comprising —CR^{2a}═CR^{2a}—, —C═C—, —CO—, —O—, —CS—, —S—, —SO₂—, —SO—, —SO(NH)—, —CONR^{2b}—, —NR^{2b}CO—, —SO₂NR^{2b}—, —NR^{2b}SO²—, —S(O)—NR^{2b}—, and —NR^{2b}—;
each of Y^{2b} and Y^{2c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{2a}; wherein when Y^{2a} is a single bond, double bond, or triple bond, at least one of Y^{2b} and Y^{2e} is not a single bond; preferably when Y^{2a} is a triple bond or a double bond, each of Y^{2b} and Y^{2c} is not a single bond, a C₂alkenylene, or a C₂alkynylene;
each R^{2a} is independently selected from the group comprising hydrogen, oxo, thioxo, halo, hydroxy, haloalkyl, alkoxy, alkoxyalkyl, haloalkoxy, haloalkoxyalkyl, mono or di(alkyl)amino, mono or di(alkyl)aminoalkyl, and alkyl;
A² is selected from the group comprising aryl, heteroaryl, cycloalkyl, cycloalkenyl, cycloalkynyl, and heterocyclyl;
each Z² is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, arylalkenyl, arylalkynyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, haloalkenyloxy, haloalkynyloxy, hydroxyalkyl, hydroxyalkenyl, hydroxyalkynyl, alkoxyalkyl, alkenyloxyalkyl, alkynyloxyalkyl, alkoxyalkenyl, alkoxyalkynyl, cycloalkyloxy, cycloalkylalkoxy, alkoxyalkoxy, alkenyloxyalkoxy, alkynyloxyalkoxy, carboxyl, alkoxycarbonyl, alkenyloxycarbonyl, alkynyloxycarbonyl, alkylcarbonyl, alkenylcarbonyl, alkynylcarbonyl, arylalkoxy, amino, mono or di(alkyl)amino, aminoalkyl, aminoalkenyl, aminoalkynyl, mono or di(alkyl)aminoalkyl, mono or di(alkyl)aminoalkenyl, mono or di(alkyl)aminoalkynyl, mono or di(alkyl)aminocarbonyl, heterocyclyl, heteroaryl, heterocyclylalkyl, heteroarylalkyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, arylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{2a};
and/or two Z² together with the atom(s) to which they are attached can form an aryl, a cycloalkyl, a heteroaryl, or a heterocyclyl; wherein each of said aryl, cycloalkyl, heteroaryl, and heterocyclyl can be unsubstituted or substituted with one or more Z^{2a};
and/or one R^{2a} together with one Z² and the atom(s) to which they are attached can form a cycloalkyl, a 4-10 membered saturated or partially saturated heterocyclyl, a 5-10 membered heteroaryl, or an aryl; wherein each of said cycloalkyl, heterocyclyl, heteroaryl, or aryl can be unsubstituted or substituted with one or more Z^{2a};
R^{2b} is hydrogen or alkyl, or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};
each Z^{2a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
R³ is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl;
R⁴ is aryl, or heteroaryl;
wherein each of said aryl and heteroaryl, is substituted with one or more Z⁴;
each Z⁴ is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, arylalkenyl, arylalkynyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, haloalkenyloxy, haloalkynyloxy, hydroxyalkyl, hydroxyalkenyl, hydroxyalkynyl, alkoxyalkyl, alkenyloxyalkyl, alkynyloxyalkyl, alkoxyalkenyl, alkoxyalkynyl, cycloalkyloxy, cycloalkylalkoxy, alkoxyalkoxy, alkenyloxyalkoxy, alkynyloxyalkoxy, carboxyl, alkoxycarbonyl, alkenyloxycarbonyl, alkynyloxycarbonyl, alkylcarbonyl, alkenylcarbonyl, alkynylcarbonyl, arylalkoxy, amino, mono or di(alkyl)amino, aminoalkyl, aminoalkenyl, aminoalkynyl, mono or di(alkyl)aminoalkyl, mono or di(alkyl)aminoalkenyl, mono or di(alkyl)aminoalkynyl, mono or di(alkyl)aminocarbonyl, heterocyclyl, heteroaryl, heterocyclylalkyl, heteroarylalkyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, arylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{4a};
and/or two Z⁴ together with the atom(s) to which they are attached can form an aryl, a cycloalkyl, a heteroaryl, or a heterocyclyl, wherein each of said aryl, heteroaryl, cycloalkyl, and heterocyclyl can be unsubstituted or substituted with one or more Z^{4a};
each Z^{4a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
with the proviso that
when R¹ is A¹-X¹—, X¹ is —CO—, and A¹ is heterocyclyl, then A¹ is not attached to X¹ via an N ring atom of said heterocyclyl;
when R¹ is a heteroaryl, R¹ is not oxadiazolyl;
when R² is A²-X²—, X² is —CO—, and A² is heterocyclyl, then A² is not attached to X² via an N ring atom of said heterocyclyl; and
when R² is a heteroaryl, R² is not oxadiazolyl;
with the proviso that said compound is not
N,4-bis(4-methylphenyl)-1H-pyrrole-3-sulfonamide; (CAS no 1427286-05-2),
N,4-bis(4-chlorophenyl)-1H-pyrrole-3-sulfonamide (CAS no 1427286-06-3).

2. The compound according to statement 1, wherein
R¹ is selected from the group comprising C₆₋₁₀aryl, 5-10 membered heteroaryl, C₃₋₁₀cycloalkyl, C₅₋₁₀cycloalkenyl, C₅₋₁₀cycloalkynyl, 3-10 membered saturated or partially saturated heterocyclyl, and A¹-X¹—; and R² is selected from the group comprising hydrogen, halo, cyano, C₁₋₆alkyl, C₂₋₆alkenyl, C₂₋₆alkynyl, haloC₁₋₆alkyl, haloC₂₋₆alkenyl, haloC₂₋₆alkynyl, C₁₋₆alkoxy, C₂₋₆alkenyloxy, C₂₋₆alkynyloxy, C₁₋₆alkylthio, C₂₋₆alkenylthio, C₂₋₆alkynylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl;
   wherein each of said C₆₋₁₀aryl, 5-10 membered heteroaryl, C₃₋₁₀cycloalkyl, C₅₋₁₀cycloalkenyl, C₅₋₁₀cycloalkynyl, 3-10 membered saturated or partially saturated heterocyclyl, X¹ and A¹ of R¹, can be unsubstituted or substituted with one or more Z¹;
X¹ is —Y^{1b}—Y^{1a}—Y^{1c}—, wherein Y^{1a} is a single bond, double bond or triple bond or is selected from the group comprising —CR^{1a}═CR^{1a}—, —C═C—, —CO—, —O—, —CS—, —S—, —SO₂—, —SO—, —SO(NH)—, —CONR^{1b}—, —NR^{1b}CO—, —SO₂NR^{1b}—, —NR^{1b}SO₂—, —S(O)—NR^{1b}—, and —NR^{1b}—; preferably X¹ is selected from the group comprising —C(R^{1a})₂—, —CR^{1a}═CR^{1a}—, —C═C—, —CO—, —O—, —CS—, —S—, —SO₂—, —SO—, —SO(NH)—, —CONR^{1b}—, —NR^{1b}CO—, —SO₂NR^{1b}—, —NR^{1b}SO₂—, —S(O)—NR^{1b}—, and —NR^{1b}—; preferably X¹ is selected from the group comprising —C(R^{1a})₂—, —CO—, —O—, —S—, —SO₂—, —SO—, and —NR^{1b}—; preferably X¹ is selected from the group comprising —C(R¹²)₂—, —CO—, —O—, and —NR^{1b}—;
each of Y^{1b} and Y^{1c} is independently selected from the group comprising a single bond, or C₁₋₃ alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{1a}; wherein when Y^{1a} is a single bond, double bond, or triple bond, at least one of Y^{1b} and Y^{1c} is not a single bond; preferably when Y^{1a} is a triple bond or a double bond, each of Y^{1b} and Y^{1c} is not a single bond, a C₂alkenylene, or a C₂alkynylene;
each R^{1a} is independently selected from the group comprising hydrogen, oxo, thioxo, halo, hydroxy, haloC₁₋₆alkyl, C₁₋₆alkoxy, C₁₋₆alkoxyC₁₋₆alkyl, haloC₁₋₆ealkoxy, haloC₁₋₆alkoxyC₁₋₆alkyl, mono or di(C₁₋₆alkyl)amino, mono or di(C₁₋₆alkyl)aminoC₁₋₆alkyl, and C₁₋₆alkyl; preferably each R^{1a} is independently selected from the group comprising hydrogen, halo, hydroxy, haloC₁₋₆alkyl, C₁₋₆alkoxy, C₁₋₆alkoxyC₁₋₆alkyl, haloC₁₋₆alkoxy, haloC₁₋₆alkoxyC₁₋₆alkyl, mono or di(C₁₋₆alkyl)amino, mono or di(C₁₋₆alkyl)aminoC₁₋₆alkyl, and C₁₋₆alkyl;
A¹ is selected from the group comprising C₆₋₁₀aryl, 5-10 membered heteroaryl, C₃₋₁₀cycloalkyl, C₅₋₁₀cycloalkenyl, C₅₋₁₀cycloalkynyl, and 3-10 membered saturated or partially saturated heterocyclyl;
each Z¹ is independently selected from halo, cyano, hydroxy, oxo, nitro, thioxo, or from the group comprising C₁₋₆alkyl, C₂₋₆alkenyl, C₂₋₆alkynyl, C₃₋₁₀cycloalkyl, C₃₋₁₀ocycloalkylC₁₋₆alkyl, C₅₋₁₀cycloalkenyl, C₅₋₁₀cycloalkynyl, C₆₋₁₀aryl, C₆₋₁₀arylC₁₋₆alkyl, haloC₁₋₆alkyl, haloC₂₋₆alkenyl, haloC₂₋₆alkynyl, cyanoC₁₋₆alkyl, C₁₋₆alkoxy, C₂₋₆alkenyloxy, C₂₋₆alkynyloxy, cyanoC₁₋₆alkoxy, C₁₋₆alkylthio, C₂₋₆alkenylthio, C₂₋₆alkynylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀arylC₂₋₆alkenyl, C₆₋₁₀arylC₂₋₆alkynyl, haloC₂₋₆alkenyloxy, haloC₂₋₆alkynyloxy, hydroxyC₂₋₆alkenyl, hydroxyC₂₋₆alkynyl, C₂₋₆alkenyloxyC₁₋₆alkyl, C₂₋₆alkynyloxyC₁₋₆alkyl, C₂₋₆alkenyloxyC₁₋₆alkoxy, C₂₋₆alkynyloxyC₁₋₆alkoxy, C₂₋₆alkenyloxycarbonyl, C₂₋₆alkynyloxycarbonyl, C₂₋₆alkenylcarbonyl, C₂₋₆alkynylcarbonyl, aminoC₂₋₆alkenyl, aminoC₂₋₆alkynyl, mono or di(C₁₋₆alkyl)aminoC₂₋₆alkenyl, mono or di(C₁₋₆alkyl)aminoC₂₋₆alkynyl, 3-10 membered saturated or partially saturated heterocyclylC₂₋₆alkenyl, 3-10 membered saturated or partially saturated heterocyclylC₂₋₆alkynyl, 5-10 membered heteroarylC₂₋₆alkenyl, 5-10 membered heteroarylC₂₋₆alkynyl, C₆₋₁₀aryloxy, C₆₋₁₀aryloxyC₁₋₆alkyl, C₆₋₁₀aryloxyC₂₋₆alkenyl, C₆₋₁₀aryloxyC₂₋₆alkynyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₂₋₆alkenyloxycarbonylamino, C₂₋₆alkynyloxycarbonylamino, C₁₋₆alkylcarbonylamino, C₂₋₆alkenylcarbonylamino, C₂₋₆alkynylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂₋₆alkenylcarbonyloxy, C₂₋₆alkynylcarbonyloxy, C₆₋₁₀arylcarbonyloxy, C₅₋₁₀ cycloalkenylC₁₋₆alkyl, C₅₋₁₀cycloalkynylC₁₋₆alkyl, sulfonyl, sulfinyl, mono or di(C₁₋₆alkyl)aminoC₁₋₆alkylamino, mono or di(C₁₋₆alkyl)aminoC₁₋₆alkoxy, C₆₋₁₀arylamino, C₆₋₁₀arylaminoC₁₋₆alkyl, C₁₋₆alkylcarbonyloxyC₁₋₆alkyl, C₂₋₆alkenylcarbonyloxyC₁₋₆alkyl, C₂₋₆alkynylcarbonyloxyC₁₋₆alkyl, C₆₋₁₀arylcarbonyloxy, C₆₋₁₀arylcarbonyloxyC₁₋₆alkyl, C₆₋₁₀ arylaminocarbonyl, 3-10 membered saturated or partially saturated heterocyclyloxy, 5-10 membered heteroaryloxy, 5-10 membered heteroarylthio, 5-10 membered heteroaryloxyC₁₋₆alkyl, 5-10 membered 5-10 membered heteroaryloxyC₂₋₆alkenyl, 5-10 membered heteroaryloxyC₂₋₆alkynyl, 5-10 membered heteroarylsulfinyl, 5-10 membered heteroarylsulfonyl, 5-10 membered heteroarylamino, 5-10 membered heteroarylaminoC₁₋₆alkyl, 5-10 membered heteroarylcarbonylamino, 5-10 membered heteroarylcarbonyl, 5-10 membered heteroarylcarbonyloxy, 5-10 membered heteroarylcarbonyloxyC₁₋₆alkyl, and 5-10 membered heteroarylaminocarbonyl; each of said group can be unsubstituted or substituted with one or more Z^{1a};
and/or two Z¹ together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, a C₃₋₁₀cycloalkyl, and heterocyclyl can be unsubstituted or substituted with one or more Z^{1a};
and/or one R^{1a} together with one Z¹ and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{1a};
R^{1b} is hydrogen or C₁₋₆alkyl, or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a};
each Z^{1a} is independently selected from the group comprising halo, cyano, hydroxyl, C₁₋₆alkyl, C₂₋₆alkenyl, C₂₋₆alkynyl, haloC₁₋₆alkyl, haloC₂₋₆alkenyl, haloC₂₋₆alkynyl, C₁₋₆alkoxy, C₂₋₆alkenyloxy, C₂₋₆alkynyloxy, C₁₋₆alkylthio, C₂₋₆alkenylthio, C₂₋₆alkynylthio, haloC₁₋₆alkoxy, hydroxyC₁₋₆alkyl, C₁₋₆alkoxyC₁₋₆alkyl, C₃₋₁₀cycloalkyl, C₅₋₁₀cycloalkenyl, C₅₋₁₀cycloalkynyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo;
or R¹ is selected from the group comprising hydrogen, halo, cyano, C₁₋₆alkyl, C₂₋₆alkenyl, C₂₋₆alkynyl, haloC₁₋₆alkyl, haloC₂₋₆alkenyl, haloC₂₋₆alkynyl, C₁₋₆alkoxy, C₂₋₆alkenyloxy, C₂₋₆alkynyloxy, C₁₋₆alkylthio, C₂₋₆alkenylthio, C₂₋₆alkynylthio, haloC₁₋₆alkoxy, C₁₋₆alkoxyC₁₋₆alkyl, mono or di(C₁-alkyl)amino, and mono or di(C₁₋₆alkyl)aminoC₁₋₆alkyl; and R² is selected from the group comprising C₆₋₁₀aryl, 5-10 membered heteroaryl, C₃₋₁₀cycloalkyl, C₅₋₁₀cycloalkenyl, C₅₋₁₀cycloalkynyl, 3-10 membered saturated or partially saturated heterocyclyl, and A²-X²;
   wherein each of said C₅₋₁₀aryl, 5-10 membered heteroaryl, C₃₋₁₀cycloalkyl, C₅₋₁₀cycloalkenyl, C₅₋₁₀cycloalkynyl, 3-10 membered saturated or partially saturated heterocyclyl, X² and A² of R², can be unsubstituted or substituted with one or more Z²;
X² is —Y^{2b}—Y^{2a}—Y^{2c}—, wherein Y^{2a} is a single bond, double bond or triple bond or is selected from the group comprising —CR^{2a}═CR^{2a}—, —C═C—, —CO—, —O—, —CS—, —S—, —SO₂—, —SO—, —SO(NH)—, —CONR^{2b}—, —NR^{2b}CO—, —SO₂NR^{2b}—, —NR^{2b}SO₂—, —S(O)—NR^{2b}—, and —NR^{2b}—; preferably X² is selected from the group comprising —C(R^{2a})₂—, —CR^{2a}═CR^{2a}—, —C≡C—, —CO—, —O—, —CS—, —S—, —SO₂—, —SO—, —SO(NH)—, —CONR^{2b}—, —NR^{2b}CO—, —SO₂NR^{2b}—, —NR^{2b}SO₂—, —S(O)—NR^{2b}—, and —NR^{2b}—; preferably X² is selected from —C(R^{2a})₂—, —CO—, —O—, —S—, —SO₂—, —SO—, or —NR^{2b}—; preferably X² is selected from —C(R^{2a})₂—, —CO—, —O—, or —NR^{2b};
each of Y^{2b} and Y^{2c} is independently selected from the group comprising a single bond, or C₁₋₃ alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{2a}; wherein when Y^{2a} is a single bond, double bond, or triple bond, at least one of Y^{2b} and Y^{2c} is not a single bond;
preferably when Y^{2a} is a triple bond or a double bond, each of Y^{2b} and Y^{2c} is not a single bond, a C₂alkenylene, or a C₂alkynylene;
each R^{2a} is independently selected from the group comprising hydrogen, oxo, thioxo, halo, hydroxy, haloC₁₋₆alkyl, C₁₋₆alkoxy, C₁₋₆alkoxyC₁₋₆alkyl, haloC₁₋₆alkoxy, haloC₁₋₆alkoxyC₁₋₆alkyl, mono or di(C₁₋₆alkyl)amino, mono or di(C₁₋₆alkyl)aminoC₁₋₆alkyl, and C₁₋₆alkyl; preferably each R^{2a} is independently selected from the group comprising hydrogen, halo, hydroxy, haloC₁₋₆alkyl, C₁₋₆alkoxy, C₁₋₆alkoxyC₁₋₆alkyl, haloC₁₋₆alkoxy, haloC₁₋₆alkoxyC₁₋₆alkyl, mono or di(C₁₋₆alkyl)amino, mono or di(C₁₋₆alkyl)aminoC₁₋₆alkyl, and C₁₋₆alkyl;
A² is selected from the group comprising C₆₋₁₀aryl, 5-10 membered heteroaryl, C₃₋₁₀cycloalkyl, C₅₋₁₀cycloalkenyl, C₅₋₁₀cycloalkynyl, and 3-10 membered saturated or partially saturated heterocyclyl;
each Z² is independently selected from halo, cyano, hydroxy, oxo, nitro, thioxo, or from the group comprising C₁₋₆alkyl, C₂₋₆alkenyl, C₂₋₆alkynyl, C₃₋₁₀cycloalkyl, C₃₋₁₀cycloalkylC₁₋₆alkyl, C₅₋₁₀cycloalkenyl, C₅₋₁₀cycloalkynyl, C₁₋₆aryl, C₆₋₁₀arylC₁₋₆alkyl, haloC₁₋₆alkyl, haloC₂₋₆alkenyl, haloC₂₋₆alkynyl, cyanoC₁₋₆alkyl, C₁₋₆alkoxy, C₂₋₆alkenyloxy, C₂₋₆alkynyloxy, cyanoC₁₋₆alkoxy, C₁₋₆alkylthio, C₂₋₆alkenylthio, C₂₋₆alkynylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀arylC₂₋₆alkenyl, C₆₋₁₀arylC₂₋₆alkynyl, haloC₂₋₆alkenyloxy, haloC₂₋₆alkynyloxy, hydroxyC₂₋₆alkenyl, hydroxyC₂₋₆alkynyl, C₂₋₆alkenyloxyC₁₋₆alkyl, C₂₋₆alkynyloxyC₁₋₆alkyl, C₂₋₆alkenyloxyC₁₋₆alkoxy, C₂₋₆alkynyloxyC₁₋₆alkoxy, C₂₋₆alkenyloxycarbonyl, C₂₋₆alkynyloxycarbonyl, C₂₋₆alkenylcarbonyl, C₂₋₆alkynylcarbonyl, aminoC₂₋₆alkenyl, aminoC₂₋₆alkynyl, mono or di(C₁₋₆alkyl)aminoC₂₋₆alkenyl, mono or di(C₁₋₆alkyl)aminoC₂₋₆alkynyl, 3-10 membered saturated or partially saturated heterocyclylC₂₋₆alkenyl, 3-10 membered saturated or partially saturated heterocyclylC₂₋₆alkynyl, 5-10 membered heteroarylC₂₋₆alkenyl, 5-10 membered heteroarylC₂₋₆alkynyl, C₆₋₁₀aryloxy, C₆₋₁₀ aryloxyC₁₋₆alkyl, C₆₋₁₀aryloxyC₂₋₆alkenyl, C₆₋₁₀aryloxyC₂₋₆alkynyl, C₆₋₁₀arylthio, haloC₁₋₆alkythio, C₃₋₁₀cycloalkylthio, C₁₋₆alkylsulfinyl, C₁-alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₂₋₆alkenyloxycarbonylamino, C₂₋₆alkynyloxycarbonylamino, C₁₋₆alkylcarbonylamino, C₂₋₆alkenylcarbonylamino, C₂₋₆alkynylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂₋₆alkenylcarbonyloxy, C₂₋₆alkynylcarbonyloxy, C₆₋₁₀arylcarbonyloxy, C₅₋₁₀cycloalkenylC₁₋₆alkyl, C₅₋₁₀cycloalkynylC₁₋₆alkyl, sulfonyl, sulfinyl, mono or di(C₁₋₆alkyl)aminoC₁₋₆alkylamino, mono or di(C₁₋₆alkyl)aminoC₁₋₆alkoxy, C₆₋₁₀arylamino, C₆₋₁₀arylaminoC₁₋₆alkyl, C₁₋₆alkylcarbonyloxyC₁₋₆alkyl, C₂₋₆alkenylcarbonyloxyC₁₋₆alkyl, C₂₋₆alkynylcarbonyloxyC₁₋₆alkyl, C₆₋₁₀arylcarbonyloxy, C₆₋₁₀arylcarbonyloxyC₁₋₆alkyl, C₆₋₁₀arylaminocarbonyl, 3-10 membered saturated or partially saturated heterocyclyloxy, 5-10 membered heteroaryloxy, 5-10 membered heteroarylthio, 5-10 membered heteroaryloxyC₁₋₆alkyl, 5-10 membered 5-10 membered heteroaryloxyC₂₋₆alkenyl, 5-10 membered heteroaryloxyC₂₋₆alkynyl, 5-10 membered heteroarylsulfinyl, 5-10 membered heteroarylsulfonyl, 5-10 membered heteroarylamino, 5-10 membered heteroarylaminoC₁₋₆alkyl, 5-10 membered heteroarylcarbonylamino, 5-10 membered heteroarylcarbonyl, 5-10 membered heteroarylcarbonyloxy, 5-10 membered heteroarylcarbonyloxyC₁₋₆alkyl, and 5-10 membered heteroarylaminocarbonyl; each of said group can be unsubstituted or substituted with one or more Z^{2a};
and/or two Z² together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, a C₃₋₁₀cycloalkyl, and heterocyclyl can be unsubstituted or substituted with one or more Z^{2a};
and/or one R^{2a} together with one Z² and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{2a};
R^{2b} is hydrogen or C₁₋₆alkyl, or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};
each Z^{2a} is independently selected from the group comprising halo, cyano, hydroxyl, C₁₋₆alkyl, C₂₋₆alkenyl, C₂₋₆alkynyl, haloC₁₋₆alkyl, haloC₂₋₆alkenyl, haloC₂₋₆alkynyl, C₁₋₆alkoxy, C₂₋₆alkenyloxy, C₂₋₆alkynyloxy, C₁₋₆alkylthio, C₂₋₆alkenylthio, C₂₋₆alkynylthio, haloC₁₋₆alkoxy, hydroxyC₁₋₆alkyl, C₁₋₆alkoxyC₁₋₆alkyl, C₃₋₁₀cycloalkyl, C₅₋₁₀cycloalkenyl, C₅₋₁₀cycloalkynyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo;
R³ is selected from the group comprising hydrogen, halo, cyano, C₁₋₆alkyl, C₂₋₆alkenyl, C₂₋₆alkynyl, haloC₁₋₆alkyl, haloC₂₋₆alkenyl, haloC₂₋₆alkynyl, C₁₋₆alkoxy, C₂₋₆alkenyloxy, C₂₋₆alkynyloxy, C₁₋₆alkylthio, C₂₋₆alkenylthio, C₂₋₆alkynylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl;
R⁴ is C₆₋₁₀aryl, or 5-10 membered heteroaryl;
wherein each of said C₆₋₁₀aryl and 5-10 membered heteroaryl, is substituted with one or more Z⁴;
each Z⁴ is independently selected from halo, cyano, hydroxyl, oxo, nitro, thioxo, or from the group comprising C₁₋₆alkyl, C₂₋₆alkenyl, C₂₋₆alkynyl, C₃₋₁₀cycloalkyl, C₃₋₁₀cycloalkylC₁₋₆alkyl, C₅₋₁₀cycloalkenyl, C₅₋₁₀cycloalkynyl, C₆₋₁₀aryl, C₆₋₁₀arylC₁₋₆alkyl, haloC₁₋₆alkyl, haloC₂₋₆alkenyl, haloC₂₋₆alkynyl, cyanoC₁₋₆alkyl, C₁₋₆alkoxy, C₂₋₆alkenyloxy, C₂₋₆alkynyloxy, cyanoC₁₋₆alkoxy, C₁₋₆alkylthio, C₂₋₆alkenylthio, C₂₋₆alkynylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀arylC₂₋₆alkenyl, C₆₋₁₀arylC₂₋₆alkynyl, haloC₂₋₆alkenyloxy, haloC₂₋₆alkynyloxy, hydroxyC₂₋₆alkenyl, hydroxyC₂₋₆alkynyl, C₂₋₆alkenyloxyC₁₋₆alkyl, C₂₋₆alkynyloxyC₁₋₆alkyl, C₂₋₆alkenyloxyC₁₋₆alkoxy, C₂₋₆alkynyloxyC₁₋₆alkoxy, C₂₋₆alkenyloxycarbonyl, C₂₋₆alkynyloxycarbonyl, C₂₋₆alkenylcarbonyl, C₂₋₆alkynylcarbonyl, aminoC₂₋₆alkenyl, aminoC₂₋₆alkynyl, mono or di(C₁₋₆alkyl)aminoC₂₋₆alkenyl, mono or di(C₁₋₆alkyl)aminoC₂₋₆alkynyl, 3-10 membered saturated or partially saturated heterocyclylC₂₋₆alkenyl, 3-10 membered saturated or partially saturated heterocyclylC₂₋₆alkynyl, 5-10 membered heteroarylC₂₋₆alkenyl, 5-10 membered heteroarylC₂₋₆alkynyl, C₆₋₁₀aryloxy, C₆₋₁₀aryloxyC₁₋₆alkyl, C₆₋₁₀aryloxyC₂₋₆alkenyl, C₆₋₁₀aryloxyC₂₋₆alkynyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₂₋₆alkenyloxycarbonylamino, C₂₋₆alkynyloxycarbonylamino, C₁₋₆alkylcarbonylamino, C₂₋₆alkenylcarbonylamino, C₂₋₆alkynylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂₋₆alkenylcarbonyloxy, C₂₋₆alkynylcarbonyloxy, C₆₋₁₀arylcarbonyloxy, C₅₋₁₀cycloalkenylC₁₋₆alkyl, C₅₋₁₀cycloalkynylC₁₋₆alkyl, sulfonyl, sulfinyl, mono or di(C₁₋₆alkyl)aminoC₁₋₆alkylamino, mono or di(C₁₋₆alkyl)aminoC₁₋₆alkoxy, C₆₋₁₀arylamino, C₆₋₁₀arylaminoC₁₋₆alkyl, C₁₋₆alkylcarbonyloxyC₁₋₆alkyl, C₂₋₆alkenylcarbonyloxyC₁₋₆alkyl, C₂₋₆alkynylcarbonyloxyC₁₋₆alkyl, C₆₋₁₀arylcarbonyloxy, C₆₋₁₀arylcarbonyloxyC₁₋₆alkyl, C₆₋₁₀arylaminocarbonyl, 3-10 membered saturated or partially saturated heterocyclyloxy, 5-10 membered heteroaryloxy, 5-10 membered heteroarylthio, 5-10 membered heteroaryloxyC₁. 6alkyl, 5-10 membered 5-10 membered heteroaryloxyC₂₋₆alkenyl, 5-10 membered heteroaryloxyC₂₋₆alkynyl, 5-10 membered heteroarylsulfinyl, 5-10 membered heteroarylsulfonyl, 5-10 membered heteroarylamino, 5-10 membered heteroarylaminoC₁₋₆alkyl, 5-10 membered heteroarylcarbonylamino, 5-10 membered heteroarylcarbonyl, 5-10 membered heteroarylcarbonyloxy, 5-10 membered heteroarylcarbonyloxyC₁₋₆alkyl, and 5-10 membered heteroarylaminocarbonyl; each of said group can be unsubstituted or substituted with one or more Z^{4a};
and/or two Z⁴ together with the atom(s) to which they are attached can form an C₆₋₁₀aryl, a 5-10 membered heteroaryl, a C₃₋₁₀cycloalkyl, or a 3-10 membered saturated or partially saturated heterocyclyl, wherein each of said C₆₋₁₀aryl, heteroaryl, C₃₋₁₀cycloalkyl, and heterocyclyl can be unsubstituted or substituted with one or more Z^{4a};
each Z^{4a} is independently selected from the group comprising halo, cyano, hydroxyl, C₁₋₆alkyl, C₂₋₆alkenyl, C₂₋₆alkynyl, haloC₁₋₆alkyl, haloC₂₋₆alkenyl, haloC₂₋₆alkynyl, C₁₋₆alkoxy, C₂₋₆alkenyloxy, C₂₋₆alkynyloxy, C₁₋₆alkylthio, C₂₋₆alkenylthio, C₂₋₆alkynylthio, haloC₁₋₆alkoxy, hydroxyC₁₋₆alkyl, C₁₋₆alkoxyC₁₋₆alkyl, C₃₋₁₀cycloalkyl, C₅₋₁₀cycloalkenyl, C₅₋₁₀cycloalkynyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

3. The compound according to any one of statements 1 or 2, wherein
R¹ is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl and A¹-X¹—, preferably R¹ is selected from the group comprising C₆₋₁₀aryl, 5-10 membered heteroaryl, C₃₋₁₀cycloalkyl, C₅₋₁₀cycloalkenyl, and A¹-X¹—; preferably R¹ is selected from the group comprising C₆₋₁₀aryl, 5-8 membered heteroaryl, C₅₋₁₀cycloalkyl, C₃₋₈cycloalkenyl; and A¹-X¹—; preferably R¹ is selected from the group comprising phenyl, 5-6 membered heteroaryl, C₃₋₆cycloalkyl, C₅₋₆cycloalkenyl; and A¹-X¹—; preferably R¹ is selected from the group comprising phenyl, 5-6 membered heteroaryl, C₄₋₅cycloalkyl, cyclohexenyl; and A¹-X¹—; preferably R¹ is selected from the group comprising phenyl, 5-6 membered heteroaryl, C₄₋₅cycloalkyl, and cyclohexenyl;
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X¹ and A¹ of R¹, can be unsubstituted or substituted with one or more Z¹; and
R² is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; preferably R² is selected from hydrogen, or C₁₋₆alkyl; preferably R² is selected from hydrogen, or C₁₋₄alkyl; preferably R² is selected from hydrogen, or C₁₋₂alkyl; preferably R² is selected from hydrogen, or methyl, preferably R² is hydrogen.

4. The compound according to any one of statements 1-3, wherein
X¹ is —Y^{1b}—Y^{1a}—Y^{1c}— wherein Y^{1a} is a single bond, double bond or triple bond or is selected from the group comprising —CR^{1a}═CR^{1a}—, —C═C—, —CO—, —O—, —CS—, —S—, —SO₂—, —SO—, —SO(NH)—, —CONR^{1b}—, —NR^{1b}CO—, —SO₂NR^{1b}—, —NR^{1b}SO₂—, —S(O)—NR^{1b}—, and —NR^{1b}—; preferably X¹ is selected from the group comprising —C(R^{1a})₂—, —CR^{1a}═CR^{1a}—, —C═C—, —CO—, —O—, —CS—, —S—, —SO₂—, —SO—, —SO(NH)—, —CONR^{1b}—, —NR^{1b}CO—, —SO₂NR^{1b}—, —NR^{1b}SO₂—, —S(O)—NR^{1b}—, and —NR^{1b}—; preferably X¹ is selected from the group comprising —C(R^{1a})₂—, —CO—, —O—, —CS—, —S—, —SO₂—, —SO—, and —NR^{1b}—; preferably X¹ is selected from —C(R^{1a})₂—, —CO—, —O—, or —NR^{1b}—; preferably X¹ is —C(R^{1a})₂—, —CO—, or —NR^{1b}—; preferably X¹ is —C(R^{1a})₂—, or —CO—; preferably X¹ is —C(R^{1a})₂—; preferably X¹ is —CH₂—;
each of Y^{1b} and Y^{1c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{1a}; wherein when Y^{1a} is a single bond, double bond, or triple bond, at least one of Y^{1b} and Y^{1c} is not a single bond; preferably when Y^{1a} is a triple bond or a double bond, each of Y^{1b} and Y^{1c} is not a single bond, a C₂alkenylene, or a C₂alkynylene;
each R^{1a} is independently selected from the group comprising hydrogen, oxo, thioxo, halo, hydroxy, haloC₁₋₆alkyl, C₁₋₆alkoxy, C₁₋alkoxyC₁₋₆alkyl, haloC₁₋₆alkoxy, haloC₁₋₆alkoxyC₁₋₆alkyl, mono or di(C₁₋₆alkyl)amino, mono or di(C₁₋₆alkyl)aminoC₁₋₆alkyl, and C₁₋₆alkyl; preferably each R^{1a} is independently selected from the group comprising hydrogen, halo, hydroxy, haloC₁₋₆alkyl, C₁₋₆alkoxy, haloC₁₋₆alkoxy, and C₁₋₆alkyl; preferably each R^{1a} is independently selected from the group comprising hydrogen, halo, hydroxy, and C₁₋₆alkyl; preferably each R^{1a} is independently selected from hydrogen, or C₁₋₆alkyl; preferably each R^{1a} is independently selected from hydrogen, or C₁₋₄alkyl; preferably each R^{1a} is independently selected from hydrogen, or C₁₋₂alkyl; preferably each R^{1a} is independently selected from hydrogen, or methyl;
A¹ is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl; preferably A¹ is selected from the group comprising C₆₋₁₀aryl, 5-10 membered heteroaryl, C₃₋₁₀cycloalkyl, and C₅₋₁₀cycloalkenyl; preferably A¹ is selected from the group comprising C₆₋₁₀aryl, 5-10 membered heteroaryl, C₃₋₆cycloalkyl, and C₅₋₁₀cycloalkenyl; preferably A¹ is selected from the group comprising C₆₋₁₀aryl, 5-8 membered heteroaryl, C₃₋₆cycloalkyl, and C₅₋₁₀cycloalkenyl; preferably A¹ is selected from the group comprising phenyl, C₃₋₆cycloalkyl, 5-6 membered heteroaryl, and cyclohexenyl; preferably A¹ is selected from phenyl, C₃₋₄cycloalkyl, or 5-6 membered heteroaryl;
and/or one R^{1a} together with one Z¹ and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{1a};
R^{1b} is hydrogen or C₁₋₆alkyl; preferably each R^{1b} is independently selected from hydrogen, or C₁₋₄alkyl; preferably each R^{1b} is independently selected from hydrogen, or C₁₋₂alkyl; preferably each R^{1b} is independently selected from hydrogen, or methyl; or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a}.

5. The compound according to any one of statements 1-4, wherein
each Z¹ is independently selected from halo, cyano, hydroxy, oxo, nitro, thioxo, or from the group comprising C₁₋₆alkyl, C₂₋₆alkenyl, C₃₋₁₀cycloalkyl, C₃₋₁₀cycloalkylC₁₋₆alkyl, C₅₋₁₀cycloalkenyl, C₆₋₁₀aryl, C₆₋₁₀arylC₁₋₆alkyl, haloC₁₋₆alkyl, haloC₂₋₆alkenyl, cyanoC₁₋₆alkyl, C₁₋₆alkoxy, C₂₋₆alkenyloxy, cyanoC₁₋₆alkoxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆ alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀arylC₂₋₆alkenyl, haloC₂₋₆alkenyloxy, hydroxyC₂₋₆alkenyl, C₂₋₆alkenyloxyC₁₋₆alkyl, C₂₋₆alkenyloxyC₁₋₆alkoxy, C₂₋₆alkenyloxycarbonyl, C₂₋₆alkenylcarbonyl, aminoC₂₋₆alkenyl, mono or di(C₁₋₆alkyl)aminoC₂₋₆alkenyl, 3-10 membered saturated or partially saturated heterocyclylC₂₋₆alkenyl, 5-10 membered heteroarylC₂₋₆alkenyl, C₆₋₁₀aryloxy, C₆₋₁₀aryloxyC₁₋₆alkyl, C₆₋₁₀aryloxyC₂₋₆alkenyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₂₋₆alkenyloxycarbonylamino, C₁₋₆alkylcarbonylamino, C₂₋₆alkenylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂₋₆alkenylcarbonyloxy, and C₆₋₁₀arylcarbonyloxy; each of said group can be unsubstituted or substituted with one or more Z^{1a}; preferably each Z¹ is independently selected from halo, cyano, oxo, thioxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀aryloxy, C₆₋₁₀aryloxyC₁₋₆alkyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₁₋₆alkylcarbonylamino, C₆₋₁₀cycloalkylcarbonylamino, C₁₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, and C₆₋₁₀arylcarbonyloxy; each of said group can be unsubstituted or substituted with one or more Z^{1a}; preferably each Z¹ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, wherein each of said group can be unsubstituted or substituted with one or more Z^{1a}; preferably each Z¹ is independently selected from halo, cyano, oxo, or from the group comprising C₁₋₆alkyl, C₃₋₁₀cycloalkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, and C₃₋₁₀cycloalkylC₁₋₆alkoxy, wherein each of said group can be unsubstituted or substituted with one or more Z^{1a}; preferably each Z¹ is independently selected from halo, cyano, oxo, or from the group comprising C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆ alkyl, C₃₋₁₀cycloalkyloxy, and C₃₋₁₀cycloalkylC₁₋₆alkoxy, wherein each of said group can be unsubstituted or substituted with one or more Z^{1a}; preferably each Z¹ is independently selected from halo, cyano, oxo, or from the group comprising C₁₋₄alkyl, haloC₁₋₄alkyl, C₁₋₄alkoxy, C₁₋₄alkylthio, haloC₁₋₄alkoxy, hydroxyC₁₋₄alkyl, C₁₋₄alkoxyC₁₋₄alkyl, C₃₋₆cycloalkyloxy, and C₃₋₆cycloalkylC₁₋₄alkoxy, wherein each of said group can be unsubstituted or substituted with one or more Z^{1a}; preferably each Z¹ is independently selected from halo, cyano, oxo, or from the group comprising C₁₋₂alkyl, haloC₁₋₂ alkyl, C₁₋₂alkoxy, C₁₋₂alkylthio, haloC₁₋₂alkoxy, hydroxyC₁₋₂alkyl, C₁₋₂alkoxyC₁₋₂alkyl, C₃₋₆cycloalkyloxy, and C₃₋₆cycloalkylC₁₋₂alkoxy, wherein each of said group can be unsubstituted or substituted with one or more Z^{1a};
and/or two Z¹ together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{1a}; preferably and/or two Z¹ together with the atom(s) to which they are attached can form a C₆₋₁₀aryl, or a 5-10 membered heteroaryl; wherein each of said C₆₋₁₀aryl and heteroaryl, can be unsubstituted or substituted with one or more Z^{1a}; preferably and/or two Z¹ together with the atom(s) to which they are attached can form a C₆₋₁₀aryl, or a 5-8 membered heteroaryl; wherein each of said C₆₋₁₀aryl and heteroaryl, can be unsubstituted or substituted with one or more Z^{1a}; preferably and/or two Z¹ together with the atom(s) to which they are attached can form a phenyl, or a 5-6 membered heteroaryl; wherein each of said phenyl, and heteroaryl, can be unsubstituted or substituted with one or more Z^{1a};
each Z^{1a} is independently selected from the group comprising halo, cyano, hydroxyl, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁-alkoxy, hydroxyC₁₋₆alkyl, C₁₋₆alkoxyC₁₋₆alkyl, C₃₋₁₀cycloalkyl, C₅₋₁₀ocycloalkenyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; preferably each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; preferably each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀ cycloalkyloxy, and oxo; preferably each Z^{1a} is independently selected from the group comprising halo, cyano, hydroxyl, C₁₋₆alkyl, haloC₁₋₆alkyl, C₁₋₆alkoxy, haloC₁₋₆alkoxy, hydroxyC₁₋₆alkyl, and oxo.

6. The compound according to any one of statements 1 or 2, wherein
R² is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl and A²-X²—; preferably R² is selected from the group comprising C₆₋₁₀aryl, 5-10 membered heteroaryl, C₃₋₁₀cycloalkyl, C₅₋₁₀cycloalkenyl, and A²-X²—, preferably R² is selected from the group comprising C₆₋₁₀aryl, 5-8 membered heteroaryl, C₃₋₈cycloalkyl, C₅₋₈cycloalkenyl, and A²-X²—; preferably R² is selected from the group comprising phenyl, 5-6 membered heteroaryl, C₃₋₆cycloalkyl, C₅₋₆cycloalkenyl, and A²-X²—; preferably R² is selected from the group comprising phenyl, 5-6 membered heteroaryl, C₅₋₆cycloalkyl, C₅₋₆cycloalkenyl, and A²-X²—; preferably R² is selected from the group comprising phenyl, 5-6 membered heteroaryl, cyclopentenyl, and A²-X²—; preferably R² is selected from phenyl, or A²-X²—; preferably R² is A²-X²—; preferably wherein the 5-6 membered heteroaryl is selected from the group comprising pyridyl, pyrrolyl, pyrazinyl, pyridazinyl, pyrimidinyl, thiophenyl, furanyl, thiazolyl, isothiazolyl, and 1,2,5-thiadiazolyl,
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X² and A² of R², can be unsubstituted or substituted with one or more Z²; and
R¹ is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; preferably R¹ is selected from the group comprising hydrogen, halo, cyano, C₁₋₆alkyl, haloC₁₋₆alkyl, C₁₋₆alkoxy, C₁₋₆alkylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; preferably R¹ is selected from the group comprising hydrogen, halo, cyano, C₁₋₆alkyl, haloC₁₋₆alkyl, and C₁₋₆alkoxy; preferably R¹ is selected from hydrogen, or C₁₋₆alkyl; preferably R¹ is selected from hydrogen, or C₁₋₄alkyl; preferably R¹ is selected from hydrogen, or C₁₋₂alkyl; preferably R¹ is selected from hydrogen, or methyl, preferably R¹ is hydrogen.

7. The compound according to any one of statements 1-2,6, wherein
X² is —Y^{2b}—Y^{2a}—Y^{2c}—, wherein Y^{2a} is a single bond, double bond or triple bond or is selected from the group comprising —CR^{2a}═CR^{2a}—, —C≡C—, —CO—, —O—, —CS—, —S—, —SO₂—, —SO—, —SO(NH)—, —CONR^{2b}—, —NR^{2b}CO—, —SO₂NR^{2b}—, —NR^{2b}SO₂—, —S(O)—NR^{2b}—, and —NR^{2b}—; preferably X² is selected from the group comprising —C(R^{2a})₂—, —CR^{2a}═CR^{2a}—, —C═C—, —CO—, —O—, —CS—, —S—, —SO₂—, —SO—, —SO(NH)—, —CONR^{2b}—, —NR^{2b}CO—, —SO₂NR^{2b}—, —NR^{2b}SO₂—, —S(O)—NR^{2b}—, and —NR^{2b}—; preferably X² is selected from the group comprising —C(R^{2a})₂—, —CO—, —O—, —S—, —SO₂—, —SO—, and —NR^{2b}—; preferably X² is selected from —C(R^{2a})₂—, —CO—, —O—, or —NR^{2b}—; preferably X² is —C(R^{2a})₂—, —CO—, or —NR^{2b}—; preferably X² is —C(R^{2a})₂—, or —CO—; preferably X² is —C(R^{2a})₂—; preferably X¹ is —CH₂—;
each of Y^{2b} and Y^{2c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{2a}; wherein when Y^{2a} is a single bond, double bond, or triple bond, at least one of Y^{2b} and Y^{2c} is not a single bond; preferably when Y^{2a} is a triple bond or a double bond, each of Y^{2b} and Y^{2c} is not a single bond, a C₂alkenylene, or a C₂alkynylene;
each R^{2a} is independently selected from the group comprising hydrogen, oxo, thioxo, halo, hydroxy, haloC₁₋₆alkyl, C₁₋₆alkoxy, C₁₋₆alkoxyC₁₋₆alkyl, haloC₁₋₆alkoxy, haloC₁₋₆alkoxyC₁₋₆alkyl, mono or di(C₁₋₆alkyl)amino, mono or di(C₁₋₆alkyl)aminoC₁._alkyl, and C₁₋₆alkyl; preferably each R^{2a} is independently selected from the group comprising hydrogen, halo, hydroxy, haloC₁₋₆ alkyl, C₁₋₆alkoxy, haloC₁₋₆alkoxy, and C₁₋₆alkyl; preferably each R^{2a} is independently selected from the group comprising hydrogen, halo, hydroxy and C₁₋₆alkyl; preferably each R^{2a} is independently selected from hydrogen, hydroxyl, or C₁₋₆alkyl; preferably each R^{2a} is independently selected from hydrogen, hydroxyl or C₁₋₄alkyl; preferably each R^{2a} is independently selected from hydrogen, hydroxyl or C₁₋₂alkyl; preferably each R^{2a} is independently selected from hydrogen, hydroxyl, or methyl;
A² is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl; preferably A² is selected from the group comprising C₆₋₁₀aryl, 5-10 membered heteroaryl, C₃₋₁₀cycloalkyl, and C₅₋₁₀cycloalkenyl; preferably A² is selected from the group comprising C₆₋₁₀aryl, 5-10 membered heteroaryl, and C₅₋₁₀cycloalkenyl; preferably A² is selected from the group comprising C₆₋₁₀aryl, 5-8 membered heteroaryl, C₃₋₆cycloalkyl, and C₅₋₆cycloalkenyl; preferably A² is selected from the group comprising phenyl, 5-6 membered heteroaryl, C₃₋₆cycloalkyl, and C₅₋₁₀cycloalkenyl; preferably A² is selected from the group comprising phenyl, 5-6 membered heteroaryl, and cyclohexenyl; preferably A² is selected from phenyl, or 5-6 membered heteroaryl; preferably A² is phenyl,
and/or one R^{2a} together with one Z² and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{2a};
R^{2b} is hydrogen or C₁₋₆alkyl, preferably each R^{2b} is independently selected from hydrogen, or C₁₋₄alkyl; preferably each R^{2b} is independently selected from hydrogen, or C₁₋₂alkyl; preferably each R^{2b} is independently selected from hydrogen, or methyl; or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};

8. The compound according to any one of statements 1-2, 6-7, wherein each Z² is independently selected from halo, cyano, hydroxy, oxo, nitro, thioxo, or from the group comprising C₁₋₆alkyl, C₂₋₆alkenyl, C₃₋₁₀cycloalkyl, C₃₋₁₀cycloalkylC₁₋₆alkyl, C₁₋₁₀cycloalkenyl, C₆₋₁₀aryl, C₆₋₁₀arylC₁₋₆alkyl, haloC₁₋₆alkyl, haloC₂₋₆alkenyl, cyanoC₁₋₆alkyl, C₁₋₆alkoxy, C₂₋₆alkenyloxy, cyanoC₁₋₆alkoxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆-alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀arylC₂₋₆alkenyl, haloC₂₋₆alkenyloxy, hydroxyC₂₋₆alkenyl, C₂₋₆alkenyloxyC₁₋₆alkyl, C₂₋₆alkenyloxyC₁₋₆alkoxy, C₂₋₆alkenyloxycarbonyl, C₂₋₆alkenylcarbonyl, aminoC₂₋₆alkenyl, mono or di(C₁₋₆alkyl)aminoC₂₋₆alkenyl, 3-10 membered saturated or partially saturated heterocyclylC₂₋₆alkenyl, 5-10 membered heteroarylC₂₋₆alkenyl, C₆₋₁₀aryloxy, C₆₋₁₀aryloxyC₁₋₆alkyl, C₆₋₁₀aryloxyC₂₋₆alkenyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁-alkoxycarbonylamino, C₂₋₆alkenyloxycarbonylamino, C₁-alkylcarbonylamino, C₂₋₆alkenylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂₋₆alkenylcarbonyloxy, and C₆₋₁₀arylcarbonyloxy; each of said group can be unsubstituted or substituted with one or more Z^{2a}; preferably each Z² is independently selected from halo, cyano, oxo, thioxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀aryloxy, C₆₋₁₀aryloxyC₁₋₆alkyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₁₋₆alkylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, and C₆₋₁₀arylcarbonyloxy; each of said group can be unsubstituted or substituted with one or more Z^{2a}; preferably each Z² is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, wherein each of said group can be unsubstituted or substituted with one or more Z^{2a}; preferably each Z² is independently selected from halo, cyano, oxo, or from the group comprising C₁₋₆alkyl, C₃₋₁₀cycloalkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, and C₃₋₁₀ocycloalkylC₁₋₆alkoxy, wherein each of said group can be unsubstituted or substituted with one or more Z^{2a}; preferably each Z² is independently selected from halo, cyano, oxo, or from the group comprising C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, and C₃₋₁₀cycloalkylC₁₋₆alkoxy, wherein each of said group can be unsubstituted or substituted with one or more Z^{2a}preferably each Z² is independently selected from halo, cyano, oxo, or from the group comprising C₁₋₄alkyl, haloC₁₋₄alkyl, C₁₋₄alkoxy, C₁₋₄alkylthio, haloC₁₋₄alkoxy, hydroxyC₁₋₄alkyl, C₁₋₄alkoxyC₁₋₄alkyl, C₃₋₆cycloalkyloxy, and C₃₋₆cycloalkylC₁₋₄alkoxy, wherein each of said group can be unsubstituted or substituted with one or more Z^{2a}; preferably each Z² is independently selected from halo, cyano, oxo, or from the group comprising C₁₋₂alkyl, haloC₁₋₂alkyl, C₁₋₂alkoxy, C₁₋₂alkylthio, haloC₁₋₂alkoxy, hydroxyC₁₋₂alkyl, C₁₋₂alkoxyC₁₋₂alkyl, C₃₋₆cycloalkyloxy, and C₃₋₆cycloalkylC₁₋₂alkoxy, wherein each of said group can be unsubstituted or substituted with one or more Z^{2a};
and/or two Z² together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{2a}; preferably and/or two Z² together with the atom(s) to which they are attached can form a C₆₋₁₀aryl, or a 5-10 membered heteroaryl; wherein each of said C₆₋₁₀aryl and heteroaryl, can be unsubstituted or substituted with one or more Z^{2a}; preferably and/or two Z² together with the atom(s) to which they are attached can form a C₆₋₁₀aryl, or a 5-8 membered heteroaryl; wherein each of said C₆₋₁₀aryl and heteroaryl, can be unsubstituted or substituted with one or more Z^{2a}; preferably and/or two Z² together with the atom(s) to which they are attached can form a phenyl, or a 5-6 membered heteroaryl; wherein each of said phenyl, and heteroaryl, can be unsubstituted or substituted with one or more Z^{2a};
each Z^{2a} is independently selected from the group comprising halo, cyano, hydroxyl, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkenyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; preferably each Z^{2a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; preferably each Z²a is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, and oxo; preferably each Z^{2a} is independently selected from the group comprising halo, cyano, hydroxyl, C₁₋₆alkyl, haloC₁₋₆alkyl, C₁₋₆alkoxy, haloC₁₋₆alkoxy, hydroxyC₁₋₆alkyl, and oxo. 9. The compound according to any one of statements 1-8, wherein R³ is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; preferably R³ is selected from the group comprising hydrogen, halo, cyano, C₁₋₆alkyl, haloC₁₋₆alkyl, C₁₋₆alkoxy, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; preferably R³ is selected from the group comprising hydrogen, halo, cyano, C₁₋₆alkyl, haloC₁₋₆alkyl, C₁₋₆alkoxy, and haloC₁₋₆alkoxy; preferably R³ is selected from the group comprising hydrogen, halo, cyano, and C₁₋₆alkyl; preferably R³ is selected from hydrogen, or C₁₋₆alkyl; preferably R³ is selected from hydrogen, or C₁₋₄alkyl; preferably R³ is selected from hydrogen, or C₁₋₂alkyl; preferably R³ is selected from hydrogen, or methyl; preferably R³ is hydrogen. 10. The compound according to any one of statements 1-9, wherein R⁴ is C₆₋₁₀aryl, or 5-10 membered heteroaryl; preferably R⁴ is C₆₋₁₀aryl, or 5-8 membered heteroaryl; preferably R⁴ is phenyl, or 5-6 membered heteroaryl; preferably wherein the 5-6 membered heteroaryl is selected from the group comprising pyridyl, pyrrolyl, pyrazinyl, pyridazinyl, pyrimidinyl, thiophenyl, furanyl, thiazolyl, isothiazolyl, and 1,2,5-thiadiazolyl, phenyl, or pyridyl;
wherein each of said C₆₋₁₀aryl and 5-10 membered heteroaryl, is substituted with one or more Z⁴; preferably wherein each of said C₆₋₁₀aryl and 5-10 membered heteroaryl, is substituted with two or more Z⁴.

11. The compound according to any one of statements 1-10, wherein each Z⁴ is independently selected from halo, cyano, hydroxyl, oxo, nitro, thioxo, or from the group comprising C₁₋₆alkyl, C₂₋₆alkenyl, C₃₋₁₀cycloalkyl, C₃₋₁₀cycloalkylC₁₋₆alkyl, C₅₋₁₀cycloalkenyl, C₆₋₁₀aryl, C₆₋₁₀arylC₁₋₆alkyl, haloC₁₋₆alkyl, haloC₂₋₆alkenyl, cyanoC₁₋₆alkyl, C₁₋₆alkoxy, C₂₋₆alkenyloxy, cyanoC₁₋₆alkoxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀arylC₂₋₆alkenyl, haloC₂₋₆alkenyloxy, hydroxyC₂₋₆alkenyl, C₂₋₆alkenyloxyC₁₋₆alkyl, C₂₋₆alkenyloxyC₁₋₆alkoxy, C₂₋₆alkenyloxycarbonyl, C₂₋₆alkenylcarbonyl, aminoC₂₋₆alkenyl, mono or di(C₁₋₆alkyl)aminoC₂₋₆alkenyl, 3-10 membered saturated or partially saturated heterocyclylC₂₋₆alkenyl, 5-10 membered heteroarylC₂₋₆alkenyl, C₆₋₁₀aryloxy, C₆₋₁₀aryloxyC₁₋₆alkyl, C₆₋₁₀aryloxyC₂₋₆alkenyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₂₋₆alkenyloxycarbonylamino, C₁₋₆alkylcarbonylamino, C₂₋₆alkenylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂₋₆alkenylcarbonyloxy, and C₆₋₁₀arylcarbonyloxy; each of said group can be unsubstituted or substituted with one or more Z^{4a}; preferably each Z⁴ is independently selected from halo, cyano, hydroxyl, oxo, nitro, thioxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀aryloxy, C₆₋₁₀aryloxyC₁₋₆alkyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀-cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₁₋₆alkylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, and C₆₋₁₀arylcarbonyloxy; each of said group can be unsubstituted or substituted with one or more Z^{4a}; preferably each Z⁴ is independently selected from halo, cyano, hydroxyl, oxo, nitro, thioxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{4a}; preferably each Z⁴ is independently selected from halo, cyano, hydroxyl, oxo, thioxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy; each of said group can be unsubstituted or substituted with one or more Z^{4a}; preferably each Z⁴ is independently selected from halo, cyano, hydroxyl, oxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, each of said group can be unsubstituted or substituted with one or more Z^{4a}; preferably each Z⁴ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, haloC₁. 6alkoxy, C₁₋₆alkoxyC₁₋₆alkyl, C₃₋₁₀cycloalkyloxy, C₁₋₆alkoxycarbonyl, C₁₋₆alkylcarbonyl, each of said group can be unsubstituted or substituted with one or more Z^{4a}; preferably each Z⁴ is independently selected from halo, cyano, oxo, or from the group comprising C₁₋₄alkyl, C₃₋₆cycloalkyl, C₆₋₁₀aryl, haloC₁₋₄alkyl, cyanoC₁₋₄alkyl, C₁₋₄alkoxy, cyanoC₁₋₄alkoxy, haloC₁₋₄alkoxy, C₁₋₄alkoxyC₁₋₄alkyl, C₃₋₆cycloalkyloxy, C₁₋₄alkoxycarbonyl, C₁₋₄alkylcarbonyl, each of said group can be unsubstituted or substituted with one or more Z^{4a}; preferably each Z⁴ is independently selected from halo, cyano, oxo, or from the group comprising C₁₋₂alkyl, C₃₋₆cycloalkyl, phenyl, haloC₁₋₂alkyl, cyanoC₁₋₂alkyl, C₁₋₂alkoxy, cyanoC₁₋₂alkoxy, haloC₁₋₂alkoxy, C₁₋₂alkoxyC₁₋₂alkyl, C₃₋₆cycloalkyloxy, C₁₋₂alkoxycarbonyl, C₁₋₂alkylcarbonyl, each of said group can be unsubstituted or substituted with one or more Z^{4a};
and/or two Z⁴ together with the atom(s) to which they are attached can form an C₆₋₁₀aryl, a 5-10 membered heteroaryl, a C₃₋₁₀cycloalkyl, or a 3-10 membered saturated or partially saturated heterocyclyl, wherein each of said C₆₋₁₀aryl, heteroaryl, C₃₋₁₀cycloalkyl, and heterocyclyl can be unsubstituted or substituted with one or more Z^{4a}; preferably and/or two Z⁴ together with the atom(s) to which they are attached can form an C₆₋₁₀aryl, a 5-8 membered heteroaryl, a C₃₋₁₀cycloalkyl, or a 3-8 membered saturated heterocyclyl, wherein each of said C₆₋₁₀aryl, heterocyclyl, C₃₋₁₀cycloalkyl, and heteroaryl can be unsubstituted or substituted with one or more Z^{4a}; preferably and/or two Z⁴ together with the atom(s) to which they are attached can form an phenyl, a 5-6 membered heteroaryl, a C₃₋₆cycloalkyl, or a 5-6 membered saturated heterocyclyl, wherein each of said phenyl, heterocyclyl, cycloalkyl and heteroaryl can be unsubstituted or substituted with one or more Z^{4a};
each Z^{4a} is independently selected from the group comprising halo, cyano, hydroxyl, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyl, C₅₋₁₀cycloalkenyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo, preferably each Z^{4a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

12. The compound according to any one of statements 1-11, wherein
R¹ is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl, and A¹-X¹—; and R² is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl;
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X¹ and A¹ of R¹, can be unsubstituted or substituted with one or more Z¹;
X¹ is selected from —C(R^{1a})₂—, —CO—, —O—, or —NR^{1b}—;
each R^{1a} is independently selected from the group comprising hydrogen, halo, hydroxy, and C₁₋₆alkyl;
A¹ is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl;
each Z¹ is independently selected from halo, cyano, hydroxy, oxo, nitro, thioxo, or from the group comprising C₁₋₆alkyl, C₂₋₆alkenyl, C₃₋₁₀cycloalkyl, C₃₋₁₀cycloalkylC₁₋₆alkyl, C₅₋₁₀cycloalkenyl, C₆₋₁₀aryl, C₆₋₁₀arylC₁₋₆alkyl, haloC₁₋₆alkyl, haloC₂₋₆alkenyl, cyanoC₁₋₆alkyl, C₁₋₆alkoxy, C₂₋₆alkenyloxy, cyanoC₁₋₆alkoxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆ alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl, C₆₋₁₀arylC₂₋₆alkenyl, haloC₂₋₆ alkenyloxy, hydroxyC₂₋₆alkenyl, C₂₋₆alkenyloxyC₁₋₆alkyl, C₂₋₆alkenyloxyC₁₋₆alkoxy, C₂₋₆alkenyloxycarbonyl, C₂₋₆alkenylcarbonyl, aminoC₂₋₆alkenyl, mono or di(C₁₋₆alkyl)aminoC₂₋₆alkenyl, 3-10 membered saturated or partially saturated heterocyclylC₂₋₆alkenyl, 5-10 membered heteroarylC₂₋₆alkenyl, C₆₋₁₀aryloxy, C₆₋₁₀aryloxyC₁₋₆alkyl, C₆₋₁₀aryloxyC₂₋₆alkenyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₂₋₆alkenyloxycarbonylamino, C₁₋₆alkylcarbonylamino, C₂₋₆alkenylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂₋₆alkenylcarbonyloxy, and C₆₋₁₀arylcarbonyloxy; each of said group can be unsubstituted or substituted with one or more Z^{1a};
and/or two Z¹ together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{1a};
and/or one R^{1a} together with one Z¹ and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{1a};
R^{1b} is hydrogen or C₁₋₆alkyl, or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a};
each Z^{1a} is independently selected from the group comprising halo, cyano, hydroxyl, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyl, C₆₋₁₀ cycloalkenyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo;
or R¹ is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆-alkylthio, C₂₋₆alkenylthio, haloC₁₋₆ alkoxy, C₁₋₆alkoxyC₁₋₆alkyl, mono or di(C₁₋₆alkyl)amino, and mono or di(C₁₋₆alkyl)aminoC₁₋₆alkyl; and R² is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl, and A²-X²—;
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X² and A² of R², can be unsubstituted or substituted with one or more Z²;
X² is selected from —C(R^{2a})₂—, —CO—, —O—, or —NR^{2b}—; wherein each R^{2a} is independently selected from the group comprising hydrogen, halo, hydroxy and C₁₋₆alkyl;
A² is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl;
each Z² is independently selected from halo, cyano, hydroxy, oxo, nitro, thioxo, or from the group comprising C₁₋₆alkyl, C₂₋₆alkenyl, C₃₋₁₀cycloalkyl, C₃₋₁₀cycloalkylC₁₋₆alkyl, C₅₋₁₀cycloalkenyl, C₆₋₁₀aryl, C₆₋₁₀arylC₁₋₆alkyl, haloC₁₋₆alkyl, haloC₂₋₆alkenyl, cyanoC₁₋₆alkyl, C₁₋₆alkoxy, C₂₋₆alkenyloxy, cyanoC₁₋₆alkoxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆-alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀arylC₂₋₆alkenyl, haloC₂₋₆alkenyloxy, hydroxyC₂₋₆alkenyl, C₂₋₆alkenyloxyC₁₋₆alkyl, C₂₋₆alkenyloxyC₁₋₆alkoxy, C₂₋₆alkenyloxycarbonyl, C₂₋₆alkenylcarbonyl, aminoC₂₋₆alkenyl, mono or di(C₁₋₆alkyl)aminoC₂₋₆alkenyl, 3-10 membered saturated or partially saturated heterocyclylC₂₋₆alkenyl, 5-10 membered heteroarylC₂₋₆alkenyl, C₆₋₁₀aryloxy, C₆₋₁₀aryloxyC₁₋₆alkyl, C₆₋₁₀aryloxyC₂₋₆alkenyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₂₋₆alkenyloxycarbonylamino, C₁₋₆alkylcarbonylamino, C₂₋₆alkenylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂-alkenylcarbonyloxy, and C₆₋₁₀arylcarbonyloxy; each of said group can be unsubstituted or substituted with one or more Z²a
and/or two Z² together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{2a};
and/or one R^{2a} together with one Z² and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{2a};
R^{2b} is hydrogen or C₁₋₆alkyl, or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};
each Z^{2a} is independently selected from the group comprising halo, cyano, hydroxyl, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyl, C₅₋₁₀cycloalkenyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; R³ is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, C₁₋₆alkoxyC₁₋₆alkyl, mono or di(O₁₋₆alkyl)amino, and mono or di(C₁₋₆alkyl)aminoC₁₋₆alkyl;
R⁴ is C₆₋₁₀aryl, or 5-10 membered heteroaryl;
wherein each of said C₆₋₁₀aryl and 5-10 membered heteroaryl, is substituted with one or more Z⁴;
each Z⁴ is independently selected from halo, cyano, hydroxyl, oxo, nitro, thioxo, or from the group comprising C₁₋₆alkyl, C₂₋₆alkenyl, C₃₋₁₀cycloalkyl, C₃₋₁₀cycloalkylC₁₋₆alkyl, C₅₋₁₀cycloalkenyl, C₆₋₁₀aryl, C₆₋₁₀arylC₁₋₆alkyl, haloC₁₋₆alkyl, haloC₂₋₆alkenyl, cyanoC₁₋₆alkyl, C₁₋₆alkoxy, C₂₋₆alkenyloxy, cyanoC₁₋₆alkoxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₅₋₁₀arylC₂₋₆alkenyl, haloC₂₋₆alkenyloxy, hydroxyC₂₋₆alkenyl, C₂₋₆alkenyloxyC₁₋₆alkyl, C₂₋₆alkenyloxyC₁₋₆alkoxy, C₂₋₆alkenyloxycarbonyl, C₂₋₆alkenylcarbonyl, aminoC₂₋₆alkenyl, mono or di(C₁₋₆alkyl)aminoC₂₋₆alkenyl, 3-10 membered saturated or partially saturated heterocyclylC₂₋₆alkenyl, 5-10 membered heteroarylC₂₋₆alkenyl, C₆₋₁₀aryloxy, C₆₋₁₀aryloxyC₁₋₆alkyl, C₆₋₁₀aryloxyC₂₋₆alkenyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₂₋₆alkenyloxycarbonylamino, C₁₋₆alkylcarbonylamino, C₂₋₆alkenylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, C₂₋₆alkenylcarbonyloxy, and C₆₋₁₀arylcarbonyloxy; each of said group can be unsubstituted or substituted with one or more Z^{4a};
and/or two Z⁴ together with the atom(s) to which they are attached can form an C₆₋₁₀aryl, a 5-10 membered heteroaryl, a C₃₋₁₀cycloalkyl, or a 3-10 membered saturated or partially saturated heterocyclyl, wherein each of said C₆₋₁₀aryl, heteroaryl, C₃₋₁₀cycloalkyl, and heterocyclyl can be unsubstituted or substituted with one or more Z^{4a};
each Z^{4a} is independently selected from the group comprising halo, cyano, hydroxyl, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, hydroxyC₁₋₆alkyl, C₁₋₆alkoxyC₁₋₆alkyl, C₃₋₁₀cycloalkyl, C₅₋₁₀cycloalkenyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

13. The compound according to any one of statements 1-12, wherein
R¹ is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl, and A¹-X¹—; and R² is selected from the group comprising hydrogen, halo, cyano, C₁₋₆alkyl, haloC₁₋₆alkyl, C₁₋₆alkoxy, C₁₋₆alkylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl;
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X¹ and A¹ of R¹, can be unsubstituted or substituted with one or more Z¹;
X¹ is selected from —C(R^{1a})₂—, —CO—, —O—, or —NR^{1b}—;
each R^{1a} is independently selected from the group comprising hydrogen, halo, hydroxy, and C₁₋₆alkyl;
A¹ is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl;
each Z¹ is independently selected from halo, cyano, hydroxy, oxo, nitro, thioxo, or from the group comprising C₁₋₆alkyl, C₃₋₁₀cycloalkyl, C₃₋₁₀cycloalkylC₁₋₆alkyl, C₃₋₁₀aryl, C₃₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl, C₆₋₁₀aryloxy, C₆₋₁₀aryloxyC₁₋₆alkyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁. 6alkyl)aminosulfonyl, mono or di(C₁₋₆alkyl)aminosulfinyl, C₁₋₆alkoxycarbonylamino, C₁₋₆alkylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, and C₆₋₁₀arylcarbonyloxy; each of said group can be unsubstituted or substituted with one or more Z^{1a};
and/or two Z¹ together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{1a};
and/or one R^{1a} together with one Z¹ and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{1a};
R^{1b} is hydrogen or C₁₋₆alkyl, or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a};
each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo;
or R¹ is selected from the group comprising hydrogen, halo, cyano, C₁₋₆alkyl, haloC₁₋₆alkyl, C₁₋₆alkoxy, C₁₋₆alkylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; and R² is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl, and A²-X²—;
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X² and A² of R², can be unsubstituted or substituted with one or more Z²;
X² is selected from —C(R^{2a})₂—, —CO—, —O—, or —NR^{2b}—; wherein each R^{2a} is independently selected from the group comprising hydrogen, halo, hydroxy and C₁₋₆alkyl;
A² is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl;
each Z² is independently selected from halo, cyano, hydroxy, oxo, nitro, thioxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀aryloxy, C₆₋₁₀aryloxyC₁₋₆alkyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₁₋₆alkylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, and C₆₋₁₀arylcarbonyloxy; each of said group can be unsubstituted or substituted with one or more Z^{2a};
and/or two Z² together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{2a};
and/or one R^{2a} together with one Z² and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{2a};
R^{2b} is hydrogen or C₁₋₆alkyl, or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};
each Z²² is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo;
R³ is selected from the group comprising hydrogen, halo, cyano, C₁₋₆alkyl, haloC₁₋₆alkyl, C₁₋₆alkoxy, C₁₋₆alkylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl;
R⁴ is C₆₋₁₀aryl, or 5-10 membered heteroaryl;
wherein each of said C₆₋₁₀aryl and 5-10 membered heteroaryl, is substituted with one or more Z⁴;
each Z⁴ is independently selected from halo, cyano, hydroxyl, oxo, nitro, thioxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, 5-10 membered heteroarylC₁₋₆alkyl, C₆₋₁₀aryloxy, C₆₋₁₀aryloxyC₁₋₆alkyl, C₆₋₁₀arylthio, haloC₁₋₆alkythio, C₃₋₁₀cycloalkylthio, C₁₋₆alkylsulfinyl, C₁₋₆alkylsulfonyl, C₃₋₁₀cycloalkylsulfinyl, C₃₋₁₀cycloalkylsulfonyl, C₆₋₁₀arylsulfinyl, C₆₋₁₀arylsulfonyl, mono or di(C₁₋₆alkyl)aminosulfonyl, mono or di(C₁₋₆alkyl)aminosulfinyl, C₁₋₆alkoxycarbonylamino, C₁₋₆alkylcarbonylamino, C₆₋₁₀cycloalkylcarbonylamino, C₆₋₁₀arylcarbonylamino, C₃₋₁₀cycloalkylcarbonyl, C₆₋₁₀arylcarbonyl, mono or di(C₁₋₆alkyl)aminocarbonyl, C₁₋₆alkylcarbonyloxy, and C₆₋₁₀arylcarbonyloxy; each of said group can be unsubstituted or substituted with one or more Z^{4a};
and/or two Z⁴ together with the atom(s) to which they are attached can form an C₆₋₁₀aryl, a 5-10 membered heteroaryl, a C₃₋₁₀cycloalkyl, or a 3-10 membered saturated or partially saturated heterocyclyl, wherein each of said C₆₋₁₀aryl, heteroaryl, C₃₋₁₀cycloalkyl, and heterocyclyl can be unsubstituted or substituted with one or more Z^{4a};
each Z^{4a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

14. The compound according to any one of statements 1-5, 9-13, wherein
R¹ is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl, and A¹-X¹—;
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X¹ and A¹ of R¹, can be unsubstituted or substituted with one or more Z¹;
R² is selected from the group comprising hydrogen, halo, cyano, C₁₋₆alkyl, and haloC₁₋₆alkyl, C₁₋₆alkoxy;
X¹ is selected from —C(R^{1a})₂—, —CO—, —O—, or —NR_{1b}—; preferably X¹ is —C(R^{1a})₂—, —CO—, or —NR^{1b}—; preferably X¹ is —C(R^{1a})₂—, or —CO—; preferably X¹ is —C(R^{1a})₂—;
each R^{1a} is independently selected from the group comprising hydrogen, halo, hydroxy, and C₁₋₆alkyl;
A¹ is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl; preferably A¹ is selected from the group comprising C₆₋₁₀aryl, 5-10 membered heteroaryl, C₃₋₁₀cycloalkyl, and C₅₋₁₀cycloalkenyl; preferably A¹ is selected from the group comprising C₆₋₁₀aryl, 5-10 membered heteroaryl, and C₅₋₁₀cycloalkenyl;
each Z¹ is independently selected from halo, cyano, hydroxy, oxo, thioxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{1a};
and/or two Z¹ together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{1a};
and/or one R^{1a} together with one Z¹ and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{1a};
R^{1b} is hydrogen or C₁₋₆alkyl, or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a};
each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo;
preferably wherein heteroaryl is selected from the group comprising pyridinyl, pyrrolyl, thiophenyl, furanyl, thiazolyl, isothiazolyl, thiadiazolyl, triazol-2-yl, 1H-pyrazol-5-yl, pyrazolyl, imidazolyl, oxazolyl, isoxazolyl, triazolyl, oxadiazolyl, tetrazolyl, oxatriazolyl, thiatriazolyl, pyrimidinyl, pyrazinyl, pyridazinyl, oxazinyl, dioxinyl, thiazinyl, triazinyl, pyranyl, thiopyranyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, benzooxazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, benzo[c][1,2,5]oxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, 9H-purinyl, imidazo[1,2-a]pyridinyl, imidazo[1,2-a]pyrazinyl, imidazo[5,1-a]isoquinolinyl, imidazo[1,5-a]pyridinyl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; acridinyl, phthalazinyl, 1,4-dihydroindeno[1,2-c]-1H-pyrazolyl, 2,3-dihydro-1H-inden-1-one, 2,3-dihydro-1H-indenyl, 3,4-dihydroquinolin-2(1H)-one, 5,6-dihydroimidazo[5,1-a]isoquinolinyl, 8H-indeno[1,2-d]thiazolyl, benzo[d]oxazol-2(3H)-one, quinolin-2(1H)-one, quinazolin-4(1H)-one, quinazoline-2,4(1H,3H)-dione, benzo-[d]oxazolyl, and pyrazolo[1,5-a]pyridinyl,
preferably wherein the heterocyclyl is selected from the group comprising piperidinyl, piperazinyl, homopiperazinyl, morpholinyl, tetrahydropyranyl, tetrahydrofuranyl, pyrrolidinyl, aziridinyl, oxiranyl, thiiranyl, azetidinyl, oxetanyl, thietanyl, imidazolinyl, pyrazolidinyl imidazolidinyl, oxazolinyl, isoxazolinyl, oxazolidinyl, isoxazolidinyl, thiazolidinyl, isothiazolidinyl, succinimidyl, indolinyl, isoindolinyl, chromanyl (also known as 3,4-dihydrobenzo[b]pyranyl), 2H-pyrrolyl, pyrrolinyl (such as 1-pyrrolinyl, 2-pyrrolinyl, 3-pyrrolinyl), 4H-quinolizinyl, 2-oxopiperazinyl, pyrazolinyl (such as 2-pyrazolinyl, 3-pyrazolinyl), tetrahydro-2H-pyranyl, 2H-pyranyl, 4H-pyranyl, dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formyl-piperazinyl, morpholinyl, thiomorpholinyl, dihydrofuranyl, dihydrothienyl, tetrahydrothienyl, dihydropyrazolyl, dihydroimidazolyl, isothiazolinyl, thiazolinyl, triazolinyl, triazolidinyl, oxadiazolinyl, oxadiazolidinyl, thiadiazolinyl, thiadiazolidinyl, tetrazolinyl, tetrazolidinyl, dihydro-pyridinyl, tetrahydro-pyridinyl, 1,2,3,6-tetrahydropyridinyl, hexahydro-pyridinyl, dihydro-pyrimidinyl, tetrahydro-pyrimidinyl, 1,4,5,6-tetrahydropyrimidinyl, dihydro-pyrazinyl, tetrahydro-pyrazinyl, dihydro-pyridazinyl, tetrahydro-pyridazinyl, dihydro-triazinyl, tetrahydro-triazinyl, hexahydro-triazinyl, 1,4-diazepanyl, dihydro-indolyl, indolinyl, tetrahydro-indolyl, dihydro-indazolyl, tetrahydro-indazolyl, dihydro-isoindolyl, dihydro-benzofuranyl, tetrahydro-benzofuranyl, dihydro-benzothienyl, tetrahydro-benzothienyl, dihydro-benzimidazolyl, tetrahydro-benzimidazolyl, dihydro-benzooxazolyl, 2,3-dihydrobenzo[d]oxazolyl, tetrahydro-benzooxazolyl, dihydro-benzooxazinyl, 3,4-dihydro-2H-benzo[b][1,4]oxazinyl, tetrahydro-benzooxazinyl, benzo[1,3]dioxolyl, benzo[1,4]dioxanyl, dihydro-purinyl, tetrahydro-purinyl, dihydro-quinolinyl, 1,2,3,4-tetrahydroquinolinyl, dihydro-isoquinolinyl, 3,4-dihydroisoquinolin-(1H)-yl, tetrahydro-isoquinolinyl, 1,2,3,4-tetrahydroisoquinolinyl, dihydro-quinazolinyl, tetrahydro-quinazolinyl, dihydro-quinoxalinyl, tetrahydro-quinoxalinyl, 1,2,3,4-tetrahydroquinoxalinyl, 2,5-dihydro-1H-pyrrolyl, 4,5-dihydro-1H-imidazolyl, hexahydropyrrolo[3,4-b][1,4]oxazin-(2H)-yl, 3,4-dihydro-2H-pyrido[3,2-b][1,4]oxazinyl, (cis)-octahydrocyclopenta[c]pyrrolyl, hexahydropyrrolo[3,4-b]pyrrol-(1H)-yl, 5H-pyrrolo[3,4-b]pyridin-(7H)-yl, 5,7-dihydro-6H-pyrrolo[3,4-b]pyridinyl, tetrahydro-1H-pyrrolo[3,4-b]pyridin-(2H,7H,7aH)-yl, hexahydro-1H-pyrrolo[3,4-b]pyridin-(2H)-yl, (octahydro-6H-pyrrolo[3,4-b]pyridinyl, hexahydropyrrolo[1,2-a]pyrazin-(1H)-yl, 3,4,6,7,8,8a-hexahydro-1H-pyrrolo[1,2-a]pyrazinyl, 2,3,4,9-tetrahydro-1H-carbazolyl, 1,2,3,4-tetrahydropyrazino[1,2-a]indolyl, 2,3-dihydro-1H-pyrrolo[1,2-a]indolyl, 1,3-dihydro-2H-isoindolyl, octahydro-2H-isoindolyl, 2,5-diazabicyclo[2.2.1]heptanyl, 2-azabicyclo[2.2.1]heptenyl, 3-azabicyclo[3.1.0]hexanyl, 3,6-diazabicyclo[3.1.0]hexanyl, 5-azaspiro[2.4]heptanyl, 4,7-diazaspiro[2.5]octanyl, 2,6-diazaspiro[3.3]heptanyl, 2,5-diazaspiro[3.4]octanyl, 2,6-diazaspiro[3.4]octanyl, 2,7-diazaspiro[3.5]nonanyl, 2,7-diazaspiro[4.4]nonanyl, 2-azaspiro[4.5]decanyl, 2,8-diazaspiro[4.5]decanyl, 3,6-diazabicyclo[3.2.1]octyl, 1,4-dihydroindeno[1,2-c]pyrazolyl, dihydropyranyl, dihydropyridinyl, dihydroquinolinyl, 8H-indeno[1,2-d]thiazolyl, tetrahydroimidazo[1,2-a]pyridinyl, pyridin-2(1H)-one, and 8-azabicyclo[3.2.1]oct-2-enyl.

15. The compound according to any one of statements 1-5, 9-14, wherein
R¹ is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl, and A¹-X¹—;
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X¹ and A¹ of R¹, can be unsubstituted or substituted with one or more Z¹;
R² is selected from hydrogen, or C₁₋₆alkyl;
X¹ is —C(R^{1a})₂—, —CO—, or —NR^{1b}—; preferably X¹ is —C(R^{1a})₂—, or —CO—; preferably X¹ is —C(R^{1a})₂—;
each R^{1a} is independently selected from hydrogen, or C₁₋₆alkyl;
A¹ is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl; preferably A¹ is selected from the group comprising C₆₋₁₀aryl, 5-10 membered heteroaryl, C₃₋₁₀cycloalkyl, and C₅₋₁₀cycloalkenyl; preferably A¹ is selected from the group comprising C₆₋₁₀aryl, 5-10 membered heteroaryl, and C₅₋₁₀cycloalkenyl;
each Z¹ is independently selected from halo, cyano, oxo, thioxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{1a};
and/or two Z¹ together with the atom(s) to which they are attached can form a C₆₋₁₀aryl, a 5-10 membered heteroaryl, C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{1a};
and/or one R^{1a} together with one Z¹ and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{1a};
R^{1b} is hydrogen or C₁₋₆alkyl, or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z¹²;
each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

16. The compound according to any one of statements 1-5, 9-15, wherein
R¹ is selected from the group comprising C₆₋₁₀aryl, 5-10 membered heteroaryl, C₃₋₁₀cycloalkyl, C₅₋₁₀cycloalkenyl, and A¹-X¹—;
   wherein each of said C₆₋₁₀aryl, 5-10 membered heteroaryl, C₃₋₁₀cycloalkyl, C₅₋₁₀cycloalkenyl, X¹ and A¹ of R¹, can be unsubstituted or substituted with one or more Z¹;
R² is selected from hydrogen, or C₁₋₆alkyl; preferably R² is selected from hydrogen, or C₁₋₄alkyl; preferably R² is selected from hydrogen, or C₁₋₂alkyl; preferably R² is selected from hydrogen, or methyl, preferably R² is hydrogen;
X¹ is —C(R^{1a})₂—, or —CO—; preferably X¹ is —C(R^{1a})₂—;
each R^{1a} is independently selected from hydrogen, or C₁₋₆akyl;
A¹ is selected from the group comprising C₆₋₁₀aryl, 5-10 membered heteroaryl, C₃₋₁₀cycloalkyl, and C₅₋₁₀cycloalkenyl; preferably A¹ is selected from the group comprising C₆₋₁₀aryl, 5-10 membered heteroaryl, and C₆₋₁₀cycloalkenyl;
each Z¹ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{1a};
and/or two Z¹ together with the atom(s) to which they are attached can form a C₆₋₁₀aryl, or a 5-10 membered heteroaryl; wherein each of said C₆₋₁₀aryl and heteroaryl, can be unsubstituted or substituted with one or more Z^{1a};
each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

17. The compound according to any one of statements 1-5, 9-16, wherein
R¹ is selected from the group comprising C₆₋₁₀aryl, 5-10 membered heteroaryl, C₃₋₁₀cycloalkyl, C₅₋₁₀cycloalkenyl, and A¹-X¹—; preferably R¹ is selected from the group comprising C₆₋₁₀aryl, 5-8 membered heteroaryl, C₅₋₈cycloalkyl, C₃₋₈cycloalkenyl; and A¹-X¹—; preferably R¹ is selected from the group comprising phenyl, 5-6 membered heteroaryl, C₃₋₆cycloalkyl, C₅₋₆cycloalkenyl; and A¹-X¹—; preferably R¹ is selected from the group comprising phenyl, 5-6 membered heteroaryl, C₄₋₅cycloalkyl, cyclohexenyl; and A¹-X¹—;
   wherein each of said C₆₋₁₀aryl, 5-10 membered heteroaryl, C₃₋₁₀cycloalkyl, C₅₋₁₀cycloalkenyl, X¹ and A¹ of R¹, can be unsubstituted or substituted with one or more Z¹;
R² is selected from hydrogen, or C₁₋₆alkyl; preferably R² is selected from hydrogen, or C₁₋₄alkyl; preferably R² is selected from hydrogen, or C₁₋₂alkyl; preferably R² is selected from hydrogen, or methyl, preferably R² is hydrogen;
X¹ is —C(R^{1a})₂—; wherein each R^{1a} is independently selected from hydrogen, or C₁₋₆alkyl; preferably each R^{1a} is independently selected from hydrogen, or C₁₋₄alkyl; preferably each R^{1a} is independently selected from hydrogen, or C₁₋₂alkyl; preferably each R^{1a} is independently selected from hydrogen, or methyl; preferably X¹ is —CH₂—;
A¹ is selected from the group comprising C₆₋₁₀aryl, 5-10 membered heteroaryl, and C₅₋₁₀cycloalkenyl; preferably A¹ is selected from the group comprising C₆₋₁₀aryl, 5-10 membered heteroaryl, and C₅₋₁₀cycloalkenyl; preferably C₆₋₁₀aryl, 5-10 membered heteroaryl, and C₅₋₁₀cycloalkenyl; preferably A¹ is selected from the group comprising C₆₋₁₀aryl, 5-8 membered heteroaryl, and C₅₋₆cycloalkenyl; preferably A¹ is selected from the group comprising phenyl, 5-6 membered heteroaryl, and cyclohexenyl; preferably A¹ is selected from phenyl, or 5-6 membered heteroaryl; preferably A¹ is phenyl,
each Z¹ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{1a}; preferably each Z¹ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, wherein each of said group can be unsubstituted or substituted with one or more Z^{1a}; preferably each Z¹ is independently selected from halo, cyano, oxo, or from the group comprising C₁₋₆alkyl, C₃₋₁₀cycloalkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, and C₃₋₁₀cycloalkylC₁₋₆alkoxy, wherein each of said group can be unsubstituted or substituted with one or more Z^{1a}; preferably each Z^{1a} is independently selected from halo, cyano, oxo, or from the group comprising C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, and C₃₋₁₀cycloalkylC₁₋₆alkoxy, wherein each of said group can be unsubstituted or substituted with one or more Z^{1a};
and/or two Z¹ together with the atom(s) to which they are attached can form a C₆₋₁₀aryl, or a 5-10 membered heteroaryl; wherein each of said C₆₋₁₀aryl and heteroaryl, can be unsubstituted or substituted with one or more Z^{1a}; preferably and/or two Z¹ together with the atom(s) to which they are attached can form a C₆₋₁₀aryl, or a 5-8 membered heteroaryl; wherein each of said C₆₋₁₀aryl and heteroaryl, can be unsubstituted or substituted with one or more Z^{1a}; preferably and/or two Z¹ together with the atom(s) to which they are attached can form a phenyl, or a 5-6 membered heteroaryl; wherein each of said phenyl, and heteroaryl, can be unsubstituted or substituted with one or more Z^{1a};
each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; preferably each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, and oxo; preferably each Z^{1a} is independently selected from the group comprising halo, cyano, hydroxyl, C₁₋₆alkyl, haloC₁₋₆alkyl, C₁₋₆alkoxy, haloC₁₋₆alkoxy, hydroxyC₁₋₆alkyl, and oxo.

18. The compound according to any one of statements 1-5, 9-17, wherein
R¹ is selected from the group comprising phenyl, 5-6 membered heteroaryl, C₄₋₆cycloalkyl, C₅₋₆cycloalkenyl; and A¹-X¹—; preferably R¹ is selected from the group comprising phenyl, 5-6 membered heteroaryl, C₄₋₅cycloalkyl, cyclohexenyl; and A¹-X¹—; preferably R¹ is selected from the group comprising phenyl, 5-6 membered heteroaryl, C₄₋₆cycloalkyl, C₅₋₆cycloalkenyl; and A¹-X¹—; preferably R¹ is selected from the group comprising phenyl, 5-6 membered heteroaryl, C₄₋₅cycloalkyl, cyclohexenyl; preferably wherein the 5-6 membered heteroaryl is selected from the group comprising pyridyl, pyrrolyl, pyrazinyl, pyridazinyl, pyrimidinyl, thiophenyl, furanyl, thiazolyl, isothiazolyl, and 1,2,5-thiadiazolyl,
   wherein each of said phenyl, 5-6 membered heteroaryl, C₄₋₆cycloalkyl, C₅₋₆cycloalkenyl; X¹ and A¹ of R¹, can be unsubstituted or substituted with one or more Z¹;
R² is selected from hydrogen, or C₁₋₆alkyl; preferably R² is selected from hydrogen, or C₁₋₄alkyl; preferably R² is selected from hydrogen, or C₁₋₂alkyl; preferably R² is selected from hydrogen, or methyl, preferably R² is hydrogen;
X¹ is —C(R^{1a})₂—; wherein each R^{1a} is independently selected from hydrogen, or C₁₋₆alkyl; preferably each R^{1a} is independently selected from hydrogen, or C₁₋₄alkyl; preferably each R^{1a} is independently selected from hydrogen, or C₁₋₂alkyl; preferably each R^{1a} is independently selected from hydrogen, or methyl; preferably X¹ is —CH₂—;
A¹ is selected from the group comprising C₆₋₁₀aryl, 5-10 membered heteroaryl, and C₅₋₁₀cycloalkenyl; preferably A¹ is selected from the group comprising C₆₋₁₀aryl, 5-10 membered heteroaryl, and C₅₋₁₀cycloalkenyl; preferably C₆₋₁₀aryl, 5-10 membered heteroaryl, and C₅₋₁₀cycloalkenyl; preferably A¹ is selected from the group comprising C₆₋₁₀aryl, 5-8 membered heteroaryl, and C₅₋₆cycloalkenyl; preferably A¹ is selected from the group comprising phenyl, 5-6 membered heteroaryl, and cyclohexenyl; preferably A¹ is selected from phenyl, or 5-6 membered heteroaryl; preferably A¹ is phenyl, preferably wherein the 5-6 membered heteroaryl is selected from the group comprising pyridyl, pyrrolyl, pyrazinyl, pyridazinyl, pyrimidinyl, thiophenyl, furanyl, thiazolyl, isothiazolyl, and 1,2,5-thiadiazolyl,
each Z¹ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{1a}; preferably each Z¹ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, wherein each of said group can be unsubstituted or substituted with one or more Z^{1a}; preferably each Z¹ is independently selected from halo, cyano, oxo, or from the group comprising C₁₋₆alkyl, C₃₋₁₀cycloalkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, and C₃₋₁₀cycloalkylC₁₋₆alkoxy, wherein each of said group can be unsubstituted or substituted with one or more Z^{1a}; preferably each Z¹ is independently selected from halo, cyano, oxo, or from the group comprising C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀ocycloalkyloxy, and C₃₋₁₀cycloalkylC₁₋₆alkoxy, wherein each of said group can be unsubstituted or substituted with one or more Z^{1a};
and/or two Z¹ together with the atom(s) to which they are attached can form a C₆₋₁₀aryl, or a 5-10 membered heteroaryl; wherein each of said C₆₋₁₀aryl and heteroaryl, can be unsubstituted or substituted with one or more Z^{1a}; preferably and/or two Z¹ together with the atom(s) to which they are attached can form a C₆₋₁₀aryl, or a 5-8 membered heteroaryl; wherein each of said C₆₋₁₀aryl and heteroaryl, can be unsubstituted or substituted with one or more Z^{1a}; preferably and/or two Z¹ together with the atom(s) to which they are attached can form a phenyl, or a 5-6 membered heteroaryl; wherein each of said phenyl, and heteroaryl, can be unsubstituted or substituted with one or more Z^{1a};
each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; preferably each Z^{1a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, and oxo; preferably each Z^{1a} is independently selected from the group comprising halo, cyano, hydroxyl, C₁₋₆alkyl, haloC₁₋₆alkyl, C₁₋₆alkoxy, haloC₁₋₆alkoxy, hydroxyC₁₋₆alkyl, and oxo.

19. The compound according to any one of statements 1-2, 6-13, wherein
R¹ is selected from the group comprising hydrogen, halo, cyano, C₁₋₆alkyl, haloC₁₋₆alkyl, and C₁₋₆alkoxy;
R² is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl, and A²-X²—;
   wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X² and A² of R², can be unsubstituted or substituted with one or more Z²;
X² is selected from —C(R^{2a})₂—, —CO—, —O—, or —NR^{2b}—; preferably X² is —C(R^{2a})₂—, —CO—, or —NR^{2b}—; preferably X² is —C(R^{2a})₂—, or —CO—; preferably X² is —C(R^{2a})₂—; wherein each R^{2a} is independently selected from hydrogen, halo, hydroxy and C₁₋₆alkyl;
A² is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl; preferably A² is selected from the group comprising C₆₋₁₀aryl, 5-10 membered heteroaryl, C₃₋₁₀cycloalkyl, and C₅₋₁₀cycloalkenyl; preferably A² is selected from the group comprising C₆₋₁₀aryl, 5-10 membered heteroaryl, and C₅₋₁₀cycloalkenyl;
each Z² is independently selected from halo, cyano, hydroxy, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆-alkyl, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{2a};
and/or two Z² together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{2a};
and/or one R^{2a} together with one Z² and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{2a};
R^{2b} is hydrogen or C₁₋₆alkyl, or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};
each Z^{2a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.
preferably wherein heteroaryl is selected from the group comprising pyridinyl, pyrrolyl, thiophenyl, furanyl, thiazolyl, isothiazolyl, thiadiazolyl, triazol-2-yl, 1H-pyrazol-5-yl, pyrazolyl, imidazolyl, oxazolyl, isoxazolyl, triazolyl, oxadiazolyl, tetrazolyl, oxatriazolyl, thiatriazolyl, pyrimidinyl, pyrazinyl, pyridazinyl, oxazinyl, dioxinyl, thiazinyl, triazinyl, pyranyl, thiopyranyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, benzooxazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, benzo[c][1,2,5]oxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, 9H-purinyl, imidazo[1,2-a]pyridinyl, imidazo[1,2-a]pyrazinyl, imidazo[5,1-a]isoquinolinyl, imidazo[1,5-a]pyridinyl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; acridinyl, phthalazinyl, 1,4-dihydroindeno[1,2-c]-1H-pyrazolyl, 2,3-dihydro-1H-inden-1-one, 2,3-dihydro-1H-indenyl, 3,4-dihydroquinolin-2(1H)-one, 5,6-dihydroimidazo[5,1-a]isoquinolinyl, 8H-indeno[1,2-d]thiazolyl, benzo[d]oxazol-2(3H)-one, quinolin-2(1H)-one, quinazolin-4(1H)-one, quinazoline-2,4(1H,3H)-dione, benzo-[d]oxazolyl, and pyrazolo[1,5-a]pyridinyl,
preferably wherein heterocyclyl is selected from the group comprising piperidinyl, piperazinyl, homopiperazinyl, morpholinyl, tetrahydropyranyl, tetrahydrofuranyl, pyrrolidinyl, aziridinyl, oxiranyl, thiiranyl, azetidinyl, oxetanyl, thietanyl, imidazolinyl, pyrazolidinyl imidazolidinyl, oxazolinyl, isoxazolinyl, oxazolidinyl, isoxazolidinyl, thiazolidinyl, isothiazolidinyl, succinimidyl, indolinyl, isoindolinyl, chromanyl (also known as 3,4-dihydrobenzo[b]pyranyl), 2H-pyrrolyl, pyrrolinyl (such as 1-pyrrolinyl, 2-pyrrolinyl, 3-pyrrolinyl), 4H-quinolizinyl, 2-oxopiperazinyl, pyrazolinyl (such as 2-pyrazolinyl, 3-pyrazolinyl), tetrahydro-2H-pyranyl, 2H-pyranyl, 4H-pyranyl, dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formyl-piperazinyl, morpholinyl, thiomorpholinyl, dihydrofuranyl, dihydrothienyl, tetrahydrothienyl, dihydropyrazolyl, dihydroimidazolyl, isothiazolinyl, thiazolinyl, triazolinyl, triazolidinyl, oxadiazolinyl, oxadiazolidinyl, thiadiazolinyl, thiadiazolidinyl, tetrazolinyl, tetrazolidinyl, dihydro-pyridinyl, tetrahydro-pyridinyl, 1,2,3,6-tetrahydropyridinyl, hexahydro-pyridinyl, dihydro-pyrimidinyl, tetrahydro-pyrimidinyl, 1,4,5,6-tetrahydropyrimidinyl, dihydro-pyrazinyl, tetrahydro-pyrazinyl, dihydro-pyridazinyl, tetrahydro-pyridazinyl, dihydro-triazinyl, tetrahydro-triazinyl, hexahydro-triazinyl, 1,4-diazepanyl, dihydro-indolyl, indolinyl, tetrahydro-indolyl, dihydro-indazolyl, tetrahydro-indazolyl, dihydro-isoindolyl, dihydro-benzofuranyl, tetrahydro-benzofuranyl, dihydro-benzothienyl, tetrahydro-benzothienyl, dihydro-benzimidazolyl, tetrahydro-benzimidazolyl, dihydro-benzooxazolyl, 2,3-dihydrobenzo[d]oxazolyl, tetrahydro-benzooxazolyl, dihydro-benzooxazinyl, 3,4-dihydro-2H-benzo[b][1,4]oxazinyl, tetrahydro-benzooxazinyl, benzo[1,3]dioxolyl, benzo[1,4]dioxanyl, dihydro-purinyl, tetrahydro-purinyl, dihydro-quinolinyl, 1,2,3,4-tetrahydroquinolinyl, dihydro-isoquinolinyl, 3,4-dihydroisoquinolin-(1H)-yl, tetrahydro-isoquinolinyl, 1,2,3,4-tetrahydroisoquinolinyl, dihydro-quinazolinyl, tetrahydro-quinazolinyl, dihydro-quinoxalinyl, tetrahydro-quinoxalinyl, 1,2,3,4-tetrahydroquinoxalinyl, 2,5-dihydro-1H-pyrrolyl, 4,5-dihydro-1H-imidazolyl, hexahydropyrrolo[3,4-b][1,4]oxazin-(2H)-yl, 3,4-dihydro-2H-pyrido[3,2-b][1,4]oxazinyl, (cis)-octahydrocyclopenta[c]pyrrolyl, hexahydropyrrolo[3,4-b]pyrrol-(1H)-yl, 5H-pyrrolo[3,4-b]pyridin-(7H)-yl, 5,7-dihydro-6H-pyrrolo[3,4-b]pyridinyl, tetrahydro-1H-pyrrolo[3,4-b]pyridin-(2H,7H,7aH)-yl, hexahydro-1H-pyrrolo[3,4-b]pyridin-(2H)-yl, (octahydro-6H-pyrrolo[3,4-b]pyridinyl, hexahydropyrrolo[1,2-a]pyrazin-(1H)-yl, 3,4,6,7,8,8a-hexahydro-1H-pyrrolo[1,2-a]pyrazinyl, 2,3,4,9-tetrahydro-1H-carbazolyl, 1,2,3,4-tetrahydropyrazino[1,2-a]indolyl, 2,3-dihydro-1H-pyrrolo[1,2-a]indolyl, 1,3-dihydro-2H-isoindolyl, octahydro-2H-isoindolyl, 2,5-diazabicyclo[2.2.1]heptanyl, 2-azabicyclo[2.2.1]heptenyl, 3-azabicyclo[3.1.0]hexanyl, 3,6-diazabicyclo[3.1.0]hexanyl, 5-azaspiro[2.4]heptanyl, 4,7-diazaspiro[2.5]octanyl, 2,6-diazaspiro[3.3]heptanyl, 2,5-diazaspiro[3.4]octanyl, 2,6-diazaspiro[3.4]octanyl, 2,7-diazaspiro[3.5]nonanyl, 2,7-diazaspiro[4.4]nonanyl, 2-azaspiro[4.5]decanyl, 2,8-diazaspiro[4.5]decanyl, 3,6-diazabicyclo[3.2.1]octyl, 1,4-dihydroindeno[1,2-c]pyrazolyl, dihydropyranyl, dihydropyridinyl, dihydroquinolinyl, 8H-indeno[1,2-d]thiazolyl, tetrahydroimidazo[1,2-a]pyridinyl, pyridin-2(1H)-one, and 8-azabicyclo[3.2.1]oct-2-enyl.

20. The compound according to any one of statements 1-2, 6-13, 19 wherein
R¹ is selected from hydrogen, or C₁₋₆alkyl;
R² is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl, and A²-X²—;
   wherein each of said C₅₋₁₀aryl, 5-10 membered heteroaryl, C₃₋₁₀cycloalkyl, C₅₋₁₀cycloalkenyl, 3-10 membered saturated or partially saturated heterocyclyl, X² and A² of R², can be unsubstituted or substituted with one or more Z²;
X² is —C(R^{2a})₂—, —CO—, or —NR^{2b}—; preferably X² is —C(R^{2a})₂—, or —CO—; preferably X² is —C(R^{2a})₂—; wherein each R^{2a} is independently selected from hydrogen, hydroxyl, or C₁₋₆alkyl;
A² is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl; preferably A² is selected from the group comprising C₆₋₁₀aryl, 5-10 membered heteroaryl, C₃₋₁₀cycloalkyl, and C₅₋₁₀cycloalkenyl; preferably A² is selected from the group comprising C₆₋₁₀aryl, 5-10 membered heteroaryl, and C₅₋₁₀cycloalkenyl;
each Z² is independently selected from halo, cyano, oxo, thioxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{2a};
and/or two Z² together with the atom(s) to which they are attached can form a C₆₋₁₀aryl, a 5-10 membered heteroaryl, a C₃₋₁₀cycloalkyl, or a 3-10 membered saturated or partially saturated heterocyclyl; wherein each of said C₆₋₁₀aryl, heteroaryl, C₃₋₁₀cycloalkyl, and heterocyclyl can be unsubstituted or substituted with one or more Z^{2a};
and/or one R^{2a} together with one Z² and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{2a}; R^{2b} is hydrogen or C₁₋₆alkyl, or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};
each Z^{2a} is independently selected from the group comprising halo, cyano, hydroxyl, C₁₋₆alkyl, haloC₁₋₆ alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

21. The compound according to any one of statements 1-2, 6-13, 19-20 wherein
R¹ is selected from hydrogen, or C₁₋₆alkyl; preferably R¹ is selected from hydrogen, or C₁₋₄alkyl; preferably R¹ is selected from hydrogen, or C₁₋₂alkyl; preferably R¹ is selected from hydrogen, or methyl; preferably R¹ is hydrogen;
R² is selected from the group comprising C₆₋₁₀aryl, 5-10 membered heteroaryl, C₃₋₁₀cycloalkyl, C₅₋₁₀cycloalkenyl, and A²-X²—;
   wherein each of said C₆₋₁₀aryl, 5-10 membered heteroaryl, C₃₋₁₀cycloalkyl, C₅₋₁₀cycloalkenyl, X² and A² of R², can be unsubstituted or substituted with one or more Z²;
X² is —C(R^{2a})₂—, or —CO—; preferably X² is —C(R^{2a})₂—; wherein each R^{2a} is independently selected from hydrogen, hydroxyl, or C₁₋₆alkyl;
A² is selected from the group comprising C₆₋₁₀aryl, 5-10 membered heteroaryl, C₃₋₁₀cycloalkyl, and C₅₋₁₀cycloalkenyl; preferably A² is selected from the group comprising C₆₋₁₀aryl, 5-10 membered heteroaryl, and C₅₋₁₀cycloalkenyl;
each Z² is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆ alkoxy, C₁₋₆alkylthio, haloC₁₋₆alkoxy, hydroxyC₁₋₆-alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{2a};
and/or two Z² together with the atom(s) to which they are attached can form a C₆₋₁₀aryl, or a 5-10 membered heteroaryl; wherein each of said C₆₋₁₀aryl and heteroaryl, can be unsubstituted or substituted with one or more Z^{2a};
each Z^{2a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

22. The compound according to any one of statements 1-2, 6-13, 19-21, wherein
R¹ is selected from hydrogen, or C₁₋₆alkyl; preferably R¹ is selected from hydrogen, or C₁₋₄ alkyl; preferably R¹ is selected from hydrogen, or C₁₋₂alkyl; preferably R¹ is selected from hydrogen, or methyl; preferably R¹ is hydrogen;
R² is selected from the group comprising C₆₋₁₀aryl, 5-10 membered heteroaryl, C₃₋₁₀cycloalkyl, C₅₋₁₀cycloalkenyl, and A²-X²—;
   wherein each of said C₆₋₁₀aryl, 5-10 membered heteroaryl, C₃₋₁₀cycloalkyl, C₅₋₁₀cycloalkenyl, X² and A² of R², can be unsubstituted or substituted with one or more Z²;
X² is —C(R^{2a})₂—, or —CO—; preferably X² is —C(R^{2a})₂—; wherein each R^{2a} is independently selected from hydrogen, hydroxyl, or C₁₋₆alkyl; preferably each R^{2a} is independently selected from hydrogen, hydroxyl or C₁₋₄alkyl; preferably each R^{2a} is independently selected from hydrogen, hydroxyl or C₁₋₂alkyl; preferably each R^{2a} is independently selected from hydrogen, hydroxyl, or methyl; preferably X² is —CH₂—;
A² is selected from the group comprising C₆₋₁₀aryl, 5-10 membered heteroaryl, C₃₋₁₀cycloalkyl, and C₅₋₁₀cycloalkenyl; preferably A² is selected from the group comprising C₆₋₁₀aryl, 5-10 membered heteroaryl, C₃₋₈cycloalkyl, and C₅₋₁₀cycloalkenyl; preferably A² is selected from the group comprising C₆₋₁₀aryl, 5-8 membered heteroaryl, C₃₋₆cycloalkyl, and C₅₋₆cycloalkenyl; preferably A² is selected from the group comprising phenyl, 5-6 membered heteroaryl, C₃₋₆cycloalkyl, and C₅₋₆cycloalkenyl;
each Z² is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl; each of said group can be unsubstituted or substituted with one or more Z^{2a}; preferably each Z² is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, each of said group can be unsubstituted or substituted with one or more Z^{2a}; preferably each Z² is independently selected from halo, cyano, oxo, or from the group comprising C₁₋₆alkyl, C₃₋₁₀cycloalkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, each of said group can be unsubstituted or substituted with one or more Z^{2a}; preferably each Z² is independently selected from halo, cyano, oxo, or from the group comprising C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, each of said group can be unsubstituted or substituted with one or more Z^{2a}
each Z^{2a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; preferably each Z^{2a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, and oxo; preferably each Z^{2a} is independently selected from the group comprising halo, cyano, hydroxyl, C₁₋₆alkyl, haloC₁₋₆alkyl, C₁₋₆alkoxy, haloC₁₋₆alkoxy, hydroxyC₁₋₆alkyl, and oxo.

23. The compound according to any one of statements 1-2, 6-13, 19-22, wherein
R¹ is selected from hydrogen, or C₁₋₆alkyl; preferably R¹ is selected from hydrogen, or C₁₋₄ alkyl; preferably R¹ is selected from hydrogen, or C₁₋₂alkyl; preferably R¹ is selected from hydrogen, or methyl; preferably R¹ is hydrogen;
R² is selected from the group comprising C₆₋₁₀aryl, 5-8 membered heteroaryl, C₃₋₈cycloalkyl, C₅₋₆cycloalkenyl, and A²-X²—; preferably R² is selected from the group comprising phenyl, 5-6 membered heteroaryl, C₃₋₆cycloalkyl, C₅₋₆cycloalkenyl, and A²-X²—; preferably R² is selected from the group comprising phenyl, 5-6 membered heteroaryl, C₅₋₆cycloalkyl, C₅₋₆cycloalkenyl, and A²-X²—; preferably R² is selected from the group comprising phenyl, 5-6 membered heteroaryl, cyclopentenyl, and A²-X²—; preferably R² is selected from phenyl, or A²-X²—; preferably R² is A²-X²—; preferably wherein the 5-6 membered heteroaryl is selected from the group comprising pyridyl, pyrrolyl, pyrazinyl, pyridazinyl, pyrimidinyl, thiophenyl, furanyl, thiazolyl, isothiazolyl, and 1,2,5-thiadiazolyl,
   wherein each of said C₆₋₁₀aryl, 5-10 membered heteroaryl, C₃₋₁₀cycloalkyl, C₅₋₁₀cycloalkenyl, X² and A² of R², can be unsubstituted or substituted with one or more Z²;
X² is —C(R^{2a})₂—; wherein each R^{2a} is independently selected from hydrogen, hydroxyl, or C₁₋₆alkyl; preferably each R^{2a} is independently selected from hydrogen, hydroxyl or C₁₋₄alkyl; preferably each R^{2a} is independently selected from hydrogen, hydroxyl or C₁₋₂alkyl; preferably each R^{2a} is independently selected from hydrogen, hydroxyl, or methyl; preferably X² is —CH₂—;
A² is selected from the group comprising C₆₋₁₀aryl, 5-10 membered heteroaryl, C₃₋₁₀cycloalkyl, and C₅₋₁₀cycloalkenyl; preferably A² is selected from the group comprising C₆₋₁₀aryl, 5-10 membered heteroaryl, C₃₋₆cycloalkyl, and C₅₋₁₀cycloalkenyl; preferably A² is selected from the group comprising C₆₋₁₀aryl, 5-8 membered heteroaryl, C₃₋₆cycloalkyl, and C₅₋₆cycloalkenyl; preferably A² is selected from the group comprising phenyl, 5-6 membered heteroaryl, C₃₋₆cycloalkyl, and C₅₋₆cycloalkenyl;
each Z² is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆alkylthio, haloC₁₋₆ alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl; each of said group can be unsubstituted or substituted with one or more Z^{2a}; preferably each Z² is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, C₁₋₆ alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, each of said group can be unsubstituted or substituted with one or more Z^{2a}; preferably each Z² is independently selected from halo, cyano, oxo, or from the group comprising C₁₋₆alkyl, C₃₋₁₀cycloalkyl, haloC₁₋₆alkyl, cyanoC₁₋₆alkyl, C₁₋₆ alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, each of said group can be unsubstituted or substituted with one or more Z^{2a}; preferably each Z² is independently selected from halo, cyano, oxo, or from the group comprising C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, each of said group can be unsubstituted or substituted with one or more Z^{2a}
each Z^{2a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo; preferably each Z^{2a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, and oxo; preferably each Z^{2a} is independently selected from the group comprising halo, cyano, hydroxyl, C₁₋₆alkyl, haloC₁₋₆alkyl, C₁₋₆alkoxy, haloC₁₋₆alkoxy, hydroxyC₁₋₆alkyl, and oxo.

24. The compound according to any one of statements 1-23, wherein
R⁴ is C₆₋₁₀aryl, or 5-10 membered heteroaryl; preferably R⁴ is C₆₋₁₀aryl, or 5-8 membered heteroaryl; preferably R⁴ is phenyl, or 5-6 membered heteroaryl;
wherein each of said C₆₋₁₀aryl and 5-10 membered heteroaryl, is substituted with one or more Z⁴; preferably wherein each of said C₆₋₁₀aryl and 5-10 membered heteroaryl, is substituted with two or more Z⁴;
each Z⁴ is independently selected from halo, cyano, hydroxyl, oxo, nitro, thioxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, mono or di(C₁₋₆alkyl)amino, mono or di(C₁₋₆alkyl)aminoC₁₋₆alkyl, mono or di(C₁₋₆alkyl)aminocarbonyl, aminoC₁₋₆alkyl, amino, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{4a}; preferably each Z⁴ is independently selected from halo, cyano, hydroxyl, oxo, nitro, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{4a}; preferably each Z⁴ is independently selected from halo, cyano, hydroxyl, oxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy; each of said group can be unsubstituted or substituted with one or more Z^{4a}; preferably each Z⁴ is independently selected from halo, cyano, hydroxyl, oxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, each of said group can be unsubstituted or substituted with one or more Z^{4a}; preferably each Z⁴ is independently selected from halo, cyano, oxo, or from the group comprising C₁₋₆alkyl, C₃₋₁₀cycloalkyl, C₆₋₁₀aryl, haloC₁₋₆ alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₁₋₆alkoxycarbonyl, C₁₋₆alkylcarbonyl, each of said group can be unsubstituted or substituted with one or more Z^{4a};
and/or two Z⁴ together with the atom(s) to which they are attached can form an C₆₋₁₀aryl, a 5-10 membered heteroaryl, a C₃₋₁₀cycloalkyl, or a 3-10 membered saturated or partially saturated heterocyclyl, wherein each of said C₆₋₁₀aryl, heteroaryl, C₃₋₁₀cycloalkyl, and heterocyclyl can be unsubstituted or substituted with one or more Z^{4a}; preferably and/or two Z⁴ together with the atom(s) to which they are attached can form an C₆₋₁₀aryl, a 5-8 membered heteroaryl, a C₃₋₁₀cycloalkyl, or a 3-8 membered saturated heterocyclyl, wherein each of said C₆₋₁₀aryl, heterocyclyl, C₃₋₁₀cycloalkyl, and heteroaryl can be unsubstituted or substituted with one or more Z^{4a}; preferably and/or two Z⁴ together with the atom(s) to which they are attached can form an phenyl, a 5-6 membered heteroaryl, a C₃₋₆cycloalkyl, or a 5-6 membered saturated heterocyclyl, wherein each of said phenyl, heterocyclyl, cycloalkyl and heteroaryl can be unsubstituted or substituted with one or more Z^{4a};
each Z^{4a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo;
preferably wherein heteroaryl is selected from the group comprising pyridinyl, pyrrolyl, thiophenyl, furanyl, thiazolyl, isothiazolyl, thiadiazolyl, triazol-2-yl, 1H-pyrazol-5-yl, pyrazolyl, imidazolyl, oxazolyl, isoxazolyl, triazolyl, oxadiazolyl, tetrazolyl, oxatriazolyl, thiatriazolyl, pyrimidinyl, pyrazinyl, pyridazinyl, oxazinyl, dioxinyl, thiazinyl, triazinyl, pyranyl, thiopyranyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, benzooxazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, benzo[c][1,2,5]oxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, 9H-purinyl, imidazo[1,2-a]pyridinyl, imidazo[1,2-a]pyrazinyl, imidazo[5,1-a]isoquinolinyl, imidazo[1,5-a]pyridinyl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; acridinyl, phthalazinyl, 1,4-dihydroindeno[1,2-c]-1H-pyrazolyl, 2,3-dihydro-1H-inden-1-one, 2,3-dihydro-1H-indenyl, 3,4-dihydroquinolin-2(1H)-one, 5,6-dihydroimidazo[5,1-a]isoquinolinyl, 8H-indeno[1,2-d]thiazolyl, benzo[d]oxazol-2(3H)-one, quinolin-2(1H)-one, quinazolin-4(1H)-one, quinazoline-2,4(1H,3H)-dione, benzo-[d]oxazolyl, and pyrazolo[1,5-a]pyridinyl,
preferably wherein heterocyclyl is selected from the group comprising piperidinyl, piperazinyl, homopiperazinyl, morpholinyl, tetrahydropyranyl, tetrahydrofuranyl, pyrrolidinyl, aziridinyl, oxiranyl, thiiranyl, azetidinyl, oxetanyl, thietanyl, imidazolinyl, pyrazolidinyl imidazolidinyl, oxazolinyl, isoxazolinyl, oxazolidinyl, isoxazolidinyl, thiazolidinyl, isothiazolidinyl, succinimidyl, indolinyl, isoindolinyl, chromanyl (also known as 3,4-dihydrobenzo[b]pyranyl), 2H-pyrrolyl, pyrrolinyl (such as 1-pyrrolinyl, 2-pyrrolinyl, 3-pyrrolinyl), 4H-quinolizinyl, 2-oxopiperazinyl, pyrazolinyl (such as 2-pyrazolinyl, 3-pyrazolinyl), tetrahydro-2H-pyranyl, 2H-pyranyl, 4H-pyranyl, dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formyl-piperazinyl, morpholinyl, thiomorpholinyl, dihydrofuranyl, dihydrothienyl, tetrahydrothienyl, dihydropyrazolyl, dihydroimidazolyl, isothiazolinyl, thiazolinyl, triazolinyl, triazolidinyl, oxadiazolinyl, oxadiazolidinyl, thiadiazolinyl, thiadiazolidinyl, tetrazolinyl, tetrazolidinyl, dihydro-pyridinyl, tetrahydro-pyridinyl, 1,2,3,6-tetrahydropyridinyl, hexahydro-pyridinyl, dihydro-pyrimidinyl, tetrahydro-pyrimidinyl, 1,4,5,6-tetrahydropyrimidinyl, dihydro-pyrazinyl, tetrahydro-pyrazinyl, dihydro-pyridazinyl, tetrahydro-pyridazinyl, dihydro-triazinyl, tetrahydro-triazinyl, hexahydro-triazinyl, 1,4-diazepanyl, dihydro-indolyl, indolinyl, tetrahydro-indolyl, dihydro-indazolyl, tetrahydro-indazolyl, dihydro-isoindolyl, dihydro-benzofuranyl, tetrahydro-benzofuranyl, dihydro-benzothienyl, tetrahydro-benzothienyl, dihydro-benzimidazolyl, tetrahydro-benzimidazolyl, dihydro-benzooxazolyl, 2,3-dihydrobenzo[d]oxazolyl, tetrahydro-benzooxazolyl, dihydro-benzooxazinyl, 3,4-dihydro-2H-benzo[b][1,4]oxazinyl, tetrahydro-benzooxazinyl, benzo[1,3]dioxolyl, benzo[1,4]dioxanyl, dihydro-purinyl, tetrahydro-purinyl, dihydro-quinolinyl, 1,2,3,4-tetrahydroquinolinyl, dihydro-isoquinolinyl, 3,4-dihydroisoquinolin-(1H)-yl, tetrahydro-isoquinolinyl, 1,2,3,4-tetrahydroisoquinolinyl, dihydro-quinazolinyl, tetrahydro-quinazolinyl, dihydro-quinoxalinyl, tetrahydro-quinoxalinyl, 1,2,3,4-tetrahydroquinoxalinyl, 2,5-dihydro-1H-pyrrolyl, 4,5-dihydro-1H-imidazolyl, hexahydropyrrolo[3,4-b][1,4]oxazin-(2H)-yl, 3,4-dihydro-2H-pyrido[3,2-b][1,4]oxazinyl, (cis)-octahydrocyclopenta[c]pyrrolyl, hexahydropyrrolo[3,4-b]pyrrol-(1H)-yl, 5H-pyrrolo[3,4-b]pyridin-(7H)-yl, 5,7-dihydro-6H-pyrrolo[3,4-b]pyridinyl, tetrahydro-1H-pyrrolo[3,4-b]pyridin-(2H,7H,7aH)-yl, hexahydro-1H-pyrrolo[3,4-b]pyridin-(2H)-yl, (octahydro-6H-pyrrolo[3,4-b]pyridinyl, hexahydropyrrolo[1,2-a]pyrazin-(1H)-yl, 3,4,6,7,8,8a-hexahydro-1H-pyrrolo[1,2-a]pyrazinyl, 2,3,4,9-tetrahydro-1H-carbazolyl, 1,2,3,4-tetrahydropyrazino[1,2-a]indolyl, 2,3-dihydro-1H-pyrrolo[1,2-a]indolyl, 1,3-dihydro-2H-isoindolyl, octahydro-2H-isoindolyl, 2,5-diazabicyclo[2.2.1]heptanyl, 2-azabicyclo[2.2.1]heptenyl, 3-azabicyclo[3.1.0]hexanyl, 3,6-diazabicyclo[3.1.0]hexanyl, 5-azaspiro[2.4]heptanyl, 4,7-diazaspiro[2.5]octanyl, 2,6-diazaspiro[3.3]heptanyl, 2,5-diazaspiro[3.4]octanyl, 2,6-diazaspiro[3.4]octanyl, 2,7-diazaspiro[3.5]nonanyl, 2,7-diazaspiro[4.4]nonanyl, 2-azaspiro[4.5]decanyl, 2,8-diazaspiro[4.5]decanyl, 3,6-diazabicyclo[3.2.1]octyl, 1,4-dihydroindeno[1,2-c]pyrazolyl, dihydropyranyl, dihydropyridinyl, dihydroquinolinyl, 8H-indeno[1,2-d]thiazolyl, tetrahydroimidazo[1,2-a]pyridinyl, pyridin-2(1H)-one, and 8-azabicyclo[3.2.1]oct-2-enyl.

25. The compound according to any one of statements 1-24, wherein
R⁴ is C₆₋₁₀aryl, or 5-8 membered heteroaryl; preferably R⁴ is phenyl, or 5-6 membered heteroaryl;
wherein each of said C₆₋₁₀aryl and 5-10 membered heteroaryl, is substituted with one or more Z⁴; preferably wherein each of said C₆₋₁₀aryl and 5-10 membered heteroaryl, is substituted with two or more Z⁴;
each Z⁴ is independently selected from halo, cyano, hydroxyl, oxo, nitro, thioxo, or from the group comprising C₁₋₆ alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy, 3-10 membered saturated or partially saturated heterocyclyl, 5-10 membered heteroaryl, 3-10 membered saturated or partially saturated heterocyclylC₁₋₆alkyl, and 5-10 membered heteroarylC₁₋₆alkyl; each of said group can be unsubstituted or substituted with one or more Z^{4a}; preferably each Z⁴ is independently selected from halo, cyano, hydroxyl, oxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy; each of said group can be unsubstituted or substituted with one or more Z^{4a}; preferably each Z⁴ is independently selected from halo, cyano, hydroxyl, oxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, each of said group can be unsubstituted or substituted with one or more Z^{4a}; preferably each Z⁴ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₁₋₆alkoxycarbonyl, C₁₋₆alkylcarbonyl, each of said group can be unsubstituted or substituted with one or more Z^{4a};
and/or two Z⁴ together with the atom(s) to which they are attached can form an C₆₋₁₀aryl, a 5-8 membered heteroaryl, a C₃₋₁₀cycloalkyl, or a 3-8 membered saturated heterocyclyl, wherein each of said C₆₋₁₀aryl, heterocyclyl, C₃₋₁₀cycloalkyl, and heteroaryl can be unsubstituted or substituted with one or more Z^{4a}; preferably and/or two Z⁴ together with the atom(s) to which they are attached can form an phenyl, a 5-6 membered heteroaryl, C₃₋₆cycloalkyl, or a 5-6 membered saturated heterocyclyl, wherein each of said phenyl, heterocyclyl, cycloalkyl, and heteroaryl can be unsubstituted or substituted with one or more Z^{4a};
each Z^{4a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

26. The compound according to any one of statements 1-25, wherein
R⁴ is phenyl, or 5-6 membered heteroaryl;
wherein each of said phenyl, and 5-6 membered heteroaryl, is substituted with one or more Z⁴, preferably two or more Z⁴;
each Z⁴ is independently selected from halo, cyano, hydroxyl, oxo, thioxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy; each of said group can be unsubstituted or substituted with one or more Z^{4a}; preferably each Z⁴ is independently selected from halo, cyano, hydroxyl, oxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, each of said group can be unsubstituted or substituted with one or more Z^{4a}; preferably each Z⁴ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₁₋₆alkoxycarbonyl, C₁₋₆alkylcarbonyl, each of said group can be unsubstituted or substituted with one or more Z^{4a};
and/or two Z⁴ together with the atom(s) to which they are attached can form a phenyl, a 5-6 membered heteroaryl, C₃₋₆cycloalkyl, or a 5-6 membered saturated heterocyclyl, wherein each of said phenyl, heterocyclyl, cycloalkyl, and heteroaryl can be unsubstituted or substituted with one or more Z^{4a};
each Z^{4a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

27. The compound according to any one of statements 1-26, wherein
R⁴ is phenyl, or 5-6 membered heteroaryl; preferably wherein the 5-6 membered heteroaryl is selected from the group comprising pyridyl, pyrrolyl, pyrazinyl, pyridazinyl, pyrimidinyl, thiophenyl, furanyl, thiazolyl, isothiazolyl, and 1,2,5-thiadiazolyl phenyl, or pyridyl;
wherein each of said phenyl, and 5-6 membered heteroaryl, is substituted with one or more Z⁴, preferably two or more Z⁴;
each Z⁴ is independently selected from halo, cyano, hydroxyl, oxo, thioxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, C₁₋₆alkylthio, haloC₁₋₆alkoxy, hydroxyC₁₋₆alkyl, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, carboxyl, C₁₋₆alkoxycarbonyl, C₁₋₆alkylcarbonyl, C₆₋₁₀arylC₁₋₆alkoxy; each of said group can be unsubstituted or substituted with one or more Z^{4a}; preferably each Z⁴ is independently selected from halo, cyano, hydroxyl, oxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆ alkoxy, C₁₋₆alkoxycarbonyl, C₁₋₆alkylcarbonyl, each of said group can be unsubstituted or substituted with one or more Z^{4a}; preferably each Z⁴ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆ alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₁₋₆alkoxycarbonyl, C₁₋₆alkylcarbonyl, each of said group can be unsubstituted or substituted with one or more Z^{4a};
and/or two Z⁴ together with the atom(s) to which they are attached can form a phenyl, a 5-6 membered heteroaryl, or a 5-6 membered saturated heterocyclyl, wherein each of said phenyl, heterocyclyl and heteroaryl can be unsubstituted or substituted with one or more Z^{4a};
each Z^{4a} is independently selected from the group comprising halo, cyano, hydroxyl, C₁₋₆alkyl, haloC₁₋₆alkyl, C₁₋₆alkoxy, C₁₋₆alkylthio, haloC₁₋₆alkoxy, hydroxyC₁₋₆ alkyl, C₁₋₆alkoxyC₁₋₆alkyl, C₃₋₁₀cycloalkyl, C₃₋₁₀cycloalkyloxy, C₆₋₁₀aryl, C₆₋₁₀arylC₁₋₆alkyl, amino, mono or di(C₁₋₆alkyl)amino, mono or di(C₁₋₆alkyl)aminoC₁₋₆alkyl, and oxo.

28. The compound according to any one of statements 1-27, wherein
R⁴ is phenyl, or 5-6 membered heteroaryl; preferably wherein the 5-6 membered heteroaryl is selected from the group comprising pyridyl, pyrazinyl, pyridazinyl, pyrimidinyl, pyrrolyl, thiophenyl, furanyl, thiazolyl, isothiazolyl, and 1,2,5-thiadiazolyl, more preferably phenyl, or pyridyl;
wherein each of said phenyl, and 5-6 membered heteroaryl, is substituted with two or more Z⁴;
each Z⁴ is independently selected from halo, cyano, hydroxyl, oxo, or from the group comprising C₁₋₆alkyl, C₃₋₁₀cycloalkyl, C₃₋₁₀cycloalkylC₁₋₆alkyl, C₆₋₁₀aryl, C₆₋₁₀arylC₁₋₆alkyl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₃₋₁₀cycloalkylC₁₋₆alkoxy, C₁₋₆alkoxyC₁₋₆alkoxy, C₁₋₆alkoxycarbonyl, C₁₋₆alkylcarbonyl, each of said group can be unsubstituted or substituted with one or more Z^{4a}; preferably each Z⁴ is independently selected from halo, cyano, oxo, or from the group comprising C₁₋₆alkyl, C₃₋₁₀cycloalkyl, C₆₋₁₀aryl, haloC₁₋₆alkyl, cyanoC₁₋₆alkyl, C₁₋₆alkoxy, cyanoC₁₋₆alkoxy, haloC₁₋₆alkoxy, C₁₋₆alkoxyC₁₋₆alkyl, C₃₋₁₀cycloalkyloxy, C₁₋₆alkoxycarbonyl, C₁₋₆alkylcarbonyl, each of said group can be unsubstituted or substituted with one or more Z⁴a;
and/or two Z⁴ together with the atom(s) to which they are attached can form a phenyl, a 5-6 membered heteroaryl (such as 1,2,5-thiadiazolyl), or a 5-6 membered saturated heterocyclyl (such as 1,3-dioxolanyl), wherein each of said phenyl, heterocyclyl and heteroaryl can be unsubstituted or substituted with one or more Z^{4a};
each Z^{4a} is independently selected from the group comprising halo, cyano, hydroxyl, C₁₋₆alkyl, haloC₁₋₆alkyl, C₁₋₆alkoxy, haloC₁₋₆alkoxy, hydroxyC₁₋₆alkyl, C₁₋₆alkoxyC₁₋₆alkyl, and oxo.

29. The compound according to any one of statements 1-28, having structural formula (II)

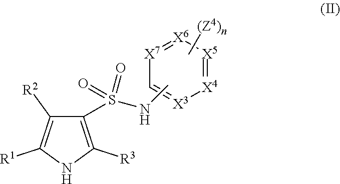

wherein each of X³, X⁴, X⁵, X⁶, and X⁷ is independently selected from CH, or N; provided that no more three X³, X⁴, X⁵, X⁶, and X⁷ are N; n is an integer selected from 1, 2, 3, or 4; and R¹, R², R³ and Z⁴ have the same meaning as in any one of statements 1-28.

30. The compound according to any one of statements 1-29, having structural formula (III) or (IV)

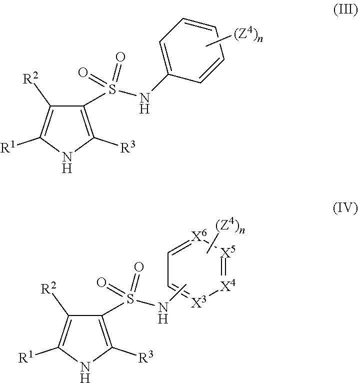

wherein each of X³, X⁴, X⁵, and X⁶, is independently selected from CH, or N; and one or two of X³, X⁴, X⁵, X⁶ is N, n is an integer selected from 1, 2, 3, or 4;
and R¹, R², R³ and Z⁴ have the same meaning as in any one of statements 1-28.

31. The compound according to any one of statements 1-30 having structural formula (V), (VI), (VII), (VIII), (Va), (VIa), (VIIa), or (VIIIa),

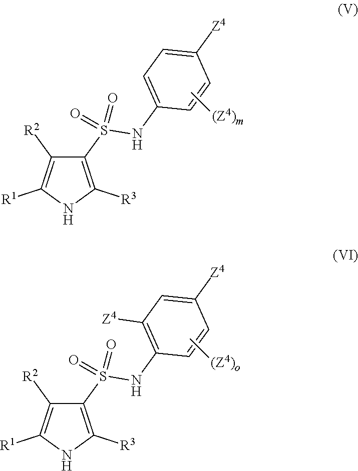

wherein each of X³, X⁶, is independently selected from CH, or N; and at least one of X³, X⁶ is N; preferably only one of X³, X⁶ is N;
wherein m is an integer selected from 0, 1, 2, or 3;
o is an integer selected from 0, 1, or 2;
p is an integer selected from 0, or 1;
and R¹, R², R³ and Z⁴ have the same meaning as in any one of statements 1-28.

32. The compound according to any one of statements 1-28, having structural formula (IX), (X), or (XI),

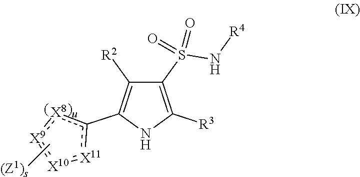

wherein each of X⁸, X⁹, X¹⁰, X¹¹, and X¹² is independently selected from CH, N, O, or S; u is an integer selected from 0, 1, 2 or 3; s is an integer selected from 0, 1, 2, 3, or 4; [symbol] is an optional double bond,
and R⁴, R¹, R², R³ and Z¹ have the same meaning as in any one of statements 1-28.

33. The compound according to any one of statements 1-28, having structural formula (XII), (XIII), (XIV), (XV), (XVI), (XVII), (XVIII), (XIX), or (XX)

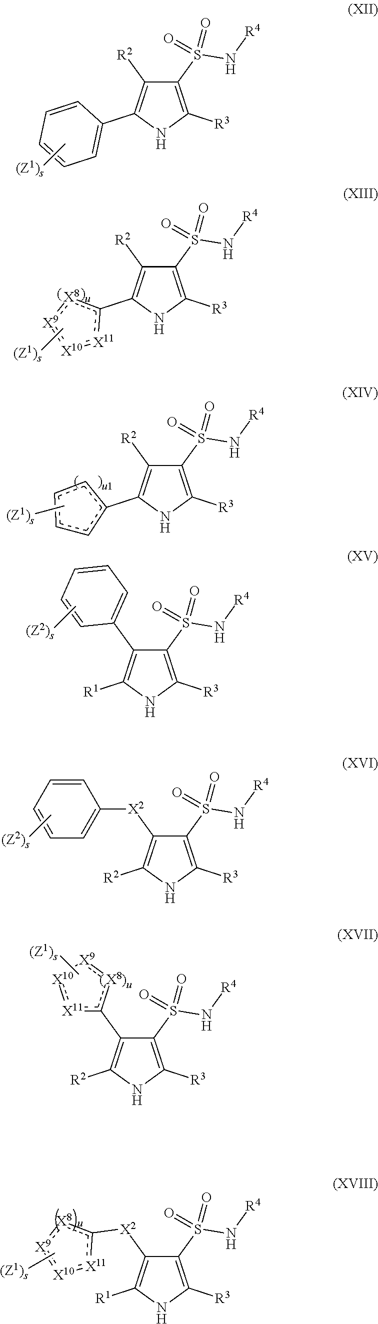

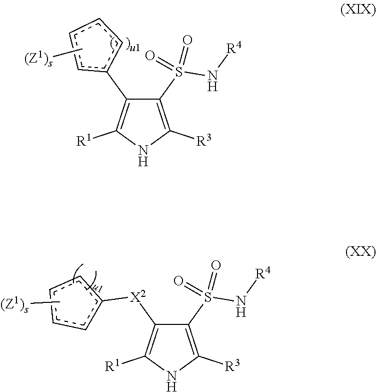

wherein each of X⁸, X⁹, X¹⁰, and X¹¹, is independently selected from CH, N, O, or S; and at least one of X⁸, X⁹, X¹⁰, and X¹¹ is selected from N, O, and S; u1 is an integer selected from 0, 1, 2, or 3; u is an integer selected from 1 or 2; s is an integer selected from 0, 1, 2, 3, 4; [symbol] is an optional double bond,
and R¹, R², R³, R⁴, and Z¹ have the same meaning as in any one of statements 1-28.

34. The compound according to any one of statements 1-28, having structural formula (II1), (II2), or (II3),

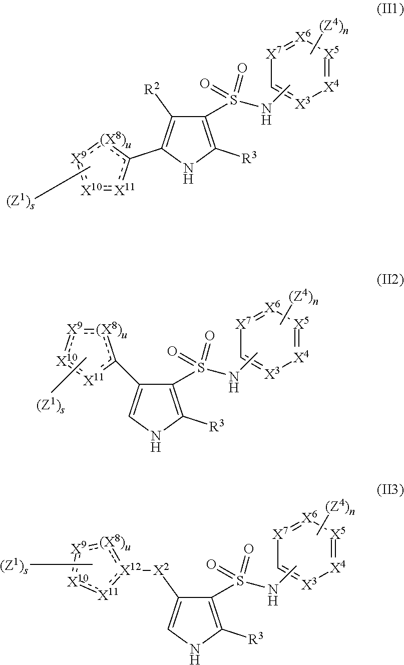

wherein each of X³, X⁴, X⁵, X⁶, and X⁷ is independently selected from CH, or N; provided that no more three X³, X⁴, X⁵, X⁶, and X⁷ are N; n is an integer selected from 1, 2, 3, or 4;
wherein each of X⁸, X⁹, X¹⁰, X¹¹, and X¹² is independently selected from CH, N, O, or S; u is an integer selected from 0, 1, 2, or 3; s is an integer selected from 0, 1, 2, 3, 4; [symbol] is an optional double bond,
and R¹, R², R³, R⁴, X², Z¹ and Z⁴ have the same meaning as in any one of statements 1-28.

35. The compound according to any one of statements 1-28, having structural formula (III1), (III2), (III3), (III4), (III5), (III6), (III7), (III8), (III9), (IV1), (IV2), (IV3), (IV4), (IV5), (IV6), (IV7), (IV8), or (IV9),

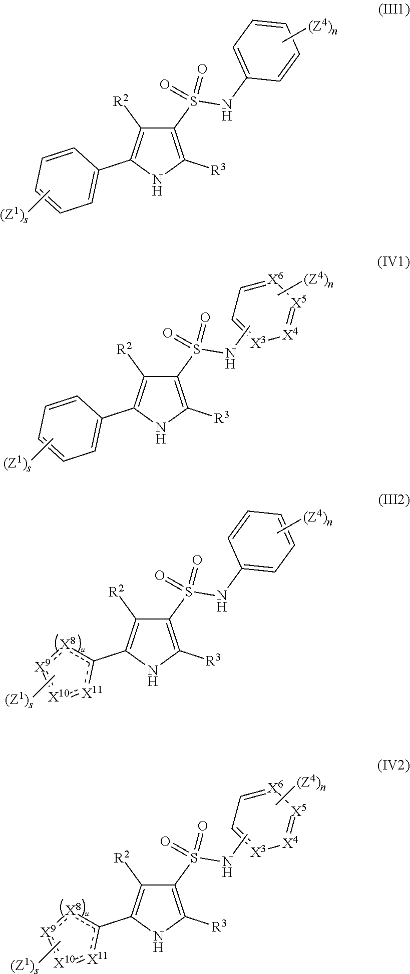

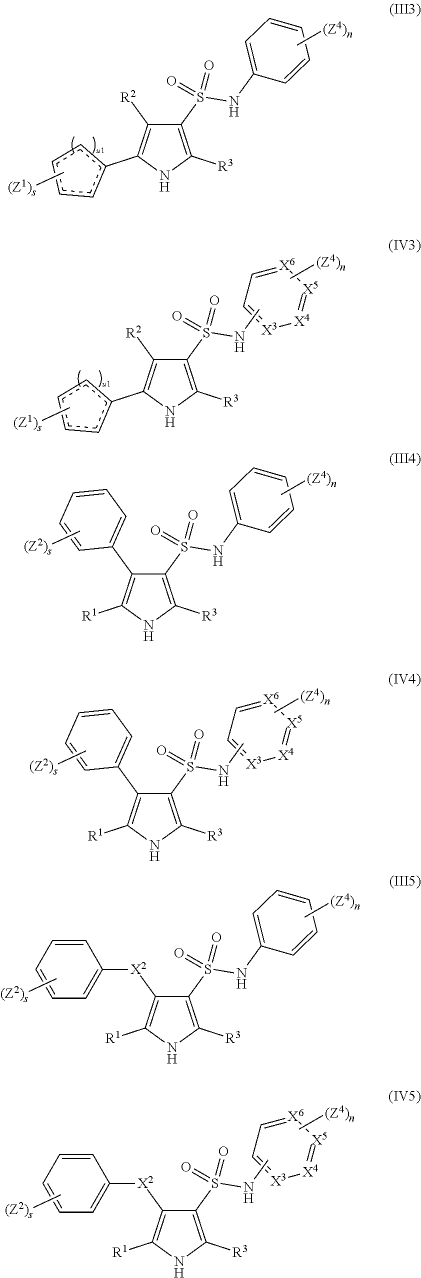

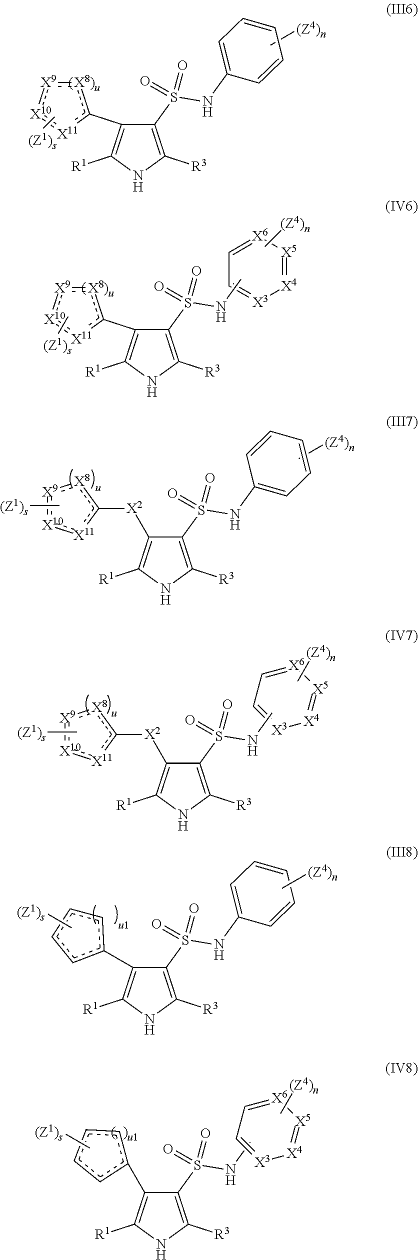

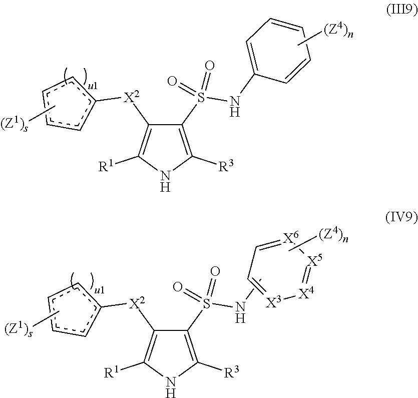

wherein each of X⁸, X⁹, X¹⁰, and X¹¹, is independently selected from CH, N, O, or S; and at least one of X⁸, X⁹, X¹⁰, and X¹¹ is selected from N, O, and S; u1 is an integer selected from 0, 1, 2, or 3; u is an integer selected from 1 or 2; n is an integer selected from 1, 2, 3, or 4; s is an integer selected from 0, 1, 2, 3, 4; [symbol] is an optional double bond,
wherein each of X³, X⁴, X⁵, and X⁶, is independently selected from CH, or N; and one or two of X³, X⁴, X⁵, and X⁶ is N, n is an integer selected from 1, 2, 3, or 4; s is an integer selected from 0, 1, 2, 3, 4;
and R¹, R², R³, R⁴, Z¹ and Z⁴ have the same meaning as in any one of statements 1-28.

36. The compound according to any one of statements 1-28, having structural formula (V1), (VI1), (VII1), (VIII1), (V2), (VI2), (VII2), (VIII2), (V3), (VI3), (VII3), (VIII3), (V4), (VI4), (VII4), (VIII4), (V5), (VI5), (VII5), (VIII5), (V6), (VI6), (VII6), (VIII6), (V7), (VI7), (VII7), (VIII7), (V8), (VI8), (VII8), (VIII8), (V9), (VI9), (VII9), (VIII9), (Va1), (VIa1), (VIIa1), (VIIIa1), (Va2), (VIa2), (VIIa2), (VIIIa2), (Va3), (VIa3), (VIIa3), (VIIIa3), (Va4), (VIa4), (VIIa4), (VIIIa4), (Va5), (VIa5), (VIIa5), (VIIIa5), (Va6), (VIa6), (VIIa6), (VIIIa6), (Va7), (VIa7), (VIIa7), (VIIIa7), (Va8), (VIa8), (VIIa8), (VIIIa8), (Va9), (VIa9), (VIIa9), or (VIIIa9),

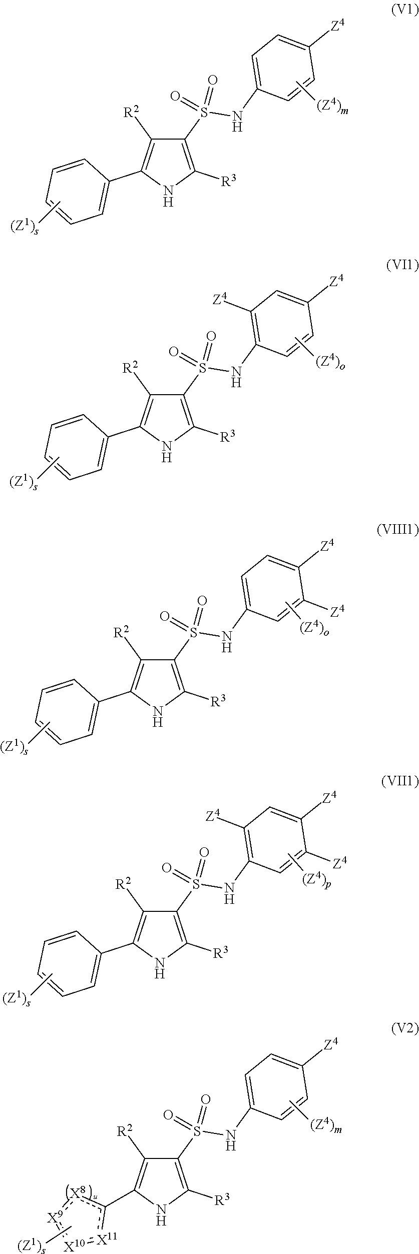

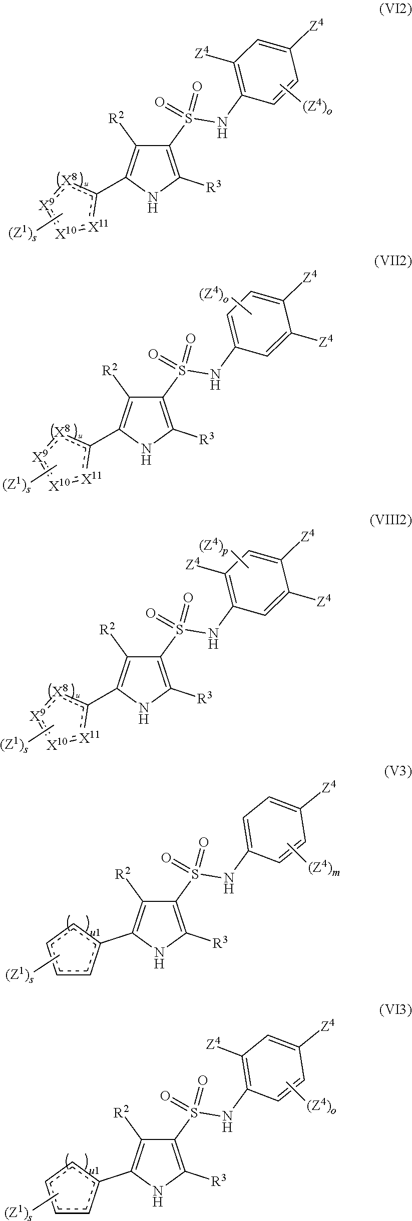

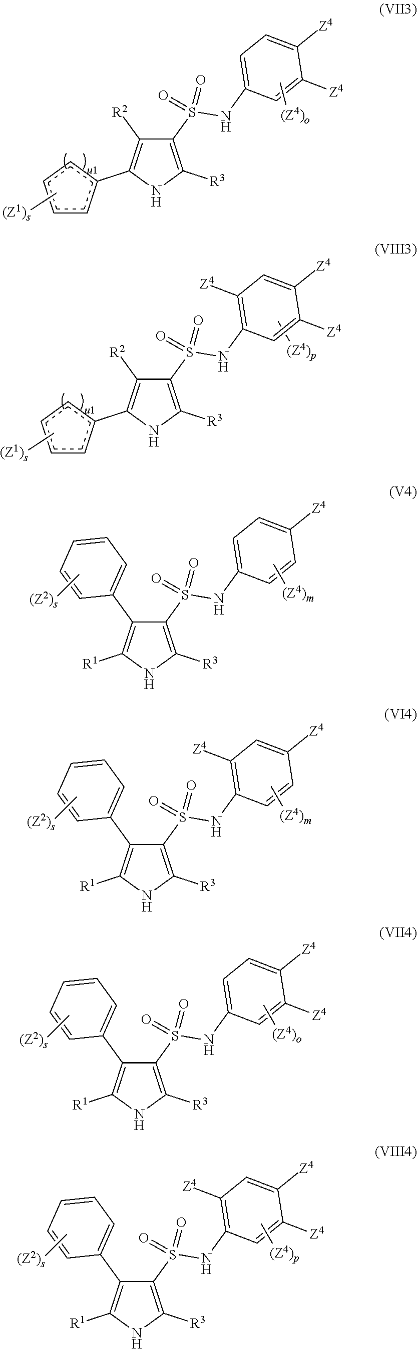

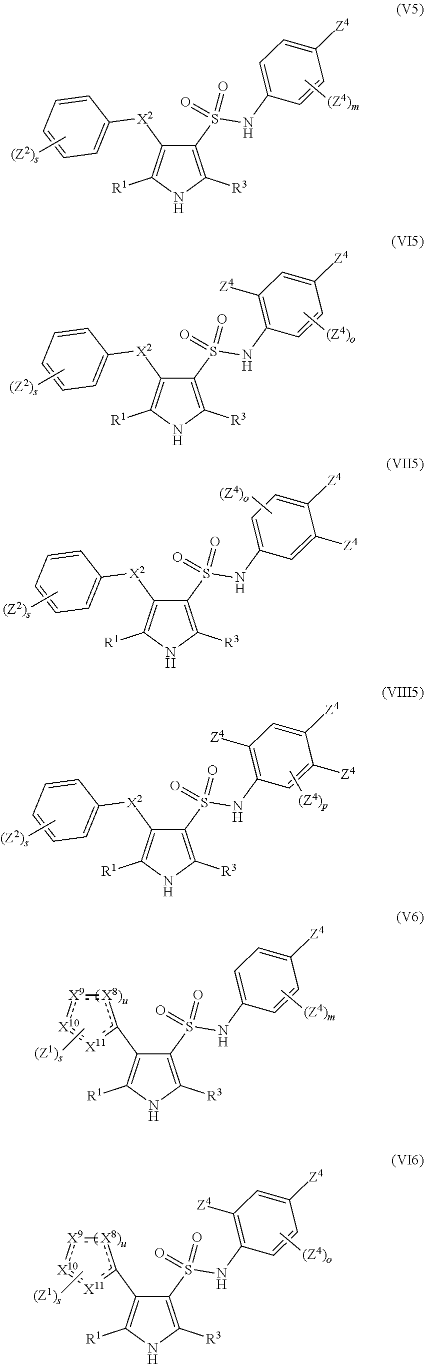

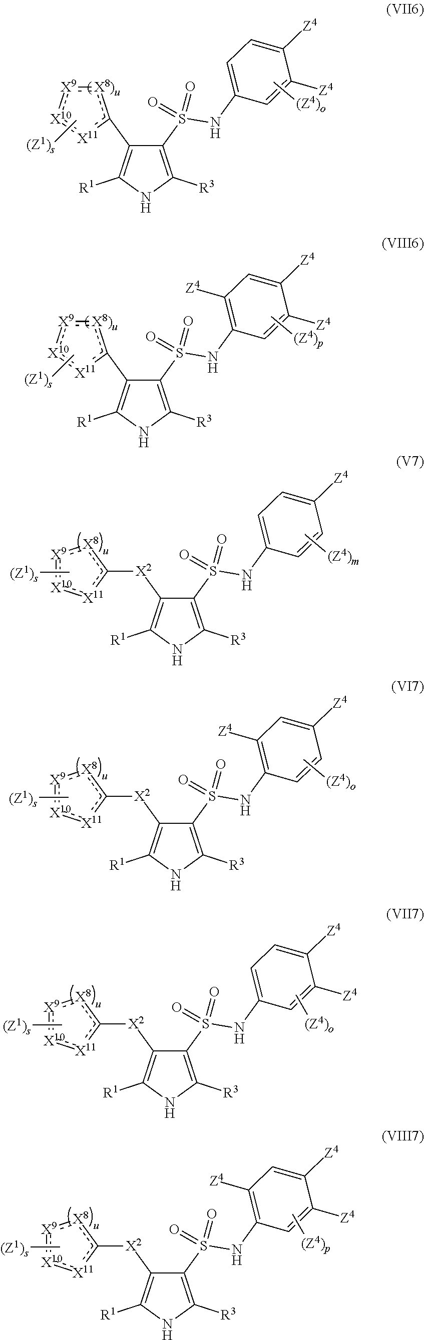

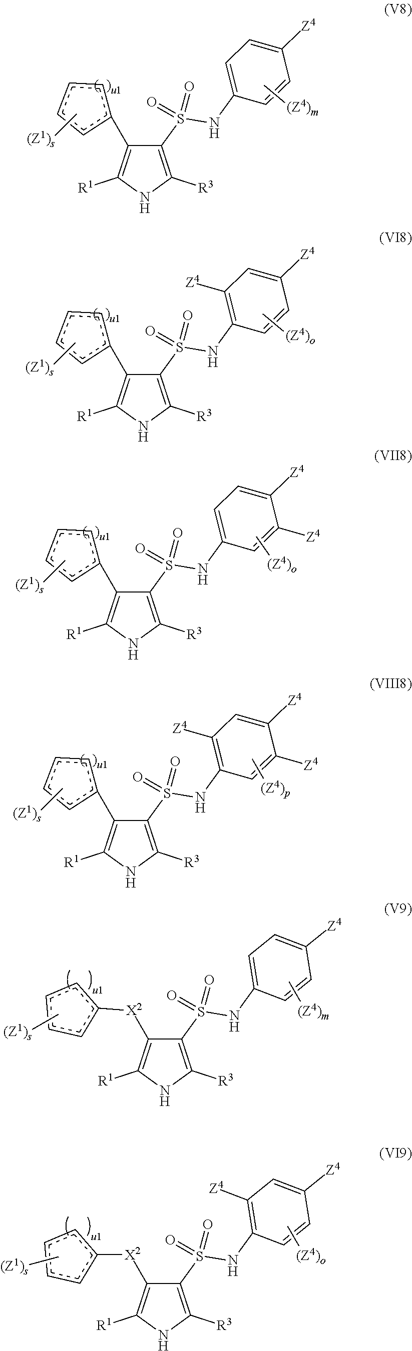

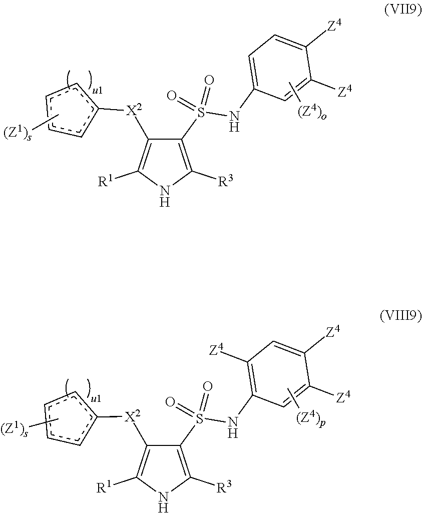

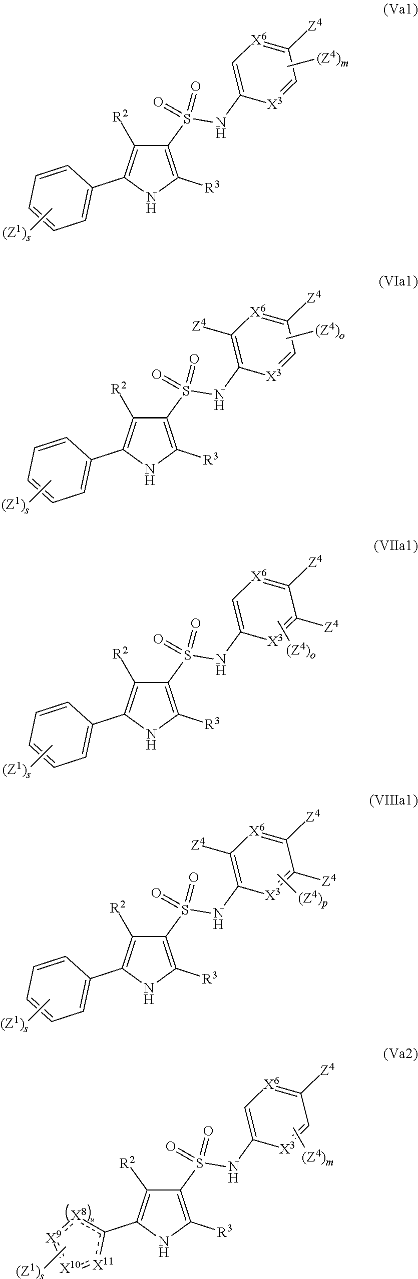

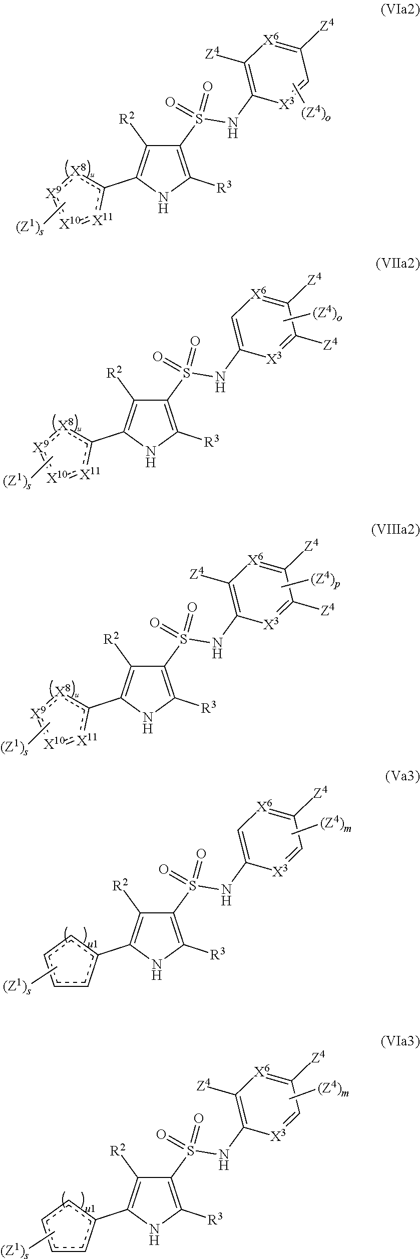

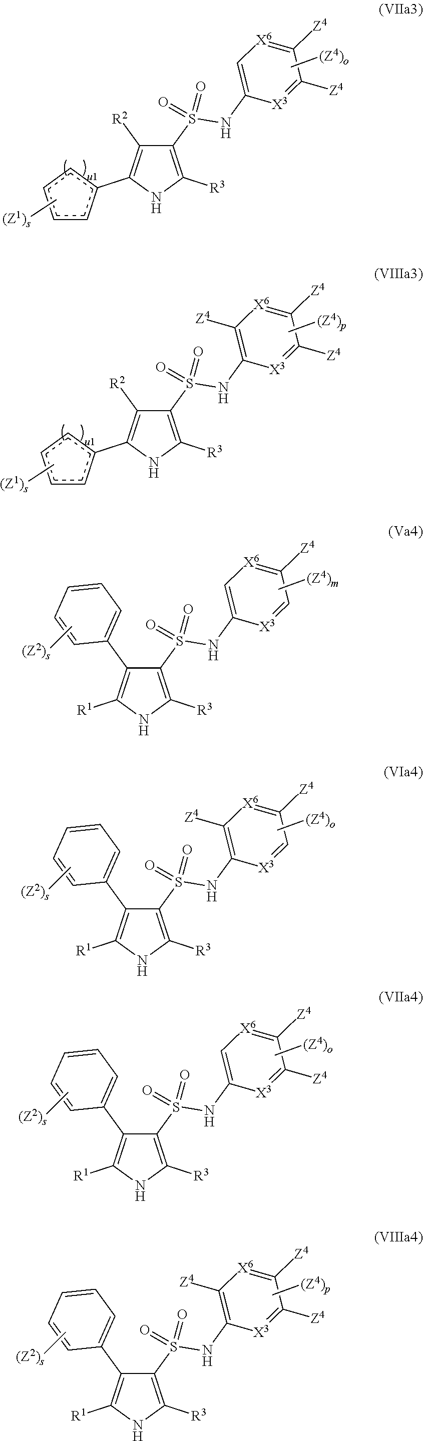

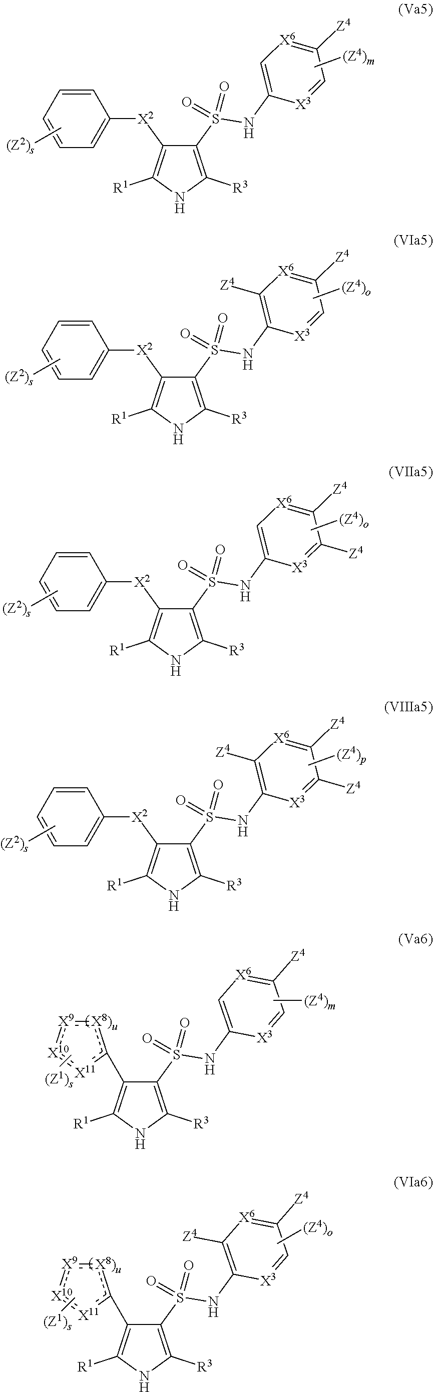

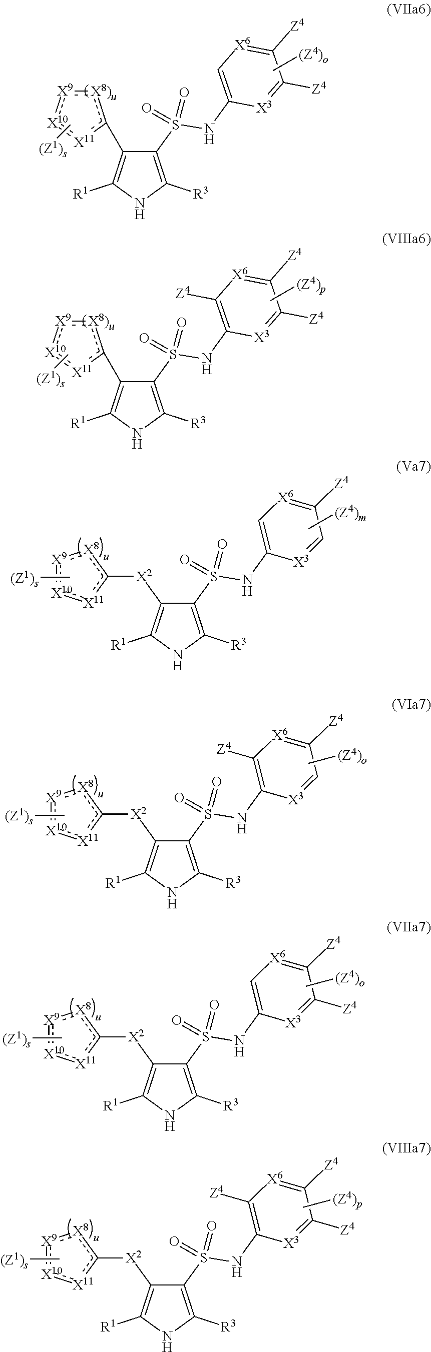

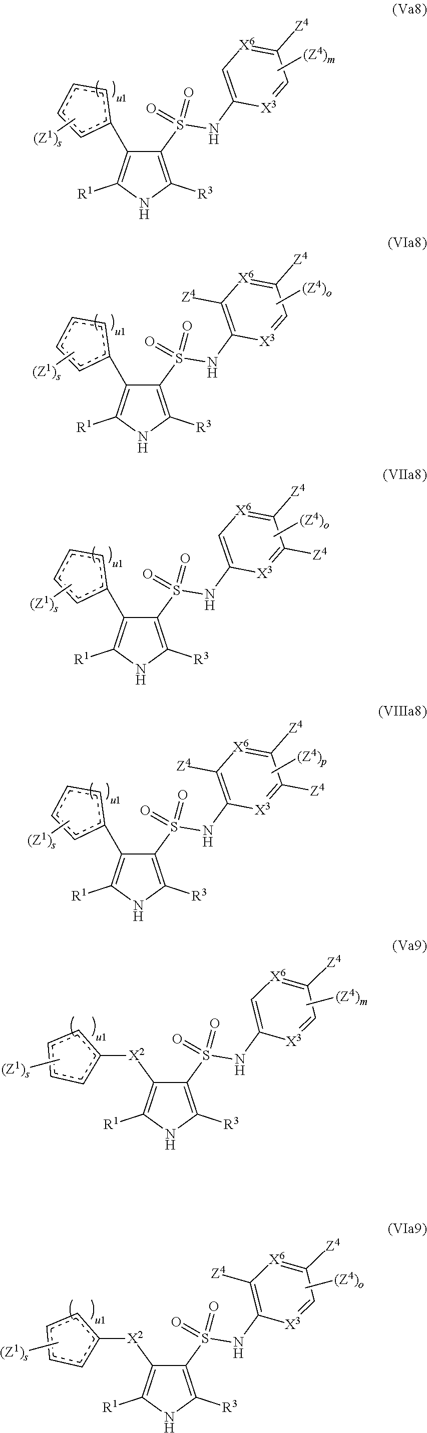

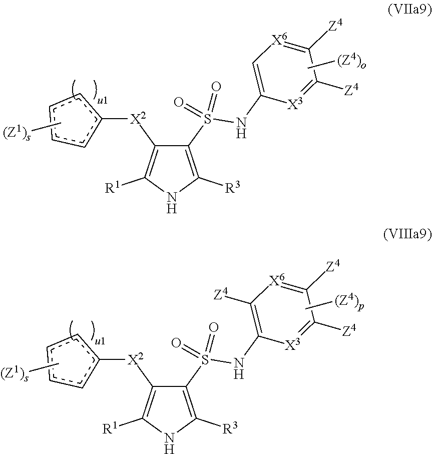

wherein m is an integer selected from 0, 1, 2, or 3;
o is an integer selected from 0, 1, or 2;
p is an integer selected from 0, or 1;
wherein each of X³, X⁶, is independently selected from CH, or N; and at least one of X³, X⁶ is N; wherein each of X⁸, X⁹, X¹⁰, and X¹¹, is independently selected from CH, N, O, or S; and at least one of X⁸, X⁹, X¹⁰, and X¹¹ is selected from N, O, and S; u1 is an integer selected from 0, 1, 2 or 3; u is an integer selected from 1 or 2; n is an integer selected from 1, 2, 3, or 4; s is an integer selected from 0, 1, 2, 3, 4; [symbol] is an optional double bond,
and R¹, R², R³, Z¹, Z⁴ and X² have the same meaning as in any one of statements 1-28.

37. The compound according to any one of statements 1-36, wherein said compound is selected from the group of compounds listed in Table A.

38. The compound according to any one of statements 1-37, wherein said compound comprises at least one isotope selected from the group comprising ²H, ³H, ¹³C, ¹¹C, ¹⁴C, ¹⁵N, ¹⁸O, ¹⁷O, ³¹P, ³²P, ³⁵S, ¹⁸F, ³⁶Cl, ^{99m}Tc, ¹¹¹In, ⁸²Rb, ¹³⁷Cs, ¹²³I, ¹²⁵I, ¹³¹I, ⁶⁷Ga, ¹⁹²Ir and ²⁰¹Tl isotope.

39. A pharmaceutical composition comprising a compound according to any one of statements 1-38, and a pharmaceutical acceptable carrier.

40. A compound according to any one of the preceding statements, or a pharmaceutical composition according to statement 39 for use as a medicine and/or in a diagnostic method.

41. A compound according to any one of statements 1-38, or a pharmaceutical composition according to statement 39, for use in the prevention and/or treatment of GPR17 mediated disorders.

42. A compound according to any one of statements 1-38, or a pharmaceutical composition according to statement 39, for use in the prevention and/or treatment of a disorder or syndrome selected from a myelination disorder and a disorder or syndrome associated with brain tissue damage.

43. A compound for use according to statement 41 or 42, or a pharmaceutical composition for use according to statement 41 or 42, wherein the syndrome or disorder is selected from the group of Multiple Sclerosis (MS) including all its various subforms including clinically isolated syndrome (CIS); optic neuropathies including acute optic neuritis, chronic relapsing inflammatory optic neuritis, neuromyelitis optica (NMO, Devic's disease); acute disseminated encephalomyelitis, acute hemorrhagic leucoencephalitis (AHL); periventricular leukomalacia; demyelination due to autoimmune diseases including anti-MAG peripheral neuropathy and anti-MOG associated spectrum; genetic diseases with white matter pathologies including but not restricted to Sjogren's syndrome, systemic lupus erythematosus, Gaucher's disease, Niemann-Pick disease; leukodystrophies and genetic leukoencephalopathies and adrenoleukodystrophies; demyelination due to viral or bacterial infections; demyelination due to traumatic brain tissue damage and nerve injury; demyelination in response to hypoxia, stroke or ischemia or other cardiovascular diseases; demyelination due to exposure to carbon dioxide, cyanide, vitamin deficiencies or other CNS toxins; central pontine and extrapontine myelinolysis; Schilder's disease; Balo concentric sclerosis; perinatal encephalopathy; neurodegenerative diseases including amyotrophic lateral sclerosis (ALS), Alzheimer's disease (AD), multiple system atrophy, Parkinson's Disease, Niemann-Pick disease, spinocerebellar ataxia (SCA) and Huntington's Disease (HD); psychiatric disorders such as schizophrenia, bipolar disorder, depression and major depressive disorders; and peripheral myelination diseases including acute and chronic peripheral demyelinating neuropathies, Dejerine-Sottas syndrome or Charcot-Marie Tooth disease.

44. A compound for use according to any one of statements 41-43, or a pharmaceutical composition for use according to any one of statements 41-43, wherein the syndrome or disorder is selected from the group of multiple sclerosis (MS) including its various subforms, optic neuritis, neuromyelitis optica (Devic's disease), chronic relapsing inflammatory optic neuritis, acute disseminated encephalomyelitis, acute hemorrhagic leucoencephalitis (AHL), periventricular leukomalacia, demyelination due to viral or bacterial infections, central pontine and extrapontine myelinolysis, demyelination due to traumatic brain tissue damage, demyelination in response to hypoxia, stroke or ischemia or other cardiovascular diseases, demyelination due to exposure to carbon dioxide, cyanide, or other CNS toxins, Schilder's disease, Balo concentric sclerosis, perinatal encephalopathy, neurodegenerative diseases including amyotrophic lateral sclerosis (ALS), Alzheimer's disease (AD), multiple system atrophy, Parkinson's Disease, spinocerebellar ataxia (SCA) and Huntington's Disease, psychiatric disorders such as schizophrenia and bipolar disorder and peripheral myelination diseases including leukodystrophies, peripheral neuropathies, Dejerine-Sottas syndrome or Charcot-Marie-Tooth disease.

45. A compound according to any one of statements 1-38, or a pharmaceutical composition according to statement 39 for use in the prevention and/or treatment of multiple sclerosis (MS).

46. The compound according to statement 38, for use as PET tracers or as SPECT tracers.

47. The compound according to statement 46, for use to perform in vivo diagnosis and/or disease monitoring.

48. The compound according to any one of statements 1-38, for use for the diagnosis and/or monitoring of a GPR17-related disease, preferably of a demyelinating disease, as disclosed herein, preferably in the diagnosis and monitoring of multiple sclerosis.

49. The compound according to any one of statements 1-38, for use to diagnose and/or monitor the expression, distribution and/or activation of the GPR17 receptor either in vivo, e.g., directly in a subject, such as using molecular imaging techniques, or in vitro, such as e.g., by examining any samples such as body fluids or tissues taken from a subject.

50. A kit comprising:
(a) as a first component, a PET or PET tracer based on a compound according to any one of statements 1-37 but having incorporated at least one radionuclide which is suitable for PET or SPECT imaging, or a compound according to statement 38;
(b) as a second component, a therapeutic drug selected from among
i. a compound according to any one of statements 1-37, and having no radionuclide incorporated,
ii. a GPR17 modulating compound which is different from the compounds of the present invention as defined in (i), and
iii. a drug for the treatment of a myelination disease, including but not limited to a drug for use in multiple sclerosis treatment, but having no GPR17 modulating activity; such compounds are known to a person skilled in the art including those examples further described above.

51. A method for the prevention, and/or treatment of a GPR17 mediated disorder, which comprises administering to a patient in need thereof a therapeutically effective amount of a compound according to any one of statements 1-38.

52. A method for the prevention, and/or treatment of a syndrome or disorder selected from a myelination disorder and a disorder or syndrome associated with a brain tissue damage, which comprises administering to a patient in need thereof a therapeutically effective amount of a compound according to any one of statements 1-38.

53. The method according to statement 51 or 52, wherein the syndrome or disorder is the group of Multiple Sclerosis (MS) including all its various subforms including clinically isolated syndrome (CIS); optic neuropathies including acute optic neuritis, chronic relapsing inflammatory optic neuritis, neuromyelitis optica (NMO, Devic's disease); acute disseminated encephalomyelitis, acute hemorrhagic leucoencephalitis (AHL); periventricular leukomalacia; demyelination due to autoimmune diseases including anti-MAG peripheral neuropathy and anti-MOG associated spectrum; genetic diseases with white matter pathologies including but not restricted to Sjogren's syndrome, systemic lupus erythematosus, Gaucher's disease, Niemann-Pick disease; leukodystrophies and genetic leukoencephalopathies and adrenoleukodystrophies; demyelination due to viral or bacterial infections; demyelination due to traumatic brain tissue damage and nerve injury; demyelination in response to hypoxia, stroke or ischemia or other cardiovascular diseases; demyelination due to exposure to carbon dioxide, cyanide, vitamin deficiencies or other CNS toxins; central pontine and extrapontine myelinolysis; Schilder's disease; Balo concentric sclerosis; perinatal encephalopathy; neurodegenerative diseases including amyotrophic lateral sclerosis (ALS), Alzheimer's disease (AD), multiple system atrophy, Parkinson's Disease, Niemann-Pick disease, spinocerebellar ataxia (SCA) and Huntington's Disease (HD); psychiatric disorders such as schizophrenia, bipolar disorder, depression and major depressive disorders; and peripheral myelination diseases including acute and chronic peripheral demyelinating neuropathies, Dejerine-Sottas syndrome or Charcot-Marie Tooth disease.

54. A method according to any one of statements 51-53, wherein the symptom or disorder is associated with a myelination disorder, selected from the group of multiple sclerosis (MS) including its various subforms, optic neuritis, neuromyelitis optica (Devic's disease), chronic relapsing inflammatory optic neuritis, acute disseminated encephalomyelitis, acute hemorrhagic leucoencephalitis (AHL), periventricular leukomalacia, demyelination due to viral infections, central pontine and extrapontine myelinolysis, demyelination due to traumatic brain tissue damage, demyelination in response to hypoxia, stroke or ischemia or other cardiovascular diseases, demyelination due to exposure to carbon dioxide, cyanide, or other CNS toxins, Schilder's disease, Balo concentric sclerosis, perinatal encephalopathy, neurodegenerative diseases including amyotrophic lateral sclerosis (ALS). Alzheimer's disease (AD), multiple system atrophy, Parkinson's Disease, spinocerebellar ataxia (SCA) and Huntington Disease, psychiatric disorders such as schizophrenia and bipolar disorder and peripheral myelination diseases including leukodystrophies, peripheral neuropathies, Dejerine-Sottas syndrome or Charcot-Marie-Tooth disease.

The present invention relates to pyrrolyl-sulfonamide of formula (I) and any subgroups thereof such as compounds of formula (II), (III), (IV), (V), (VI), (VII), (VIII), (Va), (Via), (VIIa), (VIIIa), (IX), (X), (XI), (XII), (XIII), (XIV), (XV), (XVI), (XVII), (XVIII), (XIX), (XX), (II1), (II2), (II3), (III1), (III2), (III3), (III4), (III5), (III6), (III7), (III8), (III9), (IV1), (IV2), (IV3), (IV4), (IV5), (IV6), (IV7), (IV8), (IV9), (V1), (VI1), (VII1), (VIII1), (V2), (VI2), (VII2), (VIII2), (V3), (VI3), (VII3), (VIII113), (V4), (VI4), (VII4), (VIII4), (V5), (VI5), (VII5), (VIII5), (V6), (VI6), (VII6), (VIII6), (V7), (VI7), (VII7), (VIII7), (V8), (VI8), (VII8), (VIII18), (V9), (VI9), (VII9), (VIII9), (Va1), (Via1), (VIIa1), (VIIIa1), (Va2), (Via2), (VIIa2), (VIIIa2), (Va3), (Via3), (VIIa3), (VIIIa3), (Va4), (Via4), (VIIa4), (VIIIa4), (Va5), (Via5), (VIIa5), (VIIIa5), (Va6), (Via6), (VIIa6), (VIIIa6), (Va7), (Via7), (VIIa7), (VIIIa7), (Va8), (Via8), (VIIa8), (VIIIa8), (Va9), (Via9), (VIIa9), or (VIIIa9), as described herein; or an isomer such as a stereoisomer and a tautomer, a stereoisomer, a salt such as a pharmaceutically and/or physiologically acceptable salt, a hydrate, a solvate, a polymorph, a prodrug, an isotope or a co-crystal thereof.

In one embodiment, the present invention relates to a compound of formula (I), or any subgroup thereof, wherein:
R¹ is selected from the group comprising aryl, heteroaryl, cycloalkyl, cycloalkenyl, cycloalkynyl, heterocyclyl, and A¹-X¹—; preferably R¹ is selected from the group comprising aryl, heteroaryl, cycloalkyl, cycloalkenyl, heterocyclyl; and
R² is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl, preferably R² is selected from the group comprising hydrogen, halo, cyano, alkyl, alkenyl, haloalkyl, haloalkenyl, alkoxy, alkenyloxy, alkylthio, alkenylthio, haloalkoxy, alkoxyalkyl, mono or di(alkyl)amino, and mono or di(alkyl)aminoalkyl.

In an alternate embodiment, the present invention relates to a compound of formula (I), or any subgroup thereof, wherein:
R¹ is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl; preferably R¹ is selected from the group comprising hydrogen, halo, cyano, alkyl, alkenyl, haloalkyl, haloalkenyl, alkoxy, alkenyloxy, alkylthio, alkenylthio, haloalkoxy, alkoxyalkyl, mono or di(alkyl)amino, and mono or di(alkyl)aminoalkyl; and
R² is selected from the group comprising aryl, heteroaryl, cycloalkyl, cycloalkenyl, cycloalkynyl, heterocyclyl, and A²-X²—; preferably R² is selected from the group comprising aryl, heteroaryl, cycloalkyl, cycloalkenyl, heterocyclyl, and A²-X²—.

In a preferred embodiment of the invention, the compound of formula (I) is selected from the group of compounds listed in Table A below, or an isomer such as a stereoisomer and a tautomer, a stereoisomer, a salt such as a pharmaceutically and/or physiologically acceptable salt, a hydrate, a solvate, a polymorph, a prodrug, an isotope, or a co-crystal thereof.

TABLE A
Cpd 001 - N-(4-cyano-2-fluoro-phenyl)-2-methyl-5-phenyl-1H-pyrrole-3-sulfonamide
Cpd 002 - N-(4-cyano-2-fluoro-phenyl)-5-phenyl-1H-pyrrole-3-sulfonamide
Cpd 003 - N-(4-bromo-2,5-difluoro-phenyl)-5-phenyl-1H-pyrrole-3-sulfonamide
Cpd 004 - N-(4-cyano-2-fluoro-phenyl)-5-(3-fluorophenyl)-1H-pyrrole-3-sulfonamide
Cpd 005 - N-(4-cyano-2-fluoro-phenyl)-5-(m-tolyl)-1H-pyrrole-3-sulfonamide
Cpd 006 - 5-(3-chlorophenyl)-N-(4-cyano-2-fluoro-phenyl)-1H-pyrrole-3-sulfonamide
Cpd 007 - N-(4-cyano-2-fluoro-phenyl)-5-(4-fluorophenyl)-1H-pyrrole-3-sulfonamide
Cpd 008 - N-(4-cyano-2-fluoro-phenyl)-5-(p-tolyl)-1H-pyrrole-3-sulfonamide
Cpd 009 - N-(4-cyano-2-fluoro-phenyl)-4-phenyl-1H-pyrrole-3-sulfonamide
Cpd 010 - N-(4-cyano-2-fluoro-phenyl)-5-(3-methoxyphenyl)-1H-pyrrole-3-sulfonamide
Cpd 011 - 5-(4-chlorophenyl)-N-(4-cyano-2-fluoro-phenyl)-1H-pyrrole-3-sulfonamide
Cpd 012 - N-(4-cyano-2-fluoro-phenyl)-5-(4-methoxyphenyl)-1H-pyrrole-3-sulfonamide
Cpd 013 - 5-benzoyl-N-(4-cyano-2-fluoro-phenyl)-1H-pyrrole-3-sulfonamide
Cpd 014 - N-(4-cyano-2-fluoro-phenyl)-5-(2-fluorophenyl)-1H-pyrrole-3-sulfonamide
Cpd 015 - N-(4-cyano-2-fluoro-phenyl)-5-(o-tolyl)-1H-pyrrole-3-sulfonamide
Cpd 016 - 5-(2-chlorophenyl)-N-(4-cyano-2-fluoro-phenyl)-1H-pyrrole-3-sulfonamide
Cpd 017 - N-(4-cyano-2-fluoro-phenyl)-5-(2-methoxyphenyl)-1H-pyrrole-3-sulfonamide
Cpd 018 - N-(4-cyano-2-fluoro-phenyl)-5-cyclopentyl-1H-pyrrole-3-sulfonamide
Cpd 019 - 5-benzyl-N-(4-cyano-2-fluoro-phenyl)-1H-pyrrole-3-sulfonamide
Cpd 020 - N-(4-cyano-2-fluoro-phenyl)-2-fluoro-5-phenyl-1H-pyrrole-3-sulfonamide
Cpd 021 - N-(4-cyano-2-fluoro-phenyl)-5-[3-(trifluoromethyl)phenyl]-1H-pyrrole-3-sulfonamide
Cpd 022 - N-(4-cyano-2-fluoro-phenyl)-5-(3-isopropylphenyl)-1H-pyrrole-3-sulfonamide
Cpd 023 - N-(4-cyano-2-fluoro-phenyl)-4-fluoro-5-phenyl-1H-pyrrole-3-sulfonamide
Cpd 024 - N-(4-cyano-2-fluoro-phenyl)-5-(4-pyridyl)-1H-pyrrole-3-sulfonamide
Cpd 025 - N-(4-cyano-2-fluoro-phenyl)-5-(3-cyanophenyl)-1H-pyrrole-3-sulfonamide
Cpd 026 - N-(4-cyano-2-fluoro-phenyl)-5-(2-pyridyl)-1H-pyrrole-3-sulfonamide
Cpd 027 - N-(4-cyano-2-fluoro-phenyl)-5-(3-pyridyl)-1H-pyrrole-3-sulfonamide
Cpd 028 - N-(4-cyano-2,5-difluoro-phenyl)-5-phenyl-1H-pyrrole-3-sulfonamide
Cpd 029 - N-(4-cyano-2-methyl-phenyl)-5-phenyl-1H-pyrrole-3-sulfonamide
Cpd 030 - N-(4-cyano-2-methoxy-phenyl)-5-phenyl-1H-pyrrole-3-sulfonamide
Cpd 031 - N-(4-cyano-3-methyl-phenyl)-5-phenyl-1H-pyrrole-3-sulfonamide
Cpd 032 - N-(2-fluoro-4-methoxy-phenyl)-5-phenyl-1H-pyrrole-3-sulfonamide
Cpd 033 - N-(4-cyano-2-fluoro-phenyl)-4-methyl-5-phenyl-1H-pyrrole-3-sulfonamide
Cpd 034 - 4-benzyl-N-(4-cyano-2-fluoro-phenyl)-1H-pyrrole-3-sulfonamide
Cpd 035 - N-(4-cyano-3-fluoro-phenyl)-5-phenyl-1H-pyrrole-3-sulfonamide
Cpd 036 - N-(2-chloro-4-cyano-phenyl)-5-phenyl-1H-pyrrole-3-sulfonamide
Cpd 037 - N-(5-cyano-2-pyridyl)-5-phenyl-1H-pyrrole-3-sulfonamide
Cpd 038 - N-(4-cyano-2-fluoro-phenyl)-5-(2,4-difluorophenyl)-1H-pyrrole-3-sulfonamide
Cpd 039 - 5-(3-chloro-2-fluoro-phenyl)-N-(4-cyano-2-fluoro-phenyl)-1H-pyrrole-3-sulfonamide
Cpd 040 - N-(4-cyano-2-fluoro-phenyl)-5-(2,3-difluorophenyl)-1H-pyrrole-3-sulfonamide
Cpd 041 - N-(4-cyano-2-fluoro-phenyl)-5-(2,5-difluorophenyl)-1H-pyrrole-3-sulfonamide
Cpd 042 - N-(4-cyano-2-fluoro-phenyl)-5-(2-fluoro-3-methyl-phenyl)-1H-pyrrole-3-sulfonamide
Cpd 043 - N-(4-cyano-2-fluoro-phenyl)-5-(2-fluoro-4-methyl-phenyl)-1H-pyrrole-3-sulfonamide
Cpd 044 - N-(4-cyano-2-fluoro-phenyl)-5-(2-fluoro-5-methyl-phenyl)-1H-pyrrole-3-sulfonamide
Cpd 045 - 5-(4-chloro-2-fluoro-phenyl)-N-(4-cyano-2-fluoro-phenyl)-1H-pyrrole-3-sulfonamide
Cpd 046 - 5-(5-chloro-2-fluoro-phenyl)-N-(4-cyano-2-fluoro-phenyl)-1H-pyrrole-3-sulfonamide
Cpd 047 - 5-(2-fluorophenyl)-N-(2,4,5-trifluorophenyl)-1H-pyrrole-3-sulfonamide
Cpd 048 - N-(5-chloro-3-fluoro-2-pyridyl)-5-(2-fluorophenyl)-1H-pyrrole-3-sulfonamide
Cpd 049 - N-(1,3-benzodioxol-4-yl)-5-(2-fluorophenyl)-1H-pyrrole-3-sulfonamide
Cpd 050 - N-(2,1,3-benzothiadiazol-4-yl)-5-(2-fluorophenyl)-1H-pyrrole-3-sulfonamide
Cpd 051 - N-(2,5-difluorophenyl)-5-(2-fluorophenyl)-1H-pyrrole-3-sulfonamide
Cpd 052 - N-(4-chloro-2-fluoro-phenyl)-5-(2-fluorophenyl)-1H-pyrrole-3-sulfonamide
Cpd 053 - N-[4-(cyanomethyl)-2-fluoro-phenyl]-5-(2-fluorophenyl)-1H-pyrrole-3-sulfonamide
Cpd 054 - N-(2,4-difluorophenyl)-5-(2-fluorophenyl)-1H-pyrrole-3-sulfonamide
Cpd 055 - 5-(2-fluorophenyl)-N-[2-fluoro-4-(trifluoromethyl)phenyl]-1H-pyrrole-3-sulfonamide
Cpd 056 - N-(2,3-difluorophenyl)-5-(2-fluorophenyl)-1H-pyrrole-3-sulfonamide
Cpd 057 - N-(2-fluoro-4-methyl-phenyl)-5-(2-fluorophenyl)-1H-pyrrole-3-sulfonamide
Cpd 058 - N-(3-chloro-4-cyano-phenyl)-5-(2-fluorophenyl)-1H-pyrrole-3-sulfonamide
Cpd 059 - N-(4-cyano-3-methoxy-phenyl)-5-(2-fluorophenyl)-1H-pyrrole-3-sulfonamide
Cpd 060 - 5-(2-fluorophenyl)-N-[3-methoxy-5-(trifluoromethyl)-2-pyridyl]-1H-pyrrole-3-sulfonamide
Cpd 061 - N-[5-(cyanomethyl)-3-methoxy-2-pyridyl]-5-phenyl-1H-pyrrole-3-sulfonamide
Cpd 062 - N-(5-bromo-3-methoxy-2-pyridyl)-5-phenyl-1H-pyrrole-3-sulfonamide
Cpd 063 - N-(4-cyano-5-fluoro-2-methoxy-phenyl)-5-phenyl-1H-pyrrole-3-sulfonamide
Cpd 064 - N-(4-cyano-2,6-difluoro-phenyl)-5-phenyl-1H-pyrrole-3-sulfonamide
Cpd 065 - N-[4-(cyanomethoxy)-2,5-difluoro-phenyl]-5-phenyl-1H-pyrrole-3-sulfonamide
Cpd 066 - N-(4-cyano-2-fluoro-phenyl)-5-(3-fluoro-4-pyridyl)-1H-pyrrole-3-sulfonamide
Cpd 067 - 4-benzyl-N-(4-cyano-2-fluoro-phenyl)-5-methyl-1H-pyrrole-3-sulfonamide
Cpd 068 - N-(5-chloro-3-fluoro-2-pyridyl)-5-phenyl-1H-pyrrole-3-sulfonamide
Cpd 069 - N-(1,3-benzodioxol-4-yl)-5-phenyl-1H-pyrrole-3-sulfonamide
Cpd 070 - N-(2,5-difluoro-4-methyl-phenyl)-5-phenyl-1H-pyrrole-3-sulfonamide
Cpd 071 - N-[2,5-difluoro-4-(trifluoromethyl)phenyl]-5-phenyl-1H-pyrrole-3-sulfonamide
Cpd 072 - N-(4-cyano-3-fluoro-phenyl)-5-(2-fluorophenyl)-1H-pyrrole-3-sulfonamide
Cpd 073 - N-[2,5-difluoro-4-(trifluoromethyl)phenyl]-5-(2-fluorophenyl)-1H-pyrrole-3-sulfonamide
Cpd 074 - N-(4-cyano-2,5-difluoro-phenyl)-5-(2-fluorophenyl)-1H-pyrrole-3-sulfonamide
Cpd 075 - N-(2,5-difluoro-4-methyl-phenyl)-5-(2-fluorophenyl)-1H-pyrrole-3-sulfonamide
Cpd 076 - N-(4-cyano-2,5-difluoro-phenyl)-4-fluoro-5-phenyl-1H-pyrrole-3-sulfonamide
Cpd 077 - N-(4-cyano-2,5-difluoro-phenyl)-2-fluoro-5-phenyl-1H-pyrrole-3-sulfonamide
Cpd 078 - N-(4-cyano-2-fluoro-phenyl)-5-(2-methylpyrazol-3-yl)-1H-pyrrole-3-sulfonamide
Cpd 079 - N-(4-cyano-2-fluoro-phenyl)-5-[2-(trifluoromethyl)phenyl]-1H-pyrrole-3-sulfonamide
Cpd 080 - N-(4-cyano-2-fluoro-phenyl)-5-[2-(methoxymethyl)phenyl]-1H-pyrrole-3-sulfonamide
Cpd 081 - N-(4-cyano-2-fluoro-phenyl)-5-(1-methylpyrazol-3-yl)-1H-pyrrole-3-sulfonamide
Cpd 082 - N-(4-cyano-2-fluoro-phenyl)-5-(2-fluoro-6-methyl-phenyl)-1H-pyrrole-3-sulfonamide
Cpd 083 - N-(4-cyano-2-fluoro-phenyl)-5-(2-fluoro-6-methoxy-phenyl)-1H-pyrrole-3-sulfonamide
Cpd 084 - N-(4-cyano-2-fluoro-phenyl)-5-(2-fluoro-5-methoxy-phenyl)-1H-pyrrole-3-sulfonamide
Cpd 085 - N-(4-cyano-2-fluoro-phenyl)-5-(2,6-dimethylphenyl)-1H-pyrrole-3-sulfonamide
Cpd 086 - N-(4-cyano-2-fluoro-phenyl)-5-(2-fluoro-4-methoxy-phenyl)-1H-pyrrole-3-sulfonamide
Cpd 087 - N-(4-cyano-2-fluoro-phenyl)-5-(2-fluoro-3-methoxy-phenyl)-1H-pyrrole-3-sulfonamide
Cpd 088 - N-(4-cyano-2-fluoro-phenyl)-5-(2,4,5-trifluorophenyl)-1H-pyrrole-3-sulfonamide
Cpd 089 - N-(4-cyano-2-fluoro-phenyl)-5-(4-fluoro-2-methoxy-3-pyridyl)-1H-pyrrole-3-sulfonamide
Cpd 090 - N-(4-cyano-2-fluoro-phenyl)-5-(3-fluoro-2-pyridyl)-1H-pyrrole-3-sulfonamide
Cpd 091 - N-(4-cyano-2-fluoro-phenyl)-5-(2,6-difluorophenyl)-1H-pyrrole-3-sulfonamide
Cpd 093 - N-(4-cyano-2-fluoro-phenyl)-5-[2-(difluoromethyl)phenyl]-1H-pyrrole-3-sulfonamide
Cpd 094 - N-(4-cyano-2-fluoro-phenyl)-5-pyrimidin-2-yl-1H-pyrrole-3-sulfonamide
Cpd 095 - N-(4-cyano-2-fluoro-phenyl)-5-(2,6-dimethoxyphenyl)-1H-pyrrole-3-sulfonamide
Cpd 096 - N,5-bis(4-cyano-2-fluoro-phenyl)-1H-pyrrole-3-sulfonamide
Cpd 097 - N-(4-cyano-2-fluoro-phenyl)-5-(2,3,5-trifluorophenyl)-1H-pyrrole-3-sulfonamide
Cpd 098 - N-(4-cyano-2-fluoro-phenyl)-5-cyclobutyl-1H-pyrrole-3-sulfonamide
Cpd 099 - 5-phenyl-N-[6-(trifluoromethyl)-3-pyridyl]-1H-pyrrole-3-sulfonamide
Cpd 100 - N-(2-methyl-3-pyridyl)-5-phenyl-1H-pyrrole-3-sulfonamide
Cpd 101 - N-(2,6-dimethyl-3-pyridyl)-5-phenyl-1H-pyrrole-3-sulfonamide
Cpd 102 - N-(4-cyano-2-fluoro-phenyl)-5-(2-thienyl)-1H-pyrrole-3-sulfonamide
Cpd 103 - N-(4-cyano-2-fluoro-phenyl)-5-(3-thienyl)-1H-pyrrole-3-sulfonamide
Cpd 104 - N-(4-cyano-2-fluoro-phenyl)-5-(3,5-difluorophenyl)-1H-pyrrole-3-sulfonamide
Cpd 105 - N-(4-cyano-2-fluoro-phenyl)-5-(2-fluoro-3-pyridyl)-1H-pyrrole-3-sulfonamide
Cpd 106 - N-(4-cyano-2-fluoro-phenyl)-5-(2-cyanophenyl)-1H-pyrrole-3-sulfonamide
Cpd 107 - N-(4-cyano-2-fluoro-phenyl)-5-[2-fluoro-3-(hydroxymethyl)phenyl]-1H-pyrrole-3-sulfonamide
Cpd 108 - N-(4-cyano-2-fluoro-phenyl)-5-[2-fluoro-5-(hydroxymethyl)phenyl]-1H-pyrrole-3-sulfonamide
Cpd 109 - 5-(4-fluorophenyl)-N-(2,4,5-trifluorophenyl)-1H-pyrrole-3-sulfonamide
Cpd 110 - 5-(3-fluorophenyl)-N-(2,4,5-trifluorophenyl)-1H-pyrrole-3-sulfonamide
Cpd 111 - 5-(o-tolyl)-N-(2,4,5-trifluorophenyl)-1H-pyrrole-3-sulfonamide
Cpd 112 - 5-phenyl-N-(6-quinolyl)-1H-pyrrole-3-sulfonamide
Cpd 113 - N-[2,5-difluoro-4-(trifluoromethyl)phenyl]-5-(4-fluorophenyl)-1H-pyrrole-3-sulfonamide
Cpd 114 - N-[2,5-difluoro-4-(trifluoromethyl)phenyl]-5-(3-fluorophenyl)-1H-pyrrole-3-sulfonamide
Cpd 115 - N-[2-fluoro-4-(2-fluoroethoxy)phenyl]-5-(2-fluorophenyl)-1H-pyrrole-3-sulfonamide
Cpd 116 - N-[6-(difluoromethoxy)-5-fluoro-2-methoxy-3-pyridyl]-5-(2-fluorophenyl)-1H-pyrrole-3-sulfonamide
Cpd 117 - N-[2,5-difluoro-4-(trifluoromethyl)phenyl]-5-(2-fluoro-5-methoxy-phenyl)-1H-pyrrole-3-sulfonamide
Cpd 118 - N-[2,5-difluoro-4-(trifluoromethyl)phenyl]-5-(o-tolyl)-1H-pyrrole-3-sulfonamide
Cpd 119 - 5-(2,6-difluorophenyl)-N-[2,5-difluoro-4-(trifluoromethyl)phenyl]-1H-pyrrole-3-sulfonamide
Cpd 120 - N-(2,4-difluorophenyl)-5-(3-fluorophenyl)-1H-pyrrole-3-sulfonamide
Cpd 121 - N-(2,4-difluorophenyl)-5-(4-fluorophenyl)-1H-pyrrole-3-sulfonamide
Cpd 122 - N-(2,4-difluorophenyl)-5-(2-pyridyl)-1H-pyrrole-3-sulfonamide
Cpd 123 - N-[2-fluoro-4-(trifluoromethyl)phenyl]-5-(2-pyridyl)-1H-pyrrole-3-sulfonamide
Cpd 124 - N-[2,5-difluoro-4-(trifluoromethyl)phenyl]-5-(2-pyridyl)-1H-pyrrole-3-sulfonamide
Cpd 125 - N-(3-fluorophenyl)-5-(2-pyridyl)-1H-pyrrole-3-sulfonamide
Cpd 126 - N-(6-chloro-2-methyl-3-pyridyl)-5-phenyl-1H-pyrrole-3-sulfonamide
Cpd 127 - N-(2-chloro-6-methyl-3-pyridyl)-5-phenyl-1H-pyrrole-3-sulfonamide
Cpd 128 - N-(2-methoxy-6-methyl-3-pyridyl)-5-phenyl-1H-pyrrole-3-sulfonamide
Cpd 129 - N-(2,4-dimethyl-3-pyridyl)-5-phenyl-1H-pyrrole-3-sulfonamide
Cpd 130 - N-[2-fluoro-4-(2-methoxyethoxy)phenyl]-5-(2-fluorophenyl)-1H-pyrrole-3-sulfonamide
Cpd 131 - 5-(2,6-difluorophenyl)-N-(2,4,5-trifluorophenyl)-1H-pyrrole-3-sulfonamide
Cpd 132 - 5-cyclopentyl-N-[2,5-difluoro-4-(trifluoromethyl)phenyl]-1H-pyrrole-3-sulfonamide
Cpd 133 - N-(3,5-difluoro-2-pyridyl)-5-(2-fluorophenyl)-1H-pyrrole-3-sulfonamide
Cpd 134 - N-(5-chloro-3-methoxy-2-pyridyl)-5-phenyl-1H-pyrrole-3-sulfonamide
Cpd 135 - N-[2-methyl-6-(trifluoromethyl)-3-pyridyl]-5-phenyl-1H-pyrrole-3-sulfonamide
Cpd 136 - N-(3-fluoro-5-methyl-2-pyridyl)-5-phenyl-1H-pyrrole-3-sulfonamide
Cpd 137 - 5-phenyl-N-(2,4,5-trifluorophenyl)-1H-pyrrole-3-sulfonamide
Cpd 138 - 5-phenyl-N-[5-(trifluoromethyl)-2-pyridyl]-1H-pyrrole-3-sulfonamide
Cpd 139 - 5-phenyl-N-[4-(trifluoromethyl)-2-pyridyl]-1H-pyrrole-3-sulfonamide
Cpd 140 - N-[3-fluoro-5-(trifluoromethyl)-2-pyridyl]-5-phenyl-1H-pyrrole-3-sulfonamide
Cpd 141 - N-(2,2-difluoro-1,3-benzodioxol-4-yl)-5-phenyl-1H-pyrrole-3-sulfonamide
Cpd 142 - N-(6-chloro-4-fluoro-3-pyridyl)-5-(2-fluorophenyl)-1H-pyrrole-3-sulfonamide
Cpd 143 - N-[2-fluoro-4-(trifluoromethoxy)phenyl]-5-phenyl-1H-pyrrole-3-sulfonamide
Cpd 144 - N-[2-methoxy-4-(trifluoromethyl)phenyl]-5-phenyl-1H-pyrrole-3-sulfonamide
Cpd 145 - N-(4-chloro-2,5-difluoro-phenyl)-5-phenyl-1H-pyrrole-3-sulfonamide
Cpd 146 - N-(2-chloro-5-fluoro-4-pyridyl)-5-phenyl-1H-pyrrole-3-sulfonamide
Cpd 147 - N-[2-methoxy-6-(trifluoromethyl)-3-pyridyl]-5-phenyl-1H-pyrrole-3-sulfonamide
Cpd 148 - N-[2,5-difluoro-4-(trifluoromethyl)phenyl]-5-(3-fluoro-2-pyridyl)-1H-pyrrole-3-sulfonamide
Cpd 149 - N-(1,3-benzodioxol-4-yl)-5-(2-pyridyl)-1H-pyrrole-3-sulfonamide
Cpd 150 - N-[2,5-difluoro-4-(trifluoromethyl)phenyl]-5-pyrimidin-2-yl-1H-pyrrole-3-sulfonamide
Cpd 151 - 4-benzyl-N-(2,4-difluorophenyl)-1H-pyrrole-3-sulfonamide
Cpd 152 - 4-benzyl-N-[6-(trifluoromethyl)-3-pyridyl]-1H-pyrrole-3-sulfonamide
Cpd 153 - 4-benzyl-N-(4-chloro-2-fluoro-phenyl)-1H-pyrrole-3-sulfonamide
Cpd 154 - N-[2-methoxy-4-(trifluoromethyl)phenyl]-5-(2-pyridyl)-1H-pyrrole-3-sulfonamide
Cpd 155 - N-[2,5-difluoro-4-(trifluoromethyl)phenyl]-5-(3-methyl-2-pyridyl)-1H-pyrrole-3-sulfonamide
Cpd 156 - N-[2,5-difluoro-4-(trifluoromethyl)phenyl]-5-(1-methylimidazol-2-yl)-1H-pyrrole-3-sulfonamide
Cpd 157 - N-[2-fluoro-4-(trifluoromethoxy)phenyl]-5-(2-pyridyl)-1H-pyrrole-3-sulfonamide
Cpd 158 - 4-benzyl-N-[2,5-difluoro-4-(trifluoromethyl)phenyl]-1H-pyrrole-3-sulfonamide
Cpd 159 - 4-benzyl-2-chloro-N-(4-cyano-2-fluoro-phenyl)-1H-pyrrole-3-sulfonamide
Cpd 160 - N-(2,4-difluorophenyl)-5-(3-fluoro-2-pyridyl)-1H-pyrrole-3-sulfonamide
Cpd 161 - 4-benzyl-N-(4-cyano-2-methoxy-phenyl)-1H-pyrrole-3-sulfonamide
Cpd 162 - 4-benzyl-N-(4-cyanophenyl)-1H-pyrrole-3-sulfonamide
Cpd 163 - 4-benzyl-N-(2-fluorophenyl)-1H-pyrrole-3-sulfonamide
Cpd 164 - N-(5-chloro-3-fluoro-2-pyridyl)-5-(2-pyridyl)-1H-pyrrole-3-sulfonamide
Cpd 165 - N-[4-(cyanomethyl)-2-methoxy-phenyl]-5-phenyl-1H-pyrrole-3-sulfonamide
Cpd 166 - N-(5-cyano-3-fluoro-2-pyridyl)-5-phenyl-1H-pyrrole-3-sulfonamide
Cpd 167 - N-(5-bromo-3-methoxy-2-pyridyl)-5-(2-pyridyl)-1H-pyrrole-3-sulfonamide
Cpd 168 - N-(5-bromo-3-fluoro-2-pyridyl)-5-phenyl-1H-pyrrole-3-sulfonamide
Cpd 169 - N-[5-(cyanomethyl)-3-fluoro-2-pyridyl]-5-phenyl-1H-pyrrole-3-sulfonamide
Cpd 170 - N-[2,5-difluoro-4-(trifluoromethyl)phenyl]-5-(3-methoxy-2-pyridyl)-1H-pyrrole-3-sulfonamide
Cpd 171 - N-(4-cyano-2-fluoro-phenyl)-4-[(3-fluorophenyl)methyl]-1H-pyrrole-3-sulfonamide
Cpd 172 - N-(4-bromo-2,5-difluoro-phenyl)-5-(2-pyridyl)-1H-pyrrole-3-sulfonamide
Cpd 173 - N-(2,5-difluoro-4-methyl-phenyl)-5-(2-pyridyl)-1H-pyrrole-3-sulfonamide
Cpd 174 - N-(2,5-difluoro-4-phenyl-phenyl)-5-(2-pyridyl)-1H-pyrrole-3-sulfonamide
Cpd 175 - N-[3-methoxy-5-(trifluoromethyl)-2-pyridyl]-5-phenyl-1H-pyrrole-3-sulfonamide
Cpd 176 - 5-(2-fluorophenyl)-N-[3-fluoro-5-(trifluoromethyl)-2-pyridyl]-1H-pyrrole-3-sulfonamide
Cpd 177 - N-(4-cyano-2-fluoro-phenyl)-4-[(4-fluorophenyl)methyl]-1H-pyrrole-3-sulfonamide
Cpd 178 - N-(4-cyclopropyl-2,5-difluoro-phenyl)-5-(2-pyridyl)-1H-pyrrole-3-sulfonamide
Cpd 179 - 5-(2-pyridyl)-N-(2,4,5-trifluorophenyl)-1H-pyrrole-3-sulfonamide
Cpd 180 - N-[2,5-difluoro-4-(trifluoromethyl)phenyl]-5-(2-fluoro-6-methoxy-phenyl)-1H-pyrrole-3-sulfonamide
Cpd 181 - N-(4-chloro-2-fluoro-phenyl)-5-(2-pyridyl)-1H-pyrrole-3-sulfonamide
Cpd 182 - N-(4-cyano-2-fluoro-phenyl)-4-[(2-fluorophenyl)methyl]-1H-pyrrole-3-sulfonamide
Cpd 183 - N-(2,5-difluoro-4-methoxy-phenyl)-5-(2-pyridyl)-1H-pyrrole-3-sulfonamide
Cpd 184 - N-(4-cyano-2-fluoro-phenyl)-4-(3-pyridylmethyl)-1H-pyrrole-3-sulfonamide
Cpd 185 - N-(4-ethynyl-2,5-difluoro-phenyl)-5-(2-pyridyl)-1H-pyrrole-3-sulfonamide
Cpd 186 - N-[2-fluoro-6-(trifluoromethyl)-3-pyridyl]-5-phenyl-1H-pyrrole-3-sulfonamide
Cpd 187 - N-(7-fluoro-6-quinolyl)-5-phenyl-1H-pyrrole-3-sulfonamide
Cpd 188 - N-[6-(difluoromethoxy)-2-fluoro-3-pyridyl]-5-phenyl-1H-pyrrole-3-sulfonamide
Cpd 189 - N-[2,5-difluoro-4-(trifluoromethyl)phenyl]-5-(5-fluoro-2-pyridyl)-1H-pyrrole-3-sulfonamide
Cpd 190 - N-[3-fluoro-4-(trifluoromethyl)phenyl]-5-(2-pyridyl)-1H-pyrrole-3-sulfonamide
Cpd 191 - N-[2,5-difluoro-4-(trifluoromethyl)phenyl]-5-(2-methoxy-3-pyridyl)-1H-pyrrole-3-sulfonamide
Cpd 192 - 4-benzyl-N-(5-chloro-3-fluoro-2-pyridyl)-1H-pyrrole-3-sulfonamide
Cpd 193 - 4-benzyl-N-(4-bromo-2,5-difluoro-phenyl)-1H-pyrrole-3-sulfonamide
Cpd 194 - 4-[(2-chlorophenyl)methyl]-N-(4-cyano-2-fluoro-phenyl)-1H-pyrrole-3-sulfonamide
Cpd 195 - 4-benzoyl-N-(4-cyano-2-fluoro-phenyl)-1H-pyrrole-3-sulfonamide
Cpd 196 - N-(2-fluoro-5-methoxy-phenyl)-5-(2-pyridyl)-1H-pyrrole-3-sulfonamide
Cpd 197 - 5-(5-chloro-2-pyridyl)-N-[2,5-difluoro-4-(trifluoromethyl)phenyl]-1H-pyrrole-3-sulfonamide
Cpd 198 - N-[3-methoxy-5-(trifluoromethyl)-2-pyridyl]-5-(2-pyridyl)-1H-pyrrole-3-sulfonamide
Cpd 199 - N-[5-fluoro-2-(trifluoromethyl)-4-pyridyl]-5-phenyl-1H-pyrrole-3-sulfonamide
Cpd 200 - N-(4-acetyl-2-fluoro-phenyl)-5-(2-pyridyl)-1H-pyrrole-3-sulfonamide
Cpd 201 - N-[5-(cyanomethyl)-3-fluoro-2-pyridyl]-5-(2-pyridyl)-1H-pyrrole-3-sulfonamide
Cpd 202 - N-(4-chloro-2,5-difluoro-phenyl)-5-(2-pyridyl)-1H-pyrrole-3-sulfonamide
Cpd 203 - ethyl 3-fluoro-4-[5-(2-pyridyl)-1H-pyrrol-3-yl]sulfonylamino]benzoate
Cpd 204 - 5-(3-chloro-2-pyridyl)-N-[2,5-difluoro-4-(trifluoromethyl)phenyl]-1H-pyrrole-3-sulfonamide
Cpd 205 - N-[4-(difluoromethoxy)-2,5-difluoro-phenyl]-5-(2-pyridyl)-1H-pyrrole-3-sulfonamide
Cpd 206 - 4-[(3-chlorophenyl)methyl]-N-(4-cyano-2-fluoro-phenyl)-1H-pyrrole-3-sulfonamide
Cpd 207 - N-(4-cyano-2-fluoro-phenyl)-4-[hydroxy(phenyl)methyl]-1H-pyrrole-3-sulfonamide
Cpd 208 - 4-[(4-chlorophenyl)methyl]-N-(4-cyano-2-fluoro-phenyl)-1H-pyrrole-3-sulfonamide
Cpd 209 - N-(4-cyano-2-fluoro-phenyl)-4-[(4-methoxyphenyl)methyl]-1H-pyrrole-3-sulfonamide
Cpd 210 - N-(4-cyano-2-fluoro-phenyl)-4-[(3-methoxyphenyl)methyl]-1H-pyrrole-3-sulfonamide
Cpd 211 - N-(4-cyano-2-fluoro-phenyl)-4-[(2-methoxyphenyl)methyl]-1H-pyrrole-3-sulfonamide
Cpd 212 - N-(4-cyano-2-fluoro-phenyl)-4-(cyclopentanecarbonyl)-1H-pyrrole-3-sulfonamide
Cpd 213 - 5-cyclopropyl-N-(2,4,5-trifluorophenyl)-1H-pyrrole-3-sulfonamide
Cpd 214 - 5-(4-chloro-2-pyridyl)-N-[2,5-difluoro-4-(trifluoromethyl)phenyl]-1H-pyrrole-3-sulfonamide
Cpd 215 - 5-(6-chloro-2-pyridyl)-N-[2,5-difluoro-4-(trifluoromethyl)phenyl]-1H-pyrrole-3-sulfonamide
Cpd 216 - N-(5-ethynyl-3-fluoro-2-pyridyl)-5-phenyl-1H-pyrrole-3-sulfonamide
Cpd 217 - N-(4-cyano-2-fluoro-phenyl)-4-(4-fluorophenyl)-1H-pyrrole-3-sulfonamide
Cpd 218 - 4-(4-chlorophenyl)-N-(4-cyano-2-fluoro-phenyl)-1H-pyrrole-3-sulfonamide
Cpd 219 - N-(4-cyano-2-fluoro-phenyl)-4-(p-tolyl)-1H-pyrrole-3-sulfonamide
Cpd 220 - N-(4-cyano-2-fluoro-phenyl)-4-(3-methoxyphenyl)-1H-pyrrole-3-sulfonamide
Cpd 221 - N-(4-cyano-2-fluoro-phenyl)-4-(m-tolyl)-1H-pyrrole-3-sulfonamide
Cpd 222 - 4-(2-chlorophenyl)-N-(4-cyano-2-fluoro-phenyl)-1H-pyrrole-3-sulfonamide
Cpd 223 - N-(4-cyano-2-fluoro-phenyl)-4-(2-methoxyphenyl)-1H-pyrrole-3-sulfonamide
Cpd 224 - N-[2,5-difluoro-4-(trifluoromethyl)phenyl]-5-(4-fluoro-2-methoxy-3-pyridyl)-1H-pyrrole-3-sulfonamide
Cpd 225 - N-(4-cyano-2-fluoro-phenyl)-4-(1-phenylethyl)-1H-pyrrole-3-sulfonamide
Cpd 226 - N-[2,5-difluoro-4-(trifluoromethyl)phenyl]-5-(4-fluoro-3-pyridyl)-1H-pyrrole-3-sulfonamide
Cpd 227 - N-(4-chloro-2-fluoro-phenyl)-5-(4-fluoro-2-methoxy-3-pyridyl)-1H-pyrrole-3-sulfonamide
Cpd 228 - 4-(3-chlorophenyl)-N-(4-cyano-2-fluoro-phenyl)-1H-pyrrole-3-sulfonamide
Cpd 229 - N-(4-cyano-2-fluoro-phenyl)-4-(2-pyridyl)-1H-pyrrole-3-sulfonamide
Cpd 230 - N-(4-cyano-2-fluoro-phenyl)-4-(3-fluorophenyl)-1H-pyrrole-3-sulfonamide
Cpd 231 - N-(4-cyano-2-fluoro-phenyl)-4-(2-fluorophenyl)-1H-pyrrole-3-sulfonamide
Cpd 232 - N-[4-(difluoromethoxy)-2,5-difluoro-phenyl]-5-phenyl-1H-pyrrole-3-sulfonamide
Cpd 233 - N-[6-(difluoromethoxy)-3-pyridyl]-5-phenyl-1H-pyrrole-3-sulfonamide
Cpd 234 - N-[2,5-difluoro-4-(trifluoromethyl)phenyl]-5-[2-fluoro-5-(hydroxymethyl)phenyl]-1H-pyrrole-3-sulfonamide
Cpd 235 - N-[2,5-difluoro-4-(trifluoromethyl)phenyl]-5-[2-fluoro-5-(methoxymethyl)phenyl]-1H-pyrrole-3-sulfonamide
Cpd 236 - N-(4-cyano-2-fluoro-phenyl)-5-cyclopropyl-1H-pyrrole-3-sulfonamide
Cpd 237 - N-(4-ethoxy-2,5-difluoro-phenyl)-5-(2-pyridyl)-1H-pyrrole-3-sulfonamide
Cpd 238 - N-(4-cyano-2-fluoro-phenyl)-4-(cyclopenten-1-yl)-1H-pyrrole-3-sulfonamide
Cpd 239 - N-[4-(difluoromethoxy)-2-fluoro-phenyl]-5-(2-pyridyl)-1H-pyrrole-3-sulfonamide
Cpd 240 - N-(4-cyano-2-fluoro-phenyl)-5-(2-ethoxyphenyl)-1H-pyrrole-3-sulfonamide
Cpd 241 - N-(2,5-difluoro-4-isopropoxy-phenyl)-5-(2-pyridyl)-1H-pyrrole-3-sulfonamide
Cpd 242 - N-(4-cyano-2-fluoro-phenyl)-4-cyclopentyl-1H-pyrrole-3-sulfonamide
Cpd 243 - N-[2,5-difluoro-4-(trifluoromethyl)phenyl]-5-[1-(2,2,2-trifluoroethyl)imidazol-2-yl]-1H-pyrrole-3-sulfonamide
Cpd 244 - N-(4-cyano-2-fluoro-phenyl)-5-[2-(difluoromethoxy)phenyl]-1H-pyrrole-3-sulfonamide
Cpd 245 - N-[2,5-difluoro-4-(trifluoromethyl)phenyl]-5-(2-oxopyrrolidin-1-yl)-1H-pyrrole-3-sulfonamide
Cpd 246 - N-[2,5-difluoro-4-(trifluoromethoxy)phenyl]-5-(2-pyridyl)-1H-pyrrole-3-sulfonamide
Cpd 247 - N-[2,5-difluoro-4-(trifluoromethyl)phenyl]-5-(2-fluoro-4-methoxy-3-pyridyl)-1H-pyrrole-3-sulfonamide
Cpd 248 - N-[2,5-difluoro-4-(trifluoromethyl)phenyl]-5-(1-ethylimidazol-2-yl)-1H-pyrrole-3-sulfonamide
Cpd 249 - N-(4-cyano-2-fluoro-phenyl)-5-(3-fluoro-2-methyl-phenyl)-1H-pyrrole-3-sulfonamide
Cpd 250 - 5-(2-chloro-4-methyl-phenyl)-N-(4-cyano-2-fluoro-phenyl)-1H-pyrrole-3-sulfonamide
Cpd 251 - N-(4-cyano-2-fluoro-phenyl)-5-(4-methoxy-2-methyl-phenyl)-1H-pyrrole-3-sulfonamide
Cpd 252 - N-(4-cyano-2-fluoro-phenyl)-5-(2,3-dimethylphenyl)-1H-pyrrole-3-sulfonamide
Cpd 253 - 5-(3-chloro-2-methyl-phenyl)-N-(4-cyano-2-fluoro-phenyl)-1H-pyrrole-3-sulfonamide
Cpd 254 - 5-(2-chloro-5-methyl-phenyl)-N-(4-cyano-2-fluoro-phenyl)-1H-pyrrole-3-sulfonamide
Cpd 255 - N-(4-cyano-2-fluoro-phenyl)-5-(4-fluoro-2-methyl-phenyl)-1H-pyrrole-3-sulfonamide
Cpd 256 - 5-(2-chloro-5-fluoro-phenyl)-N-(4-cyano-2-fluoro-phenyl)-1H-pyrrole-3-sulfonamide
Cpd 257 - 5-(2-chloro-5-methoxy-phenyl)-N-(4-cyano-2-fluoro-phenyl)-1H-pyrrole-3-sulfonamide
Cpd 258 - 5-(2-chloro-4-fluoro-phenyl)-N-(4-cyano-2-fluoro-phenyl)-1H-pyrrole-3-sulfonamide
Cpd 259 - N-(4-cyano-2-fluoro-phenyl)-5-(4-fluoro-3-methoxy-phenyl)-1H-pyrrole-3-sulfonamide
Cpd 260 - N-(4-cyano-2-fluoro-phenyl)-5-(2,5-dichlorophenyl)-1H-pyrrole-3-sulfonamide
Cpd 261 - N-(4-cyano-2-fluoro-phenyl)-5-(2,4-dichlorophenyl)-1H-pyrrole-3-sulfonamide
Cpd 262 - 5-(4-chloro-2-methyl-phenyl)-N-(4-cyano-2-fluoro-phenyl)-1H-pyrrole-3-sulfonamide
Cpd 263 - 5-(2-chloro-3-methyl-phenyl)-N-(4-cyano-2-fluoro-phenyl)-1H-pyrrole-3-sulfonamide
Cpd 264 - N-(4-cyano-2-fluoro-phenyl)-5-(3-fluoro-4-methyl-phenyl)-1H-pyrrole-3-sulfonamide
Cpd 265 - 5-(2-chloro-6-methoxy-phenyl)-N-(4-cyano-2-fluoro-phenyl)-1H-pyrrole-3-sulfonamide
Cpd 266 - 5-(2-chloro-3-methoxy-phenyl)-N-(4-cyano-2-fluoro-phenyl)-1H-pyrrole-3-sulfonamide
Cpd 267 - 5-(2-chloro-3-fluoro-phenyl)-N-(4-cyano-2-fluoro-phenyl)-1H-pyrrole-3-sulfonamide
Cpd 268 - N-(4-cyano-2-fluoro-phenyl)-5-(5-methoxy-2-methyl-phenyl)-1H-pyrrole-3-sulfonamide
Cpd 269 - N-(4-cyano-2-fluoro-phenyl)-5-(4-fluoro-3-methyl-phenyl)-1H-pyrrole-3-sulfonamide
Cpd 270 - N-(4-cyano-2-fluoro-phenyl)-5-(2,6-dichlorophenyl)-1H-pyrrole-3-sulfonamide
Cpd 271 - 4-benzyl-N-[4-(difluoromethoxy)-2,5-difluoro-phenyl]-1H-pyrrole-3-sulfonamide
Cpd 272 - N-[2,5-difluoro-4-(trifluoromethyl)phenyl]-5-(2-thienyl)-1H-pyrrole-3-sulfonamide
Cpd 273 - N-[2,5-difluoro-4-(trifluoromethyl)phenyl]-5-(3-thienyl)-1H-pyrrole-3-sulfonamide
Cpd 274 - N-[2,5-difluoro-4-(trifluoromethyl)phenyl]-5-(5-methyl-2-thienyl)-1H-pyrrole-3-sulfonamide
Cpd 275 - N-[2,5-difluoro-4-(trifluoromethyl)phenyl]-5-(2-furyl)-1H-pyrrole-3-sulfonamide
Cpd 276 - N-[2,5-difluoro-4-(trifluoromethyl)phenyl]-5-(3-furyl)-1H-pyrrole-3-sulfonamide
Cpd 277 - N-[2,5-difluoro-4-(trifluoromethyl)phenyl]-5-(3-methylimidazol-4-yl)-1H-pyrrole-3-sulfonamide
Cpd 278 - N-[2,5-difluoro-4-(trifluoromethyl)phenyl]-5-isothiazol-3-yl-1H-pyrrole-3-sulfonamide
Cpd 279 - N-[2,5-difluoro-4-(trifluoromethyl)phenyl]-5-(6-methyl-2-pyridyl)-1H-pyrrole-3-sulfonamide
Cpd 280 - N-[2,5-difluoro-4-(trifluoromethyl)phenyl]-5-(5-methyl-2-pyridyl)-1H-pyrrole-3-sulfonamide
Cpd 281 - N-[2,5-difluoro-4-(trifluoromethyl)phenyl]-5-(6-methoxy-2-pyridyl)-1H-pyrrole-3-sulfonamide
Cpd 282 - N-[2,5-difluoro-4-(trifluoromethyl)phenyl]-5-[3-(trifluoromethyl)-2-pyridyl]-1H-pyrrole-3-sulfonamide
Cpd 283 - N-(4-cyano-2-fluoro-phenyl)-5-(3-methoxy-2-methyl-phenyl)-1H-pyrrole-3-sulfonamide
Cpd 284 - 5-(3-chloro-4-fluoro-phenyl)-N-(4-cyano-2-fluoro-phenyl)-1H-pyrrole-3-sulfonamide
Cpd 285 - N-(4-cyano-2-fluoro-phenyl)-5-(5-fluoro-2-methyl-phenyl)-1H-pyrrole-3-sulfonamide
Cpd 286 - N-(4-cyano-2-fluoro-5-methyl-phenyl)-5-(2-pyridyl)-1H-pyrrole-3-sulfonamide
Cpd 287 - 5-(4-chloro-3-fluoro-phenyl)-N-(4-cyano-2-fluoro-phenyl)-1H-pyrrole-3-sulfonamide
Cpd 288 - N-(4-cyano-2-fluoro-phenyl)-5-(3-fluoro-5-methyl-phenyl)-1H-pyrrole-3-sulfonamide
Cpd 289 - 5-(2-chloro-6-fluoro-phenyl)-N-(4-cyano-2-fluoro-phenyl)-1H-pyrrole-3-sulfonamide
Cpd 290 - N-(4-cyano-2-fluoro-phenyl)-5-(3-fluoro-2-methoxy-phenyl)-1H-pyrrole-3-sulfonamide
Cpd 291 - N-(4-cyano-2-fluoro-phenyl)-5-(3,4-difluorophenyl)-1H-pyrrole-3-sulfonamide
Cpd 292 - N-(4-cyano-2-fluoro-phenyl)-5-(2,3-dichlorophenyl)-1H-pyrrole-3-sulfonamide
Cpd 293 - N-[2,5-difluoro-4-(trifluoromethyl)phenyl]-5-(2-fluoro-4-methyl-phenyl)-1H-pyrrole-3-sulfonamide
Cpd 294 - N-[2,5-difluoro-4-(trifluoromethyl)phenyl]-5-(4-methoxyphenyl)-1H-pyrrole-3-sulfonamide
Cpd 295 - N-[2,5-difluoro-4-(trifluoromethyl)phenyl]-5-(p-tolyl)-1H-pyrrole-3-sulfonamide
Cpd 296 - 5-(5-chloro-2-methyl-phenyl)-N-(4-cyano-2-fluoro-phenyl)-1H-pyrrole-3-sulfonamide
Cpd 297 - 5-(4-chlorophenyl)-N-[2,5-difluoro-4-(trifluoromethyl)phenyl]-1H-pyrrole-3-sulfonamide
Cpd 298 - N-[2,5-difluoro-4-(trifluoromethyl)phenyl]-5-(2-methoxyphenyl)-1H-pyrrole-3-sulfonamide
Cpd 299 - N-(4-cyano-2-fluoro-phenyl)-5-(3-fluoro-5-methoxy-phenyl)-1H-pyrrole-3-sulfonamide
Cpd 300 - 5-(2-chloro-4-methoxy-phenyl)-N-(4-cyano-2-fluoro-phenyl)-1H-pyrrole-3-sulfonamide
Cpd 301 - 5-(2,3-difluorophenyl)-N-[2,5-difluoro-4-(trifluoromethyl)phenyl]-1H-pyrrole-3-sulfonamide
Cpd 302 - N-[2,5-difluoro-4-(trifluoromethyl)phenyl]-5-thiazol-4-yl-1H-pyrrole-3-sulfonamide
Cpd 303 - N-[2,5-difluoro-4-(trifluoromethyl)phenyl]-5-(4-methyl-2-pyridyl)-1H-pyrrole-3-sulfonamide
Cpd 304 - N-[2,5-difluoro-4-(trifluoromethyl)phenyl]-5-(5-methoxy-2-pyridyl)-1H-pyrrole-3-sulfonamide
Cpd 305 - N-[2,5-difluoro-4-(trifluoromethyl)phenyl]-5-(4-methoxy-2-pyridyl)-1H-pyrrole-3-sulfonamide
Cpd 306 - N-(4-cyano-2-fluoro-phenyl)-4-(m-tolylmethyl)-1H-pyrrole-3-sulfonamide
Cpd 307 - N-(4-cyano-2-fluoro-phenyl)-4-[[3-(trifluoromethyl)phenyl]methyl]-1H-pyrrole-3-sulfonamide
Cpd 308 - N-(4-cyano-2-fluoro-phenyl)-4-[[3-(trifluoromethoxy)phenyl]methyl]-1H-pyrrole-3-sulfonamide
Cpd 309 - N-(4-cyano-2-fluoro-phenyl)-4-[[3-(difluoromethoxy)phenyl]methyl]-1H-pyrrole-3-sulfonamide
Cpd 310 - N-(4-cyano-2-fluoro-phenyl)-5-[3-(trifluoromethyl)-2-pyridyl]-1H-pyrrole-3-sulfonamide
Cpd 311 - N-[4-(cyclopropoxy)-2,5-difluoro-phenyl]-5-(2-pyridyl)-1H-pyrrole-3-sulfonamide
Cpd 312 - N-(3-chloro-4-cyano-2-fluoro-phenyl)-5-phenyl-1H-pyrrole-3-sulfonamide
Cpd 313 - N-[2-fluoro-4-(trifluoromethyl)phenyl]-5-phenyl-1H-pyrrole-3-sulfonamide
Cpd 314 - N-(4-cyano-2-fluoro-phenyl)-5-(5-fluoro-2-methoxy-phenyl)-1H-pyrrole-3-sulfonamide
Cpd 315 - N-[2,5-difluoro-4-(trifluoromethyl)phenyl]-5-(2-fluoro-5-methyl-phenyl)-1H-pyrrole-3-sulfonamide
Cpd 316 - 5-(3-chloro-5-fluoro-phenyl)-N-(4-cyano-2-fluoro-phenyl)-1H-pyrrole-3-sulfonamide
Cpd 317 - N-(4-cyano-2,3-difluoro-phenyl)-5-phenyl-1H-pyrrole-3-sulfonamide
Cpd 318 - 5-(3-cyanophenyl)-N-[2,5-difluoro-4-(trifluoromethyl)phenyl]-1H-pyrrole-3-sulfonamide
Cpd 319 - N-[2,5-difluoro-4-(trifluoromethyl)phenyl]-5-(2-fluoro-3-methyl-phenyl)-1H-pyrrole-3-sulfonamide
Cpd 320 - 5-(5-chloro-2-fluoro-phenyl)-N-[2,5-difluoro-4-(trifluoromethyl)phenyl]-1H-pyrrole-3-sulfonamide
Cpd 321 - N-[2,5-difluoro-4-(trifluoromethyl)phenyl]-5-(2-fluoro-6-methyl-phenyl)-1H-pyrrole-3-sulfonamide
Cpd 322 - N-(4-cyano-2,3-difluoro-phenyl)-5-(2-pyridyl)-1H-pyrrole-3-sulfonamide
Cpd 323 - N-[2,5-difluoro-4-(trifluoromethyl)phenyl]-5-(3-methoxyphenyl)-1H-pyrrole-3-sulfonamide
Cpd 324 - N-[2,5-difluoro-4-(trifluoromethyl)phenyl]-5-(2-fluoro-4-methoxy-phenyl)-1H-pyrrole-3-sulfonamide
Cpd 325 - N-[2,5-difluoro-4-(trifluoromethyl)phenyl]-5-(2-fluoro-3-methoxy-phenyl)-1H-pyrrole-3-sulfonamide
Cpd 326 - N-(4-cyano-2-fluoro-5-methyl-phenyl)-5-phenyl-1H-pyrrole-3-sulfonamide
Cpd 327 - 5-(3-chloro-2-fluoro-phenyl)-N-[2,5-difluoro-4-(trifluoromethyl)phenyl]-1H-pyrrole-3-sulfonamide
Cpd 328 - N-[2,6-difluoro-4-(trifluoromethyl)phenyl]-5-phenyl-1H-pyrrole-3-sulfonamide
Cpd 329 - N-(4-cyano-2-fluoro-phenyl)-5-(2,4-dimethylphenyl)-1H-pyrrole-3-sulfonamide
Cpd 330 - 5-(4-cyanophenyl)-N-[2,5-difluoro-4-(trifluoromethyl)phenyl]-1H-pyrrole-3-sulfonamide
Cpd 331 - N-(4-cyano-2-fluoro-phenyl)-5-(2,5-dimethylphenyl)-1H-pyrrole-3-sulfonamide
Cpd 332 - N-[2,3-difluoro-4-(trifluoromethyl)phenyl]-5-(2-pyridyl)-1H-pyrrole-3-sulfonamide
Cpd 333 - N-(5-chloro-4-cyano-2-fluoro-phenyl)-5-phenyl-1H-pyrrole-3-sulfonamide
Cpd 334 - N-[2,3-difluoro-4-(trifluoromethyl)phenyl]-5-phenyl-1H-pyrrole-3-sulfonamide
Cpd 335 - N-[3-chloro-2-fluoro-4-(trifluoromethyl)phenyl]-5-(2-pyridyl)-1H-pyrrole-3-sulfonamide
Cpd 336 - N-[2-fluoro-5-methyl-4-(trifluoromethyl)phenyl]-5-(2-pyridyl)-1H-pyrrole-3-sulfonamide
Cpd 337 - N-[2,5-difluoro-4-(trifluoromethyl)phenyl]-5-(m-tolyl)-1H-pyrrole-3-sulfonamide
Cpd 338 - N-[2-fluoro-5-methyl-4-(trifluoromethyl)phenyl]-5-phenyl-1H-pyrrole-3-sulfonamide
Cpd 339 - N-[2,5-difluoro-4-(trifluoromethyl)phenyl]-5-thiazol-2-yl-1H-pyrrole-3-sulfonamide
Cpd 340 - N-(4-cyano-2-fluoro-phenyl)-4-[[3-(cyclopropoxy)phenyl]methyl]-1H-pyrrole-3-sulfonamide
Cpd 341 - N-(4-cyano-2-fluoro-phenyl)-4-[(3-isopropoxyphenyl)methyl]-1H-pyrrole-3-sulfonamide
Cpd 342 - N-(4-cyano-2-fluoro-phenyl)-4-[[3-(cyclopropylmethoxy)phenyl]methyl]-1H-pyrrole-3-sulfonamide
Cpd 343 - N-(4-cyano-2-fluoro-phenyl)-4-(2-thienylmethyl)-1H-pyrrole-3-sulfonamide
Cpd 344 - N-[2,5-difluoro-4-(trifluoromethyl)phenyl]-5-[6-(trifluoromethyl)-2-pyridyl]-1H-pyrrole-3-sulfonamide
Cpd 345 - 5-(5-bromo-2-pyridyl)-N-[2,5-difluoro-4-(trifluoromethyl)phenyl]-1H-pyrrole-3-sulfonamide
Cpd 346 - 5-(3-bromo-2-pyridyl)-N-[2,5-difluoro-4-(trifluoromethyl)phenyl]-1H-pyrrole-3-sulfonamide
Cpd 347 - N-(4-cyano-2-fluoro-phenyl)-5-(5-fluoro-2-pyridyl)-1H-pyrrole-3-sulfonamide
Cpd 348 - N-(4-cyano-2-fluoro-phenyl)-5-(3-methyl-2-pyridyl)-1H-pyrrole-3-sulfonamide
Cpd 349 - 5-(3-chloro-2-pyridyl)-N-(4-cyano-2-fluoro-phenyl)-1H-pyrrole-3-sulfonamide
Cpd 350 - 5-(3-bromo-2-pyridyl)-N-(4-cyano-2-fluoro-phenyl)-1H-pyrrole-3-sulfonamide
Cpd 351 - 5-(2-chlorophenyl)-N-[2,5-difluoro-4-(trifluoromethyl)phenyl]-1H-pyrrole-3-sulfonamide
Cpd 352 - N-(4-cyano-2-fluoro-phenyl)-5-(4-fluoro-2-methoxy-phenyl)-1H-pyrrole-3-sulfonamide
Cpd 353 - N-(4-cyano-2,5-difluoro-phenyl)-5-(2-pyridyl)-1H-pyrrole-3-sulfonamide
Cpd 354 - N-(4-cyano-2,6-difluoro-phenyl)-5-(2-pyridyl)-1H-pyrrole-3-sulfonamide
Cpd 355 - 5-(2-cyanophenyl)-N-[2,5-difluoro-4-(trifluoromethyl)phenyl]-1H-pyrrole-3-sulfonamide
Cpd 356 - 5-(3-chlorophenyl)-N-[2,5-difluoro-4-(trifluoromethyl)phenyl]-1H-pyrrole-3-sulfonamide
Cpd 357 - N-(5-chloro-4-cyano-2-fluoro-phenyl)-5-(2-pyridyl)-1H-pyrrole-3-sulfonamide
Cpd 358 - N-(4-cyano-2-fluoro-phenyl)-5-(cyclohexen-1-yl)-1H-pyrrole-3-sulfonamide
Cpd 359 - N-(4-bromo-2,5-difluoro-phenyl)-4-(3-fluorophenyl)-1H-pyrrole-3-sulfonamide
Cpd 360 - N-[4-(difluoromethoxy)-2,5-difluoro-phenyl]-4-(3-fluorophenyl)-1H-pyrrole-3-sulfonamide
Cpd 361 - N-[2,5-difluoro-4-(trifluoromethyl)phenyl]-5-[2-(trifluoromethyl)phenyl]-1H-pyrrole-3-sulfonamide
Cpd 362 - 5-(4-chloro-2-fluoro-phenyl)-N-[2,5-difluoro-4-(trifluoromethyl)phenyl]-1H-pyrrole-3-sulfonamide
Cpd 363 - 5-(2-chloro-6-fluoro-phenyl)-N-[2,5-difluoro-4-(trifluoromethyl)phenyl]-1H-pyrrole-3-sulfonamide
Cpd 364 - N-[2,5-difluoro-4-(trifluoromethyl)phenyl]-4-(3-fluorophenyl)-1H-pyrrole-3-sulfonamide
Cpd 365 - N-[2,5-difluoro-4-(trifluoromethyl)phenyl]-5-[3-(trifluoromethyl)phenyl]-1H-pyrrole-3-sulfonamide
Cpd 366 - 5-(2,4-difluorophenyl)-N-[2,5-difluoro-4-(trifluoromethyl)phenyl]-1H-pyrrole-3-sulfonamide
Cpd 367 - N-(4-cyano-2-fluoro-phenyl)-4-[(3-methylsulfanylphenyl)methyl]-1H-pyrrole-3-sulfonamide
Cpd 368 - N-(4-cyano-2-fluoro-phenyl)-4-[[3-(methoxymethyl)phenyl]methyl]-1H-pyrrole-3-sulfonamide
Cpd 369 - N-[2,5-difluoro-4-(trifluoromethyl)phenyl]-5-[5-(trifluoromethyl)-2-pyridyl]-1H-pyrrole-3-sulfonamide
Cpd 370 - N-[2,5-difluoro-4-(trifluoromethyl)phenyl]-5-[4-(trifluoromethyl)-2-pyridyl]-1H-pyrrole-3-sulfonamide
Cpd 371 - 5-(6-bromo-2-pyridyl)-N-[2,5-difluoro-4-(trifluoromethyl)phenyl]-1H-pyrrole-3-sulfonamide
Cpd 372 - 5-(6-chloro-2-pyridyl)-N-(4-cyano-2-fluoro-phenyl)-1H-pyrrole-3-sulfonamide
Cpd 373 - N-(4-cyano-2-fluoro-phenyl)-5-[6-(trifluoromethyl)-2-pyridyl]-1H-pyrrole-3-sulfonamide
Cpd 374 - 5-(6-bromo-2-pyridyl)-N-(4-cyano-2-fluoro-phenyl)-1H-pyrrole-3-sulfonamide
Cpd 375 - 5-phenyl-N-[2-(trifluoromethyl)thiazol-5-yl]-1H-pyrrole-3-sulfonamide
Cpd 378 - N-[5-chloro-2-fluoro-4-(trifluoromethyl)phenyl]-5-phenyl-1H-pyrrole-3-sulfonamide
Cpd 379 - N-[5-chloro-2-fluoro-4-(trifluoromethyl)phenyl]-5-pyridin-2-yl-1H-pyrrole-3-sulfonamide
Cpd 380 - N-(4-cyano-2-fluorophenyl)-5-quinolin-8-yl-1H-pyrrole-3-sulfonamide
Cpd 381 - N-(4-cyano-2-fluorophenyl)-4-naphthalen-1-yl-1H-pyrrole-3-sulfonamide
Cpd 382 - N-(4-cyano-2-fluorophenyl)-5-naphthalen-1-yl-1H-pyrrole-3-sulfonamide
Cpd 383 - N-[2-fluoro-3-methyl-4-(trifluoromethyl)phenyl]-5-pyridin-2-yl-1H-pyrrole-3-sulfonamide
Cpd 384 - N-(4-cyano-2-fluorophenyl)-4-[(2-methylphenyl)methyl]-1H-pyrrole-3-sulfonamide
Cpd 385 - N-(4-cyano-2-fluorophenyl)-4-[(4-methylphenyl)methyl]-1H-pyrrole-3-sulfonamide
Cpd 386 - N-(4-cyano-2-fluorophenyl)-4-[[2-(trifluoromethyl)phenyl]methyl]-1H-pyrrole-3-sulfonamide
Cpd 387 - N-(4-cyano-2-fluorophenyl)-4-[[4-(trifluoromethyl)phenyl]methyl]-1H-pyrrole-3-sulfonamide
Cpd 388 - N-(4-cyano-2-fluorophenyl)-4-[[2-(trifluoromethoxy)phenyl]methyl]-1H-pyrrole-3-sulfonamide
Cpd 389 - N-(4-cyano-2-fluorophenyl)-4-[[4-(trifluoromethoxy)phenyl]methyl]-1H-pyrrole-3-sulfonamide
Cpd 390 - N-(4-cyano-2-fluorophenyl)-4-[[3-(dimethylamino)phenyl]methyl]-1H-pyrrole-3-sulfonamide
Cpd 391 - 4-[(3-bromophenyl)methyl]-N-(4-cyano-2-fluorophenyl)-1H-pyrrole-3-sulfonamide
Cpd 392 - N-(4-cyano-2-fluorophenyl)-4-(thiophen-3-ylmethyl)-1H-pyrrole-3-sulfonamide
Cpd 393 - 5-(2,5-difluorophenyl)-N-[2,5-difluoro-4-(trifluoromethyl)phenyl]-1H-pyrrole-3-sulfonamide
Cpd 394 - 5-(3-chloro-2-methoxyphenyl)-N-(4-cyano-2-fluorophenyl)-1H-pyrrole-3-sulfonamide
Cpd 395 - 4-(3-tert-butylphenyl)-N-(4-cyano-2-fluorophenyl)-1H-pyrrole-3-sulfonamide
Cpd 396 - N-(4-cyano-2-fluorophenyl)-5-isoquinolin-1-yl-1H-pyrrole-3-sulfonamide
Cpd 397 - N-(4-cyano-2-fluorophenyl)-5-(2-cyclopropyloxyphenyl)-1H-pyrrole-3-sulfonamide
Cpd 398 - N-(4-cyano-2-fluorophenyl)-4-thiophen-2-yl-1H-pyrrole-3-sulfonamide
Cpd 399 - N-[4-chloro-5-(difluoromethoxy)-2-fluorophenyl]-5-phenyl-1H-pyrrole-3-sulfonamide
Cpd 400 - N-(4-cyano-2-fluorophenyl)-4-thiophen-3-yl-1H-pyrrole-3-sulfonamide
Cpd 401 - N-(4-cyano-2-fluorophenyl)-4-(5-methylthiophen-3-yl)-1H-pyrrole-3-sulfonamide
Cpd 402 - N-(4-cyano-2-fluorophenyl)-4-(3-cyclopropylphenyl)-1H-pyrrole-3-sulfonamide
Cpd 403 - N-[4-(difluoromethoxy)-2,5-difluorophenyl]-5-(1,3-thiazol-2-yl)-1H-pyrrole-3-sulfonamide
Cpd 404 - N-[4-(difluoromethoxy)-2,5-difluorophenyl]-5-(3-fluoropyridin-2-yl)-1H-pyrrole-3-sulfonamide
Cpd 405 - N-(4-bromo-2,5-difluorophenyl)-5-(1,3-thiazol-2-yl)-1H-pyrrole-3-sulfonamide
Cpd 406 - N-(4-bromo-2,5-difluorophenyl)-5-(3-fluoropyridin-2-yl)-1H-pyrrole-3-sulfonamide
Cpd 407 - 4-benzyl-N-(4-cyano-2,5-difluorophenyl)-1H-pyrrole-3-sulfonamide
Cpd 408 - N-[5-(difluoromethoxy)-3-fluoropyridin-2-yl]-5-phenyl-1H-pyrrole-3-sulfonamide
Cpd 409 - N-[4-(difluoromethoxy)-2,5-difluorophenyl]-5-(2-fluorophenyl)-1H-pyrrole-3-sulfonamide
Cpd 410 - 5-(5-chloro-2-fluorophenyl)-N-[4-(difluoromethoxy)-2,5-difluorophenyl]-1H-pyrrole-3-sulfonamide
Cpd 411 - N-[4-(difluoromethoxy)-2,5-difluorophenyl]-5-(4-fluorophenyl)-1H-pyrrole-3-sulfonamide
Cpd 412 - N-[4-(difluoromethoxy)-2,5-difluorophenyl]-5-(2,4-difluorophenyl)-1H-pyrrole-3-sulfonamide
Cpd 413 - N-(4-bromo-2,5-difluorophenyl)-5-(2-fluorophenyl)-1H-pyrrole-3-sulfonamide
Cpd 414 - N-(4-bromo-2,5-difluorophenyl)-5-(5-chloro-2-fluorophenyl)-1H-pyrrole-3-sulfonamide
Cpd 415 - 4-benzyl-N-[4-(cyanomethoxy)-2,5-difluorophenyl]-1H-pyrrole-3-sulfonamide
Cpd 416 - N-(4-cyano-2,5-difluorophenyl)-4-(thiophen-2-ylmethyl)-1H-pyrrole-3-sulfonamide
Cpd 417 - N-(4-bromo-2,5-difluorophenyl)-4-(thiophen-2-ylmethyl)-1H-pyrrole-3-sulfonamide
Cpd 418 - N-[2,5-difluoro-4-(trifluoromethyl)phenyl]-4-(thiophen-2-ylmethyl)-1H-pyrrole-3-sulfonamide
Cpd 419 - N-[4-chloro-5-(difluoromethoxy)-2-fluorophenyl]-5-pyridin-2-yl-1H-pyrrole-3-sulfonamide
Cpd 420 - N-(4-cyano-2-fluorophenyl)-4-(5-methylthiophen-2-yl)-1H-pyrrole-3-sulfonamide
Cpd 421 - N-(4-cyano-2-fluorophenyl)-5-cyclohexyl-1H-pyrrole-3-sulfonamide
Cpd 422 - N-(6-chloro-5-fluoro-2-methoxypyridin-3-yl)-5-phenyl-1H-pyrrole-3-sulfonamide
Cpd 423 - 5-cyclohexyl-N-[2,5-difluoro-4-(trifluoromethyl)phenyl]-1H-pyrrole-3-sulfonamide
Cpd 424 - N-[2,5-difluoro-4-(trifluoromethyl)phenyl]-5-(oxolan-3-yl)-1H-pyrrole-3-sulfonamide
Cpd 425 - N-[4-chloro-5-(difluoromethoxy)-2-fluorophenyl]-4-(3-fluorophenyl)-1H-pyrrole-3-sulfonamide
Cpd 426 - 4-benzyl-N-[4-chloro-5-(difluoromethoxy)-2-fluorophenyl]-1H-pyrrole-3-sulfonamide
Cpd 427 - 4-benzyl-N-(6-chloro-5-fluoro-2-methoxypyridin-3-yl)-1H-pyrrole-3-sulfonamide
Cpd 428 - N-(6-chloro-5-fluoro-2-methoxypyridin-3-yl)-4-(3-fluorophenyl)-1H-pyrrole-3-sulfonamide
Cpd 429 - N-[5-(difluoromethoxy)-3-methoxypyridin-2-yl]-5-phenyl-1H-pyrrole-3-sulfonamide
Cpd 430 - 5-cyclobutyl-N-[2,5-difluoro-4-(trifluoromethyl)phenyl]-1H-pyrrole-3-sulfonamide
Cpd 431 - 4-benzyl-N-(5-bromo-3-methoxypyrazin-2-yl)-1H-pyrrole-3-sulfonamide
Cpd 432 - 4-benzyl-N-[5-(2,2-difluoroethoxy)-3-fluoropyridin-2-yl]-1H-pyrrole-3-sulfonamide
Cpd 433 - 4-benzyl-N-[5-(difluoromethoxy)-3-methoxypyridin-2-yl]-1H-pyrrole-3-sulfonamide
Cpd 434 - N-[5-(2,2-difluoroethoxy)-3-fluoropyridin-2-yl]-5-phenyl-1H-pyrrole-3-sulfonamide
Cpd 435 - N-(5-bromo-3-methoxypyrazin-2-yl)-5-phenyl-1H-pyrrole-3-sulfonamide
Cpd 436 - N-[6-(2,2-difluoroethoxy)-5-fluoro-2-methoxypyridin-3-yl]-5-phenyl-1H-pyrrole-3-sulfonamide
Cpd 437 - N-(4-cyano-2-fluorophenyl)-4-pyridin-3-yl-1H-pyrrole-3-sulfonamide
Cpd 438 - N-[5-(2,2-difluoroethoxy)-3-fluoropyridin-2-yl]-4-(3-fluorophenyl)-1H-pyrrole-3-sulfonamide
Cpd 439 - 4-benzyl-N-(5-chloro-4-cyano-2-fluorophenyl)-1H-pyrrole-3-sulfonamide
Cpd 440 - 4-benzyl-N-(4-cyano-2-fluoro-5-methylphenyl)-1H-pyrrole-3-sulfonamide
Cpd 441 - N-(4-cyano-2-fluorophenyl)-5-(furan-3-yl)-1H-pyrrole-3-sulfonamide
Cpd 442 - 5-(1-benzofuran-7-yl)-N-(4-cyano-2-fluorophenyl)-1H-pyrrole-3-sulfonamide
Cpd 443 - N-(4-cyano-2-fluorophenyl)-5-(2,3-dihydro-1-benzofuran-7-yl)-1H-pyrrole-3-sulfonamide
Cpd 444 - 4-benzyl-N-[6-(2,2-difluoroethoxy)-5-fluoro-2-methoxypyridin-3-yl]-1H-pyrrole-3-sulfonamide
Cpd 445 - N-(4-cyano-2-fluorophenyl)-5-(furan-2-yl)-1H-pyrrole-3-sulfonamide
Cpd 446 - N-(4-bromo-2,5-difluorophenyl)-5-(4-fluorophenyl)-1H-pyrrole-3-sulfonamide
Cpd 447 - N-(4-bromo-2,5-difluorophenyl)-5-(2,4-difluorophenyl)-1H-pyrrole-3-sulfonamide
Cpd 448 - N-[4-(cyanomethoxy)-2,5-difluorophenyl]-5-(2-fluorophenyl)-1H-pyrrole-3-sulfonamide
Cpd 449 - 5-(5-chloro-2-fluorophenyl)-N-[4-(cyanomethoxy)-2,5-difluorophenyl]-1H-pyrrole-3-sulfonamide
Cpd 450 - N-[4-(cyanomethoxy)-2,5-difluorophenyl]-5-(4-fluorophenyl)-1H-pyrrole-3-sulfonamide
Cpd 451 - N-[4-(cyanomethoxy)-2,5-difluorophenyl]-5-(2,4-difluorophenyl)-1H-pyrrole-3-sulfonamide
Cpd 452 - N-[4-(difluoromethoxy)-2,5-difluorophenyl]-4-(thiophen-2-ylmethyl)-1H-pyrrole-3-sulfonamide
Cpd 453 - N-[4-(difluoromethoxy)-2,5-difluorophenyl]-4-[(3-fluorophenyl)methyl]-1H-pyrrole-3-sulfonamide
Cpd 454 - 4-[(3-chlorophenyl)methyl]-N-[4-(difluoromethoxy)-2,5-difluorophenyl]-1H-pyrrole-3-sulfonamide
Cpd 455 - N-[5-chloro-4-(difluoromethoxy)-2-fluorophenyl]-5-phenyl-1H-pyrrole-3-sulfonamide
Cpd 456 - 5-(5-chloro-2,4-difluorophenyl)-N-(4-cyano-2,5-difluorophenyl)-1H-pyrrole-3-sulfonamide
Cpd 457 - N-[2,5-difluoro-4-(trifluoromethyl)phenyl]-5-(5-methylthiophen-3-yl)-1H-pyrrole-3-sulfonamide
Cpd 458 - 5-(5-chlorothiophen-3-yl)-N-[2,5-difluoro-4-(trifluoromethyl)phenyl]-1H-pyrrole-3-sulfonamide
Cpd 459 - 5-(2-chlorophenyl)-N-[4-(cyanomethoxy)-2,5-difluorophenyl]-1H-pyrrole-3-sulfonamide
Cpd 460 - N-[6-(2,2-difluoroethoxy)-5-fluoro-2-methoxypyridin-3-yl]-4-(3-fluorophenyl)-1H-pyrrole-3-sulfonamide
Cpd 461 - N-(4-cyano-2-fluorophenyl)-5-methyl-4-phenyl-1H-pyrrole-3-sulfonamide
Cpd 462 - N-(4-cyano-2,5-difluorophenyl)-5-(2,4,6-trifluorophenyl)-1H-pyrrole-3-sulfonamide
Cpd 463 - N-[4-(2,2-difluoroethoxy)-2,5-difluorophenyl]-5-phenyl-1H-pyrrole-3-sulfonamide
Cpd 464 - N-[5-(cyanomethyl)-3-methoxypyridin-2-yl]-5-(2-fluorophenyl)-1H-pyrrole-3-sulfonamide
Cpd 465 - N-[2,5-difluoro-4-(trifluoromethyl)phenyl]-5-quinolin-8-yl-1H-pyrrole-3-sulfonamide
Cpd 466 - N-[4-(cyanomethoxy)-2,5-difluorophenyl]-4-(3-fluorophenyl)-1H-pyrrole-3-sulfonamide
Cpd 467 - N-[4-(difluoromethoxy)-2-fluoro-5-methylphenyl]-5-phenyl-1H-pyrrole-3-sulfonamide
Cpd 468 - 4-benzyl-N-[4-(2,2-difluoroethoxy)-2,5-difluorophenyl]-1H-pyrrole-3-sulfonamide
Cpd 469 - N-(5-bromo-3-methoxypyrazin-2-yl)-4-(3-fluorophenyl)-1H-pyrrole-3-sulfonamide
Cpd 470 - N-(5-chloro-4-cyano-2-fluorophenyl)-5-(5-chloro-2,4-difluorophenyl)-1H-pyrrole-3-sulfonamide
Cpd 471 - 5-(5-chloro-2,4-difluorophenyl)-N-(4-cyano-2-fluoro-5-methylphenyl)-1H-pyrrole-3-sulfonamide
Cpd 472 - N-[5-(cyanomethoxy)-3-fluoropyridin-2-yl]-5-phenyl-1H-pyrrole-3-sulfonamide
Cpd 473 - N-[4-(2,2-difluoroethoxy)-2,5-difluorophenyl]-5-pyridin-2-yl-1H-pyrrole-3-sulfonamide
Cpd 474 - N-[4-(difluoromethoxy)-2,5-difluorophenyl]-5-(furan-3-yl)-1H-pyrrole-3-sulfonamide
Cpd 475 - N-[4-(difluoromethoxy)-2,5-difluorophenyl]-5-thiophen-2-yl-1H-pyrrole-3-sulfonamide
Cpd 476 - N-[4-(difluoromethoxy)-2,5-difluorophenyl]-5-thiophen-3-yl-1H-pyrrole-3-sulfonamide
Cpd 477 - N-(4-bromo-2,5-difluorophenyl)-5-(furan-3-yl)-1H-pyrrole-3-sulfonamide
Cpd 478 - N-(4-bromo-2,5-difluorophenyl)-5-thiophen-2-yl-1H-pyrrole-3-sulfonamide
Cpd 479 - N-(4-bromo-2,5-difluorophenyl)-5-thiophen-3-yl-1H-pyrrole-3-sulfonamide
Cpd 480 - N-[4-(difluoromethoxy)-2,5-difluorophenyl]-4-[(3-methoxyphenyl)methyl]-1H-pyrrole-3-sulfonamide
Cpd 481 - N-[2,5-difluoro-4-(trifluoromethyl)phenyl]-4-[(3-fluorophenyl)methyl]-1H-pyrrole-3-sulfonamide
Cpd 482 - N-[2,5-difluoro-4-(trifluoromethyl)phenyl]-4-[(3-methoxyphenyl)methyl]-1H-pyrrole-3-sulfonamide
Cpd 483 - N-[4-(difluoromethoxy)-2,5-difluorophenyl]-4-[[3-(difluoromethoxy)phenyl]methyl]-1H-pyrrole-3-sulfonamide
Cpd 484 - N-[4-(difluoromethoxy)-2,5-difluorophenyl]-4-[(2-fluorophenyl)methyl]-1H-pyrrole-3-sulfonamide
Cpd 485 - N-[4-(difluoromethoxy)-2,5-difluorophenyl]-4-[(4-fluorophenyl)methyl]-1H-pyrrole-3-sulfonamide
Cpd 486 - N-(4-cyano-2-fluorophenyl)-4-[(5-methylthiophen-2-yl)methyl]-1H-pyrrole-3-sulfonamide
Cpd 487 - N-(4-cyano-2-fluorophenyl)-5-[4-(trifluoromethyl)phenyl]-1H-pyrrole-3-sulfonamide
Cpd 488 - 5-(5-chloro-2-methoxyphenyl)-N-(4-cyano-2-fluorophenyl)-1H-pyrrole-3-sulfonamide
Cpd 489 - N-(4-cyano-2-fluorophenyl)-5-(2,4,6-trifluorophenyl)-1H-pyrrole-3-sulfonamide
Cpd 490 - 4-[(3-acetylphenyl)methyl]-N-(4-cyano-2-fluorophenyl)-1H-pyrrole-3-sulfonamide
Cpd 491 - N-(4-cyano-2-fluorophenyl)-5-(pyridin-2-ylmethyl)-1H-pyrrole-3-sulfonamide
Cpd 492 - N-(5-chloro-4-cyano-2-fluorophenyl)-5-(2,4,6-trifluorophenyl)-1H-pyrrole-3-sulfonamide
Cpd 493 - N-(4-cyano-2-fluorophenyl)-5-(1-pyridin-2-ylethyl)-1H-pyrrole-3-sulfonamide
Cpd 494 - N-[3,6-difluoro-5-(2-fluoroethoxy)pyridin-2-yl]-5-phenyl-1H-pyrrole-3-sulfonamide
Cpd 495 - N-(4-cyano-2-fluoro-5-methylphenyl)-5-(2,4,6-trifluorophenyl)-1H-pyrrole-3-sulfonamide
Cpd 496 - N-[4-(2,2-difluoroethoxy)-2,5-difluorophenyl]-4-(3-fluorophenyl)-1H-pyrrole-3-sulfonamide
Cpd 497 - N-(4-cyano-2,5-difluorophenyl)-4-(3-fluorophenyl)-1H-pyrrole-3-sulfonamide
Cpd 498 - N-(4-cyano-2-fluorophenyl)-5-(5-fluoro-6-methylpyridin-2-yl)-1H-pyrrole-3-sulfonamide
Cpd 499 - 5-(5-chloropyridin-2-yl)-N-(4-cyano-2-fluorophenyl)-1H-pyrrole-3-sulfonamide
Cpd 500 - N-(4-cyano-2-fluorophenyl)-5-(5-methylpyridin-2-yl)-1H-pyrrole-3-sulfonamide
Cpd 501 - 4-benzyl-N-[3,6-difluoro-5-(2-fluoroethoxy)pyridin-2-yl]-1H-pyrrole-3-sulfonamide
Cpd 502 - N-(4-cyano-2-fluorophenyl)-5-(6-fluoropyridin-2-yl)-1H-pyrrole-3-sulfonamide
Cpd 503 - N-(4-cyano-2-fluorophenyl)-5-(4-methylpyridin-2-yl)-1H-pyrrole-3-sulfonamide
Cpd 504 - N-(4-cyano-2-fluorophenyl)-5-(6-methylpyridin-2-yl)-1H-pyrrole-3-sulfonamide
Cpd 505 - N-[2,5-difluoro-4-(trifluoromethyl)phenyl]-5-(3-oxocyclopenten-1-yl)-1H-pyrrole-3-sulfonamide
Cpd 506 - N-(4-cyano-2-fluorophenyl)-5-[2-(dimethylamino)phenyl]-1H-pyrrole-3-sulfonamide
Cpd 507 - 5-(5-chloro-2,4-difluorophenyl)-N-(4-cyano-2-fluorophenyl)-1H-pyrrole-3-sulfonamide
Cpd 508 - 5-(5-chloro-2-fluorophenyl)-N-(4-cyano-2,5-difluorophenyl)-1H-pyrrole-3-sulfonamide
Cpd 509 - N-(4-cyano-2,5-difluorophenyl)-5-(4-fluorophenyl)-1H-pyrrole-3-sulfonamide
Cpd 510 - N-(4-cyano-2,5-difluorophenyl)-5-(2,4-difluorophenyl)-1H-pyrrole-3-sulfonamide
Cpd 511 - N-(4-cyano-2-fluoro-5-methylphenyl)-5-(2-fluorophenyl)-1H-pyrrole-3-sulfonamide
Cpd 512 - 5-(5-chloro-2-fluorophenyl)-N-(4-cyano-2-fluoro-5-methylphenyl)-1H-pyrrole-3-sulfonamide
Cpd 513 - N-(4-cyano-2-fluoro-5-methylphenyl)-5-(4-fluorophenyl)-1H-pyrrole-3-sulfonamide
Cpd 514 - N-(4-cyano-2-fluoro-5-methylphenyl)-5-(2,4-difluorophenyl)-1H-pyrrole-3-sulfonamide
Cpd 515 - 5-(6-chloropyridin-2-yl)-N-[4-(difluoromethoxy)-2,5-difluorophenyl]-1H-pyrrole-3-sulfonamide
Cpd 516 - 5-(6-bromopyridin-2-yl)-N-[4-(difluoromethoxy)-2,5-difluorophenyl]-1H-pyrrole-3-sulfonamide
Cpd 517 - 5-(1-benzofuran-7-yl)-N-[2,5-difluoro-4-(trifluoromethyl)phenyl]-1H-pyrrole-3-sulfonamide
Cpd 518 - N-[2,5-difluoro-4-(trifluoromethyl)phenyl]-5-(4-methoxythiophen-3-yl)-1H-pyrrole-3-sulfonamide
Cpd 519 - N-[2,5-difluoro-4-(trifluoromethyl)phenyl]-5-(2,3-dihydro-1-benzofuran-7-yl)-1H-pyrrole-3-sulfonamide
Cpd 520 - N-[2,5-difluoro-4-(trifluoromethyl)phenyl]-5-(3-oxocyclopentyl)-1H-pyrrole-3-sulfonamide
Cpd 521 - N-(4-cyano-2-fluorophenyl)-5-[3-(dimethylamino)phenyl]-1H-pyrrole-3-sulfonamide
Cpd 522 - N-(5-chloro-4-cyano-2-fluorophenyl)-5-(2-fluorophenyl)-1H-pyrrole-3-sulfonamide
Cpd 523 - N-(5-chloro-4-cyano-2-fluorophenyl)-5-(5-chloro-2-fluorophenyl)-1H-pyrrole-3-sulfonamide
Cpd 524 - N-(5-chloro-4-cyano-2-fluorophenyl)-5-(4-fluorophenyl)-1H-pyrrole-3-sulfonamide
Cpd 525 - N-(5-chloro-4-cyano-2-fluorophenyl)-5-(2,4-difluorophenyl)-1H-pyrrole-3-sulfonamide
Cpd 526 - 5-(2-chlorophenyl)-N-[4-(difluoromethoxy)-2,5-difluorophenyl]-1H-pyrrole-3-sulfonamide
Cpd 527 - N-(4-bromo-2,5-difluorophenyl)-5-(6-chloropyridin-2-yl)-1H-pyrrole-3-sulfonamide
Cpd 528 - N-(4-bromo-2,5-difluorophenyl)-5-(6-bromopyridin-2-yl)-1H-pyrrole-3-sulfonamide
Cpd 529 - N-(4-cyano-5-ethyl-2-fluorophenyl)-5-phenyl-1H-pyrrole-3-sulfonamide
Cpd 530 - N-[4-(difluoromethoxy)-2,5-difluorophenyl]-5-(2-methoxyphenyl)-1H-pyrrole-3-sulfonamide
Cpd 531 - N-[4-(difluoromethoxy)-2,5-difluorophenyl]-5-(3-fluoro-2-methoxyphenyl)-1H-pyrrole-3-sulfonamide
Cpd 532 - N-(4-bromo-2,5-difluorophenyl)-5-(2-methoxyphenyl)-1H-pyrrole-3-sulfonamide
Cpd 533 - N-(4-bromo-2,5-difluorophenyl)-5-(2-chlorophenyl)-1H-pyrrole-3-sulfonamide
Cpd 534 - N-(4-bromo-2,5-difluorophenyl)-5-quinolin-8-yl-1H-pyrrole-3-sulfonamide
Cpd 535 - N-(4-cyano-2-fluorophenyl)-4-[(3,4-difluorophenyl)methyl]-1H-pyrrole-3-sulfonamide
Cpd 536 - N-(4-cyano-2-fluorophenyl)-4-[(4-fluoro-3-methoxyphenyl)methyl]-1H-pyrrole-3-sulfonamide
Cpd 537 - N-[2,5-difluoro-4-(hydroxymethyl)phenyl]-5-phenyl-1H-pyrrole-3-sulfonamide
Cpd 538 - N-[2,5-difluoro-4-(trifluoromethyl)phenyl]-5-(2-methyl-1,3-thiazol-5-yl)-1H-pyrrole-3-sulfonamide
Cpd 539 - N-[2,5-difluoro-4-(trifluoromethyl)phenyl]-5-(2-methoxythiophen-3-yl)-1H-pyrrole-3-sulfonamide
Cpd 540 - N-[2,5-difluoro-4-(trifluoromethyl)phenyl]-5-(2-methylindazol-7-yl)-1H-pyrrole-3-sulfonamide
Cpd 541 - N-(4-bromo-2,5-difluorophenyl)-5-(3-cyanophenyl)-1H-pyrrole-3-sulfonamide
Cpd 542 - N-(4-cyano-2-fluorophenyl)-5-(2-cyclopropyloxy-3-fluorophenyl)-1H-pyrrole-3-sulfonamide
Cpd 543 - N-(4-cyano-2-fluorophenyl)-5-(3,5-difluoropyridin-2-yl)-1H-pyrrole-3-sulfonamide
Cpd 544 - N-(4-bromo-2,5-difluorophenyl)-5-(3-fluoro-2-methoxyphenyl)-1H-pyrrole-3-sulfonamide
Cpd 545 - 4-[(3-chloro-4-fluorophenyl)methyl]-N-(4-cyano-2-fluorophenyl)-1H-pyrrole-3-sulfonamide
Cpd 546 - N-(4-cyano-2-fluorophenyl)-4-[(3,5-difluorophenyl)methyl]-1H-pyrrole-3-sulfonamide
Cpd 547 - N-(4-cyano-2-fluorophenyl)-4-[(3-fluoro-5-methoxyphenyl)methyl]-1H-pyrrole-3-sulfonamide
Cpd 548 - N-(4-cyano-2,5-difluorophenyl)-4-[[3-(difluoromethoxy)phenyl]methyl]-1H-pyrrole-3-sulfonamide
Cpd 549 - N-(4-cyano-2,5-difluorophenyl)-5-cyclobutyl-1H-pyrrole-3-sulfonamide
Cpd 550 - 5-cyclobutyl-N-[4-(difluoromethoxy)-2,5-difluorophenyl]-1H-pyrrole-3-sulfonamide
Cpd 551 - N-(4-bromo-2,5-difluorophenyl)-5-cyclobutyl-1H-pyrrole-3-sulfonamide
Cpd 552 - N-[4-(difluoromethoxy)-2,5-difluorophenyl]-5-quinolin-8-yl-1H-pyrrole-3-sulfonamide
Cpd 553 - 5-(1-benzofuran-7-yl)-N-[4-(difluoromethoxy)-2,5-difluorophenyl]-1H-pyrrole-3-sulfonamide
Cpd 554 - 5-(5-cyano-2-fluorophenyl)-N-[2,5-difluoro-4-(trifluoromethyl)phenyl]-1H-pyrrole-3-sulfonamide
Cpd 555 - N-[2,5-difluoro-4-(trifluoromethyl)phenyl]-5-(1-methyl-2-oxopyridin-3-yl)-1H-pyrrole-3-sulfonamide
Cpd 556 - 5-(4-chloropyridin-3-yl)-N-[2,5-difluoro-4-(trifluoromethyl)phenyl]-1H-pyrrole-3-sulfonamide
Cpd 557 - 5-(2,4-difluoropyridin-3-yl)-N-[2,5-difluoro-4-(trifluoromethyl)phenyl]-1H-pyrrole-3-sulfonamide
Cpd 558 - 4-[[3-(difluoromethoxy)phenyl]methyl]-N-[2,5-difluoro-4-(trifluoromethyl)phenyl]-1H-pyrrole-3-sulfonamide
Cpd 559 - N-[2,5-difluoro-4-(trifluoromethyl)phenyl]-5-(1,2-thiazol-4-yl)-1H-pyrrole-3-sulfonamide
Cpd 560 - N-[2,5-difluoro-4-(trifluoromethyl)phenyl]-5-[3-(methoxymethyl)pyridin-2-yl]-1H-pyrrole-3-sulfonamide
Cpd 561 - 5-(1-benzofuran-7-yl)-N-(4-bromo-2,5-difluorophenyl)-1H-pyrrole-3-sulfonamide
Cpd 562 - 5-(5-chlorothiophen-3-yl)-N-(4-cyano-2-fluorophenyl)-1H-pyrrole-3-sulfonamide
Cpd 563 - 5-(4-chlorothiophen-2-yl)-N-(4-cyano-2-fluorophenyl)-1H-pyrrole-3-sulfonamide
Cpd 564 - 5-(3-cyano-2-fluorophenyl)-N-[2,5-difluoro-4-(trifluoromethyl)phenyl]-1H-pyrrole-3-sulfonamide
Cpd 565 - N-[2,5-difluoro-4-(trifluoromethyl)phenyl]-5-imidazo[1,2-a]pyridin-8-yl-1H-pyrrole-3-sulfonamide
Cpd 566 - N-[2,5-difluoro-4-(trifluoromethyl)phenyl]-5-pyrazolo[1,5-a]pyridin-7-yl-1H-pyrrole-3-sulfonamide
Cpd 567 - N-[2,5-difluoro-4-(trifluoromethyl)phenyl]-5-pyrrolo[1,2-b]pyridazin-7-yl-1H-pyrrole-3-sulfonamide
Cpd 568 - 4-[(3-chlorophenyl)methyl]-N-[2,5-difluoro-4-(trifluoromethyl)phenyl]-1H-pyrrole-3-sulfonamide
Cpd 569 - 4-[(3-chlorophenyl)methyl]-N-(4-cyano-2,5-difluorophenyl)-1H-pyrrole-3-sulfonamide
Cpd 570 - N-(4-cyano-2-fluorophenyl)-5-(5-fluorothiophen-2-yl)-1H-pyrrole-3-sulfonamide
Cpd 571 - N-[2,5-difluoro-4-(trifluoromethyl)phenyl]-5-(3,3-dimethylcyclobutyl)-1H-pyrrole-3-sulfonamide
Cpd 572 - 5-(1-benzothiophen-7-yl)-N-(4-cyano-2,5-difluorophenyl)-1H-pyrrole-3-sulfonamide
Cpd 573 - N-[2,5-difluoro-4-(trifluoromethyl)phenyl]-5-[3-(fluoromethyl)pyridin-2-yl]-1H-pyrrole-3-sulfonamide
Cpd 574 - 4-[(3-cyanophenyl)methyl]-N-[2,5-difluoro-4-(trifluoromethyl)phenyl]-1H-pyrrole-3-sulfonamide
Cpd 575 - 5-(6-chloropyridin-2-yl)-N-(4-cyano-2,5-difluorophenyl)-1H-pyrrole-3-sulfonamide
Cpd 576 - N-[5-chloro-4-(cyanomethoxy)-2-fluorophenyl]-5-phenyl-1H-pyrrole-3-sulfonamide
Cpd 577 - N-(4-cyano-2-fluorophenyl)-5-(5-fluoro-3-methylpyridin-2-yl)-1H-pyrrole-3-sulfonamide
Cpd 578 - N-[4-(cyanomethoxy)-2,5-difluorophenyl]-5-cyclobutyl-1H-pyrrole-3-sulfonamide
Cpd 579 - N-(1,2-oxazol-3-yl)-5-phenyl-1H-pyrrole-3-sulfonamide
Cpd 580 - 5-[3-(difluoromethyl)pyridin-2-yl]-N-[2,5-difluoro-4-(trifluoromethyl)phenyl]-1H-pyrrole-3-sulfonamide
Cpd 581 - N-(4-cyano-2-fluorophenyl)-5-[3-(difluoromethyl)pyridin-2-yl]-1H-pyrrole-3-sulfonamide
Cpd 582 - N-[2,5-difluoro-4-(trifluoromethyl)phenyl]-5-(3,5-dimethyl-1,2-oxazol-4-yl)-1H-pyrrole-3-sulfonamide
Cpd 583 - N-(1,3-oxazol-2-yl)-5-phenyl-1H-pyrrole-3-sulfonamide
Cpd 584 - N-(4-cyano-2,5-difluorophenyl)-4-[(4-fluorophenyl)methyl]-1H-pyrrole-3-sulfonamide
Cpd 585 - N-(4-cyano-2,5-difluorophenyl)-4-[(3-fluorophenyl)methyl]-1H-pyrrole-3-sulfonamide
Cpd 586 - N-(4-cyano-2,5-difluorophenyl)-4-[(3-cyclopropylphenyl)methyl]-1H-pyrrole-3-sulfonamide
Cpd 587 - N-(4-cyano-2-fluorophenyl)-5-[2-(trifluoromethoxy)phenyl]-1H-pyrrole-3-sulfonamide
Cpd 588 - N-(4-cyano-2,5-difluorophenyl)-5-[2-(trifluoromethoxy)phenyl]-1H-pyrrole-3-sulfonamide
Cpd 589 - N-[2,5-difluoro-4-(trifluoromethyl)phenyl]-5-[2-(trifluoromethoxy)phenyl]-1H-pyrrole-3-sulfonamide
Cpd 590 - 4-[(3-cyclopropylphenyl)methyl]-N-[2,5-difluoro-4-(trifluoromethyl)phenyl]-1H-pyrrole-3-sulfonamide
Cpd 591 - 5-phenyl-N-(1,2-thiazol-4-yl)-1H-pyrrole-3-sulfonamide
Cpd 592 - N-(4-cyano-2-fluorophenyl)-5-[3-(fluoromethyl)pyridin-2-yl]-1H-pyrrole-3-sulfonamide
Cpd 593 - N-(4-fluorothiophen-2-yl)-5-phenyl-1H-pyrrole-3-sulfonamide
Cpd 594 - N-(4-cyano-2-fluorophenyl)-5-(4-fluorothiophen-3-yl)-1H-pyrrole-3-sulfonamide
Cpd 595 - N-[2,5-difluoro-4-(trifluoromethyl)phenyl]-5-(2-methyl-1,3-thiazol-4-yl)-1H-pyrrole-3-sulfonamide
Cpd 596 - 5-(3-chloro-4-fluorophenyl)-N-(4-cyano-2,5-difluorophenyl)-1H-pyrrole-3-sulfonamide
Cpd 597 - N-[2,5-difluoro-4-(trifluoromethyl)phenyl]-5-(1,3-thiazol-5-yl)-1H-pyrrole-3-sulfonamide
Cpd 598 - N-(4-cyano-2,5-difluorophenyl)-5-(1,3-thiazol-2-yl)-1H-pyrrole-3-sulfonamide
Cpd 599 - N-[2,5-difluoro-4-(trifluoromethyl)phenyl]-5-(4-methyl-1,3-thiazol-2-yl)-1H-pyrrole-3-sulfonamide
Cpd 600 - N-(4-cyano-2-fluorophenyl)-5-(1-methyl-2-oxopyridin-3-yl)-1H-pyrrole-3-sulfonamide
Cpd 601 - N-(5-cyano-3-fluorothiophen-2-yl)-5-phenyl-1H-pyrrole-3-sulfonamide
Cpd 602 - N-(4-cyano-2-fluorophenyl)-5-(3-fluorothiophen-2-yl)-1H-pyrrole-3-sulfonamide
Cpd 603 - N-(4-cyano-2-fluorophenyl)-5-(2-oxo-1-propan-2-ylpyridin-3-yl)-1H-pyrrole-3-sulfonamide
Cpd 604 - N-(4-cyano-2-fluorophenyl)-5-[1-(difluoromethyl)-2-oxopyridin-3-yl]-1H-pyrrole-3-sulfonamide
Cpd 605 - 4-[(3-chloro-5-fluorophenyl)methyl]-N-(4-cyano-2,5-difluorophenyl)-1H-pyrrole-3-sulfonamide
Cpd 606 - N-[2,5-difluoro-4-(trifluoromethyl)phenyl]-5-(5-methyl-1,3-thiazol-2-yl)-1H-pyrrole-3-sulfonamide
Cpd 607 - 5-(5-cyano-1,3-thiazol-2-yl)-N-[2,5-difluoro-4-(trifluoromethyl)phenyl]-1H-pyrrole-3-sulfonamide
Cpd 608 - N-(4-cyano-2-fluorophenyl)-4-(2,3-dihydro-1-benzofuran-6-ylmethyl)-1H-pyrrole-3-sulfonamide
Cpd 609 - 4-benzyl-N-(4-cyano-2-fluorophenyl)-5-fluoro-1H-pyrrole-3-sulfonamide
Cpd 610 - N-(4-cyano-2-fluorophenyl)-5-(1,3-thiazol-2-yl)-1H-pyrrole-3-sulfonamide
Cpd 611 - N-[4-(difluoromethoxy)-2,5-difluorophenyl]-5-(1,3,4-oxadiazol-2-yl)-1H-pyrrole-3-sulfonamide
Cpd 612 - N-(4-cyano-2-fluorophenyl)-5-(4-deuteriophenyl)-1H-pyrrole-3-sulfonamide
Cpd 613 - N-[4-(difluoromethoxy)-2,5-difluorophenyl]-5-(1,2,4-thiadiazol-5-yl)-1H-pyrrole-3-sulfonamide
Cpd 614 - N-(4-cyano-2-fluorophenyl)-5-[2-oxo-1-(2,2,2-trifluoroethyl)pyridin-3-yl]-1H-pyrrole-3-sulfonamide
Cpd 615 - 5-(1-benzofuran-7-yl)-N-(4-cyano-2,5-difluorophenyl)-1H-pyrrole-3-sulfonamide
Cpd 616 - N-(4-cyano-2-fluorophenyl)-4-(cyclohexylmethyl)-1H-pyrrole-3-sulfonamide
Cpd 617 - 5-(5-cyano-1,3-thiazol-2-yl)-N-[4-(difluoromethoxy)-2,5-difluorophenyl]-1H-pyrrole-3-sulfonamide
Cpd 618 - N-[2,5-difluoro-4-(trifluoromethyl)phenyl]-5-pyrazin-2-yl-1H-pyrrole-3-sulfonamide
Cpd 619 - N-[2,5-difluoro-4-(trifluoromethyl)phenyl]-5-pyridazin-3-yl-1H-pyrrole-3-sulfonamide
Cpd 620 - N-(4-cyano-2-fluorophenyl)-5-(1-methylbenzimidazol-4-yl)-1H-pyrrole-3-sulfonamide
Cpd 621 - 5-(3,3-difluorocyclopentyl)-N-[2,5-difluoro-4-(trifluoromethyl)phenyl]-1H-pyrrole-3-sulfonamide
Cpd 622 - N-(4-cyano-2-fluorophenyl)-5-(cyclopropylmethyl)-1H-pyrrole-3-sulfonamide
Cpd 623 - N-(4-cyano-2-fluorophenyl)-4-(pyridin-2-ylmethyl)-1H-pyrrole-3-sulfonamide
Cpd 624 - N-(4-cyano-2-fluorophenyl)-4-(pyridin-4-ylmethyl)-1H-pyrrole-3-sulfonamide
Cpd 625 - N-(4-cyano-2-fluorophenyl)-4-(2-phenylethyl)-1H-pyrrole-3-sulfonamide
Cpd 626 - N-[2,5-difluoro-4-(trifluoromethyl)phenyl]-4-fluoro-5-phenyl-1H-pyrrole-3-sulfonamide
Cpd 627 - N-[2,5-difluoro-4-(trifluoromethyl)phenyl]-2-fluoro-5-phenyl-1H-pyrrole-3-sulfonamide
Cpd 628 - N-[4-(difluoromethoxy)-2,5-difluorophenyl]-5-(1,3-thiazol-5-yl)-1H-pyrrole-3-sulfonamide
Cpd 629 - N-[4-(difluoromethoxy)-2,5-difluorophenyl]-5-(1,3-thiazol-4-yl)-1H-pyrrole-3-sulfonamide
Cpd 630 - 5-phenyl-N-[5-(trifluoromethyl)-1,3-thiazol-2-yl]-1H-pyrrole-3-sulfonamide
Cpd 631 - N-(5-cyano-1,3-thiazol-2-yl)-5-phenyl-1H-pyrrole-3-sulfonamide
Cpd 632 - N-[4-(cyanomethoxy)-2,5-difluorophenyl]-4-fluoro-5-phenyl-1H-pyrrole-3-sulfonamide
Cpd 633 - N-[4-(difluoromethoxy)-2,5-difluorophenyl]-5-(4-methoxy-1,3-thiazol-2-yl)-1H-pyrrole-3-sulfonamide
Cpd 634 - N-[4-(difluoromethoxy)-2,5-difluorophenyl]-5-(4-methyl-1,3-thiazol-2-yl)-1H-pyrrole-3-sulfonamide
Cpd 635 - N-[4-(difluoromethoxy)-2,5-difluorophenyl]-5-(1,2-thiazol-3-yl)-1H-pyrrole-3-sulfonamide
Cpd 636 - N-[4-(difluoromethoxy)-2,5-difluorophenyl]-5-(1,3-oxazol-2-yl)-1H-pyrrole-3-sulfonamide
Cpd 637 - N-[4-(difluoromethoxy)-2,5-difluorophenyl]-5-[5-(trifluoromethyl)-1,3-thiazol-2-yl]-1H-pyrrole-3-sulfonamide
Cpd 638 - N-[4-(difluoromethoxy)-2,5-difluorophenyl]-5-(1,2-thiazol-5-yl)-1H-pyrrole-3-sulfonamide
Cpd 639 - N-[4-(difluoromethoxy)-2,5-difluorophenyl]-5-(1,2-thiazol-4-yl)-1H-pyrrole-3-sulfonamide
Cpd 640 - N-(4-cyano-2,5-difluorophenyl)-5-(furan-3-yl)-1H-pyrrole-3-sulfonamide
Cpd 641 - N-(4-cyano-2-fluorophenyl)-5-(1,3-thiazol-5-yl)-1H-pyrrole-3-sulfonamide
Cpd 642 - N-(4-cyano-2,5-difluorophenyl)-5-(1,3-thiazol-4-yl)-1H-pyrrole-3-sulfonamide
Cpd 643 - N-(4-cyano-2,5-difluorophenyl)-4-[dideuterio-(3-fluorophenyl)methyl]-1H-pyrrole-3-sulfonamide
Cpd 644 - N-(5-cyano-4-fluorothiophen-2-yl)-5-phenyl-1H-pyrrole-3-sulfonamide
Cpd 645 - N-(4-cyano-2,5-difluorophenyl)-5-(5-cyano-2-fluorophenyl)-1H-pyrrole-3-sulfonamide
Cpd 646 - N-(4-cyano-2,5-difluorophenyl)-5-(1,2-thiazol-3-yl)-1H-pyrrole-3-sulfonamide
Cpd 647 - N-(4-cyano-2,5-difluorophenyl)-5-(1,3-thiazol-5-yl)-1H-pyrrole-3-sulfonamide
Cpd 648 - 5-(5-chloro-1,3-thiazol-2-yl)-N-(4-cyano-2,5-difluorophenyl)-1H-pyrrole-3-sulfonamide
Cpd 649 - N-(4-cyano-2,5-difluorophenyl)-5-(4-methyl-1,3-thiazol-2-yl)-1H-pyrrole-3-sulfonamide
Cpd 650 - 5-(5-cyano-2-fluorophenyl)-N-[4-(difluoromethoxy)-2,5-difluorophenyl]-1H-pyrrole-3-sulfonamide
Cpd 651 - 5-(1-benzofuran-3-yl)-N-(4-cyano-2,5-difluorophenyl)-1H-pyrrole-3-sulfonamide
Cpd 652 - N-(4-cyano-2,5-difluorophenyl)-5-(3-fluoro-2-methoxyphenyl)-1H-pyrrole-3-sulfonamide
Cpd 653 - N-[2,5-difluoro-4-(trifluoromethyl)phenyl]-5-(2-methylfuran-3-yl)-1H-pyrrole-3-sulfonamide
Cpd 654 - N-(4-cyano-2,5-difluorophenyl)-5-(3-fluorophenyl)-1H-pyrrole-3-sulfonamide
Cpd 655 - 5-(5-chloro-2-fluorophenyl)-N-[3,6-difluoro-5-(2-fluoroethoxy)pyridin-2-yl]-1H-pyrrole-3-sulfonamide
Cpd 656 - N-(4-cyano-2,5-difluorophenyl)-5-(1,2-thiazol-5-yl)-1H-pyrrole-3-sulfonamide
Cpd 657 - N-(4-cyano-2,5-difluorophenyl)-5-(1,2-thiazol-4-yl)-1H-pyrrole-3-sulfonamide
Cpd 658 - N-(4-cyano-2,5-difluorophenyl)-5-(1,3-oxazol-2-yl)-1H-pyrrole-3-sulfonamide
Cpd 659 - N-[4-(difluoromethoxy)-2,5-difluorophenyl]-5-(4-methylthiophen-2-yl)-1H-pyrrole-3-sulfonamide
Cpd 660 - N-[4-(difluoromethoxy)-2,5-difluorophenyl]-5-(4-methoxythiophen-2-yl)-1H-pyrrole-3-sulfonamide
Cpd 661 - N-(4-cyano-2,5-difluorophenyl)-5-(4-methoxy-1,3-thiazol-2-yl)-1H-pyrrole-3-sulfonamide
Cpd 662 - 5-(5-cyano-2-fluorophenyl)-N-[3,6-difluoro-5-(2-fluoroethoxy)pyridin-2-yl]-1H-pyrrole-3-sulfonamide
Cpd 663 - N-(4-cyano-2,5-difluorophenyl)-5-[3-(trifluoromethyl)-1-benzofuran-7-yl]-1H-pyrrole-3-sulfonamide
Cpd 664 - 5-(5-chlorothiophen-3-yl)-N-[4-(difluoromethoxy)-2,5-difluorophenyl]-1H-pyrrole-3-sulfonamide
Cpd 665 - N-[3,6-difluoro-5-(2-fluoroethoxy)pyridin-2-yl]-5-(1,3-thiazol-2-yl)-1H-pyrrole-3-sulfonamide
Cpd 666 - 5-(4-chlorothiophen-2-yl)-N-[4-(difluoromethoxy)-2,5-difluorophenyl]-1H-pyrrole-3-sulfonamide
Cpd 667 - N-(4-cyano-2,5-difluorophenyl)-5-[5-(trifluoromethyl)-1,3-thiazol-2-yl]-1H-pyrrole-3-sulfonamide
Cpd 668 - 5-(1,3-benzothiazol-2-yl)-N-[4-(difluoromethoxy)-2,5-difluorophenyl]-1H-pyrrole-3-sulfonamide
Cpd 669 - 5-(2-chlorofuran-3-yl)-N-[2,5-difluoro-4-(trifluoromethyl)phenyl]-1H-pyrrole-3-sulfonamide

Any reference to a compound according to the present invention also includes isomers such as stereoisomers and tautomers, salts such as pharmaceutically and/or physiologically acceptable salts, hydrates, solvates, polymorphs, prodrugs, isotopes, and co-crystals of such compounds unless expressly indicated otherwise.

The term “isomers” as used herein means all possible isomeric forms, including tautomeric and stereochemical forms, which the compounds of formulae herein may possess, but not including position isomers. Typically, the structures shown herein exemplify one tautomeric or resonance form of the compounds, but the corresponding alternative configurations are contemplated as well.

Depending on its substitution pattern, the compounds of the present invention may or may not have one or more optical stereocenters and may or may not exist as different enantiomers or diastereomers. Any such enantiomers, diastereomers or other optical isomers are encompassed by the scope of the invention. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration. The terms R- or S-configuration are used herein in accordance with Chemical Abstracts nomenclature. The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

The term “pharmaceutically acceptable salts” relates to any salts that the compounds may form, and which are suitable for administration to subjects, in particular human subjects, according to the present invention. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, ethanolamine, and the like. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g., hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e., ethanedioic), malonic, succinic (i.e., butanedioic acid), maleic, fumaric, malic, tartaric, citric, methanesulfonic, ethanesulfonic, benzenesulfonic, p-toluenesulfonic, cyclohexanesulfamic, salicylic (i.e., 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of a hydrogen atom or an amino group include salts of organic carboxylic acids such as acetic, benzoic, lactic, fumaric, tartaric, maleic, malonic, malic, isethionic, lactobionic, and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX₄⁺ (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

Non-limiting examples of suitable such salts include but are not limited to acid addition salts, formed either with inorganic acids such as hydrochloric acid, hydrobromic acid, sulfuric acid, nitric acid, phosphoric acid, and the like, or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. Other salts include 2,2-dichloroacetate, adipate, alginate, ascorbate, aspartate, 2-acetamidobenzoate, caproate, caprate, camphorate, cyclamate, laurylsulfate, edisilate, esylate, isethionate, formate, galactarate, gentisate, gluceptate, glucuronate, oxoglutarate, hippurate, lactobionate, napadisilate, xinafoate, nicotinate, oleate, orotate, oxalate, palmitate, embonate, pidolate, p-aminosalicylate, sebacate, tannate, rhodanide, undecylenate, and the like; or salts formed when an acidic proton present in the parent compound is replaced, such as with ammonia, arginine, benethamine, benzathine, calcium, choline, deanol, diethanolamine, diethylamine, ethanolamine, ethylendiamine, meglumine, glycine, hydrabamine, imidazole, lysine, magnesium, hydroxyethylmorpholine, piperazine, potassium, epolamine, sodium, trolamine, tromethamine, or zinc.

The present invention includes within its scope solvates of the compounds as defined herein. The term “solvates” refers to crystals formed by an active compound and a second component (solvent) which, in isolated form, is liquid at room temperature. Such solvates may be formed with common organic solvents, e.g., hydrocarbon solvents such as benzene or toluene; chlorinated solvents such as chloroform or dichloromethane; alcoholic solvents such as methanol, ethanol, or isopropanol; ethereal solvents such as diethyl ether or tetrahydrofuran; or ester solvents such as ethyl acetate. Alternatively, the solvates of the compounds herein may be formed with water, in which case they will be hydrates.

The present invention also includes co-crystals within its scope. The term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity. Examples of co-crystal formers, which may be present in the co-crystal alongside the active pharmaceutical ingredient, include L-ascorbic acid, citric acid, glutaric acid, cinnamic acid, mandelic acid, urea, and nicotinamide.

Another embodiment of this invention relates to various precursor or “prodrug” forms of the compounds of the present invention. It may be desirable to formulate the compounds of the present invention in the form of a chemical species which itself is not significantly biologically-active, but which when delivered to the animal, mammal or human will undergo a chemical reaction catalyzed by the normal function of the body of the fish, inter al/a, enzymes present in the stomach or in blood serum, said chemical reaction having the effect of releasing a compound as defined herein. In general, such prodrugs will be functional derivatives of the compounds described herein which are readily convertible in vivo, e.g., by endogenous enzymes in the gut or the blood, into the required GPR17 modulating compounds described herein. The term “prodrug” thus relates to these species which are converted in vivo into the active pharmaceutical ingredient.

The prodrugs of the compounds of the present invention can have any form suitable to the formulator, for example, esters are non-limiting common prodrug forms. In the present case, however, the prodrug may necessarily exist in a form wherein a covalent bond is cleaved by the action of an enzyme present at the target locus. For example, a C—C covalent bond may be selectively cleaved by one or more enzymes at said target locus and, therefore, a prodrug in a form other than an easily hydrolysable precursor, inter alia an ester, an amide, and the like, may be used. The counterpart of the active pharmaceutical ingredient in the prodrug can have different structures such as an amino acid or peptide structure, alkyl chains, sugar moieties and others as known in the art.

For the purpose of the present invention the term “therapeutically suitable prodrug” can be defined herein as a compound modified in such a way as to be transformed in vivo to the therapeutically active form, whether by way of a single or by multiple biological transformations, when in contact with the tissues of the animal, mammal, or human to which the prodrug has been administered, and without undue toxicity, irritation, or allergic response, and achieving the intended therapeutic outcome.

More specifically the term “prodrug”, as used herein, relates to an inactive or significantly less active derivative of a compound such as represented by the structural formulae herein described, which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

The compound of the present invention may also exist in different crystal forms, i.e., as polymorphs and mixtures thereof, all of which are encompassed by the present invention.

The term “polymorph” refers to a particular crystalline form of a chemical compound that can crystallize in different crystalline forms, these forms having different arrangements and/or conformations of the molecules in the crystal lattice. Different crystalline forms usually have different X-ray diffraction patterns, infrared spectra, melting points, density, hardness, crystal shape, optical and electrical properties, stability, and solubility. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound described herein can exhibit beneficial effects (e.g., suitability for preparation of useful formulations, improved biological performance) relative to another polymorph or a mixture of polymorphs of the same compound. Preparation and isolation of a particular polymorph of a compound can be achieved by methods known to those skilled in the art including, for example, crystallization using selected solvents and temperatures. Recrystallization solvent, rate of crystallization, storage temperature, and other factors may cause one crystal form to dominate. Various polymorphs of a compound can be prepared by crystallization under different conditions. For a comprehensive discussion of polymorphism see Rolf Hilfiker, Ed., Polymorphism in the Pharmaceutical Industry, Wiley-VCH, Weinheim, 2006.

The invention also includes all suitable isotopic variations of a compound of the invention, which are identical to those recited in the formulas recited herein, but for the fact that one or more atoms are replaced by an atom having an atomic mass or mass number different from the atomic mass or mass number usually found in nature. An “isotopic variation”, or shortly “isotope” of a compound of the invention is defined as one in which at least one atom is replaced by an atom having the same atomic number but an atomic mass different from the atomic mass usually found in nature with the most abundant isotope(s) being preferred. Examples of isotopes that may be incorporated into compounds of the present invention include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts of said compounds or which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labelled compounds of the formulas of this invention may generally be prepared by carrying out the procedures disclosed in the examples and preparations described herein, by substituting a readily available isotopically labelled reagent for a non-isotopically labelled reagent.

Also, part of the invention are those compounds wherein at least one atom has been replaced by a radioactive isotope (radioisotope) of the same or a different atom that can be used in vivo imaging techniques such as single-photon emission computed tomography (SPECT) or positron emission tomography (PET).

Examples for such isotopic variations of GPR17 modulators usable in SPECT studies (such compounds herein “SPECT tracers”) are compounds wherein a ^{99m}Tc, ¹¹¹In, ⁸²R^{b}, ¹³⁷Cs, ¹²³I, ¹²⁵I, ¹³¹I, ⁶⁷Ga, ¹⁹²Ir or ²⁰¹Tl, and preferably ¹²³I, ^{99m}Tc or ¹¹¹In have been introduced. For example, in order for the compounds of the present invention to be used as SPECT tracers, an ¹²³I isotope may be introduced into a GPR17 modulator as disclosed herein. By way of a non-limiting example, in order for a compound to be used as SPECT tracer, a radionuclide selected from ¹²³I, ¹²⁵I and ¹³¹I may be introduced into a compound of the present invention. In one embodiment, a SPECT tracer of the present invention may be based on the structure of a halogen-containing GPR17 modulator disclosed herein, wherein one of the radionuclides ¹²³I, ¹²¹I and ¹³¹I has been introduced into the position of a halogen, preferably, an iodine atom.

Accordingly, the term “SPELT tracer of the present invention”, relates to compounds as described in the present patent application and having a structure according to anyone of Formula I, and substructures thereof further defined herein, or as otherwise individually disclosed herein, wherein at least one radioisotope has been introduced which is suitable for SPECT imaging. This includes but is not limited to ^{99m}Tc, ¹¹¹In, ⁸²R^{b}, ¹³⁷Cs, ¹²³I, ¹²⁵I, ¹³¹I, ⁶⁷Ga, ¹⁹²Ir or ²⁰¹Tl. Preferred isotopes used in the SPECT tracers of the present invention are ¹²³I, ^{99m}Tc or ¹¹¹In, preferably ¹²³I.

Examples for GPR17 modulator derivatives usable in PET applications (herein “PET tracers”) are compounds wherein ¹¹C, ¹³N, ¹⁵O, ¹⁸F, ⁷⁶Br, ¹²⁴I, ⁸²R^{b} or ⁶⁸Ga have been introduced. For example, in order for a compound to be used as a PET tracer, an ¹⁸F isotope may be introduced into a compound of the present invention. In one embodiment, a PET tracer may be based on the structure of a fluorine-containing GPR17 modulator disclosed herein, wherein the respective radionuclide ¹⁸F has been introduced into the position of the fluorine atom. This likewise applies to the introduction of at least one ¹¹C, ¹³N, ¹⁵O, ⁷⁶Br or ¹²⁴I, instead of an “unlabeled” carbon, nitrogen, oxygen, bromine, or iodine atom, respectively (see e.g., Pimlott and Sutherland, Chem Soc Rev 2011, 40, 149; van der Born et al, Chem Soc Rev 2017, 46, 4709).

Accordingly, the term “PET tracer of the present invention”, relates to compounds as described in the present patent application and having a structure according to anyone of Formula I, and substructures thereof further defined herein, or as otherwise individually disclosed herein, wherein at least one radioisotope has been introduced which is suitable for PET imaging. This includes but is not limited to ¹¹C, ¹³N, ¹⁵O, ¹⁸F, ⁷⁶Br or ¹²⁴I. Preferred PET nucleotides for use in the compounds of the present invention are ¹¹C, ¹³N, ¹⁵O, ¹⁸F, preferably ¹⁸F.

The present invention also compasses pharmaceutical compositions comprising at least one compound according to the invention, and at least one pharmaceutically acceptable carrier.

The term “pharmaceutically acceptable carrier” refers to a diluent, adjuvant, excipient, or carrier, or other ingredient with which a compound of the invention is administered and which a person of skilled in the art would understand to be pharmaceutically acceptable.

Tablets will contain excipients, glidants, fillers, binders, and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid, and the like.

Subsequently, the term “pharmaceutically acceptable carrier” as used herein means any material or substance with which the active ingredient is formulated in order to facilitate its application or dissemination to the locus to be treated, for instance by dissolving, dispersing, or diffusing the said composition, and/or to facilitate its storage, transport, or handling without impairing its effectiveness. The pharmaceutically acceptable carrier may be a solid or a liquid or a gas which has been compressed to form a liquid, e.g., the compositions of this invention can suitably be used as concentrates, emulsions, solutions, granulates, dusts, sprays, aerosols, suspensions, ointments, creams, tablets, pellets, or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, e.g., carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronization, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g., the sodium or potassium salts of oleic or stearic acid, or of natural fatty acid mixtures obtainable from coconut oil or tallow oil. Synthetic surfactants include sodium or calcium salts of polyacrylic acids; fatty sulfonates and sulfates; sulfonated benzimidazole derivatives and alkylarylsulfonates. Fatty sulfonates or sulfates are usually in the form of alkaline or alkaline-earth metal salts, unsubstituted ammonium salts or ammonium salts substituted with an alkyl or acyl group having from 8 to 22 carbon atoms, e.g., the sodium or calcium salt of lignosulfonic acid or dodecylsulfonic acid or a mixture of fatty alcohol sulfates obtained from natural fatty acids, alkaline or alkaline-earth metal salts of sulfuric or sulfonic acid esters (such as sodium lauryl sulfate) and sulfonic acids of fatty alcohol/ethylene oxide adducts. Suitable sulfonated benzimidazole derivatives preferably contain 8 to 22 carbon atoms. Examples of alkylarylsulfonates are the sodium, calcium or alcoholamine salts of dodecylbenzene sulfonic acid or dibutyl-naphthalenesulfonic acid or a naphthalene-sulfonic acid/formaldehyde condensation product. Also suitable are the corresponding phosphates, e.g., salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g., phosphatidylethanolamine, phosphatidylserine, phosphatidylglycerine, lysolecithin, cardiolipin, dioctanylphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulfonates and dialkylsulfosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol, and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon groups optionally substituted with halogen, phenyl, substituted phenyl or hydroxy; for instance, quaternary ammonium salts containing as N-substituent at least one C₈₋₂₂alkyl (e.g., cetyl, lauryl, palmityl, myristyl, oleyl, and the like) and, as further substituents, unsubstituted or halogenated lower alkyl, benzyl and/or hydroxy-lower alkyl.

A more detailed description of surface-active agents suitable for this purpose may be found for instance in “McCutcheon's Detergents and Emulsifiers Annual” (MC Publishing Crop., Ridgewood, New Jersey, 1981), “Tensid-Taschenbucw”, 2 d ed. (Hanser Verlag, Vienna, 1981) and “Encyclopaedia of Surfactants, (Chemical Publishing Co., New York, 1981).

Compounds of the invention and their pharmaceutically acceptable salts (hereafter collectively referred to as the active ingredients) may be administered by any route appropriate to the condition to be treated, suitable routes including oral, rectal, nasal, topical (including ocular, buccal, and sublingual), vaginal and parenteral (including subcutaneous, intramuscular, intravenous, intradermal, intrathecal, and epidural). The preferred route of administration may vary with for example the condition of the recipient.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal, and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets, or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, a polyhydric alcohol, e.g., an alcohol having two or more hydroxyl groups such as propylene glycol, butane 1,3-diol, mannitol, sorbitol, glycerol, and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus, the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 μm (including particle sizes in a range between 20 and 500 μm in increments of 5 μm such as 30 μm, 35 μm, etc.), which is administered in the manner in which snuff is taken, e.g., by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foam, or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g., microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on.

Depending on the route of administration, the pharmaceutical composition may require protective coatings. Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g., one of them may be in the form of an oral or parenteral formulation whereas the other is in the form of an ampoule for intravenous injection or an aerosol.

The compounds of the present invention are useful in the prevention and/or treatment of certain GPR17 mediated diseases or disorders in subjects such as animals, in particular in humans, as described herein.

The term “preventing” or “prevention” as used herein refers to a reduction in risk of acquiring a disease or disorder (i.e., causing at least one of the clinical symptoms of the disease not to develop in a subject, in particular a human subject, that may be exposed to or predisposed to the disease but does not yet experience or display symptoms of the disease).

The term “treating” or “treatment” of any disease or disorder includes, in one embodiment, to improve the disease or disorder (i.e., arresting or reducing the development of the disease or at least reducing one of the clinical symptoms of the disease). In another embodiment “treating” or “treatment” refers to improve at least one physical parameter, which may or may not be discernible by the subject, in particular a human subject, but which is based on or associated with the disease or disorder to be treated. In yet another embodiment, “treating” or “treatment” refers to modulating or alleviating the disease or disorder, either physically (e. g. stabilization of a discernible on non-discernible symptom), physiologically (e. g. stabilization of a physiological parameter), or both. In yet another embodiment, “treating” or “treatment” refers to delaying the onset or progression of the disease or disorder. Accordingly, “treating” or “treatment” includes any causal treatment of the underlying disease or disorder (i.e., disease modification), as well as any treatment of signs and symptoms of the disease or disorder (whether with or without disease modification), as well as any alleviation or amelioration of the disease or disorder, or its signs and symptoms. The terms “disease(s)” and “disorders)” are used largely interchangeably herein.

The term “diagnosis”, “diagnoses” or “diagnosing” of a disease or disorder, as used herein, include, in one embodiment, the identification and measurement of signs and symptoms which are associated with said disease. “Diagnosis”, “diagnoses” or “diagnosing” include but are not limited to the detection and/or measurement of decreased, increased, or otherwise incorrectly (e.g., as to time or place) expressed, activated, or distributed GPR17 receptors as indicator of a GPR17-related disease or disorder, as compared to healthy subjects. In one example, GPR17 ligands may be used in the form of PET or SPECT tracers for such a diagnosis, including a diagnosis for a myelination disease.

The term “subject” refers to an animal preferably a mammalian patient in need of such treatment, such as a human. The term also refers to an animal, preferably a mammal, most preferably a human, who has been the object of treatment, observation, or experiment. The terms “human”, “patient” and “human subject” are typically used interchangeably herein, unless clearly indicated. The invention also relates to methods of treating an animal disease or disorder, as described in more detail herein, in particular a human disease or disorder, which includes the administration of the compounds of the present invention in therapeutically effective amounts.

The term “therapeutically effective amount” as used herein, means that amount of active compound or pharmaceutical agent that, when administered to a subject, elicits the biological or medicinal response in a tissue system, or a subject that is being sought by a researcher, veterinarian, medical doctor, or other clinician, which includes alleviation or partial alleviation of the symptoms of the disease or disorder being treated. The therapeutically effective amount can vary depending on the compound, the disease and its severity, and the condition, age, weight, gender etc. of the subject, in particular a human subject, to be treated.

The compounds of the invention are GPR17 modulators. The term “GPR17 modulators” as used herein are meant to describe compounds that are capable of modulating the activity of the GPR17 receptor, in particular compounds that are capable of decreasing the GPR17 activity. Such “negative GPR17 modulators” include GPR17 antagonists which are capable of blocking the effects of GPR17 ligands, as well as GPR17 inverse agonists which are capable of inhibiting constitutive active GPR17 receptors or receptor variants.

Because of their GPR17 modulating properties, the compounds of the present invention can be used as medicine. The present invention therefore encompasses the compounds of the invention for use as a medicine, and preferably for use in the prevention and/or treatment or diagnosis of a GPR17 mediated disorder.

A GPR17 mediated disease or disorder can be defined as disease which is associated with a dysfunction of the GPR17 signaling system such as, for example, an overexpression and/or overactivity of GPR17 receptors.

The present compounds may be used for example for the treatment and/or prevention of various diseases of the CNS system. CNS disorders include disorders of the CNS as well as disorders of the peripheral nervous system.

Without wished to be bound by any theory, the activity of GPR17 may be increased, extended, or otherwise altered in certain tissues, for example in oligodendrocyte progenitor cells (OPCs) or during maturation of oligodendrocytes, potentially due to activating endogenous stimuli such as, for example, inflammation factors. High activity of GPR17 may prevent the differentiation of oligodendrocytes and an efficient myelination, thus promoting the emergence or further development of a myelination disease. Negative GPR17 modulators may thus promote myelination by decreasing or turning off GPR17 activity and by supporting OPC maturation into myelin-producing oligodendrocytes (Simon et al., J Biol Chem. 2016 Jan. 8; 291(2):705-18).

The present invention therefore encompasses compounds described herein, for use in the prevention or treatment of a disorder or syndrome selected from and/or associated with a myelination disorder, in particular a demyelination disorder, such as of the CNS. In one embodiment, the compounds of the present invention are for use in promoting, stimulating and/or accelerating remyelination or myelination in an animal in need thereof. In one embodiment, the remyelination associated with the administration of a compound of the present invention will prevent or treat a demyelination disease such as, but not limited to, multiple sclerosis.

Compounds of the present invention can also be useful in the treatment or prevention of a disorder or syndrome associated with brain tissue damage, a cerebrovascular disorder, and certain neurodegenerative diseases. Neurodegenerative disorders have been recently associated strongly with a loss of myelination. Accordingly, it is believed that preserved oligodendroglial and myelin functionality is a crucial prerequisite for the prevention of axonal and neuronal degeneration (Ettle et al., Mol Neurobiol. 2016; 53(5): 3046-3062). The present compounds may thus represent an excellent treatment option for any neurodegenerative disease associated with demyelination and/or impacted myelination such as e.g., ALS, MSA, Alzheimer's disease, Huntington Disease or Parkinson's Disease.

In a particular preferred embodiment, the compounds of the present invention can thus be used in the prevention and/or treatment of a peripheral or central myelination disorder, in particular of a myelination disorder of the CNS. In one aspect, the compounds of the present invention are used in the treatment and/or prevention and/or diagnosis of a myelination disorder by oral administration. In a preferred embodiment, the myelination disorder to be treated with the compounds of the present invention is a demyelination disorder.

Non-limiting examples of such myelination disorders to be treated and/or prevented by the presently disclosed compounds are, in particular,
Multiple sclerosis (MS) including its various subforms
Optic neuritis
Neuromyelitis optica (also known as Devic's disease)
Chronic relapsing inflammatory optic neuritis, acute disseminated bencephalomyelitis
Acute hemorrhagic leucoencephalitis (AHL)
Periventricular leukomalacia demyelination due to viral infections, e.g., by HIV or progressive multifocal leukoencephalopathy
Central pontine and extrapontine myelinolysis
Demyelination due to traumatic brain tissue damage, including compression induced demyelination, e.g., by tumors demyelination in response to hypoxia, stroke or ischemia or other cardiovascular diseases
Demyelination due to exposure to carbon dioxide, cyanide, or other CNS toxins
Schilder's disease
Balo concentric sclerosis
Perinatal encephalopathy
Neurodegenerative Diseases including, in particular:
   Amyotrophic lateral sclerosis (ALS)
   Alzheimer's disease (AD)
   Multiple system atrophy
   Parkinson's Disease
   Spinocerebellar ataxia (SCA), also known as spinocerebellar atrophy
   Huntington's Disease
Psychiatric disorders such as schizophrenia and bipolar disorder (Fields, Trends Neurosci. 2008 July; 31(7): 361-370; Tkachev et al., Lancet. 2003 Sep. 6; 362(9386):798-805).
Peripheral myelination diseases such as leukodystrophies, peripheral demyelinating neuropathies, Dejerine-Sottas syndrome or Charcot-Marie-Tooth disease

The treatment or prevention of a CNS disease such as a demyelination disease, also includes the treatment of the signs and symptoms associated with such a disease. For example, the use of the compounds of the present invention for the treatment and/or prevention of MS also includes the treatment and/or prevention of the signs and symptoms associated with MS such as negative effects on optic nerves (vision loss, double vision), dorsal columns (loss of sensation), corticospinal tract (spastic weakness), cerebellar pathways (incoordination, dysarthria, vertigo, cognitive impairment), medial longitudinal fasciculus (double vision on lateral gaze), spinal trigeminal tract (face numbness or pain), muscle weakness (impaired swallowing, control of the bladder or gut, spasms), or psychological effects associated with the underlying disease such as depression, anxiety or other mood disorders, general weakness or sleeplessness. Hence, the compounds of the present invention are suitable for use in treating signs and symptoms of a myelination disease, in particular a demyelination disease such as multiple sclerosis; such signs and symptoms of MS include but are not limited to the group of vision loss, vision impairment, double vision, loss or impairment of sensation, weakness such as spastic weakness, motor incoordination, vertigo, cognitive impairment, face numbness, face pain, impaired swallowing, impaired speech, impaired control of bladder and/or gut, spasms, depression, anxiety, mood disorders, sleeplessness, and fatigue. In one preferred embodiment, the compounds of the present invention are for use in treating multiple sclerosis. MS is a heterogeneous myelination disease and can manifest itself in a variety of different forms and stages, including but not limited to Relapsing Remitting MS, Secondary-Progressive MS, Primary Progressive MS, Progressive Relapsing MS, each depending on activity and disease progression. Hence, in some embodiments, the compounds of the present invention are suitable for use in treating multiple sclerosis in its various stages and forms, as described herein. In some embodiments, the compounds of the present invention are for use in the treatment/or prevention of Neuromyelitis optica (also known as Devic's disease or Devic's syndrome). Neuromyelitis optica is a complex disorder characterized by inflammation and demyelination of the optic nerve and the spinal cord. Many of the associated symptoms are similar to MS and include muscle weakness, in particular of the limbs, reduced sensation and loss of bladder control.

In some embodiments, the compounds of the present invention are suitable for use in prevention and/or treating ALS. ALS has been associated recently with oligodendrocyte degeneration and increased demyelination, suggesting ALS as a target disease for negative GPR17 modulators (Kang et al., Nature Neurosci 16, 2013, 571-579; Fumagalli et al., Neuropharmacology. 2016 May; 104:82-93). In some embodiments, the compounds of the present invention are for use in prevention and/or treating Huntington Disease. Huntington is well described to be associated with impacted myelination, (Bartzokis et al., Neurochem Res. 2007 October; 32(10):1655-64; Huang et al., Neuron. 2015 Mar. 18; 85(6): 1212-1226).

In some embodiments, the compounds of the present invention are for use in prevention and/or treating multiple system atrophy (MSA), which was recently associated strongly with demyelination (Ettle et al., Mol Neurobiol. 2016; 53(5): 3046-3062; Jellinger and Welling, Movement Disorders, 31, 2016; 1767), suggesting remyelination strategies to treat or prevent MSA.

In some embodiments, the compounds of the present invention are for use in prevention and/or treating Alzheimer's Disease. AD has been recently observed to be associated with increased cell death of oligodendrocytes and focal demyelination and to represent a pathological process in AD (Mitew et al., Acta Neuropathol. 2010 May; 119(5):567-77).

The present invention also encompasses a compound as described herein for use in a method of treatment of anyone of the diseases or disorders described herein, in particular of a myelination disease such as MS, optic neuritis, Neuromyelitis optica, ALS, Chorea Huntington, AD or others, by administering to a subject in need thereof, including a human patient, a therapeutically effective amount of a compound of the present invention.

In some embodiments, the compound of the present invention may be used in the prevention and treatment of a spinal cord injury, perinatal encephalopathy, stroke, ischemia, or a cerebrovascular disorder.

The present invention also encompasses a compound as described herein for use in a method for the prevention and/or treatment of a syndrome or disorder associated with a myelination disorder, or with a disorder or syndrome associated with a brain tissue damage, which comprises administering to a patient in need thereof a therapeutically effective amount of a compound as described herein. A patient in need of such a treatment can be any patient who suffered brain tissue damage such as by mechanical, chemical, viral, or other trauma.

In some embodiments, the compound as described herein is suitable for use in a method for the prevention and/or treatment of a syndrome or disorder associated with a myelination disorder, or with a disorder or syndrome associated with stroke or other brain ischemia, which comprises administering to a patient in need thereof a therapeutically effective amount of a compound as described herein. A patient in need thereof may be any patient that recently experienced a cerebral ischemia/stroke which may have been caused, for example, by the occlusion of a cerebral artery either by an embolus or by local thrombosis.

GPR17 has been also associated with food uptake, insulin control and obesity recently. According to various reports, negative modulators of GPR17 may be helpful for controlling food uptake and for treating obesity (see e.g., Ren et al., Diabetes 2015 November; 64(11): 3670-3679). Hence, the present invention also encompasses the compounds described herein for use in the prevention and/or treatment of obesity, and methods of treating obesity.

Moreover, the compounds of the present invention may be used for the treatment of prevention of tissues where GPR17 is expressed, such as e.g., heart, lung, or kidney. In some embodiments, the compounds of the present invention can be used to treat or prevent ischemic disorders of the kidney and/or the heart.

GPR17 has been also associated with pulmonary inflammation and asthma such as, for example, induced by house dust mite (Maekawa et al., J Immunol Aug. 1, 2010, 185 (3) 1846-1854). Hence, the compounds of the present invention may be used for the treatment of asthma or other pulmonary inflammation.

The treatment according to the invention may comprise the administration of one of the presently disclosed compounds as “stand alone” treatment of a GPR17 mediated disorder, such as a CNS disease, in particular of a myelination disease or disorder such as MS or ALS. Alternatively, a compound disclosed herein may be administered together with other useful drugs in a combination therapy.

In a non-limiting example, a compound according to the present invention can be combined with another medicament for treating a GPR17 mediated disorder, such as a myelination disease, such as MS, said other medication having for example a different but complementary mode of action, such as e.g., an anti-inflammatory or immunosuppressive drug. Non-limiting examples of such compounds include (i) corticosteroids such as prednisone, methylprednisolone or dexamethasone, (ii) beta interferons such as interferon beta-1a, interferon beta-1b or peginterferon beta-1a, (iii) anti-CD20 antibodies such as ocrelizumab rituximab and ofatumumab, (iv) glatiramer salts such as glatiramer acetate, (v) dimethyl fumarate, (vi) fingolimod and other sphingosine-1-phosphate receptor modulators such as ponesimod, siponimod, ozanimod or laquinimod, (vii) dihydro-orotate dehydrogenase inhibitors such as teriflunomide or leflunomide, (viii) anti-integrin alpha4 antibodies such as natalizumab, (ix) anti CD52 antibodies such as alemtuzumab, (x) mitoxantrone, (xi) anti-Ling antibodies such as opicinumab, or (xii) other immunomodulatory therapies such as masitinib. Likewise, a compound of the present invention can be combined with an analgesic drug if a painful myelination condition is to be treated. Also, a compound of the present disclosure may be used in combination with an anti-depressant to co-treat psychological effects associated with the underlying myelination disease to be treated.

In combination therapies the two or more active principles may be provided via the same Formulation or as a “kit of parts”, i.e., in separate galenic units. Also, the two or more active principles, including the compounds of the present invention, may be administered to the patient at the same time or subsequently, e.g., in an interval therapy. The additional drug may be administered by the same mode or a different mode of administration.

In, some embodiments, the compounds of the present invention may be used for the diagnosis and/or monitoring of a GPR17-related disease, as further described herein, in particular of a demyelinating disease, as disclosed herein, preferably in the diagnosis and monitoring of multiple sclerosis.

In some embodiments, the compounds of the present invention can be used to diagnose and/or monitor the expression, distribution and/or activation of the GPR17 receptor either in vivo, e.g., directly in a subject, such as using molecular imaging techniques, or in vitro, such as e.g., by examining any samples such as body fluids or tissues taken from a subject. Any such determination of the GPR17 activity, expression and/or distribution may be used to predict, diagnose and/or monitor (a) the status and progression of a GPR17-associated disease as described herein, in particular a myelination disease including but not limited to, for example, multiple sclerosis, and (b) the efficacy and/or applicability and/or proper dosing of a treatment associated with any such GPR17-associated disease.

In some embodiments, the compounds of the present invention may be used as PET or SPECT tracers, as further disclosed herein, in order to perform in vivo diagnosis and/or disease monitoring. By this, the expression, activation and/or distribution of a GPR17 receptor may be directly measured in a subject, e.g., by imaging of a human patient after the administration of a GPR17 PET or SPECT tracer of the present invention. This may facilitate a proper diagnosis of the disease, can help to determine applicable treatment options and/or may be used to monitor disease progression and/or to monitor or predict the success of a medical intervention, including the selection and proper administration and/or dosing of a therapeutic drug.

In some embodiments, the PET or SPECT tracers of the present invention may be used in conjunction with a therapeutic drug, i.e., as a companion diagnostic, in order to monitor and/or predict the efficacy and/or safety of said therapeutic drug in a particular subject, or to estimate a drug's proper dosage.

The therapeutic drug to be used with the PET or SPECT tracer of the present invention may be selected from the group of (a) an unlabeled compound of the present invention, (b) a GPR17 modulating compound which is different from the compounds of the present invention and (c) a drug for the treatment of a myelination disease, including but not limited to a drug for use in multiple sclerosis treatment, which is not a GPR17 modulator, as further described herein.

One embodiment relates to a kit comprising (a) as a first component, a PET or SPECT tracer of the present invention, (b) as a second component, a therapeutic drug selected from among i. a compound of the present invention and having no radionuclide incorporated, ii. a GPR17 modulating compound which is different from the compounds of the present invention as defined in (i), and iii. a drug for the treatment of a myelination disease, including but not limited to a drug for use in multiple sclerosis treatment, but having no GPR17 modulating activity; such compounds are known to a person skilled in the art including those examples further described above.

Alternatively, the compounds of the present invention may be used in an in vitro diagnostic assay, for example for the examination of suitable body fluids of a subject such as e.g., blood, plasma, urine, saliva, or cerebrospinal fluid for any level of GPR17 expression, activity and/or distribution.

The compounds of the invention can be prepared while using a series of chemical reactions well known to those skilled in the art, altogether making up the process for preparing said compounds and exemplified further. The processes described further are only meant as examples and by no means are meant to limit the scope of the present invention.

Abbreviations used in the description, particularly in the Schemes and Examples, are as follows: AcOH—Acetic acid, AcOK—Potassium acetate, ADDP—1,1′-(Azodicarbonyl)dipiperidine, aq.—Aqueous, Boc—ter-Butoxycarbonyl, [bmim][BF₄]-1-Butyl-3-methylimidazolium tetrafluoroborate, Boc₂O—Di-tert-butyl dicarbonate, COMU—(1-Cyano-2-ethoxy-2-oxoethylidenaminooxy)-dimethylamino-morpholino-carbenium hexafluorophosphate, DAST—Diethylaminosulfur trifluoride, DBU—1,8-Diazabicyclo-[5.4.0]undec-7-ene, DCC—N,N′-dicyclohexylcarbodiimide, DCE—1,2-dichloroethane, DCM—Dichloromethane, DEAD—Diethyl azodicarboxylate, DEA—Diethylamine, DIPEA—Diisopropyl-ethyl amine, DIA—Diastereomer, DIAD—Diisopropyl azodicarboxylate, DMAc—Dimethylacetamide, DMAP—N,N-Dimethylpyridin-4-amine, DME—1,2-Dimethoxyethane, DMF—N,N-Dimethylformamide, DMSO—Dimethylsulfoxide, DTBAD—tert-Butylazodicarboxylate, EDC.HCl—1-Ethyl-3-(3-di methylaminopropyl)carbodiimide hydrochloride, En—Enantiomer, Et₂O—Diethyl ether, EtOAc—Ethyl acetate, EtOH—Ethanol, Eq.—Equivalent, FA—Formic acid, FCC—Flash column chromatography, GCMS—Gas chromatography-mass spectrometry, h—Hour, HATU—O-(7-Azabenzotriazol-1-yl)-N,N,N′,N′-tetramethyluronium hexafluorophosphate, HOBT—1-hydroxybenzotriazole hydrate, HMPA—Hexamethylphosphoramide, HPLC—High performance liquid chromatography, IPA—isopropyl alcohol, i-PrMgCl—Isopropylmagnesium chloride, [IrCp*Cl₂]₂—Pentamethylcyclopentadienyliridium(III) chloride, dimer, [Ir{dF(CF₃)ppy}₂(dtbpy)]PF₆—[4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate, LCMS—Liquid chromatography-mass spectrometry, LG—Leaving group, MeCN (CH₃CN)—ACN—Acetonitrile, MeOH—Methanol, MgSO₄—Magnesium sulfate, min.—Minute, MeONa—Sodium methoxide, MOMCl—Chloromethyl methyl ether, Na₂SO₄—Sodium sulfate, NBS—N-Bromosuccinimide, NCS—N-Chlorosuccinimide, NFSI—N-Fluorobenzenesulfonimide, NIS—N-lodosuccinimide, NMP—1-Methyl-2-pyrrolidinone, NMR—Nuclear Magnetic Resonance, Pd(PPh₃)₄-Tetrakis-(triphenylphosphine)-palladium(0), Pd/C—Palladium on carbon, PdCl₂(PPh₃)₂-Bis(triphenylphosphine)palladium(II) dichloride, Pd₂(dba)₃—Tris(dibenzylideneacetone)dipalladium, Pd(amphos)Cl₂—Bis(di-tert-butyl(4-dimethylaminophenyl)phosphine)dichloropalladium(II), Pd(OAc)₂—Palladium(II) acetate, Pd(dppf)Cl₂—[1,1′-Bis(diphenylphosphino)ferrocene]dichloropalladium(II), Pd(dppf)Cl₂·CH₂Cl₂—CH₂Cl₂, [1,1′-Bis(diphenylphosphino)ferrocene]dichloropalladium(II) (1:1), PE—Petrol ether, PMB-Cl—4-Methoxybenzyl chloride, PPh₃—Triphenylphospine, PS-DIEA—Diisoprpropyl-ethyl amine supported on PolyStyrene, PS-PPh₃—Triphenylphospine supported on PolyStyrene, PyBop—Benzotriazol-1-yl-oxytripyrrolidinophosphonium hexafluorophosphate, Py·SO₃—Sulfur trioxide pyridine complex, R^{p}—Reverse phase, RI—Room temperature, RM—Reaction mixture, sat.—Saturated, SFC—Supercritical fluid chromatography, SPE—Solid Phase Extraction, t-BuLi—tert-Butyl lithium, t-BuOK—Potassium t-butoxide, TBAF—Tetrabutylammonium fluoride, TBAI—Tetrabutylammonium iodide, tBuONO—tert-Butyl nitrite, TFA—Trifluoroacetic acid, TFAA—Trifluoroacetic anhydride, THF—Tetrahydrofuran, TIPS—Triisopropylsilyl, TLC—Thin Layer Chromatography, TMSNTf₂—N-(Trimethylsilyl)bis(trifluoromethanesulfonyl)imide, Ts—Tosyl, TsOH—para-Toluenesulfonic acid, TsCl—p-toluenesulfonyl chloride, XPhos—2-Dicyclohexylphosphino-2′,4′,6′-tri isopropylbiphenyl.

In some embodiments, the compounds of the present invention may be prepared according to the general procedures outlined in Scheme 1.

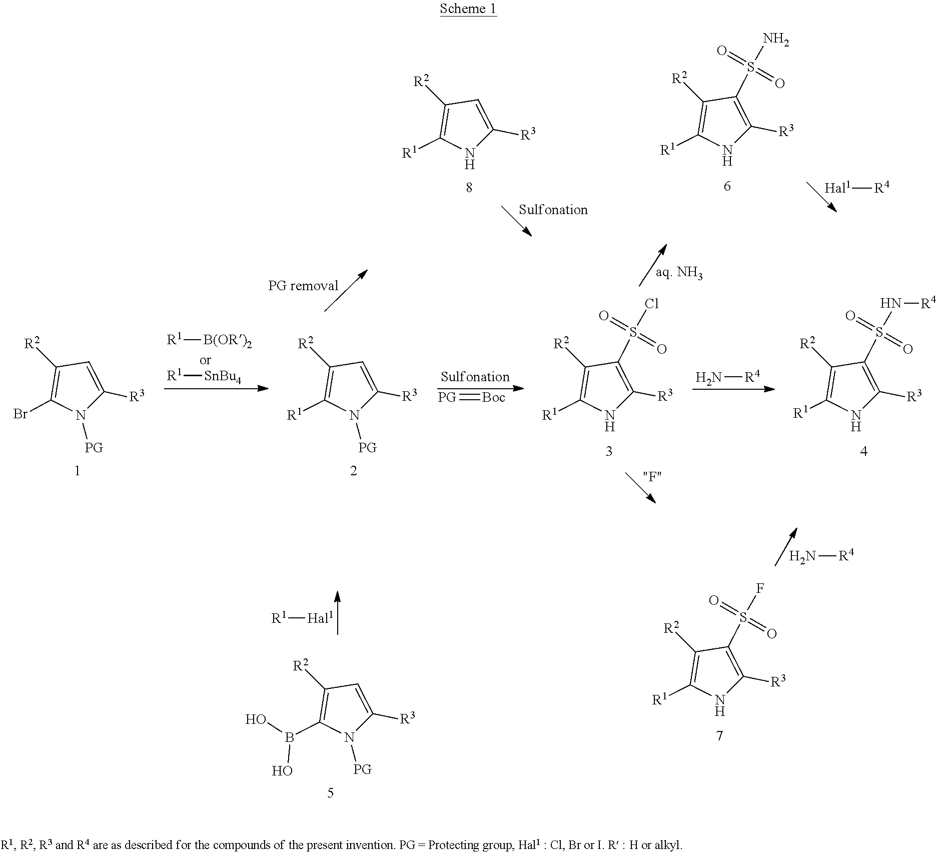

2-Bromo-pyrrole of formula 1, wherein PG is a protecting group (e.g., Boc or Ts), commercially available or synthesized by procedures known to the skilled in the art or as set forth examples below, may be coupled with a boronic acid, boronic ester or a tin derivative (commercially available or synthesized by procedures known to the person skilled in the art) in presence of a palladium catalyst (e.g., Pd(PPh₃)₄, Pd(dppf)Cl₂ and the like) and a salt (e.g., KF, K₃PO₄, Na₂CO₃ and the like) in a solvent or mixture of solvents (e.g., DMF, toluene, dioxane, water, and the like) at a temperature ranging from 0 to 100° C. to provide intermediates of formula 2. Alternatively, the compound of general formula 2 may be obtained via a Suzuki coupling between the boronic acid (commercially available or synthesized by procedures known to the person skilled in the art) and R¹-Hal¹ (commercially available or synthesized by procedures known to the person skilled in the art). Pyrrole of formula 3 may be directly obtained from compound of general formula 2 (with PG: Boc) using a sulfonyl-chlorinating agent (e.g., Chlorosulfonic acid and the like) in a polar solvent (e.g., MeCN and the like) at a temperature ranging from 0 to 120° C. Alternatively, the compound of general formula 3 may be obtained from compound of general formula 2 (with PG: Boc) using a sulfonating agent (e.g., SO₃, Py·SO₃ and the like) in a polar solvent (e.g., MeCN, DCM and the like) at a temperature ranging from 0 to 120° C. followed by a subsequent reaction with a chlorination reagent (e.g., POCl₃, thionyl chloride, oxalyl chloride and the like) in a polar solvent (e.g., MeCN, DCM and the like) at a temperature ranging from 0 to 120° C. Alternatively, compound of general formula 2, wherein PG is a protecting group (e.g., Boc or Ts), may be deprotected following procedures known to the skilled in the art (e.g., treatment with a base such as Na₂CO₃ if PG=Ts or in presence of an acid (e.g., HCl, TFA and the like) if PG=Boc) to provide the compound of general formula 8. Pyrrole of formula 3 may be directly obtained from compound of general formula 8 using a sulfonyl-chlorinating agent (e.g., chlorosulfonic acid and the like) in a polar solvent (e.g., MeCN and the like) at a temperature ranging from 0 to 120° C. Alternatively, the compound of general formula 3 may be obtained from compound of general formula 8 using a sulfonating agent (e.g., SO₃, Py·SO₃ and the like) in a polar solvent (e.g., MeCN, DCM and the like) at a temperature ranging from 0 to 120° C. followed by a subsequent reaction with a chlorination reagent (e.g., POCl₃, thionyl chloride, oxalyl chloride and the like) in a polar solvent (e.g., MeCN, DCM and the like) at a temperature ranging from 0 to 120° C. Sulfonyl chloride derivative 3 may be condensed with an amine (R⁴—NH₂) with or without a base (e.g., NaH, Pyridine and the like) in a solvent (e.g., THF, Pyridine, MeCN and the like) to afford compounds of interest of generic formula 4. Alternatively, Pyrrole-3-sulfonamide 4 may be prepared by condensation of Sulfonyl chloride derivative 3 with ammonia solution (aq. NH₃) in a solvent (e.g., THF and the like) followed by a subsequent coupling type reaction of intermediates of general formula 6 with an halogenated compound of formula Hal¹-R⁴ in the presence of a catalyst (e.g., CuI and the like), a ligand (e.g., trans-N,N-dimethylcyclohexane-1,2-diamine and the like), a base (e.g., K₂CO₃ and the like) and a polar solvent (e.g., MeCN and the like). Alternatively, Pyrrole-3-sulfonamide 4 may be prepared via fluorination of Sulfonyl chloride derivative 3 with a fluorinated agent (e.g., KF, TBAF and the like) in a solvent (e.g., THF and the like) followed by a subsequent condensation with an amine (R⁴—NH₂) in a presence of a Lewis Acid (e.g., TMSNTf₂, TMSOTf and the like) in a solvent (e.g., Pyridine, and the like) at a temperature ranging from 0 to 120° C.

In another embodiment, compounds of the present invention may also be synthesized according to the general procedure outlined in Scheme 2.

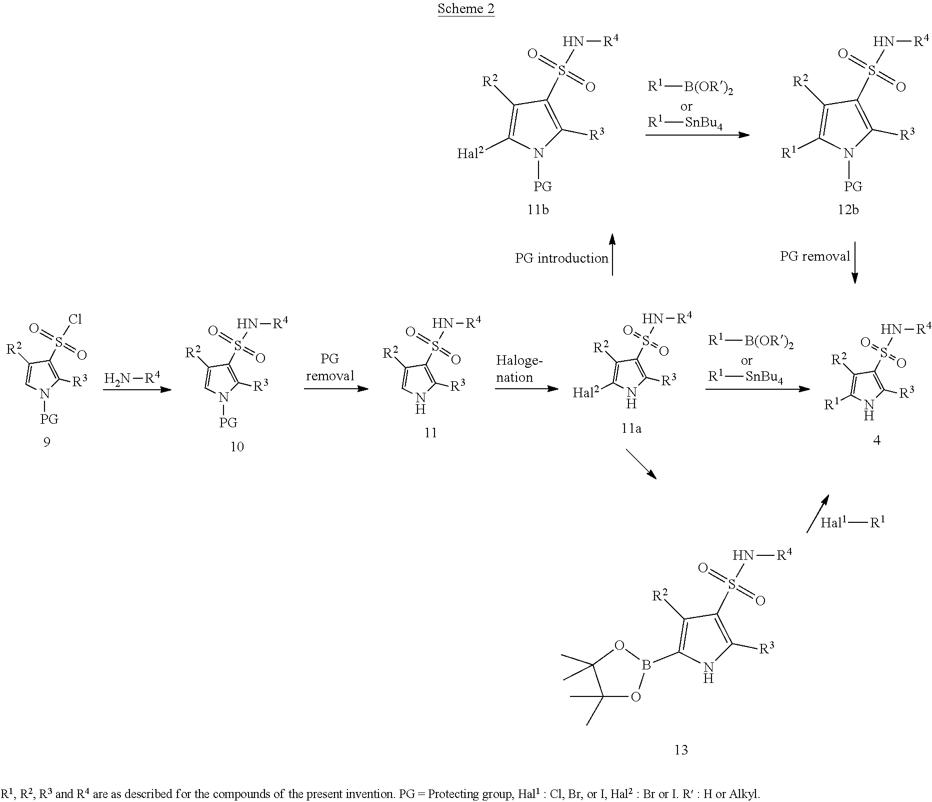

Pyrrole-3-sulfonyl chloride compounds of formula 9, wherein PG is a protecting group (e.g., Boc or Ts), commercially available or synthesized by procedures known to the skilled in the art or as set forth examples below, may be condensed with an amine (R⁴—NH₂) with or without a base (e.g., NaH, Pyridine and the like) in a solvent (e.g., THF, Pyridine, MeCN and the like) to provide intermediates of formula 10. Pyrrole intermediates of formula 11 may be obtained by deprotection of an intermediate 10 following procedures known to the skilled in the art (e.g., treatment with a base (e.g., Na₂CO₃ or LiOH and the like) if PG=Ts or in presence of an acid (e.g., HCl, TFA and the like) if PG=Boc). Halogenated pyrroles of formula 11a wherein Hale can be iodine or bromine, may be obtained by bromination or iodination of compounds 11 in presence of a halogenating agent (e.g., NBS, NIS and the like) in a polar solvent (e.g., DMF and the like) following procedures known to the skilled in the art. Halogenated pyrroles of formula 11a may be coupled with a boronic acid, boronic ester or a tin derivative (commercially available or synthesized by procedures known to the person skilled in the art) in presence of a palladium catalyst (e.g., Pd(PPh₃)₄, Pd(dppf)Cl₂ and the like) and a salt (e.g., KF, K₃PO₄, Na₂CO₃ and the like) in a solvent (e.g., DMF, toluene, dioxane, water, and the like) at a temperature ranging from 0 to 100° C. to provide the desired compounds of formula 4. Alternatively, 11a may be converted in a boronic esters of general formula 13, via a Miyaura Borylation Reaction (For an article of such methods, see e.g., T. Ishiyama, M. Murata, N. Miyaura, J. Org. Chem., 1995, 60, 7508-7510). The desired compounds of general formula 4 may be obtained via a Suzuki coupling between a boronic ester 13 and a halogenated reagent Hal¹-R¹ (commercially available or synthesized by procedures known to the person skilled in the art).

Alternatively, 11a may be converted in a protected pyrrole of formula 11 b, following procedures known to the person skilled in the art (e.g., treatment with TsCl, Boc₂O, (i-Pr)₃SiCl, in the presence of a base (e.g., NaH, Et₃N, DMAP and the like) and in solvent (e.g., THF, DCM, MeCN and the like)). Halogenated pyrroles of formula lib may be then coupled with a boronic acid, boronic ester or a tin derivative (commercially available or synthesized by procedures known to the person skilled in the art) in presence of a palladium catalyst (e.g., Pd(PPh₃)₄, Pd(dppf)Cl₂ and the like) and a salt (e.g., KF, K₃PO₄, Na₂CO₃ and the like) in a solvent (e.g., DMF, toluene, dioxane, water, and the like) at a temperature ranging from 0 to 100° C. to provide the desired compounds of formula 12b. Pyrroles of formula 4 may be obtained by deprotection of a compound 12b following procedures known to the skilled in the art (e.g., treatment with a base (e.g., Na₂CO₃ or LiOH and the like) if PG=Ts or in presence of an acid (e.g., HCl, TFA and the like) if PG=Boc).

In another embodiment, compounds of the present invention may also be synthesized according to the general procedure outlined in Scheme 2a.

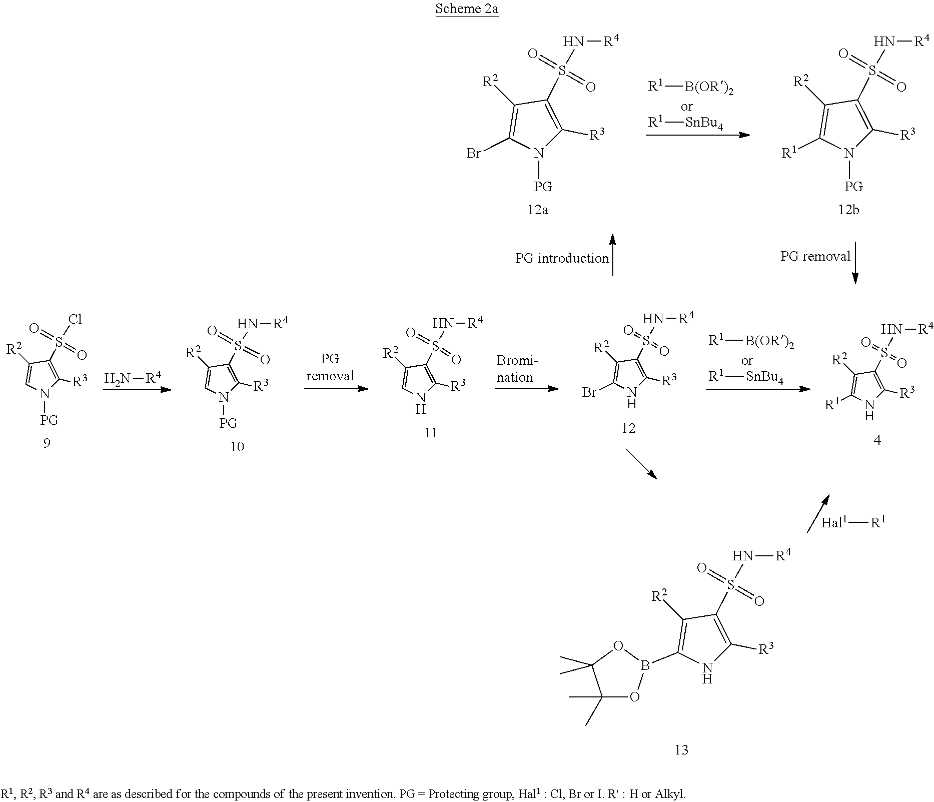

Pyrrole-3-sulfonyl chloride of formula 9, wherein PG is a protecting group (e.g., Boc or Ts), commercially available or synthesized by procedures known to the skilled in the art or as set forth examples below, may be condensed with an amine (R⁴—NH₂) with or without a base (e.g., NaH, Pyridine and the like) in a solvent (e.g., THF, Pyridine, MeCN and the like) to provide intermediates of formula 10. Pyrrole of formula 11 may be obtained by deprotection of compound 10 following procedures known to the skilled in the art (e.g., treatment with a base (e.g., Na₂CO₃ or LiOH and the like) if PG=Ts or in presence of an acid (e.g., HCl, TFA and the like) if PG=Boc). 2-Bromo-pyrrole of formula 12 may be obtained by bromination of compound 11 in presence of a brominating agent (e.g., NBS and the like) in a polar solvent (e.g., DMF and the like) following procedures known to the skilled in the art. 2-Bromo-pyrrole of formula 12 may be coupled with a boronic acid, boronic ester or a tin derivative (commercially available or synthesized by procedures known to the person skilled in the art) in presence of a palladium catalyst (e.g., Pd(PPh₃)₄, Pd(dppf)Cl₂ and the like) and a salt (e.g., KF, K₃PO₄, Na₂CO₃ and the like) in a solvent (e.g., DMF, toluene, dioxane, water, and the like) at a temperature ranging from 0 to 100° C. to provide the desired compound of formula 4. Alternatively, the bromo derivatives 12 may be converted in a boronic ester of general formula 13, via a Miyaura Borylation Reaction (For an article of such methods, see e.g., T. Ishiyama, M. Murata, N. Miyaura, J. Org. Chem., 1995, 60, 7508-7510). The desired compound of general formula 4 may be obtained via a Suzuki coupling between the boronic ester 13 and R¹—X (commercially available or synthesized by procedures known to the person skilled in the art).

Alternatively, 2-Bromo-pyrrole intermediates of formula 12 may be converted in a protected pyrrole of formula 12a, following procedures known to the person skilled in the art (e.g., treatment with TsCl, Boc₂O, (i-Pr)₃SiCl, in the presence of a base (e.g., NaH, Et₃N, DMAP and the like) and in solvent (e.g., THF, DCM, MeCN and the like)). Halogenated pyrroles of formula 12a may be then coupled with a boronic acid, boronic ester or a tin derivative (commercially available or synthesized by procedures known to the person skilled in the art) in presence of a palladium catalyst (e.g., Pd(PPh₃)₄, Pd(dppf)Cl₂ and the like) and a salt (e.g., KF, K₃PO₄, Na₂CO₃ and the like) in a solvent (e.g., DMF, toluene, dioxane, water, and the like) at a temperature ranging from 0 to 100° C. to provide the desired compounds of formula 12b. Pyrroles of formula 4 may be obtained by deprotection of a compound 12b following procedures known to the skilled in the art (e.g., treatment with a base (e.g., Na₂CO₃ or LiOH and the like) if PG=Ts or in presence of an acid (e.g., HCl, TFA and the like) if PG=Boc).

In another embodiment, compounds of the present invention may also be synthesized according to the general procedure outlined in Scheme 3.

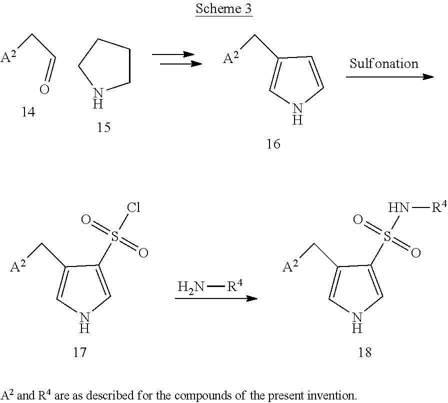

Pyrrole of general formula 16 can be obtained in 2 steps synthesis from the condensation between aldehydes 14, commercially available or synthesized by procedures known to the skilled in the art, and the pyrrolidine 15 as described in Org. Lett. 2015, 17, 3762-3765 (DOI: 10.1021/acs.orglett.5b01744). Pyrrole of formula 17 may be obtained from compound of general formula 16 using a sulfonyl-chlorinating agent (e.g., Chlorosulfonic acid and the like) in a polar solvent (e.g., MeCN and the like. Alternatively, the compound of general formula 17 may be obtained from compound of general formula 16 using a sulfonating agent (e.g., SO₃, Py·SO₃ and the like) in a polar solvent (e.g., MeCN, DCM and the like followed by a subsequent reaction with a chlorination reagent (e.g., POCl₃, thionyl chloride, oxalyl chloride and the like) in a polar solvent (e.g., MeCN, DCM and the like). Compounds of interest having a general formula 18 may be obtained via the condensation of sulfonyl chloride derivative 17 with an amine (R⁴—NH₂) in presence of a base (e.g., NaH, Pyridine and the like) in a solvent (e.g., THF, Pyridine, MeCN and the like).

In another embodiment, compounds of the present invention may also be synthesized according to the general procedure outlined in Scheme 4.

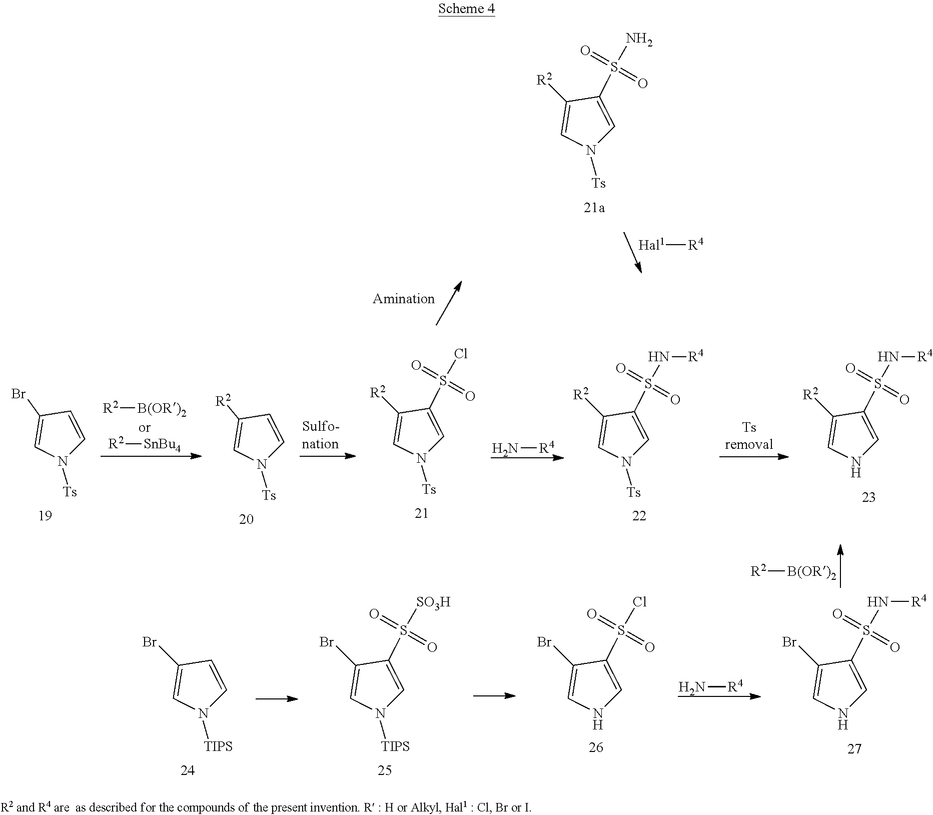

3-Bromo-1-tosyl-1H-pyrrole 19, commercially available, may be coupled with a boronic acid, boronic ester or a tin derivative (commercially available or synthesized by procedures known to the person skilled in the art) in presence of a palladium catalyst (e.g., Pd(PPh₃)₄, Pd(dppf)Cl₂ and the like) and a salt (e.g., KF, K₃PO₄, Na₂CO₃ and the like) in a solvent (e.g., DMF, toluene, dioxane, water, and the like) at a temperature ranging from 0 to 100° C. to provide intermediates of formula 20. Pyrrole of formula 21 may be directly obtained from compound of general formula 20 using a sulfonyl-chlorinating agent (e.g., Chlorosulfonic acid and the like) in a polar solvent (e.g., MeCN and the like). Alternatively, the compound of general formula 21 may be obtained from compound of general formula 20 using a sulfonating agent (e.g., SO₃, Py·SO₃ and the like) in a polar solvent (e.g., MeCN, DCM and the like) followed by a subsequent reaction with a chlorination reagent (e.g., POCl₃, thionyl chloride, oxalyl chloride and the like) in a polar solvent (e.g., MeCN, DCM and the like). Sulfonyl chloride derivative 21 may be condensed with an amine (R⁴—NH₂) with or without a base (e.g., NaH, Pyridine and the like) in a solvent (e.g., THF, Pyridine, MeCN and the like) to provide intermediates of formula 22. Alternatively, the compounds of general formula 21 may be converted in the intermediate of formula 21a by treatment with aq. NH₃ in a solvent (e.g., THF and the like), followed by a Buchwald-Hartwig-type coupling reaction with a halogenated reagent Hal¹-R⁴ (commercially available or synthesized by procedures known to the person skilled in the art) in the presence of a copper catalyst (e.g., CuI and the like), a ligand (e.g., trans-N,N-dimethylcyclohexane-1,2-diamine and the like), a base (e.g., K₂CO₃ and the like), in a solvent (e.g., MeCN, DCM and the like) to provide intermediates of formula 22. Compounds of interest having a general formula 23 may be obtained by deprotection of compound 22 by a treatment with a base (e.g., Na₂CO₃ or LiOH and the like) in a protic solvent (e.g., water, MeOH and the like). Alternatively, 3-bromo-1-(triisopropylsilyl)-1H-pyrrole 24, commercially available, may be reacted with a sulfonating reagent (e.g., CISO₃H, Py·SO₃ and the like) in a polar solvent (e.g., MeCN, DCM and the like) to afford the intermediate of general formula 25. Derivatives of formula 26 may be obtained from compound of general formula 25 using a -chlorinating agent (e.g., Oxalyl chloride, POCl₃ and the like) in a solvent (e.g., DCM and the like). Sulfonyl chloride derivative 26 may be condensed with an amine (R⁴—NH₂) in presence or absence of a base (e.g., NaH, Pyridine and the like) in a solvent (e.g., THF, Pyridine, MeCN and the like) to provide the compound of general structure 27. Compounds of interest having a general formula 23 may be obtained via a Suzuki coupling between the boronic acid, R²—B(OR′)₂ (commercially available or synthesized by procedures known to the person skilled in the art) and 3-bromo-pyrrole of general formula 27.

In another embodiment, compounds of the present invention may also be synthesized according to the general procedure outlined in Scheme 5.

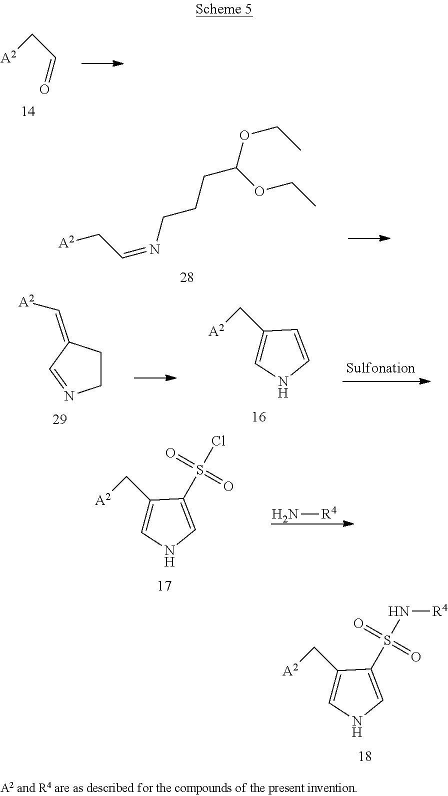

Compounds of formula 28 can be obtained from the condensation of aldehydes of formula 14, commercially available or synthesized by procedures known to the skilled in the art, with 4,4-diethoxy-butylamine, in a solvent (e.g., CHCl₃ and the like). Compounds of formula 29 can be obtained by the treatment of an intermediate 28 with an acid (e.g., TsOH and the like) in a solvent (e.g., xylene, toluene and the like). Intermediates 29 can be converted into intermediates of general formula 16, by treatment with a base (e.g., t-BuOK and the like) in a solvent (e.g., DMSO and the like). Compounds of general formula 17 and 18 can then be obtained from compounds of general formula 16, as per described in Scheme 3.

In another embodiment, compounds of the present invention may also be synthesized according to the general procedure outlined in Scheme 6.

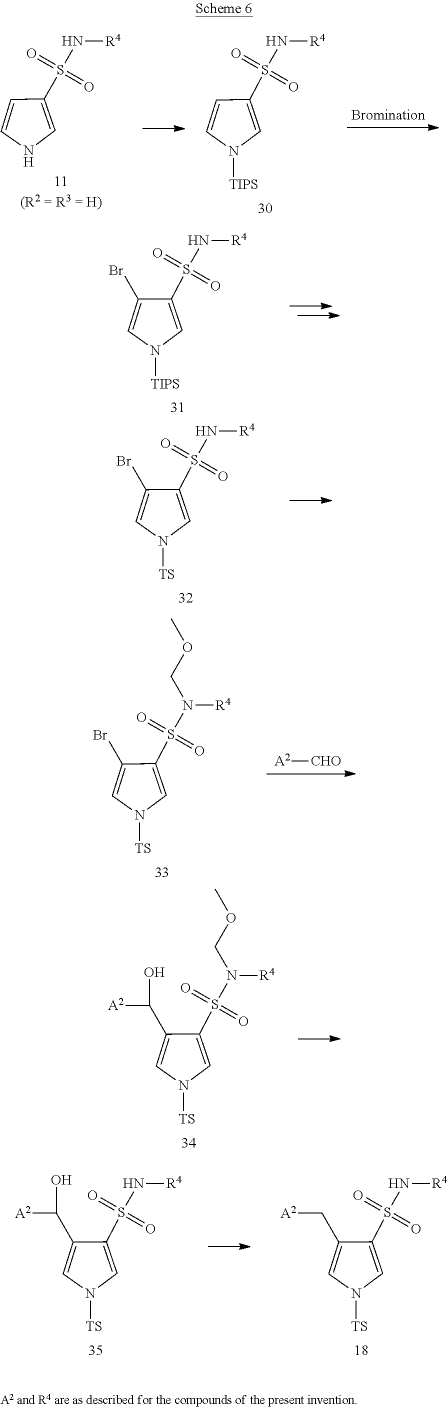

Compounds of formula 11 (wherein R²═R³═H) can be commercially available or synthesized according to scheme 2 and scheme 2a. They may be converted in TIPS-protected pyrroles of formula 30, following procedures known to the person skilled in the art (e.g., treatment with (i-Pr)₃SiCl, in the presence of a base (e.g., NaH, Et₃N, DMAP and the like) and in solvent (e.g., THF, DCM, MeCN and the like)). Compounds 31 may be obtained by bromination of compounds of formula 30 in presence of a brominating agent (e.g., NBS and the like) in a polar solvent (e.g., DMF, THF and the like) following procedures known to the skilled in the art. Compounds of formula 32 may be obtained by successive deprotection of intermediates 31 using procedures known to the person skilled in the art (e.g., treatment with fluorinated agent (e.g., TBAF and the like) in a solvent (e.g., THF and the like)), followed by protection with a Tosyl group using procedures known to the person skilled in the art (e.g., treatment with TsCl in a solvent (e.g., NaH, Et₃N, DMAP and the like) and in solvent (e.g., THF, DCM, MeCN and the like)). Intermediates of general formula 33, may be prepared by treating intermediates 32 with chloromethyl methyl ether in the presence of a base (e.g., DIPEA and the like), in a solvent (e.g., DCM and the like). Compounds 34 may be prepared by reacting intermediates 33 with an organometallic reagent (e.g., iPrMgCl and the like) in a solvent (e.g., THF, DME and the like), followed by the addition of an aldehyde A²—CHO (commercially available or synthesized by procedures known to the person skilled in the art). Intermediates 34 may be deprotected into intermediates 35 following procedures known to the skilled in the art (e.g., treatment with a base (e.g., Na₂CO₃ or LiOH and the like)). Compounds of interest having a general formula 18 may be obtained by treating intermediates 35 with a reducing reagent (e.g., Et₃SiH and the like) in a solvent (e.g., DCE and the like).

In another embodiment, compounds of the present invention may also be synthesized according to the general procedure outlined in Scheme 7.

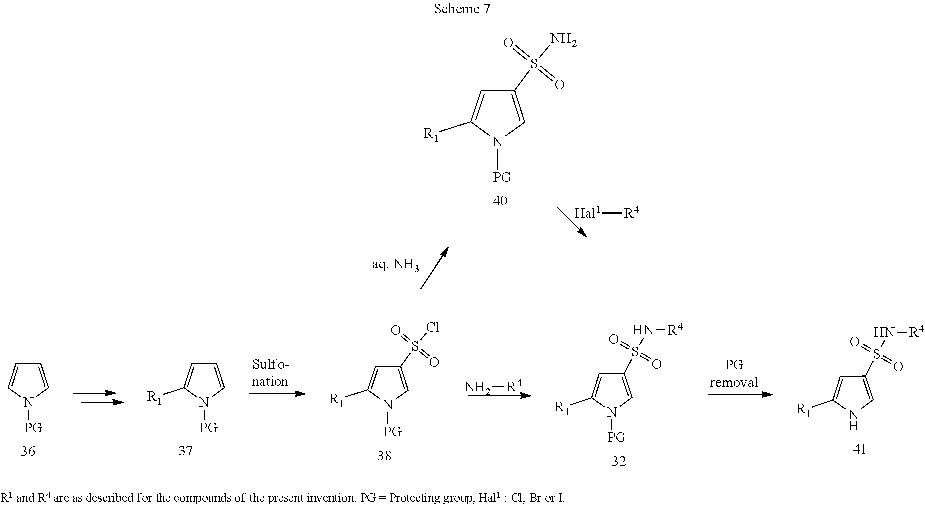

Compounds of formula 36, commercially available, may be treated with a strong base (e.g., t-BuLi, i-PrMgCl and the like) in a solvent (e.g., THF and the like) followed by the addition of an appropriate ketone (commercially available or synthesized by procedures known to the person skilled in the art) and further dehydrated by a reducing agent (e.g., Et₃SiH and the like) in the presence of an acid (e.g., TFA and the like) in a solvent (e.g., DCM and the like) to afford intermediates of formula 37. Pyrroles of formula 38 may be directly obtained from compound of general formula 37 using a sulfonyl-chlorinating agent (e.g., Chlorosulfonic acid and the like) in a polar solvent (e.g., MeCN and the like) at a temperature ranging from 0 to 120° C. Alternatively, the compound of general formula 38 may be obtained from compound of general formula 37 using a sulfonating agent (e.g., SO₃, Py·SO₃ and the like) in a polar solvent (e.g., MeCN, DCM and the like) at a temperature ranging from 0 to 120° C. followed by a subsequent reaction with a chlorination reagent (e.g., POCl₃, thionyl chloride, oxalyl chloride and the like) in a polar solvent (e.g., MeCN, DCM and the like) at a temperature ranging from 0 to 120° C. Sulfonyl chloride derivatives 38 may be condensed with an amine (R⁴—NH₂) with or without a base (e.g., NaH, Pyridine and the like) in a solvent (e.g., THF, Pyridine, MeCN and the like) to afford compounds of generic formula 32. Alternatively, sulfonamide intermediates 32 may be prepared by condensation of sulfonyl chloride derivatives 38 with aq. NH₃ in a solvent (e.g., THF and the like) followed by a subsequent coupling type reaction of intermediates of general formula 40 with an halogenated compound of formula Hal¹-R⁴ in the presence of a catalyst (e.g., CuI and the like), a ligand (e.g., trans-N,N-dimethylcyclohexane-1,2-diamine and the like), a base (e.g., K₂CO₃ and the like) and a polar solvent (e.g., MeCN and the like). Compounds of interest of generic formula 41 can be prepared by deprotection of intermediates 32 following procedures known to the skilled in the art (e.g., treatment with a base such as Na₂CO₃ if PG=Ts or in presence of an acid (e.g., HCl, TFA and the like) if PG=Boc).

The general schemes depicted above should be considered as non-limiting examples. It will be understood that compounds of the invention may be obtained through other methods, which are known to people skilled in the art.

The following examples are provided for the purpose of illustrating the present invention and by no means should be interpreted to limit the scope of the present invention.

### EXAMPLES

TABLE 1
Structures of example compounds of the
invention and their respective codes

| Structure | CODE |
|---|---|
| Cpd 001 | 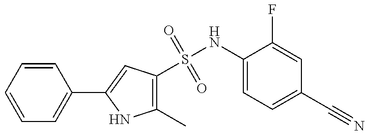 |
| Cpd 002 | 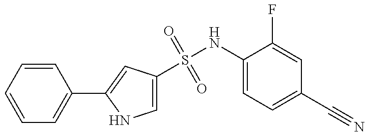 |
| Cpd 003 | 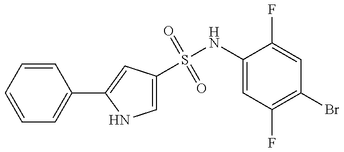 |
| Cpd 004 | 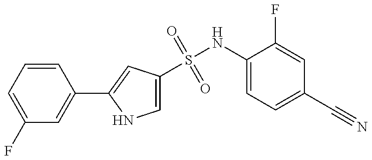 |
| Cpd 005 | 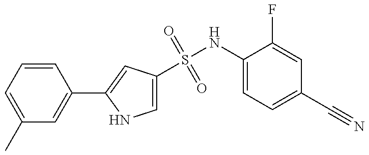 |
| Cpd 006 | 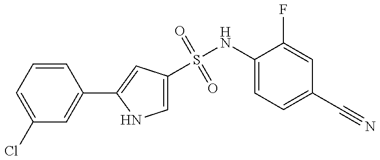 |
| Cpd 007 | 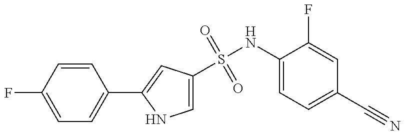 |
| Cpd 008 | 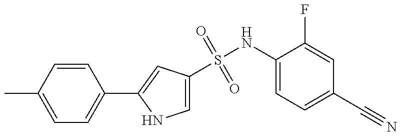 |
| Cpd 009 | 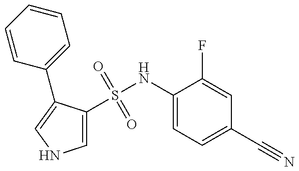 |
| Cpd 010 | 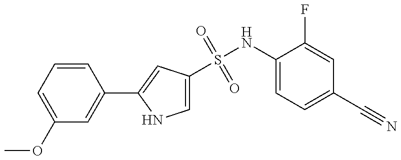 |
| Cpd 011 | 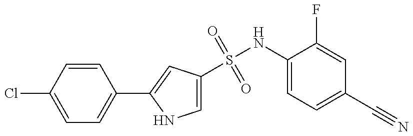 |
| Cpd 012 | 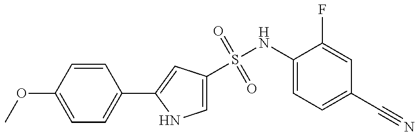 |
| Cpd 013 | 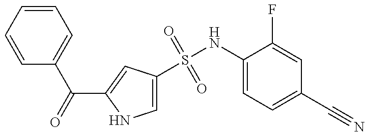 |
| Cpd 014 | 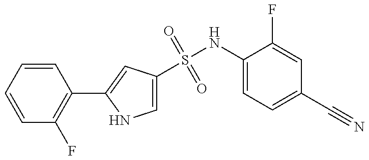 |
| Cpd 015 | 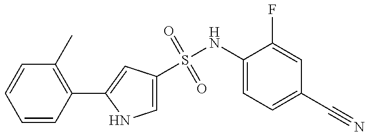 |
| Cpd 016 | 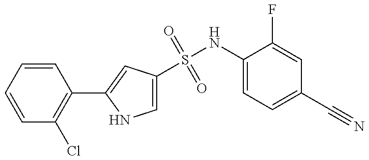 |
| Cpd 017 | 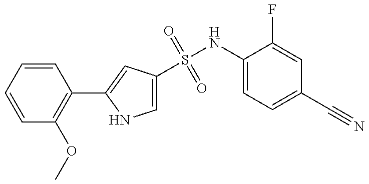 |
| Cpd 018 | 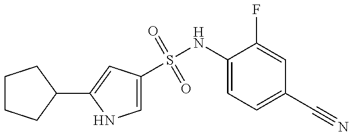 |
| Cpd 019 | 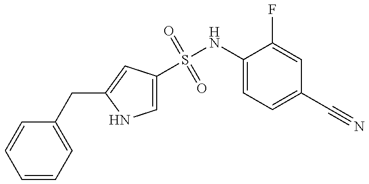 |
| Cpd 020 | 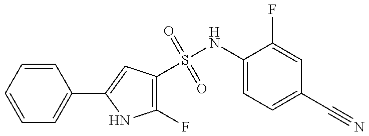 |
| Cpd 021 | 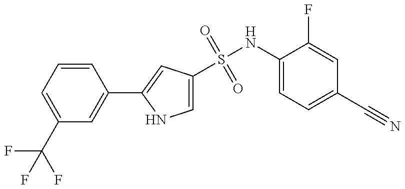 |
| Cpd 022 | 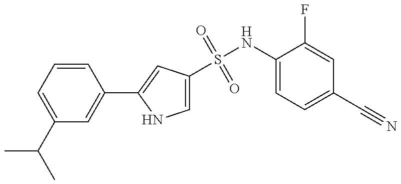 |
| Cpd 023 | 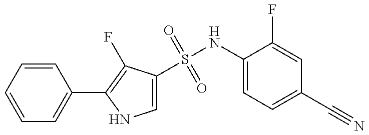 |
| Cpd 024 | 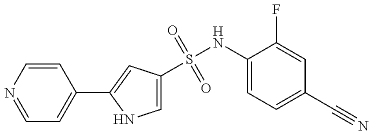 |
| Cpd 025 | 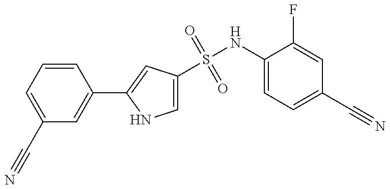 |
| Cpd 026 | 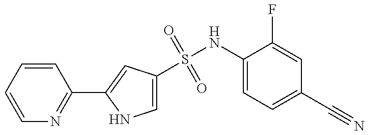 |
| Cpd 027 | 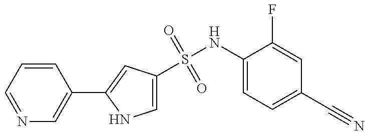 |
| Cpd 028 | 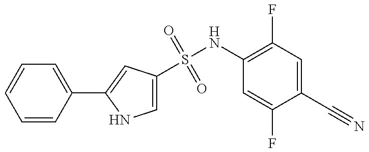 |
| Cpd 029 | 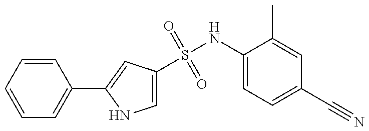 |
| Cpd 030 | 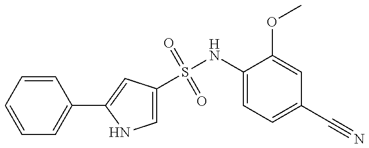 |
| Cpd 031 | 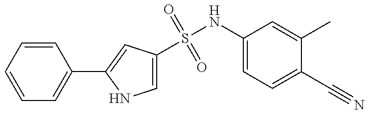 |
| Cpd 032 | 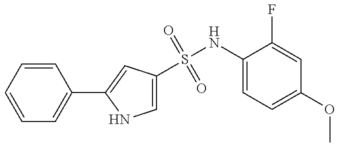 |
| Cpd 033 | 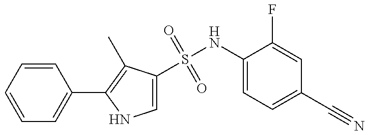 |
| Cpd 034 | 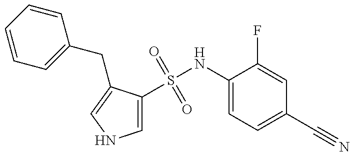 |
| Cpd 035 | 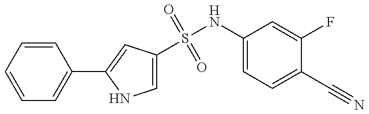 |
| Cpd 036 | 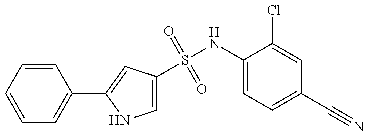 |
| Cpd 037 | 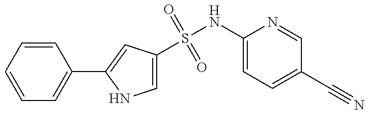 |
| Cpd 038 | 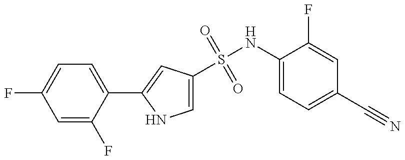 |
| Cpd 039 | 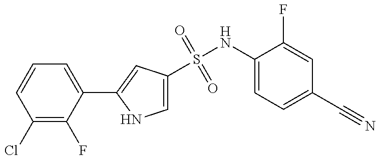 |
| Cpd 040 | 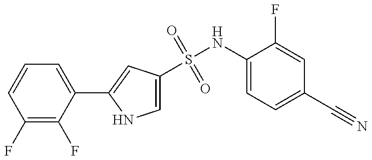 |
| Cpd 041 | 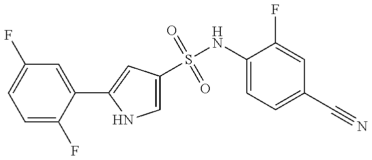 |
| Cpd 042 | 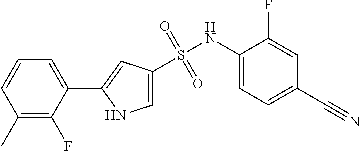 |
| Cpd 043 | 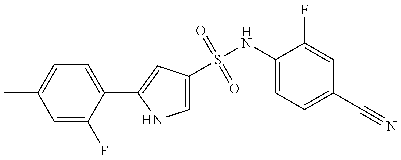 |
| Cpd 044 | 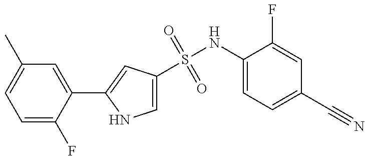 |
| Cpd 045 | 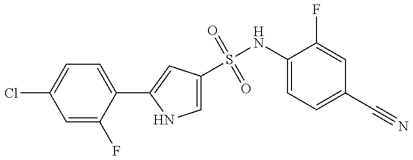 |
| Cpd 046 | 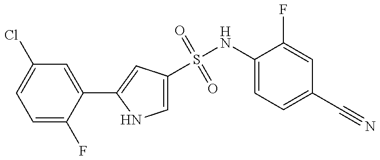 |
| Cpd 047 | 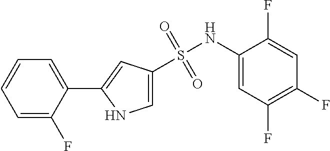 |
| Cpd 048 | 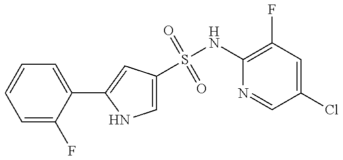 |
| Cpd 049 | 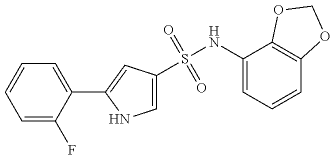 |
| Cpd 050 | 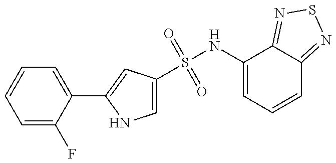 |
| Cpd 051 | 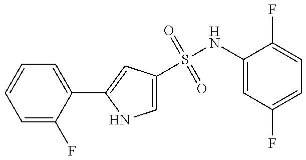 |
| Cpd 052 | 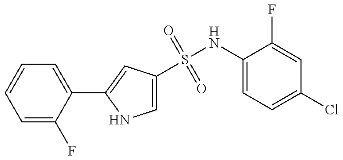 |
| Cpd 053 | 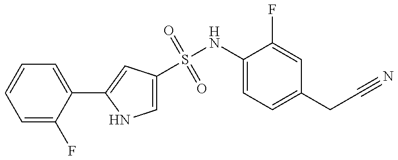 |
| Cpd 054 | 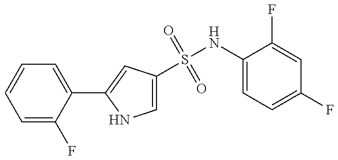 |
| Cpd 055 | 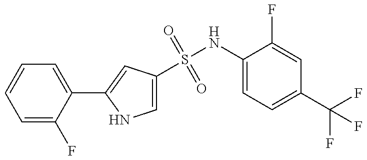 |
| Cpd 056 | 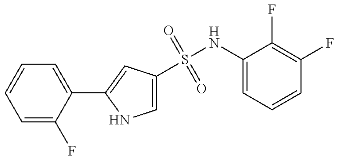 |
| Cpd 057 | 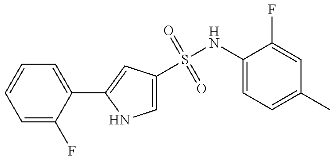 |
| Cpd 058 | 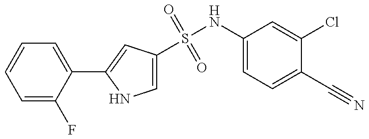 |
| Cpd 059 | 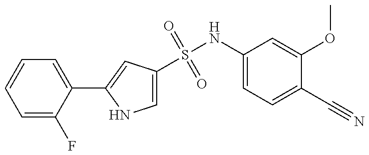 |
| Cpd 060 | 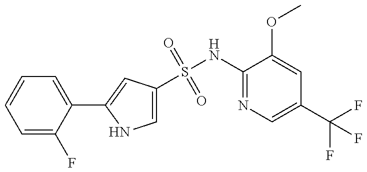 |
| Cpd 061 | 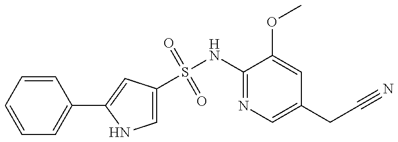 |
| Cpd 062 | 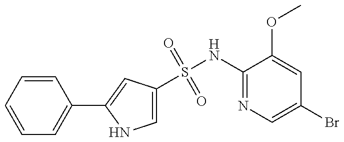 |
| Cpd 063 | 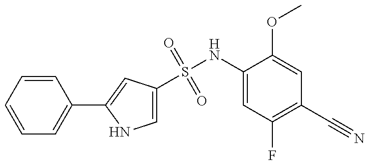 |
| Cpd 064 | 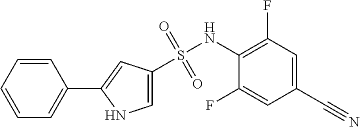 |
| Cpd 065 | 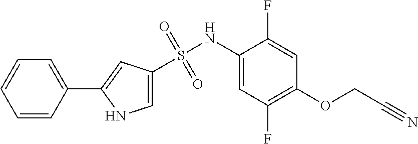 |
| Cpd 066 | 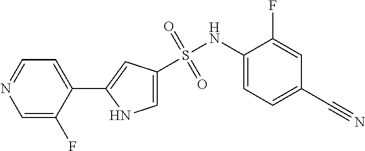 |
| Cpd 067 | 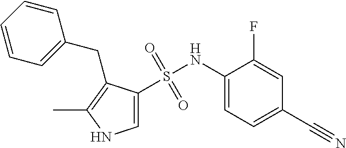 |
| Cpd 068 | 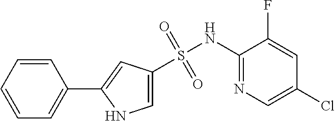 |
| Cpd 069 | 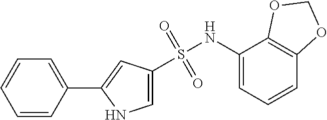 |
| Cpd 070 | 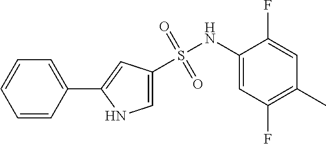 |
| Cpd 071 | 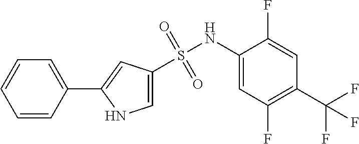 |
| Cpd 072 | 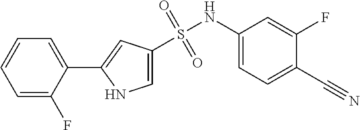 |
| Cpd 073 | 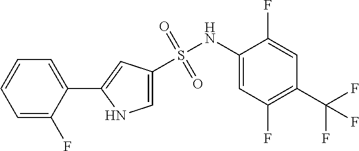 |
| Cpd 074 | 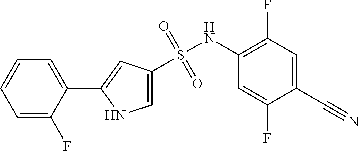 |
| Cpd 075 | 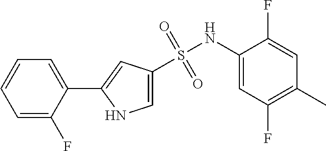 |
| Cpd 076 | 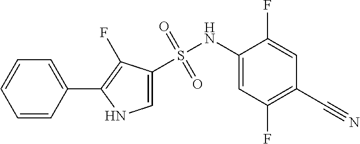 |
| Cpd 077 | 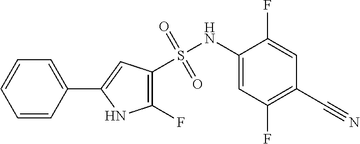 |
| Cpd 078 | 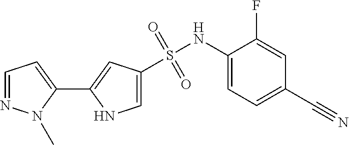 |
| Cpd 079 | 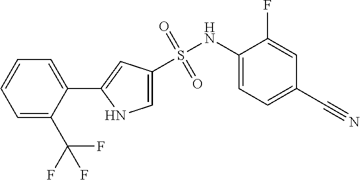 |
| Cpd 080 | 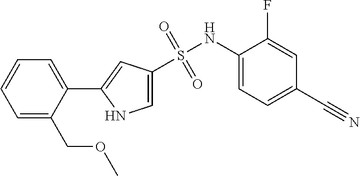 |
| Cpd 081 | 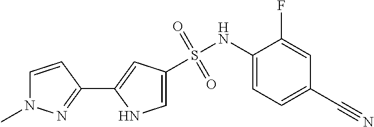 |
| Cpd 082 | 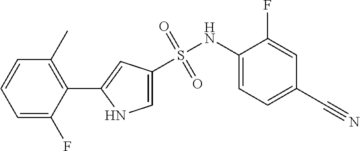 |
| Cpd 083 | 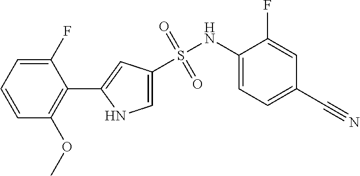 |
| Cpd 084 | 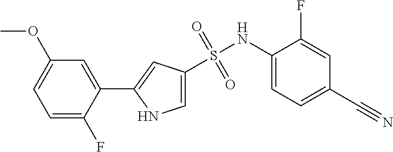 |
| Cpd 085 | 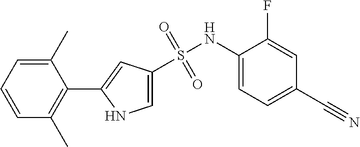 |
| Cpd 086 | 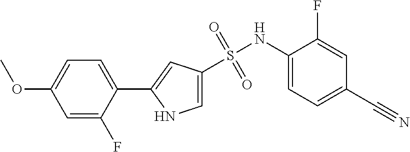 |
| Cpd 087 | 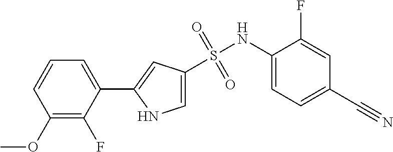 |
| Cpd 088 | 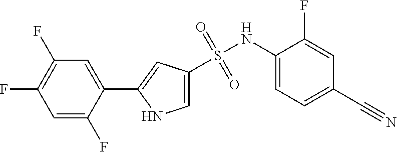 |
| Cpd 089 | 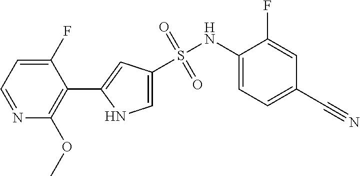 |
| Cpd 090 | 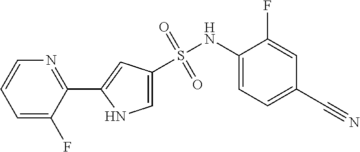 |
| Cpd 091 | 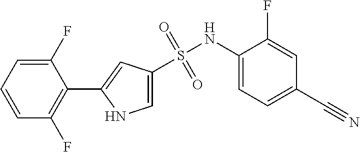 |
| Cpd 093 | 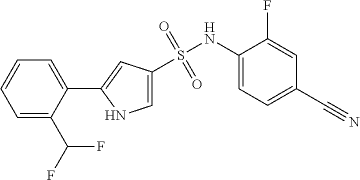 |
| Cpd 094 | 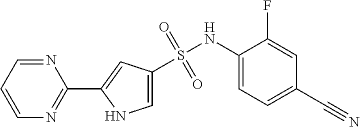 |
| Cpd 095 | 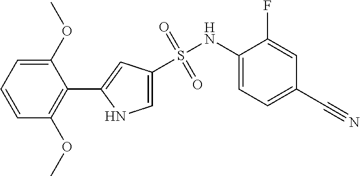 |
| Cpd 096 | 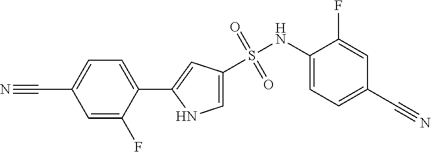 |
| Cpd 097 | 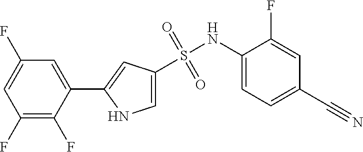 |
| Cpd 098 | 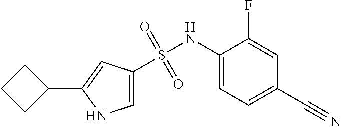 |
| Cpd 099 | 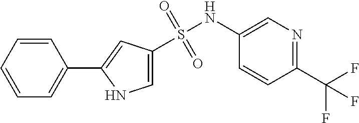 |
| Cpd 100 | 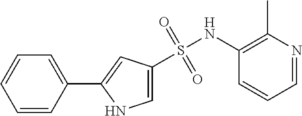 |
| Cpd 101 | 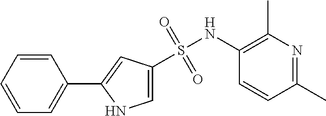 |
| Cpd 102 | 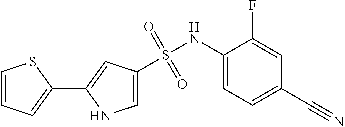 |
| Cpd 103 | 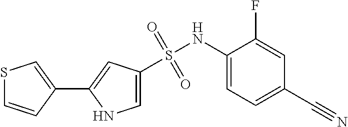 |
| Cpd 104 | 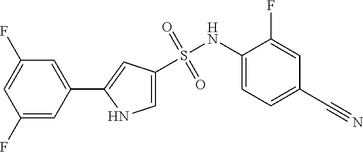 |
| Cpd 105 | 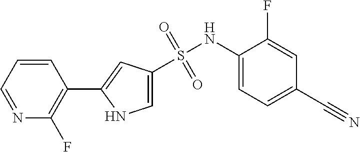 |
| Cpd 106 | 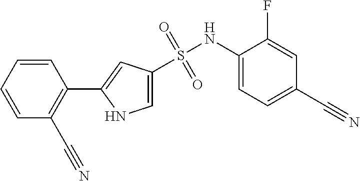 |
| Cpd 107 | 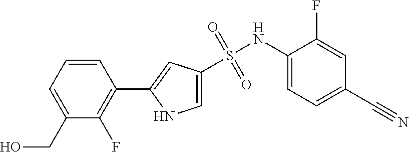 |
| Cpd 108 | 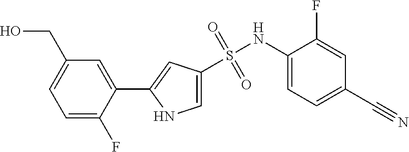 |
| Cpd 109 | 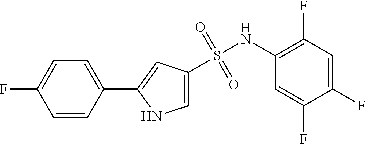 |
| Cpd 110 | 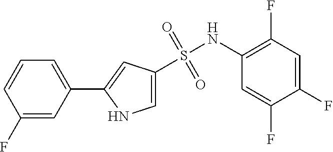 |
| Cpd 111 | 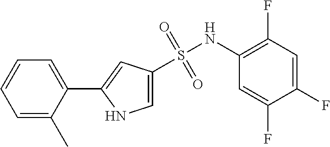 |
| Cpd 112 | 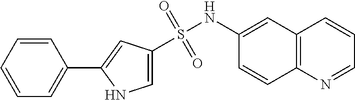 |
| Cpd 113 | 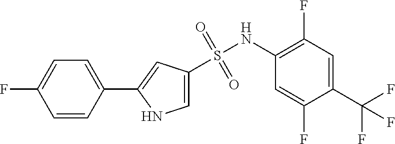 |
| Cpd 114 | 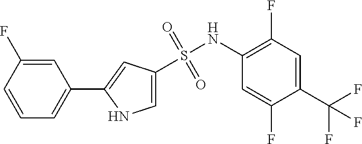 |
| Cpd 115 | 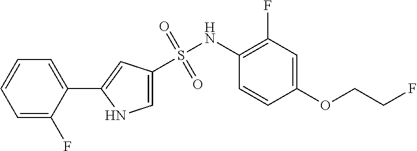 |
| Cpd 116 | 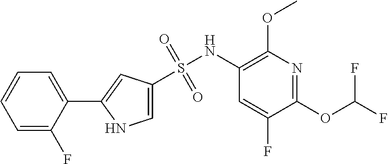 |
| Cpd 117 | 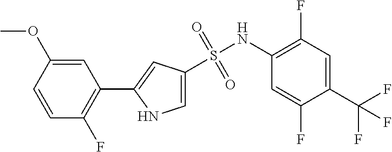 |
| Cpd 118 | 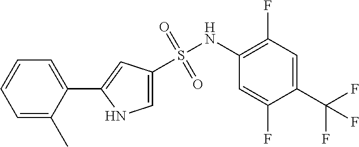 |
| Cpd 119 | 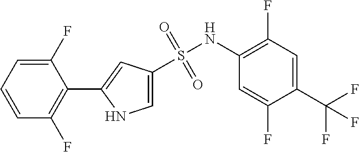 |
| Cpd 120 | 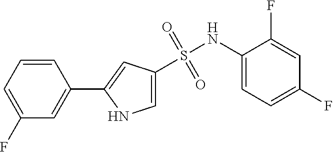 |
| Cpd 121 | 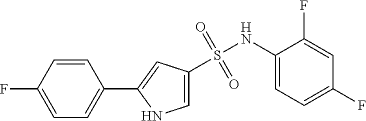 |
| Cpd 122 | 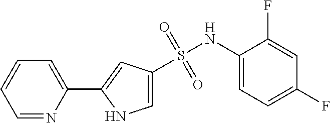 |
| Cpd 123 | 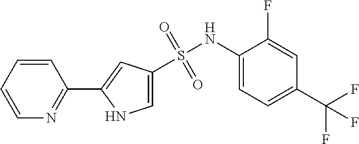 |
| Cpd 124 | 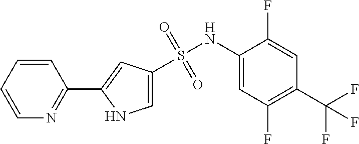 |
| Cpd 125 | 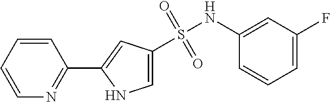 |
| Cpd 126 | 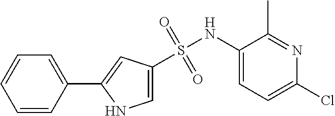 |
| Cpd 127 | 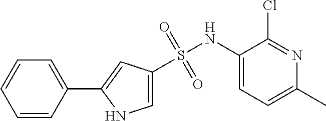 |
| Cpd 128 | 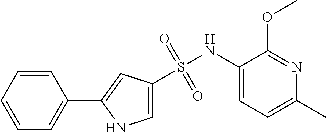 |
| Cpd 129 | 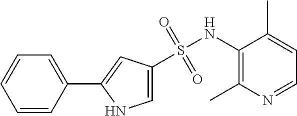 |
| Cpd 130 | 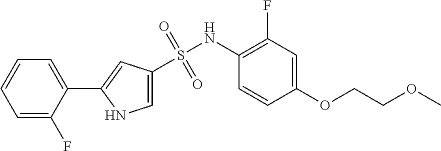 |
| Cpd 131 | 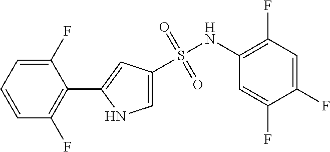 |
| Cpd 132 | 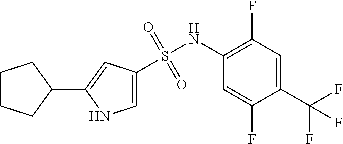 |
| Cpd 133 | 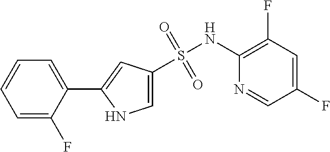 |
| Cpd 134 | 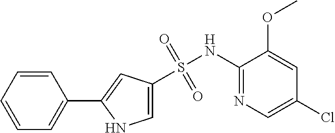 |
| Cpd 135 | 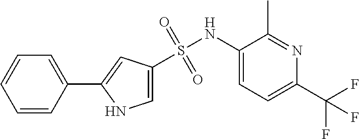 |
| Cpd 136 | 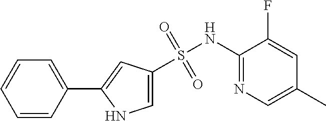 |
| Cpd 137 | 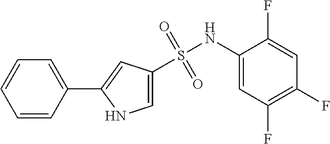 |
| Cpd 138 | 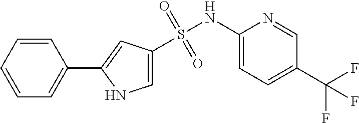 |
| Cpd 139 | 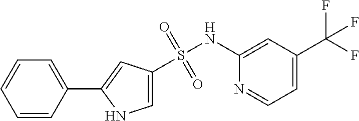 |
| Cpd 140 | 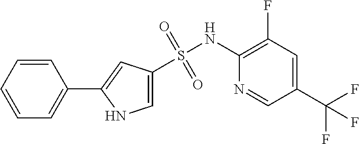 |
| Cpd 141 | 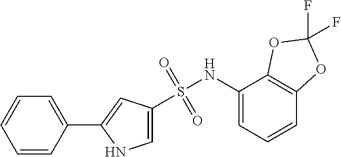 |
| Cpd 142 | 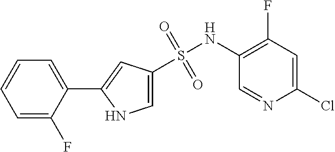 |
| Cpd 143 | 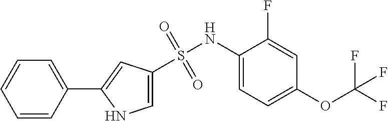 |
| Cpd 144 | 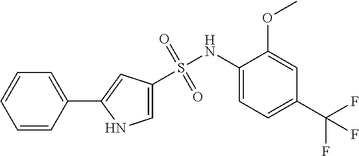 |
| Cpd 145 | 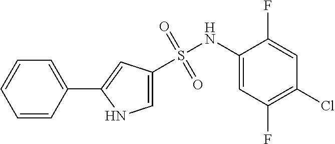 |
| Cpd 146 | 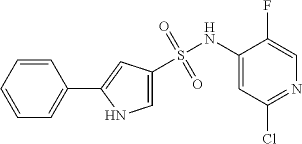 |
| Cpd 147 | 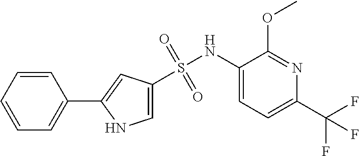 |
| Cpd 148 | 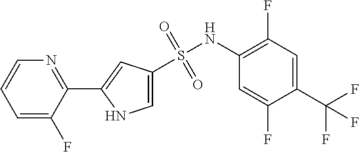 |
| Cpd 149 | 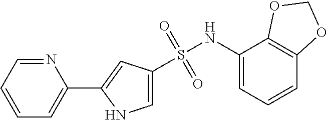 |
| Cpd 150 | 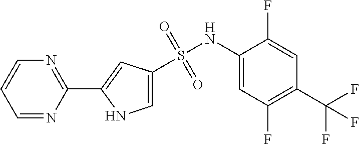 |
| Cpd 151 | 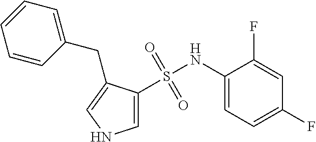 |
| Cpd 152 | 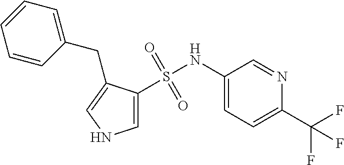 |
| Cpd 153 | 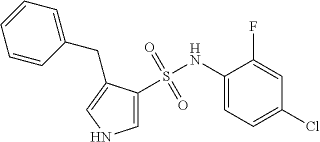 |
| Cpd 154 | 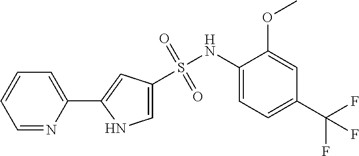 |
| Cpd 155 | 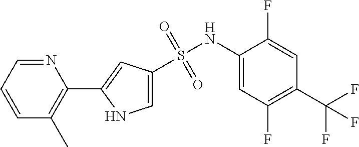 |
| Cpd 156 | 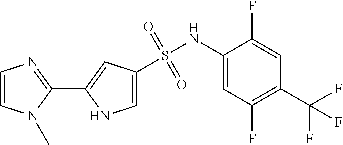 |
| Cpd 157 | 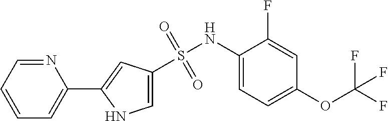 |
| Cpd 158 | 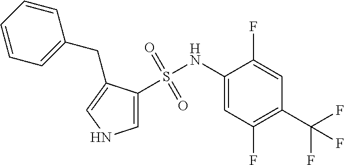 |
| Cpd 159 | 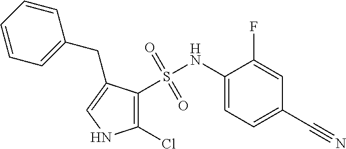 |
| Cpd 160 | 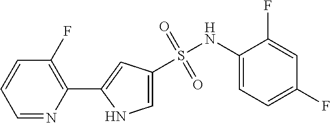 |
| Cpd 161 | 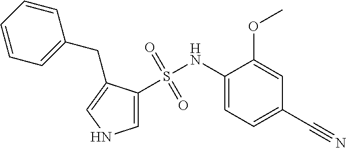 |
| Cpd 162 | 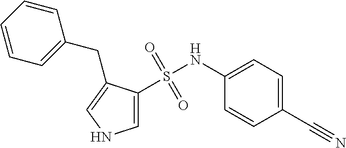 |
| Cpd 163 | 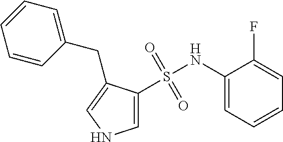 |
| Cpd 164 | 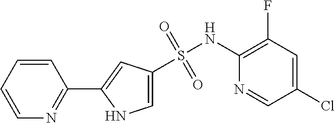 |
| Cpd 165 | 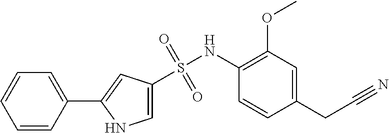 |
| Cpd 166 | 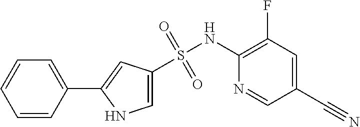 |
| Cpd 167 | 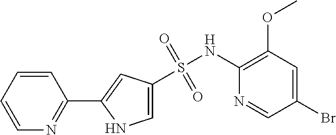 |
| Cpd 168 | 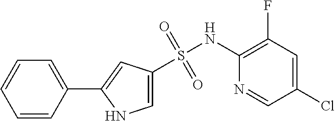 |
| Cpd 169 | 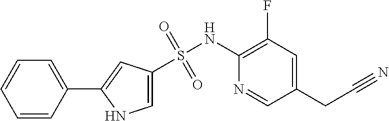 |
| Cpd 170 | 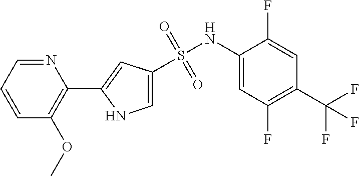 |
| Cpd 171 | 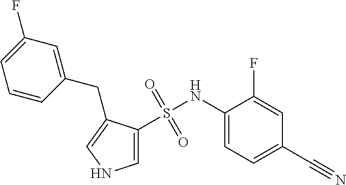 |
| Cpd 172 | 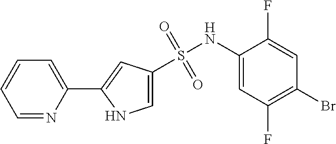 |
| Cpd 173 | 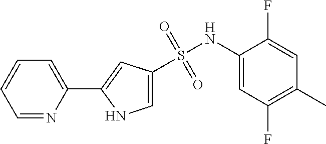 |
| Cpd 174 | 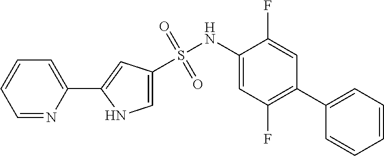 |
| Cpd 175 | 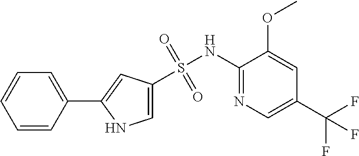 |
| Cpd 176 | 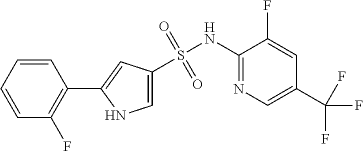 |
| Cpd 177 | 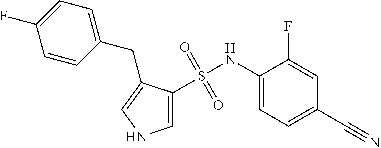 |
| Cpd 178 | 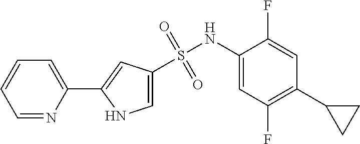 |
| Cpd 179 | 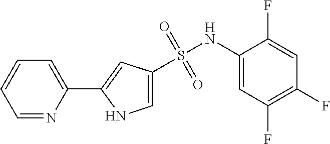 |
| Cpd 180 | 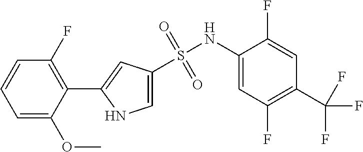 |
| Cpd 181 | 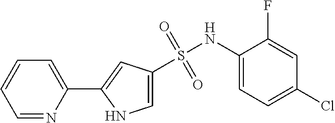 |
| Cpd 182 | 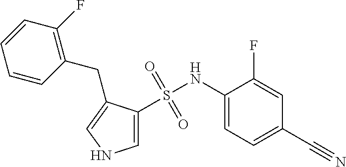 |
| Cpd 183 | 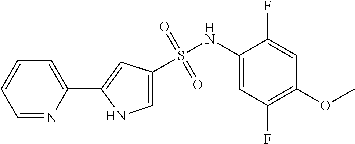 |
| Cpd 184 | 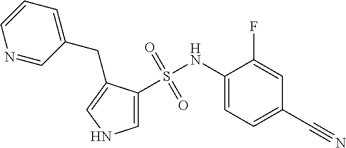 |
| Cpd 185 | 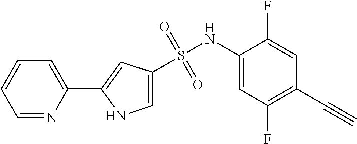 |
| Cpd 186 | 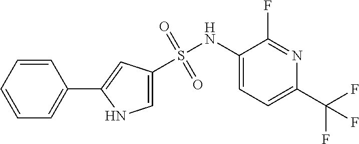 |
| Cpd 187 | 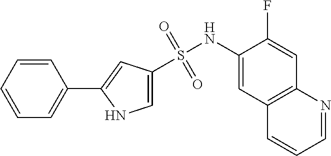 |
| Cpd 188 | 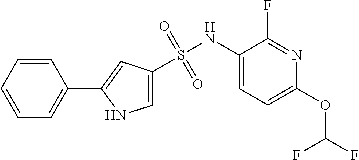 |
| Cpd 189 | 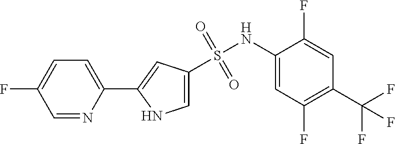 |
| Cpd 190 | 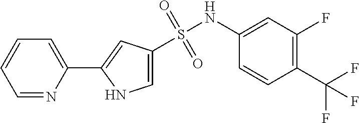 |
| Cpd 191 | 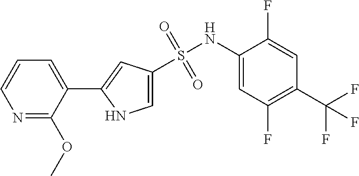 |
| Cpd 192 | 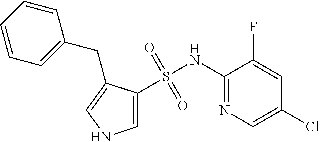 |
| Cpd 193 | 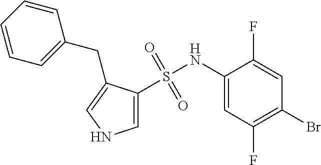 |
| Cpd 194 | 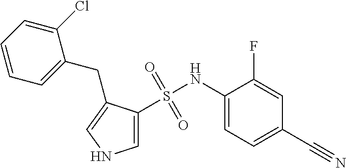 |
| Cpd 195 | 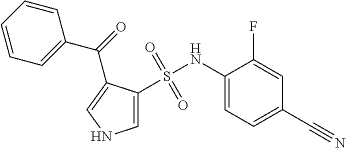 |
| Cpd 196 | 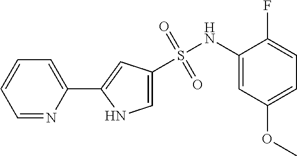 |
| Cpd 197 | 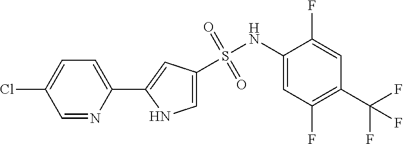 |
| Cpd 198 | 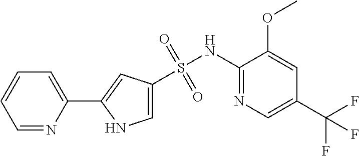 |
| Cpd 199 | 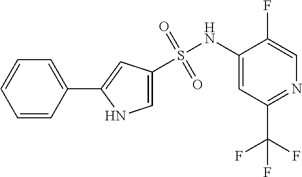 |
| Cpd 200 | 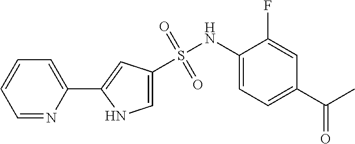 |
| Cpd 201 | 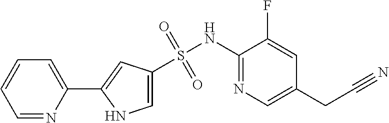 |
| Cpd 202 | 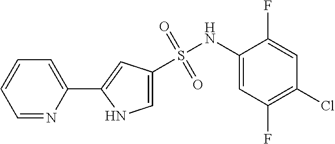 |
| Cpd 203 | 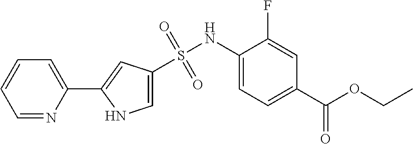 |
| Cpd 204 | 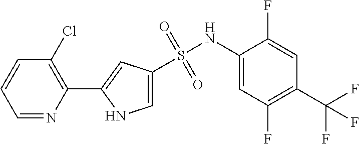 |
| Cpd 205 | 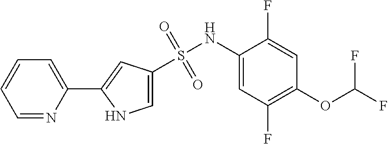 |
| Cpd 206 | 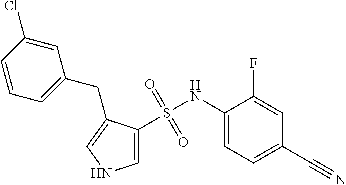 |
| Cpd 207 | 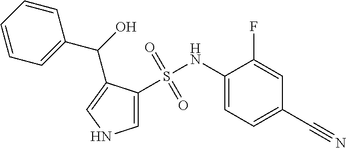 |
| Cpd 208 | 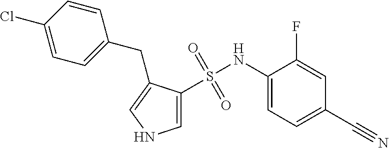 |
| Cpd 209 | 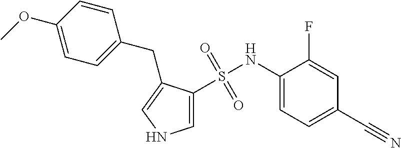 |
| Cpd 210 | 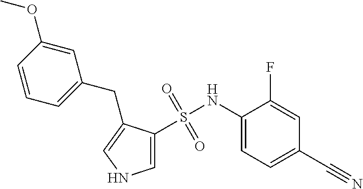 |
| Cpd 211 | 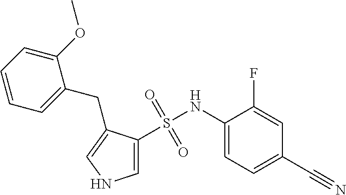 |
| Cpd 212 | 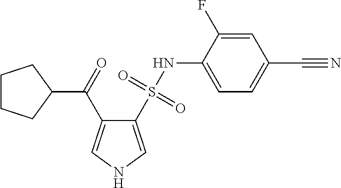 |
| Cpd 213 | 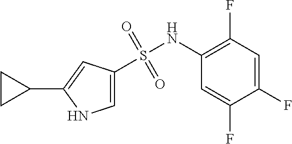 |
| Cpd 214 | 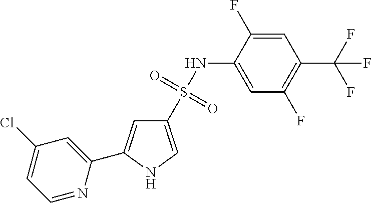 |
| Cpd 215 | 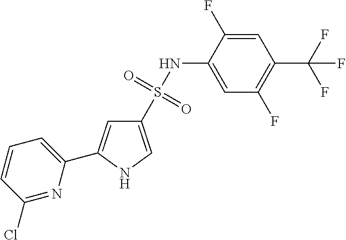 |
| Cpd 216 | 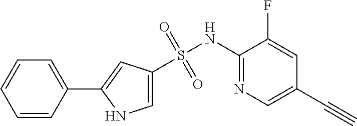 |
| Cpd 217 | 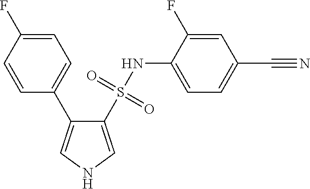 |
| Cpd 218 | 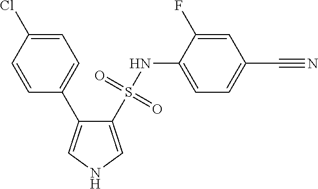 |
| Cpd 219 | 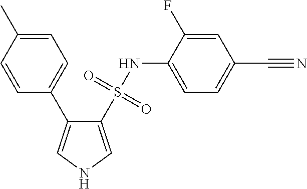 |
| Cpd 220 | 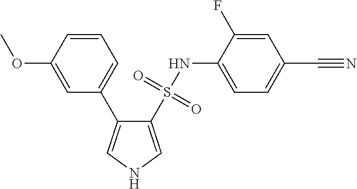 |
| Cpd 221 | 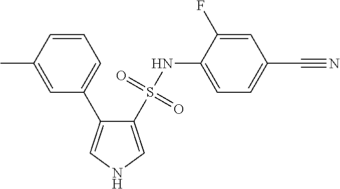 |
| Cpd 222 | 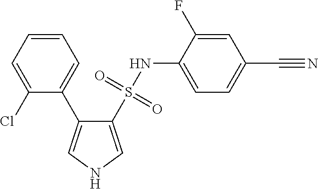 |
| Cpd 223 | 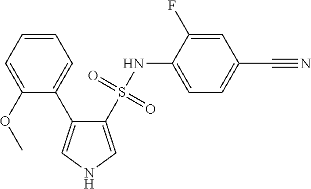 |
| Cpd 224 | 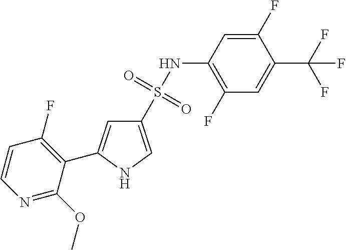 |
| Cpd 225 | 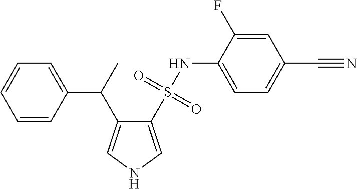 |
| Cpd 226 | 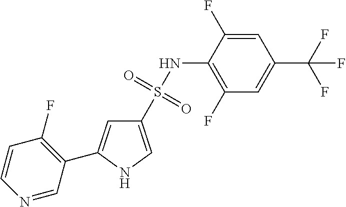 |
| Cpd 227 | 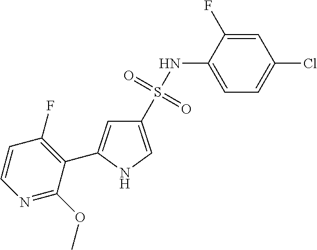 |
| Cpd 228 | 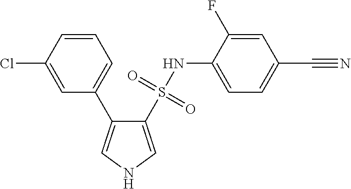 |
| Cpd 229 | 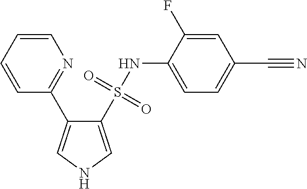 |
| Cpd 230 | 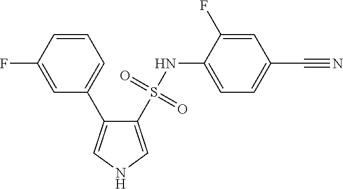 |
| Cpd 231 | 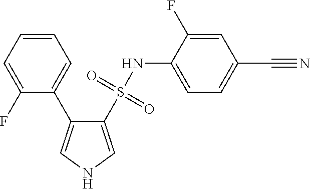 |
| Cpd 232 | 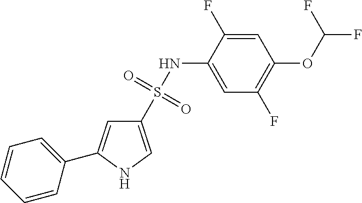 |
| Cpd 233 | 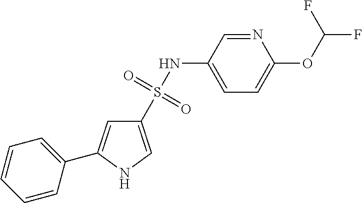 |
| Cpd 234 | 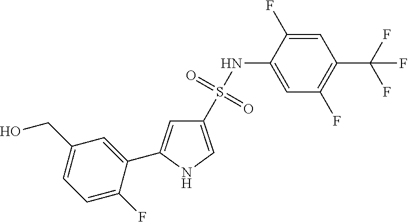 |
| Cpd 235 | 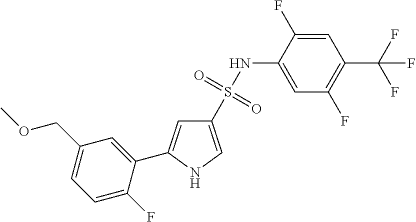 |
| Cpd 236 | 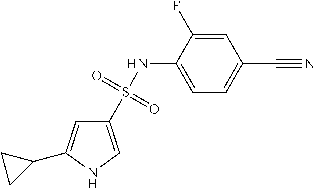 |
| Cpd 237 | 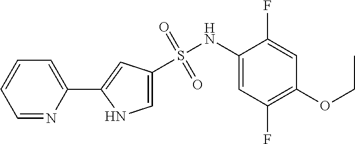 |
| Cpd 238 | 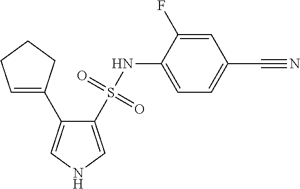 |
| Cpd 239 | 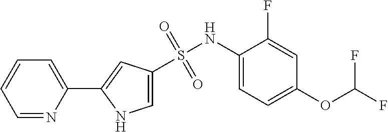 |
| Cpd 240 | 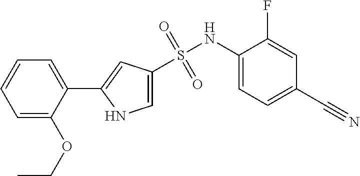 |
| Cpd 241 | 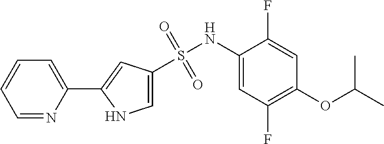 |
| Cpd 242 | 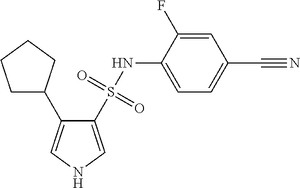 |
| Cpd 243 | 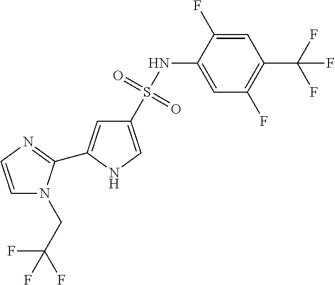 |
| Cpd 244 | 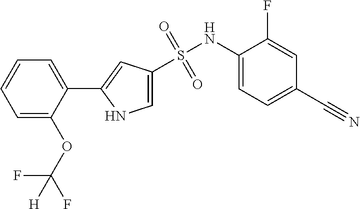 |
| Cpd 245 | 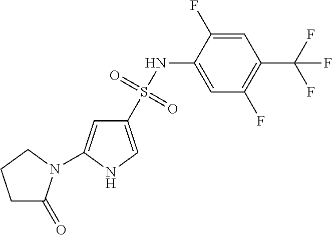 |
| Cpd 246 | 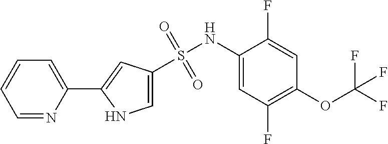 |
| Cpd 247 | 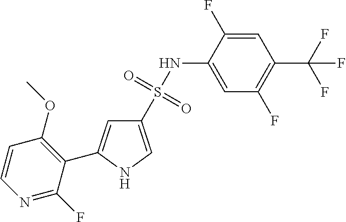 |
| Cpd 248 | 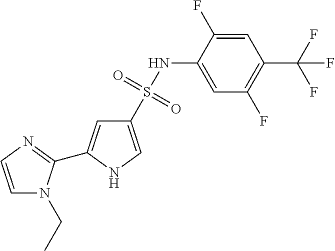 |
| Cpd 249 | 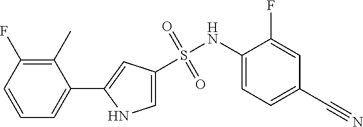 |
| Cpd 250 | 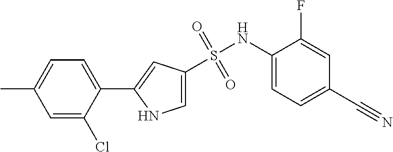 |
| Cpd 251 | 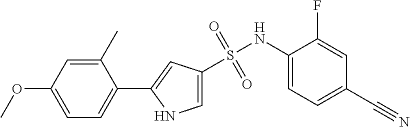 |
| Cpd 252 | 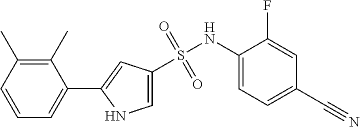 |
| Cpd 253 | 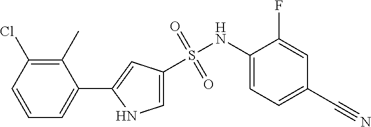 |
| Cpd 254 | 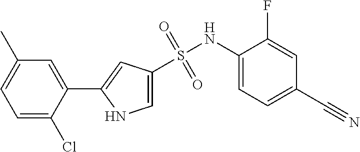 |
| Cpd 255 | 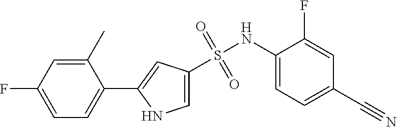 |
| Cpd 256 | 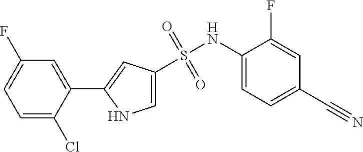 |
| Cpd 257 | 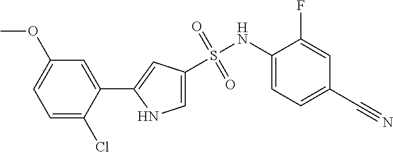 |
| Cpd 258 | 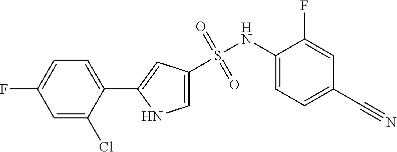 |
| Cpd 259 | 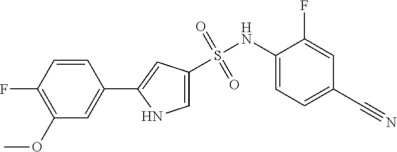 |
| Cpd 260 | 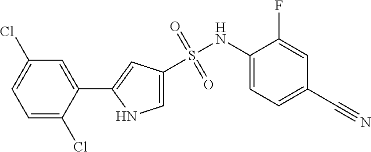 |
| Cpd 261 | 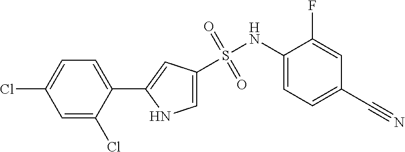 |
| Cpd 265 | 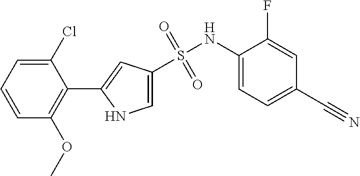 |
| Cpd 266 | 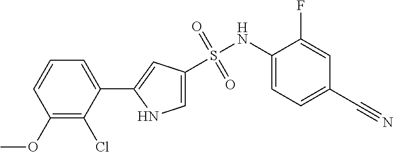 |
| Cpd 267 | 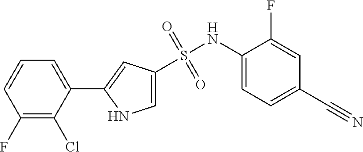 |
| Cpd 268 | 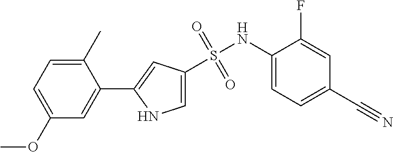 |
| Cpd 269 | 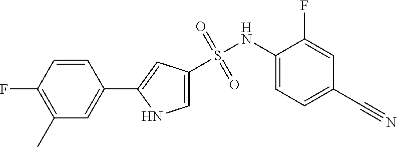 |
| Cpd 270 | 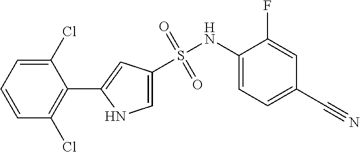 |
| Cpd 271 | 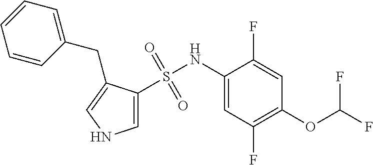 |
| Cpd 272 | 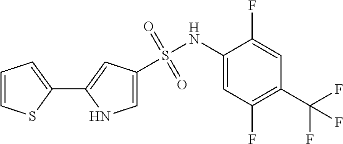 |
| Cpd 273 | 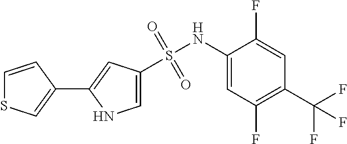 |
| Cpd 274 | 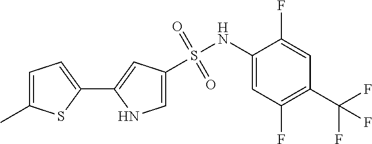 |
| Cpd 275 | 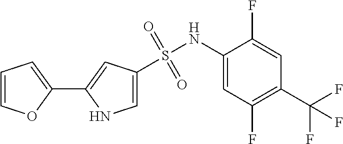 |
| Cpd 276 | 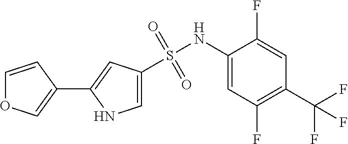 |
| Cpd 277 | 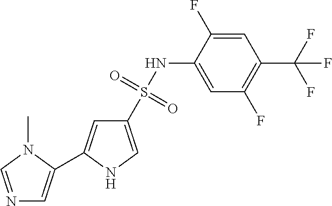 |
| Cpd 278 | 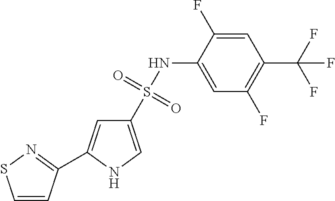 |
| Cpd 279 | 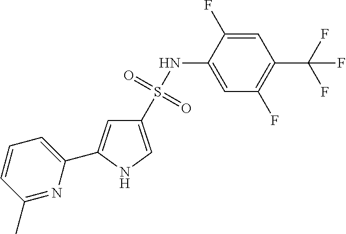 |
| Cpd 280 | 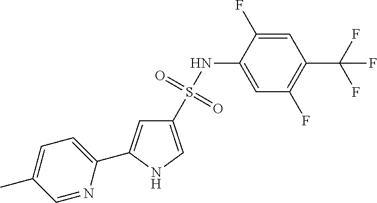 |
| Cpd 281 | 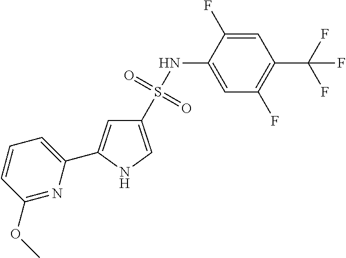 |
| Cpd 282 | 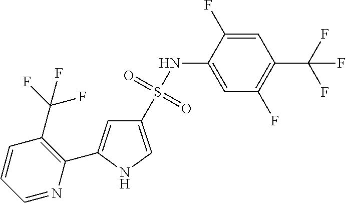 |
| Cpd 283 | 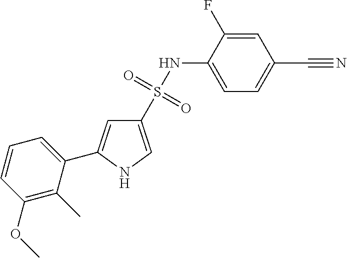 |
| Cpd 284 | 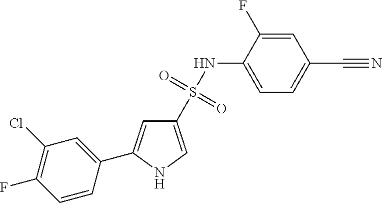 |
| Cpd 285 | 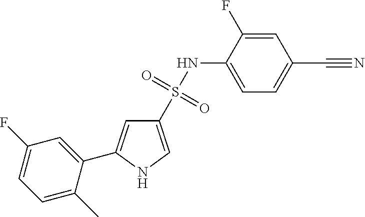 |
| Cpd 286 | 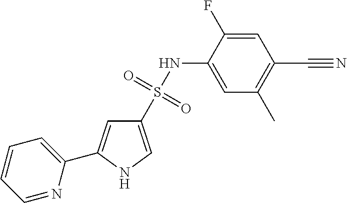 |
| Cpd 287 | 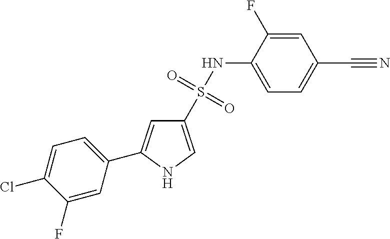 |
| Cpd 288 | 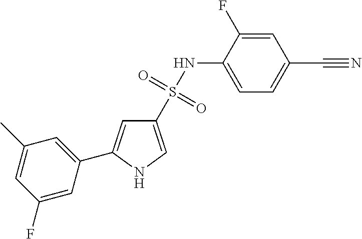 |
| Cpd 289 | 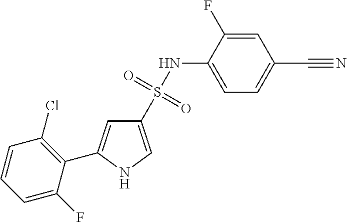 |
| Cpd 290 | 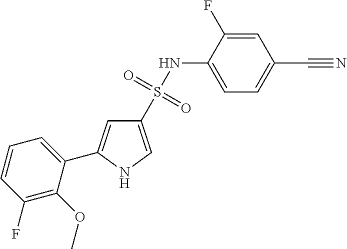 |
| Cpd 291 | 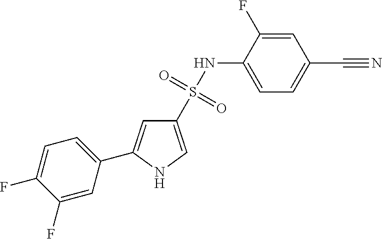 |
| Cpd 292 | 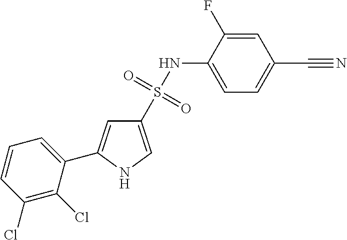 |
| Cpd 293 | 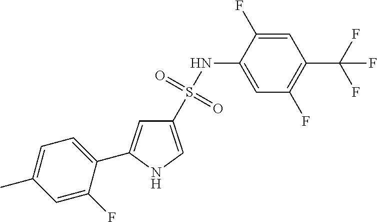 |
| Cpd 294 | 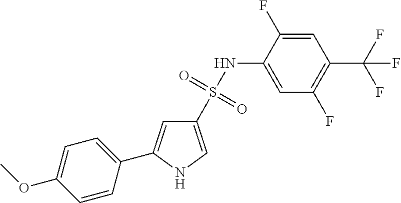 |
| Cpd 295 | 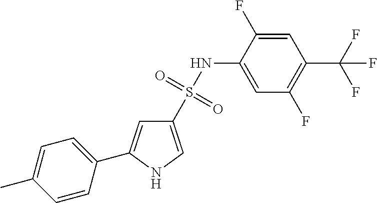 |
| Cpd 296 | 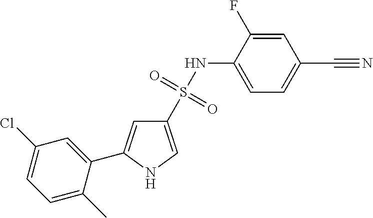 |
| Cpd 297 | 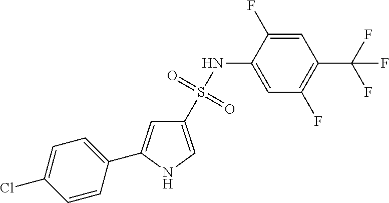 |
| Cpd 298 | 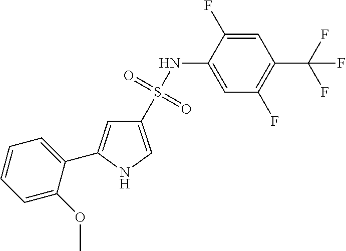 |
| Cpd 299 | 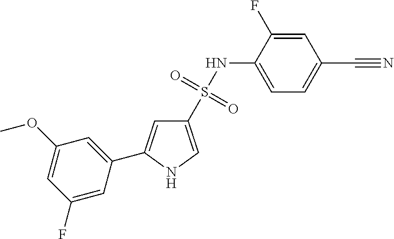 |
| Cpd 300 | 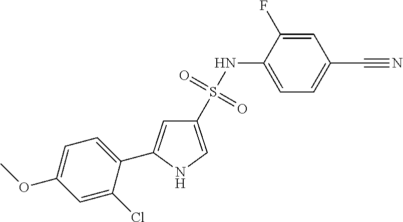 |
| Cpd 301 | 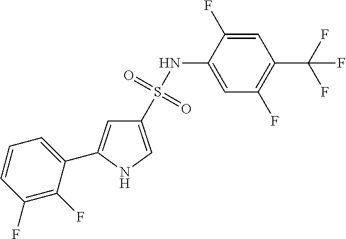 |
| Cpd 302 | 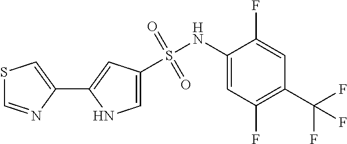 |
| Cpd 303 | 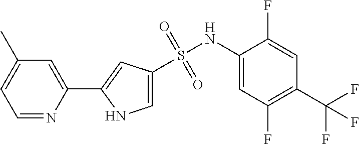 |
| Cpd 304 | 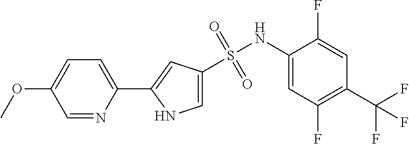 |
| Cpd 305 | 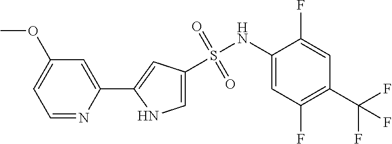 |
| Cpd 306 | 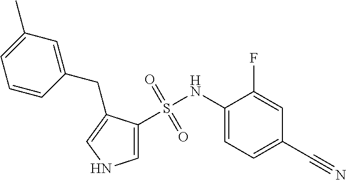 |
| Cpd 307 | 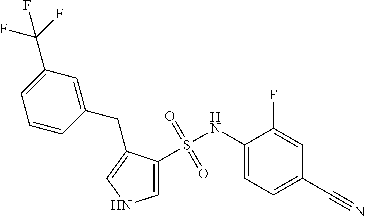 |
| Cpd 308 | 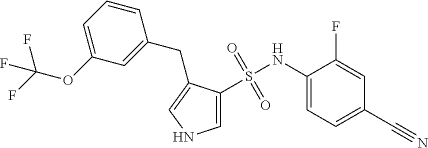 |
| Cpd 309 | 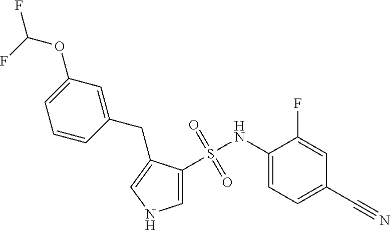 |
| Cpd 310 | 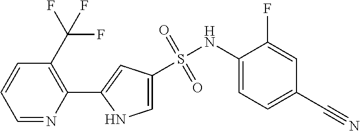 |
| Cpd 311 | 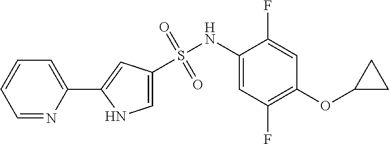 |
| Cpd 312 | 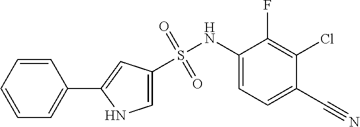 |
| Cpd 313 | 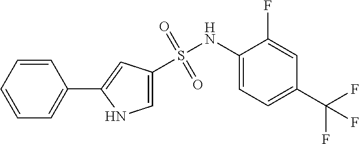 |
| Cpd 314 | 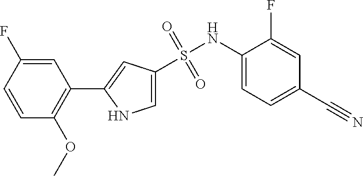 |
| Cpd 315 | 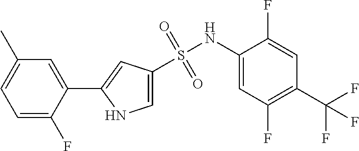 |
| Cpd 316 | 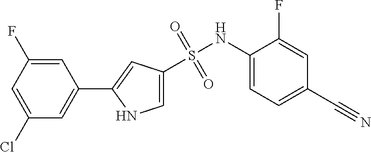 |
| Cpd 317 | 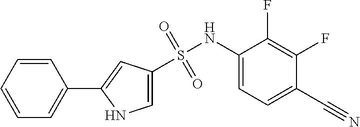 |
| Cpd 318 | 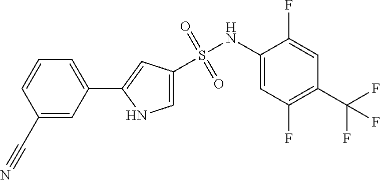 |
| Cpd 319 | 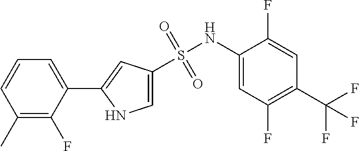 |
| Cpd 320 | 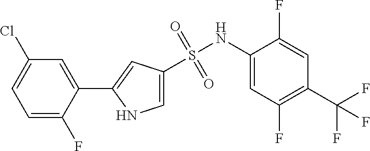 |
| Cpd 321 | 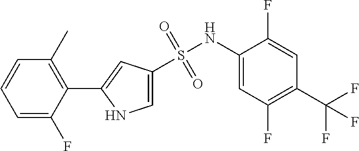 |
| Cpd 322 | 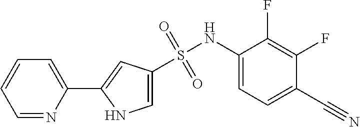 |
| Cpd 323 | 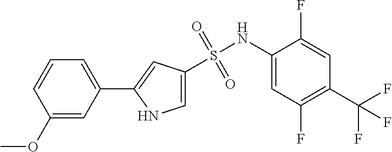 |
| Cpd 324 | 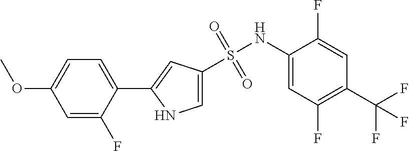 |
| Cpd 325 | 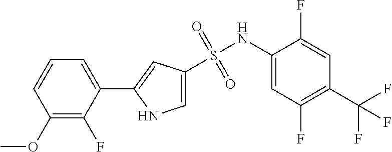 |
| Cpd 326 | 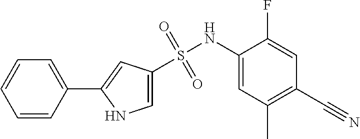 |
| Cpd 327 | 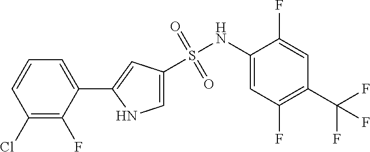 |
| Cpd 328 | 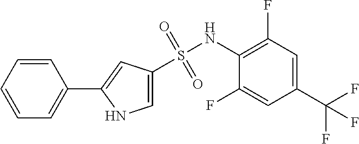 |
| Cpd 329 | 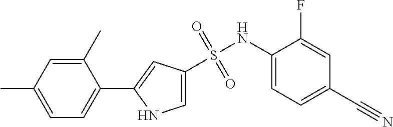 |
| Cpd 330 | 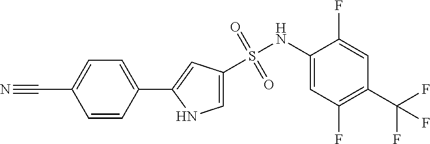 |
| Cpd 331 | 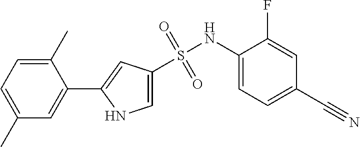 |
| Cpd 332 | 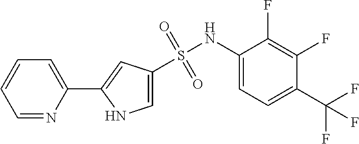 |
| Cpd 333 | 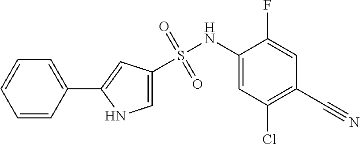 |
| Cpd 334 | 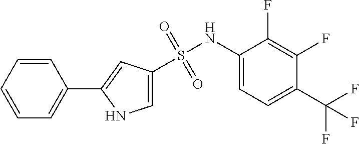 |
| Cpd 335 | 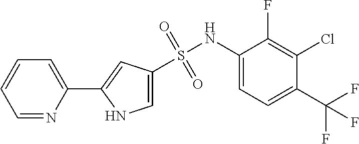 |
| Cpd 336 | 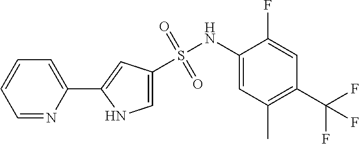 |
| Cpd 337 | 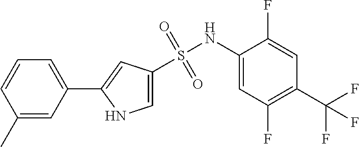 |
| Cpd 338 | 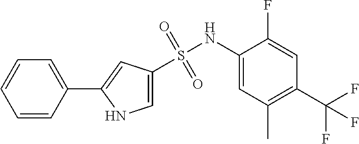 |
| Cpd 339 | 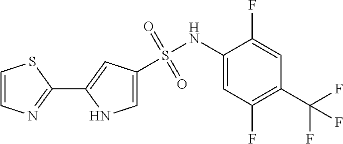 |
| Cpd 340 | 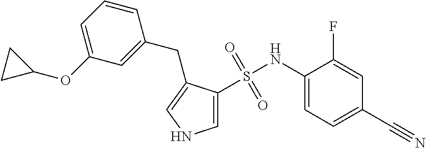 |
| Cpd 341 | 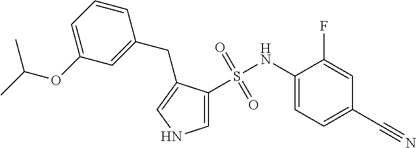 |
| Cpd 342 | 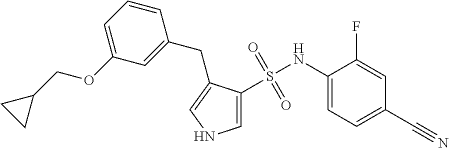 |
| Cpd 343 | 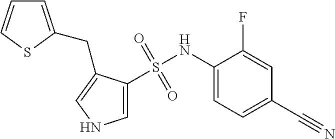 |
| Cpd 344 | 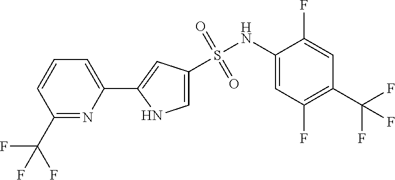 |
| Cpd 345 | 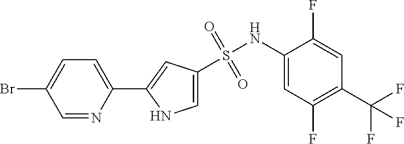 |
| Cpd 346 | 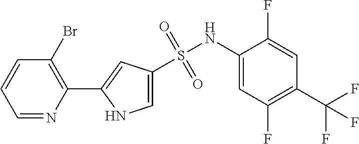 |
| Cpd 347 | 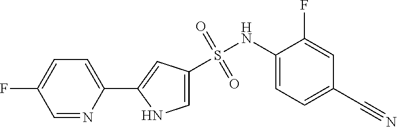 |
| Cpd 348 | 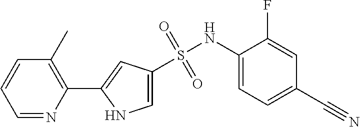 |
| Cpd 349 | 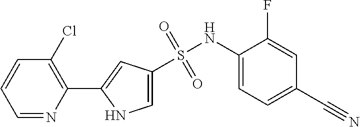 |
| Cpd 350 | 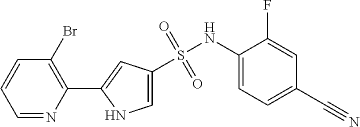 |
| Cpd 351 | 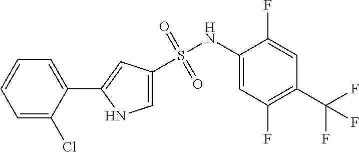 |
| Cpd 352 | 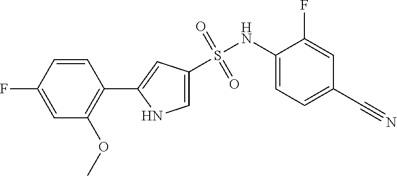 |
| Cpd 353 | 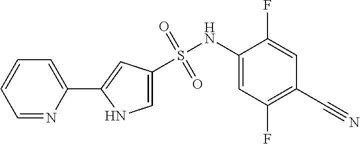 |
| Cpd 354 | 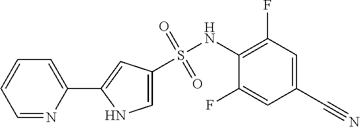 |
| Cpd 355 | 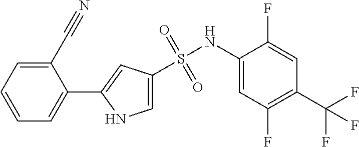 |
| Cpd 356 | 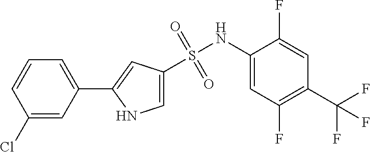 |
| Cpd 357 | 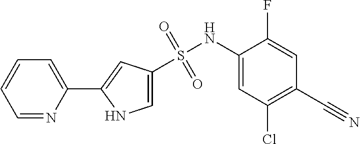 |
| Cpd 358 | 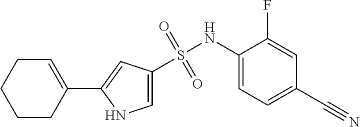 |
| Cpd 359 | 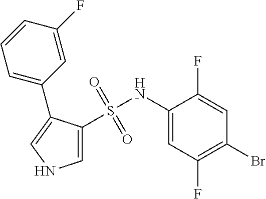 |
| Cpd 360 | 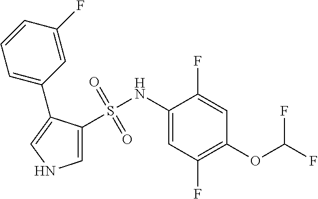 |
| Cpd 361 | 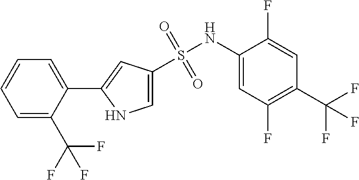 |
| Cpd 362 | 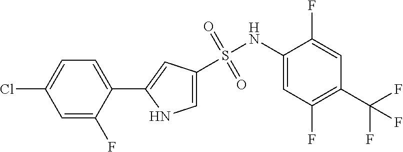 |
| Cpd 363 | 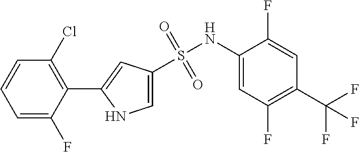 |
| Cpd 364 | 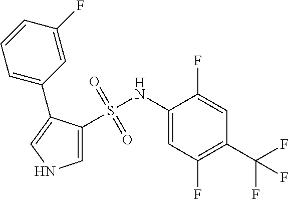 |
| Cpd 365 | 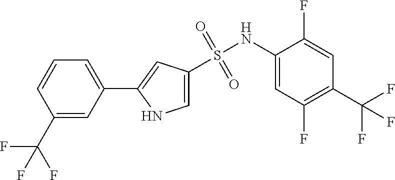 |
| Cpd 366 | 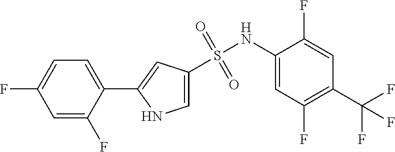 |
| Cpd 367 | 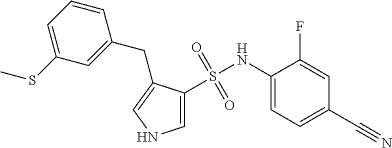 |
| Cpd 368 | 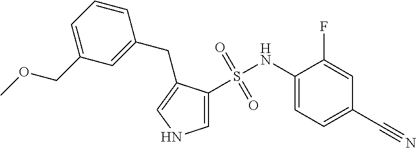 |
| Cpd 369 | 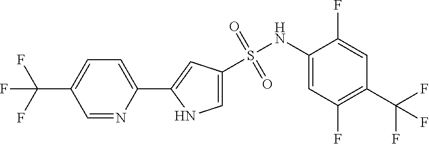 |
| Cpd 370 | 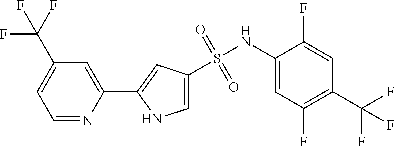 |
| Cpd 371 | 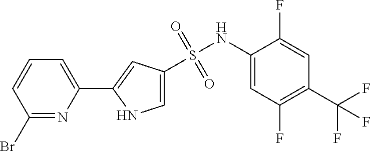 |
| Cpd 372 | 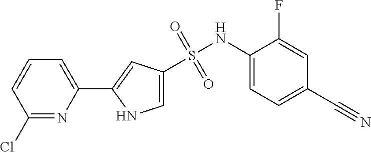 |
| Cpd 373 | 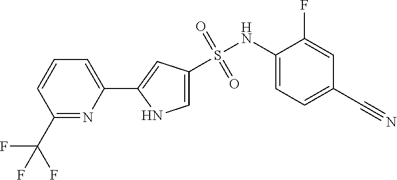 |
| Cpd 374 | 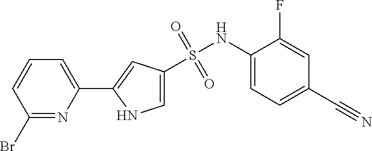 |
| Cpd 375 | 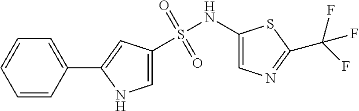 |
| Cpd 378 | 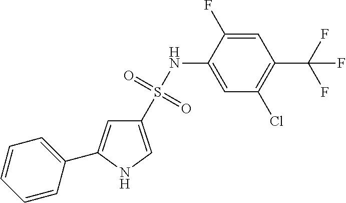 |
| Cpd 379 | 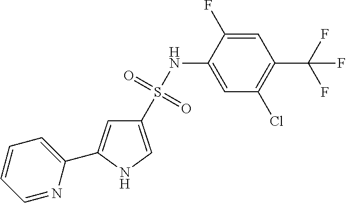 |
| Cpd 380 | 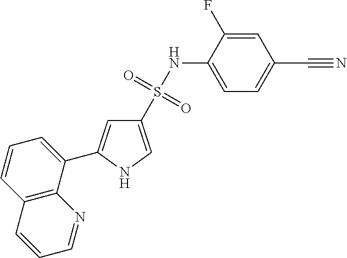 |
| Cpd 381 | 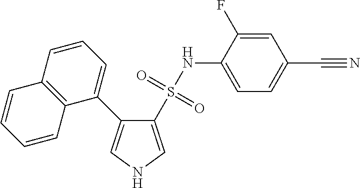 |
| Cpd 382 | 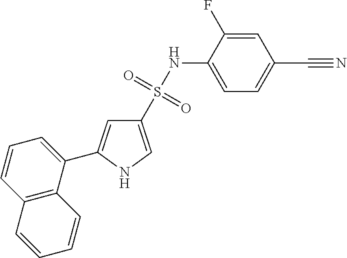 |
| Cpd 383 | 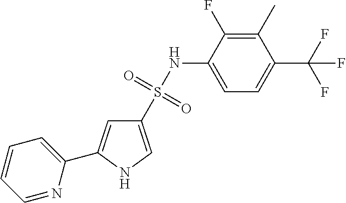 |
| Cpd 384 | 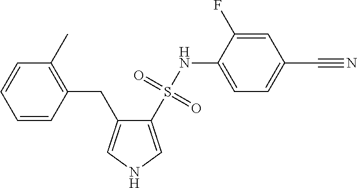 |
| Cpd 385 | 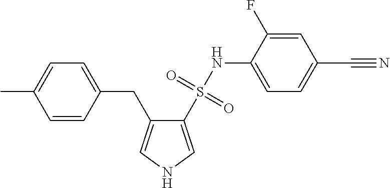 |
| Cpd 386 | 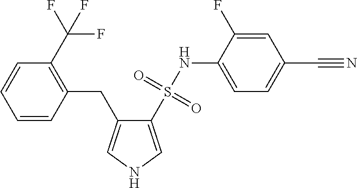 |
| Cpd 387 | 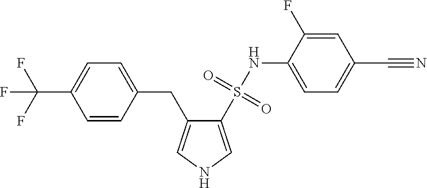 |
| Cpd 388 | 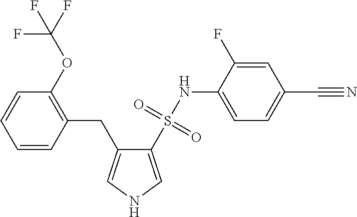 |
| Cpd 389 | 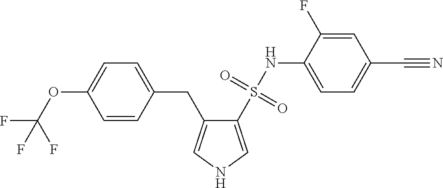 |
| Cpd 390 | 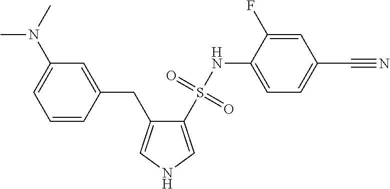 |
| Cpd 391 | 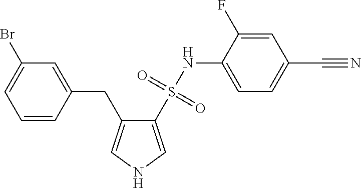 |
| Cpd 392 | 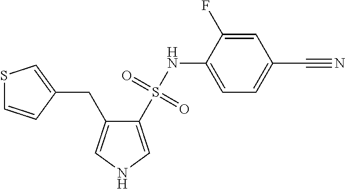 |
| Cpd 393 | 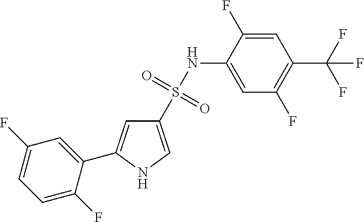 |
| Cpd 394 | 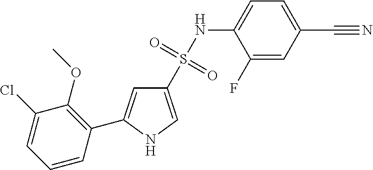 |
| Cpd 395 | 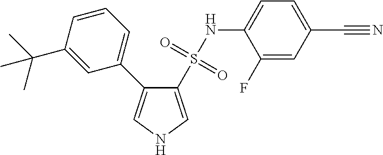 |
| Cpd 396 | 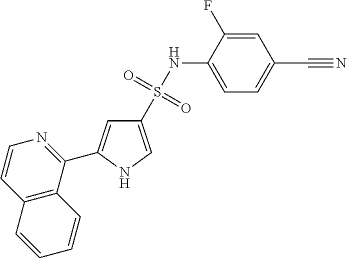 |
| Cpd 397 | 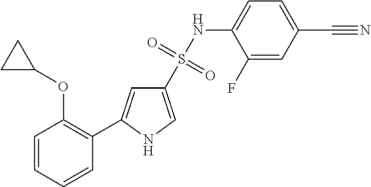 |
| Cpd 398 | 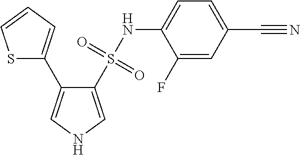 |
| Cpd 399 | 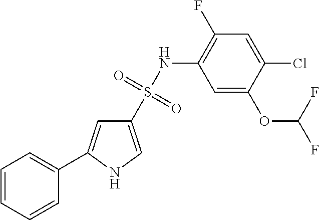 |
| Cpd 400 | 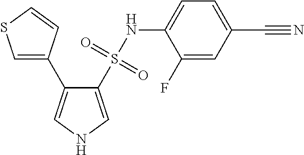 |
| Cpd 401 | 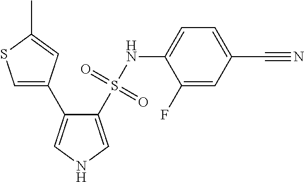 |
| Cpd 402 | 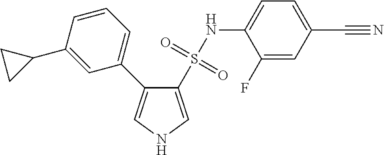 |
| Cpd 403 | 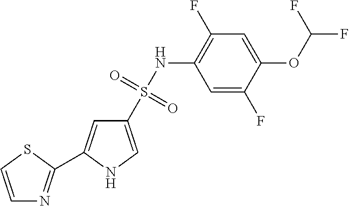 |
| Cpd 404 | 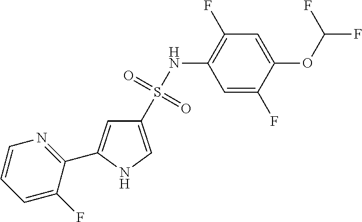 |
| Cpd 405 | 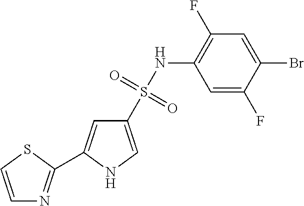 |
| Cpd 406 | 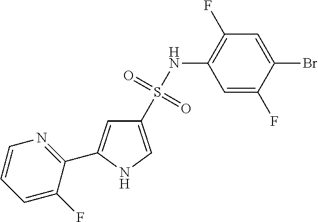 |
| Cpd 407 | 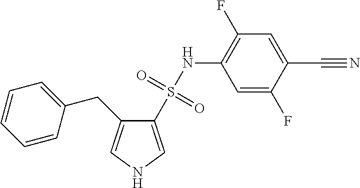 |
| Cpd 408 | 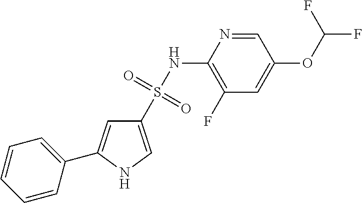 |
| Cpd 409 | 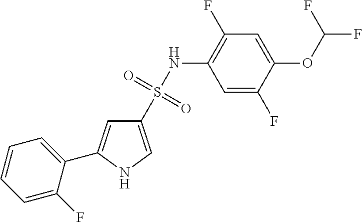 |
| Cpd 410 | 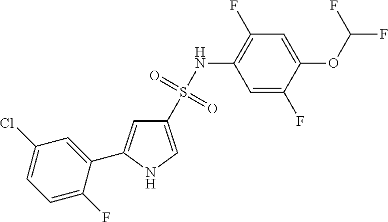 |
| Cpd 411 | 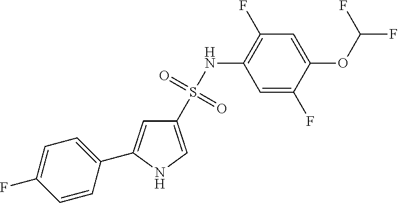 |
| Cpd 412 | 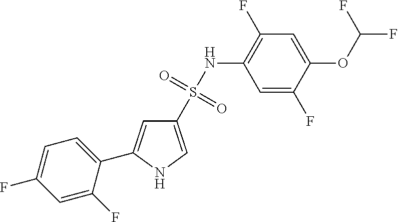 |
| Cpd 413 | 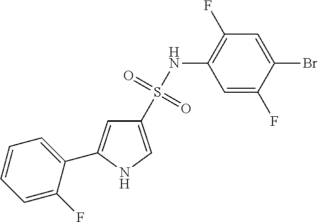 |
| Cpd 414 | 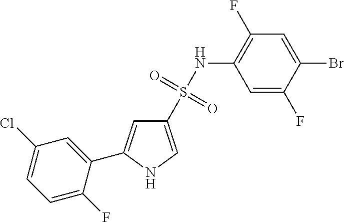 |
| Cpd 415 | 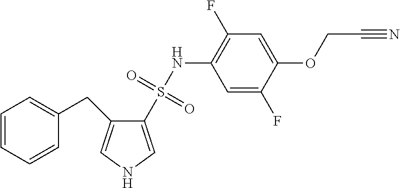 |
| Cpd 416 | 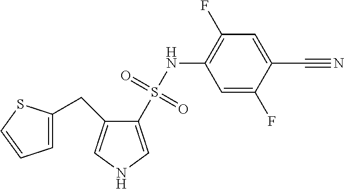 |
| Cpd 417 | 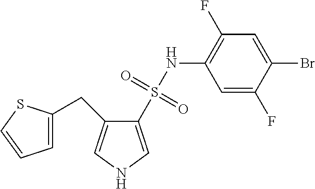 |
| Cpd 418 | 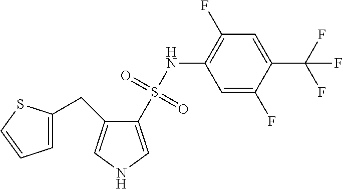 |
| Cpd 419 | 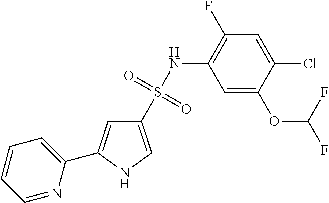 |
| Cpd 420 | 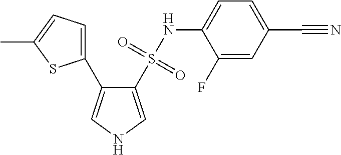 |
| Cpd 421 | 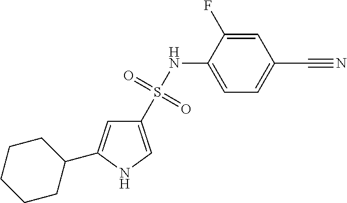 |
| Cpd 422 | 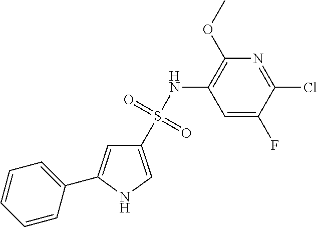 |
| Cpd 423 | 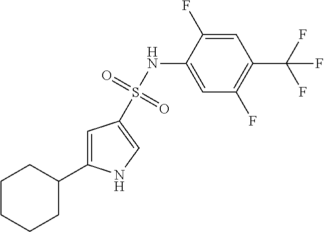 |
| Cpd 424 | 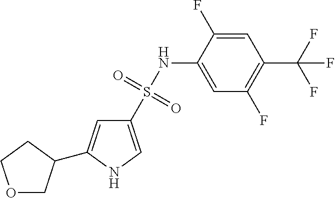 |
| Cpd 425 | 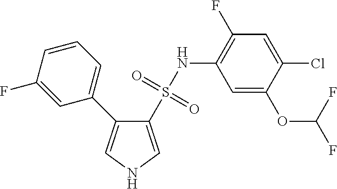 |
| Cpd 426 | 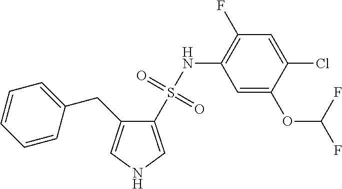 |
| Cpd 427 | 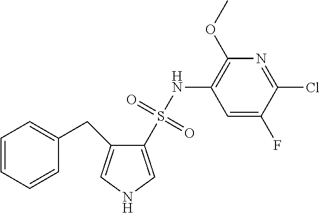 |
| Cpd 428 | 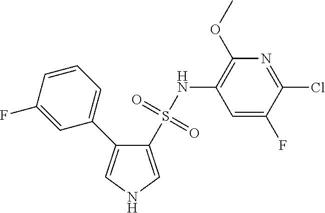 |
| Cpd 429 | 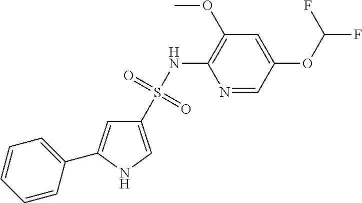 |
| Cpd 430 | 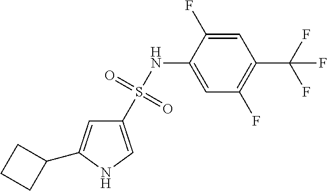 |
| Cpd 431 | 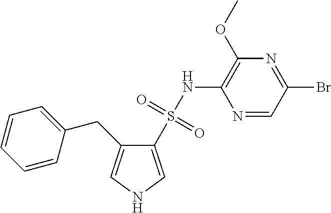 |
| Cpd 432 | 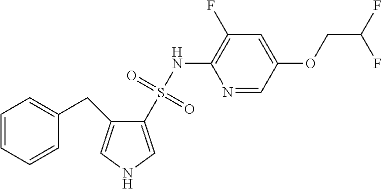 |
| Cpd 433 | 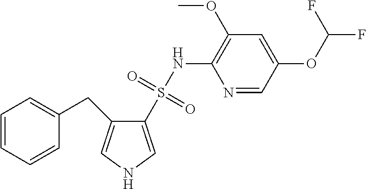 |
| Cpd 434 | 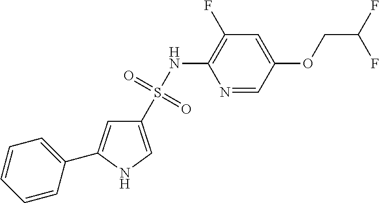 |
| Cpd 435 | 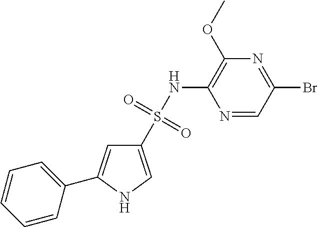 |
| Cpd 436 | 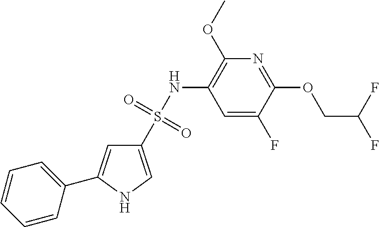 |
| Cpd 437 | 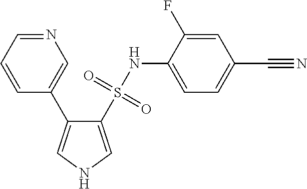 |
| Cpd 438 | 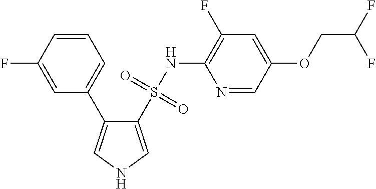 |
| Cpd 439 | 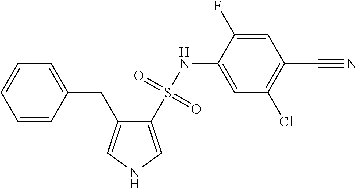 |
| Cpd 440 | 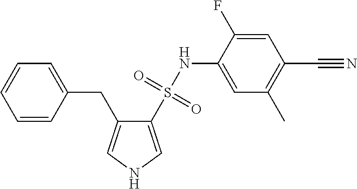 |
| Cpd 441 | 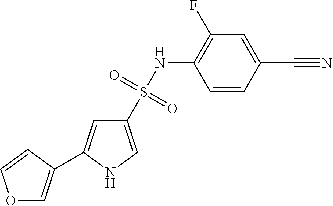 |
| Cpd 442 | 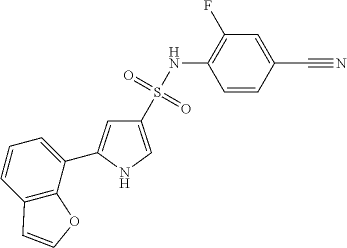 |
| Cpd 443 | 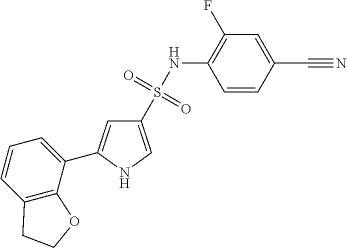 |
| Cpd 444 | 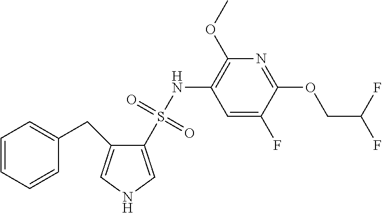 |
| Cpd 445 | 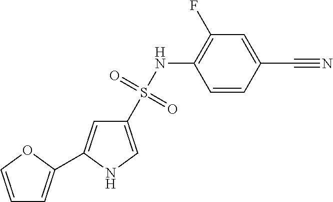 |
| Cpd 446 | 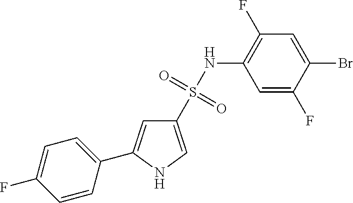 |
| Cpd 447 | 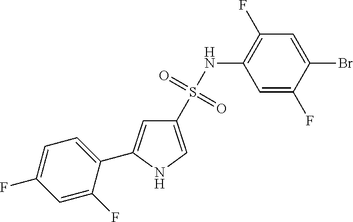 |
| Cpd 448 | 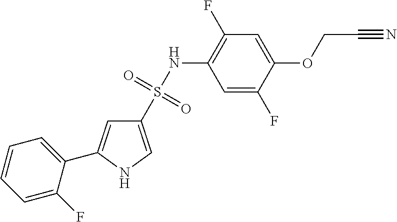 |
| Cpd 449 | 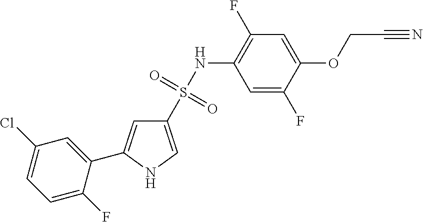 |
| Cpd 450 | 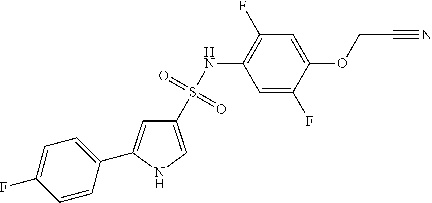 |
| Cpd 451 | 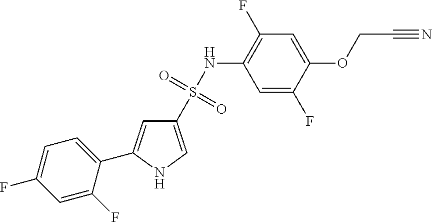 |
| Cpd 452 | 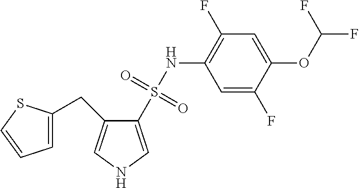 |
| Cpd 453 | 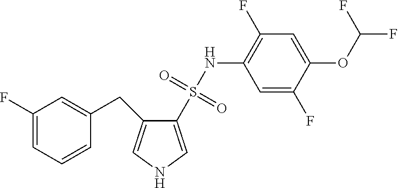 |
| Cpd 454 | 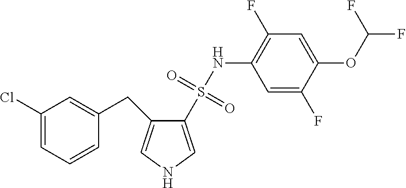 |
| Cpd 455 | 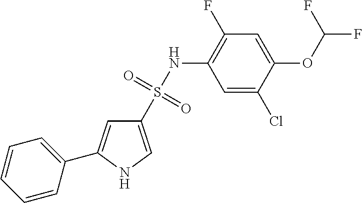 |
| Cpd 456 | 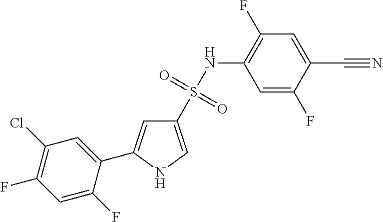 |
| Cpd 457 | 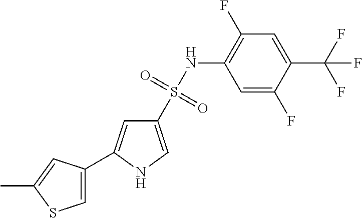 |
| Cpd 458 | 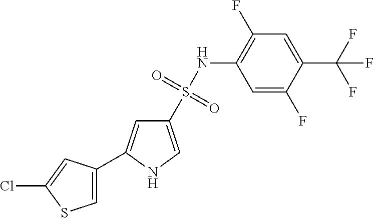 |
| Cpd 459 | 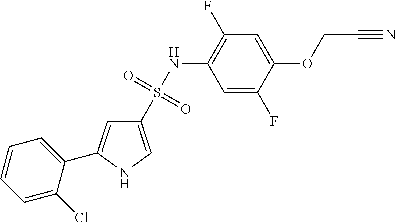 |
| Cpd 460 | 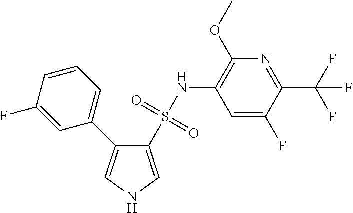 |
| Cpd 461 | 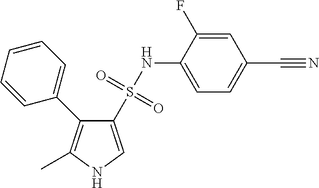 |
| Cpd 462 | 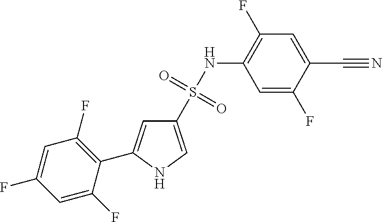 |
| Cpd 463 | 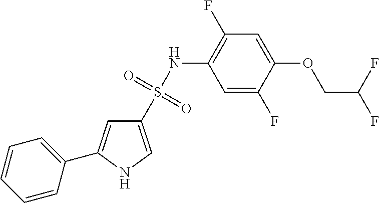 |
| Cpd 464 | 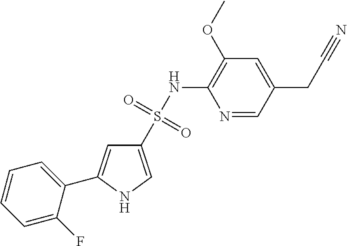 |
| Cpd 465 | 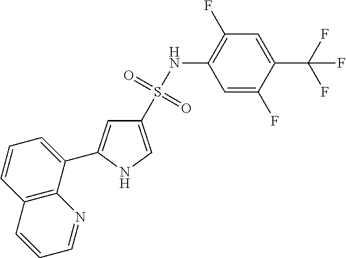 |
| Cpd 466 | 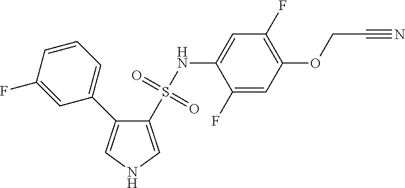 |
| Cpd 467 | 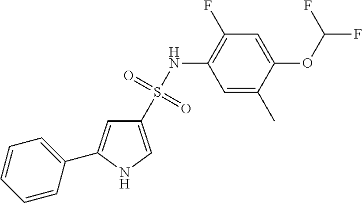 |
| Cpd 468 | 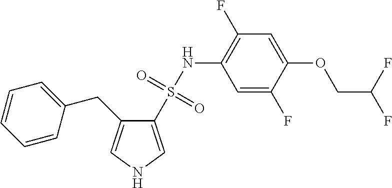 |
| Cpd 469 | 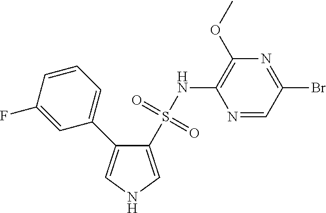 |
| Cpd 470 | 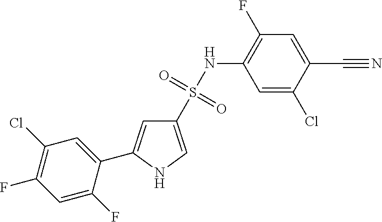 |
| Cpd 471 | 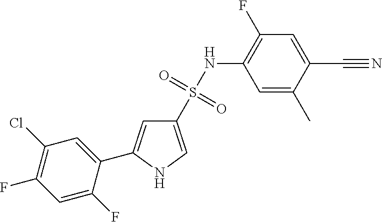 |
| Cpd 472 | 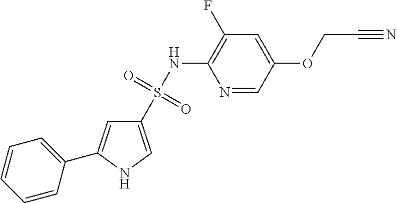 |
| Cpd 473 | 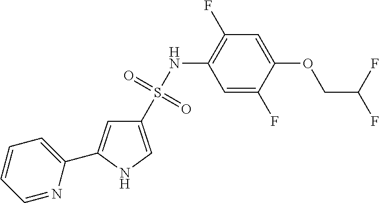 |
| Cpd 474 | 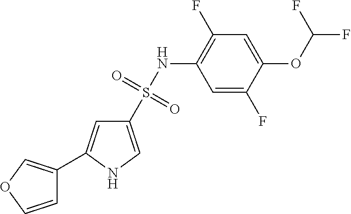 |
| Cpd 475 | 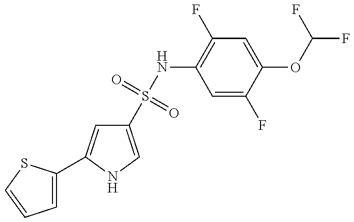 |
| Cpd 476 | 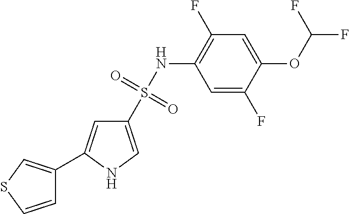 |
| Cpd 477 | 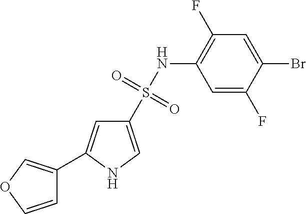 |
| Cpd 478 | 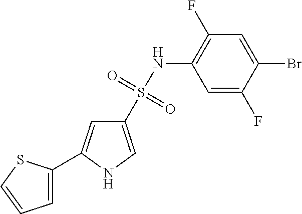 |
| Cpd 479 | 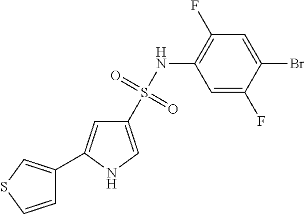 |
| Cpd 480 | 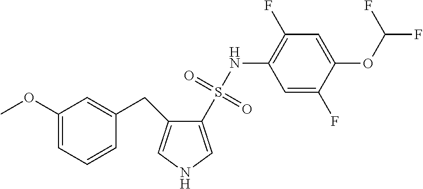 |
| Cpd 481 | 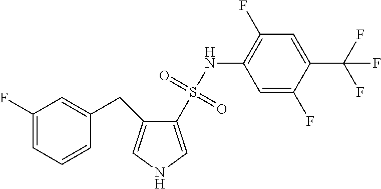 |
| Cpd 482 | 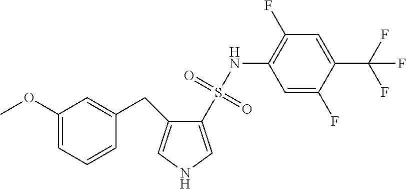 |
| Cpd 483 | 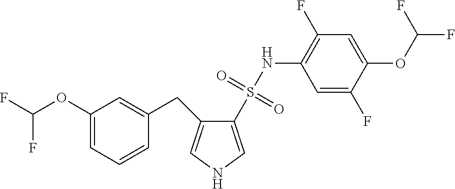 |
| Cpd 484 | 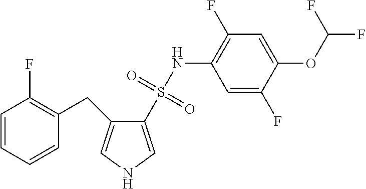 |
| Cpd 485 | 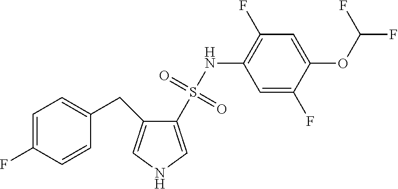 |
| Cpd 486 | 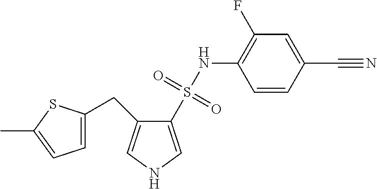 |
| Cpd 487 | 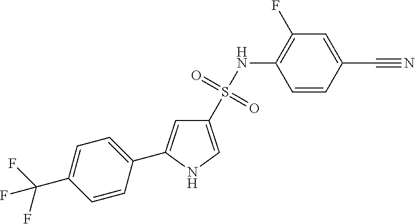 |
| Cpd 488 | 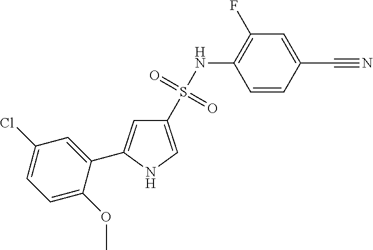 |
| Cpd 489 | 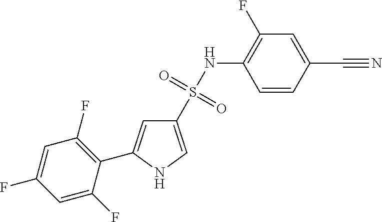 |
| Cpd 490 | 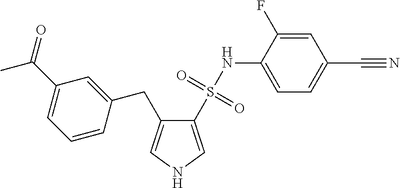 |
| Cpd 491 | 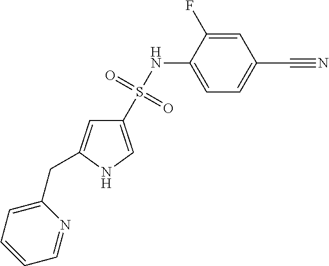 |
| Cpd 492 | 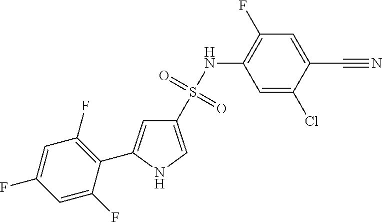 |
| Cpd 493 | 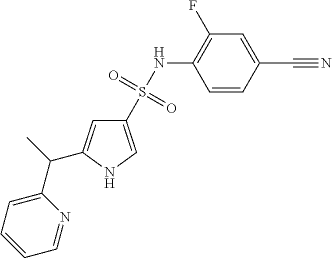 |
| Cpd 494 | 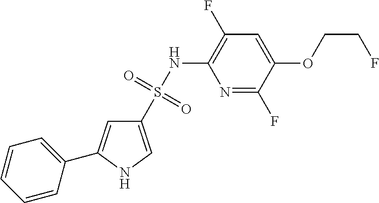 |
| Cpd 495 | 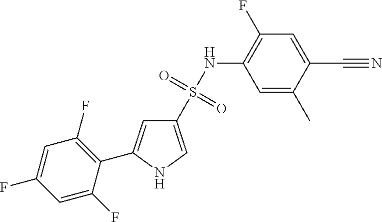 |
| Cpd 496 | 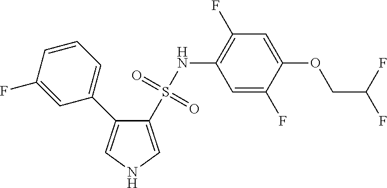 |
| Cpd 497 | 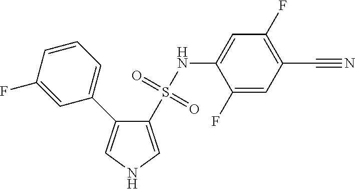 |
| Cpd 498 | 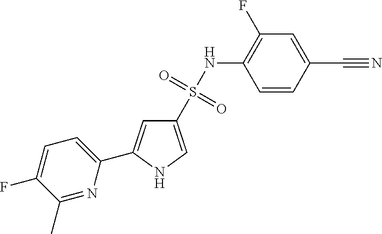 |
| Cpd 499 | 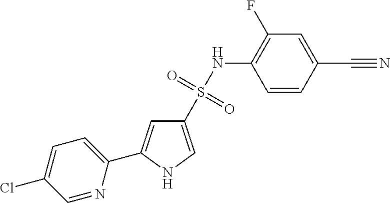 |
| Cpd 500 | 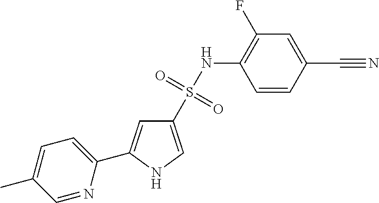 |
| Cpd 501 | 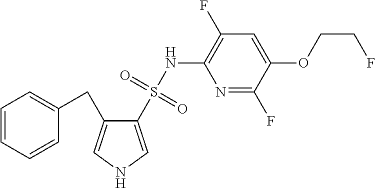 |
| Cpd 502 | 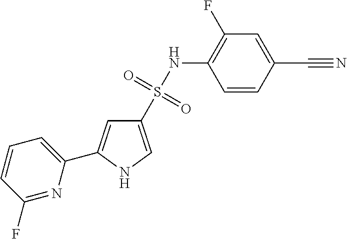 |
| Cpd 503 | 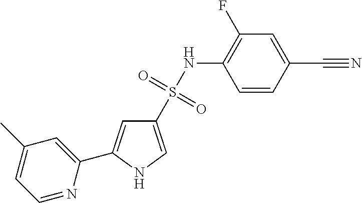 |
| Cpd 504 | 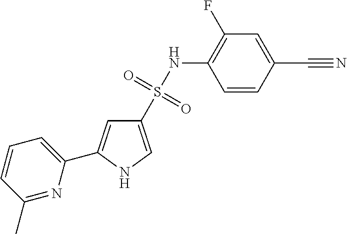 |
| Cpd 505 | 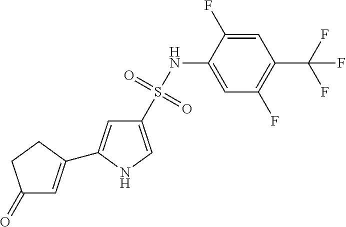 |
| Cpd 506 | 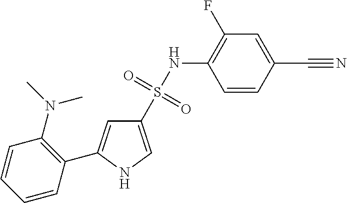 |
| Cpd 507 | 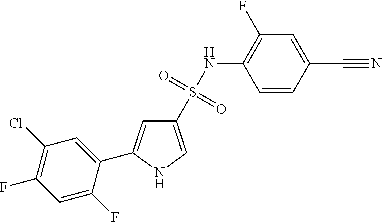 |
| Cpd 508 | 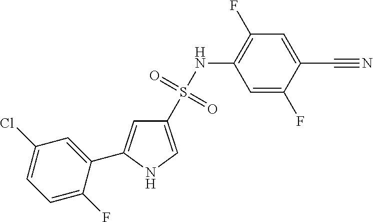 |
| Cpd 509 | 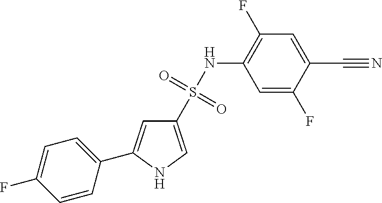 |
| Cpd 510 | 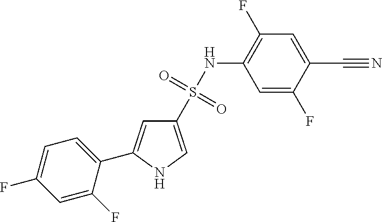 |
| Cpd 511 | 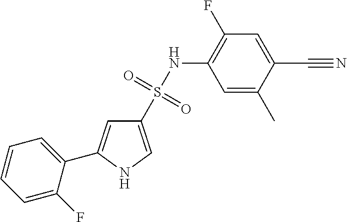 |
| Cpd 512 | 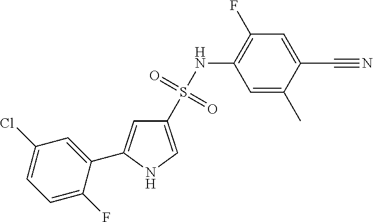 |
| Cpd 513 | 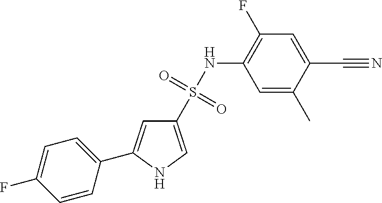 |
| Cpd 514 | 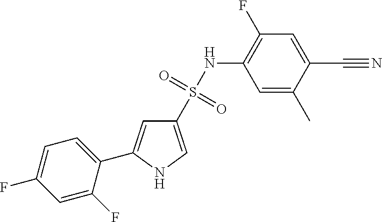 |
| Cpd 515 | 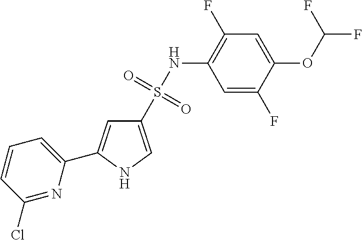 |
| Cpd 516 | 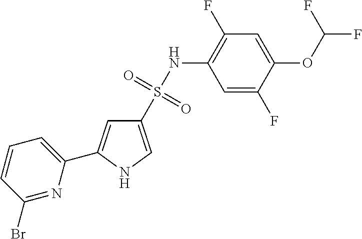 |
| Cpd 517 | 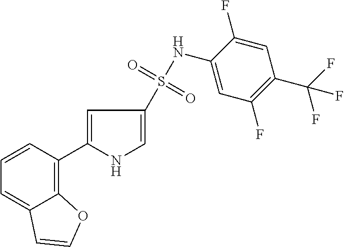 |
| Cpd 518 | 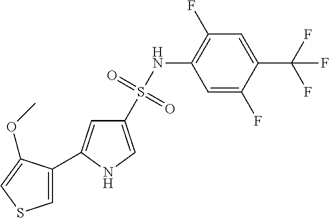 |
| Cpd 519 | 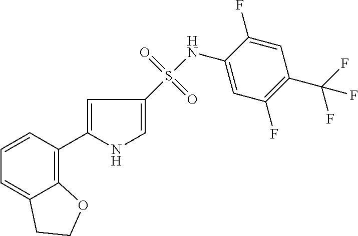 |
| Cpd 520 | 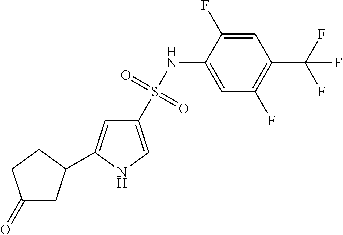 |
| Cpd 521 | 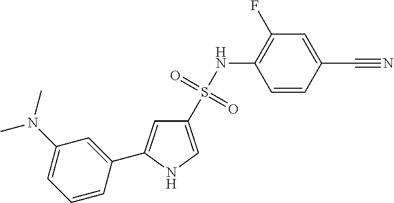 |
| Cpd 522 | 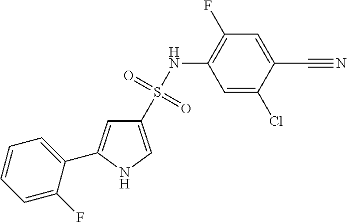 |
| Cpd 523 | 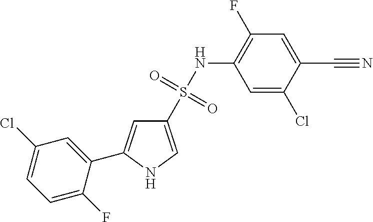 |
| Cpd 524 | 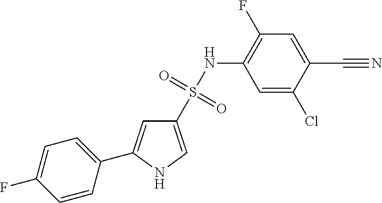 |
| Cpd 525 | 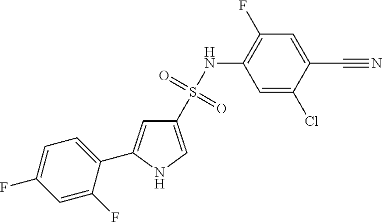 |
| Cpd 526 | 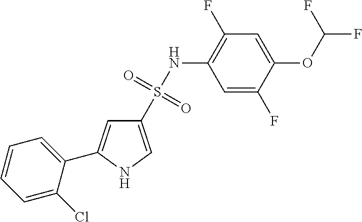 |
| Cpd 527 | 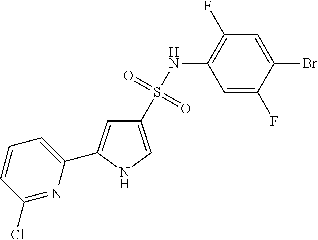 |
| Cpd 528 | 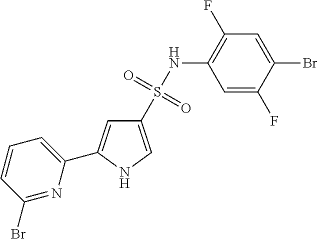 |
| Cpd 529 | 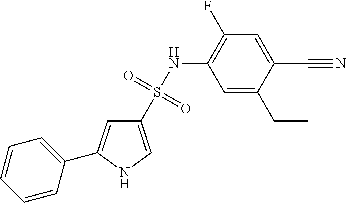 |
| Cpd 530 | 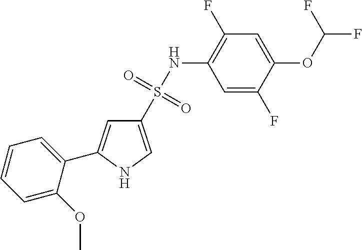 |
| Cpd 531 | 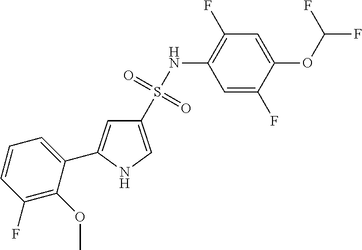 |
| Cpd 532 | 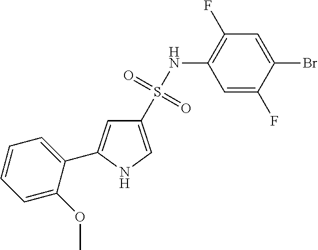 |
| Cpd 533 | 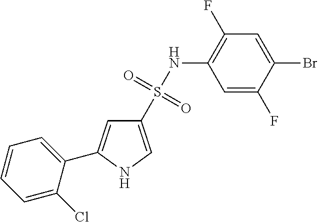 |
| Cpd 534 | 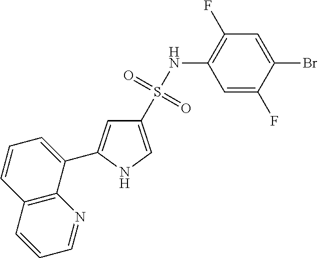 |
| Cpd 535 | 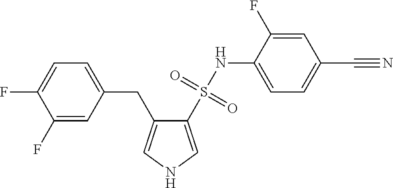 |
| Cpd 536 | 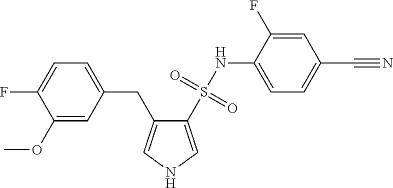 |
| Cpd 537 | 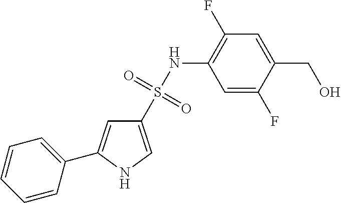 |
| Cpd 538 | 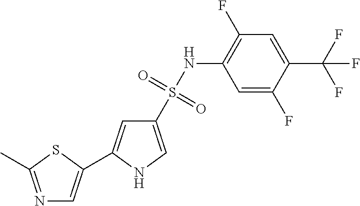 |
| Cpd 539 | 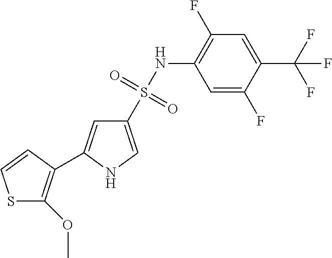 |
| Cpd 540 | 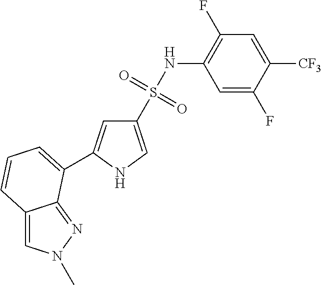 |
| Cpd 541 | 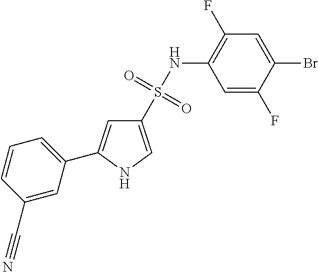 |
| Cpd 542 | 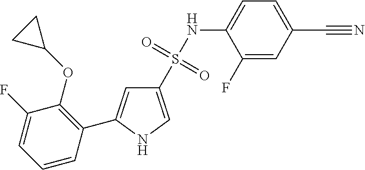 |
| Cpd 543 | 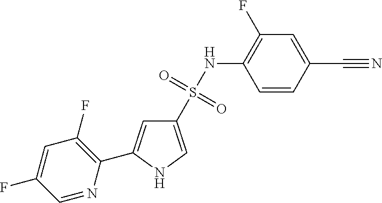 |
| Cpd 544 | 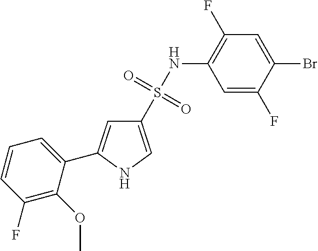 |
| Cpd 545 | 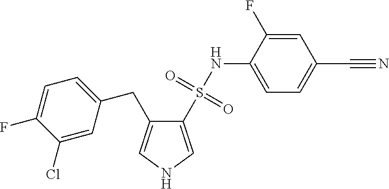 |
| Cpd 546 | 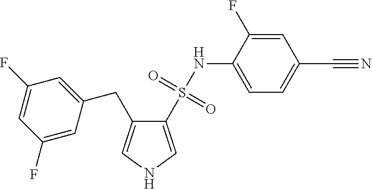 |
| Cpd 547 | 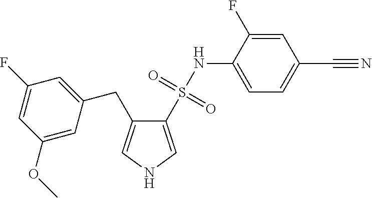 |
| Cpd 548 | 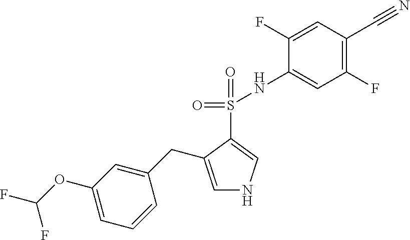 |
| Cpd 549 | 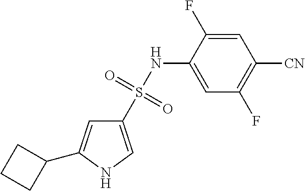 |
| Cpd 550 | 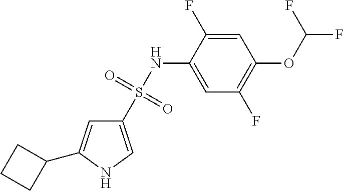 |
| Cpd 551 | 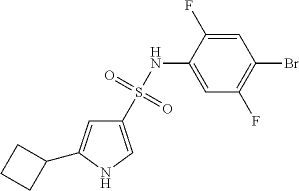 |
| Cpd 552 | 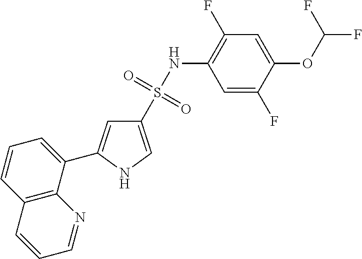 |
| Cpd 553 | 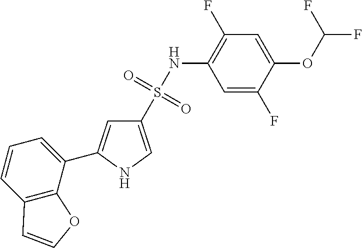 |
| Cpd 554 | 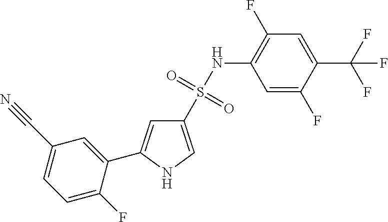 |
| Cpd 555 | 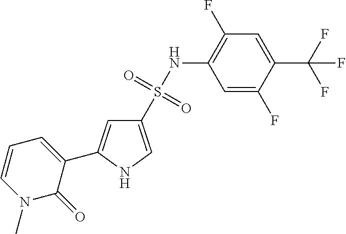 |
| Cpd 556 | 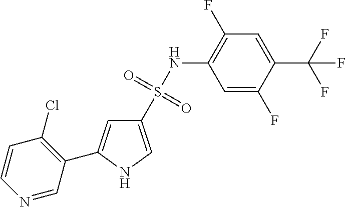 |
| Cpd 557 | 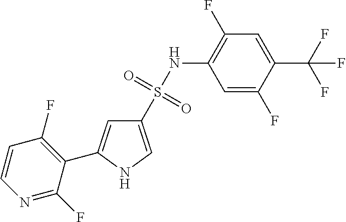 |
| Cpd 558 | 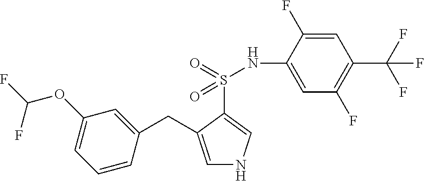 |
| Cpd 559 | 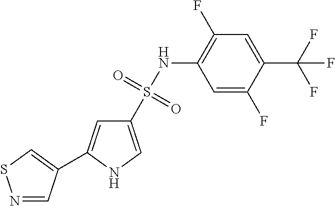 |
| Cpd 560 | 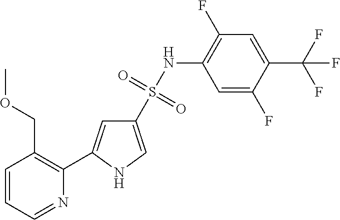 |
| Cpd 561 | 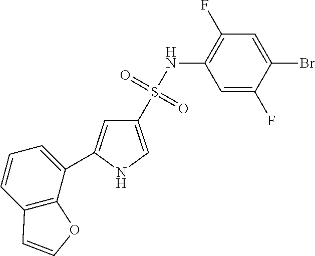 |
| Cpd 562 | 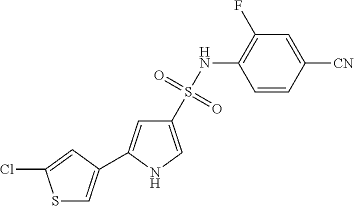 |
| Cpd 563 | 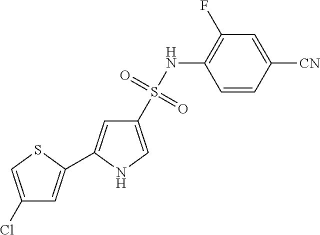 |
| Cpd 564 | 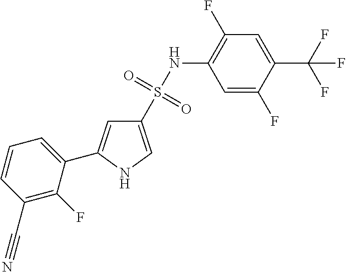 |
| Cpd 565 | 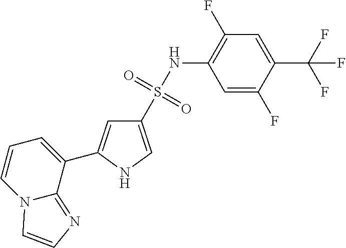 |
| Cpd 566 | 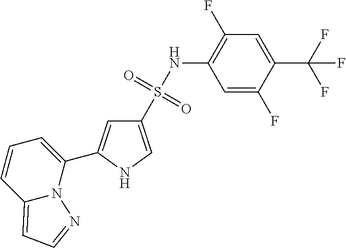 |
| Cpd 567 | 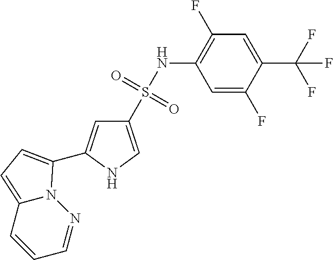 |
| Cpd 568 | 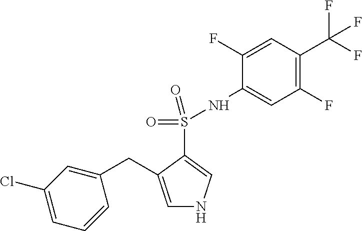 |
| Cpd 569 | 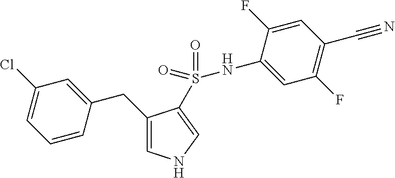 |
| Cpd 570 | 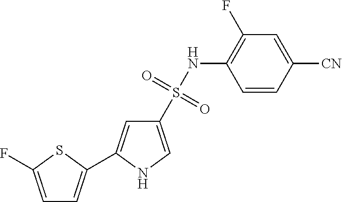 |
| Cpd 571 | 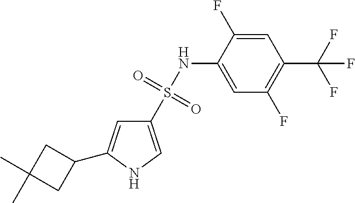 |
| Cpd 572 | 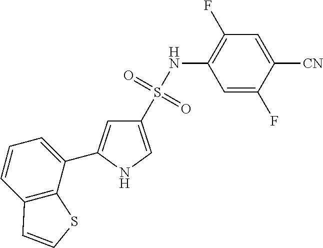 |
| Cpd 573 | 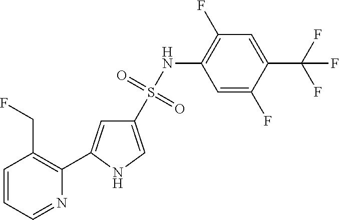 |
| Cpd 574 | 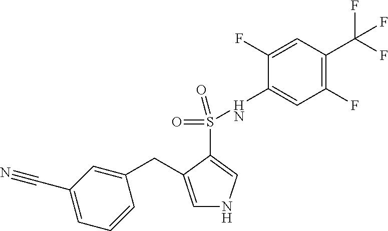 |
| Cpd 575 | 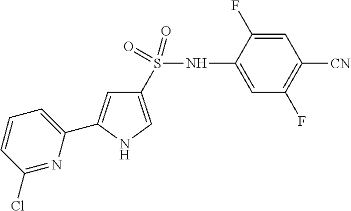 |
| Cpd 576 | 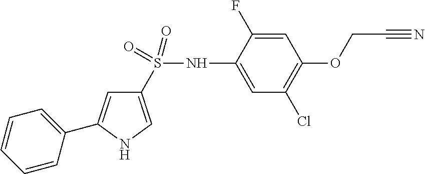 |
| Cpd 577 | 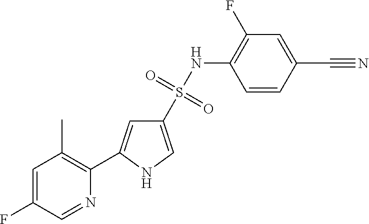 |
| Cpd 578 | 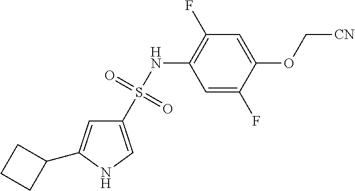 |
| Cpd 579 | 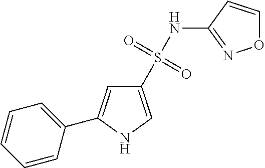 |
| Cpd 580 | 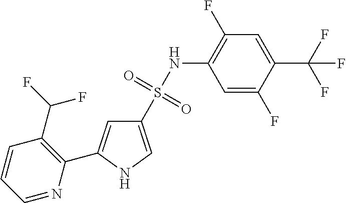 |
| Cpd 581 | 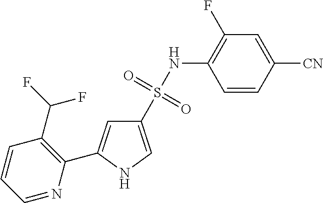 |
| Cpd 582 | 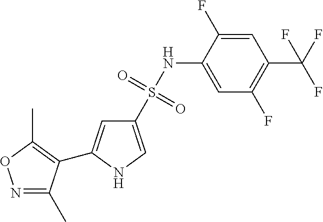 |
| Cpd 583 | 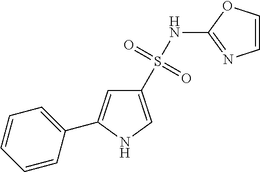 |
| Cpd 584 | 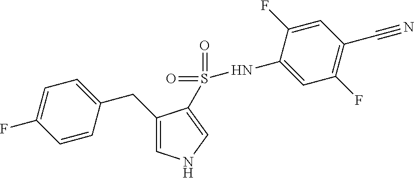 |
| Cpd 585 | 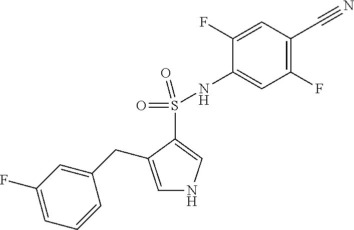 |
| Cpd 586 | 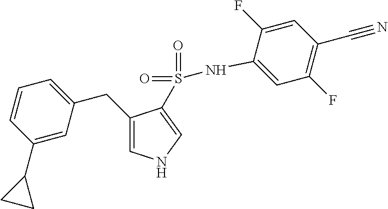 |
| Cpd 587 | 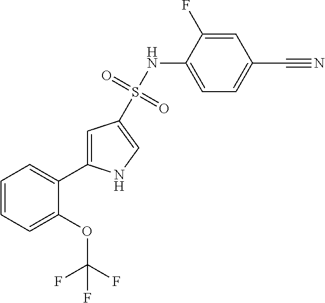 |
| Cpd 588 | 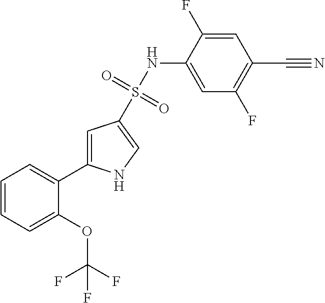 |
| Cpd 589 | 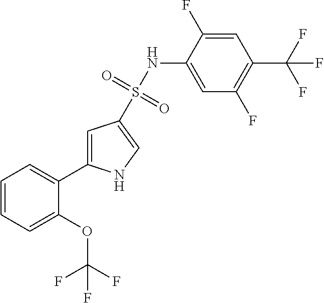 |
| Cpd 590 | 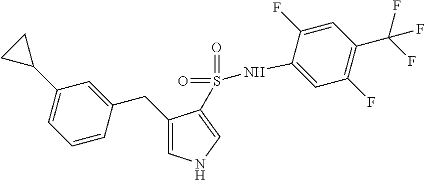 |
| Cpd 591 | 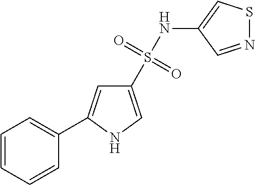 |
| Cpd 592 | 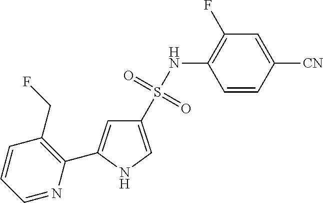 |
| Cpd 593 | 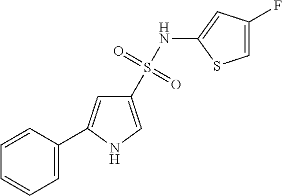 |
| Cpd 594 | 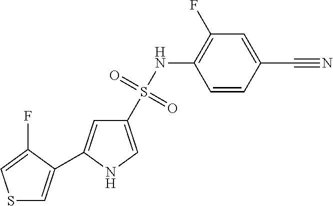 |
| Cpd 595 | 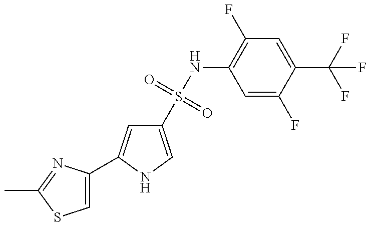 |
| Cpd 596 | 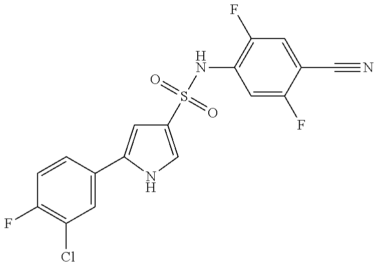 |
| Cpd 597 | 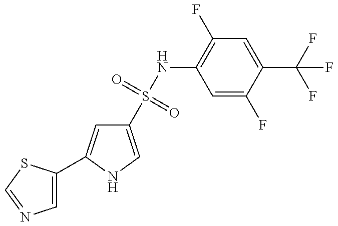 |
| Cpd 598 | 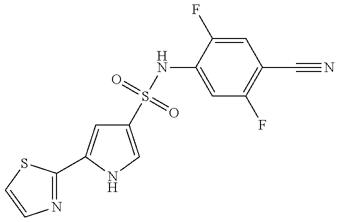 |
| Cpd 599 | 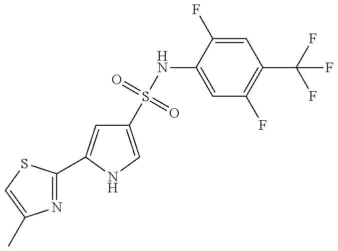 |
| Cpd 600 | 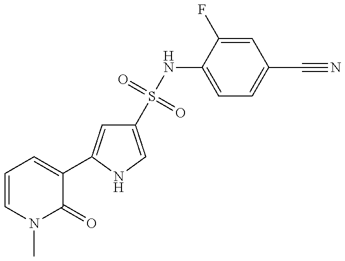 |
| Cpd 601 | 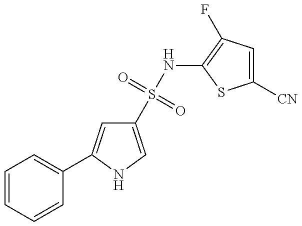 |
| Cpd 602 | 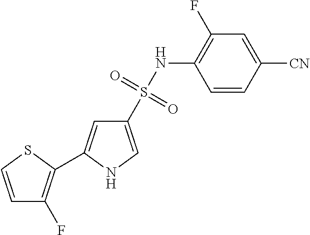 |
| Cpd 603 | 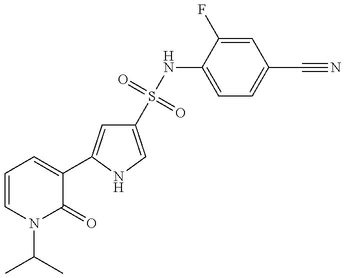 |
| Cpd 604 | 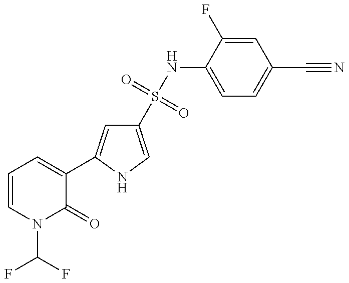 |
| Cpd 605 | 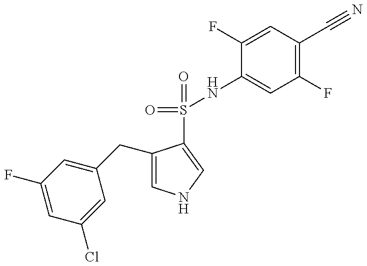 |
| Cpd 606 | 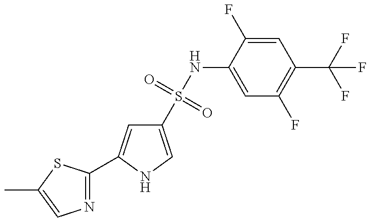 |
| Cpd 607 | 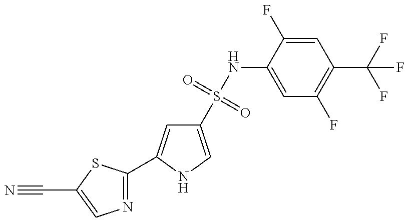 |
| Cpd 608 | 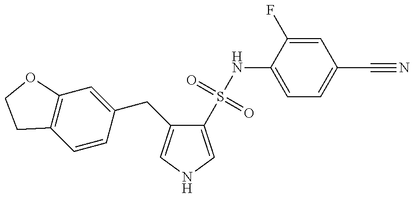 |
| Cpd 609 | 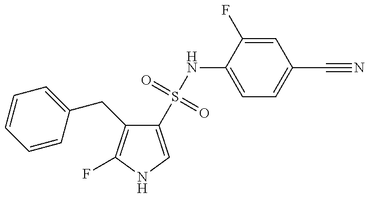 |
| Cpd 610 | 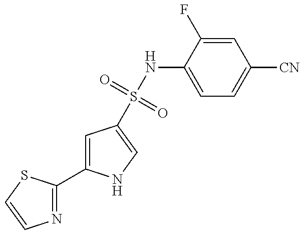 |
| Cpd 611 | 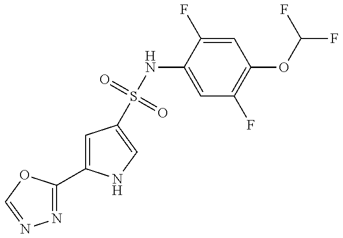 |
| Cpd 612 | 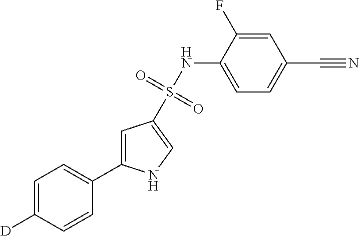 |
| Cpd 613 | 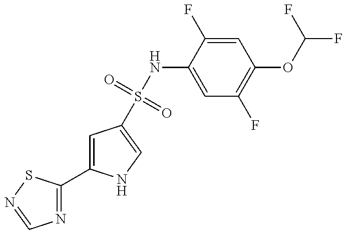 |
| Cpd 614 | 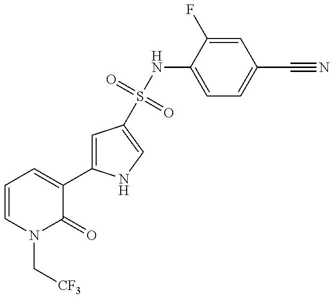 |
| Cpd 615 | 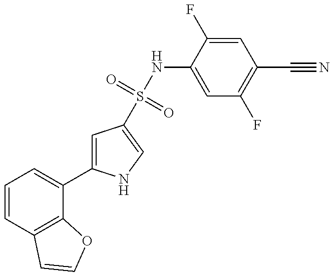 |
| Cpd 616 | 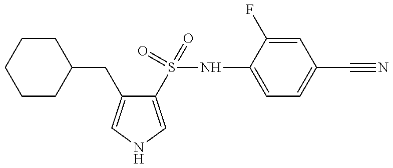 |
| Cpd 617 | 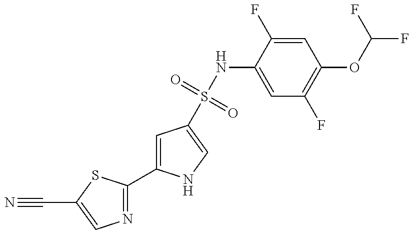 |
| Cpd 618 | 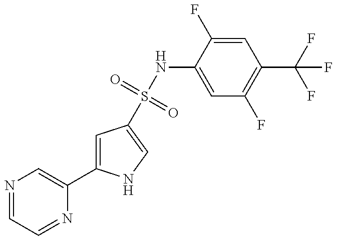 |
| Cpd 619 | 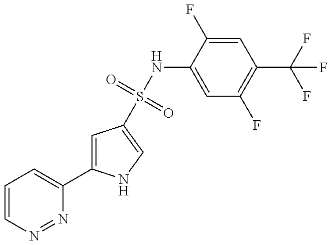 |
| Cpd 620 | 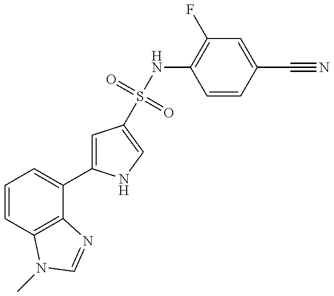 |
| Cpd 621 | 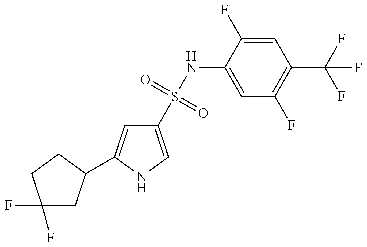 |
| Cpd 622 | 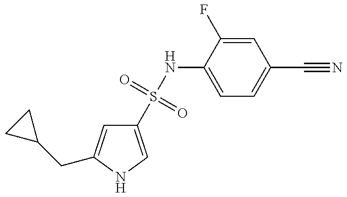 |
| Cpd 623 | 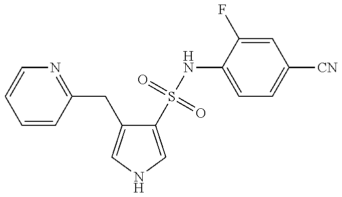 |
| Cpd 624 | 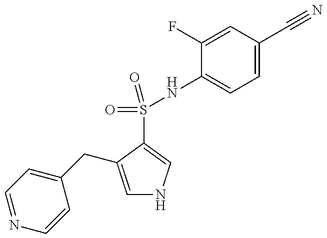 |
| Cpd 625 | 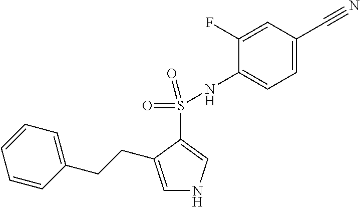 |
| Cpd 626 | 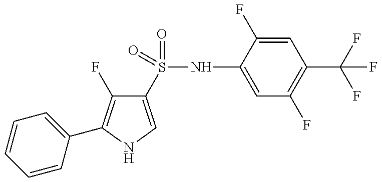 |
| Cpd 627 | 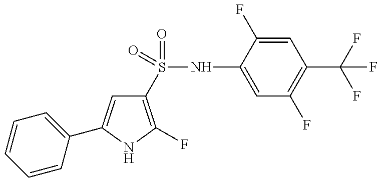 |
| Cpd 628 | 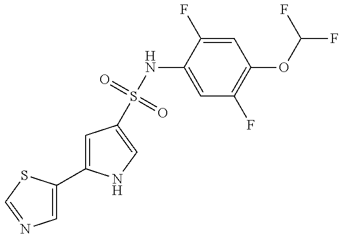 |
| Cpd 629 | 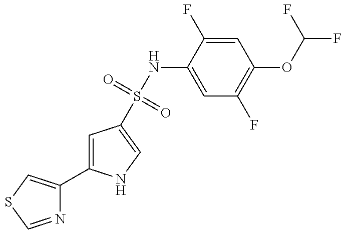 |
| Cpd 630 | 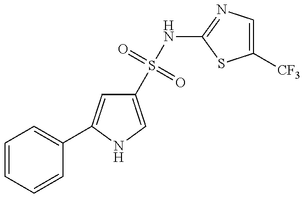 |
| Cpd 631 | 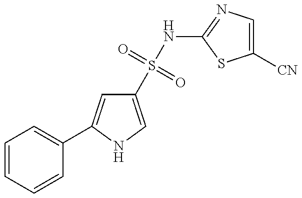 |
| Cpd 632 | 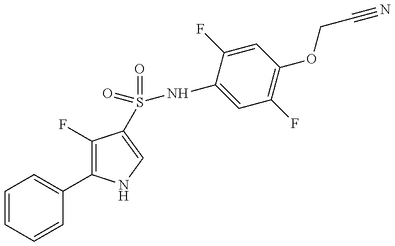 |
| Cpd 633 | 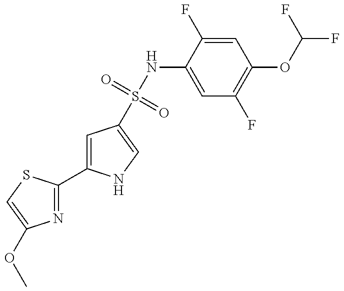 |
| Cpd 634 | 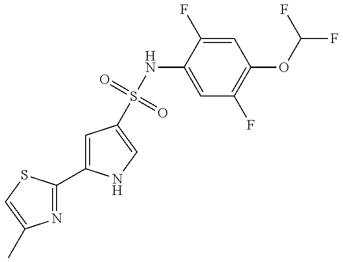 |
| Cpd 635 | 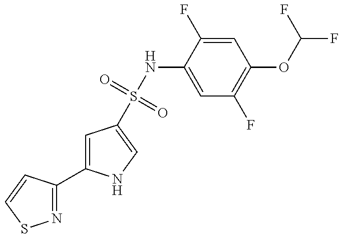 |
| Cpd 636 | 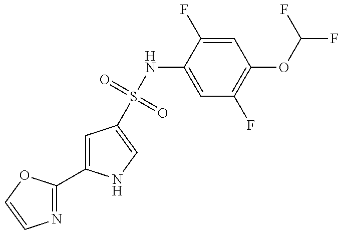 |
| Cpd 637 | 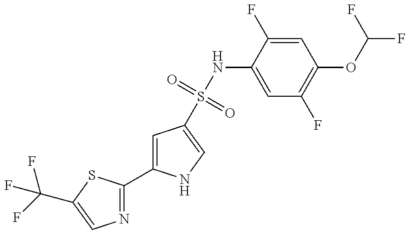 |
| Cpd 638 | 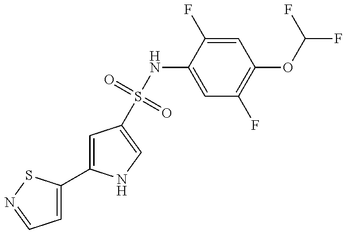 |
| Cpd 639 | 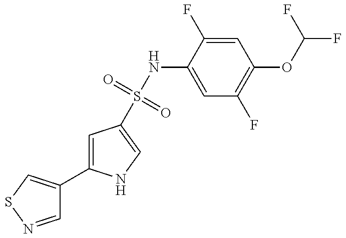 |
| Cpd 640 | 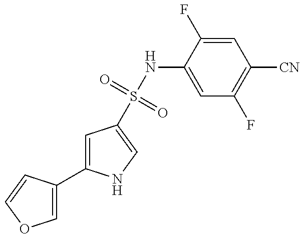 |
| Cpd 641 | 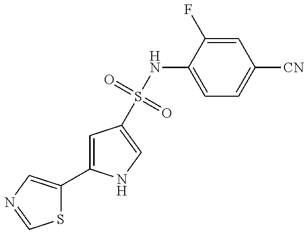 |
| Cpd 642 | 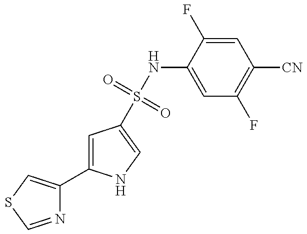 |
| Cpd 643 | 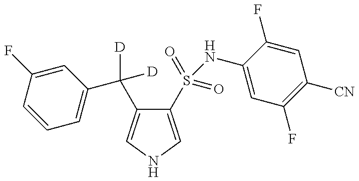 |
| Cpd 644 | 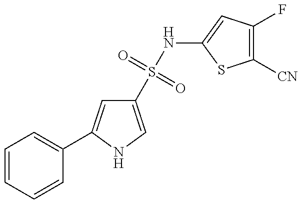 |
| Cpd 645 | 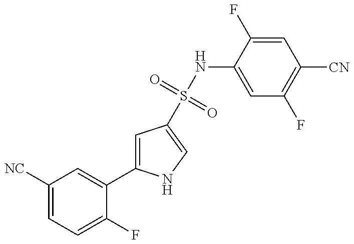 |
| Cpd 646 | 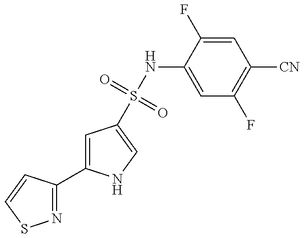 |
| Cpd 647 | 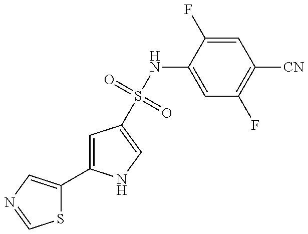 |
| Cpd 648 | 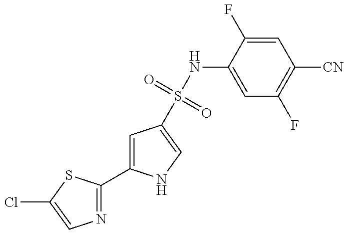 |
| Cpd 649 | 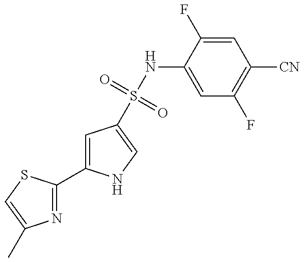 |
| Cpd 650 | 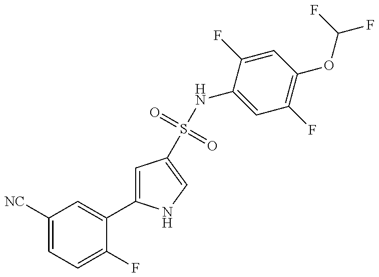 |
| Cpd 651 | 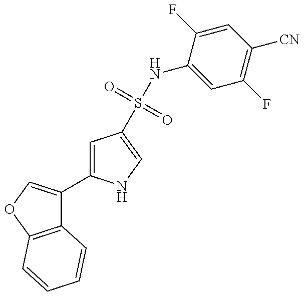 |
| Cpd 652 | 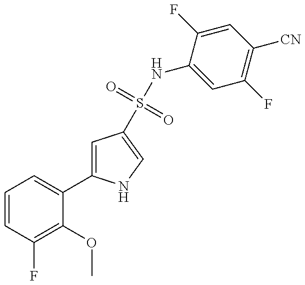 |
| Cpd 653 | 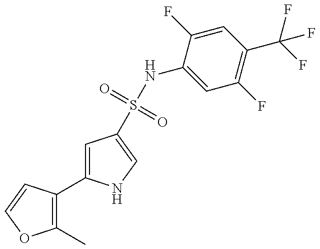 |
| Cpd 654 | 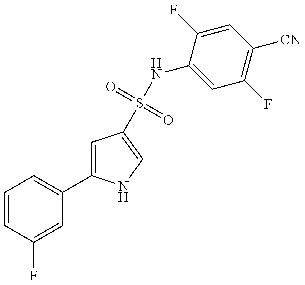 |
| Cpd 655 | 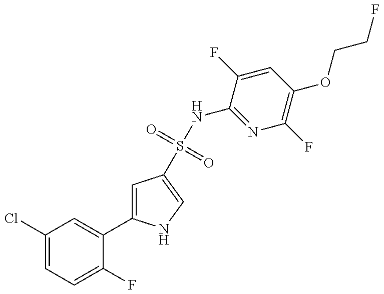 |
| Cpd 656 | 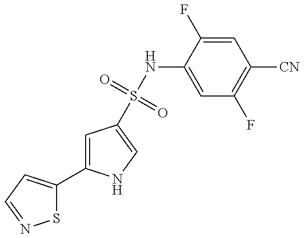 |
| Cpd 657 | 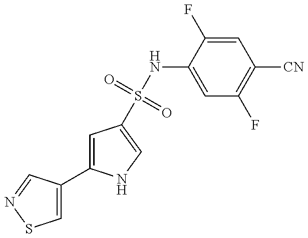 |
| Cpd 658 | 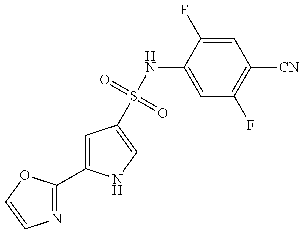 |
| Cpd 659 | 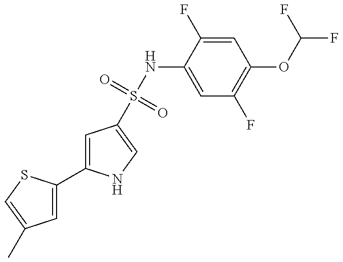 |
| Cpd 660 | 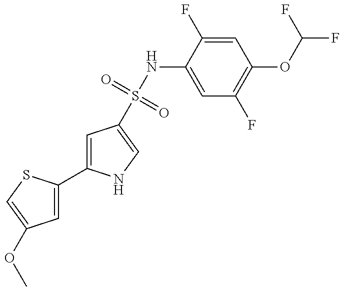 |
| Cpd 661 | 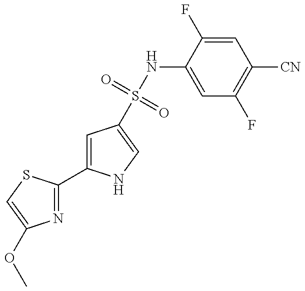 |
| Cpd 662 | 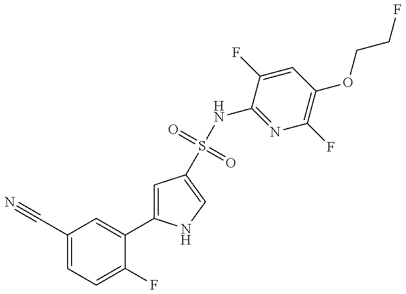 |
| Cpd 663 | 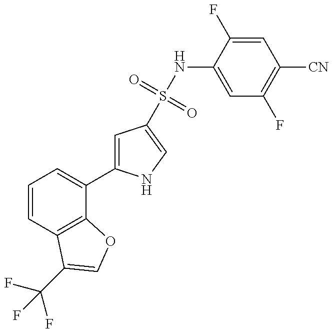 |
| Cpd 664 | 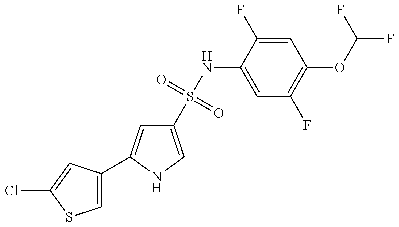 |
| Cpd 665 | 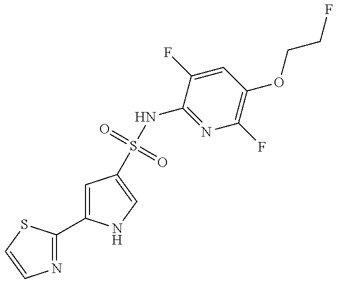 |
| Cpd 666 | 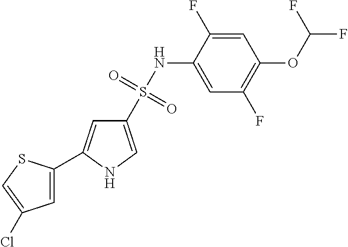 |
| Cpd 667 | 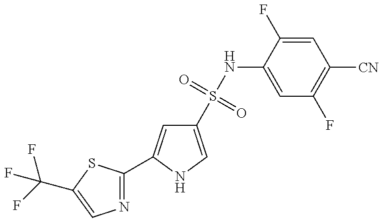 |
| Cpd 668 | 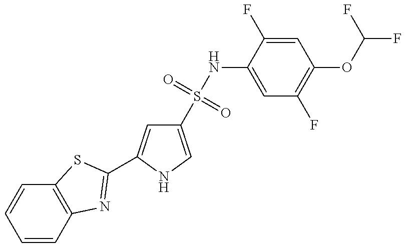 |
| Cpd 669 | 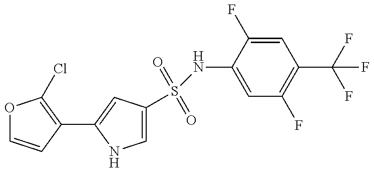 |

Part A represents the preparation of the compounds (intermediates and final compounds) whereas Part B represents the pharmacological examples.

### Part A

All starting materials which are not explicitly described were either commercially available (the details of suppliers such as for example Aldrich, Combi-Blocks, Enamine, FluoroChem, MatrixScientific, Merck, TCI, etc. can be found in the SciFinder® Database for example) or the synthesis thereof has already been described precisely in the specialist literature (experimental guidelines can be found in the Reaxys® Database or the SciFinder® Database respectively, for example) or can be prepared using the conventional methods known to the person skilled in the art.

The reactions were, if necessary, carried out under an inert atmosphere (mostly argon and N₂).

The number of equivalents of reagents and the amounts of solvents employed as well as the reaction temperatures and times can vary slightly between different reactions carried out by analogous methods. The work-up and purification methods were adapted according to the characteristic properties of each compound and can vary slightly for analogous methods. The yields of the compounds prepared are not optimized.

The LC/MS analyses mentioned in the experimental part were performed on a Waters system combining a Waters Acquity UPLC H-Class equipped with an Acquity UPLC PDA Detector and an Acquity TQ Detector (ESI).

The GCMS analyses mentioned in the experimental part were performed on an Agilent 7890B gas chromatography system coupled with 5977B MSD detector.

| Method | | | | | | Col |
|---|---|---|---|---|---|---|
| Code | Column | Mobile phase A | Mobile phase B | Gradient | Flow | T |
| LC-1 | Waters: BEH | 10 mM | CH₃CN | From 100% A to 52% in 3.18 min. to | 0.5 | 40° C. |
| | C18 (1.7 μm, | CH₃COONH₄ in | | 10% A in 0.82 min. held for 1 min. | mL/min | |
| | 2.1*50 mm) | H₂O (pH 7)/ CH₃CN | | | | |
| | | (95/5). | | | | |
| LC-2 | Waters: BEH | 10 mM | CH₃CN | From 84% A to 42% in 3.4 min. to | 0.5 | 40° C. |
| | C18 (1.7 μm, | CH₃COONH₄ in | | 10% A in 0.6 min. held for 1 min. | mL/min | |
| | 2.1*50 mm) | H₂O (pH 7)/ CH₃CN | | | | |
| | | (95/5). | | | | |
| LC-3 | Waters: BEH | 10 mM | CH₃CN | From 52% A to 10% in 3.5 min. held | 0.5 | 40° C. |
| | C18 (1.7 μm, | CH₃COONH₄ in | | for 1.5 min. | mL/min | |
| | 2.1*50 mm) | H₂O (pH 7)/ CH₃CN | | | | |
| | | (95/5). | | | | |
| LC-4 | Agilent: | Water/ 0.1% Formic | CH₃CN/ 0.1% | From 5% B to 100% in 2.0 min. held | 1.5 | 40° C. |
| | Poroshell 120, | Acid | Formic Acid | for 0.7 min. | mL/min | |
| | EC-C18 (1.9 | | | | | |
| | μm, 3.0*30 mm) | | | | | |
| LC-5 | Waters: BEH C8 | Water/ 0.05% | 0.05% HCOOH | 95% A for 0.75 min. From 95% A to | 0.8 | 50° C. |
| | (1.7 μm, 2.1*50 | Formic Acid | in CH₃CN:Water | 75% A in 0.75 min, further to 5% A in | mL/min | |
| | mm) | | (90:10) | 1.50 min. held for 1 min. back to 95% | | |
| | | | | A in 0.60 min. held for 0.50 min. | | |
| LC-6 | Waters: BEH C8 | Water/ 0.05% | 0.05% HCOOH | 95% A for 1 min. From 95% A to 50% | 0.8 | 50° C. |
| | (1.7 μm, 2.1*50 | Formic Acid | in CH₃CN:Water | in 4 min. to 10% A in 3 min., held for | mL/min | |
| | mm) | | (90:10) | 2 min. Back to 95% A in 1.50 min. | | |
| | | | | held for 0.5 min. | | |
| LC-7 | Waters: BEH | 10 mM NH₄OAc in | 10 mM | 98% A for 0.75 min., From 98% A to | 0.5 | 50° C. |
| | C18 | H₂O | NH₄OAc in | 2% in 2.75 min., held for 1 min. Back | mL/min | |
| | (1.7 μm, 2.1*30 | | CH₃CN:Water | to 98% A in 0.25 min. held for 0.25 | | |
| | mm) | | (90:10) | min. | | |
| LC-8 | Waters: BEH | 5 mM NH₄OAc in | 5 mM NH₄OAc | 98% A for 1 min., From 98% A to | 0.5 | 50° C. |
| | C18 | H₂O | in CH₃CN:Water | 50% in 4 min. to 10% in 3 min. held | mL/min | |
| | (1.7 μm, 2.1*30 | | (90:10) | for 2 min. Back to 98% A in 2 min. | | |
| | mm) | | | held for 0.10 min. | | |
| LC-9 | Waters: BEH | Water /0.05% TFA | 0.05% TFA in | 95% A for 1 min., From 95% A to | 0.8 | 50° C. |
| | C18 | | CH₃CN:water | 50% in 4 min. to 10% in 3 min. held | mL/min | |
| | (1.7 μm, 2.1*60 | | (90:10) | for 2 min. Back to 95% A in 2 min. | | |
| | mm) | | | held for 0.10 min. | | |
| LC-10 | Agilent: Eclipse | Water/0.05% TFA | 0.05% TFA in | 95% A for 0.75 min. From 95% A to | 0.8 | 50° C. |
| | Plus RRHD C18 | | CH₃CN:Water | 75% A in 0.75 min. to 5% A in 1.50 | mL/min | |
| | (1.8 μm, 3.0*50 | | (90:10) | min. held for 1 min. back to 95% A in | | |
| | mm) | | | 0.50 min. | | |
| | | | | held for | | |
| | | | | 0.60 min. | | |
| LC-11 | Agilent: Eclipse | Water/0.05% | 0.05% HCOOH | 98% A for 1 min., From 98% A to | 0.8 | 50° C. |
| | Plus RRHD C18 | Formic Acid | in CH₃CN:Water | 50% in 4 min. to 10% in 3 min. held | mL/min | |
| | (1.8 μm, 3.0*50 | | (90:10) | for 2 min. Back to 98% A in 1.50 min. | | |
| | mm) | | | held for 0.50 min. | | |
| LC-12 | Halo: 90A C18 | Water/0.1% Formic | CH₃CN / 0.1% | From 5% B to 60% in 2.0 min. to | 1.5 | 40° C. |
| | (2.0 μm, 3.0*30 | Acid | Formic Acid | 100% in 0.3 min. held for 0.3 min. | mL/min | |
| | mm) | | | | | |
| LC-13 | Halo: 90A C18 | Water / 0.1% | CH₃CN / 0.1% | From 5% B to 100% in 2.0 min. held | 1.5 | 40° C. |
| | (2.0 μm, 3.0*30 | Formic Acid | Formic Acid | for 0.7 min. | mL/min | |
| | mm) | | | | | |
| LC-14 | Halo: 90A C18 | Water / 0.1% | CH₃CN / 0.1% | From 5% B to 65% in 1.9 min. to | 1.5 | 40°° C. |
| | (2.0 μm, 3.0*30 | Formic Acid | Formic Acid | 100% in 0.4 min. held for 0.35 min. | mL/min | |
| | mm) | | | | | |
| LC-15 | Halo: 90A C18 | Water / 0.1% | CH₃CN / 0.1% | From 5% B to 40% in 1.6 min. to | 1.5 | 40° C. |
| | (2.0 μm, 3.0*30 | Formic Acid | Formic Acid | 100% in 0.7 min. held for 0.4 min. | mL/min | |
| | mm) | | | | | |
| LC-16 | Halo: 90A C18 | Water / 0.1% | CH₃CN / 0.1% | From 5% B to 55% in 1.7 min. to | 1.5 | 40° C. |
| | (2.0 μm, 3.0*30 | Formic Acid | Formic Acid | 100% in 0.7 min. held for 0.3 min. | mL/min | |
| | mm) | | | | | |
| LC-17 | Halo: 90A C18 | Water / 0.1% | CH₃CN / 0.1% | From 5% B to 70% in 2.0 min. to | 1.5 | 40° C. |
| | (2.0 μm, 3.0*30 | Formic Acid | Formic Acid | 100% in 0.3 min. held for 0.44 min. | mL/min | |
| | mm) | | | | | |
| LC-18 | Halo: 90A C18 | Water / 0.1% | CH₃CN / 0.1% | From 30% B to 80% in 2.0 min. to | 1.5 | 40°° C. |
| | (2.0 μm, 3.0*30 | Formic Acid | Formic Acid | 100% in 0.3 min. held for 0.44 min. | mL/min | |
| | mm | | | | | |
| LC-19 | Halo: 90A C18 | Water / 0.1% | CH₃CN / 0.1% | From | 1.5 | 40° C. |
| | (2.7 μm, 3.0*50 | Formic Acid | Formic Acid | 5% B to 70% in 2.0 min. to 95% | mL/min | |
| | mm) | | | in 0.2 min. held for 0.5 min. | | |
| LC-20 | Agilent: | Water / 5 mM | CH₃CN | From 10% B to 70% in 2.0 min. to | 1.2 | 40° C. |
| | Poroshell HPH- | NH₄HCO₃ | | 95% in 0.2 min. held for 0.5 min. | mL/min | |
| | C18 (2.7 μm, | | | | | |
| | 3.0*50 mm) | | | | | |
| LC-21 | Agilent: | Water / 0.04% | CH₃CN | From 10% B to 50% in 1.9 min. to | 1.2 | 40°° C. |
| | Poroshell HPH- | NH₃H₂O | | 95% in 0.2 min. held for 0.6 min. | mL/min | |
| | C18 (2.7 μm, | | | | | |
| | 3.0*50 mm) | | | | | |
| LC-22 | Halo: 90A C18 | Water / 0.1% | CH₃CN / 0.1% | From 5% B to 50% in 1.6 min. to | 1.5 | 40° C. |
| | (2.0 μm, 3.0*30 | Formic Acid | Formic Acid | 100% in 0.7 min. held for 0.4 min. | mL/min | |
| | mm) | | | | | |
| LC-23 | Halo: 90A C18 | Water / 0.1% | CH₃CN / 0.1% | From 30% B to 70% in 2.0 min. to | 1.5 | 40°° C. |
| | (2.0 μm, 3.0*30 | Formic Acid | Formic Acid | 100% in 0.3 min. held for 0.44 min. | mL/min | |
| | mm) | | | | | |
| LC-24 | Kromasil: | Water / 5 mM | CH₃CN | From 30% B to 80% in 2.0 min. to | 1.0 | 40°° C. |
| | EternityShell- | NH₄HCO₃ | | 95% in 0.1 min. held for 0.7 min. | mL/min | |
| | C18 (2.5 μm, | | | | | |
| | 2.1*50 mm) | | | | | |
| LC-25 | Halo: 90A C18 | Water / 0.05% TFA | CH₃CN / 0.05% | From 30% B to 100% in 2.0 min. held | 1.5 | 40° C. |
| | (2.0 pm, 3.0*30 | | TFA | for 0.7 min. | mL/min | |
| | mm | | | | | |
| LC-26 | Halo: 90A C18 | Water / 0.1% | CH₃CN / 0.1% | From 20% B to 70% in 2.0 min. to | 1.5 | 40° C. |
| | (2.0 μm, 3.0*30 | Formic Acid | Formic Acid | 100% in 0.2 min. held for 0.54 min. | mL/min | |
| | mm) | | | | | |
| LC-27 | Halo: 90A C18 | Water / 0.1% | CH₃CN / 0.1% | From 30% B to 80% in 2.0 min. to | 1.5 | 40° C. |
| | (2.0 μm, 3.0*30 | Formic Acid | Formic Acid | 100% in 0.2 min. held for 0.54 min. | mL/min | |
| | mm) | | | | | |
| LC-28 | Halo: 90A C18 | Water / 0.1% | CH₃CN / 0.1% | From 30% B to 70% in 2.0 min. to | 1.5 | 40° C. |
| | (2.0 μm, 3.0*30 | Formic Acid | Formic Acid | 100% in 0.2 min. held for 0.54 min. | mL/min | |
| | mm) | | | | | |
| LC-29 | Halo: 90A C18 | Water / 0.1% | CH₃CN / 0.1% | From 5% B to 60% in 2.2 min. to | 1.5 | 40° C. |
| | (2.0 μm, 3.0*30 | Formic Acid | Formic Acid | 100% in 0.3 min. held for 0.3 min. | mL/min | |
| | mm) | | | | | |
| LC-30 | Halo: 90A C18 | Water / 0.1% | CH₃CN / 0.1% | From 5% B to 100% in 2.1 min. held | 1.5 | 40° C. |
| | (2.7 μm, 3.0*50 | Formic Acid | Formic Acid | for 0.65 min. | mL/min | |
| | mm) | | | | | |
| LC-31 | SHIMADZU: | Water / 0.1% | CH₃CN / 0.1% | From 30% B to 70% in 1.8 min. to | 1.5 | 40° C. |
| | Shim-Pack | Formic Acid | Formic Acid | 100% in 0.2 min. held for 0.6 min. | mL/min | |
| | Scepter C18 | | | | | |
| | (3.0 μm, 3.0*33 | | | | | |
| | mm) | | | | | |
| LC-32 | Agilent: | Water / 5 mM | CH₃CN | From 10% B to 70% in 2.1 min. to | 1.5 | 40° C. |
| | Poroshell HPH- | NH₄HCO₃ | | 95% in 0.2 min. held for 0.3 min. | mL/min | |
| | C18 (4.0 μm, | | | | | |
| | 3.0*50 mm) | | | | | |
| LC-33 | Halo: 90A C18 | Water / 0.1% | CH₃CN / 0.1% | From 5% B to 70% in 1.8 min. to | 1.5 | 40° C. |
| | (2.0 μm, 3.0*30 | Formic Acid | Formic Acid | 100% in 0.2 min. held for 0.7 min. | mL/min | |
| | mm) | | | | | |
| LC-34 | Halo: 90A C18 | Water / 0.1% | CH₃CN / 0.1% | From 5% B to 60% in 2.2 min. to | 1.5 | 40° C. |
| | (2.0 μm, 3.0*30 | Formic Acid | Formic Acid | 100% in 0.3 min. held for 0.3 min. | mL/min | |
| | mm) | | | | | |
| LC-35 | Halo: 90A C18 | Water / 0.1% | CH₃CN / 0.1% | From 5% B to 80% in 1.9 min. to | 1.5 | 40° C. |
| | (2.0 μm, 3.0*30 | Formic Acid | Formic Acid | 100% in 0.1 min. held for 0.7 min. | mL/min | |
| | mm) | | | | | |

The MS analyses mentioned in the experimental part were performed on a Waters system combining a Waters Acquity UPLC H-Class equipped with an Acquity UPLC PDA Detector and an Acquity TQ Detector (ESI) by using UPLC in by-pass at 1 mL/min with 30% H₂O in CH₃CN as eluent.

### EXAMPLES OF THE PREPARATION OF INTERMEDIATES

### Synthesis of 4-cyclopropoxy-2,5-difluoroaniline (I-001)

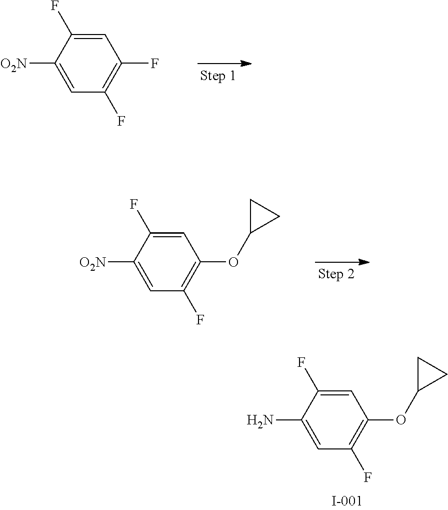 [SMILES: O=[N+]([O-])c1cc(F)c(F)cc1F ; O=[N+]([O-])c1cc(F)c(OC2CC2)cc1F ; Nc1cc(F)c(OC2CC2)cc1F]

Step 1: To a solution of 1,2,4-trifluoro-5-nitrobenzene (3.0 g, 16.9 mmol) and cyclopropanol (1.17 mL, 18.6 mmol) in DMF (60 mL), was added NaH (60% in mineral oil) (0.81 g, 20.2 mmol) at 0° C. The RM was stirred at RT. After 16 h, the RM was diluted with ice water and extracted with EtOAc. The organic phases were combined, washed with water, dried over Na₂SO₄, filtered, and concentrated under reduced pressure. The residue was purified by FCC on silica gel using a gradient of EtOAc (0-30%) in hexane to afford 2.5 g (69%) of (1-cyclopropoxy-2,5-difluoro-4-nitrobenzene. ¹H NMR (400 MHz, CDCl₃): δ ppm 7.89-7.80 (m, 1H), 7.20-7.15 (m, 1H), 3.90-3.84 (m, 1H), 0.92-0.91 (m, 4H).

Step 2: To a solution of 1-cyclopropoxy-2,5-difluoro-4-nitrobenzene (1.0 g, 4.6 mmol) in THF (50 mL) were added Fe powder (1.03 g, 18.6 mmol) and AcOH (2.79 mL, 46.5 mmol). The RM was heated at 80° C. for 5 h. The RM was filtered over celite bed. The filtrate was concentrated under reduced pressure. The residue was purified by FCC on silica gel using a gradient of EtOAc (0 to 30%) in hexane to afford 0.65 g (75%) of I-001. ¹H NMR (400 MHz, DMSO-d6): δ ppm 7.14-7.06 (m, 1H), 6.63-6.57 (m, 1H), 4.92 (s, 2H), 3.82-3.79 (m, 1H), 0.69-0.65 (m, 4H).

### Synthesis of bis(4-fluoro-2-methoxy-3-(4,4,5,5-tetramethyl-1,3,2-dioxaborolan-2-yl) pyridine) (I-002)

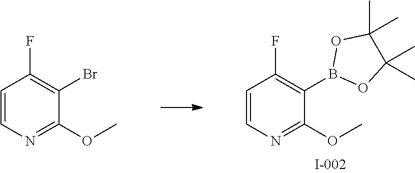

To a mixture of 3-bromo-4-fluoro-2-methoxypyridine (1.0 g, 2.4 mmol) and bis(pinacolato)diboron (1.23 g, 4.86 mmol) in dioxane (12 mL) and DMSO (0.6 mL) were added Pd(dppf)Cl₂ (0.18 g, 0.243 mmol) and AcOK (477.0 mg, 4.86 mmol) at RT under N₂. The RM was stirred for 3 h at 100° C. After cooling to RT, the RM was filtered. The solid was washed with EtOAc (3×30 mL). The filtrate was concentrated under reduced pressure to afford 1.4 g (40%) of I-002. LCMS (ES+, m/z) [M+H]⁺=254.1.

### Synthesis of 2-bromo-1-(2,2,2-trifluoroethyl) imidazole (I-003)

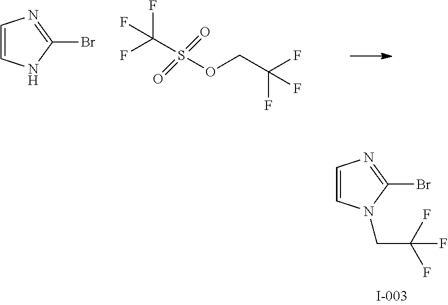

To a solution of 2-bromo-1H-imidazole (1 g, 6.80 mmol) in THF (30 mL) were added NaH (60% in mineral oil) (544 mg, 13.6 mmol) and 2,2,2-trifluoroethyl trifluoromethanesulfonate (1.58 g, 6.80 mmol) at RT under N₂. The RM was stirred for 3 h at RT. After cooling to RT, the RM was diluted with ice-water and extracted with DCM (3×100 mL). The organic phases were combined, washed with brine (3×50 mL), dried over Na₂SO₄, filtered, and concentrated under reduced. The residue was purified by FCC on silica gel using as eluent EtOAc/PE (1/3) to afford 1.3 g (82%) of (I-003). ¹H NMR (400 MHz, CDCl₃) δ 7.10 (s, 2H), 4.54 (m, 2H).

### Synthesis of 5-phenyl-1H-pyrrole-3-sulfonyl chloride (I-004)

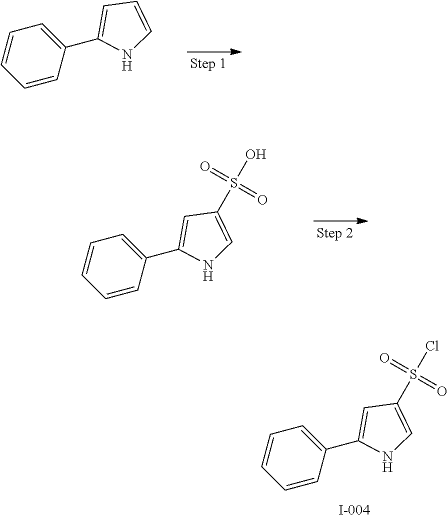 [SMILES: c1ccc(-c2ccc[nH]2)cc1 ; O=S(=O)(O)c1c[nH]c(-c2ccccc2)c1 ; O=S(=O)(Cl)c1c[nH]c(-c2ccccc2)c1]

Step 1: To a solution of 2-phenyl-1H-pyrrole (235 mg; 1.6 mmol) in MeCN (10 mL) was added Py·SO₃ (784 mg, 4.9 mmol). The RM was stirred for 3 h at 120° C. until completion. The RM was concentrated under reduced pressure. The residue was dissolved in water (50 mL) and washed with CHCl₃ (50 mL×3). The aqueous phase was concentrated under reduced pressure to afford 375 mg of 5-phenyl-1H-pyrrole-3-sulfonic acid.

Step 2: To a solution of 5-phenyl-1H-pyrrole-3-sulfonic acid (375 mg; 1.6 mmol) in MeCN (5 mL) was added was added POCl₃ (1.3 g, 8.4 mmol) at 0° C. The RM was stirred overnight at 70° C. The RM was poured into ice-water and extracted with CHCl₃ (3×50 mL). The combined organic layers were dried over Na₂SO₄, filtrated, and concentrated under reduced pressure to afford 535 mg of 5-phenyl-1H-pyrrole-3-sulfonyl chloride (I-004), which was used without further purification.

### Synthesis of 2-benzyl-1H-pyrrole (I-007)

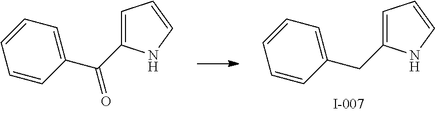 [SMILES: O=C(c1ccccc1)c1ccc[nH]1 ; c1ccc(Cc2ccc[nH]2)cc1]

To a solution of 2-benzoyl-1H-pyrrole (2.0 g, 11.7 mmol) in IPA (20 mL) was added NaBH₄ (880 mg, 23.4 mmol) in portions at 0° C. The RM was stirred overnight at 80° C. under nitrogen atmosphere. The reaction was quenched with ice-water at 0° C. The resulting mixture was diluted with water (100 mL), extracted with EtOAc (3×100 mL). The organic layers were combined, washed with brine (2×100 mL), dried over Na₂SO₄, filtrated, concentrated under reduced pressure. The residue was purified by R^{p} flash chromatography on C18 gel using a gradient of MeCN (50 to 65%) in water (0.1% NH₃HCO₃) to afford 800 mg (44%) of 2-benzyl-1H-pyrrole (I-007). ¹H NMR (300 MHz, DMSO-d6) δ ppm 10.64 (s, 1H), 7.35-7.13 (m, 5H), 6.64-6.61 (m, 1H), 5.96-5.92 (m, 1H), 5.82-5.74 (m, 1H), 3.89 (d, 2H).

### Synthesis of 2-fluoro-3-methyl-4-(trifluoromethyl)aniline (I-013)

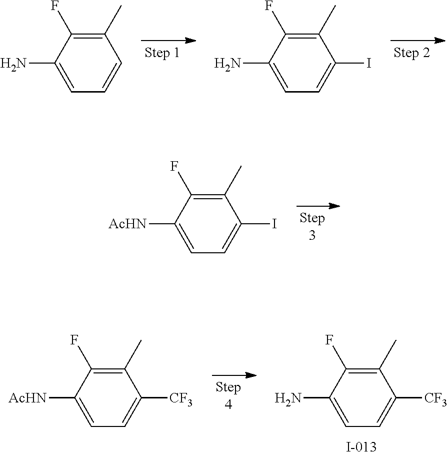

Step 1: NIS (3.60 g, 16.0 mmol) was added to a stirred solution of 2-fluoro-3-methylaniline (2 g, 16.0 mmol) in dry MeCN (20 mL) and the reaction mixture was stirred at RT. After 4 h solvent was removed under reduced pressure and the resulting crude was partitioned between ethyl acetate and water. Aqueous layer was further extracted with ethyl acetate. Organic layers were dried over Na₂SO₄, filtered and evaporated under reduced pressure. The residue was purified by FCC on silica gel using a gradient of EtOAc (0-40%) in hexane to afford 1.7 g (42%) of 2-fluoro-4-iodo-3-methylaniline. ¹H NMR (400 MHz, CDCl₃): δ ppm 7.32 (dd, 1H), 6.39 (t, 1H), 3.66 (bs, 2H), 2.30 (s, 3H).

Step 2: Triethyl amine (1.11 mL, 8.0 mmol) was added to a stirred solution of 2-fluoro-4-iodo-3-methylaniline (1 g, 4.0 mmol) in dry DCM (10 mL). RM was then cooled at 0° C. and was treated dropwise with acetyl chloride (0.34 mL, 4.8 mmol). Reaction mixture was allowed to warm up and stirred at RT. After 2 h, the reaction mixture was partitioned between DCM-water. Organic layer was dried over Na₂SO₄, filtered and evaporated under reduced pressure. The residue was purified by FCC on silica gel using a gradient of EtOAc (0-20%) in hexane to afford 940 mg (80%) of N-(2-fluoro-4-iodo-3-m ethylphenyl)acetamide (940 mg, 80%). ¹H NMR (400 MHz, CDCl₃): δ ppm 7.92 (t, 1H), 7.54 (d, 1H), 7.30 (s, 1H), 2.34 (d, 3H), 2.20 (s, 3H).

Step 3: HMPA (1.48 mL, 8.5 mmol), cuprous iodide (487.38 mg, 2.6 mmol) and methyl 2,2-difluoro-2-(fluorosulfonyl)acetate (1.09 mL, 8.5 mmol) were added to a stirred solution of N-(2-fluoro-4-iodo-3-methylphenyl)acetamide (500 mg, 1.7 mmol) in dry DMF (5 mL) at RT. The reaction mixture was then heated at 80° C. overnight. After completion of the reaction (monitored by LCMS), reaction mass was filtered through celite bed and was then diluted with EtOAc, washed with saturated aqueous NH₄Cl, dried over Na₂SO₄, filtered and concentrated under reduced pressure. The residue was purified by FCC on silica gel using a gradient of EtOAc (0-40%) in hexane to afford 280 mg (69%) of N-(2-fluoro-3-methyl-4-(trifluoromethyl)phenyl)acetamide that was uses in the next step without further purification.

Step 4: 6N HCl solution (2.6 mL) was added to a stirred solution of N-(2-fluoro-3-methyl-4-(trifluoromethyl)phenyl)acetamide (343.81 mg, 1.5 mmol) in ethanol (5 mL). Reaction mixture was then heated at reflux. After 2 h, solvent was evaporated under low temperature to obtain 240 mg (85%) of crude 2-fluoro-3-methyl-4-(trifluoromethyl)aniline (I-013) that was used for next step without further purification. ¹H NMR (400 MHz, DMSO-d6): δ ppm 7.16 (d, 1H), 6.66 (t, 1H), 2.23 (s, 3H).

### Synthesis of b-chloro-4-(difluoromethoxy)-2-fluoroaniline (I-014)

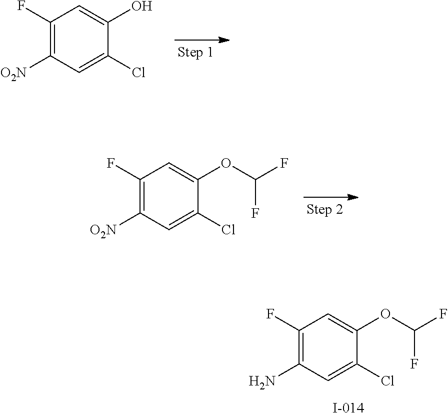 [SMILES: O=[N+]([O-])c1cc(Cl)c(O)cc1F ; O=[N+]([O-])c1cc(Cl)c(OC(F)F)cc1F ; Nc1cc(Cl)c(OC(F)F)cc1F]

Step 1: 2-chloro-5-fluoro-4-nitrophenol (1.1 g, 5.7 mmol) was taken in MeCN (20 mL) and the reaction mixture was cooled to 0° C. KOH (1.61 g, 28.7 mmol) was added and the reaction mixture was stirred at 0° C. for 30 min. After that diethyl (bromodifluoromethyl)phosphonate (5.11 g, 28.7 mmol) was added and reaction mixture was allowed to warm up and stirred at RT. After 16 h, reaction mixture was partitioned between DCM and water. Organic layer was separated, dried over Na₂SO₄, filtered and evaporated under reduced pressure. The residue was purified by FCC on silica gel using a gradient of EtOAc (0-3%) in hexane to afford 950 mg (68%) of 1-chloro-2-(difluoromethoxy)-4-fluoro-5-nitrobenzene. ¹H NMR (400 MHz, DMSO-d6): δ ppm 8.48 (d, 1H), 7.78 (d, 1H), 7.51 (t, 1H).

Step 2: To a stirred solution of 1-chloro-2-(difluoromethoxy)-4-fluoro-5-nitrobenzene (850 mg, 3.5 mmol) in Ethanol:Water (20:1, 42.0 mL) were added Fe powder (589.59 mg, 10.6 mmol) and CaCl₂ (390.56 mg, 3.5 mmol). Reaction mixture was then stirred at 80° C. After 16 hours, reaction mixture was filtered through a small bed of celite and the filtrate was evaporated under reduced pressure. The resulting crude was partitioned between ethyl acetate-water. Organic layer was separated, dried over Na₂SO₄, filtered and evaporated under reduced pressure. The residue was purified by FCC on silica gel using a gradient of EtOAc (0-10%) in hexane to afford 500 mg (67%) of 5-chloro-4-(difluoromethoxy)-2-fluoroaniline (I-014). ¹H NMR (400 MHz, DMSO-d6): δ ppm 7.16-7.13 (m, 1H), 7.02 (t, 1H), 6.89-6.87 (m, 1H), 5.46 (s, 2H).

### Synthesis of 4-(difluoromethoxy)-2-fluoro-5-methylaniline (I-015)

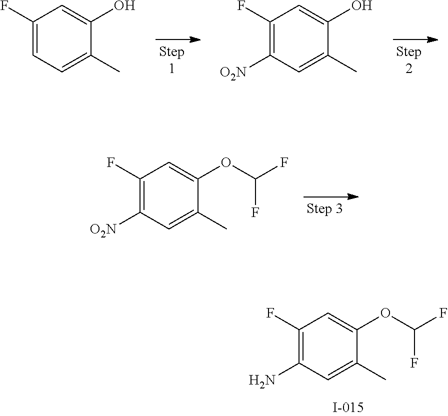

Step 1: To a solution of tert-butyl nitrite (1.5 mL, 12.7 mmol) in acetonitrile (20.0 mL) was added 5-fluoro-2-methylphenol (2 g, 15.8 mmol) and the reaction mixture was stirred at RT. After 12 hours, the reaction mixture was quenched with 5% aqueous sodium thiosulfate solution and extracted with ethyl acetate. Organic layer was separated, washed with water, brine, dried over Na₂SO₄, filtered and concentrated under reduced pressure. The residue was purified by FCC on silica gel using a gradient of EtOAc (0-20%) in Hexane to afford 550 mg (20%) of 5-fluoro-2-methyl-4-nitrophenol. ¹H NMR (400 MHz, DMSO-d6): δ ppm 11.48 (br, 1H), 7.97 (d, 1H), 6.76 (d, 1H), 2.13 (s, 3H).

Step 2: In a sealed tube, a solution of 5-fluoro-2-methyl-4-nitrophenol (550.0 mg, 3.2 mmol) and KOH (3.6 gm, 64.3 mmol) in a 1:1 mixture of MeCN (5.0 mL) and water (5.0 mL) was cooled to −78° C. Added diethyl (bromodifluoromethyl)phosphonate (1.14 mL, 6.4 mmol) in one portion, sealed the tube and the reaction mixture was allowed to warm up and stirred at RT. After 16 h, reaction mixture was diluted with water and extracted with ethyl acetate. Combined organic layers were washed with brine solution, dried over Na₂SO₄, filtered and concentrated under reduced pressure. The residue was purified by FCC on silica gel using a gradient of EtOAc (0 to 20%) in Hexane to obtain 250 mg (35%) of 1-(difluoromethoxy)-5-fluoro-2-methyl-4-nitrobenzene. ¹H NMR (400 MHz, CDCl₃): δ ppm 7.98 (d, 1H), 7.03 (d, 1H), 6.62 (t, 1H), 2.31 (s, 3H).

Step 3: To a suspension of 1-(difluoromethoxy)-5-fluoro-2-methyl-4-nitrobenzene (250 mg, 1.1 mol) in a mixture of EtOH (10.0 mL) and water (0.6 mL), Fe powder (190 mg, 3.4 mol) and CaCl₂ (125 mg, 1.1 mmol) were added. The resulting suspension was stirred at 60° C. After 12 h, the reaction mixture was filtered to remove the iron residues, which were washed with EtOAc (2×20 mL). The organic extracts were washed with H₂O (3×10 mL), brine (2×10 mL), and dried over Na₂SO₄, filtered and concentrated under reduced pressure. The residue was purified by FCC on silica gel using a gradient of EtOAc (0-20%) in hexane to afford 110 mg (51%) of 4-(difluoromethoxy)-2-fluoro-5-methylaniline (I-015). GCMS (EI, m/z)=191.1.

### Synthesis of 2-((6-amino-5-fluoropyridin-3-yl)oxy)acetonitrile (I-016)

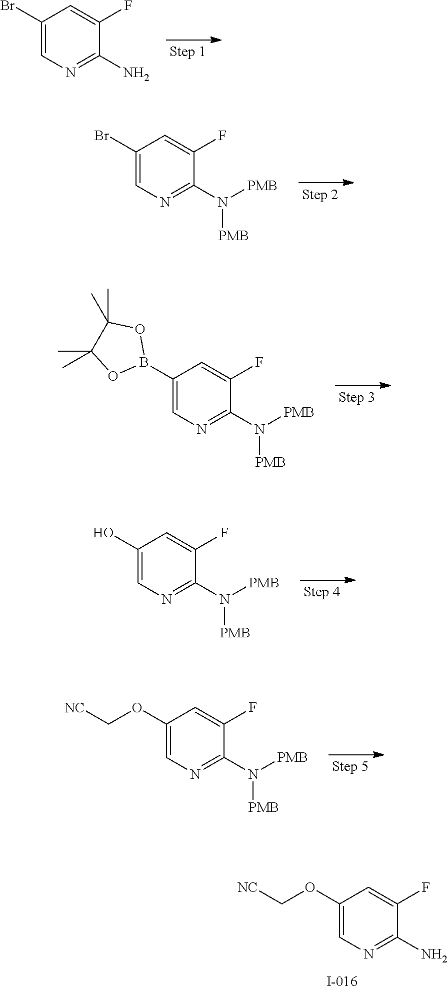

Step 1: To a stirred solution of 5-bromo-3-fluoropyridin-2-amine (2.0 g, 10.5 mmol) in DMAc (30.0 mL) was added NaH (60% dispersion in min. oil, 458 mg, 11.5 mmol) portion wise at 0° C. It was then stirred for 30 mins. PMB-Cl (4.26 mL, 31.4 mmol) was then added drop wise to it at 0° C.

The resulting solution was allowed to warm up and stirred at RT. After 2 hours, the reaction mixture was quenched with ice-cold water and extracted with ethyl acetate. Organic part was washed with water, brine, dried over anhydrous Na₂SO₄, filtered and concentrated under reduced pressure. The residue was purified by FCC on silica gel using a gradient of EtOAc (0-50%) in hexane to afford 2.37 g (52%) of 5-bromo-3-fluoro-N,N-bis(4-methoxybenzyl)pyridin-2-amine. LCMS (ES+, m/z) [M+H]⁺=430.9, 432.9.

Step 2: To a stirred solution of 5-bromo-3-fluoro-N,N-bis(4-methoxybenzyl)pyridin-2-amine (1.8 g, 4.2 mmol) in Dioxane (70.0 mL) were added Bis(pinacolato)diboron (2.12 g, 8.4 mmol) and AcOK (1.43 g, 14.6 mmol) at RT. Reaction mixture was degassed for 15 minutes with argon and Pd(dppf)Cl₂ (305 mg, 0.4 mmol) was added to the reaction mixture. The resulting reaction mixture was then heated at 100° C. After 16 hours, the reaction mixture was passed through celite bed and the filtrate was concentrated under reduced pressure to afford 1.9 g of 3-fluoro-N,N-bis(4-methoxybenzyl)-5-(4,4,5,5-tetramethyl-1,3,2-dioxaborolan-2-yl)pyridin-2-amine. Crude material was forwarded to the next step without further purification. LCMS (ES+, m/z) [M+H]⁺=479.0.

Step 3: To a stirred solution of 3-fluoro-N,N-bis(4-methoxybenzyl)-5-(4,4,5,5-tetramethyl-1,3,2-dioxaborolan-2-yl)pyridin-2-amine (1.9 g, 4.0 mmol) in THF (24.0 mL) was added H₂O₂ (30% in H₂O, 8 mL) at 0° C. The resulting reaction mixture was stirred 15 mins at 0° C. and then it was allowed to warm up and stirred at RT. After 2.5 hours, the reaction was quenched with aqueous NaHSO₃ and the aqueous mixture was extracted with ethyl acetate. Combined organic layers were then washed with brine, dried over anhydrous Na₂SO₄, filtered and concentrated under reduced pressure. The residue was purified by FCC on silica gel using a gradient of EtOAc (0-50%) in hexane to afford 1.37 g (94%) of 6-(bis(4-methoxybenzyl)amino)-5-fluoropyridin-3-ol. LCMS (ES+, m/z) [M+H]⁺=369.2. ¹H NMR (400 MHz, DMSO-d6): δ ppm 9.57 (s, 1H), 7.57 (s, 1H), 7.12 (d, 4H), 7.03-6.99 (m, 1H), 6.83 (d, 4H), 4.30 (s, 4H), 3.70 (s, 6H).

Step 4: To a stirred solution of 6-(bis(4-methoxybenzyl)amino)-5-fluoropyridin-3-ol (1.37 g, 3.7 mmol) in DMF (20.0 mL) was added K₂CO₃ (1.02 g, 7.4 mmol) at RT. Bromoacetonitrile (0.31 mL, 4.4 mmol) was then added drop wise at 0° C. to the reaction mixture. The resulting reaction mixture was allowed to warm up and was stirred at RT. After 16 h, the reaction mixture was diluted with ethyl acetate and washed with ice-cold water. Organic layer was then washed with water, brine, dried over anhydrous Na₂SO₄, filtered and concentrated under reduced pressure. The residue was purified by FCC on silica gel using a gradient of EtOAc (0-60%) in hexane to afford 800 mg (53%) of 2-((6-(bis(4-methoxybenzyl)amino)-5-fluoropyridin-3-yl)oxy)acetonitrile. LCMS (ES+, m/z) [M+H]⁺=408.3. ¹H NMR (400 MHz, DMSO-d6): δ ppm 7.87-7.86 (m, 1H), 7.54-7.50 (m, 1H), 7.15 (d, 4H), 6.85 (d, 4H), 5.15 (s, 2H), 4.46 (s, 4H), 3.71 (s, 6H).

Step 5: 2-((6-(bis(4-methoxybenzyl)amino)-5-fluoropyridin-3-yl)oxy)acetonitrile (800 mg, 2.0 mmol) was treated with TFA (10.0 mL) at 0° C. Reaction mixture was then left under stirring at RT. After 16 hours, the reaction mixture was concentrated under reduced pressure and the crude thus obtained was basified with aqueous NaHCO₃ solution. Aqueous phase was extracted with ethyl acetate for several times and then the combined organic part was washed with brine, dried over anhydrous Na₂SO₄, filtered and concentrated under reduced pressure to afford 300 mg (92%) of 2-((6-amino-5-fluoropyridin-3-yl)oxy)acetonitrile (I-016). LCMS (ES+, m/z) [M+H]⁺=168.2. ¹H NMR (400 MHz, DMSO-d6): δ ppm 7.69-7.68 (m, 1H), 7.42-7.38 (m, 1H), 5.98 (br s, 2H), 5.08 (s, 2H).

### Synthesis of 2-(4-amino-2,5-difluorophenoxy)acetonitrile (I-017)

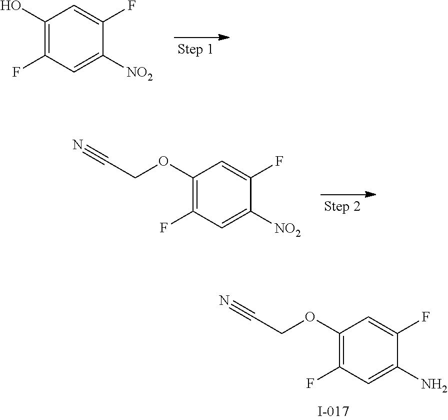 [SMILES: O=[N+]([O-])c1cc(F)c(O)cc1F ; N#CCOc1cc(F)c([N+](=O)[O-])cc1F ; N#CCOc1cc(F)c(N)cc1F]

Step 1: To a mixture of 2,5-difluoro-4-nitrophenol (700.0 mg, 3.998 mmol) in DMF (10.0 mL) was added K₂CO₃ (1103.43 mg, 7.996 mmol). The reaction mixture was cooled to 0° C., followed by slowly addition of Bromoacetonitrile (0.335 mL, 4.798 mmol). Reaction mixture was then stirred at RT for 16 hours. After completion, the reaction mixture was poured into cold water (30.0 mL) and extracted with ethyl acetate. Organic layer was separated, dried over anhydrous Na₂SO₄, filtered and concentrated under reduced pressure to obtain crude. Crude thus obtained was purified by FCC on silica gel using a gradient of EtOAc (5-40%) in hexane to afford 550 mg (64%) of 2-(2,5-difluoro-4-nitrophenoxy)acetonitrile. ¹H NMR (400 MHz, CDCl₃): δ ppm 7.99-7.95 (m, 1H), 7.01-6.97 (m, 1H), 4.94 (s, 2H).

Step 2: To a mixture of NH₄Cl (474.56 mg, 8.872 mmol) and Fe powder (297.24 mg, 5.323 mmol) in H₂O (4.0 mL) was added a solution of 2-(2,5-difluoro-4-nitrophenoxy)acetonitrile (380.0 mg, 1.774 mmol) in MeOH (5.0 mL). The reaction mixture was heated at 60° C. for 16 hours. Reaction mixture was filtered through celite bed and filtrate was concentrated under reduced pressure to obtain crude. Crude thus obtained was purified by FCC on silica gel using a gradient of EtOAc (5-40%) in hexane to afford 170 mg (52%) of 2-(4-amino-2,5-difluorophenoxy)acetonitrile (I-017). ¹H NMR (400 MHz, DMSO-d6): δ ppm 7.17-7.12 (m, 1H), 6.68-6.62 (m, 1H), 5.19 (brs, 2H), 5.04 (s, 2H).

### Synthesis of (4-amino-2,5-difluorophenyl)methanol (I-018)

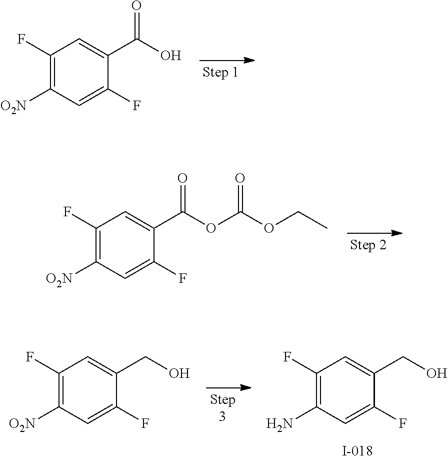 [SMILES: O=C(O)c1cc(F)c([N+](=O)[O-])cc1F ; CCOC(=O)OC(=O)c1cc(F)c([N+](=O)[O-])cc1F ; O=[N+]([O-])c1cc(F)c(CO)cc1F ; Nc1cc(F)c(CO)cc1F]

Step 1: To a stirred solution of 2,5-difluoro-4-nitrobenzoic acid (2.0 g, 9.8 mmol) in THF (8.0 mL) was added triethylamine (1.36 mL, 9.8 mmol) under argon atmosphere. The mixture was cooled to 0° C. and was treated with a solution of Ethyl chloroformate (1.03 mL, 10.8 mmol) in THF (12.0 mL) over 15 minutes. The reaction mixture was allowed to warm up and stirred at RT. After 16 h, the precipitate was filtered off and the filtrate was concentrated under reduced pressure to afford 2.0 g of crude (ethyl carbonic) 2,5-difluoro-4-nitrobenzoic anhydride that was used for the next step without further purification.

Step 2: To a stirred solution of (ethyl carbonic) 2,5-difluoro-4-nitrobenzoic anhydride (2.0 g, 7.3 mmol) in MeOH (12.0 mL) was added NaBH₄ (0.82 g, 21.8 mmol) at 0° C. portion wise. MeOH (6.0 mL) was added drop wise to the reaction mixture and reaction mixture was stirred at RT for 16 hours. The reaction mixture was acidified with aqueous 1N HCl and methanol was evaporated under reduced pressure. The residue was extracted with ethyl acetate. The organic phase was washed with saturated aqueous sodium bicarbonate solution and brine, dried over Na₂SO₄, filtered and concentrated under reduced pressure. to obtain crude. The residue was purified by FCC on silica gel using a gradient of EtOAc (10 to 45%) in hexane to afford 1.2 g (87%) of (2,5-difluoro-4-nitrophenyl)methanol. ¹H NMR (400 MHz, DMSO-d6): δ ppm 8.09-8.05 (m, 1H), 7.61-7.57 (m, 1H), 5.70 (t, 1H), 4.63 (d, 2H).

Step 3: To a stirred solution of (2,5-difluoro-4-nitrophenyl)methanol (700 mg, 3.7 mmol) in MeOH (10.0 mL) and water (9.0 mL), at RT, Zinc (12.10 g, 185.1 mmol) and NH₄Cl (1.58 g, 29.6 mmol) were added and the reaction mixture was stirred at RT. After 1 hour, the reaction mixture was filtered through celite and the filtrate was concentrated under reduced pressure. The residue was purified by FCC on silica gel using a gradient of EtOAc (10 to 60%) in hexane to afford 500 mg (85%) of (4-amino-2,5-difluorophenyl)methanol (I-018). ¹H NMR (400 MHz, DMSO-d6): δ ppm 6.99-6.95 (m, 1H), 6.50-6.45 (m, 1H), 5.31 (s, 2H), 4.99 (t, 1H), 4.33 (d, 2H).

### Synthesis of 5-(difluoromethoxy)-3-fluoropyridin-2-amine (I-019)

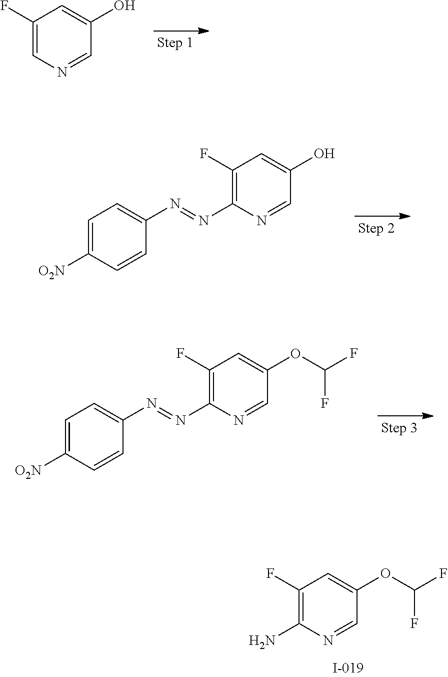 [SMILES: Oc1cncc(F)c1 ; O=[N+]([O-])c1ccc(/N=N/c2ncc(O)cc2F)cc1 ; O=[N+]([O-])c1ccc(/N=N/c2ncc(OC(F)F)cc2F)cc1 ; Nc1ncc(OC(F)F)cc1F]

Step 1: To a stirred mixture of p-nitroaniline (6.11 g, 44.2 mmol) and HCl (8.06 g, 221.06 mmol) in water (50 mL) was added NaNO₂ (3.05 g, 44.2 mmol) in small portions at 0° C. under nitrogen atmosphere. The resulting mixture was stirred for 1 h at 0° C. under nitrogen atmosphere. To the above mixture was added 5-fluoropyridin-3-ol (5 g, 44.2 mmol) and NaOH (10.61 g, 265.27 mmol) dropwise over 30 min at 0° C. The resulting mixture was stirred for additional 2 h at 0° C. The precipitated solids were collected by filtration and washed with water (3×100 mL). The residue was purified by FCC on silica gel using a gradient of EtOAc (0-20%) in hexane to afford 6.4 g (55%) of 5-fluoro-6-[(E)-2-(4-nitrophenyl) diazen-1-yl]pyridin-3-ol. ¹H NMR (400 MHz, CDCl₃) δ 7.93 (d, J=2.6 Hz, 2H), 7.25 (s, 1H), 7.15 (d, J=7.7 Hz, 1H), 7.05 (dd, J=8.5, 2.6 Hz, 2H), 5.76 (s, 1H).

Step 2: To a stirred mixture of 5-fluoro-6-[(E)-2-(4-nitrophenyl) diazen-1-yl]pyridin-3-ol (6.4 g, 24.4 mmol) and K₂CO₃ (16.87 g, 122.04 mmol) in DMF (50 mL) was added chlorodifluoromethane (6.33 g, 73.23 mmol) in small portions at 90° C. under nitrogen atmosphere. The resulting mixture was stirred for 16 h at 90° C. under nitrogen atmosphere. The mixture was allowed to cool down to RT and diluted with water (400 mL). The resulting mixture was extracted with EtOAc (3×300 mL). The combined organic layers were washed with brine (3×200 mL), dried over anhydrous Na₂SO₄. After filtration, the filtrate was concentrated under reduced pressure. The residue was purified by FCC on silica gel using a gradient of EtOAc (0-20%) in hexane to afford 1.4 g (18%) of 5-(difluoromethoxy)-3-fluoro-2-[(E)-2-(4-nitrophenyl) diazen-1-yl]pyridine. ¹H NMR (300 MHz, CDCl₃) δ 8.43 (m, 3H), 8.33-8.02 (m, 2H), 7.70-7.48 (m, 1H), 6.71 (m, 1H).

Step 3: To a stirred solution of 5-(difluoromethoxy)-3-fluoro-2-[(E)-2-(4-nitrophenyl) diazen-1-yl]pyridine (1.4 g, 4.48 mmol) in AcOH (20 mL) was added Pd/C (2.39 g, 22.42 mmol) in one portion at RT under hydrogen (60 atm) atmosphere. The resulting mixture was stirred for 24 h at 30° C. under hydrogen (60 atm) atmosphere. The mixture was allowed to cool down to RT. The resulting mixture was filtered, the filter cake was washed with ethyl acetate (3×50 mL). The filtrate was concentrated under reduced pressure. The residue was purified by FCC on silica gel using a gradient of EtOAc (0-20%) in hexane to afford 45 mg (6%) of 5-(difluoromethoxy)-3-fluoropyridin-2-amine (I-019). ¹H NMR (400 MHz, Methanol-d4) δ 7.68 (d, J=2.4 Hz, 1H), 7.29 (dd, J=11.2, 2.4 Hz, 1H), 6.69 (m, 1H).

### Synthesis of 2-(4-amino-2-chloro-5-fluorophenoxy)acetonitrile (I-020)

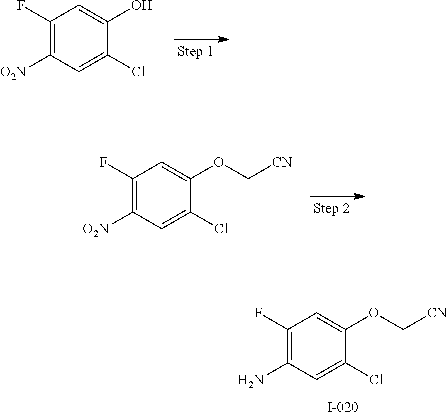 [SMILES: O=[N+]([O-])c1cc(Cl)c(O)cc1F ; N#CCOc1cc(F)c([N+](=O)[O-])cc1Cl ; N#CCOc1cc(F)c(N)cc1Cl]

Step 1: To a stirred solution of 2-chloro-5-fluoro-4-nitrophenol (1.96 g, 10.23 mmol) and K₂CO₃ (2.83 g, 20.46 mmol) in DMF (20 mL) was added 2-bromoacetonitrile (1.47 g, 12.28 mmol) dropwise at 0° C. under nitrogen atmosphere. The resulting mixture was stirred for 16 h at RT under nitrogen atmosphere. The resulting mixture was extracted with EtOAc (3×200 mL). The combined organic layers were washed with brine (3×60 mL), dried over anhydrous Na₂SO₄, filtered and concentrated under reduced pressure. The residue was purified by FCC on silica gel eluting with EtOAc/PE (1:5) to afford 840 mg (35%) of 2-(2-chloro-5-fluoro-4-nitrophenoxy) acetonitrile. ¹H NMR (400 MHz, CHCl₃) δ 8.25 (d, J=7.6 Hz, 1H), 6.96 (d, J=11.2 Hz, 1H), 4.96 (s, 2H).

Step 2: To a stirred mixture of 2-(2-chloro-5-fluoro-4-nitrophenoxy) acetonitrile (840 mg, 3.64 mmol) in MeOH (18 mL) and water (9 mL) were added NH₄Cl (1.94 g, 36.43 mmol) and Fe powder (1.01 g, 18.21 mmol) at RT under nitrogen atmosphere. The resulting mixture was stirred for 24 h at 50° C. under nitrogen atmosphere. The mixture was allowed to cool down to RT. The resulting mixture was filtered, the filter cake was washed with ethyl acetate (3×100 mL). The filtrate was concentrated under reduced pressure. The resulting mixture was extracted with EtOAc (3×150 mL). The combined organic layers were washed with brine (3×50 mL), dried over anhydrous Na₂SO₄, filtered and concentrated under reduced pressure. The residue was purified by FCC on silica gel eluting with EtOAc/PE (1:6) to afford 630 mg (85%) of 2-(4-amino-2-chloro-5-fluorophenoxy)acetonitrile (I-020). ¹H NMR (300 MHz, DMSO-d6) δ 7.19 (d, J=12.3 Hz, 1H), 6.88 (d, J=9.1 Hz, 1H), 5.20 (s, 2H), 5.10 (s, 2H).

### Synthesis of 2-(4-methoxythiophene-3-yl)-4,4,5,5-tetramethyl-1,3,2-dioxaborolane (I-021)

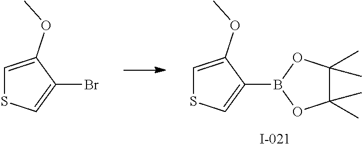

To a stirred solution of 3-bromo-4-methoxythiophene (150.00 mg, 0.8 mmol) in Dioxane (5.0 mL) were added AcOK (266.99 mg, 2.7 mmol) and bis pinacolato diboron (394.66 mg, 1.6 mmol) and the reaction mixture was degassed with Argon for 15-20 minutes. After that Pd(dppf)Cl₂ (56.87 mg, 0.08 mmol) was added and the reaction mixture was stirred at 100° C. After 16 hours, the reaction mixture was filtered through celite bed and the filtrate was concentrated under reduced pressure to afford crude 2-(4-methoxythiophen-3-yl)-4,4,5,5-tetramethyl-1,3,2-dioxaborolane (I 021) that was used for the next step without further purification.

### Synthesis of 1-bromo-2-cyclopropoxy-3-fluorobenzene (I-022)

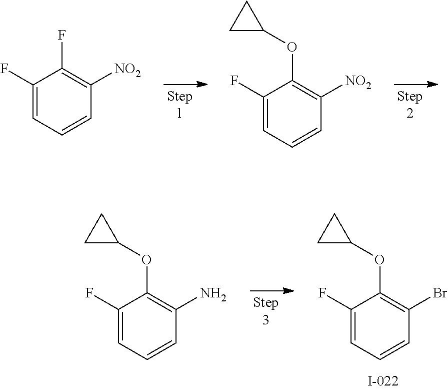 [SMILES: O=[N+]([O-])c1cccc(F)c1F ; O=[N+]([O-])c1cccc(F)c1OC1CC1 ; Nc1cccc(F)c1OC1CC1 ; Fc1cccc(Br)c1OC1CC1]

Step 1: In an oven-dried sealed tube was placed a mixture of 1,2-difluoro-3-nitrobenzene (1.0 g, 6.3 mmol) and CS₂CO₃ (3.07 g, 9.4 mmol) in DMF (20.0 mL). To the mixture, cyclopropanol (0.48 mL, 7.6 mmol) was added at RT. The resulting solution was stirred at 80° C. for 16 hours. The reaction mixture was diluted with ice-cold water and extracted with ethyl acetate. Organic phase was washed with brine, dried over Na₂SO₄, filtered and concentrated under low temperature and low pressure to afford 1.1 g of crude 2-cyclopropoxy-1-fluoro-3-nitrobenzene that was used in the next step without further purification. ¹H NMR (400 MHz, DMSO-d6): δ ppm 7.74-7.66 (m, 2H), 7.36-7.30 (m, 1H), 4.38-4.33 (m, 1H), 0.79-0.77 (m, 2H), 0.67-0.60 (m, 2H).

Step 2: To a stirred solution of 2-cyclopropoxy-1-fluoro-3-nitrobenzene (850 mg, 4.3 mmol) in ethanol (3.0 mL) was added Pd/C (450 mg, 10 wt %) at RT. Reaction mixture was left under stirring at RT under H₂-atmosphere. After 3 hours, the reaction mixture was passed through celite-bed and the filtrate was concentrated under reduced pressure. The residue was purified by FCC on silica gel using a gradient of EtOAc (0-10%) in hexane to afford 446 mg (62%) of 2-cyclopropoxy-3-fluoroaniline. ¹H NMR (400 MHz, DMSO-d6): δ ppm 6.78-6.73 (m, 1H), 6.46 (d, 1H), 6.36-6.31 (m, 1H), 5.08 (br s, 2H), 4.06-4.01 (m, 1H), 0.81-0.77 (m, 2H), 0.52-0.48 (m, 2H).

Step 3: To a stirred solution of 2-cyclopropoxy-3-fluoroaniline (380 mg, 2.3 mmol) in MeCN (15.0 mL) was added tBuONO (0.30 mL, 2.5 mmol) at 0° C. Cu(II)Br₂ (1.01 g, 4.6 mmol) was then added to the reaction mixture at same temperature. The resulting reaction mixture was left under stirring at 80° C. for 2 hours. Then, the reaction mixture was concentrated under low pressure and low temperature to afford 250 mg of crude 1-bromo-2-cyclopropoxy-3-fluorobenzene (I-022). GCMS (EI, m/z)=232.0.

### Synthesis of 2-bromo-3-(fluoromethyl)pyridine (I-023)

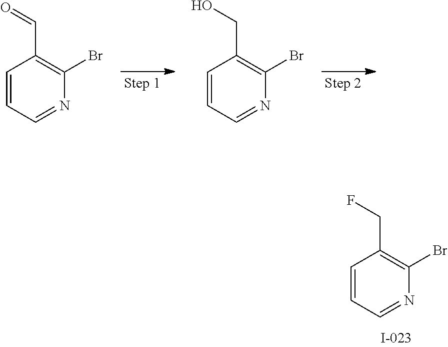 [SMILES: O=Cc1cccnc1Br ; OCc1cccnc1Br ; FCc1cccnc1Br]

Step 1: To a stirred solution of 2-bromonicotinaldehyde (2.0 g, 10.8 mmol) in MeOH (15.0 mL) was added NaBH₄ (0.45 g, 11.9) portion wise at 0° C. The reaction mixture was allowed to warm up and stirred at RT. After 16 hours, the reaction mixture was quenched with aqueous NH₄Cl and methanol was evaporated under reduced pressure. It was then diluted with water and extracted with ethyl acetate. Organic layer was washed with brine, dried over Na₂SO₄, filtered and concentrated under reduced pressure. The residue was purified by FCC on silica gel using a gradient of EtOAc (0-50%) in hexane to afford 1.9 g (93%) of (2-bromopyridin-3-yl)methanol. LCMS (ES+, m/z) [M+H]⁺=187.8, 189.8. ¹H NMR (400 MHz, DMSO-d6): δ ppm 8.27-8.25 (m, 1H), 7.89 (d, 1H), 7.48-745 (m, 1H), 5.58 (t, 1H), 4.49 (d, 2H).

Step 2: DAST (4.73 mL, 38.6 mmol) was added to a stirred solution of (2-bromopyridin-3-yl)methanol (1.9 g, 10.2 mmol) in dry DCM (20.0 mL) at 0° C. Reaction mixture was then stirred at RT for 3 hours. Reaction mixture was quenched with saturated NaHCO₃solution and the aqueous phase was extracted with DCM. Organic layer was dried over Na₂SO₄, filtered and evaporated under reduced pressure. The residue was purified by FCC on silica gel using a gradient of EtOAc (0-20%) in hexane to afford 570 mg (30%) of 2-bromo-3-(fluoromethyl)pyridine (I-023). ¹H NMR (400 MHz, DMSO-d6): δ ppm 8.41-8.40 (m, 1H), 7.96-7.94 (m, 1H), 7.56-7.52 (m, 1H), 5.56 (s, 1H), 5.46 (s, 1H).

### Synthesis of 5-bromo-4-fluorothiophene-2-carbonitrile (I-024)

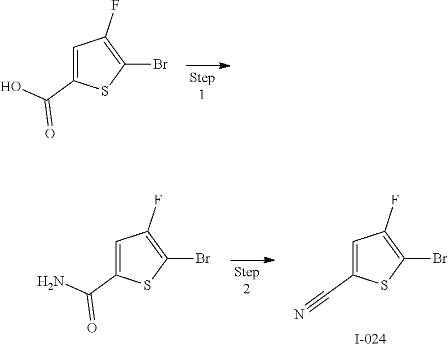 [SMILES: O=C(O)c1cc(F)c(Br)s1 ; NC(=O)c1cc(F)c(Br)s1 ; N#Cc1cc(F)c(Br)s1]

Step 1: To a stirred solution of 5-bromo-4-fluorothiophene-2-carboxylic acid (560.0 mg, 2.489 mmol) in DMF (10.0 mL) was added NH₄Cl (1232.58 mg, 24.886 mmol) and Et₃N (3.456 mL, 24.886 mmol) at 0° C. and reaction mixture was stirred at 0° C. for 5 minutes. Then EDC·HCl (1431.17 mg, 7.466 mmol) and HOBT (1008.76 mg, 7.466 mmol) were added and the reaction mixture was stirred at RT for 16 hours. After completion, reaction mixture was diluted with ice-cold water and extracted with ethyl acetate for several times. The organic phase was dried over anhydrous Na₂SO₄, filtered and concentrated under reduced pressure to get crude material. Crude thus obtained was purified by FCC on silica gel using a gradient of EtOAc (5-50%) in hexane to afford 280 mg (50%) of 5-bromo-4-fluorothiophene-2-carboxamide. ¹H NMR (400 MHz, DMSO): δ ppm 8.11 (s, 1H), 7.69 (s, 2H).

Step 2: To a stirred solution of 5-bromo-4-fluorothiophene-2-carboxamide (280.0 mg, 1.25 mmol) in DCM (5.0 mL) at −10° C. was added TFAA (0.191 mL, 1.375 mmol), followed by addition of Et₃N (0.382 mL, 2.749 mmol). The reaction mixture was allowed to warm up and was stirred at RT. After 4 hours, the reaction mixture was diluted with DCM (15.0 mL) and washed with saturated aqueous NaHCO₃ solution (10.0 mL) and then brine (10.0 mL). The organic phase was dried over anhydrous Na₂SO₄, filtered and concentrated under reduced pressure to obtain crude. Crude thus obtained was purified by FCC on silica gel using a gradient of EtOAc (0-20%) in hexane to afford 180 mg (70%) of 5-bromo-4-fluorothiophene-2-carbonitrile (I-024). ¹H NMR (400 MHz, DMSO-d6): δ ppm 8.07 (s, 1H).

The following compounds were prepared in a similar manner (use of appropriate reagents and purification methods (including chiral HPLC or chiral SFC) known to the person skilled in the art) as described for I-024: 5-bromo-3-fluorothiophene-2-carbonitrile (I-025).

### Synthesis of 2-(1H-pyrrol-2-yl)-1,3,4-oxadiazole (I-026)

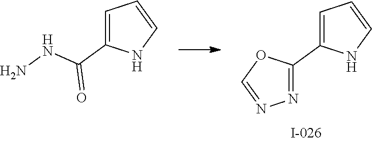 [SMILES: NNC(=O)c1ccc[nH]1 ; c1c[nH]c(-c2nnco2)c1]

A stirred solution of 1H-pyrrole-2-carbohydrazide (800 mg, 6.393 mmol) in Triethyl orthoformate (30.0 mL) was heated at 140° C. for 2 hours. After 2 hour, the reaction mixture was evaporated under reduced pressure to remove excess of triethyl orthoformate. Then, POCl₃ (10.0 mL) was added and the reaction mixture was heated at 100° C. for 30 mins. After completion, the reaction mixture was poured into crushed ice and extracted with ethyl acetate. The organic part was washed with water, dried over anhydrous Na₂SO₄, filtered and evaporated under reduced pressure. Crude thus obtained was purified by FCC on silica gel using a gradient of EtOAc (5-10%) in hexane to afford 663 mg (77%) of 2-(1H-pyrrol-2-yl)-1,3,4-oxadiazole (I-026). ¹H NMR (400 MHz, DMSO-d6): δ ppm 12.17 (s, 1H), 9.15 (s, 1H), 7.09-7.07 (m, 1H), 6.83-6.81 (s, 1H), 6.27-6.25 (m, 1H).

### Synthesis of 5-(1H-pyrrol-2-yl)-1,2,4-thiadiazole (I-027)

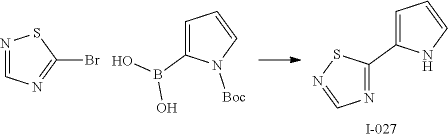

To a stirred solution of 5-bromo-1,2,4-thiadiazole (1.0 g, 6.06 mmol) in Dioxane (5.0 mL) was added (1-(tert-butoxycarbonyl)-1H-pyrrol-2-yl)boronic acid (1.726 g, 8.18 mmol). A solution of Na₂CO₃ (1.734 g, 16.36 mmol) in water (0.5 mL) was added to the reaction mixture and resulting mixture was degassed under argon for 15 minutes. Pd(PPh₃)₄ (630.27 mg, 0.545 mmol) was added to the reaction mixture under inert atmosphere and the reaction mixture was then heated at 80° C. for 16 hours. After completion, the volatiles were evaporated under reduced pressure and crude thus obtained was purified by FCC on silica gel using a gradient of EtOAc (0-60%) in hexane to afford 260 mg (32%) of 5-(1H-pyrrol-2-yl)-1,2,4-thiadiazole (I-027). ¹H NMR (400 MHz, DMSO-d6): δ ppm 12.16 (s, 1H), 8.70 (s, 1H), 7.12-7.10 (m, 1H), 6.97-6.95 (m, 1H), 6.28-6.26 (m, 1H).

### Synthesis of 5-(5-cyano-2-fluorophenyl)-1H-pyrrole-3-sulfonamide (I-028)

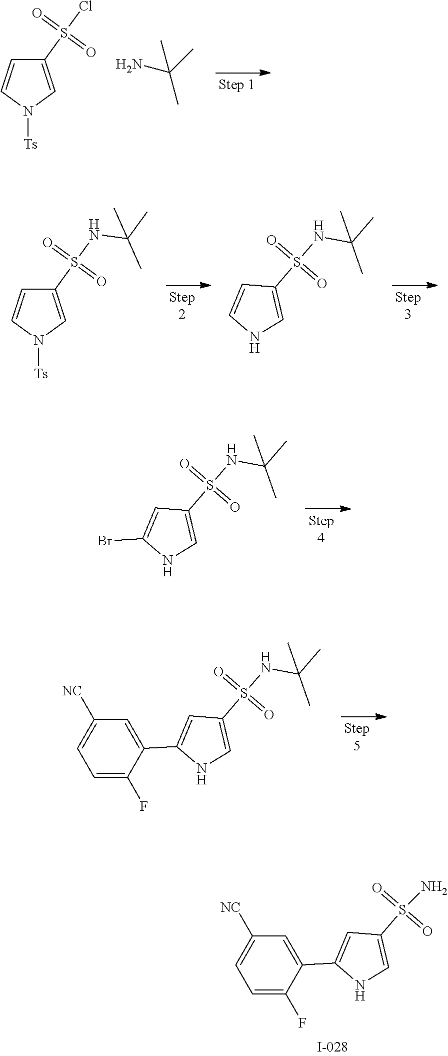

Step 1: To a stirred solution of 1-tosyl-1H-pyrrole-3-sulfonyl chloride (5.0 g, 15.64 mmol) in MeCN (20.0 mL) was added 2-methylpropan-2-amine (4.9 mL, 46.91 mmol) and pyridine (3.1 mL, 39.09 mmol). The reaction mixture was heated at 80° C. for 16 hours. Upon completion, reaction mixture was concentrated under reduced pressure and crude thus obtained was purified by FCC on silica gel using a gradient of EtOAc (0-60%) in hexane to afford 4.1 g (74%) of N-tert-butyl-1-[(4-methylbenzene)sulfonyl]-1H-pyrrole-3-sulfonamide. ¹H NMR (400 MHz, DMSO-d6): δ ppm 7.95 (d, 2H), 7.70-7.69 (m, 1H), 7.48-7.46 (m, 3H), 7.30 (s, 1H), 6.54-6.53 (m, 1H), 2.39 (s, 3H), 1.04 (s, 9H).

Step 2: To a stirred solution of N-(tert-butyl)-1-tosyl-1H-pyrrole-3-sulfonamide (4.1 g, 11.50 mmol) in MeOH (20.0 mL) was added a solution of LiOH·H₂O (2.41 g, 57.51 mmol) in water (10.0 mL). The reaction mixture was stirred for 1 h at RT. Upon completion, reaction mixture was concentrated under reduced pressure and the pH was adjusted to ˜7.0 with 2N aqueous HCl. Extracted the aqueous mixture with ethyl acetate. Organic phase was washed with brine, dried over anhydrous Na₂SO₄, filtered and concentrated under reduced pressure. Crude thus obtained was purified by FCC on silica gel using a gradient of EtOAc (0-50%) in hexane to afford 2 g (86%) of N-(tert-butyl)-1H-pyrrole-3-sulfonamide. ¹H NMR (400 MHz, DMSO-d6): δ ppm 11.34 (br s, 1H), 7.18 (s, 1H), 6.84-6.81 (m, 2H), 6.29-6.28 (m, 1H), 1.09 (s, 9H).

Step 3: To a stirred solution of N-(tert-butyl)-1H-pyrrole-3-sulfonamide (2 g, 9.89 mmol) in DMF (60.0 mL) was added NBS (1.58 g, 8.90 mmol) portion wise at 0° C. The reaction mixture was stirred at RT for 16 hours. Upon completion, reaction was diluted with ice-cold water and extracted with ethyl acetate. Organic phase was washed with brine, dried over anhydrous Na₂SO₄, filtered and concentrated under reduced pressure. Crude thus obtained was purified by FCC on silica gel using a gradient of EtOAc (0-60%) in hexane to afford 800 mg (29%) of 5-bromo-N-tert-butyl-1H-pyrrole-3-sulfonamide. ¹H NMR (400 MHz, DMSO-d6): δ ppm 12.13 (br s, 1H), 7.23 (s, 1H), 6.99 (s, 1H), 6.33 (s, 1H), 1.11 (s, 9H).

Step 4: To a stirred degassed solution of 5-bromo-N-tert-butyl-1H-pyrrole-3-sulfonamide (1.5 g, 5.34 mmol) in Dioxane/water (10:1, 11.0 mL) were added Na₂CO₃ (1.697 g, 16.01 mmol), (5-cyano-2-fluorophenyl)boronic acid (1.057 g, 6.41 mmol) and the reaction mixture was again degassed under argon. Pd(PPh₃)₄ (617 mg, 0.53 mmol) was then added to the reaction mixture under inert atmosphere and it was heated at 80° C. for 16 hours. After completion, the reaction mixture was filtered through a small celite pad and filtrate was evaporated. Crude thus obtained was purified by FCC on silica gel using a gradient of EtOAc (0-50%) in hexane to afford 1 g (58%) of N-(tert-butyl)-5-(5-cyano-2-fluorophenyl)-1H-pyrrole-3-sulfonamide. LCMS (ES−, m/z) [M−H]⁻=320.0.

Step 5: N-(tert-butyl)-5-(5-cyano-2-fluorophenyl)-1H-pyrrole-3-sulfonamide (1.0 g, 3.11 mmol) was taken in TEA (12.0 mL) at 0° C. and the reaction mixture was stirred at RT for 4 hours. After completion, reaction mixture was evaporated under reduced pressure, diluted with EtOAc and washed with saturated aqueous NaHCO₃ solution. Organic part was dried over anhydrous Na₂SO₄, filtered and concentrated under reduced pressure. Crude thus obtained was purified by FCC on silica gel using a gradient of EtOAc (0-70%) in hexane to afford 450 mg (54%) of 5-(5-cyano-2-fluorophenyl)-1H-pyrrole-3-sulfonamide (I-028). LCMS (ES−, m/z) [M−H]⁻=263.8.

### Synthesis of 4,4,5,5-tetramethyl-2-(3-(trifluoromethyl)benzofuran-7-yl)-1,3,2-dioxaborolane (I-029)

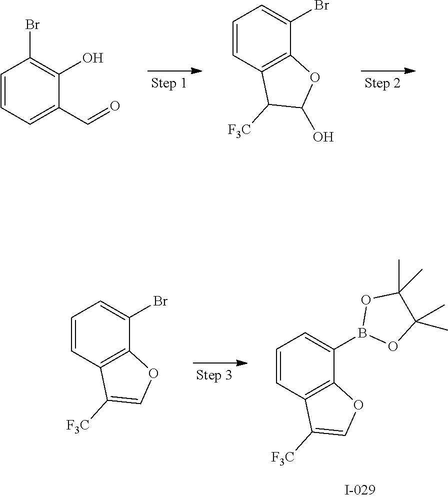 [SMILES: O=Cc1cccc(Br)c1O ; OC1Oc2c(Br)cccc2C1C(F)(F)F ; FC(F)(F)c1coc2c(Br)cccc12 ; CC1(C)OB(c2cccc3c(C(F)(F)F)coc23)OC1(C)C]

Step 1: To a stirred solution of 2,2,2-trifhioroethanamine·HCl (2.88 g, 21.29 mmol) in DCM (30.0 mL) at 0° C., was added a solution of sodium nitrite (1.56 g, 69.00 mmol) in water (3.0 mL). The mixture was kept at 0° C. for 1 hour. After that reaction mixture was cooled at −78° C. and methyl 3-bromo-5-formyl-4-hydroxybenzoate (0.5 g, 2.49 mmol) and BF₃·Et₂O (1.44 mL, 4.69 mmol) were added to the reaction mixture sequentially. After complete addition, reaction mixture was stirred at same temperature for 5 h and warmed to RT over a 12 hours period. After completion, the reaction was quenched with the addition of methanol (16.0 mL). The mixture was diluted with saturated aqueous NaHCO₃and the aqueous phase was extracted with ethyl acetate. Organic layer was washed with brine, dried over anhydrous Na₂SO₄, filtered and concentrated under reduced pressure. Crude thus obtained was purified by FCC on silica gel using a gradient of EtOAc (0-30%) in hexane to afford 363 mg (52%) of 7-bromo-3-(trifluoromethyl)-2,3-dihydrobenzofuran-2-ol. ¹H NMR (400 MHz, DMSO-d6): δ ppm 8.27 (d, 1H), 7.55 (d, 1H), 7.35 (d, 1H), 6.94 (t, 1H), 6.12-6.10 (m, 1H), 4.39-4.32 (m, 1H).

Step 2: A mixture of 7-bromo-3-(trifluoromethyl)-2,3-dihydrobenzofuran-2-ol (360 mg, 1.28 mmol) and sulfuric acid (4.584 mL, 85.55 mmol) was stirred at RT for 30 min. After completion, the reaction mixture was poured into ice/water (30.0 mL) and the white solid obtained was collected by filtration, dried in vacuum to provide 150 mg (44%) of 7-bromo-3-(trifluoromethyl)benzofuran which was used in the next step without further purification. ¹H NMR (400 MHz, CDCl₃): δ ppm 8.03 (s, 1H), 7.64 (d, 1H), 7.57 (d, 1H), 7.26-7.22 (m, 1H).

Step 3: To a degassed mixture of 7-bromo-3-(trifluoromethyl)benzofuran (150 mg, 0.566 mmol) in anhydrous dioxane (8.0 mL) were added bis(pinacolato)diboron (215 mg, 0.849 mmol), potassium acetate (166 mg, 1.68 mmol) and Pd(dppf)Cl₂·CH₂Cl₂ (46 mg, 0.057 mmol). The reaction mixture was heated at 100° C. for 16 hours in a sealed vial. After completion, reaction mixture was concentrated under reduced pressure and diluted with ethyl acetate (50.0 mL). Organic layer was washed with brine, dried over anhydrous Na₂SO₄, filtered and concentrated under reduced pressure. Crude thus obtained was purified by FCC on silica gel using a gradient of EtOAc (0-15%) in hexane to afford 150 mg (85%) of 4,4,5,5-tetramethyl-2-(3-(trifluoromethyl)benzofuran-7-yl)-1,3,2-dioxaborolane (I-029). NMR (400 MHz, CDCl₃): δ ppm 8.03 (br s, 1H), 7.84 (d, 1H), 7.79 (d, 1H), 1.40 (s, 12H).

### Synthesis of 2-(2-chlorofuran-3-yl)-4,4,5,5-tetramethyl-1,3,2-dioxaborolane (I-030)

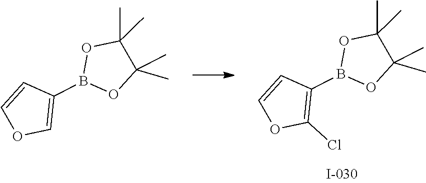

To a stirred solution of 2-(furan-3-yl)-4,4,5,5-tetramethyl-1,3,2-dioxaborolane (500 mg, 2.58 mmol) in DMF (5.0 mL) was added NCS (361.28 mg, 2.71 mmol) portion wise at 0° C. and the resulting mixture was stirred for 4 hours at RT. Upon completion, reaction mixture was diluted with ethyl acetate and washed with water. Combined organic extracts were washed with brine, dried over anhydrous Na₂SO₄, filtered and concentrated under reduced pressure to obtain 400 mg of crude 2-(2-chlorofuran-3-yl)-4,4,5,5-tetramethyl-1,3,2-dioxaborolane (I-030) that was used in subsequent step without further purification. GCMS (EI, m/z)=228.2.

### Synthesis of 2-cyclohexyl-1H-pyrrole (I-031)

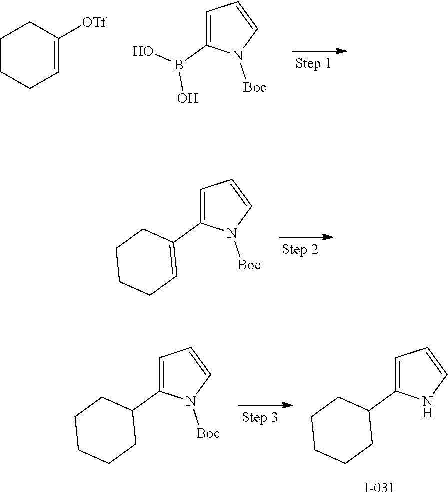 [SMILES: O=S(=O)(OC1=CCCCC1)C(F)(F)F ; CC(C)(C)OC(=O)n1cccc1B(O)O ; CC(C)(C)OC(=O)n1cccc1C1=CCCCC1 ; CC(C)(C)OC(=O)n1cccc1C1CCCCC1 ; c1c[nH]c(C2CCCCC2)c1]

Step 1: To a stirred mixture of (1-(tert-butoxycarbonyl)-1H-pyrrol-2-yl)boronic acid (5.5 g, 26.064 mmol) in THF/Water (10:1, 50 mL) was added Na₂CO₃ (6.90 g, 65.161 mmol) and the mixture was degassed for 15 min with argon. PdC₂(PPh₃)₂ (1.52 g, 2.172 mmol) and cyclohex-1-en-1-yl trifluoromethanesulfonate (5 g, 21.72 mmol) were added and the reaction mixture was heated at 80° C. for 12 hours. The reaction mixture was cooled to RT and filtered through a celite bed. Filtrate was collected, washed with water and aqueous brine solution, dried over anhydrous Na₂SO₄, filtered and concentrated. Crude material was purified by FCC on silica gel using a gradient of EtOAc (0-20%) in Hexane to afford 4 g (74%) of tert-butyl 2-(cyclohex-1-en-1-yl)-1H-pyrrole-1-carboxylate. ¹H NMR (400 MHz, DMSO-d6): δ ppm 7.15-7.14 (m, 1H), 6.10 (t, 1H), 5.98-5.96 (m, 1H), 5.64-5.63 (m, 1H), 2.12-2.07 (m, 4H), 1.69-1.62 (m, 2H), 1.61-1.54 (m, 2H), 1.51 (s, 9H).

Step 2: A stirred mixture of tert-butyl 2-(cyclohex-1-en-1-yl)-1H-pyrrole-1-carboxylate (3.3 g, 13.342 mmol) in EtOAc/EtOH (1:1, 40 mL) was degassed with argon for 5 mins. Then 5 mol % Pd/C (2.5 g) was added and the reaction was stirred under Hydrogen atmosphere for 1 hour at RT. The reaction mixture was filtered through a celite bed and the filter cake was washed with 10% MeOH/DCM several time. The filtrate was evaporated under reduced pressure and the crude thus obtained was purified by FCC on silica gel eluting with Hexane to afford 880 mg (26%) of tert-butyl 2-cyclohexyl-1H-pyrrole-1-carboxylate. ¹H NMR (400 MHz, DMSO-d6): δ ppm 7.15-7.13 (m, 1H), 6.09-6.07 (m, 1H), 5.99-5.98 (m, 1H), 3.08-3.07 (m, 1H), 1.93-1.90 (m, 2H), 1.76-1.73 (m, 2H), 1.70-1.66 (m, 1H), 1.55 (s, 9H), 1.34-1.16 (m, 5H).

Step 3: A mixture of tert-butyl 2-cyclohexyl-1H-pyrrole-1-carboxylate (880.0 mg, 3.529 mmol) and ethylene glycol (20.53 mL) was heated at reflux (180° C.) for 30 min. Reaction mixture was cooled to RT and partitioned between water (20 mL) and dichloromethane (50 mL). Organic layer was separated, dried over anhydrous Na₂SO₄, filtered and evaporated. The residue was purified by FCC on silica gel using a gradient of EtOAc (0-5%) in Hexane to afford 492 mg (93%) of 2-cyclohexyl-1H-pyrrole (I-031). LCMS (ES+, m/z) [M+H]⁺=150.17.

### Synthesis of 2-(tetrahydrofuran-3-yl)-1-tosyl-1H-pyrrole (I-032)

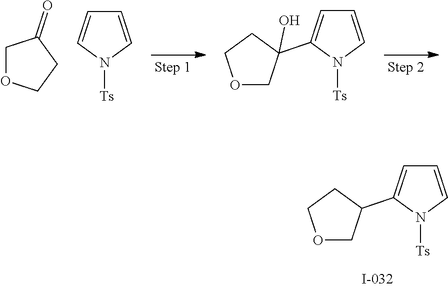

Step 1: A mixture of 1-tosyl-1H-pyrrole (3.0 g, 13.558 mmol) in dry THF (20.0 ml) was cooled to −78° C. and 1.7M tert-Butyllithium (8.8 ml, 14.914 mmol) was added drop wise. After complete addition, reaction mixture was stirred for 2 hours at −78° C. To this mixture, dihydrofuran-3(2H)-one (1.052 mL, 13.558 mmol) in THF (10 mL) was added and the reaction mixture was stirred at RT overnight. The reaction mixture was quenched with saturated aqueous NH₄Cl solution and the aqueous mixture was extracted with ethyl acetate (2×50 mL). The organic phase was washed with brine solution, dried over anhydrous Na₂SO₄, filtered and concentrated under reduced pressure. The residue was purified by FCC over silica gel using a gradient of EtOAc (15 to 20%) in Hexane to afford 800 mg (19%) of 3-(1-tosyl-1H-pyrrol-2-yl) tetrahydrofuran-3-ol. ¹H NMR (400 MHz, DMSO-d6): δ ppm 7.82 (d, 2H), 7.48-7.47 (m, 1H), 7.37 (d, 2H), 6.31-6.29 (m, 1H), 6.22 (t, 1H), 5.14 (s, 1H), 4.06-4.02 (m, 1H), 3.85-3.76 (m, 3H), 2.36 (s, 3H), 2.32 (t, 1H), 2.24-2.18 (m, 1H).

Step 2: To a stirred mixture of 3-(1-tosyl-1H-pyrrol-2-yl) tetrahydrofuran-3-ol (533 mg, 1.734 mmol) in DCE (5 mL) was added Et₃SiH (1.18 ml, 6.936 mmol) and TFA (0.664 ml, 8.671 mmol) at RT and the reaction mixture was irradiated under microwave at 70° C. for 2 hours. After completion, volatiles were removed under reduced pressure. The reaction mixture was diluted with ethyl acetate (40 mL) and washed with saturated aqueous NaHCO₃and brine solution. Organic phase was dried over anhydrous Na₂SO₄, filtered and concentrated under reduced pressure. The residue was purified by FCC over silica gel using a gradient of EtOAc (20 to 30%) in Hexane to afford 450 mg (89%) of 2-(tetrahydrofuran-3-yl)-1-tosyl-1H-pyrrole (I-032). ¹H NMR (400 MHz, DMSO-d6): δ ppm 7.74 (d, 2H), 7.44 (d, 2H), 7.36-7.35 (m, 1H), 6.29-6.27 (m, 1H), 6.22 (br s, 1H), 3.82-3.71 (m, 2H), 3.69-3.64 (m, 2H), 3.41-3.37 (m, 1H), 2.38 (s, 3H), 2.11-2.06 (m, 1H), 1.81-1.77 (m, 1H).

### Synthesis of 2-(3,3-difluorocyclopentyl)-1H-pyrrole (I-034)

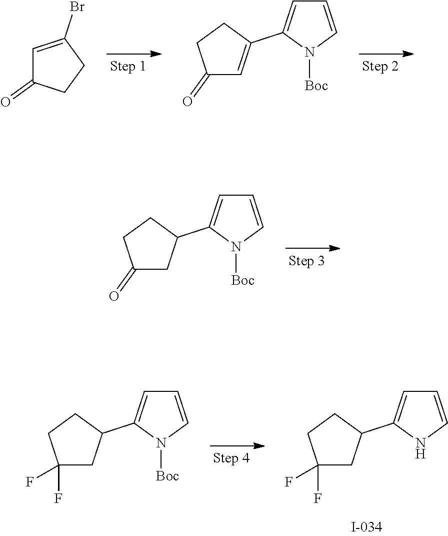 [SMILES: O=C1C=C(Br)CC1 ; CC(C)(C)OC(=O)n1cccc1C1=CC(=O)CC1 ; CC(C)(C)OC(=O)n1cccc1C1CCC(=O)C1 ; CC(C)(C)OC(=O)n1cccc1C1CCC(F)(F)C1 ; FC1(F)CCC(c2ccc[nH]2)C1]

Step 1: To a stirred solution of 3-bromocyclopent-2-en-1-one (3.0 g, 18.756 mmol) and (1-(tert-butoxycarbonyl)-1H-pyrrol-2-yl)boronic acid (5.936 g, 28.134 mmol) in THF/water (3:1, 16.0 mL) was added Na₂CO₃ (3.976 g, 37.512 mmol). Reaction mixture was degassed for 15 minutes under argon, followed by the addition of Pd(OAc)₂ (0.212 g, 0.938 mmol). The reaction mixture was heated at 90° C. for 12 hours. After completion, reaction mixture was filtered through small pad of celite. Filtrate was evaporated and crude thus obtained was purified by FCC over silica gel using a gradient of EtOAc (0 to 15%) in hexane to afford 3.43 g (74%) of tert-butyl 2-(3-oxocyclopent-1-en-1-yl)-1H-pyrrole-1-carboxylate. ¹H NMR (400 MHz, DMSO-d6): δ ppm 7.54 (s, 1H), 6.87-6.86 (m, 1H), 6.37-6.36 (m, 1H), 6.22 (s, 1H), 2.96-2.94 (m, 2H), 2.39-2.37 (m, 2H), 1.54 (s, 9H).

Step 2: In a sealed tube containing tert-butyl 2-(3-oxocyclopent-1-en-1-yl)-1H-pyrrole-1-carboxylate (3 g, 12.14 mmol) in IPA (100.0 mL) were added [IrCp*Cl₂]₂ (97 mg, 0.121 mmol), and K₂CO₃ (84 mg, 0.607 mmol). The reaction mixture was stirred at 85° C. for 5 hours. The solvent was removed under reduced pressure. The residue obtained was purified by FCC over silica gel using a gradient of EtOAc (5 to 10%) in Hexane to afford 1.45 g (48%) of tert-butyl 2-(3-oxocyclopentyl)-1H-pyrrole-1-carboxylate. ¹H NMR (400 MHz, DMSO-d6): δ ppm 7.21-7.20 (m, 1H), 6.13-6.10 (m, 2H), 3.88-3.85 (m, 1H), 2.59-2.50 (m, 1H), 2.34-2.30 (m, 1H), 2.23-2.17 (m, 3H), 1.91-1.84 (m, 1H), 1.55 (s, 9H).

Step 3: To a well degassed mixture of tert-butyl 2-(3-oxocyclopentyl)-1H-pyrrole-1-carboxylate (680 mg, 2.728 mmol) in dry DCM (10.0 mL) was added Bis(2-methoxyethyl)aminosulfur trifluoride (50% in toluene, 3.016 mL, 6.819 mmol) drop wise and the reaction mixture was stirred at RT for 24 hours. The reaction mixture was diluted with DCM (30.0 mL) and poured into ice cold saturated sodium bicarbonate solution. Organic phase was separated, dried over anhydrous Na₂SO₄, filtered and concentrated under reduced pressure. Crude thus obtained was purified by FCC over silica gel using a gradient of EtOAc (0 to 10%) in hexane to afford 190 mg (26%) of tert-butyl 2-(3,3-difluorocyclopentyl)-1H-pyrrole-1-carboxylate. ¹H NMR (400 MHz, DMSO-d6): δ ppm 7.19 (s, 1H), 6.16-6.11 (m, 2H), 3.79-3.76 (m, 1H), 2.32-2.02 (m, 5H), 1.82-1.71 (m, 1H), 1.55 (s, 9H).

Step 4: A mixture of tert-butyl 2-(3,3-difluorocyclopentyl)-1H-pyrrole-1-carboxylate (230.0 mg, 0.848 mmol) and ethylene glycol (5.0 mL) was heated at 180° C. for 30 minutes. After completion, reaction mixture was cooled and partitioned between water and dichloromethane. The organic phase was dried over anhydrous Na₂SO₄, filtered and evaporated under reduced pressure. The residue was purified by FCC over silica gel using a gradient of EtOAc (0 to 10%) in hexane to afford 136 mg (94%) of 2-(3,3-difluorocyclopentyl)-1H-pyrrole (I-034). LCMS (ES+, m/z) [M+H]+=172.2. ¹H NMR (400 MHz, DMSO-d6): δ ppm 10.61 (s, 1H), 6.61-6.60 (m, 1H), 5.89-5.88 (m, 1H), 5.80 (s, 1H), 3.29-3.24 (m, 1H), 2.46-2.40 (m, 1H), 2.26-2.09 (m, 4H), 1.81-1.76 (m, 1H).

### SYNTHESIS OF EXAMPLES

### Synthesis of N-(4-cyano-2-fluorophenyl)-5-(2-fluorophenyl)-1H-pyrrole-3-sulfonamide (Cpd 014)

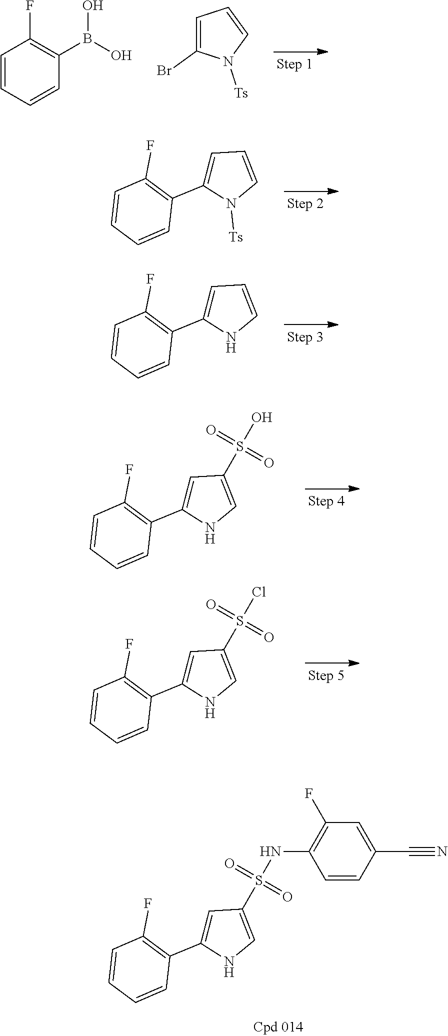

Step 1: To a solution of 2-bromo-1-tosyl-1H-pyrrole (4 g, 13 mmol) and (2-fluorophenyl)boronic acid (3 g, 27 mmol) in toluene (40 mL) and water (1 mL) was added Na₂CO₃ (2.1 g, 20 mmol). The RM was degassed before the addition of Pd(PPh₃)₄ (0.15 g, 0.13 mmol) at RT under N₂ atmosphere. The RM was stirred for 8 h at 100° C. until completion. After cooling to RT, the volatiles were removed under reduced pressure. The residue was purified by FCC on silica gel using a gradient of EtOAc (0 to 10%) in hexane to afford 3.5 g (83%) of 2-(2-fluorophenyl)-1-tosyl-1H-pyrrole. ¹H NMR (400 MHz, CDCl₃): δ ppm 7.45-7.44 (m, 1H), 7.38-7.36 (m, 1H), 7.28 (d, 2H), 7.17-7.07 (m, 4H), 7.03-6.98 (m, 1H), 6.34-6.32 (t, 1H), 6.22 (bs, 1H), 2.36 (s, 3H).

Step 2: To a solution of 2-(2-fluorophenyl)-1-tosyl-1H-pyrrole (3.5 g, 11 mmol) in a mixture of MeOH/Water (5/1) (60 mL), was added NaOH (2.2 g, 55 mmol) portion wise at 0° C. The RM was stirred 60° C. for 16 h. After cooling to RT, the volatiles were removed under reduced pressure.

The residue was dissolved in DCM and washed with water and brine. The organic layer was dried over Na₂SO₄ and filtrated. The volatiles were removed under reduced pressure to afford 1.5 g (83%) of 2-(2-fluorophenyl)-1H-pyrrole. ¹H NMR (400 MHz, CDCl₃): δ ppm 9.02 (bs, 1H), 7.63-7.59 (m, 1H), 7.17-7.06 (m, 3H), 6.9 (bs, 1H), 6.65 (bs, 1H), 6.31 (bs, 1H).

Step 3: To a solution of 2-(2-fluorophenyl)-1H-pyrrole (1.5 g, 9.3 mmol) in MeCN (30 ml) was added Py·SO₃ (2.22 g, 13.96 mmol) at RT. The RM was stirred at 120° C. for 3 h. The RM was concentrated under reduced pressure. The residue was dissolved in water and washed with DCM. The aqueous phase was concentrated under reduced pressure to afford 4 g of 5-(2-fluorophenyl)-1H-pyrrole-3-sulfonic acid, which was used without further purification.

Step 4: To a solution of 5-(2-fluorophenyl)-1H-pyrrole-3-sulfonic acid (3.0 g, 12 mmol) in MeCN (35 mL) was added POCl₃ (1.2 mL, 12 mmol) at 0° C. The RM was stirred at 70° C. for 3 h. The RM was poured onto the ice water. Aqueous part was extracted twice with DCM. Combined organic layer was washed with water, brine and dried over Na₂SO₄ to afford 1.7 g of 5-(2-fluorophenyl)-1H-pyrrole-3-sulfonyl chloride, which was used without further purification.

Step 5: To a solution of 5-(2-fluorophenyl)-1H-pyrrole-3-sulfonyl chloride (0.3 g, 1.2 mmol) in pyridine (5 ml) was added 4-(trifluoromethyl)aniline (0.3 g, 1.7) at 0° C. The RM was stirred at 80° C. for 16 h. The RM was concentrated, diluted with water, and extracted in DCM. The organic layers were combined, washed with brine, dried over Na₂SO₄, and concentrated under reduced pressure. The residue was purified by FCC over silica using a gradient of EtOAc (0-60%) in hexane to afford 0.05 g (20%) of N-(4-cyano-2-fluorophenyl)-5-(2-fluorophenyl)-1H-pyrrole-3-sulfonamide (Cpd 014).

The following compounds were prepared in a similar manner (use of appropriate starting material, intermediates, reagents and purification methods (including chiral HPLC or chiral SFC) known to the person skilled in the art or as described herein) as described for Cpd 014: Cpd 002; Cpd 003; Cpd 004; Cpd 005; Cpd 006; Cpd 007; Cpd 008; Cpd 010; Cpd 011; Cpd 012; Cpd 015; Cpd 016; Cpd 017; Cpd 021; Cpd 022; Cpd 024; Cpd 025; Cpd 027; Cpd 028; Cpd 029; Cpd 030; Cpd 031; Cpd 032; Cpd 035; Cpd 036; Cpd 037; Cpd 115; Cpd 116; Cpd 126; Cpd 127; Cpd 128; Cpd 129; Cpd 130; Cpd 133; Cpd 134; Cpd 135; Cpd 136; Cpd 137; Cpd 138; Cpd 139; Cpd 140; Cpd 141; Cpd 142; Cpd 143; Cpd 144; Cpd 145; Cpd 146; Cpd 147; Cpd 164; Cpd 167; Cpd 168; Cpd 175; Cpd 176; Cpd 187; Cpd 199; Cpd 312; Cpd 313; Cpd 317; Cpd 326; Cpd 333; Cpd 334; Cpd 338; Cpd 375; Cpd 422; Cpd 435; Cpd 436; Cpd 464; Cpd 576 (from I-020) and Cpd 579.

### Synthesis of 5-(2-fluorophenyl)-N-[2-fluoro-4-(trifluoromethyl)phenyl]-1H-pyrrole-3-sulfonamide (Cpd 055)

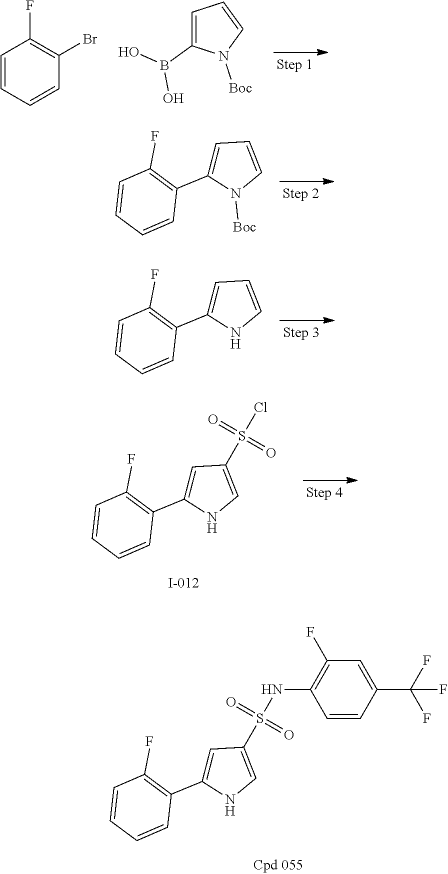

Step 1: To a mixture of 1-(tert-butoxycarbonyl)pyrrol-2-ylboronic acid (15 g, 71 mmol) and 1-bromo-2-fluorobenzene (18.7 g, 106.6 mmol) in a mixture of dioxane (120 mL) and H₂O (6 mL) were added CsF (32.4 g, 213 mmol) and Pd(dppf)Cl₂ (2.60 g, 3.55 mmol) at RT. The RM was stirred for 5 h at 100° C. under N₂. After completion, the RM was concentrated under reduced pressure. The residue was purified by FCC over silica using as eluent PE/EtOAc (3/1) to afford 17 g (92%) of tert-butyl 2-(2-fluorophenyl)pyrrole-1-carboxylate. ¹H NMR (300 MHz, DMSO-d6) δ ppm 7.44-7.35 (m, 3H), 7.26-7.19 (m, 2H), 6.36-6.28 (m, 2H), 1.30 (s, 9H).

Step 2: To a solution of tert-butyl 2-(2-fluorophenyl)pyrrole-1-carboxylate (17 g, 65 mmol,) in MeOH (60 mL) was added MeONa (58.6 g, 325 mmol, 30% wt in MeOH) dropwise at RT. The RM was stirred for 3 h at 50° C. The RM was concentrated under reduced pressure. The residue was dissolved in EtOAc (600 mL), washed with water (300 mL), and brine (300 mL), dried over Na₂SO₄, filtered, and concentrated under reduced pressure. The residue was purified by FCC over silica using as eluent PE/EtOAc (4/1) to afford 10 g (97%) of 2-(2-fluorophenyl)-1H-pyrrole.

Step 3: To a mixture of 2-(2-fluorophenyl)-1H-pyrrole (10 g, 62 mmol) in MeCN (160 mL) was added Py·SO₃ (10.4 g, 65 mmol) at RT under N₂ atmosphere. The RM was stirred for 3 h at 120° C. under N₂ atmosphere. After cooling at RT, POCl₃ (47.7 g, 311 mmol) was added dropwise to the RM. The RM was stirred 3 h at 70° C. under N₂ atmosphere. The RM was concentrated under reduced pressure. The residue was poured into ice-water, and then extracted with EtOAc (3×200 mL). The organic layers were combined, dried over Na₂SO₄, filtrated, and concentrated under reduced pressure to afford 12 g of 5-(2-fluorophenyl)-1H-pyrrole-3-sulfonyl chloride (I-012), which was used without further purification.

Step 4: To a solution of NaH (60% in mineral oil) (308 mg, 7.70 mmol) and 2-fluoro-4-(trifluoromethyl)aniline (690 mg, 3.85 mmol) in THF (10 mL) was added a solution of 5-(2-fluorophenyl)-1H-pyrrole-3-sulfonyl chloride (I-012) (500 mg) in THF (3 mL) at 0° C. under N₂ atmosphere. The RM was stirred for 3 h at RT. The reaction was quenched by addition of ice-water (1 ml). The RM was extracted with EtOAc (100 ml), washed with water (100 mL) and brine (100 mL), dried over Na₂SO₄, and concentrated under reduced pressure. The residue was purified by R^{p} flash chromatography on C18 gel using a gradient of MeCN (40 to 60%) in water with 0.1% FA to afford 190 mg (25%) of N-[2-fluoro-4-(trifluoromethyl)phenyl]-5-(2-fluorophenyl)-1H-pyrrole-3-sulfonamide (Cpd 055).

The following compounds were prepared in a similar manner (use of appropriate reagents and purification methods (including chiral HPLC or chiral SFC) known to the person skilled in the art) as described for Cpd 055: Cpd 038; Cpd 039; Cpd 040; Cpd 041; Cpd 042; Cpd 043; Cpd 044; Cpd 045; Cpd 046; Cpd 047; Cpd 048; Cpd 049; Cpd 050; Cpd 051; Cpd 052; Cpd 053; Cpd 054; Cpd 056; Cpd 057; Cpd 058; Cpd 059; Cpd 062; Cpd 064; Cpd 065 (from I-017); Cpd 066; Cpd 068; Cpd 069; Cpd 070; Cpd 072; Cpd 073; Cpd 074; Cpd 075; Cpd 155; Cpd 156; Cpd 170; Cpd 180; Cpd 191; Cpd 214 and Cpd 248.

### Synthesis of 5-(2-fluorophenyl)-N-[3-methoxy-5-(trifluoromethyl)pyridin-2-yl]-1H-pyrrole-3-sulfonamide (Cpd 060) from I-012

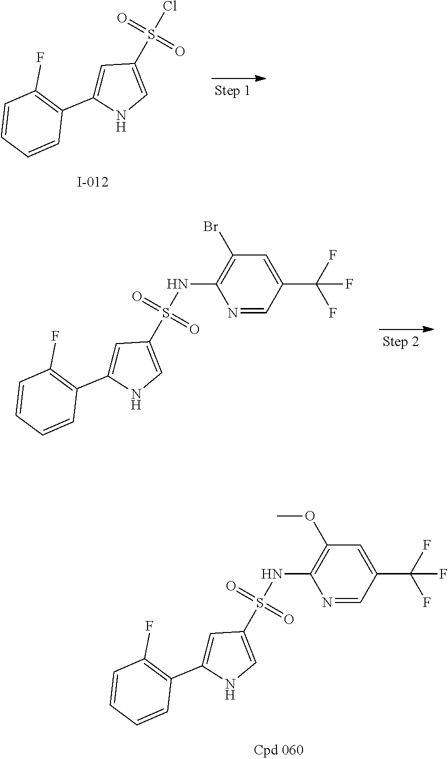 [SMILES: O=S(=O)(Cl)c1c[nH]c(-c2ccccc2F)c1 ; O=S(=O)(Nc1ncc(C(F)(F)F)cc1Br)c1c[nH]c(-c2ccccc2F)c1 ; COc1cc(C(F)(F)F)cnc1NS(=O)(=O)c1c[nH]c(-c2ccccc2F)c1]

Step 1: To a solution of NaH (60% in mineral oil) (770 mg, 19.3 mmol) and 3-bromo-5-(trifluoromethyl)pyridin-2-amine (1.86 g, 7.72 mmol) in THF (20 mL) was added 5-(2-fluorophenyl)-1H-pyrrole-3-sulfonyl chloride (I-012) (1.00 g) in THF (5 mL) dropwise at 0° C. The RM was stirred for 3 h at RT. The reaction was quenched by ice-water. The mixture was dissolved in EtOAc (100 ml). The organic layer was washed with water (50 mL) and brine (50 mL), dried over Na₂SO₄, filtrated and concentrated. The residue was purified by R^{p} flash chromatography on C18 gel using a gradient of MeCN (40 to 60%) in water with 0.1% FA to afford 300 mg (40%) of N-[3-bromo-5-(trifluoromethyl)pyridin-2-yl]-5-(2-fluorophenyl)-1H-pyrrole-3-sulfonamide.

Step 2: To a mixture of N-[3-bromo-5-(trifluoromethyl) pyridin-2-yl]-5-(2-fluorophenyl)-1H-pyrrole-3-sulfonamide (200 mg, 0.43 mmol) and CuBr (25 mg, 0.17 mmol) in MeOH (5 mL) were added MeONa (0.8 mL, 4.31 mmol, 5M in MeOH) and EtOAc (23 mg, 0.26 mmol) at RT. The RM was stirred for 4 h at 100° C. under nitrogen atmosphere. The volatiles were removed under reduced pressure. The residue was dissolved in DCM (50 mL). The organic layer was washed with water (30 mL) and with brine (30 mL), dried over Na₂SO₄, filtrated, and concentrated under reduced pressure. The residue was purified by FCC over silica gel using a gradient of EtOAc (0 to 30%) in PE. The residue was purified again by preparative HPLC on a XBridge Prep C18 OBD Column (19×150 mm, 5 μm); Mobile Phase A: Water (10 mM NH₄HCO₃), Mobile Phase B: MeCN; Flow rate: 25 mL/min; Gradient: 38% to 40% of B in 8 min. The purification afforded 74 mg (41%) of 5-(2-fluorophenyl)-N-[3-methoxy-5-(trifluoromethyl)pyridin-2-yl]-1H-pyrrole-3-sulfonamide (Cpd 060).

### Synthesis of 5-(5-chloro-2-fluorophenyl)-N-[4-(difluoromethoxy)-2,5-difluorophenyl]-1H-pyrrole-3-sulfonamide (Cpd 410)

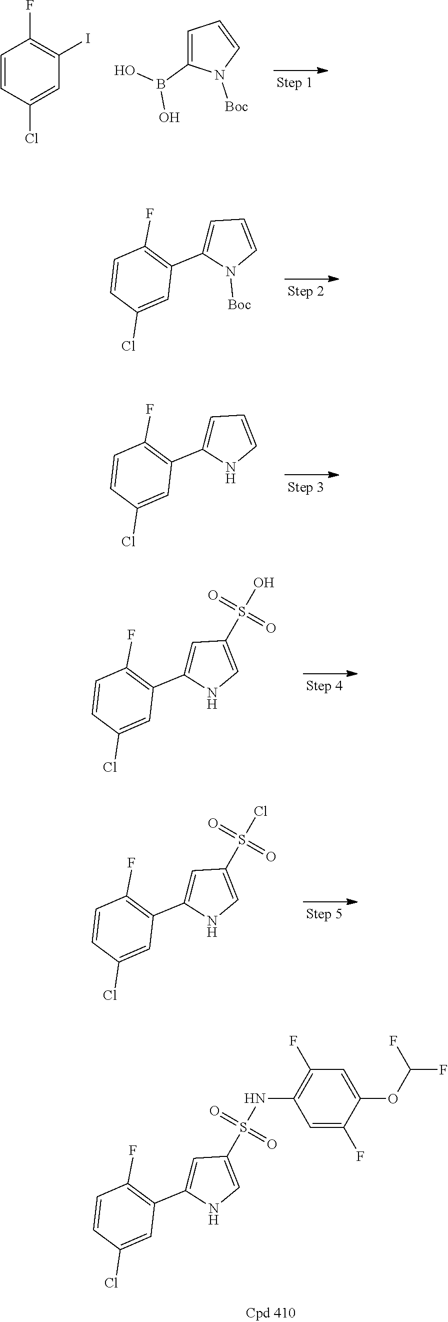

Step 1: To a stirred mixture of 4-chloro-1-fluoro-2-iodobenzene (5 g, 19.5 mmol) and 1-(tert-butoxycarbonyl) pyrrol-2-ylboronic acid (4.11 g, 19.5 mmol) in THF (100 mL) and water (10 mL) were added K₂CO₃ (8.08 g, 58.5 mmol) and Pd(PPh₃)₄ (2.25 g, 1.95 mmol) in one portion at RT under nitrogen atmosphere. The resulting mixture was stirred for 18 h at 100° C. under nitrogen atmosphere. The mixture was allowed to cool down to RT. The resulting mixture was concentrated under vacuum. The residue was purified by FCC on silica gel using a gradient of EtOAc (0-10%) in petroleum ether to afford 5.2 g (90%) of tert-butyl 2-(5-chloro-2-fluorophenyl) pyrrole-1-carboxylate. ¹H NMR (300 MHz, CHCl₃) δ 7.44 (dd, J=3.2, 1.9 Hz, 1H), 7.36 (dd, J=6.3, 2.7 Hz, 1H), 7.32-7.26 (m, 1H), 7.03 (m, 1H), 6.34-6.25 (m, 2H), 1.43 (s, 9H).

Step 2: To a stirred mixture of tert-butyl 2-(5-chloro-2-fluorophenyl) pyrrole-1-carboxylate (5.2 g, 17.58 mmol) and MeONa (4.75 g, 87.9 mmol) in MeOH (80 mL) was stirred for 16 h at 60° C. under nitrogen atmosphere. The resulting mixture was concentrated under reduced pressure. The aqueous layer was extracted with EtOAc (2×300 mL), dried over anhydrous Na₂SO₄. After filtration, the filtrate was concentrated under reduced pressure. The residue was purified by FCC on silica gel using a gradient of EtOAc (0-10%) in petroleum ether to afford 3.1 g (90%) of 2-(5-chloro-2-fluorophenyl)-1H-pyrrole (3.1 g, 90%). ¹H NMR (300 MHz, CHCl₃) δ 9.01 (s, 1H), 7.62 (dd, J=6.9, 2.4 Hz, 1H), 7.18-7.01 (m, 2H), 6.96 (m, 1H), 6.72 (m, 1H), 6.39 (m, 1H).

Step 3: To a stirred solution of 2-(5-chloro-2-fluorophenyl)-1H-pyrrole (3.1 g, 15.85 mmol) in pyridine (160 mL) was added Py·SO₃ (2.52 g, 15.85 mmol) at RT under argon atmosphere. The resulting mixture was stirred for 3 h at 100° C. under argon atmosphere. The mixture was allowed to cool down to RT. The reaction was concentrated under reduced pressure and extracted with CHCl₃ (3×300 mL). The aqueous phase was concentrated under reduced pressure to afford 3.8 g (87%) of crude 5-(5-chloro-2-fluorophenyl)-1H-pyrrole-3-sulfonic acid (3.8 g, 87%) that was used for subsequent step without further purification.

Step 4: To a stirred solution of 5-(5-chloro-2-fluorophenyl)-1H-pyrrole-3-sulfonic acid (3.8 g, 13.78 mmol) in MeCN (30 mL) was added POCl₃ (2.54 g, 16.54 mmol) dropwise at RT under argon atmosphere. The resulting mixture was stirred for 3 h at 70° C. under argon atmosphere. The mixture was allowed to cool down to RT. The reaction was quenched with water at RT. The resulting mixture was extracted with DCM (3×300 mL). The combined organic layers were washed with brine (3×100 mL), dried over anhydrous Na₂SO₄, filtered and concentrated under reduced pressure to afford 1.8 g (44%) of crude 5-(5-chloro-2-fluorophenyl)-1H-pyrrole-3-sulfonyl chloride that was used in subsequent steps without further purification.

Step 5: A mixture of 5-(5-chloro-2-fluorophenyl)-1H-pyrrole-3-sulfonyl chloride (600 mg, 2.04 mmol) and 4-(difluoromethoxy)-2,5-difluoroaniline (597 mg, 3.06 mmol) in pyridine (10 mL) was stirred for 12 h at 80° C. under nitrogen atmosphere. The mixture was allowed to cool down to RT. The resulting mixture was concentrated under vacuum. The residue was purified by reverse FCC on silica gel using a gradient of MeCN (30-50%) in water to afford 200 mg (22%) of 5-(5-chloro-2-fluorophenyl)-N-[4-(difluoromethoxy)-2,5-difluorophenyl]-1H-pyrrole-3-sulfonamide (Cpd 410).

The following compounds were prepared in a similar manner (use of appropriate reagents and purification methods (including chiral HPLC or chiral SFC) known to the person skilled in the art) as described for Cpd 410: Cpd 409, Cpd 411, Cpd 412, Cpd 413, Cpd 414, Cpd 446; Cpd 447; Cpd 448 (from I-017); Cpd 449 (from I-017); Cpd 450 (from I-017); Cpd 451 from (I-017); Cpd 508; Cpd 509; Cpd 510; Cpd 511; Cpd 512; Cpd 513; Cpd 514; Cpd 522; Cpd 523; Cpd 524; Cpd 525; Cpd 526; Cpd 527; Cpd 529 and Cpd 533.

### Synthesis of 5-(5-chloro-2,4-difluorophenyl)-N-(5-chloro-4-cyano-2-fluorophenyl)-1H-pyrrole-3-sulfonamide (Cpd 470)

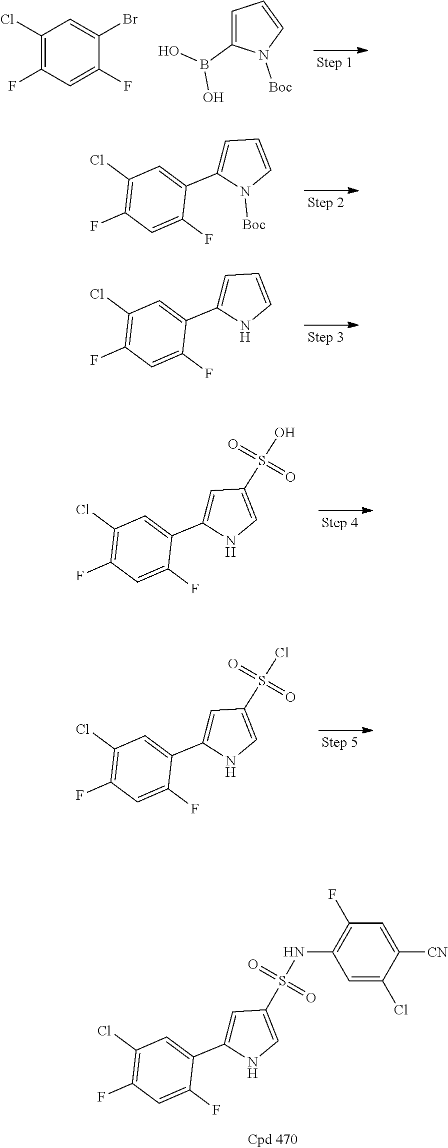

Step 1: To a stirred mixture of (1-(tert-butoxycarbonyl)-1H-pyrrol-2-yl)boronic acid (1 g, 4.739 mmol) and 1-bromo-5-chloro-2,4-difluorobenzene (1.078 g, 4.739 mmol) in THF/water (3:1, 40.0 mL) was added K₂CO₃ (1.308 g, 9.478 mmol) and the reaction mixture was degassed with argon for 15 minutes. Then Pd(PPh₃)₄ (274 mg, 0.237 mmol) was added to the reaction mixture and the reaction mixture was heated at 80° C. for 16 hours. Reaction mixture was partitioned between EtOAc and water. Organic layer was separated, dried over anhydrous Na₂SO₄, filtered and evaporated under reduced pressure to get crude material. Crude thus obtained was purified by FCC on silica gel using a gradient of EtOAc (0%-5%) in hexane to afford 1.4 g (94%) of tert-butyl 2-(5-chloro-2,4-difluorophenyl)-1H-pyrrole-1-carboxylate. ¹H NMR (400 MHz, DMSO-d6): δ ppm 7.69 (t, 1H), 7.62 (t, 1H), 7.43-7.41 (m, 1H), 6.40-6.39 (m, 1H), 6.33 (t, 1H), 1.34 (s, 9H).

Step 2: To a stirred mixture of tert-butyl 2-(5-chloro-2,4-difluorophenyl)-1H-pyrrole-1-carboxylate (1.4 g, 4.462 mmol) in dry MeOH (20.0 mL) was added MeONa (25% in MeOH, 2.4 g, 44.621 mmol) and the reaction mixture was heated at 80° C. for 16 hours. Reaction mixture was evaporated and partitioned between EtOAc and water. Organic layer was separated, dried over anhydrous Na₂SO₄, filtered and evaporated under reduced pressure to get crude material. Crude thus obtained was purified by FCC on silica gel using a gradient of EtOAc (0%-2%) in hexane to afford 600 mg (63%) of 2-(5-chloro-2,4-difluorophenyl)-1H-pyrrole. LCMS (ES−, m/z) [M−H]⁻=212.07. ¹H NMR (400 MHz, DMSO-d6): δ ppm 11.34 (s, 1H), 7.95 (t, 1H), 7.58-7.53 (m, 1H), 6.94 (s, 1H), 6.57 (s, 1H), 6.18-6.17 (m, 1H).

Step 3: Py·SO₃ complex (447.05 mg, 2.809 mmol) was added to a stirred solution of 2-(5-chloro-2,4-difluorophenyl)-1H-pyrrole (600 mg, 2.809 mmol) in dry MeCN (15.0 mL). Reaction mixture was then heated at 80° C. for 16 hours. After completion, reaction mixture was evaporated under reduced pressure and partitioned between DCM and water. Aqueous phase was lyophilized to afford 670 mg of crude 2-(5-chloro-2,4-difluorophenyl)-1H-pyrrole that was used in subsequent step without further purification. LCMS (ES−, m/z) [M−H]⁻=292.03.

Step 4: POCl₃ (0.32 mL, 3.422 mmol) was added to a stirred mixture of 2-(5-chloro-2,4-difluorophenyl)-1H-pyrrole (670 mg) in dry MeCN (15.0 mL). Reaction mixture was then heated at 70° C. for 16 hours. Reaction mixture was evaporated and crude residue was partitioned between DCM and water. Organic layer was separated, washed with brine solution, dried over anhydrous Na₂SO₄, filtered and evaporated under reduced pressure to get 370 mg of crude 5-(5-chloro-2,4-difluorophenyl)-1H-pyrrole-3-sulfonyl chloride that was used in subsequent step without further purification. LCMS (ES+, m/z) [M+H]⁺=376.10 (quenched with N-methyl piperazine).

Step 5: To a stirred mixture of 5-(5-chloro-2,4-difluorophenyl)-1H-pyrrole-3-sulfonyl chloride (185 mg, 0.593 mmol) and 4-amino-2-chloro-5-fluorobenzonitrile (101 mg, 0.593 mmol) in Pyridine (2.5 mL) was added DMAP (14.48 mg, 0.119 mmol). Reaction mixture was then heated at 100° C. for 16 hours. After completion, all the volatiles were removed under reduced pressure. The residue was purified by R^{p} preparative HPLC on a YMC-Actus Triart C18 column (20×250 mm, 5 μm) operating with a flow rate of 16 mL/min; Mobile Phase A: 20 mM NH₄HCO₃ in water; Mobile Phase B: MeCN; Gradient profile: 30% B for 3 min, then 30% B to 65% in 18 min and to 95% in 1 minute, held for 2 min for column washing, then returned to initial composition in 1 min and held for 2 min. The purification afforded 95 mg (36%) of 5-(5-chloro-2,4-difluorophenyl)-N-(5-chloro-4-cyano-2-fluorophenyl)-1H-pyrrole-3-sulfonamide (Cpd 470).

The following compounds were prepared in a similar manner (use of appropriate reagents and purification methods (including chiral HPLC or chiral SFC) known to the person skilled in the art) as described for Cpd 470: Cpd 471, Cpd 492; Cpd 495; Cpd 544; Cpd 587; Cpd 588; Cpd 589; Cpd 597; Cpd 611 (from I-026); Cpd 613 (from I-027); Cpd 655; Cpd 662 and Cpd 665.

### Synthesis of 5-(6-chloro-2-pyridyl)-N-[2,5-difluoro-4-(trifluoromethyl)phenyl]-1H-pyrrole-3-sulfonamide (Cpd 215)

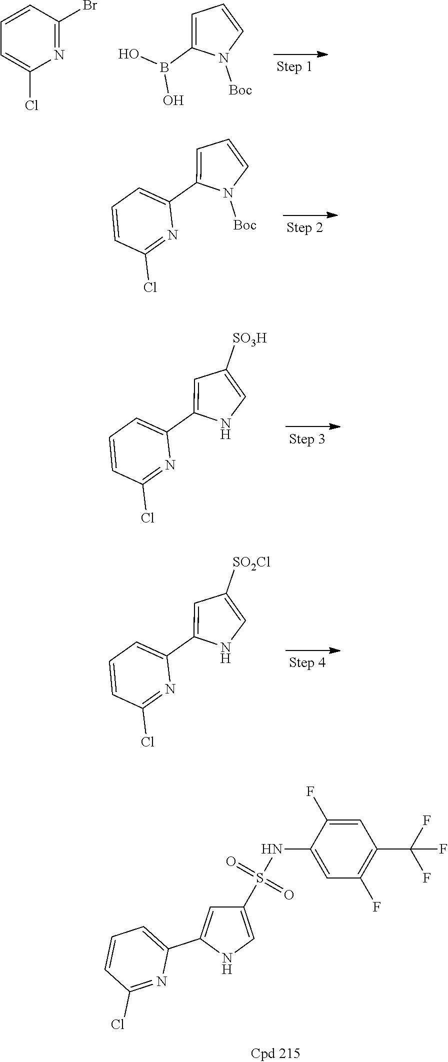 [SMILES: Clc1cccc(Br)n1 ; CC(C)(C)OC(=O)n1cccc1B(O)O ; CC(C)(C)OC(=O)n1cccc1-c1cccc(Cl)n1 ; O=S(=O)(O)c1c[nH]c(-c2cccc(Cl)n2)c1 ; O=S(=O)(Cl)c1c[nH]c(-c2cccc(Cl)n2)c1 ; O=S(=O)(Nc1cc(F)c(C(F)(F)F)cc1F)c1c[nH]c(-c2cccc(Cl)n2)c1]

Step 1: THF (30 mL) and water (12 mL) were added to (1-(tert-butoxycarbonyl)-1H-pyrrol-2-yl)boronic acid (2.19 g, 10 mmol), 2-bromo-6-chloropyridine (4.0 g, 21 mmol) and K₂CO₃ (5.7 g, 41 mmol). The RM was degassed with argon. Pd(PPh₃)₄ (1.2 g, 1.0 mmol) was added. The RM was heated at 60° C. for 2 h. After completion, the RM was filtered over celite bed and extracted with EtOAc. The organic layers were combined, washed with brine, dried over Na₂SO₄, filtrated, and concentrated under reduced pressure. The residue was purified by FCC over silica using a gradient of DCM (0 to 40%) in hexane to afford 1.7 g (29%) of tert-butyl 2-(6-chloropyridin-2-yl)-1H-pyrrole-1-carboxylate. ¹H NMR (400 MHz, DMSO-d6): δ ppm 7.62 (t, 1H), 7.37-7.35 (m, 1H), 7.31 (d, 1H), 7.21 (d, 1H), 1.37 (s, 9H).

Step 2: To tert-butyl 2-(6-chloropyridin-2-yl)-1H-pyrrole-1-carboxylate (1 g, 3.6 mmol) in dry MeCN (20 mL) was added chlorosulfonic acid (1.2 mL, 18 mmol) at 0° C. under N₂ atmosphere. The RM was heated at 70° C. for 1 h. After completion, the RM was poured into ice water and extracted in EtOAc thrice. The organic layers were combined, washed with brine, dried over Na₂SO₄, filtrated, and concentrated under reduced pressure to afford 0.9 g (97%) of 5-(6-chloropyridin-2-yl)-1H-pyrrole-3-sulfonic acid, which was used without further purification.

Step 3: To a solution of 5-(6-chloropyridin-2-yl)-1H-pyrrole-3-sulfonic acid (900 mg, 3.6 mmol) in DCM (10 mL) and was added oxalyl chloride (1.5 mL, 17 mmol) and DMF (2 drops) at 0° C. The RM was stirred for 2 h at 40° C. After completion, the RM was concentrated under reduced pressure, diluted with water, and extracted in EtOAc. The organic layers were combined, washed with brine, dried over Na₂SO₄, filtrated, and concentrated under reduced pressure to afford 450 mg (47%) of 5-(6-chloropyridin-2-yl)-1H-pyrrole-3-sulfonyl chloride, which was used without further purification.

Step 4: To, 2,5-difluoro-4-(trifluoromethyl)aniline (322 mg, 1.6 mmol) in dry MeCN (5 mL) were added 5-(6-chloropyridin-2-yl)-1H-pyrrole-3-sulfonyl chloride (450 mg 1.6 mmol) and pyridine (0.36 mL, 4.1 mmol). The RM was heated under N₂ atmosphere at 70° C. for 16 h. After completion, the RM was concentrated under reduced pressure. The residue was purified by preparative H PLC. The purification was done on Waters auto purification instrument with a YMC Actus Triart C18 (250×20 mm, 5p) column, operating at RT and flow rate of 16 mL/min. Mobile phase A=20 mM NH₄HCO₃ in water, mobile phase B=MeCN. Gradient Profile: Mobile phase initial composition of 80% A and 20% B, then 65% A and 35% B in 2 min, then to 20% A and 80% B in 22 min., then to 5% A and 95% B in 23 min., held this composition up to 25 min. The purification afforded 25 mg (3.5%) of 5-(6-chloropyridin-2-yl)-N-(2,5-difluoro-4-(trifluoromethyl) phenyl)-1H-pyrrole-3-sulfonamide (Cpd 215).

The following compounds were prepared in a similar manner (use of appropriate reagents and purification methods (including chiral HPLC or chiral SFC) known to the person skilled in the art) as described for Cpd 215: Cpd 232; Cpd 233; Cpd 378; Cpd 383 (from I-013); Cpd 399; Cpd 408 (from I-019); Cpd 429; Cpd 434; Cpd 455 (from I-014); Cpd 459 (from I-017); Cpd 463; Cpd 467 (from I-015); Cpd 472 (from I-016); Cpd 494; Cpd 583; Cpd 591 and Cpd 630.

### Synthesis of N-(2,5-difluoro-4-(trifluoromethyl)phenyl)-5-(3-fluoropyridin-2-yl)-1H-pyrrole-3-sulfonamide (Cpd 148)

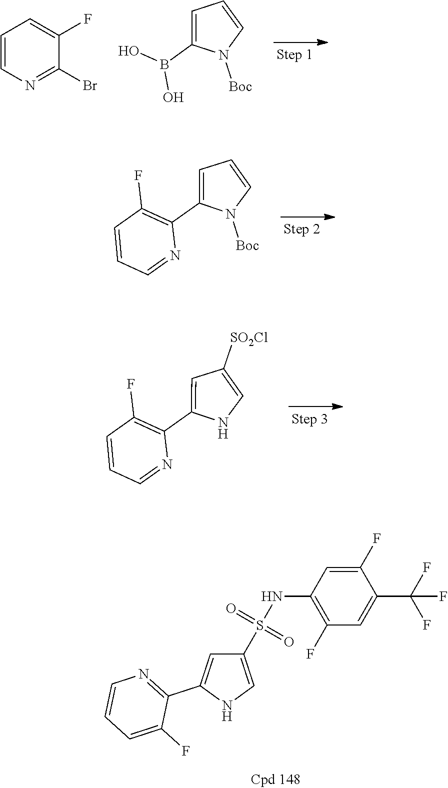 [SMILES: Fc1cccnc1Br ; CC(C)(C)OC(=O)n1cccc1B(O)O ; CC(C)(C)OC(=O)n1cccc1-c1ncccc1F ; O=S(=O)(Cl)c1c[nH]c(-c2ncccc2F)c1 ; O=S(=O)(Nc1cc(F)c(C(F)(F)F)cc1F)c1c[nH]c(-c2ncccc2F)c1]

Step 1: To a solution of (1-(tert-butoxycarbonyl)-1H-pyrrol-2-yl)boronic acid (2.5 g, 12 mmol) and 2-bromo-3-fluoropyridine (1.3 g, 12 mmol) in THF (75 mL) was added an aq. solution of K₂CO₃ (23.6 mL, 1M). The RM was degassed with Ar for 20 min and then Pd(PPh₃)₄ (1.4 g, 1.18 mmol) was added. The RM was heated at 90° C. for 16 h. After cooling to RT, the RM was filtered through celite bed. The filtrate was extracted with EtOAc. The organic layer was washed with water, brine and dried over Na₂SO₄, filtrated, and concentrated under reduced pressure. The residue was purified by FCC on silica gel using a gradient of EtOAc (0-30%) in hexane to afford 3.1 g (58%) of tert-butyl 2-(3-fluoropyridin-2-yl)-1H-pyrrole-1-carboxylate. ¹H NMR (400 MHz, CDCl₃): δ ppm 8.42 (d, 1H), 7.41-7.37 (m, 2H), 7.27-7.25 (m, 1H), 6.51-6.50 (m, 1H), 6.30 (t, 1H), 1.36 (s, 9H).

Step 2: To a solution of tert-butyl 2-(3-fluoropyridin-2-yl)-1H-pyrrole-1-carboxylate (1 g, 3.2 mmol) in MeCN (5 mL), was added Chlorosulfonic acid (1.3 mL, 19 mmol). The RM was heated at 80° C. for 16 h. The volatiles were removed under reduced pressure. The residue was diluted with saturated aq. NaHCO₃solution, extracted with EtOAc (3×20 mL). The combined organic layers were washed with brine and concentrated under reduced pressure to obtain 1 g (65%) of 5-(3-fluoropyridin-2-yl)-1H-pyrrole-3-sulfonyl chloride.

Step 3: To a solution of 5-(3-fluoropyridin-2-yl)-1H-pyrrole-3-sulfonyl chloride (250 mg, 0.96 mmol) and 2,5-difluoro-4-(trifluoromethyl)aniline (345 mg, 1.9 mmol) in MeCN (5 mL) was added pyridine (3.2 mL) and heated at 70° C. for 16 h until completion. The volatiles were removed under reduced pressure. The residue was purified by FCC on silica gel using a gradient elution of EtOAc (0-30%) in hexane to afford 230 mg (54%) of N-(2,5-difluoro-4-(trifluoromethyl) phenyl)-5-(3-fluoropyridin-2-yl)-1H-pyrrole-3-sulfonamide (Cpd 148).

The following compounds were prepared in a similar manner (use of appropriate reagents and purification methods (including chiral HPLC or chiral SFC) known to the person skilled in the art) as described for Cpd 148: Cpd 026; Cpd 122; Cpd 123; Cpd 124; Cpd 125; Cpd 149; Cpd 150; Cpd 189; Cpd 197; Cpd 204; Cpd 280; Cpd 282; Cpd 302; Cpd 303; Cpd 304; Cpd 305; Cpd 310; Cpd 322; Cpd 332; Cpd 339; Cpd 344; Cpd 345; Cpd 346; Cpd 347; Cpd 348; Cpd 349; Cpd 350; Cpd 353; Cpd 354; Cpd 357; Cpd 369; Cpd 370; Cpd 371; Cpd 372; Cpd 373; Cpd 374; Cpd 379; Cpd 396; Cpd 403; Cpd 404; Cpd 405; Cpd 406; Cpd 419; Cpd 473; Cpd 515; Cpd 516; Cpd 528; Cpd 537 (from I-018); Cpd 560; Cpd 573 (from I-023); Cpd 575; Cpd 577; Cpd 580; Cpd 581; Cpd 592 (from I-023); Cpd 598; Cpd 606; Cpd 607; Cpd 610; Cpd 617; Cpd 618; Cpd 619; Cpd 628; Cpd 629; Cpd 634; Cpd 635; Cpd 637; Cpd 638; Cpd 639; Cpd 641; Cpd 642; Cpd 646; Cpd 647; Cpd 648; Cpd 649; Cpd 656; Cpd 657; Cpd 667 and Cpd 668.

### Synthesis of N-[2-methoxy-4-(trifluoromethyl)phenyl]-5-(2-pyridyl)-1H-pyrrole-3-sulfonamide (Cpd 154)

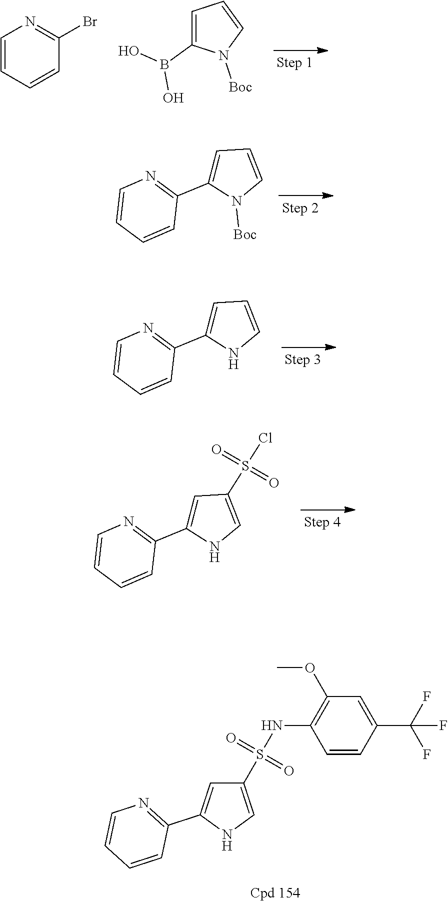 [SMILES: Brc1ccccn1 ; CC(C)(C)OC(=O)n1cccc1B(O)O ; CC(C)(C)OC(=O)n1cccc1-c1ccccn1 ; c1ccc(-c2ccc[nH]2)nc1 ; O=S(=O)(Cl)c1c[nH]c(-c2ccccn2)c1 ; COc1cc(C(F)(F)F)ccc1NS(=O)(=O)c1c[nH]c(-c2ccccn2)c1]

Step 1: To a mixture of 1-(tert-butoxycarbonyl) pyrrol-2-ylboronic acid (5.0 g, 23.7 mmol) and 2-bromopyridine (3.7 g, 23.7 mmol) in THF (110 mL) and H₂O (10 mL) were added Pd(PPh₃)₄ (1.37 g, 1.19 mmol) and K₂CO₃ (9.9 g, 71 mmol) at RT under Ar atmosphere. The RM was stirred for 18 h at 100° C. After cooling at RT, the RM was filtered and the solid was washed with DCM (3×100 mL). The filtrate was concentrated under reduced pressure. The residue was purified by FCC on silica gel using, as eluent PE/EtOAc (10/1) to afford 5.6 g (96%) of tert-butyl 2-(pyridin-2-yl) pyrrole-1-carboxylate. ¹H NMR (400 MHz, CDCl₃) δ ppm 8.61 (m, 1H), 7.72-7.59 (m, 1H), 7.46-7.28 (m, 2H), 7.19 (m, 1H), 6.41 (dd, 1H), 6.24 (m, 1H), 1.35 (s, 9H).

Step 2: To a solution of tert-butyl 2-(pyridin-2-yl) pyrrole-1-carboxylate (5 g, 20 mmol) in MeOH (100 mL) was added MeONa (5.5 g, 102 mmol) dropwise at RT under N₂. The RM was stirred for 12 h at 65° C. After cooling at RT, 10 mL water was added to the RM. The mixture was extracted with EtOAc (3×300 mL). The organic layers were combined, washed with brine (3×200 mL), dried over Na₂SO₄, filtrated, and concentrated under reduced pressure to afford 2.8 g (95%) of 2-(1H-pyrrol-2-yl) pyridine. ¹H NMR (300 MHz, CDCl₃) δ ppm 12.69 (s, 1H), 8.55 (d, 1H), 8.42-8.34 (m, 2H), 7.59 (d, 2H), 7.35 (d, 1H), 6.38 (m, 1H).

Step 3: To a mixture of 2-(1H-pyrrol-2-yl) pyridine (2.8 g, 19.4 mmol) was added chlorosulfonic acid (12.8 mL, 194 mmol) dropwise at RT under Ar atmosphere. The RM was stirred for 24 h at 0° C. The reaction was quenched with water at 0° C. The RM was extracted with DCM (3×200 mL). The combined organic layers were washed with brine (3×100 mL), dried over Na₂SO₄, filtered, and concentrated under reduced pressure to afford 2.3 g (49%) of 5-(pyridin-2-yl)-1H-pyrrole-3-sulfonyl chloride. ¹H NMR (300 MHz, CDCl₃) δ ppm 10.32 (s, 1H), 8.50 (m, 1H), 7.66 (m, 1H), 7.06 (m, 1H), 6.93 (m, 1H), 6.81-6.70 (m, 1H), 6.34 (m, 1H).

Step 4: To a solution of 2-methoxy-4-(trifluoromethyl)aniline (354 mg, 1.8 mmol) in pyridine (10 mL) was added 5-(pyridin-2-yl)-1H-pyrrole-3-sulfonyl chloride (300 mg, 1.2 mmol) at RT under Ar atmosphere. The RM was stirred for 12 h at 80° C. After cooling at RT, the RM was concentrated under reduced pressure. The residue was purified by R^{p} flash chromatography C18 (column: Gemini-NX C18 AXAI Packed, 21.2*150 mm 5 μm) using a gradient of MeCN (36 to 67%) in water (10 mM of NH₄HCO₃) to afford 140 mg (28%) of N-[2-methoxy-4-(trifluoromethyl)phenyl]-5-(2-pyridyl)-1H-pyrrole-3-sulfonamide (Cpd 154).

The following compounds were prepared in a similar manner (use of appropriate reagents and purification methods (including chiral HPLC or chiral SFC) known to the person skilled in the art) as described for Cpd 154: Cpd 157; Cpd 172; Cpd 173; Cpd 174; Cpd 178; Cpd 179; Cpd 181; Cpd 183; Cpd 185; Cpd 190; Cpd 196; Cpd 198; Cpd 200; Cpd 201; Cpd 202; Cpd 203; Cpd 205; Cpd 237; Cpd 239; Cpd 241; Cpd 246; Cpd 286; Cpd 311 (from I-001); Cpd 530; Cpd 531; Cpd 532 and Cpd 534.

### Synthesis of 5-phenyl-N-[6-(trifluoromethyl)-3-pyridyl]-1H-pyrrole-3-sulfonamide (Cpd 099) from I-004

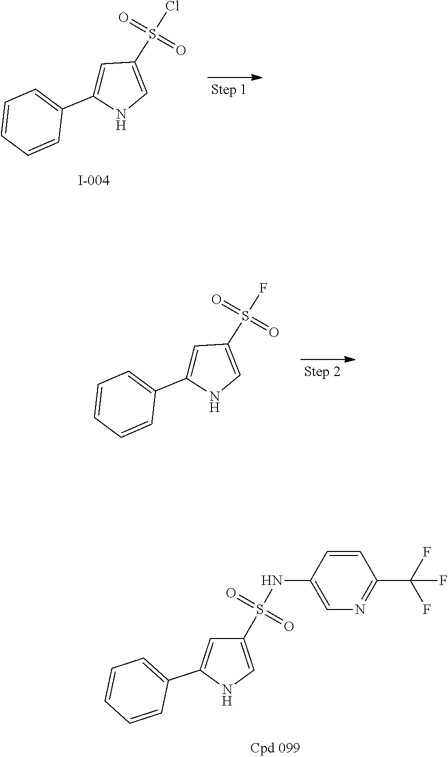 [SMILES: O=S(=O)(Cl)c1c[nH]c(-c2ccccc2)c1 ; O=S(=O)(F)c1c[nH]c(-c2ccccc2)c1 ; O=S(=O)(Nc1ccc(C(F)(F)F)nc1)c1c[nH]c(-c2ccccc2)c1]

Step 1: A mixture of 5-phenyl-1H-pyrrole-3-sulfonyl chloride (I-004) (400 mg, 1.6 mmol) and TBAF (3.3 mL, 3.3 mmol, 1M in THF) in THF (10 mL) was stirred for 16 h at RT. The RM was diluted with water (100 ml) and extracted with EtOAc (3×100 mL). The combined organic layers were washed with brine (100 mL), dried over Na₂SO₄, filtrated, and concentrated under reduced pressure. The residue was purified by preparative TLC (Eluent: hexane/EtOAc: 5/1) to afford 148 mg (40%) of 5-phenyl-1H-pyrrole-3-sulfonyl fluoride. ¹H NMR (300 MHz, DMSO-d6) δ ppm 12.83 (s, 1H), 8.03 (d, 1H), 7.82-7.72 (m, 2H), 7.63-7.38 (m, 2H), 7.38-7.26 (m, 1H), 7.18 (d, 1H).

Step 2: TMSNTf₂ (162 mg, 0.44 mmol) was added to a solution of 5-phenyl-1H-pyrrole-3-sulfonyl fluoride (100 mg, 0.44 mmol) and 5-amino-2-(trifluoromethyl)pyridine (147 mg, 0.89 mmol) in dry Pyridine (2.2 mL) under inert atmosphere. The RM was refluxed overnight until completion. After cooling, the RM was diluted with DCM and partitioned with water. Aqueous layer was back extracted again with DCM. Combined organic layers were dried over MgSO₄, filtered, and concentrated under reduced pressure. The residue was purified by FCC over silica gel using a gradient of EtOAc (0 to 100%) in PE to afford 102 mg (62%) of 5-phenyl-N-[6-(trifluoromethyl)-3-pyridyl]-1H-pyrrole-3-sulfonamide (Cpd 099).

The following compounds were prepared in a similar manner (use of appropriate reagents and purification methods (including chiral HPLC or chiral SFC) known to the person skilled in the art) as described for Cpd 099: Cpd 100; Cpd 101 and Cpd 112.

### Synthesis of N-[6-(difluoromethoxy)-2-fluoro-3-pyridyl]-5-phenyl-1H-pyrrole-3-sulfonamide (Cpd 188) from I-004

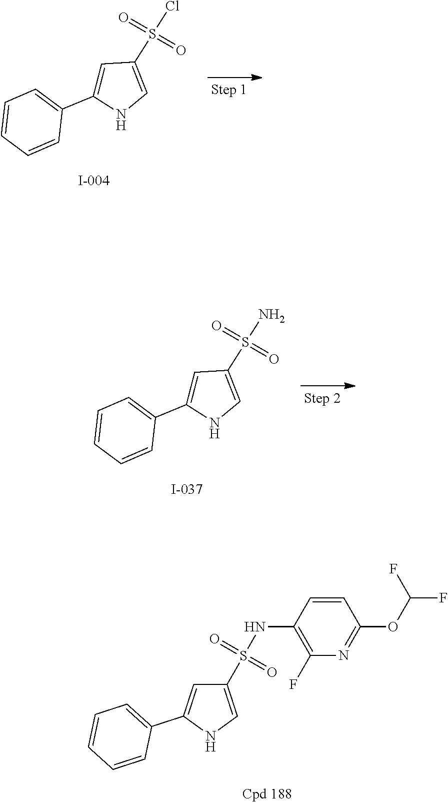 [SMILES: O=S(=O)(Cl)c1c[nH]c(-c2ccccc2)c1 ; NS(=O)(=O)c1c[nH]c(-c2ccccc2)c1 ; O=S(=O)(Nc1ccc(OC(F)F)nc1F)c1c[nH]c(-c2ccccc2)c1]

Step 1: To a stirred solution of 5-phenyl-1H-pyrrole-3-sulfonyl chloride (I-004) (500 mg, 2.1 mmol) in THF (6 mL) was added aq. NH₃ (6 mL) at 0° C. The RM was stirred for 1 h. After completion, the RM was concentrated under reduced pressure, diluted with water, extracted with EtOAc, dried over Na₂SO₄, filtrated to afford 150 mg (33%) of 5-phenyl-1H-pyrrole-3-sulfonamide (I-037), which was used without further purification.

Step 2: To a degassed solution of 5-phenyl-1H-pyrrole-3-sulfonamide (150 mg, 0.68 mmol) in dry MeCN (5 ml) were added 3-bromo-6-(difluoromethoxy)-2-fluoropyridine (195 mg, 0.8 mmol), K₂CO₃ (233 mg, 1.7 mmol), CuI (6.4 mg, 0.03 mmol) and trans-N,N-dimethylcyclohexane-1,2-diamine (0.05 ml, 0.34 mmol). After 16 h at 80° C., the RM was filtered through celite bed and filtrate was concentrated under reduced pressure. The residue was purified by FCC over silica gel using a gradient of EtOAc (0 to 50%) in hexane. The residue was purified by Preparative HPLC on Waters auto purification instrument with a YMC Actus Triart C18 (250×20 mm, 5p) column, operating at RT and flow rate of 16 mL/min. Mobile phase: A=20 mM NH₄HCO₃ in water, B=MeCN; Gradient Profile: Mobile phase initial composition of 80% A and 20% B, then 75% A and 25% B in 3 min, then to 40% A and 60% B in 22 min., then to 5% A and 95% B in 23 min., held this composition up to 25 min. The purification afforded 70 mg (27%) of N-[6-(difluoromethoxy)-2-fluoro-3-pyridyl]-5-phenyl-1H-pyrrole-3-sulfonamide (Cpd 188).

The following compounds were prepared in a similar manner (use of appropriate reagents and purification methods (including chiral HPLC or chiral SFC) known to the person skilled in the art) as described for Cpd 188: Cpd 186; Cpd 328; Cpd 601 (from I-024); Cpd 631; Cpd 644 (from I-025); Cpd 645 (from I-028) and Cpd 650 (from I-028).

### Synthesis of N-(4-cyano-5-fluoro-2-methoxyphenyl)-5-phenyl-1H-pyrrole-3-sulfonamide (Cpd

063) from I-004

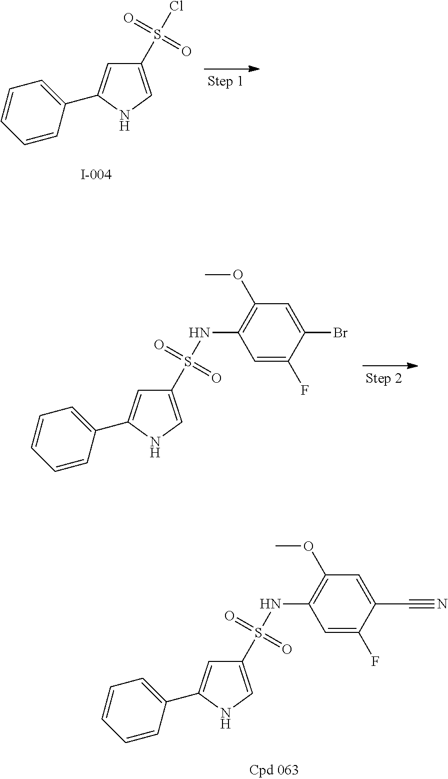 [SMILES: O=S(=O)(Cl)c1c[nH]c(-c2ccccc2)c1 ; COc1cc(Br)c(F)cc1NS(=O)(=O)c1c[nH]c(-c2ccccc2)c1 ; COc1cc(C#N)c(F)cc1NS(=O)(=O)c1c[nH]c(-c2ccccc2)c1]

Step 1: To a solution of NaH (60% in mineral oil) (531 mg, 13.28 mmol,) in THF (5 mL) was added 4-bromo-5-fluoro-2-methoxyaniline (1.1 g, 4.98 mmol) at 0° C. The RM was stirred at RT for 1 h. To the RM was added 5-phenyl-1H-pyrrole-3-sulfonyl chloride (I-004) (800 mg) in THF (3 ml) dropwise at 0° C. The RM was stirred for 2 h at RT. The RM was quenched by MeOH (1 ml). The volatiles were removed under reduced pressure. The residue was purified by R^{p} FCC on C18 gel using a gradient of MeCN (30 to 70%) in water (0.5% NH₄HCO₃) to afford 456 mg (32%) of N-(4-bromo-5-fluoro-2-methoxyphenyl)-5-phenyl-1H-pyrrole-3-sulfonamide.

Step 2: To a solution of N-(4-bromo-5-fluoro-2-methoxyphenyl)-5-phenyl-1H-pyrrole-3-sulfonamide (300 mg, 0.71 mmol) in DMF (5 mL) were added Zn(CN)₂ (166 mg, 1.41 mmol), Pd₂(dba)₃ (65 mg, 0.07 mmol) and XPhos (17 mg, 0.04 mmol). The RM was stirred for 4 h at 120° C. under N₂. After cooling down at RT, the RM was concentrated under reduced pressure. The residue was purified by FCC over silica gel using a gradient of EtOAc (10 to 20%) in PE. The residue was purified again by R^{p} flash chromatography on C18 gel using a gradient of MeCN (40 to 60%) in water with 0.1% FA to afford 20 mg (8%) of N-(4-cyano-5-fluoro-2-methoxyphenyl)-5-phenyl-1H-pyrrole-3-sulfonamide (Cpd 063).

### Synthesis of N-(4-fluorothiophene-2-yl)-5-phenyl-1H-pyrrole-3-sulfonamide (Cpd 593)

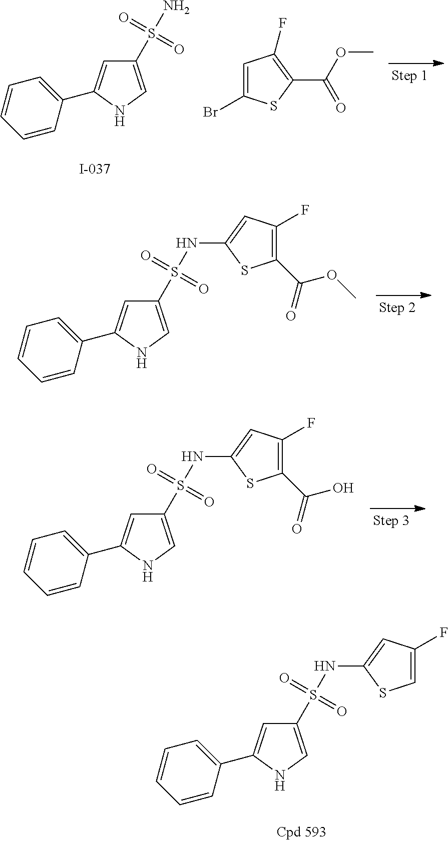 [SMILES: NS(=O)(=O)c1c[nH]c(-c2ccccc2)c1 ; COC(=O)c1sc(Br)cc1F ; COC(=O)c1sc(NS(=O)(=O)c2c[nH]c(-c3ccccc3)c2)cc1F ; O=C(O)c1sc(NS(=O)(=O)c2c[nH]c(-c3ccccc3)c2)cc1F ; O=S(=O)(Nc1cc(F)cs1)c1c[nH]c(-c2ccccc2)c1]

Step 1: 5-phenyl-1H-pyrrole-3-sulfonamide (I-037) (450 mg, 2.025 mmol) and methyl 5-bromo-3-fluorothiophene-2-carboxylate (481.69 mg, 2.025 mmol) were taken in a sealed tube. MeCN (3.0 mL) was added and the reaction mixture was degassed under Argon for 15 minutes. K₂CO₃ (698.51 mg, 5.062 mmol), CuI (131.10 mg, 0.688 mmol), and trans-N,N′-Dimethyl-cyclohexane-1,2-diamine (230.39 mg, 1.62 mmol) were added and the reaction mixture was heated at 120° C. for 16 hours. Upon completion, solvent was evaporated under reduced pressure and crude thus obtained was purified by FCC on silica gel using a gradient of EtOAc (0-10%) in Hexane to afford 340 mg (44%) of methyl 3-fluoro-5-((5-phenyl-1H-pyrrole)-3-sulfonamido)thiophene-2-carboxylate. ¹H NMR (400 MHz, DMSO-d6): δ ppm 12.21 (s, 1H), 11.55 (br s, 1H), 7.66 (d, 2H), 7.53 (s, 1H), 7.39 (t, 2H), 7.26 (t, 1H), 6.76 (s, 1H), 6.42 (s, 1H), 3.71 (s, 3H).

Step 2: To a stirred solution of methyl 3-fluoro-5-((5-phenyl-1H-pyrrole)-3-sulfonamido)thiophene-2-carboxylate (220.0 mg, 0.578 mmol) in THF/water (4:1, 5.0 mL) was added LiOH·H₂O (121.33 mg, 2.892 mmol) at 0° C. Reaction mixture was heated at 60° C. for 16 hours. After completion, reaction mixture was quenched with water and extracted with ethyl acetate. The aqueous phase was acidified with 2N HCl (pH˜2.0) and extracted with ethyl acetate. Combined organic extracts were washed with brine, dried over anhydrous Na₂SO₄, filtered and concentrated under reduced pressure to afford 200 mg (94%) of 3-fluoro-5-((5-phenyl-1H-pyrrole)-3-sulfonamido)thiophene-2-carboxylic acid. LCMS (ES−, m/z) [M−H]⁻=365.0.

Step 3: To a stirred solution of 3-fluoro-5-((5-phenyl-1H-pyrrole)-3-sulfonamido)thiophene-2-carboxylic acid (180.0 mg, 0.491 mmol) in DMSO (3.0 mL) were added AcOH (0.3 mL) and Silver carbonate (27.094 mg, 0.098 mmol). The resulting reaction mixture was heated at 80° C. for 2 hours. Reaction mixture was diluted with ice-cold water and extracted with ethyl acetate for several times. The organic phases were dried over anhydrous Na₂SO₄, filtered and concentrated under reduced pressure. Crude thus obtained was purified by FCC on silica gel using a gradient of EtOAc (5-50%) in hexane to afford 40 mg (25%) of N-(4-fluorothiophene-2-yl)-5-phenyl-1H-pyrrole-3-sulfonamide (Cpd 593).

### Synthesis of N-(4-cyano-2-fluorophenyl)-5-cyclopentyl-1H-pyrrole-3-sulfonamide (Cpd 018)

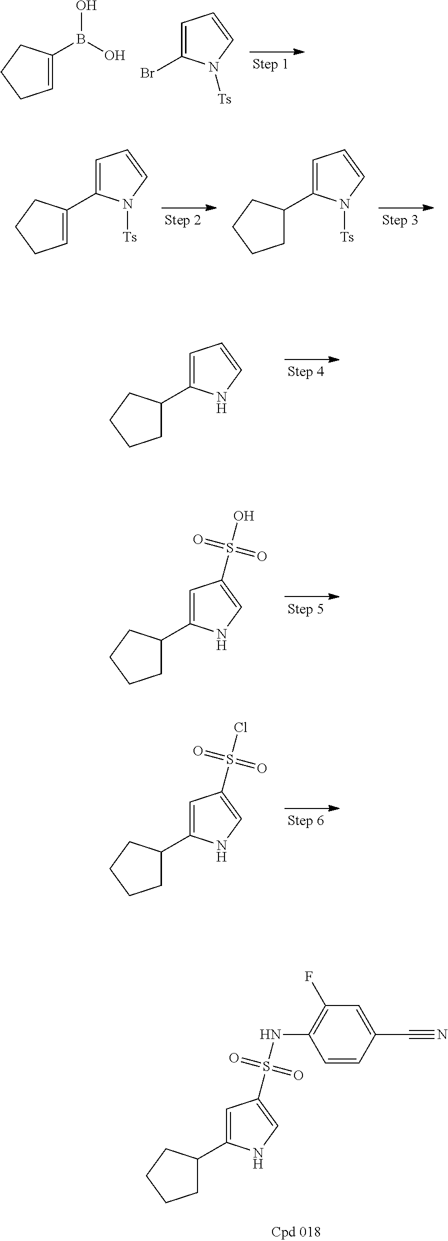

Step 1: To a mixture of 2-bromo-1-(4-methyl benzenesulfonyl)pyrrole (5.0 g, 16.6 mmol) and cyclopent-1-en-1-ylboronic acid (3.7 g, 33.3 mmol) in dioxane (30 mL) and H₂O (1.5 mL) were added CsF (7.6 g, 50 mmol) and Pd(dppf)Cl₂ (0.61 g, 0.83 mmol). The RM was stirred for 3 h at 100° C. under N₂ atmosphere. The RM was concentrated under reduced pressure. The residue was purified by FCC over silica gel using as eluent EtOAc/PE (1/100) to afford 2.8 g (59%) of 2-(cyclopent-1-en-1-yl)-1-(4-methylbenzenesulfonyl)pyrrole. ¹H NMR (400 MHz, DMSO-d6) δ 7.88-7.81 (m, 1H), 7.64-7.52 (m, 2H), 7.49-7.38 (m, 4H), 7.38-7.30 (m, 1H), 6.37-6.30 (m, 2H), 6.20 (dd, 1H), 5.81 (q, 1H), 2.46-2.35 (m, 9H), 1.83 (p, 2H).

Step 2: A solution of 2-(cyclopent-2-en-1-yl)-1-(4-methyl benzenesulfonyl)pyrrole (2.7 g, 9.4 mmol) and Pd/C (270 mg) in DCM (50 mL) was stirred for 5 h at RT under hydrogen atmosphere. The RM was filtered through a Celite pad, the filter cake was washed with DCM (300 mL). The filtrate was concentrated under reduced pressure to afford 2.7 g (100%) 2-cyclopentyl-1-(4-methylbenzenesulfonyl)pyrrole.

Step 3: A solution of 2-cyclopentyl-1-(4-methylbenzenesulfonyl)pyrrole (2.80 g, 9.68 mmol) and NaOH (3.9 g, 96.76 mmol) in MeOH/H₂O (30/10 mL) was stirred overnight at 80° C. The RM was concentrated under reduced pressure. The residue was dissolved in EtOAc (100 mL), and then washed with water (50 mL), and brine (50 mL), dried over Na₂SO₄, filtered, and concentrated under reduced pressure to afford 1.10 g (84%) of 2-cyclopentyl-1H-pyrrole.

Step 4: To a solution of 2-cyclopentyl-1H-pyrrole (450 mg, 3.33 mmol) in MeCN (10 mL) was added Py·SO₃ (636 mg, 3.99 mmol). The RM was stirred for 3 h at 120° C. The RM was concentrated under reduced pressure. The residue was dissolved in water (50 mL) and washed with CHCl₃ (50 mL×3). The aqueous phase was concentrated under reduced pressure to afford 900 mg of 5-cyclopentyl-1H-pyrrole-3-sulfonic acid, which was used without further purification.

Step 5: A solution of 5-cyclopentyl-1H-pyrrole-3-sulfonic acid (850 mg, 3.95 mmol) and POCl₃ (1.2 g, 7.9 mmol) in MeCN (10 mL) was stirred 3 h at 70° C. under N₂ atmosphere. The RM was then poured into the ice-water. And then extracted with CHCl₃ (3×50 mL). The organic layers were combined, dried over Na₂SO₄, filtered, and concentrated under reduced pressure to afford 550 mg of 5-cyclopentyl-1H-pyrrole-3-sulfonyl chloride, which was used without further purification.

Step 6: To a solution of 5-cyclopentyl-1H-pyrrole-3-sulfonyl chloride (850 mg, 3.6 mmol) and 4-amino-3-fluorobenzonitrile (990 mg, 7.3 mmol) in MeCN (8 mL) was added pyridine (2.88 g, 36.4 mmol) at RT. The RM was stirred overnight at RT under N₂ atmosphere. The RM was concentrated under reduced pressure. The residue was purified by R^{p} FCC on C18 gel using a gradient of MeCN (0 to 100%) in water with 0.1% FA. The residue was further purified by preparative TLC (PE/EtOAc 3:1) to afford 38 mg (4%) of N-(4-cyano-2-fluorophenyl)-5-cyclopentyl-1H-pyrrole-3-sulfonamide (Cpd 018).

The following compounds were prepared in a similar manner (use of appropriate reagents and purification methods (including chiral HPLC or chiral SFC) known to the person skilled in the art) as described for Cpd 018: Cpd 421 (from I-031); Cpd 423 (from I-031), Cpd 424 (from I-032); Cpd 578 (from I-033 and I-017) and Cpd 621 (from I-034).

### Synthesis of 5-(4-fluorophenyl)-N-(2,4,5-trifluorophenyl)-1H-pyrrole-3-sulfonamide (Cpd 109)

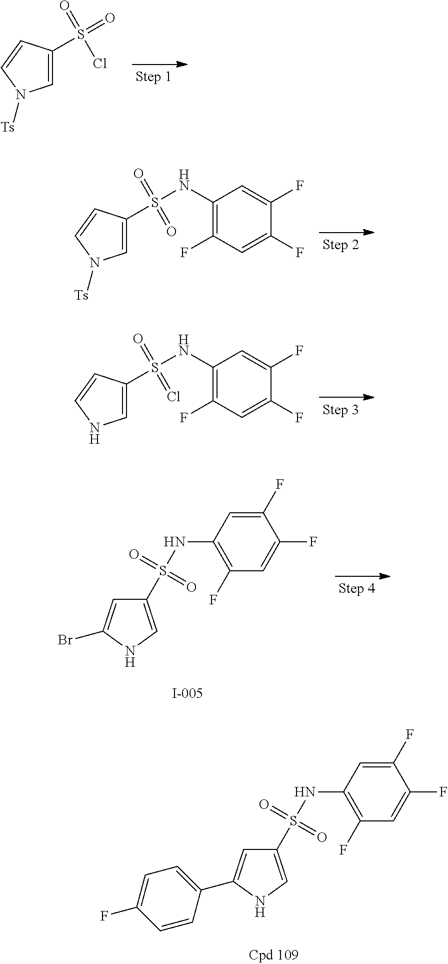

Step 1: To a solution of 1-tosyl-1H-pyrrole-3-sulfonyl chloride (2.0 g, 6.25 mmol) in dry MeCN (5 mL) were added 2,4,5-trifluoroaniline (2.46 g, 12.5 mmol) and pyridine (0.76 ml, 9.38 mmol) under N₂. The RM was stirred at RT for 8 h. The RM was concentrated under reduced pressure and diluted with water. The aqueous layer was extracted thrice with EtOAc. The organic layers were combined; dried over Na₂SO₄, filtered, and concentrated under reduced pressure. The residue was purified by FCC over silica gel using a gradient of EtOAc (0 to 20%) in hexane to afford 2.5 g (93%) of 1-tosyl-N-(2,4,5-trifluorophenyl)-1H-pyrrole-3-sulfonamide. ¹H NMR (400 MHz, DMSO): δ ppm 10.23 (s, 1H), 7.92 (d, 2H), 7.77 (s, 1H), 7.5-7.45 (m, 4H), 7.25-7.19 (m, 1H), 6.49 (s, 1H), 2.4 (s, 3H).

Step 2: To a solution of N-(2,5-difluorophenyl)-1-tosyl-1H-pyrrole-3-sulfonamide (2.5 g, 5.81 mmol) in a mixture of MeOH (20 mL) and H₂O (10 ml) was added LiOH·H₂O (696 mg, 29.07 mmol) portion wise at 0° C. The RM was stirred for 1 h at RT. The RM was concentrated under reduced pressure. The residue was diluted in water and the pH was adjusted to ˜7 by addition of 1N HCl aq. solution at 0° C. Then, the RM was extracted with DCM. The organic layers were combined, dried over Na₂SO₄, filtered, and concentrated under reduced pressure to afford 1.5 g (93%) of N-(2,4,5-trifluorophenyl)-1H-pyrrole-3-sulfonamide. ¹H NMR (400 MHz, DMSO-d6): δ ppm 11.58 (s, 1H), 9.83 (s, 1H), 7.55-7.49 (m, 1H), 7.3-7.24 (m, 2H), 6.85 (s, 1H), 6.25 (s, 1H).

Step 3: To a solution of N-(2,4,5-trifluorophenyl)-1H-pyrrole-3-sulfonamide (900 mg, 3.26 mmol) in DMF (20 ml) was added, at −50° C., NBS (581 mg, 3.26 mmol). The RM was stirred at −50° C. for 2 h. The RM was allowed to warm up to RT and stirred overnight. The RM was diluted with cold water, extracted with EtOAc, dried over Na₂SO₄, filtrated, and concentrated under reduced pressure. The residue was purified by FCC over silica gel using a gradient of EtOAc (0 to 80%) in hexane to afford 350 mg (30%) of 5-bromo-N-(2,4,5-trifluorophenyl)-1H-pyrrole-3-sulfonamide (I-005). ¹H NMR (400 MHz, DMSO-d6): δ ppm 12.36 (s, 1H), 9.97 (s, 1H), 7.6-7.53 (m, 1H), 7.31-7.26 (m, 2H), 6.33 (s, 1H).

Step 4: To a solution of 5-bromo-N-(2,4,5-trifluorophenyl)-1H-pyrrole-3-sulfonamide (200 mg, 0.56 mmol) and (4-fluorophenyl)boronic acid (157 mg, 1.13 mmol) in Toluene (5 ml) and water (0.2 ml) was added Na₂CO₃ (89.5 mg, 0.845 mmol). The RM was degassed with N₂ before the addition of Pd(PPh₃)₄ (6.51 mg, 0.006 mmol). The RM was stirred for 8 h at 100° C. The RM was concentrated under reduced pressure. The residue was purified by FCC over silica gel using a gradient of EtOAc (0 to 70%) in hexane. The residue was purified by preparative HPLC on Waters auto purification instrument with a YMC Actus Triart C18 (250×20 mm, 5p) column, operating at RT and flow rate of 16 mL/min. Mobile phase: A=20 mM NH₄HCO₃ in water, B=MeCN; Gradient Profile: Mobile phase initial composition of 70% A and 30% B, then 60% A and 40% B in 3 min, then to 30% A and 70% B in 20 min., then to 5% A and 95% B in 21 min., held this composition up to 23 min. The purification afforded 60 mg (29%) of 5-(4-fluorophenyl)-N-(2,4,5-trifluorophenyl)-1H-pyrrole-3-sulfonamide (Cpd 109).

The following compounds were prepared in a similar manner (use of appropriate reagents and purification methods (including chiral HPLC or chiral SFC) known to the person skilled in the art) as described for Cpd 109: Cpd 071; Cpd 078; Cpd 079; Cpd 080; Cpd 081; Cpd 082; Cpd 083; Cpd 084; Cpd 085; Cpd 086; Cpd 087; Cpd 088; Cpd 089 (from I-002); Cpd 091; Cpd 093; Cpd 095; Cpd 096; Cpd 097; Cpd 102; Cpd 103; Cpd 104; Cpd 105; Cpd 106; Cpd 107; Cpd 108; Cpd 110; Cpd 111; Cpd 113; Cpd 114; Cpd 117; Cpd 118; Cpd 119; Cpd 120; Cpd 121; Cpd 131; Cpd 160; Cpd 224 (from I-002); Cpd 226; Cpd 227 (from I-002); Cpd 234; Cpd 249; Cpd 250; Cpd 251; Cpd 252; Cpd 253; Cpd 254; Cpd 255; Cpd 256; Cpd 257; Cpd 258; Cpd 259; Cpd 260; Cpd 261; Cpd 262; Cpd 263; Cpd 264; Cpd 265; Cpd 266; Cpd 267; Cpd 268; Cpd 269; Cpd 270; Cpd 272; Cpd 273; Cpd 274; Cpd 283; Cpd 284; Cpd 285; Cpd 287; Cpd 288; Cpd 289; Cpd 290; Cpd 291; Cpd 292; Cpd 293; Cpd 294; Cpd 295; Cpd 296; Cpd 297; Cpd 298; Cpd 299; Cpd 300; Cpd 301; Cpd 314; Cpd 315; Cpd 316; Cpd 318; Cpd 319; Cpd 320; Cpd 321; Cpd 323; Cpd 324; Cpd 325; Cpd 327; Cpd 329; Cpd 330; Cpd 331; Cpd 335; Cpd 336; Cpd 337; Cpd 351; Cpd 352; Cpd 355; Cpd 356; Cpd 358; Cpd 361; Cpd 362; Cpd 363; Cpd 365; Cpd 366; Cpd 394; Cpd 397; Cpd 442; Cpd 443; Cpd 456, Cpd 505; Cpd 517; Cpd 519; Cpd 540; Cpd 552; Cpd 553; Cpd 596; Cpd 615; Cpd 652; Cpd 654 and Cpd 663 (from I-029).

### Synthesis of N-(4-cyano-2-fluorophenyl)-5-(3-fluoropyridin-2-yl)-1H-pyrrole-3-sulfonamide (Cpd 090)

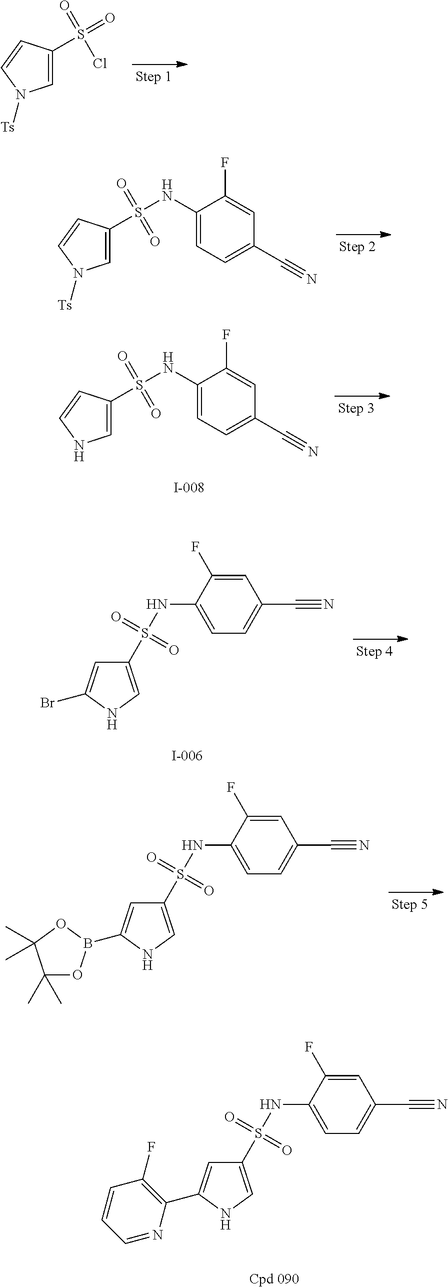 [SMILES: Cc1ccc(S(=O)(=O)n2ccc(S(=O)(=O)Cl)c2)cc1 ; Cc1ccc(S(=O)(=O)n2ccc(S(=O)(=O)Nc3ccc(C#N)cc3F)c2)cc1 ; N#Cc1ccc(NS(=O)(=O)c2cc[nH]c2)c(F)c1 ; N#Cc1ccc(NS(=O)(=O)c2c[nH]c(Br)c2)c(F)c1 ; CC1(C)OB(c2cc(S(=O)(=O)Nc3ccc(C#N)cc3F)c[nH]2)OC1(C)C ; N#Cc1ccc(NS(=O)(=O)c2c[nH]c(-c3ncccc3F)c2)c(F)c1]

Step 1: To a solution of 4-amino-3-fluorobenzonitrile (29.4 g, 216 mmol) in MeCN (300 mL) were added Pyridine (42.8 g, 541 mmol) dropwise at 0° C., followed by 1-(benzenesulfonyl)pyrrole-3-sulfonyl chloride (33 g, 108 mmol) in MeCN (50 ml). The RM was stirred overnight at RT. The solvent was removed under reduced pressure. The residue was purified by FCC over silica gel using a gradient of EtOAc (10 to 50%) in PE to afford 15 g (35%) of 1-(benzenesulfonyl)-N-(4-cyano-2-fluorophenyl)pyrrole-3-sulfonamide. ¹H NMR (300 MHz, DMSO-d6): δ 10.76 (s, 1H), 8.00-8.21 (m, 3H), 7.73-7.85 (m, 2H), 7.62-7.72 (m, 2H), 7.40-7.60 (m, 3H), 6.55 (s, 1H).

Step 2: To a solution of 1-(benzenesulfonyl)-N-(4-cyano-2-fluorophenyl)pyrrole-3-sulfonamide (15 g, 38 mmol) in MeOH (100 mL) and H₂O (50 mL) was added LiOH (4.58 g, 191 mmol) at 0° C. The RM was stirred for 1 h at RT. The RM was adjusted to pH 7 using an aq. solution of 1N HCl. The solution was concentrated under reduced pressure. The residue was purified by R^{p} FCC on C18 gel using a gradient of MeCN (20 to 25%) in water with 0.1% FA to afford 9.2 g (91%) of (N-(4-cyano-2-fluorophenyl)-1H-pyrrole-3-sulfonamide (I-008) (9.20 g, 90.7%). ¹H NMR (300 MHz, DMSO-d6): (511.64 (s, 1H), 10.43 (s, 1H), 7.81 (dd, 1H), 7.52-7.65 (m, 2H), 7.41 (s, 1H), 6.87 (s, 1H), 6.31 (s, 1H).

Step 3: To a solution of N-(4-cyano-2-fluorophenyl)-1H-pyrrole-3-sulfonamide (I-008) (1.00 g, 3.77 mmol) in DMF (50 mL) was added NBS (671 mg, 3.77 mmol) at −50° C. The RM was stirred at −50° C. for 2 h, then warmed to RT and stirred overnight at RT. The RM was dissolved in EtOAc (100 mL), washed with water (50 mL), and brine (50 mL), dried over Na₂SO₄, filtrated, and concentrated under reduced pressure. The residue was purified by R^{p} FCC on C18 gel using a gradient of MeCN (50 to 55%) in water with 0.1% FA to afford 477 mg (37%) of 5-bromo-N-(4-cyano-2-fluorophenyl)-1H-pyrrole-3-sulfonamide (I-006). ¹H NMR (300 MHz, DMSO-d6): δ 12.47 (s, 1H), 10.55 (s, 1H), 7.80-7.90 (m, 1H), 7.50-7.70 (m, 2H), 7.45-7.50 (m, 1H), 6.40 (s, 1H).

Step 4: To a solution of 5-bromo-N-(4-cyano-2-fluorophenyl)-1H-pyrrole-3-sulfonamide (400 mg, 1.16 mmol) in dioxane (10 mL) and DMSO (0.2 mL) were added bis(pinacolato)diboron (442 mg, 1.74 mmol, 1.50 equiv), AcOK (228 mg, 2.32 mmol), Pd(dppf)Cl₂ (84 mg, 0.116 mmol) at RT. The RM was stirred for 2 h at 100° C. under N₂. The RM was dissolved with EtOAc (200 mL), washed with H₂O (100 mL), dried over Na₂SO₄, filtrated, and concentrated under reduced pressure to afford 500 mg of N-(4-cyano-2-fluorophenyl)-5-(4,4,5,5-tetramethyl-1,3,2-dioxaborolan-2-yl)-1H-pyrrole-3-sulfonamide, which was used without further purification.

Step 5: To a solution of N-(4-cyano-2-fluorophenyl)-5-(4,4,5,5-tetramethyl-1,3,2-dioxaborolan-2-yl)-1H-pyrrole-3-sulfonamide (500 mg, 1.27 mmol) in dioxane (20 mL) and H₂O (1 mL) were added 2-bromo-3-fluoropyridine (224 mg, 1.27 mmol), CsF (579 mg, 3.81 mmol), Pd(dppf)Cl₂ (93 mg, 0.12 mmol). The RM was stirred at 100° C. overnight under N₂. The volatiles were removed under reduced pressure. The residue was purified by FCC over silica gel using a gradient of EtOAc (10 to 50%) in PE. The residue was further purified by Prep-HPLC on a XBridge Prep C18 OBD Column (19×150 mm, 5 μm); Mobile Phase A: Water (0.1% FA), Mobile Phase B: MeCN; Flow rate: 25 mL/min; Gradient: 37% to 55% of B in 8 min. The purification afforded 15 mg (4%) of N-(4-cyano-2-fluorophenyl)-5-(3-fluoropyridin-2-yl)-1H-pyrrole-3-sulfonamide (Cpd 090).

The following compounds were prepared in a similar manner (use of appropriate reagents and purification methods (including chiral HPLC or chiral SFC) known to the person skilled in the art) as described for Cpd 090: Cpd 235; Cpd 243 (from I-003); Cpd 244; Cpd 247; Cpd 275; Cpd 277; Cpd 278; Cpd 279; Cpd 281; Cpd 489; Cpd 498; Cpd 499; Cpd 500; Cpd 502; Cpd 503; Cpd 504; Cpd 507; Cpd 542 (from I-022); Cpd 543; Cpd 555; Cpd 556; Cpd 557; Cpd 565; Cpd 566; Cpd 567; Cpd 572; Cpd 599; Cpd 600; Cpd 603; Cpd 604; Cpd 612; Cpd 614; Cpd 620, Cpd 633; Cpd 636; Cpd 658; Cpd 659; Cpd 660; Cpd 661 and Cpd 666.

### Synthesis of N-(2,5-difluoro-4-(trifluoromethyl)phenyl)-5-(furan-3-yl)-1H-pyrrole-3-sulfonamide (Cpd 276)

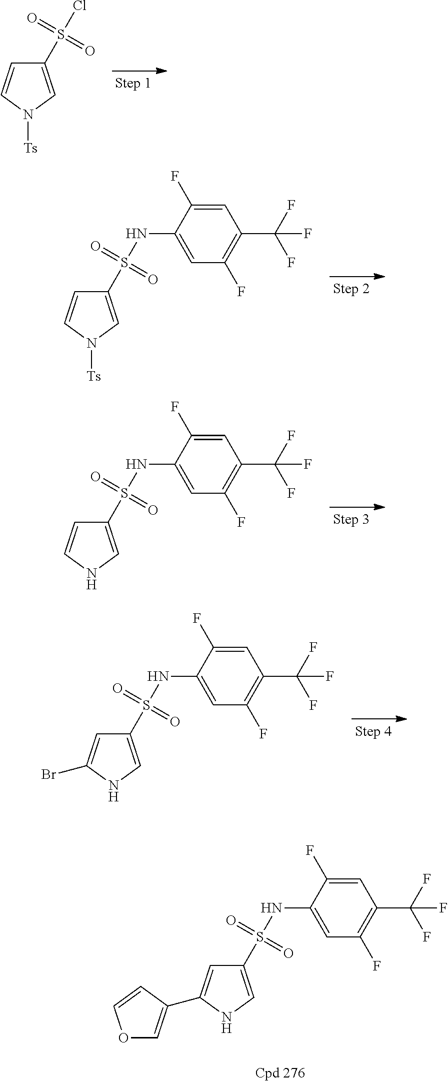 [SMILES: Cc1ccc(S(=O)(=O)n2ccc(S(=O)(=O)Cl)c2)cc1 ; Cc1ccc(S(=O)(=O)n2ccc(S(=O)(=O)Nc3cc(F)c(C(F)(F)F)cc3F)c2)cc1 ; O=S(=O)(Nc1cc(F)c(C(F)(F)F)cc1F)c1cc[nH]c1 ; O=S(=O)(Nc1cc(F)c(C(F)(F)F)cc1F)c1c[nH]c(Br)c1 ; O=S(=O)(Nc1cc(F)c(C(F)(F)F)cc1F)c1c[nH]c(-c2ccoc2)c1]

Step 1: A mixture of 1-(4-methylbenzenesulfonyl) pyrrole-3-sulfonyl chloride (1.0 g, 3.13 mmol) and 2,5-difluoro-4-(trifluoromethyl) aniline (925 mg, 4.69 mmol) in pyridine (15 mL) was stirred for 12 h at 80° C. under nitrogen atmosphere. The mixture was allowed to cool down to RT and was concentrated under reduced pressure. The residue was purified by FCC on silica gel eluted with EtOAc/PE (1:3) to afford 1.2 g (80%) of N-[2,5-difluoro-4-(trifluoromethyl)phenyl]-1-(4-methylbenzenesulfonyl)pyrrole-3-sulfonamide. ¹H NMR (400 MHz, CHCl₃) δ 7.78-7.71 (m, 3H), 7.42 (dd, J=11.1, 6.3 Hz, 1H), 7.36-7.31 (m, 2H), 7.25 (dd, J=9.9, 6.1 Hz, 1H), 7.15 (dd, J=3.4, 2.3 Hz, 1H), 6.48 (dd, J=3.4, 1.7 Hz, 1H), 2.43 (s, 3H).

Step 2: A solution of N-[2,5-difluoro-4-(trifluoromethyl) phenyl]-1-(4-m ethylbenzenesulfonyl) pyrrole-3-sulfonamide (1 g, 2.08 mmol) and LiOH (249.24 mg, 10.41 mmol) in MeOH (20 mL) was stirred for 1 h at RT under nitrogen atmosphere. The solvent was removed under reduced pressure and the resulting residue was purified by FCC on silica gel eluted with EtOAc/PE (2:5) to afford 620 mg (91%) of N-[2,5-difluoro-4-(trifluoromethyl)phenyl]-1H-pyrrole-3-sulfonamide. 1H NMR (400 MHz, DMSO-d6) δ 11.70 (s, 1H), 10.65 (s, 1H), 7.71 (m, 1H), 7.52 (m, 1H), 7.45 (dd, J=12.3, 6.3 Hz, 1H), 6.90 (m, 1H), 6.39 (m, 1H).

Step 3: To a stirred solution of N-[2,5-difluoro-4-(trifluoromethyl) phenyl]-1H-pyrrole-3-sulfonamide (500 mg, 1.53 mmol) in DMF (20 mL) was added NBS (272.8 mg, 1.53 mmol) dropwise at −50° C. under argon atmosphere. The reaction mixture was allowed to warm up and was stirred for 16 h at RT under argon atmosphere. The mixture was concentrated under reduced pressure and the residue obtained was purified by FCC on silica gel eluted with EtOAc/PE (1:4) to afford 300 mg (48%) of 5-bromo-N-[2,5-difluoro-4-(trifluoromethyl) phenyl]-1H-pyrrole-3-sulfonamide. ¹H NMR (300 MHz, DMSO-d6) δ 12.51 (s, 1H), 10.75 (s, 1H), 7.74 (dd, J=10.3, 6.7 Hz, 1H), 7.61 (dd, J=3.0, 1.8 Hz, 1H), 7.45 (dd, J=12.2, 6.3 Hz, 1H), 6.45 (dd, J=2.5, 1.8 Hz, 1H).

Step 4: To a stirred mixture of 5-bromo-N-[2,5-difluoro-4-(trifluoromethyl)phenyl]-1H-pyrrole-3-sulfonamide (300 mg, 0.74 mmol) and furan-3-ylboronic acid (165.8 mg, 1.48 mmol) in 1,4-dioxane (10 mL) and water (0.5 mL) were added Pd(dppf)Cl₂ (54.2 mg, 0.074 mmol) and CsF (225 mg, 1.48 mmol) at RT under nitrogen atmosphere. The resulting mixture was stirred for 3 h at 100° C. under nitrogen atmosphere. The mixture was allowed to cool down to RT and was concentrated under vacuum. The residue was purified by preparative HPLC on a Gemini-NX C18 AXIA™ Packed column (21.2×150 mm, 5 μm) operating with a flow rate of 16 mL/min; Mobile Phase A: Water(0.1% FA); Mobile Phase B: MeCN; Gradient profile: 45% B to 71% B in 7 min, 71% B. The purification afforded 110 mg (38%) of N-(2,5-difluoro-4-(trifluoromethyl)phenyl)-5-(furan-3-yl)-1H-pyrrole-3-sulfonamide.

The following compounds were prepared in a similar manner (use of appropriate reagents and purification methods (including chiral HPLC or chiral SFC) known to the person skilled in the art) as described for Cpd 276: Cpd 393, Cpd 441; Cpd 445; Cpd 457, Cpd 458, Cpd 462; Cpd 465, Cpd 474, Cpd 475, Cpd 476, Cpd 487, Cpd 488, Cpd 506; Cpd 518 (from I-021); Cpd 521; Cpd 538; Cpd 539; Cpd 554; Cpd 559; Cpd 562; Cpd 563; Cpd 564; Cpd 570; Cpd 582; Cpd 595; Cpd 640; Cpd 651; Cpd 653; Cpd 664 and Cpd 669 (from I-030).

### Synthesis of N-(4-cyano-2-fluorophenyl)-5-(4-fluorothiophene-3-yl)-1H-pyrrole-3-sulfonamide (Cpd 594)

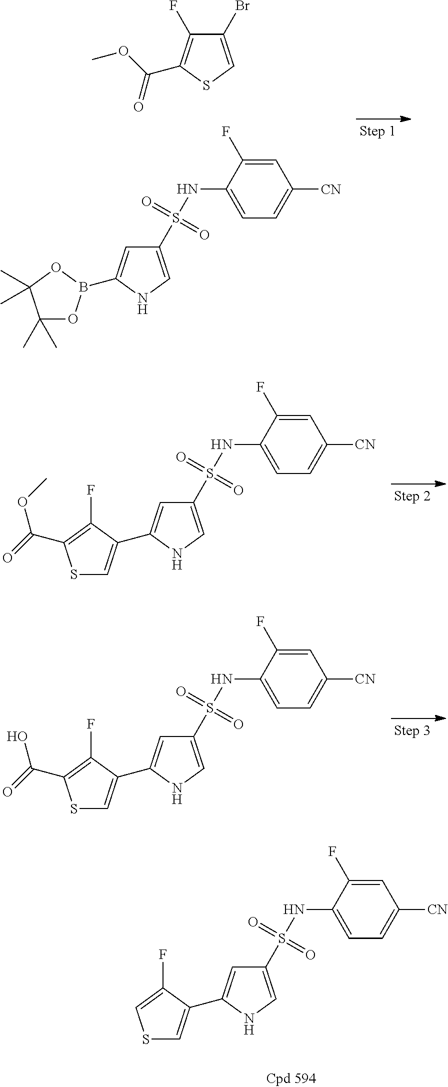 [SMILES: COC(=O)c1scc(Br)c1F ; CC1(C)OB(c2cc(S(=O)(=O)Nc3ccc(C#N)cc3F)c[nH]2)OC1(C)C ; COC(=O)c1scc(-c2cc(S(=O)(=O)Nc3ccc(C#N)cc3F)c[nH]2)c1F ; N#Cc1ccc(NS(=O)(=O)c2c[nH]c(-c3csc(C(=O)O)c3F)c2)c(F)c1 ; N#Cc1ccc(NS(=O)(=O)c2c[nH]c(-c3cscc3F)c2)c(F)c1]

Step 1: To a stirred solution of methyl 4-bromo-3-fluorothiophene-2-carboxylate (140 mg, 0.588 mmol) in 1,4-dioxane (5.0 mL) were added N-(4-cyano-2-fluorophenyl)-5-(4,4,5,5-tetramethyl-1,3,2-dioxaborolan-2-yl)-1H-pyrrole-3-sulfonamide (345.35 mg, 0.883 mmol) and Na₂CO₃ (187.09 mg, 1.765 mmol) in water (0.5 mL). The resulting mixture was degassed under Argon for 15 minutes. Pd(PPh₃)₄ (68 mg, 0.059 mmol) was added and the reaction mixture was heated at 80° C. for 16 hours. Reaction mixture was filtered through a small bed of celite and diluted with ethyl acetate and water. Layers were separated and organic layer was dried over anhydrous Na₂SO₄, filtered and concentrated under reduced pressure. Crude thus obtained was purified by FCC on silica gel using a gradient of EtOAc (10-60%) in hexane to afford 200 mg of crude 4-(4-(N-(4-cyano-2-fluorophenyl)sulfamoyl)-1H-pyrrol-2-yl)-3-fluorothiophene-2-carboxylate that was used in subsequent steps without further purification. LCMS (ES−, m/z) [M−H]⁻=422.3.

Step 2: To a stirred solution of 4-(4-(N-(4-cyano-2-fluorophenyl)sulfamoyl)-1H-pyrrol-2-yl)-3-fluorothiophene-2-carboxylate (180.0 mg, 0.426 mmol) in THF/water (4:1, 5.0 mL) was added LiOH·H₂O (89.274 mg, 2.128 mmol) at 0° C. After addition, the reaction mixture was stirred at RT for 16 hours. Then reaction mass was diluted with water and extracted with EtOAc. Aqueous phase was acidified with 2N HCl (pH˜2.0) and extracted with EtOAc. Organic phases were washed with brine, dried over anhydrous Na₂SO₄, filtered and concentrated under reduced pressure to afford 90 mg (52%) of 4-(4-(N-(4-cyano-2-fluorophenyl)sulfamoyl)-1H-pyrrol-2-yl)-3-fluorothiophene-2-carboxylic acid. ¹H NMR (400 MHz, DMSO-d6): δ ppm 13.42 (br, 1H), 12.27 (s, 1H), 10.54 (s, 1H), 8.00-7.99 (m, 1H), 7.83-7.80 (m, 1H), 7.62-7.57 (m, 3H), 6.65 (s, 1H).

Step 3: To a stirred solution of 4-(4-(N-(4-cyano-2-fluorophenyl)sulfamoyl)-1H-pyrrol-2-yl)-3-fluorothiophene-2-carboxylic acid (150.0 mg, 0.366 mmol) in DMSO (1.0 mL) were added AcOH (0.002 mL, 0.037 mmol) and Silver carbonate (20.207 mg, 0.073 mmol). The resulting RM was heated at 120° C. for 2 hours. Upon completion, RM was diluted with ice-cold water and extracted with EtOAc for several times. The organic part was then dried over anhydrous Na₂SO₄, filtered and concentrated under reduced pressure. Crude thus obtained was purified by FCC on silica gel using a gradient of EtOAc (5-50%) in hexane to afford 80 mg (60%) of N-(4-cyano-2-fluorophenyl)-5-(4-fluorothiophene-3-yl)-1H-pyrrole-3-sulfonamide (Cpd 594).

The following compounds were prepared in a similar manner (use of appropriate reagents and purification methods (including chiral HPLC or chiral SFC) known to the person skilled in the art) as described for Cpd 594: Cpd 602.

### Synthesis of 5-cyclopropyl-N-(2,4,5-trifluorophenyl)-1H-pyrrole-3-sulfonamide (Cpd 213) from I-005

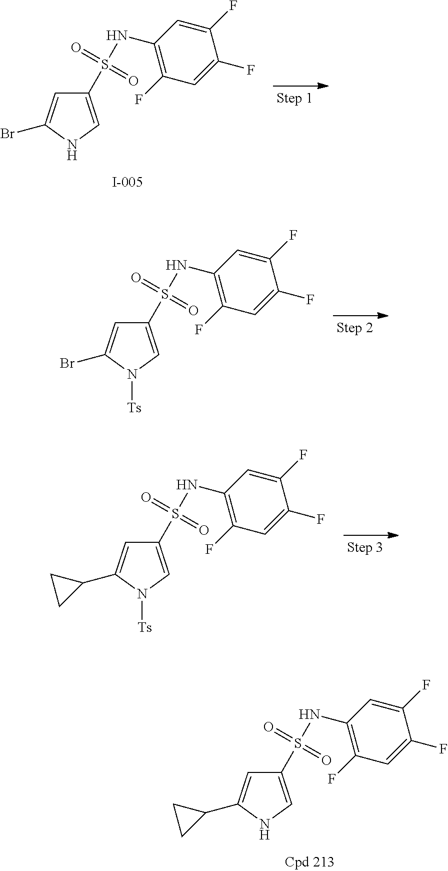 [SMILES: O=S(=O)(Nc1cc(F)c(F)cc1F)c1c[nH]c(Br)c1 ; Cc1ccc(S(=O)(=O)n2cc(S(=O)(=O)Nc3cc(F)c(F)cc3F)cc2Br)cc1 ; Cc1ccc(S(=O)(=O)n2cc(S(=O)(=O)Nc3cc(F)c(F)cc3F)cc2C2CC2)cc1 ; O=S(=O)(Nc1cc(F)c(F)cc1F)c1c[nH]c(C2CC2)c1]

Step 1: To a solution of 5-bromo-N-(2,4,5-trifluorophenyl)-1H-pyrrole-3-sulfonamide (I-005) (250 mg, 0.71 mmol) in THF (5 mL) was added NaH (60% in mineral oil) (62 mg, 1.55 mmol) at 0° C. After 30 min. at RT, TsCl (673 mg, 3.53 mmol) was added. The RM was stirred at RT for 2 h. The RM was partitioned between EtOAc and a sat. NH₄Cl solution. Organic layer was dried over Na₂SO₄, filtrated, and concentrated under reduced pressure. The residue was purified by FCC over silica gel using a gradient of EtOAc (0 to 15%) to afford 200 mg (56%) of 5-bromo-1-tosyl-N-(2,4,5-trifluorophenyl)-1H-pyrrole-3-sulfonamide.

Step 2: To a solution of 5-bromo-1-tosyl-N-(2,4,5-trifluorophenyl)-1H-pyrrole-3-sulfonamide (500 mg, 0.982 mmol) in toluene (20 ml) were added cyclopropylboronic acid (211 mg, 0.98 mmol), K₃PO₄ (521 mg, 2.5 mmol) and tricyclohexylphosphine (28 mg, 0.098 mmol). The RM was degassed with argon before the addition of Pd(OAc)₂ (11 mg, 0.049 mmol). The RM was heated at 110° C. for 16 h. The RM was concentrated under reduced pressure. Water was added to the residue. The aqueous phase was extracted thrice with EtOAc. The organic layers were combined, washed with brine, dried over Na₂SO₄, filtrated, concentrated under reduced pressure. The residue was purified by FCC over silica gel using a gradient of EtOAc (0 to 20%) in hexane to afford 50 mg (11%) of 5-cyclopropyl-1-tosyl-N-(2,4,5-trifluorophenyl)-1H-pyrrole-3-sulfonamide.

Step 3: To a solution of 5-cyclopropyl-1-tosyl-N-(2,4,5-trifluorophenyl)-1H-pyrrole-3-sulfonamide (50 mg, 0.106 mmol) in a mixture of MeOH (3 mL) and water (0.5 mL) was added NaOH (21 mg, 0.53 mmol) at 0° C. The RM was stirred for 3 h at RT. After completion, the pH of the RM was adjusted to pH˜7 and extracted with DCM. The organic layers were combined, washed with brine, dried over Na₂SO₄, filtered, concentrated under reduced pressure. The residue was purified by preparative HPLC on a YMC Actus Triart C18 (250×20 mm, 5N) column, with a flow rate of 16 mL/min. Mobile phase: A=20 mM NH₄HCO₃ in water, B=MeCN; Gradient Profile: Mobile phase initial composition of 80% A and 20% B, then 75% A and 25% B in 2 min, then to 45% A and 55% B in 22 min., then to 5% A and 95% B in 23 min., held this composition up to 25 min. The purification afforded 10 mg (30%) of 5-cyclopropyl-N-(2,4,5-trifluorophenyl)-1H-pyrrole-3-sulfonamide (Cpd 213).

The following compounds were prepared in a similar manner (use of appropriate reagents and purification methods (including chiral HPLC or chiral SFC) known to the person skilled in the art) as described for Cpd 213: Cpd 236 (from I-006) and Cpd 240 (from I-006).

### Synthesis of N-(4-cyano-2-fluorophenyl)-5-(cyclopropylmethyl)-1H-pyrrole-3-sulfonamide (Cpd 622) from I-006

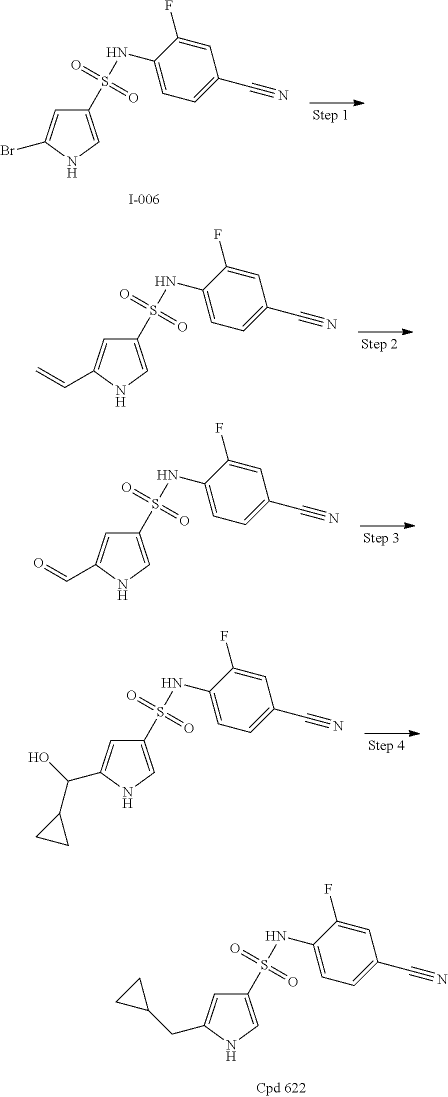 [SMILES: N#Cc1ccc(NS(=O)(=O)c2c[nH]c(Br)c2)c(F)c1 ; C=Cc1cc(S(=O)(=O)Nc2ccc(C#N)cc2F)c[nH]1 ; N#Cc1ccc(NS(=O)(=O)c2c[nH]c(C=O)c2)c(F)c1 ; N#Cc1ccc(NS(=O)(=O)c2c[nH]c(C(O)C3CC3)c2)c(F)c1 ; N#Cc1ccc(NS(=O)(=O)c2c[nH]c(CC3CC3)c2)c(F)c1]

Step 1: A stirred mixture of 5-bromo-N-(4-cyano-2-fluorophenyl)-1H-pyrrole-3-sulfonamide (I 006) (260 mg, 0.755 mmol) in 1,4-dioxane (5.0 mL) was degassed under argon atmosphere for 15 minutes followed by the addition of tributyl(vinyl)stannane (311.42 mg, 0.982 mmol), PPh₃ (9.91 mg, 0.038 mmol) and Pd(PPh₃)₄ (43.65 mg, 0.038 mmol). The RM was heated at 110° C. for 16 hours. After completion, the volatiles were removed by evaporation under reduced pressure. The crude thus obtained was purified by FCC over silica gel using a gradient of EtOAc (0 to 10%) in DCM to afford 150 mg (68%) of N-(4-cyano-2-fluorophenyl)-5-vinyl-1H-pyrrole-3-sulfonamide. ¹H NMR (400 MHz, DMSO-d6): δ ppm 11.91 (s, 1H), 10.47 (s, 1H), 7.80 (d, 1H), 7.58-7.54 (m, 2H), 7.42 (s, 1H), 6.51-6.44 (m, 1H), 6.39 (s, 1H), 5.61-5.56 (m, 1H), 5.10-5.07 (m, 1H).

Step 2: A mixture of N-(4-cyano-2-fluorophenyl)-5-vinyl-1H-pyrrole-3-sulfonamide (150 mg, 0.515 mmol) and OsO₄ (2.62 mg, 0.01 mmol) in THF/water (3:1, 8.0 mL) was stirred at RT for 20 minutes followed by the addition of sodium periodate (280 mg, 1.309 mmol). The reaction mixture was stirred at RT for 4 hours. The reaction was quenched by addition of crushed ice. The solid formed was filtered and triturated with pentane and diethyl ether to afford 130 mg (86%) of N-(4-cyano-2-fluorophenyl)-5-formyl-1H-pyrrole-3-sulfonamide. ¹H NMR (400 MHz, DMSO-d6): δ ppm 12.94 (s, 1H), 10.68 (s, 1H), 9.54 (s, 1H), 7.82 (d, 1H), 7.72 (s, 1H), 7.62-7.55 (m, 2H), 7.27 (s, 1H).

Step 3: To a stirred mixture of N-(4-cyano-2-fluorophenyl)-5-formyl-1H-pyrrole-3-sulfonamide (130 mg, 0.444 mmol) in dry THF (10.0 mL), Cyclopropyl magnesium bromide (0.5 M, 0.976 mL, 0.488 mmol) was added drop wise at −78° C. under N₂ atmosphere. After complete addition the RM was stirred at 0° C. for 4 hours. After completion, RM was quenched with NH₄Cl solution and extracted with EtOAc. Organic phase was separated, dried over anhydrous Na₂SO₄, filtered and concentrated. The residue was purified by FCC over silica gel using a gradient of MeOH (0 to 2%) in DCM to afford 110 mg (74%) of N-(4-cyano-2-fluorophenyl)-5-(cyclopropyl(hydroxy)methyl)-1H-pyrrole-3-sulfonamide. LCMS (ES−, m/z) [M−H]⁻=334.1. ¹H NMR (400 MHz, DMSO-d6): δ ppm 11.51 (s, 1H), 10.40 (s, 1H), 7.79 (d, 1H), 7.59-7.56 (m, 2H), 7.27 (s, 1H), 6.20 (s, 1H), 5.24-5.23 (m, 1H), 3.88-3.86 (m, 1H), 1.07-1.05 (m, 1H), 0.42-0.38 (m, 2H), 0.32-0.22 (m, 2H).

Step 4: To a mixture of N-(4-cyano-2-fluorophenyl)-5-(cyclopropyl(hydroxy)methyl)-1H-pyrrole-3-sulfonamide (50.0 mg, 0.149 mmol) in DCE (2.0 mL) at 0° C. was added TFA (0.115 mL, 1.492 mmol) and triethylsilane (0.026 mL, 0.164 mmol). The reaction mixture was stirred at 0° C. for 1 hour. After completion, reaction mixture was diluted with EtOAc and quenched with aq. sodium bicarbonate solution. Organic phase was separated, dried over anhydrous Na₂SO₄, filtered and concentrated. Crude thus obtained was purified by R^{p} preparative HPLC on a YMC-Actus Triart C18 column (20×250 mm, 5 μm) operating at ambient temperature and flow rate of 16 mL/min; Mobile phase A: 20 mM NH₄HCO₃ in water; Mobile phase B: MeCN; Gradient profile: mobile phase initial composition of 20% B, then 35% B in 5 min., then to 65% B in 30 min., then to 95% B in 31 min., held this composition up to 33 min. for column washing, then returned to initial composition in 34 min. and held till 36 mins. The purification afforded to afford 8 mg (17%) of N-(4-cyano-2-fluorophenyl)-5-(cyclopropylmethyl)-1H-pyrrole-3-sulfonamide (Cpd 622).

### Synthesis of 5-cyclobutyl-N-(2,5-difluoro-4-(trifluoromethyl)phenyl)-1H-pyrrole-3-sulfonamide (Cpd 430)

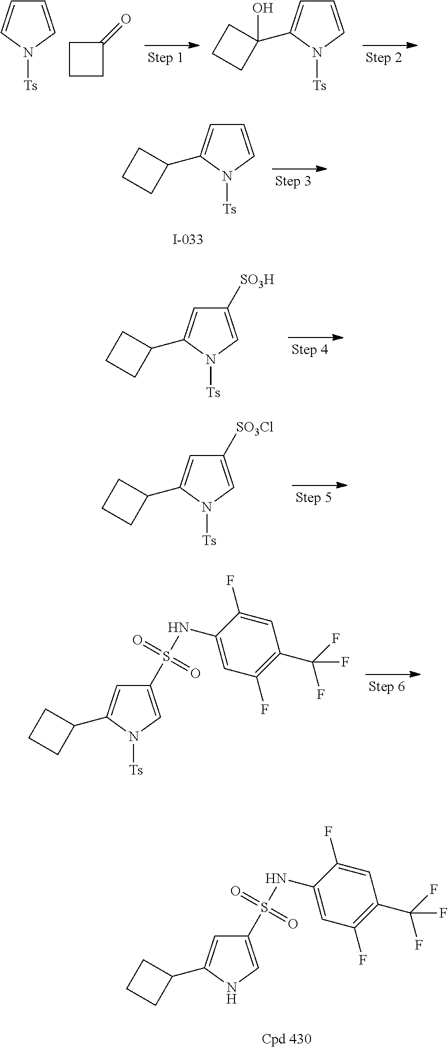

Step 1: To the stirred mixture of 1-tosyl-1H-pyrrole (10.0 g, 45.194 mmol) in dry THF (30.0 mL), t-BuLi (1.7 M, 29.24 mL, 49.713 mmol) was added drop wise at −78° C. and the reaction mixture was stirred for 20 minutes at same temperature. After formation of des-bromo as evidenced from TLC, a solution of cyclobutanone (3.168 g, 45.194 mmol) in THF (2.0 mL) was added drop wise at −78° C. under inert atmosphere. Reaction mixture was stirred for 4 hours at same temperature. After completion, reaction mixture was quenched with saturated aqueous NH₄Cl solution and extracted with ethyl acetate. Organic phase was evaporated under reduced pressure and crude thus obtained was purified by FCC over silica gel using a gradient of EtOAc (0 to 2%) in hexane to afford 3.2 g (24%) of 1-(1-tosyl-1H-pyrrol-2-yl) cyclobutan-1-ol. ¹H NMR (400 MHz, DMSO-d6): δ ppm 7.82 (d, 2H), 7.44-7.31 (m, 3H), 6.33-6.20 (m, 2H), 5.30 (s, 1H), 2.66-2.63 (m, 1H), 2.50-2.46 (m, 2H), 2.35 (s, 3H), 2.27-2.13 (m, 2H), 1.75-1.73 (m, 1H), 1.49-1.42 (m, 1H).

Step 2: To a stirred solution of 1-(1-tosyl-1H-pyrrol-2-yl) cyclobutan-1-ol in DCM (10.0 mL) was added triethylsilane (3.07 mL, 19.238 mmol) and TFA (13.14 mL, 171.768 mmol) and the reaction mixture was stirred in a sealed vial at 90° C. for 2 hours. Upon completion, the reaction mixture was evaporated under reduced pressure, diluted with EtOAc and washed with saturated aq. NaHCO₃solution and brine solution. The organic phase was dried over anhydrous Na₂SO₄, filtered and evaporated under reduced pressure. Crude thus obtained was purified by FCC over silica gel using a gradient of EtOAc (20 to 30%) in hexane to afford 3 g (63%) of 2-cyclobutyl-1-tosyl-1H-pyrrole. ¹H NMR (400 MHz, DMSO-d6): δ ppm 7.68 (d, 2H), 7.43 (d, 2H), 7.31 (brs, 1H), 6.28-6.27 (m, 1H), 6.22 (br s, 1H), 3.63-3.59 (m, 1H), 2.37 (s, 3H), 2.14-2.12 (m, 2H), 1.92-1.81 (m, 3H), 1.71-1.69 (m, 1H).

Step 3: To a stirred solution of 2-cyclobutyl-1-tosyl-1H-pyrrole (1.0 g, 3.631 mmol) in MeCN (10.0 mL) was added Chlorosulfonic acid (1.2 mL, 18.157 mmol) drop wise at 0° C. RM was stirred at 0° C. for 1 hour. After completion, the RM was evaporated under reduced pressure and crude thus obtained was diluted with 10% MeOH/DCM. It was neutralized with 10% aq. K₂CO₃ solution. The organic phase was separated, evaporated under reduced pressure to afford 1.2 g of crude 5-cyclobutyl-1-tosyl-1H-pyrrole-3-sulfonic acid (I-033) that was used in subsequent step without further purification. LCMS (ES−, m/z) [M−H]⁻=354.23.

Step 4: A stirred solution of 5-cyclobutyl-1-tosyl-1H-pyrrole-3-sulfonic acid (I-033) (1.2 g, 3.376 mmol) in MeCN (10.0 mL) was cooled to 0° C. POCl₃ (1.6 mL, 6.881 mmol) was then added drop wise and the reaction mixture was heated at 80° C. for 3 hours. After completion, RM was evaporated to remove the solvent, quenched with ice and extracted with 10% MeOH/DCM. Organic part was dried over anhydrous Na₂SO₄, filtered and evaporated under reduced pressure. Crude thus obtained was purified by FCC over silica gel using a gradient of MeOH (0 to 5%) in DCM to afford 1 g of 5-cyclobutyl-1-tosyl-1H-pyrrole-3-sulfonyl chloride (I-035). LCMS (ES−, m/z) [M−H]⁻=436.2 (quenched with N-Methyl piperazine).

Step 5: In a 10 ml screwed cap vial 5-cyclobutyl-1-tosyl-1H-pyrrole-3-sulfonyl chloride (I-035) (300 mg, 0.802 mmol), 2,5-difluoro-4-(trifluoromethyl)aniline (237.24 mg, 1.204 mmol) were mixed with MeCN (5.0 mL). Then pyridine (0.323 mL, 4.012 mmol) was added and reaction mixture was heated at 80° C. for 12 hours. After completion, reaction mixture was evaporated and crude thus obtained was purified by FCC over silica gel using a gradient of DCM (0 to 70%) in hexane to afford 250 mg (58%) of 5-cyclobutyl-N-(2,5-difluoro-4-(trifluoromethyl)phenyl)-1-tosyl-1H-pyrrole-3-sulfonamide. LCMS (ES−, m/z) [M−H]⁻=532.8.

Step 6: To a stirred mixture of 5-cyclobutyl-N-(2,5-difluoro-4-(trifluoromethyl)phenyl)-1-tosyl-1H-pyrrole-3-sulfonamide (250 mg, 0.468 mmol) in MeOH/Water (2:1, 6.0 mL), aq. KOH solution (5M, 0.6 mL) was added and heated to reflux for 30 minutes. After completion, all the volatiles were removed. Crude thus obtained was purified first by FCC over silica gel eluting with 2% MeOH in DCM and second by R^{p} preparative HPLC on a YMC-Actius 018 column (20×250 mm, 5 μm) operating at RT with a flow rate of 16 mL/min; Mobile Phase A: 20 mM NH₄HCO₃ in water; Mobile Phase B: MeOH; Gradient profile: 40% B to 60% B in 5 min, then 85% B in 25 min and to 95% in 1 minute, held for 2 min for column washing, then returned to initial composition in 1 min and held for 2 min. The purification afforded 140 mg (79%) of 5-cyclobutyl-N-(2,5-difluoro-4-(trifluoromethyl)phenyl)-1H-pyrrole-3-sulfonamide (Cpd 430).

The following compounds were prepared in a similar manner (use of appropriate reagents and purification methods (including chiral HPLC or chiral SFC) known to the person skilled in the art) as described for Cpd 430: Cpd 550 and Cpd 571.

### Synthesis of N-(4-bromo-2,5-difluorophenyl)-5-cyclobutyl-1H-pyrrole-3-sulfonamide (Cpd 551)

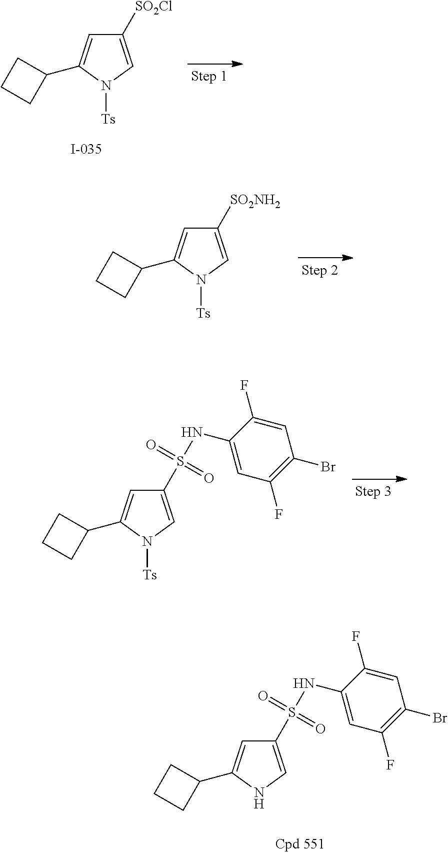 [SMILES: Cc1ccc(S(=O)(=O)n2cc(S(=O)(=O)Cl)cc2C2CCC2)cc1 ; Cc1ccc(S(=O)(=O)n2cc(S(N)(=O)=O)cc2C2CCC2)cc1 ; Cc1ccc(S(=O)(=O)n2cc(S(=O)(=O)Nc3cc(F)c(Br)cc3F)cc2C2CCC2)cc1 ; O=S(=O)(Nc1cc(F)c(Br)cc1F)c1c[nH]c(C2CCC2)c1]

Step 1: To the stirred mixture of 5-cyclobutyl-1-tosyl-1H-pyrrole-3-sulfonyl chloride (I-035) (250 mg, 0.67 mmol) in dry THF (10.0 mL), aq. NH₃ (4.0 mL) was added at 0° C. and stirred for 1 hour at RT. After completion, RM was poured in ice cooled water and extracted with EtOAc. Organic phase was separated, dried over anhydrous Na₂SO₄, filtered and concentrated under reduced pressure to afford 195 mg (82%) of crude 5-cyclobutyl-1-tosyl-1H-pyrrole-3-sulfonamide that was used in the subsequent step without purification. LCMS (ES−, m/z) [M−H]⁻=353.2.

Step 2: To a stirred degassed mixture of 5-cyclobutyl-1-tosyl-1H-pyrrole-3-sulfonamide (250 mg, 0.705 mmol) in dry MeCN (5.0 mL) were added 1,4-dibromo-2,5-difluorobenzene (761.34 mg, 2.821 mmol), K₂CO₃ (243.7 mg, 1.763 mmol), CuI (45.7 mg, 0.24 mmol) and trans-N,N-dimethylcyclohexane 1,2 diamine (80.3 mg, 0.8 mmol). The RM was stirred at 80° C. for 16 hours. After completion, the RM was passed through celite bed and filtrate was concentrated under reduced pressure. Crude thus obtained was purified by FCC over silica gel using a gradient of MeOH (0 to 1%) in DCM to afford 160 mg (42%) of N-(4-bromo-2,5-difluorophenyl)-5-cyclobutyl-1-tosyl-1H-pyrrole-3-sulfonamide. LCMS (ES−, m/z) [M−H]⁻=543.2, 545.2.

Step 3: To the stirred mixture of N-(4-bromo-2,5-difluorophenyl)-5-cyclobutyl-1-tosyl-1H-pyrrole-3-sulfonamide (200 mg, 0.367 mmol) in MeOH/Water (2:1, 6.0 mL), aqueous KOH solution (5M, 0.6 mL) was added and the mixture was heated to reflux for 30 minutes. After completion, all the volatiles were removed and crude thus obtained was purified by FCC over silica gel using a gradient of MeOH (0 to 2%) in DCM to afford 85 mg (59%) of N-(4-bromo-2,5-difluorophenyl)-5-cyclobutyl-1H-pyrrole-3-sulfonamide (Cpd 551).

The following compounds were prepared in a similar manner (use of appropriate reagents and purification methods (including chiral HPLC or chiral SFC) known to the person skilled in the art) as described for Cpd 551: Cpd 549.

### Synthesis of N-(4-cyano-2-fluorophenyl)-5-(pyrimidin-2-yl)-1H-pyrrole-3-sulfonamide (Cpd 094)

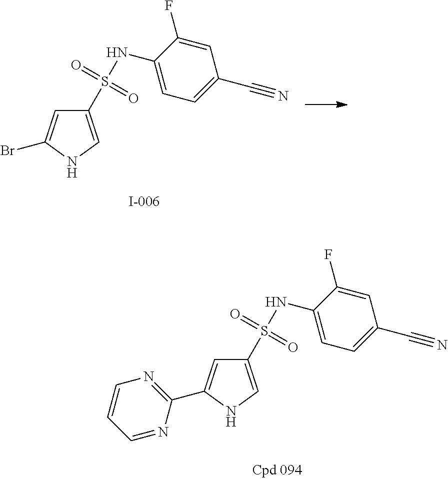 [SMILES: N#Cc1ccc(NS(=O)(=O)c2c[nH]c(Br)c2)c(F)c1 ; N#Cc1ccc(NS(=O)(=O)c2c[nH]c(-c3ncccn3)c2)c(F)c1]

To a solution of 5-bromo-N-(4-cyano-2-fluorophenyl)-1H-pyrrole-3-sulfonamide (I-006) (200 mg, 0.58 mmol) in dry DMF (5 mL) was added 2-(tributylstannyl)pyrimidine (428 mg, 1.16 mmol), Pd(PPh₃)₄ (67 mg, 0.06 mmol) at RT. The RM was stirred overnight at 130° C. under N₂. The volatiles were removed under reduced pressure. The residue was purified by FCC over silica gel using a gradient of EtOAc (10 to 20%) in PE. The residue was further purified by preparative HPLC on a YMC-Actus Triart C18 Column (20×250 mm, 5 μm); Mobile Phase A: Water (0.1% FA), Mobile Phase B: MeCN; Flow rate: 25 mL/min; Gradient: 28% to 57% of B in 9 min. The purification afforded 32 mg (16%) of afford N-(4-cyano-2-fluorophenyl)-5-(pyrimidin-2-yl)-1H-pyrrole-3-sulfonamide (Cpd 094).

### Synthesis of N-(4-cyano-2-fluorophenyl)-5-cyclobutyl-1H-pyrrole-3-sulfonamide (Cpd 098) from I-006

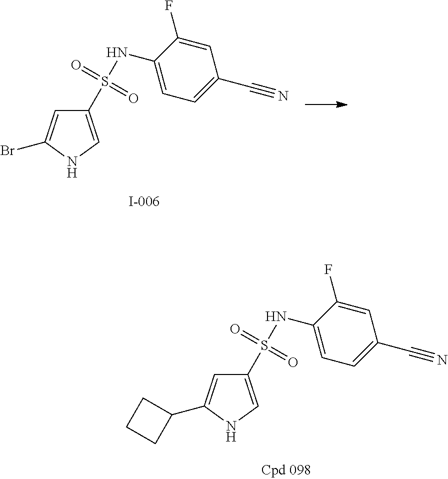 [SMILES: N#Cc1ccc(NS(=O)(=O)c2c[nH]c(Br)c2)c(F)c1 ; N#Cc1ccc(NS(=O)(=O)c2c[nH]c(C3CCC3)c2)c(F)c1]

To a vial (30 mL) equipped with a stirring bar were added [Ir{dF(CF₃)ppy}₂(dtbpy)]PF₆ (6.5 mg, 0.006 mmol), 5-bromo-N-(4-cyano-2-fluorophenyl)-1H-pyrrole-3-sulfonamide (I-006) (200 mg, 0.58 mmol), bromocyclobutane (157 mg, 1.16 mmol), tris(trimethylsilyl)silane (144 mg, 0.58 mmol), and Na₂CO₃ (123 mg, 1.16 mmol). The vial was sealed and degassed with N₂. DME (20 mL) was added. A degassed solution of dichloro(dimethoxyethane)nickel (6.4 mg, 0.029 mmol) and 4-tert-butyl-2-(4-tert-butylpyridin-2-yl)pyridine (7.8 mg, 0.029 mmol) in DME (5 mL) was added to the RM. The RM was degassed again with N₂ during 10 min. The RM was stirred and irradiated with a 34 W blue LED lamp (7 cm away, with cooling fan to keep the reaction temperature at 25° C.) overnight. The RM was concentrated under reduced pressure. The residue was purified by FCC over silica gel using EtOAc (100%) as eluent. The residue was further purified by preparative HPLC on a SunFire Prep 018 OBD Column (19×150 mm, 5 μm); Mobile Phase A: Water (0.1% FA), Mobile Phase B: MeCN; Flow rate: 25 mL/min; Gradient: 34% to 50% of B in 9 min. The purification afforded 23 mg (12%) of N-(4-cyano-2-fluorophenyl)-5-cyclobutyl-1H-pyrrole-3-sulfonamide (Cpd 098).

### Synthesis of N-(4-cyano-2-fluorophenyl)-5-(guinolin-8-yl)-1H-pyrrole-3-sulfonamide (Cpd 380)

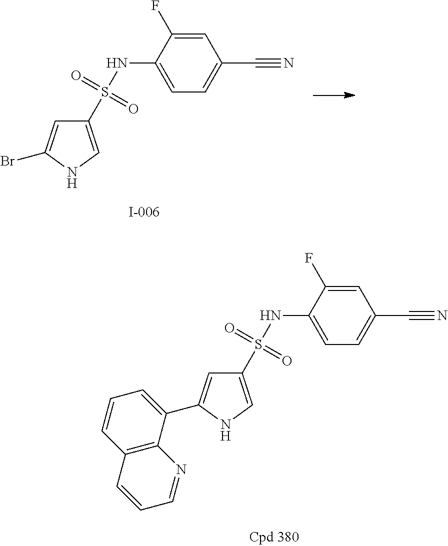 [SMILES: N#Cc1ccc(NS(=O)(=O)c2c[nH]c(Br)c2)c(F)c1 ; N#Cc1ccc(NS(=O)(=O)c2c[nH]c(-c3cccc4cccnc34)c2)c(F)c1]

To a stirred degassed solution of 5-bromo-N-(4-cyano-2-fluorophenyl)-1H-pyrrole-3-sulfonamide (I-006) (150 mg, 0.436 mmol) in tert-Amyl alcohol (5.0 ml) was added 8-Quinolineboronic acid (114 mg, 0.658 mmol). A solution of K₂CO₃ (181.58 mg, 1.316 mmol) in water (0.5 ml) was added to the reaction mixture and resulting mixture was degassed with argon followed by the addition of Pd(amphos)Cl₂ (31 mg, 0.044 mmol). The resulting reaction mixture was then stirred at 80° C. for 16 h. Reaction mixture was monitored by LCMS. Solvent was evaporated under reduced pressure. The residue was purified by FCC on silica gel using a gradient of EtOAc (0-10%) in DCM. The residue was further purified by preparative HPLC on a YMC-Actus Triart C18 column (20×250 mm, 5 μm) operating at ambient temperature and flow rate of 16 mL/min; Mobile phase A: 20 mM NH₄HCO₃ in water; Mobile phase B: MeCN; Gradient profile: mobile phase initial composition of 70% A and 30% B, then to 50% A and 50% B in 5 min., then to 25% A and 75% B in 30 min., then to 5% A and 95% B in 31 min., held this composition up to 33 min. for column washing, then returned to initial composition in 34 min. and held till 36 mins. The purification afforded 17 mg (11%) of N-(4-cyano-2-fluorophenyl)-4-(4-fluorophenyl)-1H-pyrrole-3-sulfonamide (Cpd 380).

The following compounds were prepared in a similar manner (use of appropriate reagents and purification methods (including chiral HPLC or chiral SFC) known to the person skilled in the art) as described for Cpd 380: Cpd 382.

### Synthesis of N-(4-bromo-2,5-difluorophenyl)-5-(thiophen-2-yl)-1H-pyrrole-3-sulfonamide (Cpd 478)

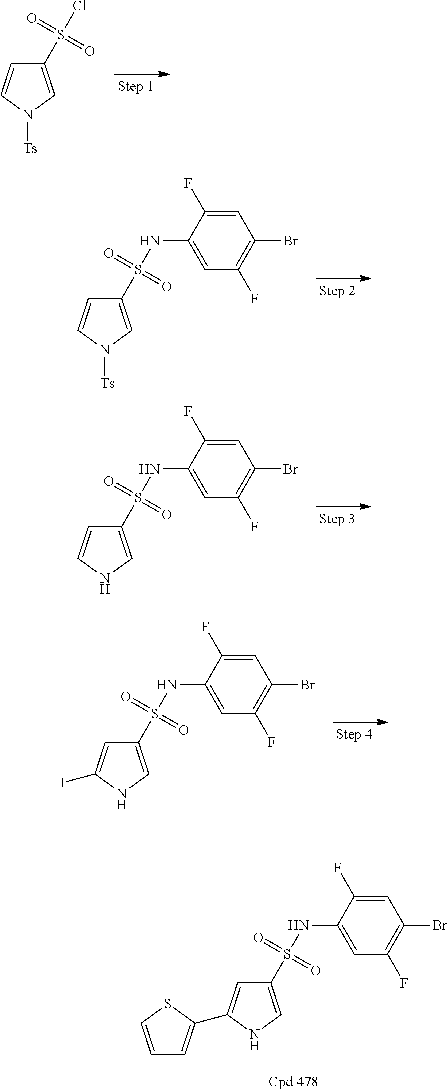 [SMILES: Cc1ccc(S(=O)(=O)n2ccc(S(=O)(=O)Cl)c2)cc1 ; Cc1ccc(S(=O)(=O)n2ccc(S(=O)(=O)Nc3cc(F)c(Br)cc3F)c2)cc1 ; O=S(=O)(Nc1cc(F)c(Br)cc1F)c1cc[nH]c1 ; O=S(=O)(Nc1cc(F)c(Br)cc1F)c1c[nH]c(I)c1 ; O=S(=O)(Nc1cc(F)c(Br)cc1F)c1c[nH]c(-c2cccs2)c1]

Step 1: A mixture of 1-(4-methyl benzenesulfonyl) pyrrole-3-sulfonyl chloride (13.2 g, 41.28 mmol) and 4-bromo-2,5-difluoroaniline (12.88 g, 61.92 mmol) in pyridine (200 mL) was stirred for 12 h at 80° C. under N₂ atmosphere. The mixture was allowed to cool down to RT. The RM was concentrated under reduced pressure. The residue was purified by FCC on silica gel eluting with EtOAc/PE (1:3) to afford 7.1 g (35%) of N-(4-bromo-2,5-difluorophenyl)-1-(4-methylbenzenesulfonyl) pyrrole-3-sulfonamide. ¹H NMR (400 MHz, CHCl₃) δ 7.50-7.33 (m, 5H), 7.16 (d, J=8.2 Hz, 2H), 6.48 (dd, J=1.8, 0.8 Hz, 1H), 6.28 (m, 1H), 6.19 (dd, J=3.3, 1.8 Hz, 1H), 2.36 (s, 3H).

Step 2: A mixture of N-(4-bromo-2,5-difluorophenyl)-1-(4-methyl benzenesulfonyl) pyrrole-3-sulfonamide (7.1 g, 14.45 mmol) and LiOH (1.73 g, 72.26 mmol) in MeOH (40 mL) and water (20 mL) was stirred for 1 h at RT under nitrogen atmosphere. The resulting mixture was concentrated under reduced pressure. The mixture was acidified to pH 7 with aqueous HCl. The aqueous layer was extracted with EtOAc (3×500 mL), dried over anhydrous Na₂SO₄, filtered and concentrated under reduced pressure. The residue was purified by FCC on silica gel eluting with EtOAc/PE (1:3) to afford 3.9 g (80%) of N-(4-bromo-2,5-difluorophenyl)-1H-pyrrole-3-sulfonamide.

Step 3: To a stirred solution of N-(4-bromo-2,5-difluorophenyl)-1H-pyrrole-3-sulfonamide (3.9 g, 11.57 mmol) in DMF (150 mL) was added NIS (2.6 g, 11.57 mmol) dropwise at −50° C. under argon atmosphere. The resulting mixture was stirred for 16 h at RT under nitrogen atmosphere. The resulting mixture was concentrated under reduced pressure. The residue was purified by FCC on silica gel eluting with EtOAc/PE (1:2) to afford 650 mg (12%) of N-(4-bromo-2,5-difluorophenyl)-5-iodo-1H-pyrrole-3-sulfonamide (650 mg, 12%).

Step 4: To a stirred mixture of N-(4-bromo-2,5-difluorophenyl)-5-iodo-1H-pyrrole-3-sulfonamide (800 mg, 1.73 mmol) and thiophen-2-ylboronic acid (221 mg, 1.73 mmol) in dioxane (10 mL) and water (1 mL) were added CsF (525 mg, 3.46 mmol) and Pd(dppf)Cl₂ (127 mg, 0.173 mmol) in one portion at RT under N₂ atmosphere. The resulting mixture was stirred for 3 h at 100° C. under nitrogen atmosphere. The mixture was allowed to cool down to RT and concentrated under vacuum. The crude product was purified by preparative HPLC on a Sunfire prep C18 column (30×150 mm, 5 μm) at a flow rate of 60 mL/min; Mobile Phase A: Water (0.1% FA); Mobile Phase B: MeCN; Gradient profile: 45% B to 65% B in 8 min, 65% B; to afford 120 mg (16%) of N-(4-bromo-2,5-difluorophenyl)-5-(thiophen-2-yl)-1H-pyrrole-3-sulfonamide (Cpd 478).

The following compounds were prepared in a similar manner (use of appropriate reagents and purification methods (including chiral HPLC or chiral SFC) known to the person skilled in the art) as described for Cpd 478: Cpd 477, Cpd 479, Cpd 541 and Cpd 561.

### Synthesis of N-(5-ethynyl-3-fluoro-2-pyridyl)-5-phenyl-1H-pyrrole-3-sulfonamide (Cpd 216) from Cpd 168

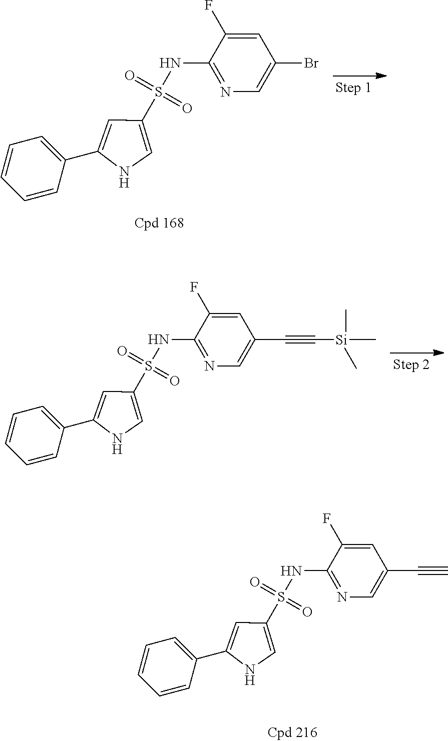 [SMILES: O=S(=O)(Nc1ncc(Br)cc1F)c1c[nH]c(-c2ccccc2)c1 ; C[Si](C)(C)C#Cc1cnc(NS(=O)(=O)c2c[nH]c(-c3ccccc3)c2)c(F)c1 ; C#Cc1cnc(NS(=O)(=O)c2c[nH]c(-c3ccccc3)c2)c(F)c1]

Step 1: To a mixture of N-(5-bromo-3-fluoro-2-pyridyl)-5-phenyl-1H-pyrrole-3-sulfonamide (Cpd 168) (160 mg, 0.4 mmol), trim ethylsilylacetylene (0.07 mL, 0.5 mmol) and dry Et₃N (0.28 mL, 2 mmol) in dry DMF (1.6 mL) were added under inert atmosphere CuI (7.7 mg, 0.04 mmol) and PdCl₂(PPh₃)₂ (28 mg, 0.04 mmol). The RM was stirred at 110° C. for 2 h. After cooling at RT, the RM was concentrated. The residue was taken up with DCM and partitioned with water. An aq. saturated solution of NH₄OH (1 mL) was added to the aqueous layer. Aqueous layer was extracted twice with DCM. The organic layers were combined, washed with brine, dried over MgSO₄, filtered, and concentrated under reduced pressure. The residue was purified by FCC over silica gel using a gradient of EtOAc (0 to 20%) in PE to afford 165 mg (99%) of N-[3-fluoro-5-(2-trimethylsilylethynyl)-2-pyridyl]-5-phenyl-1H-pyrrole-3-sulfonamide.

Step 2: To a mixture of N-[3-fluoro-5-(2-trimethylsilylethynyl)-2-pyridyl]-5-phenyl-1H-pyrrole-3-sulfonamide (167 mg, 0.4 mmol) in dry THF (4 mL) was added under inert atmosphere TBAF (1.2 mL, 1.2 mmol, 1 M in THF). The RM was stirred overnight at RT. The RM was diluted with DCM and partitioned with water. Aqueous layer was extracted twice with DCM. The organic extracts were combined, washed with brine, dried over MgSO₄, filtered, and concentrated under reduced pressure. The residue was purified by FCC over silica gel using a gradient of EtOAc (0 to 5%) in DCM to afford 61 mg (43%) of N-(5-ethynyl-3-fluoro-2-pyridyl)-5-phenyl-1H-pyrrole-3-sulfonamide (Cpd 216).

### Synthesis of N-(5-cyano-3-fluoro-2-pyridyl)-5-phenyl-1H-pyrrole-3-sulfonamide (Cpd 166) from Cpd 168

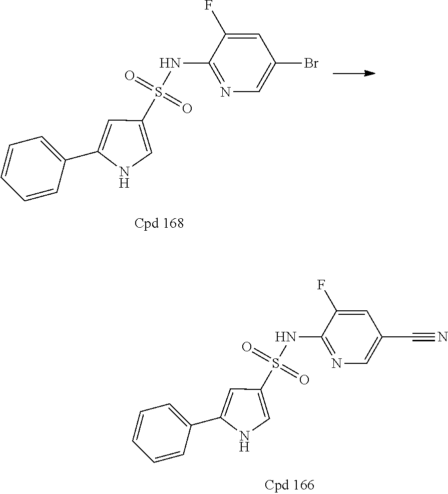 [SMILES: O=S(=O)(Nc1ncc(Br)cc1F)c1c[nH]c(-c2ccccc2)c1 ; N#Cc1cnc(NS(=O)(=O)c2c[nH]c(-c3ccccc3)c2)c(F)c1]

A stirred solution of N-(5-bromo-3-fluoropyridin-2-yl)-5-phenyl-1H-pyrrole-3-sulfonamide (Cpd 168) (150 mg, 0.38 mmol) in DMF (2 mL) was degassed with N₂, followed by the addition of zinc cyanide (267 mg, 2.3 mmol) and Pd(dppf)Cl₂·CH₂Cl₁₋₂ (62 mg, 0.08 mmol). The RM was heated at 130° C. for 16 h. The RM was filtered through celite bed and the filtrate was concentrated under reduced pressure. The residue was purified by preparative HPLC on a YMC Actus Triart C18 (250×20 mm, 5p) column and operating at RT and flow rate of 16 mL/min. Mobile phase: A=20 Mm NH₄HCO₃ in water, B=MeCN; Gradient Profile: Mobile phase initial composition of 80% A and 20% B, then 70% A and 30% B in 3 min, then to 50% A and 50% B in 22 min., then to 5% A and 95% B in 23 min., held this composition up to 26 min. The purification afforded 16 mg (12%) of N-(5-cyano-3-fluoropyridin-2-yl)-5-phenyl-1H-pyrrole-3-sulfonamide (Cpd 166).

### Synthesis of N-[5-(cyanomethyl)-3-methoxypyridin-2-yl]-5-phenyl-1H-pyrrole-3-sulfonamide (Cpd 061) from Cpd 062

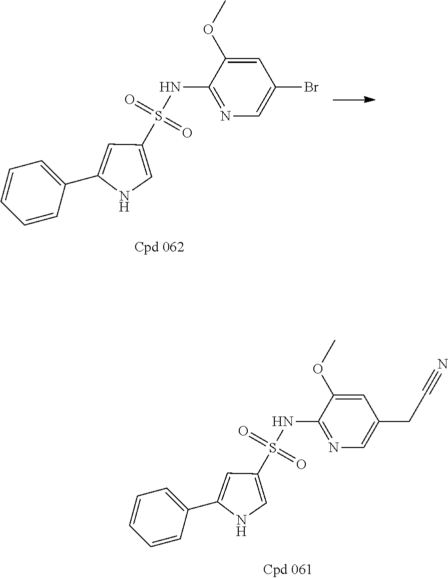 [SMILES: COc1cc(Br)cnc1NS(=O)(=O)c1c[nH]c(-c2ccccc2)c1 ; COc1cc(CC#N)cnc1NS(=O)(=O)c1c[nH]c(-c2ccccc2)c1]

To a solution of N-(5-bromo-3-methoxypyridin-2-yl)-5-phenyl-1H-pyrrole-3-sulfonamide (Cpd 062) (250 mg, 0.61 mmol) and 4-(4,4,5,5-tetramethyl-1,3,2-dioxaborolan-2-yl)-1,2-oxazole (143 mg, 0.74 mmol) in DMSO (7 mL) and H₂O (3 mL) were added KF (107 mg, 1.84 mmol) and Pd(dppf)Cl₂ (22 mg, 0.03 mmol) at RT under N₂. The RM was stirred overnight at 120° C. under N₂. Water (100 mL) was added to the RM and then extracted with EtOAc (3×100 mL). The organic layers were combined, washed with brine (1×100 mL), dried over Na₂SO₄, filtered, and concentrated under reduced pressure. The residue was purified by R^{p} FCC on C18 gel using a gradient of MeCN (50 to 70%) in water with 0.1% FA. The residue was further purified by preparative HPLC on a XBridge Prep C18 OBD Column (19×150 mm, 5 μm); Mobile Phase A: Water (10 mM of NH₄HCO₃), Mobile Phase B: MeCN; Flow rate: 25 mL/min; Gradient: 2% to 33% of B in 2 min. The purification afforded 51.6 mg (23%) of N-[5-(cyanomethyl)-3-methoxypyridin-2-yl]-5-phenyl-1H-pyrrole-3-sulfonamide (Cpd 061).

### Synthesis of N-(2,5-difluoro-4-(trifluoromethyl)phenyl)-5-(3-oxocyclopentyl)-1H-pyrrole-3-sulfonamide (Cpd 520) from N-(2,5-difluoro-4-(trifluoromethyl)phenyl)-5-(3-oxocyclopent-1-en-1-yl)-1H-pyrrole-3-sulfonamide (Cpd 505)

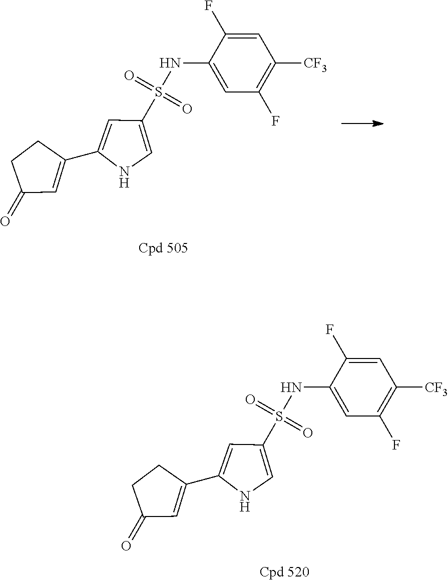 [SMILES: O=C1C=C(c2cc(S(=O)(=O)Nc3cc(F)c(C(F)(F)F)cc3F)c[nH]2)CC1 ; O=C1C=C(c2cc(S(=O)(=O)Nc3cc(F)c(C(F)(F)F)cc3F)c[nH]2)CC1]

To a mixture of N-(2,5-difluoro-4-(trifluoromethyl)phenyl)-5-(3-oxocyclopent-1-en-1-yl)-1H-pyrrole-3-sulfonamide (Cpd 505) (70.0 mg, 0.172 mmol) in MeOH (5.0 mL) was added 10% Pd/C (10 mg) followed by triethylsilane (0.275 mL, 1.723 mmol). The reaction mixture was stirred at RT for 2 h. RM was evaporated under reduced pressure and crude thus obtained was purified by FCC over silica gel using a gradient of EtOAc (0 to 5%) in DCM to afford 30 mg (43%) of N-(2,5-difluoro-4-(trifluoromethyl) phenyl)-5-(3-oxocyclopentyl)-1H-pyrrole-3-sulfonamide (Cpd 520).

### Synthesis of N-(4-cyano-2-fluorophenyl)-2-fluoro-5-phenyl-1H-pyrrole-3-sulfonamide (Cpd 020) and N-(4-cyano-2-fluorophenyl)-4-fluoro-5-phenyl-1H-pyrrole-3-sulfonamide (Cpd 023) from Cpd 002

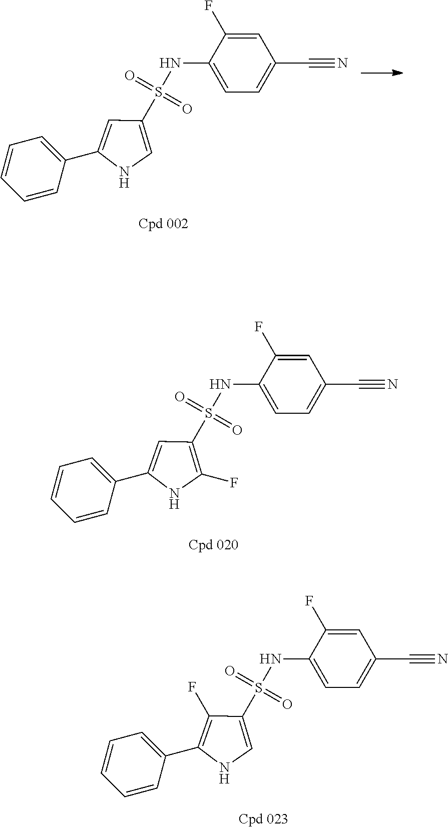 [SMILES: N#Cc1ccc(NS(=O)(=O)c2c[nH]c(-c3ccccc3)c2)c(F)c1 ; N#Cc1ccc(NS(=O)(=O)c2cc(-c3ccccc3)[nH]c2F)c(F)c1 ; N#Cc1ccc(NS(=O)(=O)c2c[nH]c(-c3ccccc3)c2F)c(F)c1]

A mixture of N-(4-cyano-2-fluorophenyl)-5-phenyl-1H-pyrrole-3-sulfonamide (Cpd 002) (750 mg, 2.20 mmol) and NFSI (830 mg, 2.64 mmol) in MeCN (12 mL) was stirred overnight at 120° C. under N₂. The RM was concentrated under reduced pressure. The residue was purified by preparative TLC (Eluent: PE/EtOAc: 4/1). The residue was further purified by preparative HPLC on a XSelect CSH Prep C18 OBD Column (19×150 mm, 5 μm); Mobile Phase A: Water (0.05% FA), Mobile Phase B: MeCN; Flow rate: 25 mL/min; Gradient: 37% to 47% of B in 10 min. The residue was further purified by Prep-TLC (Eluent: PE/EtOAc: 5/1) to afford the pure mixture of Cpd 020 and Cpd 023. The residue was purified by R^{p} FCC on C18 gel using a gradient of MeCN (0 to 100%) in water with 0.1% FA to afford 29 mg (4%) of N-(4-cyano-2-fluorophenyl)-2-fluoro-5-phenyl-1H-pyrrole-3-sulfonamide (Cpd 020) and 3 mg (0.4%) of N-(4-cyano-2-fluorophenyl)-4-fluoro-5-phenyl-1H-pyrrole-3-sulfonamide (Cpd 023).

The following compounds were prepared in a similar manner (use of appropriate reagents and purification methods (including chiral HPLC or chiral SFC) known to the person skilled in the art) as described for Cpd 020 and Cpd 023: Cpd 076 (from Cpd 028) and Cpd 077 (from Cpd 028).

### Synthesis of N-[2,5-difluoro-4-(trifluoromethyl) phenyl]-4-fluoro-5-phenyl-1H-pyrrole-3-sulfonamide (Cpd 626) and N-[2,5-difluoro-4-(trifluoromethyl)phenyl]-2-fluoro-5-phenyl-1H-pyrrole-3-sulfonamide (Cpd 627) from Cpd 071

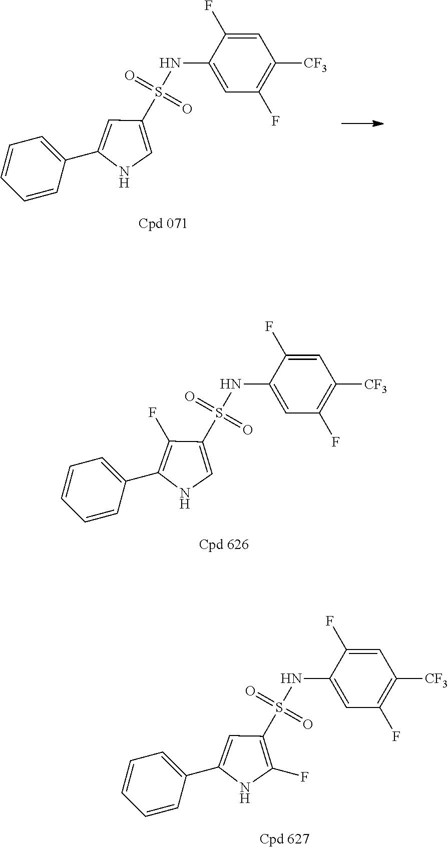 [SMILES: O=S(=O)(Nc1cc(F)c(C(F)(F)F)cc1F)c1c[nH]c(-c2ccccc2)c1 ; O=S(=O)(Nc1cc(F)c(C(F)(F)F)cc1F)c1c[nH]c(-c2ccccc2)c1F ; O=S(=O)(Nc1cc(F)c(C(F)(F)F)cc1F)c1cc(-c2ccccc2)[nH]c1F]

A mixture of N-[2,5-difluoro-4-(trifluoromethyl) phenyl]-5-phenyl-1H-pyrrole-3-sulfonamide (400 mg, 0.994 mmol) and 1-chloromethyl-4-fluoro-1,4-diazoniabicyclo[2.2.2]octane bis(tetrafluoroborate) (352 mg, 0.994 mmol) in EtOAc (5 mL) was stirred for 16 h at 50° C. under nitrogen atmosphere. The mixture was allowed to cool down to RT. The resulting mixture was concentrated under reduced pressure. The crude product was purified by preparative HPLC on a YMC-PACK CN column (30×250 mm, 5 μm) operating at flow rate of 40 mL/min; Mobile Phase A: Hexane (10 mM NH₃-MeOH); Mobile Phase B: IPA; Isocratic: 10% B in 24 min. The purification afforded 70 mg (16%) of N-[2,5-difluoro-4-(trifluoromethyl) phenyl]-4-fluoro-5-phenyl-1H-pyrrole-3-sulfonamide (Cpd 626) and 30 mg (7%) of N-[2,5-difluoro-4-(trifluoromethyl) phenyl]-2-fluoro-5-phenyl-1H-pyrrole-3-sulfonamide (Cpd 627).

The following compounds were prepared in a similar manner (use of appropriate reagents and purification methods (including chiral HPLC or chiral SFC) known to the person skilled in the art) as described for Cpd 626 and Cpd 627: Cpd 632 from Cpd 065).

### Synthesis of N-(4-cyano-2-fluorophenyl)-2-methyl-5-phenyl-1H-pyrrole-3-sulfonamide (Cpd 001)

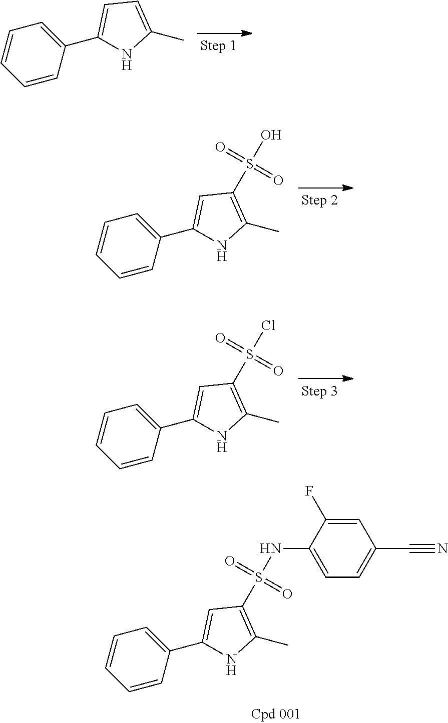 [SMILES: Cc1ccc(-c2ccccc2)[nH]1 ; Cc1[nH]c(-c2ccccc2)cc1S(=O)(=O)O ; Cc1[nH]c(-c2ccccc2)cc1S(=O)(=O)Cl ; Cc1[nH]c(-c2ccccc2)cc1S(=O)(=O)Nc1ccc(C#N)cc1F]

Step 1: A solution of 2-methyl-5-phenyl-1H-pyrrole (800 mg, 5.1 mmol) and Py·SO₃ (1.62 g, 10.2 mmol) in MeCN (20 mL) was stirred 3 h at 120° C. under N₂. The RM was concentrated under reduced pressure. The residue was dissolved in water (100 mL). The aqueous layer was washed with CHCl₃ (3×100 mL). The aqueous layer was concentrated under reduced pressure to afford 1.3 g of 2-methyl-5-phenyl-1H-pyrrole-3-sulfonic acid, which was used without further purification. LCMS (ES−, m/z): [M−H]⁻=236.2.

Step 2: To a stirred solution of 2-methyl-5-phenyl-1H-pyrrole-3-sulfonic acid (1.30 g, 5 mmol) in MeCN (25 mL) was added POCl₃ (4.65 g, 30.5 mmol) dropwise at 0° C. The RM was stirred overnight at 80° C. under N₂ atmosphere. Water was added to the RM at 0° C. The volatiles were removed under reduced pressure. The aqueous layer was extracted with EtOAc (200 mL). The organic layer was concentrated under reduced pressure to afford 1.5 g of 2-methyl-5-phenyl-1H-pyrrole-3-sulfonyl chloride, which was used without further purification.

Step 3: A solution of 2-methyl-5-phenyl-1H-pyrrole-3-sulfonyl chloride (1.40 g, 5 mmol) and 4-amino-3-fluorobenzonitrile (0.78 g, 5.75 mmol) in pyridine (10 mL) was stirred for 2 h at 80° C. under N₂ atmosphere. The RM was concentrated under reduced pressure. The residue was purified by preparative HPLC on a Gemini-NX C18 AXAI Packed Column (21.2×150 mm, 5 μm); Mobile Phase A: Water (0.1% FA), Mobile Phase B: MeCN; Flow rate: 25 mL/min; Gradient: 45% to 55% of B in 10 min. The purification afforded 93 mg (5%) of N-(4-cyano-2-fluorophenyl)-2-methyl-5-phenyl-1H-pyrrole-3-sulfonamide (Cpd 001).

The following compounds were prepared in a similar manner (use of appropriate reagents and purification methods (including chiral HPLC or chiral SFC) known to the person skilled in the art) as described for Cpd 001: Cpd 013; Cpd 019 (from I-007) and Cpd 033.

### Synthesis of N-[2,5-difluoro-4-(trifluoromethyl) phenyl]-5-(2-oxopyrrolidin-1-yl)-1H-pyrrole-3-sulfonamidee (Cpd 245)

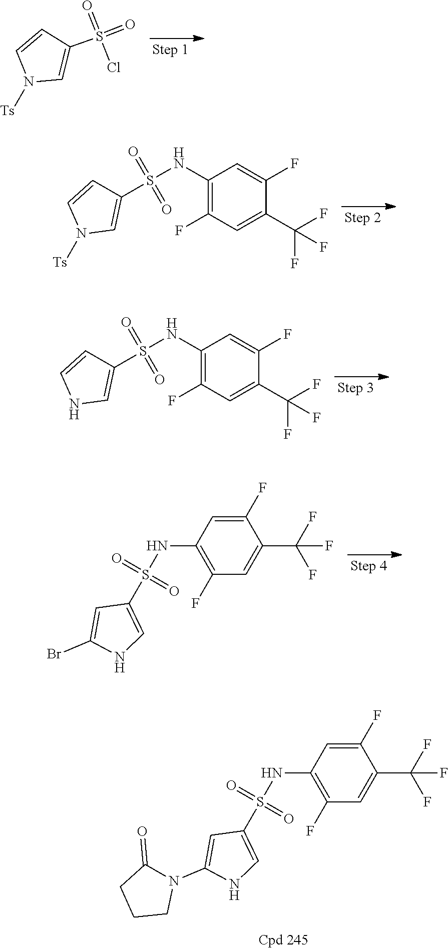 [SMILES: Cc1ccc(S(=O)(=O)n2ccc(S(=O)(=O)Cl)c2)cc1 ; Cc1ccc(S(=O)(=O)n2ccc(S(=O)(=O)Nc3cc(F)c(C(F)(F)F)cc3F)c2)cc1 ; O=S(=O)(Nc1cc(F)c(C(F)(F)F)cc1F)c1cc[nH]c1 ; O=S(=O)(Nc1cc(F)c(C(F)(F)F)cc1F)c1c[nH]c(Br)c1 ; O=C1CCCN1c1cc(S(=O)(=O)Nc2cc(F)c(C(F)(F)F)cc2F)c[nH]1]

Step 1: A solution of 1-(4-methylbenzenesulfonyl) pyrrole-3-sulfonyl chloride (1.0 g, 3.1 mmol) and 2,5-difluoro-4-(trifluoromethyl) aniline (925 mg, 4.7 mmol) in pyridine (15 mL) was stirred for 12 h at 80° C. under N₂ atmosphere. The RM was allowed to cool down to RT. The RM was concentrated under reduced pressure. The residue was purified by FCC over silica gel using as eluent EtOAc/PE (1/3) to afford 1.2 g (80%) of N-[2,5-difluoro-4-(trifluoromethyl)phenyl]-1-(4-methylbenzenesulfonyl)pyrrole-3-sulfonamide. ¹H NMR (400 MHz, CDCl₃) δ 7.78-7.71 (m, 3H), 7.42 (dd, 1H), 7.36-7.31 (m, 2H), 7.25 (dd, 1H), 7.15 (dd, 1H), 6.48 (dd, 1H), 2.43 (s, 3H).

Step 2: A solution of N-[2,5-difluoro-4-(trifluoromethyl) phenyl]-1-(4-m ethylbenzenesulfonyl) pyrrole-3-sulfonamide (1 g, 2.1 mmol) and LiOH (249 mg, 10.4 mmol) in MeOH (20 mL) was stirred for 1 h at RT under N₂ atmosphere. The RM was concentrated under reduced pressure. The residue was purified by FCC over silica gel using as eluent EtOAc/PE (2/5) to afford 620 mg (91%) of N-[2,5-difluoro-4-(trifluoromethyl)phenyl]-1H-pyrrole-3-sulfonamide. ¹H NMR (400 MHz, DMSO-d6) δ 11.70 (s, 1H), 10.65 (s, 1H), 7.71 (m, 1H), 7.52 (m, 1H), 7.45 (dd, 1H), 6.90 (m, 1H), 6.39 (m, 1H).

Step 3: To a solution of N-[2,5-difluoro-4-(trifluoromethyl) phenyl]-1H-pyrrole-3-sulfonamide (500 mg, 1.53 mmol) in DMF (20 mL) was added NBS (272.8 mg, 1.53 mmol) dropwise at −50° C. The RM was stirred for 16 h at −50° C. under argon atmosphere. The RM was concentrated under reduced pressure. The residue was purified by FCC over silica gel using as eluent EtOAc/PE (1/4) to afford 480 mg (77%) of 5-bromo-N-[2,5-difluoro-4-(trifluoromethyl)phenyl]-1H-pyrrole-3-sulfonamide. ¹H NMR (300 MHz, DMSO-d6) δ 12.51 (s, 1H), 10.75 (s, 1H), 7.74 (dd, 1H), 7.61 (dd, 1H), 7.45 (dd, 1H), 6.45 (dd, 1H).

Step 4: To a mixture of 5-bromo-N-[2,5-difluoro-4-(trifluoromethyl) phenyl]-1H-pyrrole-3-sulfonamide (300 mg, 0.740 mmol) and pyrrolidone (63.1 mg, 0.740 mmol) in 1,4-dioxane (4 mL) were added methyl[2-(methylamino) ethyl]amine (13.1 mg, 0.148 mmol) and K₂CO₃ (512 mg, 3.70 mmol) and CuI (28.3 mg, 0.148 mmol) at RT. The RM was stirred for 16 h at 100° C. under N₂ atmosphere. The mixture was allowed to cool down to RT. The RM was filtered, the filter cake was washed with EtOAc (3×20 mL). The filtrate was concentrated under reduced pressure. The residue was purified by preparative HPLC on a Gemini-NX C18 AXAI Packed Column (21.2×150 mm, 5 μm); Mobile Phase A: Water (0.05% NH₃H₂O), Mobile Phase B: MeCN; Flow rate: 25 mL/min; Gradient: 5% of B during 2 min, 5% to 16% of B in 2.5 min. and 16% to 30% of B in 10 min. The purification afforded 120 mg (39%) of N-[2,5-difluoro-4-(trifluoromethyl)phenyl]-5-(2-oxopyrrolidin-1-yl)-1H-pyrrole-3-sulfonamide (Cpd 245).

### Synthesis of N-(4-cyano-2-fluorophenyl)-5-(pyridin-2-ylmethyl)-1H-pyrrole-3-sulfonamide (Cpd 491)

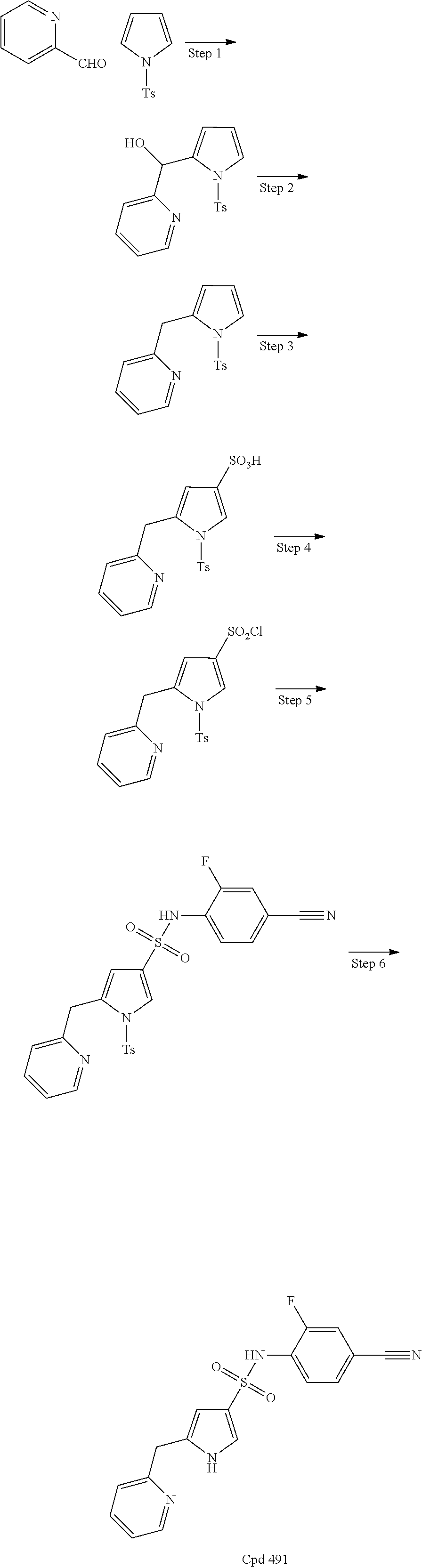

Step 1: To a stirred solution of 1-tosyl-1H-pyrrole (4.0 g, 18.1 mmol) in dry THF (30.0 mL), t-BuLi (1.7 M in pentane) (11.7 mL, 19.9 mmol) was added drop wise at −78° C. and stirred for 20 min at same temperature. After formation of desbromo as evidenced from TLC, solution of picolinaldehyde (1.94 g, 18.1 mmol) in THF (5.0 mL) was added drop wise at −78° C. under inert atmosphere and the reaction mixture was stirred at −78° C. for 4 h. The RM was quenched with saturated aq. NH₄Cl solution and the aqueous phase was extracted with EtOAc. Organic phase was dried over anhydrous Na₂SO₄, filtered and evaporated under reduced pressure. The residue was purified by FCC on silica gel using a gradient of EtOAc (0-6%) in hexane to afford 3.0 g (51%) of pyridin-2-yl(1-tosyl-1H-pyrrol-2-yl)methanol. LCMS (ES+, m/z) [M+H]⁺=329.2. ¹H NMR (400 MHz, DMSO-d6): δ ppm 8.42 (s, 1H), 7.83-7.74 (m, 3H), 7.44-7.31 (m, 4H), 7.27-7.25 (m, 1H), 6.21-6.17 (m, 2H), 5.97-5.95 (m, 1H), 5.75 (s, 1H), 2.37 (s, 3H).

Step 2: In a sealed tube pyridin-2-yl(1-tosyl-1H-pyrrol-2-yl)methanol (1.25 g, 3.8 mmol) was taken in DCE (3.0 mL) and it was degassed with argon for 5 mins. Then triethylsilane (2.43 mL, 15.2 mmol) was added, followed by the addition of TFA (2.33 mL, 30.4 mmol) and the RM was heated at 70° C. After 3 h, the RM was quenched with saturated aq. NaHCO₃solution to adjust the pH to 7 and extracted with DCM. The organic phase was dried over Na₂SO₄, filtered and evaporated under reduced pressure The residue was purified by FCC on silica gel using a gradient of EtOAc (5-10%) in DCM to afford 950 mg (80%) of 2-((1-tosyl-1H-pyrrol-2-yl)methyl)pyridine. LCMS (ES+, m/z) [M+H]⁺=313.84.

Step 3: A stirred solution of 2-((1-tosyl-1H-pyrrol-2-yl)methyl)pyridine (1.1 g, 3.5 mmol) in MeCN (10.0 mL) was cooled to 0° C. Chlorosulfonic acid (1.2 mL, 17.6 mmol) was added drop wise to the RM and the resulting mixture was stirred at 0° C. for 1 h. The solvent was evaporated under reduced pressure. Resulting crude was diluted with 10% MeOH/DCM and neutralized with 10% aq. K₂CO₃ solution. Organic phase was separated, dried over Na₂SO₄ and evaporated under reduced pressure to afford 1.38 g of crude 5-(pyridin-2-ylmethyl)-1-tosyl-1H-pyrrole-3-sulfonic acid that used for the next step without further purification. LC-MS (ES+, m/z) [M+H]⁺=393.2.

Step 4: A stirred solution of 5-(pyridin-2-ylmethyl)-1-tosyl-1H-pyrrole-3-sulfonic acid (1.38 g, 3.5 mmol) in MeCN (10.0 mL) was cooled to 0° C. POCl₃ (1.6 mL, 17.6 mmol) was then added drop wise and the RM was stirred at 80° C. After 3 h, the solvent was evaporated under reduced pressure. Then it was quenched with ice and extracted with 10% MeOH/DCM solution. The organic layer was dried over Na₂SO₄, filtered and evaporated under reduced pressure. The residue was purified by FCC on silica gel using a gradient of MeOH (0-5%) in DCM to afford 800 mg (55%) 5-(pyridin-2-ylmethyl)-1-tosyl-1H-pyrrole-3-sulfonyl chloride that was used in the next step without further purification.

Step 5: In a 10 mL screwed cap vial 5-(pyridin-2-ylmethyl)-1-tosyl-1H-pyrrole-3-sulfonyl chloride (550 mg, 1.3 mmol) and 4-amino-3-fluorobenzonitrile (217.45 mg, 1.6 mmol) was dissolved in MeCN (4.0 mL). Pyridine (0.54 mL, 6.7 mmol) was then added and the reaction mixture was heated at 80° C. After 16 hours, the solvent was evaporated under reduced pressure. The residue was purified by FCC on silica gel using a gradient of DCM (0-70%) in Hexane to afford 270 mg (40%) of N-(4-cyano-2-fluorophenyl)-5-(pyridin-2-ylmethyl)-1-tosyl-1H-pyrrole-3-sulfonamide.

Step 6: To the stirred solution of N-(4-cyano-2-fluorophenyl)-5-(pyridin-2-yl methyl)-1-tosyl-1H-pyrrole-3-sulfonamide (270 mg, 0.5 mmol) in MeOH-Water (1:1, 8.0 mL), 5 M aqueous KOH solution (1.0 mL) was added and the reaction mixture was heated to reflux for 30 minutes. The solvents were removed under reduced pressure. and the crude thus obtained was purified by R^{p} preparative HPLC on a YMC-Actus Triart C18 column (20×250 mm, 5 μm) operating at ambient temperature and flow rate of 16 mL/min; Mobile phase A: 20 mM NH₄HCO₃ in water; Mobile phase B: MeCN; Gradient profile: mobile phase initial composition of 80% A and 20% B for 5 min, then to 40% A and 60% B in 30 min., then to 5% A and 95% B in 31 min., held this composition up to 33 min. for column washing, then returned to initial composition in 34 min. and held till 36 mins. The purification afforded 45 mg (24%) of N-(4-cyano-2-fluorophenyl)-5-(pyridin-2-ylmethyl)-1H-pyrrole-3-sulfonamide (Cpd 491).

### Synthesis of N-(4-cyano-2-fluorophenyl)-5-(pyridin-2-ylmethyl)-1H-pyrrole-3-sulfonamide (Cpd 493)

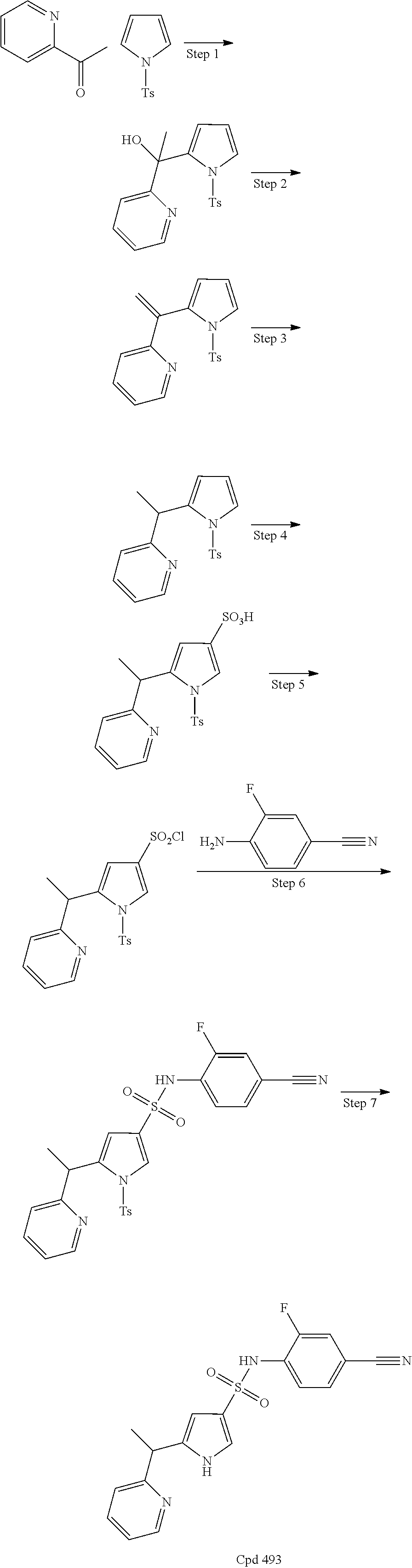

Step 1: A stirred solution of 1-tosyl-1H-pyrrole (4.0 g, 18.1 mmol) in dry THF (40.0 mL) was cooled to −78° C. and treated with t-BuLi (8.5 mL, 19.9 mmol) dropwise. RM was stirred at −78° C. for 2 hours. Then 1-(pyridin-2-yl)ethan-1-one (2.19 g, 18.1 mmol) was dissolved in THF (5.0 mL) and added drop wise to the RM. The resulting mixture was allowed to warm up and stirred at RT. After 16 h, the RM was quenched with saturated aq. NH₄Cl solution and extracted with EtOAc. Organic layer was washed with brine solution, dried over Na₂SO₄, filtered and evaporated under reduced pressure. The residue was purified by FCC on silica gel using a gradient of EtOAc (10-50%) in hexane to afford 1.92 g (31%) of 1-(pyridin-2-yl)-1-(1-tosyl-1H-pyrrol-2-yl)ethan-1-ol. LCMS (ES+, m/z) [M+H]⁺=343.37. ¹H NMR (400 MHz, DMSO-d6): δ ppm 8.37-8.36 (m, 1H), 7.73 (t, 1H), 7.57 (d, 2H), 7.42-7.41 (m, 1H), 7.36-7.30 (m, 3H), 7.25-7.20 (m, 1H), 6.45-6.44 (m, 1H), 6.29 (t, 1H), 5.64 (s, 1H), 2.35 (s, 3H), 1.67 (s, 3H).

Step 2: In a sealed tube 1-(pyridin-2-yl)-1-(1-tosyl-1H-pyrrol-2-yl)ethan-1-ol (2.56 g, 7.5 mmol) was taken in DCE (10.0 mL) and degassed with argon for 5 mins. Then triethylsilane (4.8 mL, 29.9 mmol) was added, followed by the addition of TEA (4.58 mL, 59.8 mmol). The resulting reaction mixture was stirred at 70° C. After 3 h, the RM was quenched with saturated aq. NaHCO₃solution to adjust the pH-7 and extracted with DCM. The organic phase was dried over Na₂SO₄, filtered and evaporated under reduced pressure. The residue was purified by FCC on silica gel using a gradient of EtOAc (5-10%) in DCM to afford 1.49 g (61%) of 2-(1-(1-tosyl-1H-pyrrol-2-yl)vinyl)pyridine. LCMS (ES+, m/z) [M+H]⁺=325.24.

Step 3: A stirred solution of 2-(1-(1-tosyl-1H-pyrrol-2-yl)vinyl)pyridine (1.0 g, 3.1 mmol) in EtOH/EtOAc (1:1, 20 mL) was degassed with argon for 5 mins. Then 10% Pd/C (1 g) was added and the reaction mixture was subjected to hydrogenation at RT for 1 hour. The reaction mixture was filtered through celite bed and the solids were washed with 10% MeOH/DCM. The filtrate was evaporated under reduced pressure. The residue was purified by FCC on silica gel using a gradient of EtOAc (0-5% EtOAc) in DCM to afford 617 mg (61%) of 2-(1-(1-tosyl-1H-pyrrol-2-yl)ethyl)pyridine. LCMS (ES+, m/z) [M+H]⁺=327.12. ¹H NMR (400 MHz, DMSO-d6): δ ppm 8.40 (d, 1H), 7.56-7.49 (m, 3H), 7.38-7.37 (m, 1H), 7.29 (d, 2H), 7.15-7.11 (m, 1H), 6.77 (d, 1H), 6.33 (t, 1H), 6.28-6.27 (m, 1H), 4.68-4.63 (m, 1H), 2.33 (s, 3H), 1.42 (d, 3H).

Step 4: A stirred solution of 2-(1-(1-tosyl-1H-pyrrol-2-yl)ethyl)pyridine (617 mg, 1.9 mmol) in MeCN (10.0 mL) was cooled to 0° C. and Chlorosulfonic acid (0.6 mL, 9.4 mmol) was then added drop wise. Reaction mixture was stirred at 0° C. for 1 hour. The solvent was evaporated under reduced pressure and crude thus obtained was diluted with 10% MeOH/DCM and neutralized with 10% aqueous K₂CO₃ solution. The organic phase was dried over Na₂SO₄, filtered and evaporated under reduced pressure to afford 700 mg of crude 5-(1-(pyridin-2-yl)ethyl)-1-tosyl-1H-pyrrole-3-sulfonic acid that was directly used for the next step without further purification. LCMS (ES+, m/z) [M+H]⁺=407.31.

Step 5: To a stirred solution of 5-(1-(pyridin-2-yl)ethyl)-1-tosyl-1H-pyrrole-3-sulfonic acid (760 mg, 1.9 mmol) in MeCN (10 mL) was added POCl₃ (0.88 mL, 9.4 mmol) dropwise at 0° C. After complete addition, RM was stirred at 80° C. After 5 h, solvent was removed and ice cooled water was added to it. The aqueous phase was extracted with EtOAc. Organic phase was separated, dried over Na₂SO₄, filtered and concentrated under reduced pressure to afford 750 mg of crude 5-(1-(pyridin-2-yl)ethyl)-1-tosyl-1H-pyrrole-3-sulfonyl chloride that was directly used for the next step without further purification. LCMS (ES+, m/z) [M+H]⁺=489.42 (quenched with N-Methyl piperazine).

Step 6: In a 10 mL screwed cap vial 5-(1-(pyridin-2-yl)ethyl)-1-tosyl-1H-pyrrole-3-sulfonyl chloride (790 mg, 1.9 mmol) and 4-amino-3-fluorobenzonitrile (379.64 mg, 2.8 mmol) was dissolved in MeCN (5.0 mL). Then, pyridine (0.75 mL, 9.3 mmol) was added and the RM was stirred at 80° C. After 16 h, solvent was evaporated under reduced pressure. The residue was purified by FCC on silica gel using a gradient of DCM (0-70%) in hexane to afford 200 mg (20%) of N-(4-cyano-2-fluorophenyl)-5-(1-(pyridin-2-yl)ethyl)-1-tosyl-1H-pyrrole-3-sulfonamide. LCMS (ES+, m/z) [M+H]⁺=523.3.

Step 7: To the stirred solution of N-(4-cyano-2-fluorophenyl)-5-(1-(pyridin-2-yl)ethyl)-1-tosyl-1H-pyrrole-3-sulfonamide (270 mg, 0.5 mmol) in MeOH/Water (1:1, 8.0 mL), aq. 5 M KOH solution (1.0 mL) was added and the reaction mixture was heated to reflux for 30 minutes. The volatiles were removed under reduced pressure. The crude thus obtained was purified by R^{p} preparative HPLC on a YMC-Actus Triart C18 column (20×250 mm, 5 μm) operating at ambient temperature and flow rate of 16 mL/min; Mobile phase A: 20 mM NH₄HCO₃ in water; Mobile phase B: MeCN; Gradient profile: mobile phase initial composition of 80% A and 20% B for 5 min, then to 40% A and 60% B in 30 min., then to 5% A and 95% B in 31 min., held this composition up to 33 min. for column washing, then returned to initial composition in 34 min. and held till 36 mins. The purification afforded 22 mg (12%) of N-(4-cyano-2-fluorophenyl)-5-(pyridin-2-ylmethyl)-1H-pyrrole-3-sulfonamide (Cpd 493).

### Synthesis of 4-benzyl-N-(4-cyano-2-fluorophenyl)-1H-pyrrole-3-sulfonamide (Cpd 034)

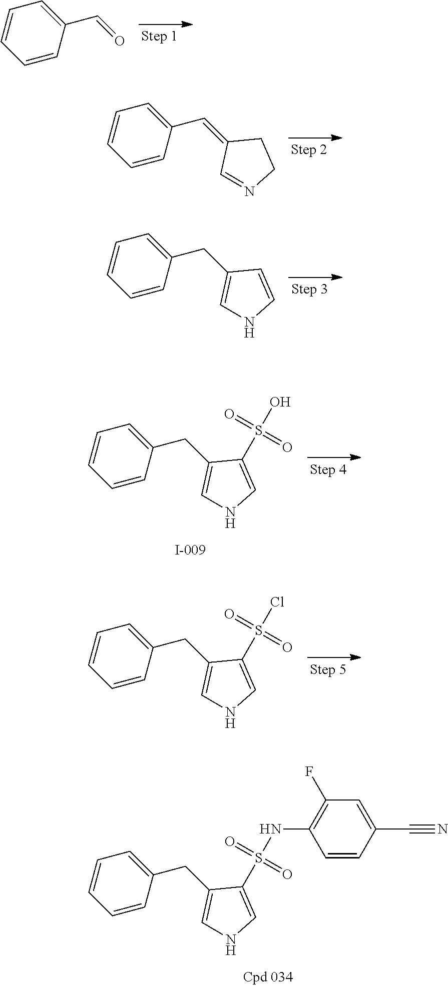 [SMILES: O=Cc1ccccc1 ; C1=NCC/C1=C/c1ccccc1 ; c1ccc(Cc2cc[nH]c2)cc1 ; O=S(=O)(O)c1c[nH]cc1Cc1ccccc1 ; O=S(=O)(Cl)c1c[nH]cc1Cc1ccccc1 ; N#Cc1ccc(NS(=O)(=O)c2c[nH]cc2Cc2ccccc2)c(F)c1]

Step 1: To a solution of benzaldehyde (10 g, 94 mmol) and pyrrolidine (26.8 g, 377 mmol) in m-Xylene (200 mL) was added 3,5-dinitrobenzoic acid (12 g, 56.5 mmol) in portions at RT. The RM was stirred for 20 h at 140° C. under N₂ atmosphere. The RM was concentrated under reduced pressure. The residue was purified by FCC over silica gel using a gradient of EtOAc (30 to 50%) in PE to afford 4 g (27%) of 3-(phenylmethylidene)-4,5-dihydropyrrole. ¹H NMR (300 MHz, CDCl₃) δ 7.90 (q, 1H), 7.52-7.46 (m, 2H), 7.42 (ddd, 2H), 7.34-7.28 (m, 1H), 6.84 (t, 1H), 4.23 (tt, 2H), 2.92-2.81 (m, 2H).

Step 2: To a stirred solution of 3-(phenylmethylidene)-1,2-dihydropyrrole (4 g, 25 mmol) in DMSO (20 mL) was added t-BuOK (3.0 g, 26.7 mmol). The RM was stirred for 2 h at RT under N₂ atmosphere. The RM was concentrated under reduced pressure. The residue was purified by R^{p} FCC on C18 gel using a gradient of MeCN (10 to 70%) in water with 0.1% FA to afford 2 g (50%) of 3-benzyl-1H-pyrrole. ¹H NMR (300 MHz, CDCl₃) δ 8.03 (brs, 1H), 7.34-7.22 (m, 5H), 6.76 (q, 1H), 6.57 (qd, 1H), 6.11 (q, 1H), 3.88 (s, 2H).

Step 3: To a solution of 3-benzyl-1H-pyrrole (500 mg, 3.18 mmol) and Py·SO₃ (556 mg, 3.50 mmol) in MeCN (10 mL) was stirred for 8 h at 120° C. under nitrogen atmosphere. After cooling down to RT, the resulting mixture was used directly in next step without further purification.

Step 4: To a solution of 4-benzyl-1H-pyrrole-3-sulfonic acid (I-009) (3.18 mmol) in MeCN (10 mL) was added POCl₃ (1.62 g, 10.5 mmol) dropwise at RT. After completion, the RM was concentrated under reduced pressure to afford 900 mg of 4-benzyl-1H-pyrrole-3-sulfonyl chloride, which was used without further purification.

Step 5: To a solution of 4-benzyl-1H-pyrrole-3-sulfonyl chloride (900 mg, 2.3 mmol), 4-amino-3-fluorobenzonitrile (479 mg, 3.52 mmol) and pyridine (1.86 g, 23.46 mmol) in MeCN (20 mL) was stirred overnight at RT. The RM was concentrated under reduced pressure. The residue was purified by R^{p} FCC on C18 gel using a gradient of MeCN (50 to 80%) in water with 0.1% FA. The residue was purified by preparative TLC (hexane/EtOAc=3/1) to afford 67 mg (8%) of 4-benzyl-N-(4-cyano-2-fluorophenyl)-1H-pyrrole-3-sulfonamide (Cpd 034).

The following compounds were prepared in a similar manner (use of appropriate reagents and purification methods (including chiral HPLC or chiral SFC) known to the person skilled in the art) as described for Cpd 034: Cpd 151; Cpd 152; Cpd 153; Cpd 158; Cpd 161; Cpd 162; Cpd 163; Cpd 171; Cpd 177; Cpd 182; Cpd 184; Cpd 192; Cpd 193; Cpd 194; Cpd 206; Cpd 208; Cpd 209; Cpd 210; Cpd 211; Cpd 271; Cpd 306; Cpd 307; Cpd 308; Cpd 309; Cpd 340; Cpd 341; Cpd 342; Cpd 343; Cpd 367 and Cpd 368; Cpd 426; Cpd 427; Cpd 431; Cpd 432; Cpd 433; Cpd 439; Cpd 440; Cpd 444; Cpd 468 and Cpd 501.

### Synthesis of Synthesis of 4-benzyl-5-chloro-N-(4-cyano-2-fluorophenyl)-1H-pyrrole-3-sulfonamide (Cpd 159)

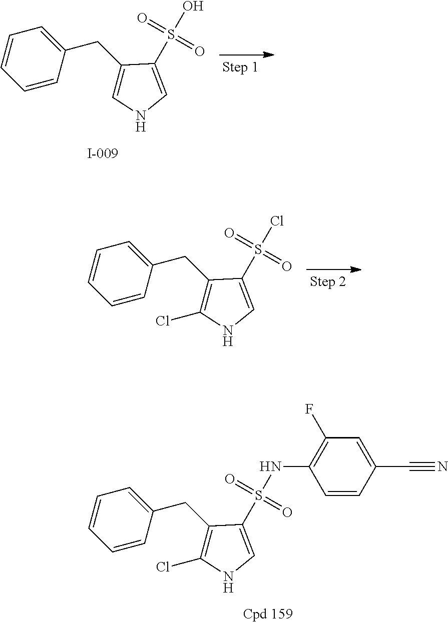 [SMILES: O=S(=O)(O)c1c[nH]cc1Cc1ccccc1 ; O=S(=O)(Cl)c1c[nH]c(Cl)c1Cc1ccccc1 ; N#Cc1ccc(NS(=O)(=O)c2c[nH]c(Cl)c2Cc2ccccc2)c(F)c1]

Step 1: To a mixture of 4-benzyl-1H-pyrrole-3-sulfonic acid (I-009) (1.00 g, 4.20 mmol) in MeCN (15 mL) was added POCl₃ (4.0 mL, 42 mmol) at RT under argon atmosphere. The RM was stirred for 3 h at 70° C. under argon atmosphere. The RM was allowed to cool down to RT. The reaction was quenched with water. The resulting mixture was extracted with DCM (3×100 mL). The combined organic layers were washed with brine (3×50 mL), dried over Na₂SO₄, filtrated, and concentrated under reduced pressure to afford 400 mg (37%) of 4-benzyl-5-chloro-1H-pyrrole-3-sulfonyl chloride.

Step 2: To a solution of 4-amino-3-fluorobenzonitrile (256 mg, 1.76 mmol) in pyridine (8 mL) was added 4-benzyl-5-chloro-1H-pyrrole-3-sulfonyl chloride (300 mg, 1.17 mmol) at RT. The RM was stirred for 12 h at 80° C. under argon atmosphere. The mixture was allowed to cool down to RT and concentrated under reduced pressure. The residue was purified by preparative HPLC on a Gemini-NX C18 AXAI Packed Column (21.2×150 mm, 5 μm); Mobile Phase A: Water (0.1% FA), Mobile Phase B: MeCN; Flow rate: 25 mL/min; Gradient: 10% of B during 10 min., then 10% to 39% of B in 2.5 min and then 39% to 72% of B in 10.5 min. The purification afforded 142 mg (32%) of 4-benzyl-5-chloro-N-(4-cyano-2-fluorophenyl)-1H-pyrrole-3-sulfonamide (Cpd 159).

### Synthesis of 4-benzoyl-N-(4-cyano-2-fluoro-phenyl)-1H-pyrrole-3-sulfonamide (Cpd 195) and N-(4-cyano-2-fluoro-phenyl)-4-[hydroxy(phenyl)methyl]-1H-pyrrole-3-sulfonamide (Cpd 207)

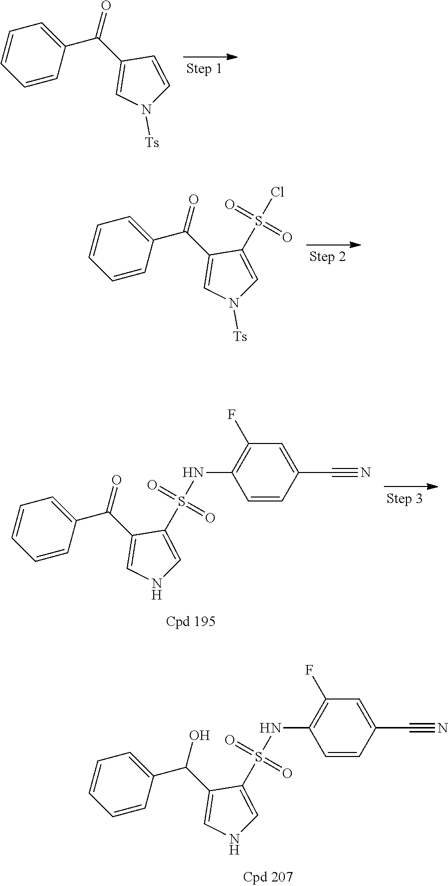 [SMILES: Cc1ccc(S(=O)(=O)n2ccc(C(=O)c3ccccc3)c2)cc1 ; Cc1ccc(S(=O)(=O)n2cc(C(=O)c3ccccc3)c(S(=O)(=O)Cl)c2)cc1 ; N#Cc1ccc(NS(=O)(=O)c2c[nH]cc2C(=O)c2ccccc2)c(F)c1 ; N#Cc1ccc(NS(=O)(=O)c2c[nH]cc2C(O)c2ccccc2)c(F)c1]

Step 1: HSO₃Cl (1.79 g, 15.37 mmol) was added to 3-benzoyl-1-(4-methylbenzenesulfonyl) pyrrole (1.00 g, 3.07 mmol) at 0° C. The RM was stirred for 12 h at 80° C. under argon atmosphere. The reaction was quenched with water at 0° C. The aqueous mixture was extracted with DCM (3×200 mL). The combined organic layers were washed with brine (3×100 mL), dried over Na₂SO₄, filtrated, and concentrated to afford 800 mg (61%) of 4-benzoyl-1-(4-methyl benzenesulfonyl) pyrrole-3-sulfonyl chloride.

Step 2: To a solution of 4-amino-3-fluorobenzonitrile (144.5 mg, 1.06 mmol) in pyridine (8 mL) was added 4-benzoyl-1-(4-methyl benzenesulfonyl) pyrrole-3-sulfonyl chloride (300 mg, 0.708 mmol). The RM was stirred for 12 h at 80° C. under argon atmosphere. The mixture was allowed to cool down to RT and concentrated under reduced pressure. The residue was purified by preparative HPLC on XSelect CSH Prep C18 OBD Column (19×250 mm, 5 μm); Mobile Phase A: Water (0.05% FA), Mobile Phase B: MeCN; Flow rate: 25 mL/min; Gradient: 30% to 59% of B in 10 min. The purification afforded 110 mg (42%) of 4-benzoyl-N-(4-cyano-2-fluorophenyl)-1H-pyrrole-3-sulfonamide (Cpd 195).

Step 3: To a stirred solution of 4-benzoyl-N-(4-cyano-2-fluorophenyl)-1H-pyrrole-3-sulfonamide (Cpd 195) (300 mg, 0.812 mmol) in THF (6 mL) was added NaBH₄ (154 mg, 4.1 mmol). The RM was stirred 3 h at RT under argon atmosphere. The RM was concentrated under reduced pressure. The residue was purified by R^{p} FCC on C18 gel using a gradient of MeCN (10 to 50%) in water to afford 160 mg (53%) of N-(4-cyano-2-fluorophenyl)-4-[hydroxy(phenyl)methyl]-1H-pyrrole-3-sulfonamide (Cpd 207).

### Synthesis of N-(4-cyano-2-fluorophenyl)-4-(1-phenylethyl)-1H-pyrrole-3-sulfonamide (Cpd 225) from Cpd 195

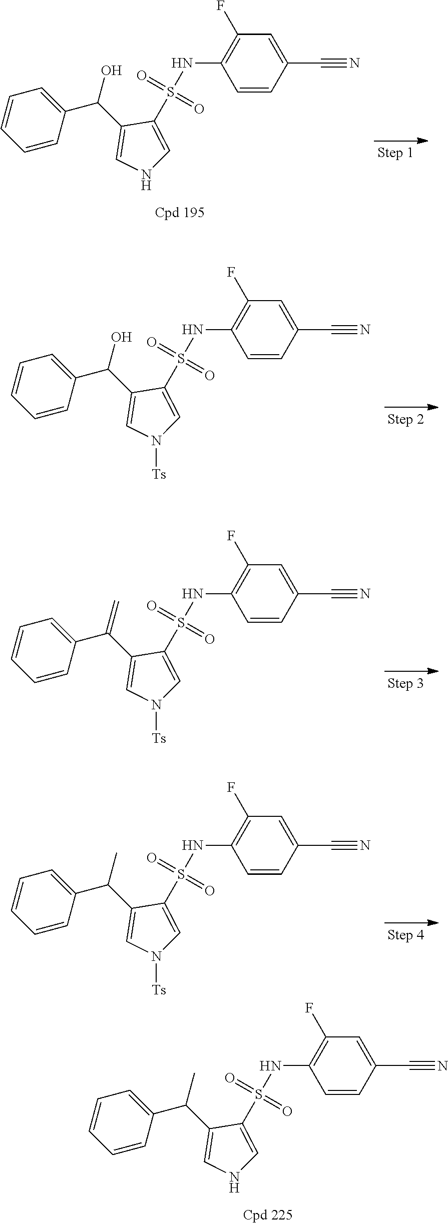 [SMILES: N#Cc1ccc(NS(=O)(=O)c2c[nH]cc2C(O)c2ccccc2)c(F)c1 ; Cc1ccc(S(=O)(=O)n2cc(C(O)c3ccccc3)c(S(=O)(=O)Nc3ccc(C#N)cc3F)c2)cc1 ; C=C(c1ccccc1)c1cn(S(=O)(=O)c2ccc(C)cc2)cc1S(=O)(=O)Nc1ccc(C#N)cc1F ; Cc1ccc(S(=O)(=O)n2cc(C(C)c3ccccc3)c(S(=O)(=O)Nc3ccc(C#N)cc3F)c2)cc1 ; CC(c1ccccc1)c1c[nH]cc1S(=O)(=O)Nc1ccc(C#N)cc1F]

Step 1: To a solution of 4-benzoyl-N-(4-cyano-2-fluorophenyl)-1H-pyrrole-3-sulfonamide (Cpd 195) (2.0 g, 5.41 mmol) in THF (30 mL) was added NaH (60% in mineral oil) (650 mg, 16.2 mmol) at 0° C. The RM was stirred for 1 h at RT under argon atmosphere. TsCl (2.06 g, 10.8 mmol) was added. The RM was stirred for 12 h at RT. The reaction was quenched by water (100 mL) and extracted with EtOAc (3×200 mL). The combined organic layers were washed with brine (3×100 mL), dried over Na₂SO₄, filtrated, concentrated under reduced pressure. The residue was purified by FCC over silica gel using as eluent EtOAc/PE (1/3) to afford 2.4 g (84%) of 4-benzoyl-N-(4-cyano-2-fluorophenyl)-1-tosyl-1H-pyrrole-3-sulfonamide. ¹H NMR (300 MHz, DMSO-d6) δ 10.19 (s, 1H), 8.18 (d, J=2.4 Hz, 1H), 8.07-7.97 (m, 2H), 7.95 (d, J=2.4 Hz, 1H), 7.84-7.66 (m, 4H), 7.62-7.50 (m, 4H), 7.48 (d, J=8.2 Hz, 2H), 2.44 (s, 3H).

Step 2: To a mixture of methyltriphenylphosphaniumbromide (4.8 g, 13.4 mmol) in THF (40 mL) was added Butyl lithium (4.6 mL, 11.6 mmol, 2.5 N) dropwise at −78° C. under argon atmosphere. The RM was stirred for 1 h at −50° C. Then, 4-benzoyl-N-(4-cyano-2-fluorophenyl)-1-(4-methylbenzenesulfonyl) pyrrole-3-sulfonamide (2.0 g, 3.82 mmol) was added. The RM was stirred for 16 h at RT under argon atmosphere. The reaction was quenched by saturated aq. NH₄Cl (10 mL). The RM was concentrated under reduced pressure. The residue was purified by FCC over silica gel using as eluent EtOAc/PE (1/4) to afford 0.5 g (25%) of N-(4-cyano-2-fluorophenyl)-1-(4-methyl benzenesulfonyl)-4-(1-phenylethenyl)pyrrole-3-sulfonamide. ¹H NMR (400 MHz, DMSO-d6) δ 10.76 (s, 1H), 8.05 (d, 1H), 7.99-7.95 (m, 2H), 7.81 (d, 1H), 7.67 (dd, 1H), 7.51-7.45 (m, 3H), 7.38 (d, 1H), 7.34 (d, 1H), 7.21 (dd, 2H), 7.03 (m, 2H), 5.73 (s, 1H), 5.32 (s, 1H), 2.45 (s, 3H).

Step 3: To a stirred solution of N-(4-cyano-2-fluorophenyl)-1-(4-methyl benzenesulfonyl)-4-(1-phenylethenyl) pyrrole-3-sulfonamide (400 mg, 0.77 mmol) in MeOH (10 mL) was added Pd/C (5%, 200 mg) under N₂ atmosphere. The RM was stirred for 2 h at RT under hydrogen atmosphere. The RM was filtered through a Celite pad and concentrated under reduced pressure. The residue was purified by FCC over silica gel using as eluent EtOAc/PE (1/3) to afford 0.3 g (73%) of N-(4-cyano-2-fluorophenyl)-1-(4-methyl benzenesulfonyl)-4-(1-phenylethyl)pyrrole-3-sulfonamide.

Step 4: A mixture of N-(4-cyano-2-fluorophenyl)-1-(4-methyl benzenesulfonyl)-4-(1-phenylethyl)pyrrole-3-sulfonamide (300 mg, 0.57 mmol) and LiOH·H₂O (68.6 mg, 2.86 mmol) in MeOH (2 mL) and H₂O (1 mL) was stirred for 1 h at RT under nitrogen atmosphere. The RM was concentrated under reduced pressure. The residue was purified by R^{p} FCC on C18 gel using a gradient of MeCN (10 to 65%) in water to afford 120 mg (56%) of N-(4-cyano-2-fluorophenyl)-4-(1-phenylethyl)-1H-pyrrole-3-sulfonamide (Cpd 225).

### Synthesis of 4-benzyl-N-(4-cyano-2-fluorophenyl)-5-methyl-1H-pyrrole-3-sulfonamide (Cpd 067) from Cpd 034

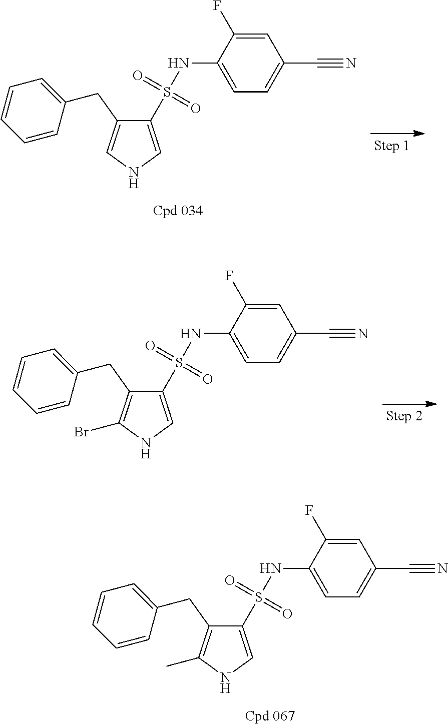 [SMILES: N#Cc1ccc(NS(=O)(=O)c2c[nH]cc2Cc2ccccc2)c(F)c1 ; N#Cc1ccc(NS(=O)(=O)c2c[nH]c(Br)c2Cc2ccccc2)c(F)c1 ; Cc1[nH]cc(S(=O)(=O)Nc2ccc(C#N)cc2F)c1Cc1ccccc1]

Step 1: To a solution of 4-benzyl-N-(4-cyano-2-fluorophenyl)-1H-pyrrole-3-sulfonamide (Cpd 034) (380 mg, 1.07 mmol) in DMF (5 mL) was added NBS (190 mg, 1.07 mmol) at 0° C. The RM was stirred for 1 h at RT under N₂ atmosphere. The RM was concentrated under reduced pressure. The residue was purified by R^{p} FCC on C18 gel using a gradient of MeCN (10 to 50%) in water to afford 250 mg (54%) of 4-benzyl-5-bromo-N-(4-cyano-2-fluorophenyl)-1H-pyrrole-3-sulfonamide (250 mg, 53.8%). ¹H NMR (400 MHz, DMSO-d6) δ 12.55-12.40 (m, 1H), 10.64 (s, 1H), 7.74 (dd, 1H), 7.58 (d, 1H), 7.54 (dd, 1H), 7.47 (t, 1H), 7.19-7.01 (m, 5H), 3.90 (s, 2H).

Step 2: To a mixture of trimethyl-1,3,5,2,4,6-trioxatriborinane (156 mg, 1.24 mmol), 4-benzyl-5-bromo-N-(4-cyano-2-fluorophenyl)-1H-pyrrole-3-sulfonamide (270 mg, 0.62 mmol) and K₂CO₃ (258 mg, 1.86 mmol) in THF (10 mL) and H₂O (2 mL) was added Pd(dppf)Cl₂ (46 mg, 0.06 mmol) at RT. The RM was stirred overnight at 80° C. under N₂ atmosphere. The RM was diluted with H₂O (30 mL), then extracted with EtOAc (3×20 mL). The combined organic layers were washed with brine (3×20 mL), dried over Na₂SO₄, filtrated, and concentrated under reduced pressure. The residue was purified by R^{p} FCC on C18 gel using a gradient of MeCN (30 to 50%) in water to afford 53.7 mg (23%) of 4-benzyl-N-(4-cyano-2-fluorophenyl)-5-methyl-1H-pyrrole-3-sulfonamide (Cpd 067).

### Synthesis of N-(4-cyano-2-fluorophenyl)-4-[(2-methylphenyl) methyl]-1H-pyrrole-3-sulfonamide Cpd 384)

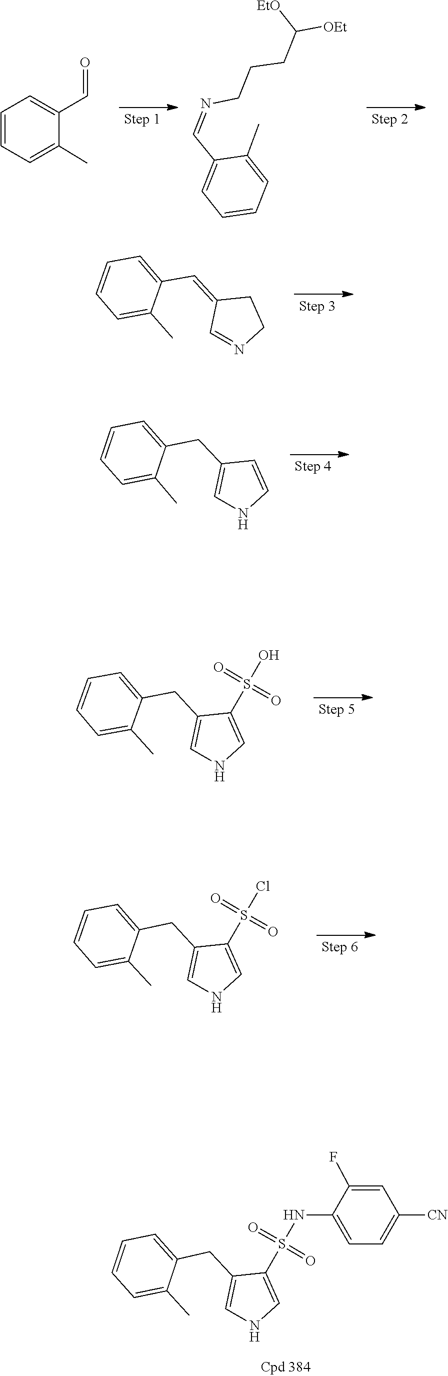

Step 1: A mixture of 2-methylbenzaldehyde (5 g, 41.61 mmol) and 4,4-diethoxy-butylamine (6.71 g, 41.61 mmol) in CHCl₃ (50 mL) was stirred for 6 h at RT under N₂ atmosphere. The resulting mixture was concentrated under vacuum to afford 9.2 g (84%) of crude (Z)-(4,4-diethoxybutyl) [(2-methylphenyl) methylidene]amine that was used in subsequent step without further purification. LCMS (ES+, m/z) [M+H]⁺=264.1. ¹H NMR (300 MHz, CHCl₃) δ 8.60 (d, J=1.4 Hz, 1H), 7.87 (dd, J=7.6, 1.7 Hz, 1H), 7.36-7.18 (m, 2H), 7.23-7.14 (m, 1H), 4.57 (m, 1H), 3.76-3.59 (m, 4H), 3.52 (m, 2H), 2.52 (s, 2H), 1.90-1.62 (m, 4H), 1.23 (m, 6H).

Step 2: A mixture of (Z)-(4,4-diethoxybutyl) [(2-methylphenyl) methylidene]amine (9.2 g, 34.93 mmol) and TsOH (600 mg, 3.49 mmol) in ortho-xylene (90 mL) was stirred for 40 h at 140° C. under N₂ atmosphere. The mixture was allowed to cool down to RT and was concentrated under vacuum. The residue was purified by FCC on silica gel eluting with PE/EtOAc (1:1) to afford 3.2 g (53%) of (3Z)-3-[(2-methylphenyl) methylidene]-4,5-dihydropyrrole. LCMS (ES+, m/z) [M+H]⁺=172.2. ¹H NMR (300 MHz, CHCl₃) δ 7.93 (m, 1H), 7.55-7.45 (m, 1H), 7.24 (m, 3H), 7.01 (m, 1H), 4.23-4.10 (m, 2H), 2.85-2.74 (m, 2H), 2.41 (s, 3H).

Step 3: A mixture of (3Z)-3-[(2-methylphenyl) methylidene]-4,5-dihydropyrrole (3.2 g, 18.68 mmol) and t-BuOK (2.1 g, 18.68 mmol) in DMSO (40 mL) was stirred for 8 h at RT under N₂ atmosphere. The resulting mixture was extracted with CH₂Cl₂ (3×200 mL), dried over anhydrous Na₂SO₄, filtered and concentrated under reduced pressure. The residue was purified by FCC on silica gel eluting with PE/EtOAc (10:1) to afford 3 g (94%) of 3-[(2-methylphenyl) methyl]-1H-pyrrole. LCMS (ES+, m/z) [M+H]⁺=172.2. ¹H NMR (400 MHz, CHCl₃) δ 7.99 (s, 1H), 7.21-7.14 (m, 1H), 7.17-7.06 (m, 3H), 6.72 (m, 1H), 6.47-6.41 (m, 1H), 6.06 (m, 1H), 3.83 (s, 2H), 2.31 (s, 3H).

Step 4: To a stirred solution of 3-[(2-methylphenyl) methyl]-1H-pyrrole (3 g, 17.52 mmol) in pyridine (180 mL) was added Py·SO₃ (2.79 g, 17.52 mmol) at RT under argon atmosphere. The resulting mixture was stirred for 3 h at 100° C. The mixture was allowed to cool down to RT and was concentrated under reduced pressure. Resulting crude was suspended in water and extracted with CHCl₃ (3×200 mL). The aqueous phase was concentrated under reduced pressure to afford 3.6 g (82%) of 4-[(2-methylphenyl) methyl]-1H-pyrrole-3-sulfonic acid. LCMS (ES−, m/z) [M−H]⁻=250.0.

Step 5: To a stirred solution of 4-[(2-methylphenyl) methyl]-1H-pyrrole-3-sulfonic acid (3.6 g, 14.32 mmol) in MeCN (30 mL) was added POCl₃ (2.64 g, 17.19 mmol) dropwise at RT under argon atmosphere. The resulting mixture was stirred for 3 h at 70° C. The mixture was allowed to cool down to RT and was quenched with water. Aqueous mixture was extracted with CH₂Cl₂ (3×300 mL). The combined organic layers were washed with brine (3×100 mL), dried over anhydrous Na₂SO₄, filtered and concentrated under reduced pressure to afford 1.1 g (28%) of 4-[(2-methylphenyl) methyl]-1H-pyrrole-3-sulfonyl chloride. LCMS (ES−, m/z) [M−H]−=267.9.

Step 6: A mixture of 4-[(2-methylphenyl) methyl]-1H-pyrrole-3-sulfonyl chloride (600 mg, 2.22 mmol) and 4-amino-3-fluorobenzonitrile (454 mg, 3.33 mmol) in pyridine (10 mL) was stirred for 8 h at 80° C. under argon atmosphere. The mixture was allowed to cool down to RT and concentrated under vacuum. The residue was purified by R^{p} FCC on C18 silica gel using a gradient of MeCN (20-50%) in water to afford 100 mg (12%) of N-(4-cyano-2-fluorophenyl)-4-[(2-methylphenyl) methyl]-1H-pyrrole-3-sulfonamide (Cpd 384).

The following compounds were prepared in a similar manner (use of appropriate reagents and purification methods (including chiral HPLC or chiral SFC) known to the person skilled in the art) as described for Cpd 384: Cpd 385; Cpd 386; Cpd 387; Cpd 388; Cpd 389; Cpd 391; Cpd 392; Cpd 407; Cpd 415 (from I-017); Cpd 416; Cpd 417; Cpd 418; Cpd 452; Cpd 453; Cpd 454; Cpd 480; Cpd 481; Cpd 482; Cpd 483; Cpd 484; Cpd 485; Cpd 486; Cpd 535; Cpd 536; Cpd 545; Cpd 546; Cpd 547; Cpd 548; Cpd 558; Cpd 568; Cpd 569; Cpd 574; Cpd 584; Cpd 585; Cpd 605; Cpd 608; 1-036.

### Synthesis of N-(4-cyano-2-fluorophenyl)-4-(3-(dimethylamino) benzyl)-1H-pyrrole-3-sulfonamide (Cpd 390)

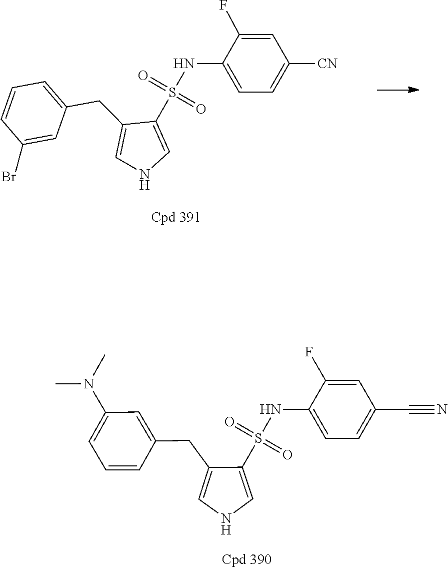 [SMILES: N#Cc1ccc(NS(=O)(=O)c2c[nH]cc2Cc2cccc(Br)c2)c(F)c1 ; CN(C)c1cccc(Cc2c[nH]cc2S(=O)(=O)Nc2ccc(C#N)cc2F)c1]

To a stirred mixture of 4-[(3-bromophenyl) methyl]-N-(4-cyano-2-fluorophenyl)-1H-pyrrole-3-sulfonamide (Cpd 391) (300 mg, 0.69 mmol) and [(2,6-dimethylphenyl) carbamoyl]formic acid (134 mg, 0.69 mmol) in DMSO (3 mL) were added K₃PO₄ (147 mg, 0.69 mmol), dimethylamine (64 mg, 1.38 mmol) and CuI (132 mg, 0.69 mmol) at RT under argon atmosphere. The resulting mixture was stirred for 48 h at 100° C. The mixture was allowed to cool down to RT. Filtered the reaction mixture and washed the filter cake with MeOH (3×10 mL). The filtrate was concentrated under reduced pressure. The residue was purified by R^{p} FCC on C18 silica gel using a gradient of MeCN (20-50%) in water to afford 72 mg (24%) of N-(4-cyano-2-fluorophenyl)-4-{[3-(dimethylamino) phenyl]methyl}-1H-pyrrole-3-sulfonamide (Cpd 390).

### Synthesis of 4-[(3-acetylphenyl) methyl]-N-(4-cyano-2-fluorophenyl)-1H-pyrrole-3-sulfonamide Cpd 490)

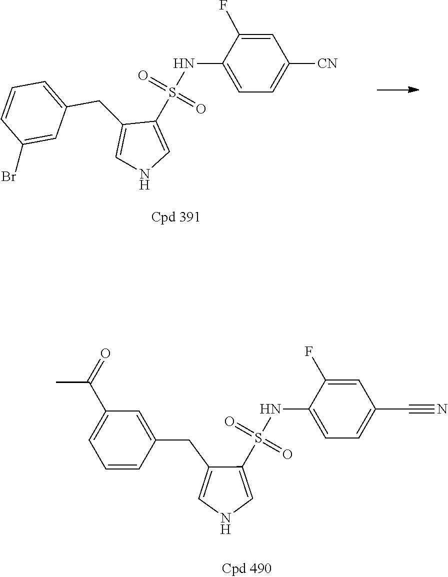 [SMILES: N#Cc1ccc(NS(=O)(=O)c2c[nH]cc2Cc2cccc(Br)c2)c(F)c1 ; CC(=O)c1cccc(Cc2c[nH]cc2S(=O)(=O)Nc2ccc(C#N)cc2F)c1]

To a stirred solution of 4-[(3-bromophenyl) methyl]-N-(4-cyano-2-fluorophenyl)-1H-pyrrole-3-sulfonamide (Cpd 391) (200 mg, 0.461 mmol) and butyl vinyl ether (231 mg, 2.31 mmol) and [3-(diphenylphosphanyl) propyl]diphenylphosphane (19 mg, 0.046 mmol) in [bmim][BF₄] (2 mL) were added triethylamine (56 mg, 0.553 mmol) and Pd(OAc)₂ (5 mg, 0.023 mmol) at RT under N₂ atmosphere. The resulting mixture was stirred for 36 h at 115° C. The mixture was allowed to cool down to RT. To the above mixture was added aq. HCl (10 mL) and H₂O (10 mL). The resulting mixture was stirred for additional 30 min at RT. The mixture was extracted with EtOAc (3×100 mL). The combined organic layers were washed with brine (3×50 mL), dried over anhydrous Na₂SO₄, filtered and concentrated under reduced pressure. The residue was purified by preparative HPLC on a Sunfire Prep C18 Column (30×150 mm, 5 μm); Mobile Phase A: water (0.1% FA), Mobile Phase B: MeCN; Flow rate: 60 mL/min; Gradient: 35% B to 50% B in 7 min, 50% B. The purification afforded 70 mg (38%) of 4-[(3-acetylphenyl) methyl]-N-(4-cyano-2-fluorophenyl)-1H-pyrrole-3-sulfonamide (Cpd 490).

### Synthesis of N-(4-cyano-2,5-difluorophenyl)-4-[(3-cyclopropylphenyl)methyl]-1H-pyrrole-3-sulfonamide (Cpd 586)

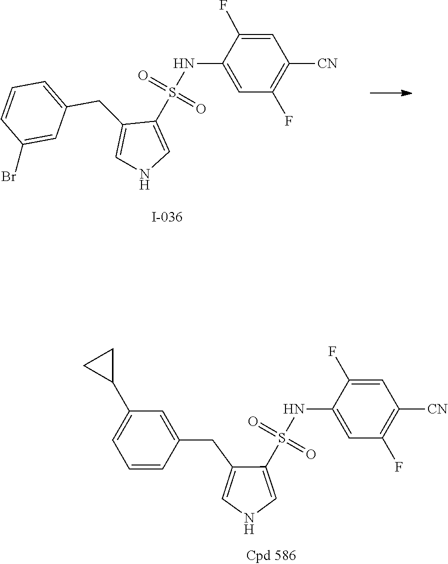 [SMILES: N#Cc1cc(F)c(NS(=O)(=O)c2c[nH]cc2Cc2cccc(Br)c2)cc1F ; N#Cc1cc(F)c(NS(=O)(=O)c2c[nH]cc2Cc2cccc(C3CC3)c2)cc1F]

To a stirred mixture of 4-[(3-bromophenyl) methyl]-N-(4-cyano-2,5-difluorophenyl)-1H-pyrrole-3-sulfonamide (I-036) (500 mg, 1.10 mmol) and cyclopropylboronic acid (124 mg, 1.44 mmol) in dioxane (10 mL) and H₂O (1 mL) were added K₂CO₃ (459 mg, 3.32 mmol) and Pd(dppf)Cl₂ (81 mg, 0.110 mmol) at RT under nitrogen atmosphere. The resulting mixture was stirred for 12 h at 90° C. under N₂ atmosphere. The mixture was allowed to cool down to RT. The resulting mixture was concentrated under vacuum. The crude product was purified by preparative HPLC on a Xselect CSH 018 OBD Column (30×150 mm, 5 μm); Mobile Phase A: Water (0.1% FA), Mobile Phase B: MeCN; Flow rate: 60 mL/min; Gradient: 45% B to 70% B in 7 min, 70% B. The purification afforded 70 mg (15%) of N-(4-cyano-2,5-difluorophenyl)-4-[(3-cyclopropylphenyl) methyl]-1H-pyrrole-3-sulfonamide (Cpd 586).

The following compounds were prepared in a similar manner (use of appropriate reagents and purification methods (including chiral HPLC or chiral SFC) known to the person skilled in the art) as described for Cpd 586: Cpd 590.

### Synthesis of 4-benzyl-N-(4-cyano-2-fluorophenyl)-5-fluoro-1H-pyrrole-3-sulfonamide (Cpd 609)

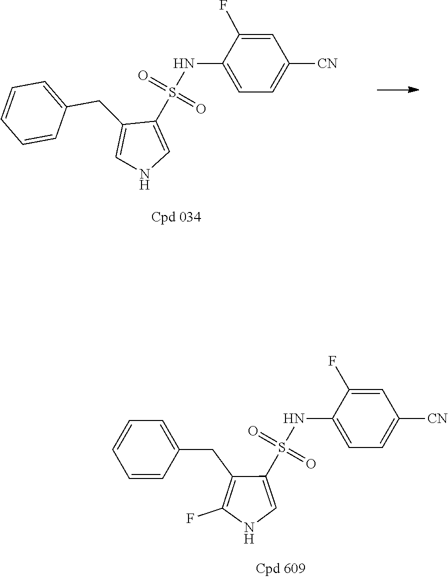 [SMILES: N#Cc1ccc(NS(=O)(=O)c2c[nH]cc2Cc2ccccc2)c(F)c1 ; N#Cc1ccc(NS(=O)(=O)c2c[nH]c(F)c2Cc2ccccc2)c(F)c1]

A mixture of 4-benzyl-N-(4-cyano-2-fluorophenyl)-1H-pyrrole-3-sulfonamide (Cpd 034) (300 mg, 0.844 mmol) and 1-chloromethyl-4-fluoro-1,4-diazoniabicyclo[2.2.2]octane bis(tetrafluoroborate) (299 mg, 0.844 mmol) in EtOAc (5 mL) was stirred for 24 h at 65° C. under N₂ atmosphere. The mixture was allowed to cool down to RT. The resulting mixture was concentrated under vacuum. The residue was purified by R^{p} FCC on C18 silica gel using a gradient of MeCN (10-50%) in water (0.1% FA) to afford 60 mg (18%) of 4-benzyl-N-(4-cyano-2-fluorophenyl)-5-fluoro-1H-pyrrole-3-sulfonamide (Cpd 609).

### Synthesis of N-(4-cyano-2-fluorophenyl)-4-(pyridin-2-ylmethyl)-1H-pyrrole-3-sulfonamide (Cpd 623)

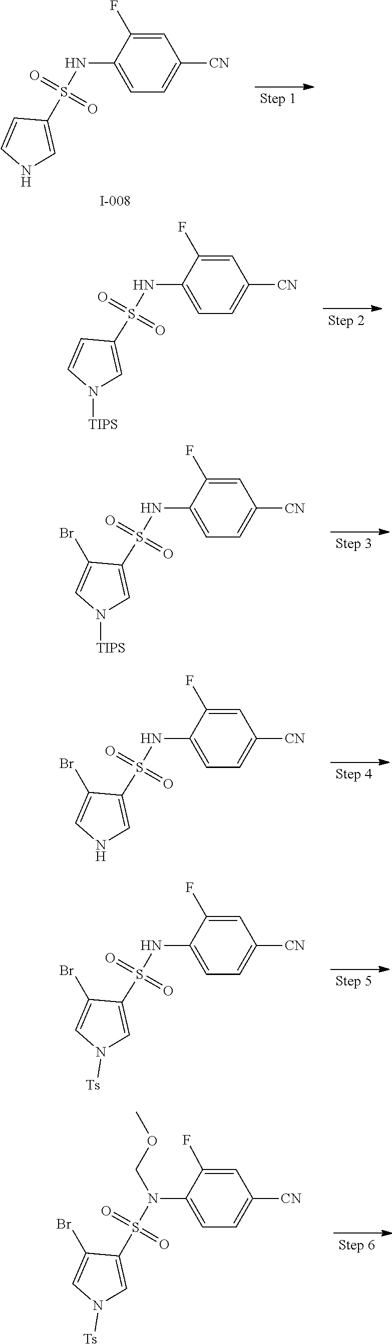

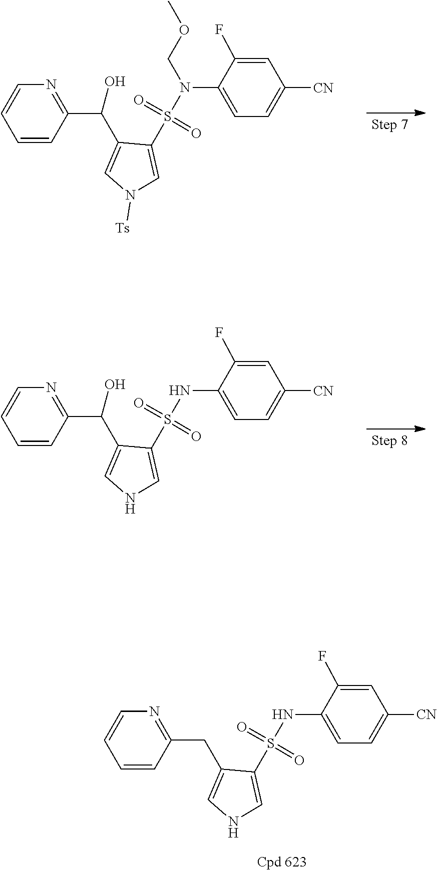 [SMILES: COCN(c1ccc(C#N)cc1F)S(=O)(=O)c1cn(S(=O)(=O)c2ccc(C)cc2)cc1C(O)c1ccccn1 ; N#Cc1ccc(NS(=O)(=O)c2c[nH]cc2C(O)c2ccccn2)c(F)c1 ; N#Cc1ccc(NS(=O)(=O)c2c[nH]cc2Cc2ccccn2)c(F)c1]

Step 1: To a stirred solution of N-(4-cyano-2-fluorophenyl)-1H-pyrrole-3-sulfonamide (I-008) (9.1 g, 34.31 mmol) in THF (150 mL) were added (i-Pr₃)SiCl (9.67 g, 41.17 mmol) and NaH (1.23 g, 51.46 mmol) at 0° C. under N₂ atmosphere. The resulting mixture was stirred for 12 h at RT under N₂ atmosphere. The reaction was quenched with saturated aq. NH₄Cl at RT. The resulting mixture was extracted with EtOAc (3×500 mL). The combined organic layers were washed with brine (3×300 mL) and concentrated under reduced pressure. The residue was purified by FCC on silica gel eluting with PE/EtOAc (8:1) to afford 12.8 g (88%) of N-(4-cyano-2-fluorophenyl)-1-(triisopropylsilyl) pyrrole-3-sulfonamide. LCMS (ES−, m/z) [M−1]⁻=420.1. ¹H NMR (400 MHz, DMSO-d6) δ 10.43 (s, 1H), 7.79 (m, 1H), 7.65-7.47 (m, 2H), 7.30-7.21 (m, 1H), 6.98-6.86 (m, 1H), 6.49 (dd, J=2.8, 1.3 Hz, 1H), 1.65-1.19 (m, 3H), 1.05-0.87 (m, 18H).

Step 2: To a stirred solution of N-(4-cyano-2-fluorophenyl)-1-(triisopropylsilyl) pyrrole-3-sulfonamide (12.8 g, 30.36 mmol) in THF (150 mL) was added NBS (5.4 g, 30.36 mmol) at −78° C. The mixture was stirred at −78° C. for 2 h and then was warmed to RT and stirred for additional 2 h. The resulting mixture was dissolved with EtOAc (500 mL) and the organic phase was washed with water (500 mL×3), followed by brine (500 mL), dried over anhydrous Na₂SO₄, filtered and concentrated under reduced pressure. The residue was purified by FCC on silica gel eluting with PE/EtOAc (10:1) to afford 9.2 g (60%) of 4-bromo-N-(4-cyano-2-fluorophenyl)-1-(triisopropylsilyl) pyrrole-3-sulfonamide. LCMS (ES−, m/z) [M−H]⁻=498.0, 500.0. ¹H NMR (400 MHz, CHCl₃) δ 7.62 (m, 1H), 7.39-7.32 (m, 2H), 7.31 (dd, J=3.0, 1.5 Hz, 1H), 7.29-7.23 (m, 1H), 6.72 (d, J=2.5 Hz, 1H), 1.46-1.32 (m, 3H), 1.05 (dd, J=16.3, 7.5 Hz, 18H).

Step 3: To a stirred solution of 4-bromo-N-(4-cyano-2-fluorophenyl)-1-(triisopropylsilyl) pyrrole-3-sulfonamide (9.2 g, 18.38 mmol) in THF (90 mL) was added TBAF (9.61 g, 36.76 mmol) in small portions at RT under N₂ atmosphere. The RM was stirred for 3 h at RT under N₂ atmosphere. The resulting mixture was concentrated under reduced pressure. The resulting mixture was extracted with EtOAc (3×500 mL). The combined organic layers were washed with brine (3×200 mL), dried over anhydrous Na₂SO₄, filtered and concentrated under reduced pressure. The residue was purified by FCC on silica gel eluting with PE/EtOAc (2:1) to afford 4.8 g (75%) of 4-bromo-N-(4-cyano-2-fluorophenyl)-1H-pyrrole-3-sulfonamide. LCMS (ES−, m/z) [M−H]⁻=341.8, 343.8. ¹H NMR (400 MHz, DMSO-d6) δ 11.98 (s, 1H), 10.69 (s, 1H), 7.82 (dd, J=10.6, 1.8 Hz, 1H), 7.62-7.55 (m, 1H), 7.55-7.47 (m, 2H), 7.08 (m, 1H).

Step 4: To a stirred solution of 4-bromo-N-(4-cyano-2-fluorophenyl)-1H-pyrrole-3-sulfonamide (4.8 g, 13.95 mmol) and Et₃N (3.53 g, 34.87 mmol) in DCM (70 mL) was added TsCl (2.66 g, 13.95 mmol) in small portions at RT under N₂ atmosphere and the resulting mixture was stirred for 16 h under nitrogen atmosphere. The solvent was evaporated under reduced pressure. The residue was purified by FCC on silica gel eluting with DCM/PE (1:1) to afford 3.8 g (54%) of 4-bromo-N-(4-cyano-2-fluorophenyl)-1-(4-methyl benzenesulfonyl) pyrrole-3-sulfonamide. LCMS (ES−, m/z) [M−H]⁻=495.9, 497.9. ¹H NMR (400 MHz, DMSO-d6) δ 11.09 (s, 1H), 8.06 (d, J=2.7 Hz, 1H), 8.00-7.94 (m, 2H), 7.84 (d, J=2.7 Hz, 1H), 7.77 (dd, J=10.6, 1.9 Hz, 1H), 7.56-7.47 (m, 3H), 7.46 (m, 1H), 2.44 (s, 3H).

Step 5: To a stirred mixture of 4-bromo-N-(4-cyano-2-fluorophenyl)-1-(4-methylbenzenesulfonyl) pyrrole-3-sulfonamide (3.8 g, 7.62 mmol) and DIPEA (1.18 g, 9.15 mmol) in DCM (40 mL) was added chloromethyl methyl ether (740 mg, 9.15 mmol) dropwise at 0° C. under N₂ atmosphere and the resulting mixture was stirred for 16 h at RT. The mixture was concentrated under reduced pressure. The residue was purified by FCC on silica gel eluting with PE/EtOAc (5:1) to afford 2.8 g (67%) of 4-bromo-N-(4-cyano-2-fluorophenyl)-N-(methoxymethyl)-1-(4-methyl benzenesulfonyl) pyrrole-3-sulfonamide. LCMS (ES−, m/z) [M−H]⁻=539.9, 541.9. ¹H NMR (400 MHz, DMSO-d6) δ 8.03-7.95 (m, 2H), 7.94 (d, J=2.7 Hz, 1H), 7.90-7.83 (m, 2H), 7.66 (dd, J=8.2, 1.8 Hz, 1H), 7.53 (d, J=8.2 Hz, 2H), 7.44 (m, 1H), 5.05 (s, 2H), 3.29 (s, 3H), 2.45 (s, 3H).

Step 6: To a stirred solution of 4-bromo-N-(4-cyano-2-fluorophenyl)-N-(methoxymethyl)-1-(4-methylbenzenesulfonyl) pyrrole-3-sulfonamide (2 g, 3.68 mmol) in DME (20 mL) was added i-PrMgCl (398 mg, 3.86 mmol) dropwise at −20° C. under N₂ atmosphere. The RM was stirred for 3 h at −20° C. under N₂ atmosphere and then was treated dropwise with a mixture of phenylacetaldehyde (444 mg, 3.68 mmol) and THF (10 mL) over 15 min. The resulting mixture was stirred for additional 8 h at RT. The RM was quenched by saturated aqueous NH₄Cl (20 mL) at RT. The resulting mixture was extracted with EtOAc (3×100 mL). The combined organic layers were washed with brine (3×50 mL), dried over anhydrous Na₂SO₄, filtered and concentrated under reduced pressure. The residue was purified by FCC on silica gel eluting with PE/EtOAc (4:1) to afford 760 mg (35%) of N-(4-cyano-2-fluorophenyl)-4-(1-hydroxy-2-phenylethyl)-N-(methoxymethyl)-1-tosyl-1H-pyrrole-3-sulfonamide. LCMS (ES−, m/z) [M−H]⁻=582.3.

Step 7. To a stirred mixture of N-(4-cyano-2-fluorophenyl)-4-(1-hydroxy-2-phenylethyl)-N-(methoxymethyl)-1-tosyl-1H-pyrrole-3-sulfonamide (700 mg, 1.20 mmol) in THF (6 mL) was added aq. HCl (218 mg, 5.99 mmol) dropwise at RT under N₂ atmosphere. The RM was stirred for 16 h. The resulting mixture was concentrated under reduced pressure. To the above mixture was added LiOH (143 mg, 5.99 mmol) in MeOH (10 mL) and H₂O (5 mL) and the resulting mixture was stirred for additional 1 h at RT. The mixture was concentrated under reduced pressure and was acidified to pH 7 with aqueous HCl. The resulting mixture was extracted with EtOAc (3×150 mL). The combined organic layers were washed with brine (3×50 mL), dried over anhydrous Na₂SO₄, filtered and concentrated under reduced pressure to afford 320 mg (69%) of crude N-(4-cyano-2-fluorophenyl)-4-(1-hydroxy-2-phenylethyl)-1H-pyrrole-3-sulfonamide. LCMS (ES−, m/z) [M−H]⁻=384.0.

Step 8: To a stirred solution of N-(4-cyano-2-fluorophenyl)-4-(1-hydroxy-2-phenylethyl)-1H-pyrrole-3-sulfonamide (320 mg, 0.832 mmol) and triethylsilane (483 mg, 4.15 mmol) in DCE (6 mL) was added TEA (570 mg, 4.98 mmol) dropwise at RT under N₂ atmosphere. The resulting mixture was stirred for 6 h at 70° C. under N₂ atmosphere. The resulting mixture was concentrated under vacuum. The residue was purified by preparative HPLC on a Sunfire prep C18 Column (30×150 mm, 5 μm); Mobile Phase A: Water(0.1% FA), Mobile Phase B: MeCN; Flow rate: 60 mL/min; Gradient: 48% B to 61% B in 9 min, 61% B. The purification afforded 120 mg (37%) of N-(4-cyano-2-fluorophenyl)-4-(2-phenylethyl)-1H-pyrrole-3-sulfonamide (Cpd 623).

The following compounds were prepared in a similar manner (use of appropriate reagents and purification methods (including chiral HPLC or chiral SFC) known to the person skilled in the art) as described for Cpd 623: Cpd 616; Cpd 624 and Cpd 625.

### Synthesis of N-(4-cyano-2,5-difluorophenyl)-4-((3-fluorophenyl)methyl-d2)-1H-pyrrole-3-sulfonamide (Cpd 643)

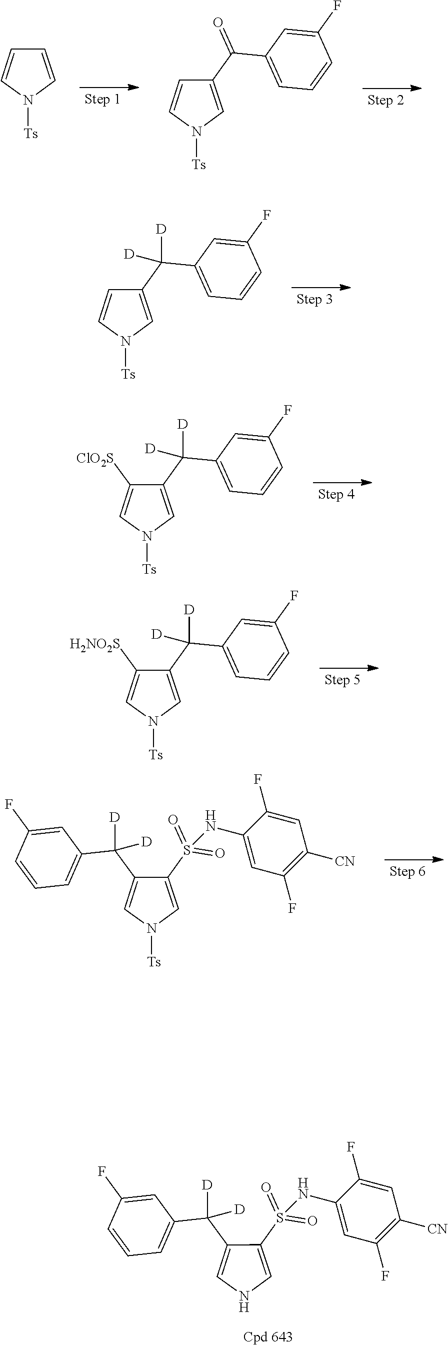

Step 1: To a stirred solution of AlCl₃ (3.6 g, 27.087 mmol) in DCE (35.0 mL) was added 3-fluorobenzoyl chloride (3.019 mL, 24.830 mmol) dropwise at 0° C. followed by a solution of 1-tosyl-1H-pyrrole (5.0 g, 22.572 mmol) in DCE (5.0 mL). The resulting mixture was stirred at RT for 2 h. Upon completion, reaction was concentrated under reduced pressure and the residue was diluted with EtOAc. It was washed with water, brine and dried over anhydrous Na₂SO₄, filtered and concentrated under reduced pressure. The residue was then purified by FCC over silica gel using a gradient of EtOAc (0 to 40%) in hexane and further washed with saturated aq. NaHCO₃ to afford 3.2 g (41%) of (3-fluorophenyl)(1-tosyl-1H-pyrrol-3-yl)methanone. ¹H NMR (400 MHz, DMSO): δ ppm 8.00 (d, 2H), 7.92 (br s, 1H), 7.62-7.58 (m, 2H), 7.55-7.50 (m, 3H), 7.47 (d, 2H), 6.77-6.76 (m, 1H), 2.42 (s, 3H).

Step 2: To a stirred solution of AlCl₃ (1.55 g, 11.66 mmol) and LiAlD₄ (441 mg, 10.49 mmol) in diethylether (20.0 mL) was added (3-fluorophenyl)(1-tosyl-1H-pyrrol-3-yl)methanone (2 g, 5.83 mmol) in diethylether (10.0 mL) at −20° C. and the reaction mixture was stirred for 15 minutes. After that it was refluxed for 2 hours. After completion, the RM was quenched by Fischer work up and filtered through a small bed of celite. Filtrate was concentrated under reduced pressure and crude thus obtained was purified by FCC over silica gel using a gradient of EtOAc (5 to 10%) in hexane to afford 650 mg (34%) of 3-((3-fluorophenyl)methyl-d2)-1-tosyl-1H-pyrrole. LC-MS (ES+, m/z) [M+H]⁺=332.2.

Step 3: Chlorosulfonic acid (0.573 mL, 8.61 mmol) was added to a precooled solution of 3-((3-fluorophenyl)methyl-d2)-1-tosyl-1H-pyrrole (570 mg, 1.722 mmol) in dry MeCN (10.0 mL) at 0° C. under N₂ atmosphere. RM was then heated at 70° C. for 16 h. After completion of the reaction, it was evaporated under reduced pressure and quenched with cold water. After that it was extracted with DCM twice and the combined organic layer was then dried over Na₂SO₄, filtered and evaporated under reduced pressure to afford 450 mg of crude 1-(dioxo(p-tolyl)-17-sulfanyl)-4-((3-fluorophenyl)methyl-d2)-1H-pyrrole-3-sulfonyl chloride which was directly used for next step without further purification.

Step 4: To a stirred solution of 1-(dioxo(p-tolyl)-17-sulfanyl)-4-((3-fluorophenyl)methyl-d2)-1H-pyrrole-3-sulfonyl chloride (450 mg, crude) in THF (5.0 mL) was added aq. NH₃ in THF and the reaction mixture was stirred at RT for 3 h. RM was concentrated under reduced pressure and the residue was purified using by FCC over silica gel using a gradient of EtOAc (20 to 60% ethyl) in hexane to afford 250 mg (58%) of 4-((3-fluorophenyl)methyl-d2)-1-tosyl-1H-pyrrole-3-sulfonamide. LCMS (ES−, m/z) [M−H]⁻=409.1.

Step 5: 4-((3-fluorophenyl)methyl-d2)-1-tosyl-1H-pyrrole-3-sulfonamide (250 mg, 0.61 mmol) was taken in MeCN (15.0 mL) and was degassed under argon atmosphere, followed by the addition of 4-bromo-2,5-difluorobenzonitrile (133 mg, 0.61 mmol) and K₂CO₃ (210 mg, 0.524 mmol) and was further degassed for sometimes and then CuI (41 mg, 0.213 mmol) and trans-N,N′-Dimethyl-cyclohexane-1,2-diamine (68.5 mg, 0.48 mmol) were added to the RM. The RM was then heated at 100° C. for 16 h. After completion, the RM was evaporated under reduced pressure and purified by FCC over silica gel using a gradient of EtOAc (30 to 50%) in hexane to afford 270 mg (82%) of N-(4-cyano-2,5-difluorophenyl)-4-((3-fluorophenyl)methyl-d2)-1-tosyl-1H-pyrrole-3-sulfonamide. LCMS (ES−, m/z) [M−H]⁻=545.9. ¹H NMR (400 MHz, DMSO): δ ppm 11.25 (br s, 1H), 8.06 (br, 1H), 7.88 (d, 2H), 7.72-7.71 (m, 1H), 7.41 (d, 2H), 7.26-7.21 (m, 2H), 7.14 (s, 1H), 6.98-6.92 (m, 2H), 6.84 (d, 1H), 2.40 (s, 3H);

Step 6: To a stirred solution of N-(4-cyano-2,5-difluorophenyl)-4-((3-fluorophenyl)methyl-d2)-1-tosyl-1H-pyrrole-3-sulfonamide (270 mg, 0.429 mmol) in THF/MeOH/H₂O (2:1:1, 24.0 mL) at 0° C. was added LiOH·H₂O (103 mg, 2.459 mmol) and the RM was stirred for 4 h at RT. After completion of the reaction, RM was concentrated under reduced pressure, pH was made acidic and extracted with EtOAc. Organic phase was washed with brine, dried over anhydrous Na₂SO₄, filtered and concentrated under reduced pressure. The residue was purified by FCC over silica gel using a gradient of EtOAc (0 to 50%) in hexane to afford 75 mg (39%) of N-(4-cyano-2,5-difluorophenyl)-4-((3-fluorophenyl) methyl-d2)-1H-pyrrole-3-sulfonamide (Cpd 643). LCMS (ES−, m/z) [M−H]⁻=392.16. ¹H NMR (400 MHz, DMSO): δ ppm 11.60 (s, 1H), 10.92 (s, 1H), 7.86-7.82 (m, 1H), 7.57-7.56 (m, 1H), 7.32-7.20 (m, 2H), 6.98-6.89 (m, 3H), 6.60 (s, 1H).

### Synthesis of N-(4-cyano-2-fluorophenyl)-4-phenyl-1H-pyrrole-3-sulfonamide (Cpd 009)

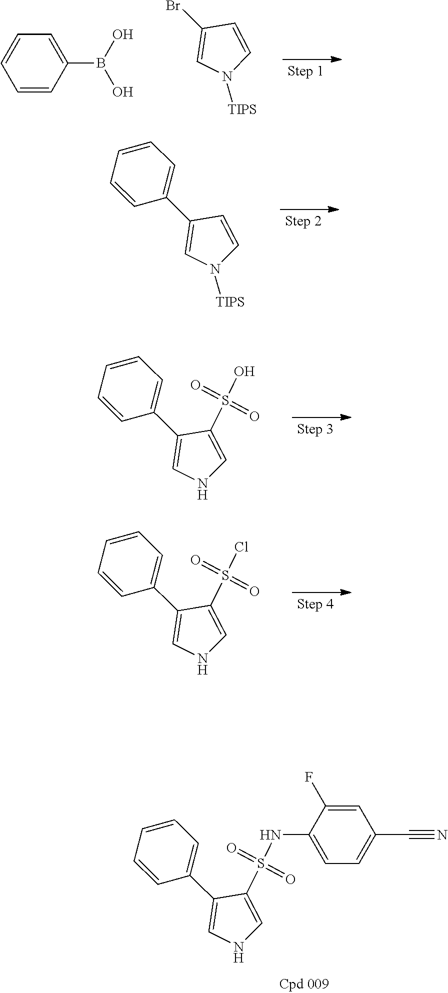

Step 1: To a mixture of 3-bromo-1-(triisopropylsilyl)pyrrole (4.50 g, 14.88 mmol) and phenyl boronic acid (3.63 g, 29.77 mmol) in Toluene (40 ml) and H₂O (2 mL) were added Na₂CO₃ (4.73 g, 44.65 mmol) and Pd(dppf)Cl₂ (0.22 g, 0.30 mmol) at RT. The RM was stirred for 8 h at 100° C. under N₂ atmosphere. The RM was concentrated under reduced pressure. The residue was purified by FCC over silica gel using as eluent of PE/EtOAc (50/1). The residue was purified again by R^{p} flash chromatography on C18 gel using a gradient of MeCN (60 to 100%) in water to afford 3.38 g (76%) of 3-phenyl-1-(triisopropylsilyl)pyrrole. ¹H NMR (400 MHz, DMSO-d6) δ 7.61-7.53 (m, 2H), 7.35-7.23 (m, 3H), 7.16-7.08 (m, 1H), 6.86 (t, 1H), 6.62 (dd, 1H), 1.61-1.41 (m, 3H), 1.12-1.02 (m, 18H).

Step 2: To a solution of 3-phenyl-1-(triisopropylsilyl)pyrrole (2.00 g, 6.68 mmol) and Py·SO₃ (1.59 g, 9.99 mmol) in MeCN (20 mL) was stirred for 8 h at 120° C. The RM was concentrated under reduced pressure. The residue was dissolved in water (50 mL) and washed with CHCl₃ (50 mL×3). The aqueous phase was concentrated under reduced pressure to afford 2.5 g of 4-phenyl-1H-pyrrole-3-sulfonic acid, which was used without further purification.

Step 3: To a solution of 4-phenyl-1H-pyrrole-3-sulfonic acid (2.50 g) in MeCN (20 mL) was added was added POCl₃ (3.43 g, 22.37 mmol) at 0° C. The RM was stirred for 4 h at 70° C. The RM was poured into ice-water and extracted with DCM (3×50 mL). The organic layers were combined, dried over Na₂SO₄, filtrated, and concentrated under reduced pressure to afford 1.2 g of 4-phenyl-1H-pyrrole-3-sulfonyl chloride, which was used without further purification.

Step 4: To a solution of 4-amino-3-fluorobenzonitrile (1.01 g, 7.45 mmol) and pyridine (3.93 g, 49.65 mmol) in MeCN (15 mL) was added 4-phenyl-1H-pyrrole-3-sulfonyl chloride (1.20 g) in MeCN (5 mL) dropwise at RT. The RM was stirred overnight at RT under nitrogen atmosphere. The RM was concentrated under reduced pressure. The residue was purified by preparative HPLC on a Xselect CSH Prep C18 OBD Column (19×150 mm, 5 μm); Mobile Phase A: Water (0.05% FA), Mobile Phase B: MeCN; Flow rate: 25 mL/min; Gradient: 33% to 50% of B in 8 min. The purification afforded 25.7 mg (1%) of N-(4-cyano-2-fluorophenyl)-4-phenyl-1H-pyrrole-3-sulfonamide (Cpd 009).

### Synthesis of N-(4-bromo-2,5-difluoro-phenyl)-4-(3-fluorophenyl)-1H-pyrrole-3-sulfonamide (Cpd 359)

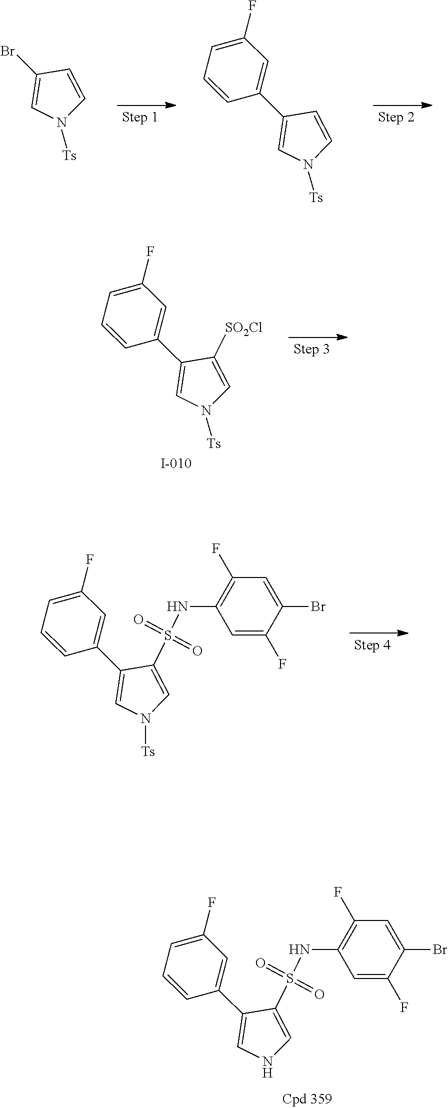 [SMILES: Cc1ccc(S(=O)(=O)n2ccc(Br)c2)cc1 ; Cc1ccc(S(=O)(=O)n2ccc(-c3cccc(F)c3)c2)cc1 ; Cc1ccc(S(=O)(=O)n2cc(-c3cccc(F)c3)c(S(=O)(=O)Cl)c2)cc1 ; Cc1ccc(S(=O)(=O)n2cc(-c3cccc(F)c3)c(S(=O)(=O)Nc3cc(F)c(Br)cc3F)c2)cc1 ; O=S(=O)(Nc1cc(F)c(Br)cc1F)c1c[nH]cc1-c1cccc(F)c1]

Step 1: To a solution 3-bromo-1-tosyl-1H-pyrrole (500.0 mg. 1.66 mmol) in MeOH (2 mL), toluene (2 mL) and water (2 ml), were added (3-fluorophenyl)boronic acid (279.68 mg, 2 mmol) and K₂CO₃ (574.7 mg, 4.2 mmol). The RM was degassed with argon for 15 minutes. Pd(dppf)Cl₂ (121.9 mg, 0.17 mmol) was then added to the RM. The RM was heated at 80° C. overnight. The RM was diluted with water and extracted with EtOAc. The organic layers were combined, washed with brine, dried over Na₂SO₄, filtered, and evaporated under reduced pressure. The residue was purified by FCC over silica gel using a gradient of EtOAc (0 to 5%) in hexane to afford 140 mg (27%) of 3-(3-fluorophenyl)-1-tosyl-1H-pyrrole. ¹H NMR (400 MHz, CDCl₃): δ ppm 7.78-7.76 (m, 2H), 7.40-7.39 (m, 1H), 7.31-7.27 (m, 2H), 7.26-7.24 (m, 2H), 7.20-7.18 (m, 1H), 7.16-7.12 (m, 1H), 6.94-6.88 (m, 1H), 6.56-6.55 (m, 1H), 2.39 (s, 3H).

Step 2: A solution of 3-(3-fluorophenyl)-1-tosyl-1H-pyrrole (90.0 mg, 0.28 mmol) in MeCN (5.0 mL) was cooled to 0° C. and then chlorosulfonic acid (0.095 mL, 1.43 mmol) was added. The RM was heated at 80° C. for 16 h. After cooling down to RT, the RM was concentrated under reduced pressure to afford 100 mg of 4-(3-fluorophenyl)-1-tosyl-1H-pyrrole-3-sulfonyl chloride (I-010); which was used without further purification.

Step 3: To a solution of 4-(3-fluorophenyl)-1-tosyl-1H-pyrrole-3-sulfonyl chloride (230 mg, 0.556 mmol) in MeCN (3.0 ml) was added 4-bromo-2,5-difluoroaniline (I-010) (115.6 mg, 0.56 mmol) at RT. Pyridine (0.112 ml, 1.39 mmol) was added to the RM. The RM was heated at 80° C. for 16 h. After cooling down to RT, the RM was concentrated under reduced pressure. The residue was purified by FCC over silica gel using a gradient of EtOAc (0 to 70%) in hexane to afford 130 mg (40%) of N-(4-bromo-2,5-difluorophenyl)-4-(3-fluorophenyl)-1-tosyl-1H-pyrrole-3-sulfonamide. ¹H NMR (400 MHz, CDCl₃): δ ppm 7.86-7.85 (m, 1H), 7.79-7.76 (m, 2H), 7.37-7.35 (m, 2H), 7.31-7.29 (m, 1H), 7.18-7.15 (m, 2H), 7.12-7.05 (m, 4H), 6.38 (br s, 1H), 2.45 (s, 3H).

Step 4: To a solution of N-(4-bromo-2,5-difluorophenyl)-4-(3-fluorophenyl)-1-tosyl-1H-pyrrole-3-sulfonamide (130 mg, 0.22 mmol) in (2:1) MeOH and Water (4.5 ml) was added LiOH·H₂O (46.6 mg, 1.11 mmol) at 0° C. The RM was stirred at RT for 1 h. The RM was diluted with water and extracted with EtOAc. The organic layers were combined, washed with brine solution, dried over Na₂SO₄, filtered, and concentrated under reduced pressure. The residue was purified by preparative HPLC on Xterra R^{p} 18 (250×19 mm, 10p) column, operating at flow rate of 16 mL/min. Mobile phase: A=20 mM NH₄HCO₃ in water, B=MeCN; Gradient Profile: Mobile phase initial composition of 70% A and 30% B, then 40% A and 60% B in 3 min, then to 20% A and 80% B in 22 min., then to 5% A and 95% B in 23 min., held this composition up to 25 min. The purification afforded 15 mg (16%) of N-(4-bromo-2,5-difluorophenyl)-4-(3-fluorophenyl)-1H-pyrrole-3-sulfonamide (Cpd 359).

The following compound was prepared in a similar manner (use of appropriate reagents and purification methods (including chiral HPLC or chiral SFC) known to the person skilled in the art) as described for Cpd 359: Cpd 364; Cpd 425; Cpd 428; Cpd 438; Cpd 460; Cpd 466 (from I-017); Cpd 469; Cpd 496; Cpd 497.

### Synthesis of N-[4-(difluoromethoxy)-2,5-difluoro-phenyl]-4-(3-fluorophenyl)-1H-pyrrole-3-sulfonamide (Cpd 360) from I-010

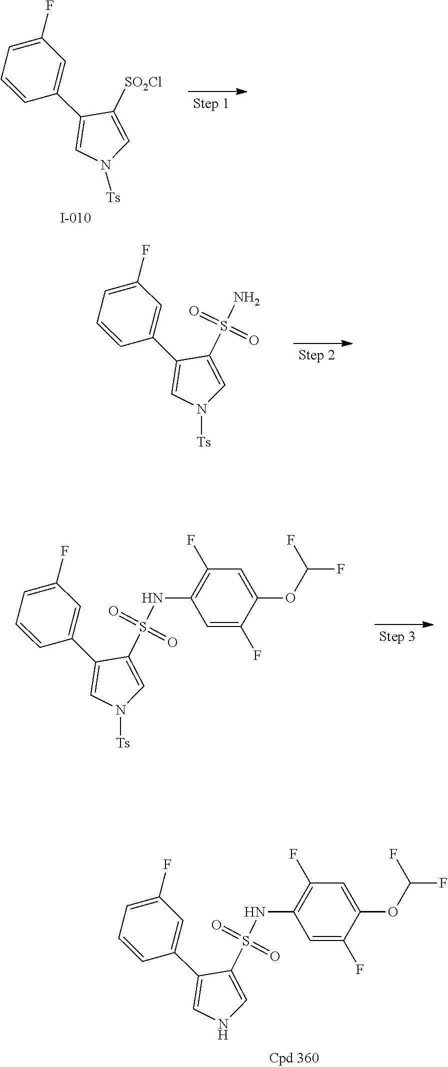 [SMILES: Cc1ccc(S(=O)(=O)n2cc(-c3cccc(F)c3)c(S(=O)(=O)Cl)c2)cc1 ; Cc1ccc(S(=O)(=O)n2cc(-c3cccc(F)c3)c(S(N)(=O)=O)c2)cc1 ; Cc1ccc(S(=O)(=O)n2cc(-c3cccc(F)c3)c(S(=O)(=O)Nc3cc(F)c(OC(F)F)cc3F)c2)cc1 ; O=S(=O)(Nc1cc(F)c(OC(F)F)cc1F)c1c[nH]cc1-c1cccc(F)c1]

Step 1: To a solution of 4-(3-fluorophenyl)-1-tosyl-1H-pyrrole-3-sulfonyl chloride (I-010) (500 mg, 1.21 mmol) in THF (3 ml) was added aq. NH₃ (0.241 ml, 6.0 mmol). The RM was stirred at RT for 16 h. The RM was concentrated under reduced pressure. The residue was washed with hexane and diethylether to afford 390 mg of 4-(3-fluorophenyl)-1-tosyl-1H-pyrrole-3-sulfonamide, which was used without further purification.

Step 2: To a mixture of 4-(3-fluorophenyl)-1-tosyl-1H-pyrrole-3-sulfonamide (380 mg, 0.96 mmol) and 1-bromo-4-(difluoromethoxy)-2,5-difluorobenzene (299.4 mg, 1.16 mmol) was added MeCN (5.0 mL) in a sealed tube. The RM was degassed under Argon for 15 minutes. K₂CO₃ (332.4 mg, 2.41 mmol), CuI (9.17 mg, 0.05 mmol), and trans-N,N′-Dimethyl-cyclohexane-1,2-diamine (68.5 mg, 0.48 mmol) were added to the RM. The RM was heated at 120° C. for 16 h. The RM was filtered through celite bed. Filtrate was then diluted with EtOAc and water. Organic layer was separated, dried over Na₂SO₄, filtrated, and concentrated under reduced pressure. The residue was purified by FCC over silica gel using a gradient of EtOAc (0 to 10%) in hexane to afford 130 mg (24%) of N-(4-(difluoromethoxy)-2,5-difluorophenyl)-4-(3-fluorophenyl)-1-tosyl-1H-pyrrole-3-sulfonamide.

Step 3: To a stirred solution of N-(4-(difluoromethoxy)-2,5-difluorophenyl)-4-(3-fluorophenyl)-1-tosyl-1H-pyrrole-3-sulfonamide (130.0 mg, 0.227 mmol) in MeOH/Water (2:1, 4.5 ml). was added LiOH·H₂O (47.6 mg, 1.14 mmol) at 0° C. The RM was stirred at RT for 1 h. The RM was diluted with water and extracted with EtOAc. The organic layers were combined, washed with brine, dried over Na₂SO₄, filtered, and concentrated under reduced pressure. The residue was purified by preparative HPLC on YMC Triart C18 (250×20 mm, 5p) column, operating at flow rate of 16 mL/min. Mobile phase: A=20 mM NH₄HCO₃ in water, B=MeCN; Gradient Profile: Mobile phase initial composition of 80% A and 20% B, then 55% A and 45% B in 2 min, then to 20% A and 80% B in 22 min., then to 5% A and 95% B in 23 min., held this composition up to 25 min. The purification afforded 30 mg (32%) of N-(4-(difluoromethoxy)-2,5-difluorophenyl)-4-(3-fluorophenyl)-1H-pyrrole-3-sulfonamide) (Cpd 360).

### Synthesis of N-(4-cyano-2-fluoro-phenyl)-4-(4-fluorophenyl)-1H-pyrrole-3-sulfonamide (Cpd 217)

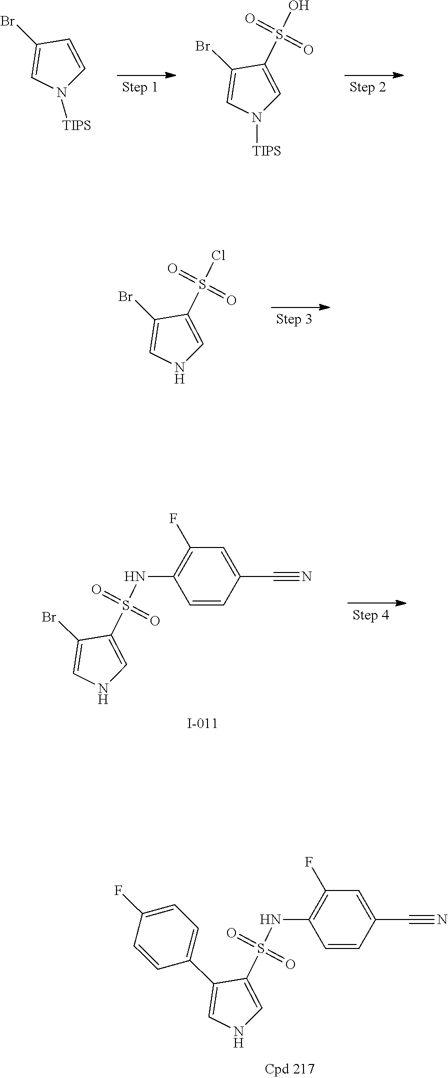

Step 1: To a solution of Chlorosulfonic acid (1.67 mL, 25.1 mmol) in DCM (50 mL) was slowly added 3-bromo-1-(triisopropylsilyl)-1H-pyrrole (6.89 g, 22.8 mmol) at 0° C. The RM was stirred for 1 h. The RM was concentrated to afford 6.59 g (76%) of 4-bromo-1-(triisopropylsilyl)-1H-pyrrole-3-sulfonic acid; which was used without further purification.

Step 2: To a solution of 4-bromo-1-(triisopropylsilyl)-1H-pyrrole-3-sulfonic acid (6.59 g, 17.2 mmol) in DCM (60 mL) was added oxalyl chloride (7.27 mL, 85.9 mmol) and DMF (5 drops) at 0° C. The RM was stirred for 3 h at 60° C. After completion, the RM was concentrated under reduced pressure, diluted with water, and extracted with EtOAc. The organic layers were combined, washed with brine, dried over Na₂SO₄, filtrated, and concentrated under reduced pressure to afford 4 g (95%) of 4-bromo-1H-pyrrole-3-sulfonyl chloride; which was used without further purification.

Step 3: To a solution of 4-bromo-1H-pyrrole-3-sulfonyl chloride (0.150 g, 0.61 mmol), in MeCN (3 mL), was added 4-amino-3-fluorobenzonitrile (0.067 g, 0.49 mmol) and pyridine (0.13 mL, 1.54 mmol). The RM was heated at 90° C. for 16 h. After completion, the RM was concentrated, diluted with water and extracted with EtOAc. The organic layers were combined, washed with brine, dried over Na₂SO₄, filtrated, and concentrated under reduced pressure. The residue was purified by FCC over silica gel using a gradient of EtOAc (0 to 60%) in DCM. The residue was further purified by preparative HPLC on YMC Triart C18 (250×20 mm, 5p) column, operating at flow rate of 16 mL/min. Mobile phase: A=20 mM NH₄HCO₃ in water, B=MeCN; Gradient Profile: Mobile phase initial composition of 80% A and 20% B, then 70% A and 30% B in 3 min, then to 40% A and 60% B in 22 min., then to 5% A and 95% B in 23 min., held this composition up to 25 min. The purification afforded 0.03 g (14%) of 4-bromo-N-(4-cyano-2-fluorophenyl)-1H-pyrrole-3-sulfonamide (I-011). ¹H NMR (400 MHz, DMSO-d₆): δ ppm 11.96 (brs, 1H), 10.67 (s, 1H), 7.80 (d, 1H), 7.58-7.47 (m, 3H), 7.06 (s, 1H).

Step 4: To a solution of 4-bromo-N-(4-cyano-2-fluorophenyl)-1H-pyrrole-3-sulfonamide (I-011) (150 mg, 0.436 mmol) in t-Amyl alcohol (5 ml) was added (4-fluorophenyl)boronic acid (91 mg, 0.66 mmol). A solution of K₂CO₃ (181.6 mg, 1.32 mmol) in water (0.5 ml) was added. The RM was degassed with argon followed by the addition of Pd(amphos)Cl₂ (31 mg, 0.044 mmol). The RM was stirred at 80° C. for 16 h. The RM was concentrated under reduced pressure. The residue was purified by FCC over silica gel using a gradient of EtOAc (0 to 10%) in DCM. The residue was purified again by preparative HPLC on Xterra R^{p} 18 (250×19 mm, 10p) column, operating at flow rate of 16 mL/min. Mobile phase: A=20 mM NH₄HCO₃ in water, B=MeCN; Gradient Profile: Mobile phase initial composition of 80% A and 20% B, then 50% A and 50% B in 3 min, then to 30% A and 70% B in 22 min., then to 5% A and 95% B in 23 min., held this composition up to 25 min. The purification afforded 17 mg (11%) of N-(4-cyano-2-fluorophenyl)-4-(4-fluorophenyl)-1H-pyrrole-3-sulfonamide (Cpd 217).

The following compounds were prepared in a similar manner (use of appropriate reagents and purification methods (including chiral HPLC or chiral SFC) known to the person skilled in the art) as described for Cpd 217: Cpd 218; Cpd 219; Cpd 220; Cpd 221; Cpd 222; Cpd 223; Cpd 228; Cpd 230; Cpd 231; Cpd 381; Cpd 395; Cpd 398; Cpd 400; Cpd 401; Cpd 402; Cpd 420.

### Synthesis of N-(4-cyano-2-fluoro-phenyl)-4-(cyclopentenyl-1-yl)-1H-pyrrole-3-sulfonamide (Cpd 238) and N-(4-cyano-2-fluoro-phenyl)-4-cyclopentyl-1H-pyrrole-3-sulfonamide (Cpd 242) from I-011

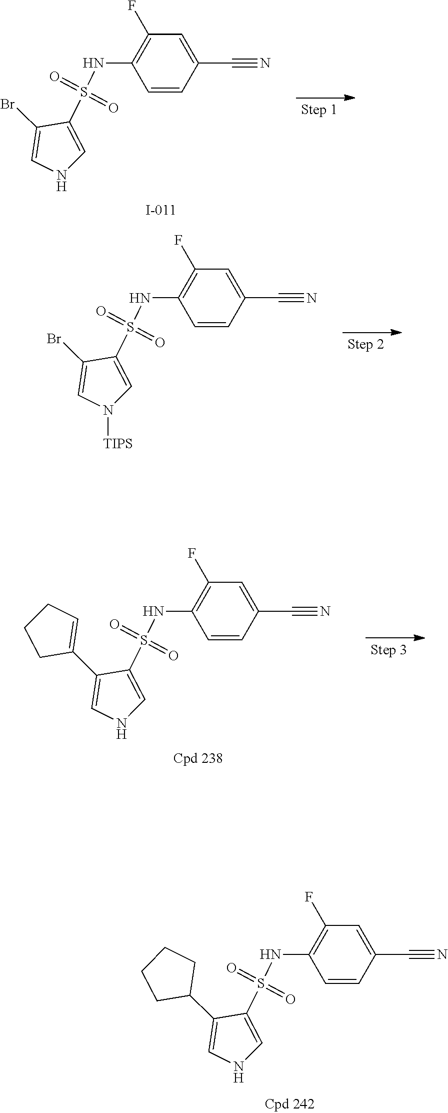

Step 1: To a solution of 4-bromo-N-(4-cyano-2-fluorophenyl)-1H-pyrrole-3-sulfonamide (I-011) (700 mg, 2.0 mmol) in THF (15 ml) was added NaH (60% in oil) (202 mg, 5.1 mmol) portion wise at 0° C. The RM was stirred for 0.5 h. Then TIPSCl (0.865 ml, 4.1 mmol) was added dropwise to the RM at RT. The RM was stirred at RT for 2 h. The RM was quenched with ice-cold water. The solution was extracted with EtOAc. The organic layers were combined, washed with brine, dried over Na₂SO₄, filtrated, and concentrated under reduced pressure. The residue was purified by FCC over silica gel using a gradient of EtOAc (0 to 40%) in hexane to afford 500 mg (49%) of 4-bromo-N-(4-cyano-2-fluorophenyl)-1-(triisopropylsilyl)-1H-pyrrole-3-sulfonamide.

Step 2: To a solution of 4-bromo-N-(4-cyano-2-fluorophenyl)-1-(triisopropylsilyl)-1H-pyrrole-3-sulfonamide (300 mg, 0.6 mmol) in t-Amyl alcohol (10 ml) was added 2-(cyclopent-1-en-1-yl)-4,4,5,5-tetramethyl-1,3,2-dioxaborolane (232.6 mg, 1.2 mmol). A solution of K₂CO₃ (248 mg, 1.8 mmol) in water (2 ml) was added to the RM. The RM was degassed with argon followed by the addition of Pd(amphos)Cl₂ (42 mg, 0.06 mmol). The RM was stirred at 80° C. for 16 h. The RM was concentrated under reduced pressure. The residue was purified by FCC over silica gel using a gradient of EtOAc (0 to 10%) in DCM. The residue was further purified by preparative HPLC on a YMC Actus Triart C18 (250×20 mm, 5p) column, operating at flow rate of 16 mL/min. Mobile phase: A=20 mM NH₄HCO₃ in water, B=MeCN; Gradient Profile: Mobile phase initial composition of 80% A and 20% B, then 60% A and 40% B in 3 min, then to 10% A and 90% B in 22 min., then to 5% A and 95% B in 23 min., held this composition up to 26 min. The purification afforded 198 mg (99%) of N-(4-cyano-2-fluorophenyl)-4-(cyclopent-1-en-1-yl)-1H-pyrrole-3-sulfonamide (Cpd 238).

Step 3: To a solution N-(4-cyano-2-fluorophenyl)-4-(cyclopent-1-en-1-yl)-1H-pyrrole-3-sulfonamide (Cpd 238) (0.02 g, 0.04 mmol) in MeOH (1 mL) and THF (2 mL) was added 10% Pd/C (50% moist) (0.015 g, 0.08 mmol) at RT and stirred for 16 h. The RM was filtered over celite bed. Filtrate was concentrated under reduced pressure. The residue was purified by preparative HPLC on a YMC Actus Triart C18 (250×20 mm, 5p) column, operating at flow rate of 16 mL/min. Mobile phase: A=20 mM NH₄HCO₃ in water, B=MeCN; Gradient Profile: Mobile phase initial composition of 70% A and 30% B, then 65% A and 35% B in 3 min, then to 30% A and 70% B in 20 min., then to 5% A and 95% B in 21 min., held this composition up to 23 min. The purification afforded 0.01 g (75%) of N-(4-cyano-2-fluorophenyl)-4-cyclopentyl-1H-pyrrole-3-sulfonamide (Cpd 242).

### Synthesis of N-(4-cyano-2-fluoro-phenyl)-4-(2-pyridyl)-1H-pyrrole-3-sulfonamide (Cpd 229) from I-011

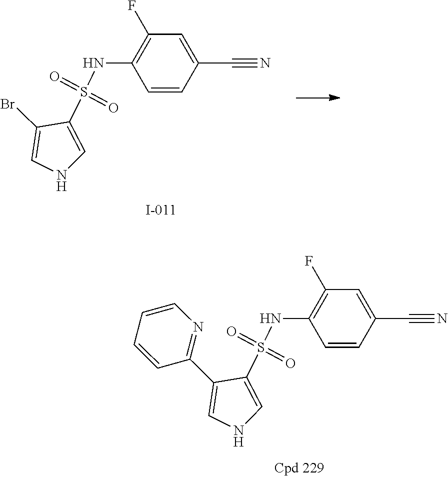 [SMILES: N#Cc1ccc(NS(=O)(=O)c2c[nH]cc2Br)c(F)c1 ; N#Cc1ccc(NS(=O)(=O)c2c[nH]cc2-c2ccccn2)c(F)c1]

A solution of 4-bromo-N-(4-cyano-2-fluorophenyl)-1-(triisopropylsilyl)-1H-pyrrole-3-sulfonamide (I-011) (0.2 g, 0.4 mmol) and 2-(tributylstannyl)pyridine (0.747 mL, 2.33 mmol) in dioxane (18 mL) was degassed with argon followed by the addition of Pd(PPh₃)₄ (0.034 g, 0.05 mmol). The RM was heated at 80° C. for 16 h in sealed tube. The RM was concentrated under reduced pressure. The residue was purified by FCC over silica gel using a gradient of EtOAc (0 to 50%) in hexane. The residue was triturated with pentane to afford 20 mg (15%) of N-(4-cyano-2-fluorophenyl)-4-(pyridin-2-yl)-1H-pyrrole-3-sulfonamide.

### Synthesis of N-(4-cyano-2-fluorophenyl)-4-(pyridin-3-yl)-1H-pyrrole-3-sulfonamide (Cpd 437)

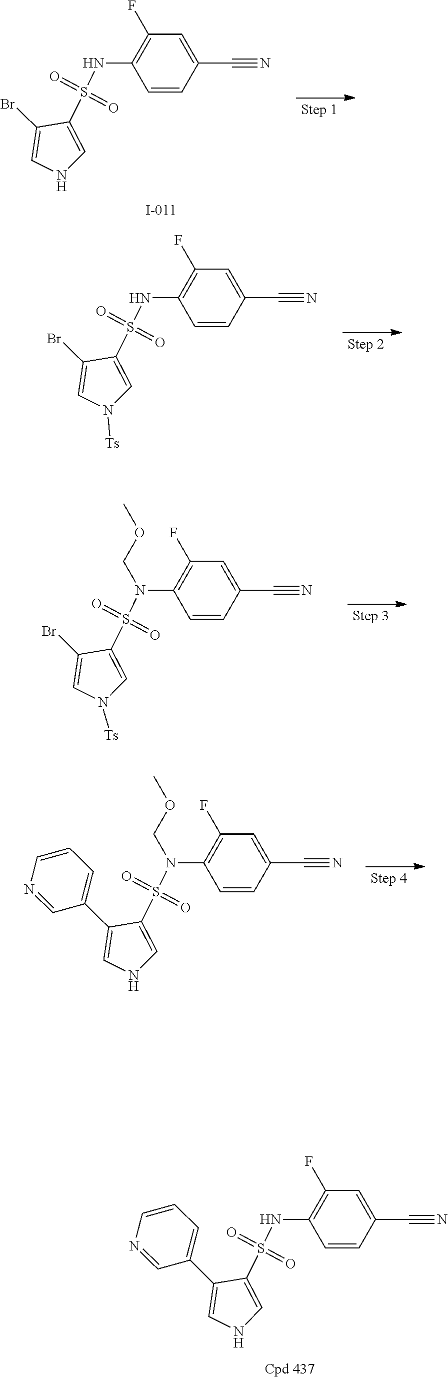 [SMILES: N#Cc1ccc(NS(=O)(=O)c2c[nH]cc2Br)c(F)c1 ; Cc1ccc(S(=O)(=O)n2cc(Br)c(S(=O)(=O)Nc3ccc(C#N)cc3F)c2)cc1 ; COCN(c1ccc(C#N)cc1F)S(=O)(=O)c1cn(S(=O)(=O)c2ccc(C)cc2)cc1Br ; COCN(c1ccc(C#N)cc1F)S(=O)(=O)c1c[nH]cc1-c1cccnc1 ; N#Cc1ccc(NS(=O)(=O)c2c[nH]cc2-c2cccnc2)c(F)c1]

Step 1: To a stirred mixture of 4-bromo-N-(4-cyano-2-fluorophenyl)-1-(triisopropylsilyl)-1H-pyrrole-3-sulfonamide (I-011) in DCM (10 mL) was added Et₃N (0.5 mL, 3.627 mmol) and the RM was stirred for 5 min. Then TsCl (276 mg, 1.451 mmol) was added and the RM was stirred at RT for 16 hours. After completion, RM was evaporated under reduced pressure and partitioned between EtOAc and water. Organic phase was separated, washed with brine, dried over anhydrous Na₂SO₄, filtered and concentrated. The residue was purified by FCC over silica gel using a gradient of EtOAc (0 to 50%) in hexane to afford 370 mg (51%) of 4-bromo-N-(4-cyano-2-fluorophenyl)-1-tosyl-1H-pyrrole-3-sulfonamide. LC-MS (Method-A:): Rt=2.93 min, (ES+H, m/z) [M−H]=496.0. ¹H NMR (400 MHz, DMSO-d6): δ ppm 11.07 (br s, 1H), 8.05-8.04 (m, 1H), 7.96 (d, J=8.36 Hz, 2H), 7.83-7.82 (m, 1H), 7.78-7.75 (m, 1H), 7.56-7.42 (m, 4H), 2.43 (s, 3H).

Step 2: To a stirred mixture of 4-bromo-N-(4-cyano-2-fluorophenyl)-1-tosyl-1H-pyrrole-3-sulfonamide (300 mg, 0.601 mmol) in dry DCM (10 mL), was added DIPEA (0.126 mL, 0.721 mmol) at 0° C. and RM was stirred for 20 min. MOMCl (0.055 mL, 0.721 mmol) was added dropwise to the reaction mixture at 0° C. Reaction was then stirred for 16 h at RT. After completion, volatiles were removed under reduced pressure. The residue was purified by FCC over silica gel using a gradient of EtOAc (0 to 20%) in hexane to afford 200 mg (61%) of 4-bromo-N-(4-cyano-2-fluorophenyl)-N-(methoxymethyl)-1-tosyl-1H-pyrrole-3-sulfonamide. LCMS (ES+, m/z) [M+H]+=539.9, 541.9. ¹H NMR (400 MHz, DMSO-d6): δ ppm 7.96 (d, J=8.32 Hz, 2H), 7.93-7.92 (m, 1H), 7.86-7.84 (m, 2H), 7.66-7.64 (m, 1H), 7.52 (d, J=8.24 Hz, 2H), 7.43 (t, J=7.9 Hz, 1H), 5.03 (s, 2H), 3.27 (s, 3H), 2.45 (s, 3H).

Step 3: To a stirred degassed solution of 4-bromo-N-(4-cyano-2-fluorophenyl)-N-(methoxymethyl)-1-tosyl-1H-pyrrole-3-sulfonamide (490 mg, 0.903 mmol) in 1,4-dioxane (4.0 ml) was added pyridin-3-ylboronic acid (144 mg, 1.174 mmol). A solution of K₂CO₃ (375 mg, 2.71 mmol) in water (1.5 ml) was added to the reaction mixture and resulting mixture was degassed with argon. Pd(dppf)Cl₂ (66 mg, 0.09 mmol) was then added to the reaction mixture under inert atmosphere. RM was then stirred at 80° C. for 16 h. After completion, the RM was concentrated under reduced pressure to afford crude material. Crude was then purified by preparative TLC (eluting with 100% ethyl acetate) to afford 270 mg (77%) of N-(4-cyano-2-fluorophenyl)-N-(methoxymethyl)-4-(pyridin-3-yl)-1H-pyrrole-3-sulfonamide. LCMS (ES+, m/z) [M+H]⁺=387.2. H NMR (400 MHz, DMSO-d6): δ ppm 11.98 (s, 1H), 8.58-8.57 (m, 1H), 8.43-8.42 (m, 1H), 7.83-7.78 (m, 2H), 7.54-7.51 (m, 1H), 7.44-7.43 (m, 1H), 7.30-7.27 (m, 1H), 7.24-7.18 (m, 2H), 4.65 (s, 2H), 3.15 (s, 3H).

Step 4: To a stirred solution of N-(4-cyano-2-fluorophenyl)-N-(methoxymethyl)-4-(pyridin-3-yl)-1H-pyrrole-3-sulfonamide (270 mg, 0.699 mmol) in MeOH (8 mL) was added a solution of oxalic acid (567 mg, 6.294 mmol) in H₂O (4 mL). The resulting solution was refluxed for 16 hours. Upon completion (monitored by LCMS), reaction mixture was concentrated under reduced pressure and crude was then extracted by ethyl acetate and washed several times with water. The combine organic solution was then concentrated under reduced pressure to get crude material. Crude was then purified by R^{p} preparative HPLC on a YMC-Actus Triart C18 column (20×250 mm, 5 μm) operating with a flow rate of 16 mL/min; Mobile Phase A: 20 mM NH₄HCO₃ in water; Mobile Phase B: MeCN; Gradient profile: 20% B for 5 min, then to 60% in 25 min and to 95% in 1 minute, held for 2 min, then returned to initial composition in 1 min and held for 2 min. The purification afforded 56 mg (23%) of N-(4-cyano-2-fluorophenyl)-4-(pyridin-3-yl)-1H-pyrrole-3-sulfonamide (Cpd 437).

### Synthesis of N-(4-cyano-2-fluorophenyl)-5-methyl-4-phenyl-1H-pyrrole-3-sulfonamide (Cpd 461)

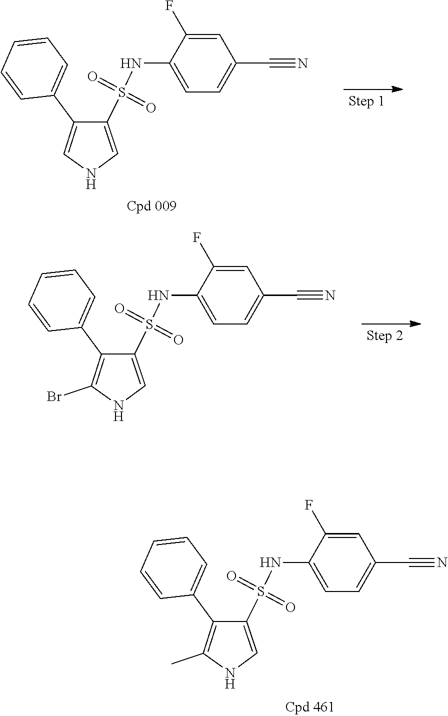 [SMILES: N#Cc1ccc(NS(=O)(=O)c2c[nH]cc2-c2ccccc2)c(F)c1 ; N#Cc1ccc(NS(=O)(=O)c2c[nH]c(Br)c2-c2ccccc2)c(F)c1 ; Cc1[nH]cc(S(=O)(=O)Nc2ccc(C#N)cc2F)c1-c1ccccc1]

Step 1. To a stirred solution of N-(4-cyano-2-fluorophenyl)-4-phenyl-1H-pyrrole-3-sulfonamide (Cpd 009) (380 mg, 1.113 mmol) in DMF (10.0 mL) was added NBS (356.59 mg, 1.002 mmol) at 0° C. and the mixture was stirred at RT for 16 h. Upon completion, RM was diluted with EtOAc, washed with water, brine, dried over Na₂SO₄, filtered and concentrated under reduced pressure. The residue was purified by FCC over silica gel using a gradient of EtOAc (20 to 40%) in hexane afford 220 mg (47%) of 5-bromo-N-(4-cyano-2-fluorophenyl)-4-phenyl-1H-pyrrole-3-sulfonamide. LCMS (ES−, m/z) [M−H]⁻=418.02, 420.0. ¹H NMR (400 MHz, DMSO-d6): δ ppm 12.64 (s, 1H), 10.35 (s, 1H), 7.73 (d, 1H), 7.60-7.59 (m, 1H), 7.47 (d, 1H), 7.35-7.30 (m, 4H), 7.22-7.20 (m, 2H);

Step 2: To a stirred solution of 5-bromo-N-(4-cyano-2-fluorophenyl)-4-phenyl-1H-pyrrole-3-sulfonamide (215 mg, 0.512 mmol) in 1,4-dioxane/water (4:1, 2.5 mL) in a sealed tube was added potassium phosphate (594.66 mg, 1.279 mmol). Reaction mixture was degassed with argon for 10 minutes. Trimethylboroxine (76.73 mg, 0.614 mmol) was added to the reaction mixture followed by Pd(PPh₃)₄ (59.08 mg, 0.051 mmol). The RM was heated at 100° C. for 16 hours. Upon completion, RM was concentrated under reduced pressure and purified by preparative TLC (eluting with 30% ethyl acetate in hexane) to afford 60 mg (33%) of N-(4-cyano-2-fluorophenyl)-5-methyl-4-phenyl-1H-pyrrole-3-sulfonamide (Cpd 461).

TABLE 2
Analytical data of Examples

| | LCMS | | | | ¹H NMR | | |
|---|---|---|---|---|---|---|---|
| Cpd | | Rt | [M − H] | [M + H] | Frequency | | |
| number | Method | [min] | m/z | m/z | [MHz] | Solvent | δ [ppm] |
| Cpd | LC-2 | 3.01 | 354 | | 400 | DMSO- | 11.86 (s, 1H), 10.52 (s, 1H), 7.81 (dd, 1H), 7.64-7.53 |
| 001 | | | | | | d6 | (m, 4H), 7.38 (t, 2H), 7.27-7.18 (m, 1H), 6.73 (d, 1H), |
| | | | | | | | 2.38 (s, 3H). |
| Cpd | LC-2 | 2.51 | 340 | | 400 | DMSO- | 12.18 (s, 1H), 10.53 (s, 1H), 7.81 (d, 1H), 7.72-7.57 (m, |
| 002 | | | | | | d6 | 4H), 7.51 (s, 1H), 7.39 (t, 2H), 7.26 (t, 1H), 6.79 (s, 1H). |
| Cpd | LC-2 | 3.37 | 411 | | 400 | DMSO- | 12.14 (br. s., 1 H), 10.21 (s, 1 H), 7.70 (dd, 1 H), 7.61- |
| 003 | | | | | | d6 | 7.67 (m, 2 H), 7.45 (dd, 1 H), 7.36-7.42 (m, 2 H), 7.31- |
| | | | | | | | 7.36 (m, 1 H), 7.22-7.29 (m, 1 H), 6.75 (t, 1 H) |
| Cpd | LC-2 | 2.56 | 358 | | 400 | DMSO- | 12.26 (s, 1H), 10.57 (s, 1H), 7.84-7.78 (m, 1H), 7.64- |
| 004 | | | | | | d6 | 7.59 (m, 2H), 7.56-7.49 (m, 3H), 7.43 (td, 1H), 7.11- |
| | | | | | | | 7.05 (m, 1H), 6.93-6.89 (m, 1H). |
| Cpd | LC-2 | 2.86 | 354 | | 400 | DMSO- | 12.13 (s, 1H), 10.53 (s, 1H), 7.84-7.76 (m, 1H), 7.64- |
| 005 | | | | | | d6 | 7.56 (m, 2H), 7.50-7.39 (m, 3H), 7.27 (t, 1H), 7.08 (d, |
| | | | | | | | 1H), 6.78-6.74 (m, 1H), 2.32 (s, 3H) |
| Cpd | LC-2 | 2.9 | 374 | | 300 | DMSO- | 12.31 (s, 1H), 10.58 (s, 1H), 7.87-7.74 (m, 2H), 7.62- |
| 006 | | | | | | d6 | 7.57 (m, 4H), 7.41 (t, 1H), 7.31 (d, 1H), 6.93 (s, 1H), |
| Cpd | LC-2 | 2.57 | 358 | | 300 | DMSO- | 12.16 (s, 1H), 10.55 (s, 1H), 7.81 (dd, 1H), 7.78-7.51 |
| 007 | | | | | | d6 | (m, 4H), 7.49 (s, 1H), 7.22 (t, 2H), 6.77(s, 1H). |
| Cpd | LC-2 | 2.87 | 354 | | 400 | DMSO- | 12.11 (s, 1H), 10.54 (s, 1H), 7.82 (dd, 1H), 7.78-7.51 |
| 008 | | | | | | d6 | (m, 4H), 7.49 (s, 1H), 7.20 (t, 2H), 6.73 (t, 1H), 2.30 (s, 3H). |
| Cpd | LC-1 | 3.37 | 340 | | 300 | DMSO- | 11.78 (s, 1H), 10.46 (s, 1H), 7.68 (dd, 1H), 7.54-7.42 |
| 009 | | | | | | d6 | (m, 4H), 7.42-7.34 (m, 1H), 7.34-7.17 (m, 3H), 7.08- |
| | | | | | | | 7.00 (m , 1H). |
| Cpd | LC-1 | 3.41 | 370 | | 300 | DMSO- | 12.18 (s, 1H), 10.55 (s, 1H), 7.81 (d, 1H), 7.62-7.52 (m, |
| 010 | | | | | | d6 | 3H), 7.32-7.20 (m, 3H), 6.83 (d, 2H), 3.79 (s, 3H). |
| Cpd | LC-2 | 2.92 | 374 | | 300 | DMSO- | 12.27 (s, 1H), 10.57 (s, 1H), 7.88-7.77 (m, 1H), 7.71- |
| 011 | | | | | | d6 | 7.51 (m, 5H), 7.51-7.40 (m, 2H), 6.84 (d, 1H). |
| Cpd | LC-2 | 2.51 | 370 | | 400 | DMSO- | 12.05 (s, 1H), 10.52 (s, 1H), 7.86-7.78 (m, 1H), 7.66- |
| 012 | | | | | | d6 | 7.54 (m, 4H), 7.46 (dd, 1H), 7.00-6.93 (m, 2H), 6.65 |
| | | | | | | | (s, 1H), 3.77 (s, 3H). |
| Cpd | LC-2 | 1.94 | 368 | 370 | 400 | DMSO- | 12.96 (s, 1H), 10.57 (s, 1H), 7.86 (dd, 1H), 7.81-7.83 |
| 013 | | | | | | d6 | (m, 3H), 7.72-7.66 (m, 1H), 7.64 (dd, 1H), 7.62-7.54 |
| | | | | | | | (m, 3H), 6.96 (s, 1H). |
| Cpd | LC-2 | 2.78 | 376 | | 400 | DMSO- | 12.17 (s, 1H), 10.55 (s, 1H), 7.82 (d, 1 H, J = 10.8 Hz), |
| 014 | | | | | | d6 | 7.74-7.70 (m, 1 H), 7.64-7.57 (m, 3 H), 7.33-7.23 (m, 3 |
| | | | | | | | H), 6.78-6.78 (m, 1 H) |
| Cpd | LC-2 | 2.83 | 354 | | 300 | DMSO- | 11.94 (s, 1H), 10.49 (s, 1H), 7.83 (d, 1H), 7.68-7.60 (m, |
| 015 | | | | | | d6 | 2H), 7.49 (s, 1H), 7.35-7.22 (m, 4H), 6.47 (s, 1H), 2.30 |
| | | | | | | | (s, 3H). |
| Cpd | LC-2 | 2.75 | 374 | | 400 | DMSO- | 2.13 (s, 1H), 10.54 (s, 1H), 7.87-7.79 (m, 1H), 7.66- |
| 016 | | | | | | d6 | 7.52 (m, 5H), 7.43-7.33 (m, 2H), 6.79 (dd, 1H). |
| Cpd | LC-2 | 2.76 | 370 | | 400 | DMSO | 11.80 (s, 1H), 10.52 (s, 1H), 7.82 (d, 1H), 7.69-7.58 (m, |
| 017 | | | | | | d6 | 3H), 7.43 (s, 1H), 7.27 (t, 1H), 7.10 (d, 1H), 7.00 (t, 1H), |
| | | | | | | | 6.83 (s, 1H), 3.86 (s, 3H). |
| Cpd | LC-2 | 2.84 | 332 | | 400 | DMSO- | 11.47 (s, 1H), 10.38 (s, 1H), 7.86-7.74 (m, 1H), 7.64- |
| 018 | | | | | | d6 | 7.52 (m, 2H), 7.29 (dd, 1H), 6.04 (s, 1H), 2.91 (p, 1H), |
| | | | | | | | 1.98-1.85 (m, 2H), 1.70-1.40 (m, 6H). |
| Cpd | LC-2 | 2.77 | 354 | | 400 | DMSO- | 11.57 (s, 1H), 10.37 (s, 1H), 7.79 (d, 1H), 7.56 (d, 2H), |
| 019 | | | | | | d6 | 7.32-7.14 (m, 6H), 6.01 (s, 1H), 3.83 (s, 2H). |
| Cpd | LC-2 | 2.28 | 358 | | 400 | DMSO- | 12.87 (s, 1H), 10.78 (s, 1H), 7.85 (dd, 1H), 7.66 (dd, |
| 020 | | | | | | d6 | 1H), 7.62-7.52 (m, 3H), 7.38 (t, 2H), 7.30-7.21 (m, 1H), |
| | | | | | | | 6.62 (d, 1H). |
| Cpd | LC-2 | 3.1' | 408 | | 300 | DMSO- | 12.43 (s, 1H), 10.58 (s, 1H), 8.05 (s, 1H), 7.99-7.96 (m, |
| 021 | | | | | | d6 | 1H), 7.88-7.78 (m, 1H), 7.66-7.59 (m, 5H), 7.02 (t, 1H). |
| Cpd | LC-2 | 3.5 | 382 | | 400 | DMSO- | 12.17 (s, 1H), 10.52 (s, 1H), 7.86-7.78 (m, 1H), 7.67- |
| 022 | | | | | | d6 | 7.57 (m, 2H), 7.56-7.49 (m, 2H), 7.44 (d, 1H), 7.30 (t, |
| | | | | | | | 1H), 7.14 (d, 1H), 6.79 (dd, 1H), 2.90 (hept, 1H), 1.23 |
| | | | | | | | (d, 6H). |
| Cpd | LC-2 | 2.39 | 358 | | 400 | DMSO- | 12.87 (s, 1H), 10.78 (s, 1H), 7.85 (d, 1H), 7.65-7.57 (m, |
| 023 | | | | | | d6 | 4H), 7.44 (t, 2H), 7.38 (t, 1H), 7.29 (t, 1H). |
| Cpd | LC-2 | 1.17 | 341 | 343 | 400 | DMSO- | 12.50 (s, 1H), 10.62 (s, 1H), 8.57-8.51 (m, 2H), 7.81 |
| 024 | | | | | | d6 | (d, 1H), 7.68-7.58 (m, 5H), 7.11 (d, 1H). |
| Cpd | LC-2 | 2.22 | 365 | | 400 | DMSO- | 12.37 (s, 1H), 10.61 (s, 1H), 8.17 (s, 1H), 8.00 (d, 1H), |
| 025 | | | | | | d6 | 7.84-7.81 (m, 1H), 7.71 (d, 1H), 7.67-7.54 (m, 4H), |
| | | | | | | | 7.02 (t, 1H). |
| Cpd | LC-2 | 1.77 | 341 | 343 | 300 | CDCl3 | 12.40 (s, 1H), 10.58 (s, 1H), 8.54 (s, 1H), 7.80 (s, 3H), |
| 026 | | | | | | | 7.63 (s, 2H), 7.45 (s, 1H), 7.25 (s, 1H), 7.07 (s, 1H). |
| Cpd | LC-2 | 1.33 | 341 | 343 | 400 | DMSO- | 12.25 (s, 1H), 10.59 (s, 1H), 8.89 (d, 1H), 8.44 (dd, 1H), |
| 027 | | | | | | d6 | 8.03 (dt, 1H), 7.72 (d, 1H), 7.62-7.50 (m, 3H), 7.41 |
| | | | | | | | (dd, 1H), 6.92 (s, 1H). |
| Cpd | LC-2 | 2.15 | 358 | | 300 | DMSO- | 12.24 (s, 1H), 10.90 (brs, 1H), 7.92 (t, 1H), 7.70-7.65 |
| 028 | | | | | | d6 | (m, 3H), 7.52-7.30 (m, 3H), 7.21(d, 1H), 6.82 (s, 1H). |
| Cpd | LC-2 | 2.85 | 336 | | 300 | DMSO- | 12.12 (s, 1H), 9.63 (s, 1H), 7.70-7.60 (m, 4H), 7.45- |
| 029 | | | | | | d6 | 7.35 (m, 4H), 7.28-7.19 (m, 1H), 6.77 (s, 1H), 2.19 (s, 3H). |
| Cpd | LC-2 | 2.88 | 352 | | 300 | DMSO- | 12.13 (s, 1H), 9.50 (s, 1H), 7.63 (d, 2H), 7.57-7.47 |
| 030 | | | | | | d6 | (m, 2H), 7.46-7.32 (m, 4H), 7.25 (t, 1H), 6.81 (s, 1H), |
| | | | | | | | 3.80 (s, 3H). |
| Cpd | LC-2 | 2.82 | 336 | | 300 | DMSO- | 12.15 (s, 1H), 10.63 (s, 1H), 7.68-7.54 (m, 4H), 7.38 |
| 031 | | | | | | d6 | (dd, 2H), 7.29-7.21 (m, 1H), 7.18-7.09 (m, 2H), 6.76 |
| | | | | | | | (s, 1H), 2.39 (s, 3H). |
| Cpd | LC-2 | 2.82 | 345 | | 400 | DMSO- | 12.01 (s, 1H), 9.34 (s, 1H), 7.67-7.61 (m, 2H), 7.40 (t, |
| 032 | | | | | | d6 | 2H), 7.25 (t, 1H), 7.18 (dd, 1H), 7.12 (t, 1H), 6.80 (dd, |
| | | | | | | | 1H), 6.74-6.65 (m, 2H), 3.71 (s, 3H). |
| Cpd | LC-2 | 2.82 | 354 | | 400 | DMSO- | 11.85 (s, 1H), 10.65 (s, 1H), 7.81 (d, 1H), 7.64-7.57 (m, |
| 033 | | | | | | d6 | 2H), 7.51-7.39 (m, 5H), 7.32-7.30 (m, 1H), 2.24 (s, 3H). |
| Cpd | LC-2 | 2.83 | 354 | | 400 | DMSO- | 11.47 (s, 1H), 10.55 (s, 1H), 7.78 (d, 1H), 7.60-7.50 |
| 034 | | | | | | d6 | (m, 2H), 7.39 (dd, 1H), 7.26-7.20 (m, 2H), 7.16 (dt, |
| | | | | | | | 3H), 6.37 (s, 1H), 3.89 (s, 2H). |
| Cpd | LC-2 | 2.87 | 340 | | 300 | DMSO- | 12.24 (s, 1H), 11.04 (s, 1H), 7.77 (dd, 1H), 7.72-7.61 |
| 035 | | | | | | d6 | (m, 3H), 7.48-7.35 (m, 2H), 7.35-7.21 (m, 1H), 7.14 |
| | | | | | | | (td, 2H), 6.80 (dd, 1H). |
| Cpd | LC-2 | 2.73 | 356 | | 300 | DMSO- | 12.20 (s, 1H), 10.04 (s, 1H), 8.02 (d, 1H), 7.76 (dd, 1H), |
| 036 | | | | | | d6 | 7.70-7.59 (m, 3H), 7.52 (d, 1H), 7.44-7.34 (m, 2H), |
| | | | | | | | 7.32-7.21 (m, 1H), 6.84 (s, 1H). |
| Cpd | LC-2 | 1.73 | 323 | | 300 | DMSO- | 12.12 (s, 1H), 8.65 (d, 1H), 8.03 (dd, 1H), 7.76-7.60 |
| 037 | | | | | | d6 | (m, 2H), 7.56 (s, 1H), 7.43-7.33 (m, 2H), 7.30-7.19 |
| | | | | | | | (m, 1H), 7.16 (d, 1H), 6.84 (s, 1H). |
| Cpd | LC-2 | 2.76 | 376 | | 300 | DMSO- | 12.20 (s, 1H), 10.57 (s, 1H), 7.93-7.69 (m, 2H), 7.69- |
| 038 | | | | | | d6 | 7.55 (m, 3H), 7.38 (ddd, 1H), 7.19 (td, 1H), 6.75 (d, 1H). |
| Cpd | LC-2 | 3.06 | 392 | | 400 | DMSO- | 12.30 (s, 1H), 10.60 (s, 1H), 7.83 (d, 1H), 7.73-7.67 |
| 039 | | | | | | d6 | (m, 1H), 7.66-7.60 (m, 3H), 7.57-7.46 (t, 1H), 7.29 |
| | | | | | | | (t, 1H), 6.84 (s, 1H). |
| Cpc | LC-2 | 2.82 | 376 | | 400 | DMSO- | 12.30 (s, 1H), 10.60 (s, 1H), 7.82 (dd, 1H), 7.67-. 58 (m, |
| 040 | | | | | | d6 | 3H), 7.55 (ddt, 1H), 7.41-7.31 (m, 1H), 7.31-7.22 (m, |
| | | | | | | | 1H), 6.84 (d, 1H). |
| Cpd | LC-2 | 2.78 | 376 | | 300 | DMSO- | 12.19 (s, 1H), 10.61 (s, 1H), 7.85-7.72 (m, 1H), 7.70- |
| 041 | | | | | | d6 | 7.52 (m, 4H), 7.39-7.30 (m, 1H), 7.20-7.10 (m, 1H), |
| | | | | | | | 6.85 (d, 1H). |
| Cpc | LC-2 | 3.1 | 372 | | 300 | DMSO- | 12.15 (s, 1H), 10.57 (s, 1H), 7.83 (dd, 1H), 7.69-7.48 |
| 042 | | | | | | d6 | (m, 4H), 7.24-7.12 (m, 2H), 6.78 (d, 1H), 2.28 (s, 3H). |
| Cpc | LC-2 | 3.12 | 372 | | 300 | DMSO- | 12.12 (s, 1H), 10.56 (s, 1H), 7.87-7.77 (m, 1H), 7.66- |
| 043 | | | | | | d6 | 7.52 (m, 4H), 7.19-7.03 (m, 2H), 6.73 (d, 1H), 2.32 |
| | | | | | | | (s, 3H). |
| Cpd | LC-2 | 3.11 | 372 | | 400 | DMSO- | 12.15 (s, 1H), 10.55 (s, 1H), 7.86-7.79 (m, 1H), 7.66- |
| 044 | | | | | | d6 | 7.58 (m, 2H), 7.58-7.52 (m, 2H), 7.25-7.07 (m, 2H), |
| | | | | | | | 6.77 (g, 1H), 2.31 (s, 3H). |
| Cpd | LC-2 | 3.2 | 392 | | 400 | DMSO- | 12.26 (s, 1H), 10.58 (s, 1H), 7.83 (d, 1H), 7.76 (t, 1H), |
| 045 | | | | | | d6 | 7.67-7.58 (m, 3H), 7.54 (dd, 1H), 7.37 (dd, 1H), 6.81 |
| | | | | | | | (q, 1H). |
| Cpc | LC-2 | 3.1 | 392 | | 300 | DMSO- | 12.31 (s, 1H), 10.59 (s, 1H), 7.92-7.78 (m, 2H), 7.63 |
| 046 | | | | | | d6 | (ddd, 3H), 7.42-7.29 (m, 2H), 6.87 (td, 1H). |
| Cp | LC-2 | 3.4 | 369 | | 400 | DMSO- | 12.12 (s, 1H), 10.01 (s, 1H), 7.74 (td, 1H), 7.57 (td, 1H), |
| 047 | | | | | | d6 | 7.43 (dd, 1H), 7.41-7.22 (m, 4H), 6.73 (td, 1H). |
| Cp | LC-2 | 2.47 | 368 | 370 | 300 | DMSO- | 12.06 (s, 1H), 10.89 (s, 1H), 8.18 (d, 1H), 8.04-7.91 |
| 048 | | | | | | d6 | (m, 1H), 7.74 (t, 1H), 7.54 (dd, 1H), 7.40-7.18 (m, |
| | | | | | | | 3H), 6.88 (q, 1H). |
| Cp | LC-2 | 2.92 | 359 | | 300 | DMSO- | 12.03 (s, 1H), 9.62 (s, 1H), 7.73 (td, 1H), 7.38-7.22 |
| 049 | | | | | | d6 | (m, 4H), 6.73 (q, 4H), 5.92 (s, 2H). |
| Cpc | LC-2 | 3.16 | 373 | | 300 | DMSO- | 12.09 (s, 1H), 10.56 (s, 1H), 7.92-7.45 (m, 5H), 7.37- |
| 050 | | | | | | d6 | 7.15 (m, 3H), 6.84 (s, 1H). |
| Cpd | LC-2 | 3.26 | 351 | | 400 | DMSO- | 12.12 (s, 1H), 10.11 (s, 1H), 7.73 (td, 1H), 7.49 (dd, |
| 051 | | | | | | d6 | 1H), 7.36-7.14 (m, 5H), 6.97 (ddt, 1H), 6.75 (td, 1H). |
| Cpc | LC-2 | 3.62 | 367 | | 300 | DMSO- | 12.10 (s, 1H), 9.91 (s, 1H), 7.80-7.69 (m, 1H), 7.44- |
| 052 | | | | | | d6 | 7.21 (m, 7H), 6.72 (dd, 1H ). |
| Cpd | LC-2 | 2.77 | 372 | | 400 | DMSO- | 12.06 (s, 1H), 9.85 (s, 1H), 7.73 (td, 1H), 7.42-7.33 |
| 053 | | | | | | d6 | (m, 2H), 7.37-7.22 (m, 3H), 7.18 (dd, 1H), 7.12 (dd, |
| | | | | | | | 1H), 6.74 (d, 1H), 3.99 (s, 2H). |
| Cpc | LC-2 | 3.23 | 351 | | 300 | DMSO- | 12.07 (s, 1H), 9.71 (s, 1H), 7.75 (t, 1H), 7.40-7.20 (m, |
| 054 | | | | | | d6 | 6H), 7.03 (t, 1H), 6.64 (s, 1H). |
| Cpd | LC-2 | 3.85 | 401 | | 300 | DMSO- | 12.17 (s, 1H), 10.38 (s, 1H), 7.79-7.60 (m, 3H), 7.58- |
| 055 | | | | | | d6 | 7.48 (m, 2H), 7.40-7.20 (m, 3H), 6.79 (s, 1H). |
| Cpd | LC-2 | 3.25 | 351 | | 300 | DMSO- | 12.12 (s, 1H), 10.08 (s, 1H), 7.79-7.68 (m, 1H), 7.42 |
| 056 | | | | | | d6 | (dd, 1H), 7.39-7.22 (m, 3H), 7.22-7.06 (m, 3H), 6.74 |
| | | | | | | | (s, 1H). |
| Cpd | LC-2 | 3.42 | 347 | | 300 | DMSO- | 12.02 (s, 1H), 9.59 (s, 1H), 7.73 (t, 1H), 7.31-7.29 (m, |
| 057 | | | | | | d6 | 4H), 7.18 (t, 1H), 7.04-6.86 (m, 2H), 6.72 (q, 1H), 2.24 |
| | | | | | | | (s, 3H). |
| Cpd | LC-2 | 3.12 | 374 | | 400 | DMSO- | 12.25 (s, 1H), 11.01 (s, 1H), 7.84 (d, 1H), 7.75-7.69 (m, |
| 058 | | | | | | d6 | 2H), 7.55-7.26 (m, 5H), 6.77 (s, 1H). |
| Cpd | LC-2 | 2.93 | 370 | | 400 | DMSO- | 12.19 (s, 1H), 10.70 (s, 1H), 7.73 (t, 1H), 7.64 (s, 1H), |
| 059 | | | | | | d6 | 7.57 (d, 1H), 7.34-7.24 (m, 3H), 6.94 (s, 1H), 6.85 (d, |
| | | | | | | | 1H), 6.77 (s, 1H), 3.84 (s, 3H). |
| Cpd | LC-2 | 3.28 | 414 | 416 | 400 | DMSO- | 12.07 (s, 1H), 10.40 (s, 1H), 8.17 (s, 1H), 7.74 (td, 1H), |
| 060 | | | | | | d6 | 7.62 (dd, 1H), 7.53 (s, 1H), 7.35-7.21 (m, 3H), 6.94 |
| | | | | | | | (dt, 1H), 3.88 (s, 3H). |
| Cpd | LC-2 | 2.16 | 367 | 369 | 400 | DMSO- | 12.03 (s, 1H), 9.73 (s, 1H), 7.81 (d, 1H), 7.65 (t, 2H), |
| 061 | | | | | | d6 | 7.51 (dd, 1H), 7.39 (t, 2H), 7.31 (d, 1H), 7.24 (t, 1H), |
| | | | | | | | 6.90 (s, 1H), 3.95 (s, 2H), 3.81 (s, 3H). |
| Cpd | LC-2 | 3.04 | 406 | 408 | 400 | DMSO- | 12.05 (s, 1H), 9.86 (s, 1H), 7.95 (d, 1H), 7.68-7.61 |
| 062 | | | | | | d6 | (m, 2H), 7.58-7.49 (m, 2H), 7.39 (t, 2H), 7.24 (t, 1H), |
| | | | | | | | 6.89 (dd, 1H), 3.82 (s, 3H). |
| Cpd | LC-2 | 3.26 | 370 | | 300 | DMSO- | 12.20 (s, 1H), 9.92 (s, 1H), 7.72-7.60 (m, 3H), 7.50- |
| 063 | | | | | | d6 | 7.33 (m, 4H), 7.30-7.20 (m, 1H), 6.84 (s, 1H), 3.79 (s, 3H). |
| Cpd | LC-2 | 2.46 | 358 | | 400 | DMSO- | 12.09 (s, 1H), 9.98 (s, 1H), 7.81-7.65 (m, 4H), 7.40- |
| 064 | | | | | | d6 | 7.25 (m, 4H), 6.73 (s, 1H). |
| Cpd | LC-2 | 2.98 | 388 | | 300 | DMSO- | 12.09 (s, 1H), 9.81 (s, 1H), 7.65 (d, 2H), 7.44-7.14 |
| 065 | | | | | | d6 | (m, 6H), 6.70 (d, 1H), 5.22 (s, 2H). |
| Cpd | LC-2 | 1.52 | 359 | 361 | 400 | DMSO- | 12.51 (br. s., 1 H), 10.63 (br. s., 1 H), 8.61 (d, 1 H), |
| 066 | | | | | | d6 | 8.43 (d, 1 H), 7.75-7.92 (m, 2 H), 7.71 (s, 1 H), 7.48- |
| | | | | | | | 7.66 (m, 2 H), 7.03 (s, 1 H) |
| Cpd | LC-2 | 3.24 | 368 | | 300 | DMSO- | 11.43 (s, 1H), 10.47 (s, 1H), 7.71 (d, 1H), 7.50 (d, 2H), |
| 067 | | | | | | d6 | 7.34 (d, 1H), 7.18-6.97 (m, 5H), 3.90 (s, 2H), 1.91 (s, 3H). |
| Cpd | LC-2 | 2.37 | 350 | 352 | 300 | DMSO- | 11.99 (br. s., 1 H), 10.87 (br. s., 1 H), 8.16 (s, 1 H), |
| 068 | | | | | | d6 | 7.88 (d, 1 H), 7.57-7.74 (m, 2 H), 7.30-7.52 (m, 3 H), |
| | | | | | | | 7.11-7.30 (m, 1 H), 6.76-6.94 (m, 1 H) |
| Cpd | LC-2 | 2.79 | 341 | 343 | 300 | DMSO- | 12.03 (br. s., 1 H), 9.57 (br. s., 1 H), 7.53-7.72 (m, 2 |
| 069 | | | | | | d6 | H), 7.40 (t, 2 H), 7.14-7.32 (m, 2 H), 6.57-6.82 (m, 4 |
| | | | | | | | H), 5.92 (s, 2 H) |
| Cpd | LC-2 | 3.46 | 347 | | 300 | DMSO- | 12.09 (br. s., 1 H), 9.87 (s, 1 H), 7.64 (d, 2 H), 7.31- |
| 070 | | | | | | d6 | 7.52 (m, 3 H), 7.20-7.31 (m, 1 H), 7.09 (dd, 1 H), 7.14 |
| | | | | | | | (dd, 1 H), 6.72 (s, 1 H), 2.08-2.25 (m, 3 H) |
| Cpd | LC-2 | 3.56 | 401 | | 400 | DMSO- | 12.2 (bs, 1H), 10.72 (s, 1H), 7.72-7.69 (m, 1H), 7.66- |
| 071 | | | | | | d6 | 7.6 (m, 3H), 7.51-7.46 (m, 1H), 7.4-7.36 (m, 2H), 7.27- |
| | | | | | | | 7.23 (m, 1H), 6.82-6.8 (t, 1H, J = 2.04 Hz) |
| Cpd | LC-2 | 2.89 | 358 | | 300 | DMSO- | 12.23 (br. s., 1 H), 11.05 (br. s., 1 H), 7.61-7.87 (m, 3 |
| 072 | | | | | | d6 | H), 7.21-7.46 (m, 3 H), 6.99-7.21 (m, 2 H), 6.77 (s, 1 H) |
| Cpd | LC-2 | 3.55 | 419 | | 300 | DMSO- | 12.22 (br. s., 1 H), 10.76 (br. s., 1 H), 7.60-7.84 (m, 3 |
| 073 | | | | | | d6 | H), 7.40-7.60 (m, 1 H), 7.11-7.40 (m, 3 H), 6.82 (d, 1 H) |
| Cpd | LC-2 | 2.28 | 376 | | 300 | DMSO- | 12.25 (br. s., 1 H), 10.95 (br. s., 1 H), 7.94 (dd, 1 H), |
| 074 | | | | | | d6 | 7.64-7.83 (m, 2 H), 7.50 (dd, 1 H), 7.09-7.43 (m, 3 |
| | | | | | | | H), 6.81 (s, 1 H) |
| Cpd | LC-2 | 3.53 | 365 | 367 | 300 | DMSO- | 12.08 (s, 1H), 9.91 (s, 1H), 7.73 (t, 1H), 7.41 (s, 1H), |
| 075 | | | | | | d6 | 7.37-7.21 (m, 3H), 7.17-7.06 (m, 2H), 6.74 (d, 1H), 2.15 |
| | | | | | | | (s, 3H). |
| Cpd | LC-2 | 1.68 | 376 | | 300 | DMSO- | 12.23 (s, 1H), 11.28 (s, 1H), 7.91 (dd, 1H), 7.64-7.56 |
| 076 | | | | | | d6 | (m, 2H), 7.52 (t, 1H), 7.52-7.38 (m, 3H), 7.35-7.23 |
| | | | | | | | (m, 1H). |
| Cpd | LC-2 | 1.62 | 376 | | 300 | DMSO- | 12.96 (s, 1H), 11.28 (s, 1H), 7.95 (dd, 1H), 7.67-7.56 |
| 077 | | | | | | d6 | (m, 2H), 7.51-7.34 (m, 3H), 7.31-7.21 (m, 1H), 6.68 |
| | | | | | | | (d, 1H). |
| Cpd | LC-2 | 0.72 | 344 | 346 | 300 | DMSO | 12.17 (s, 1H), 10.51 (s, 1H), 7.82 (dd, 2H), 7.55-7.65 |
| 078 | | | | | | d6 | (m, 3H), 7.41 (s, 1H), 6.67(s, 1H), 6.49 (s, 1H), 3.86 (s, 3H). |
| Cpd | LC-2 | 2.61 | 408 | | 300 | DMSO- | 12.17 (s, 1H), 10.51 (s, 1H), 7.82 (dd, 2H), 7.55-7.65 |
| 079 | | | | | | d6 | (m, 3H), 7.41 (s, 1H), 6.67(s, 1H), 6.49 (s, 1H), 3.86 (s, 3H). |
| Cpd | LC-2 | 2.3 | 384 | | 300 | DMSO- | 11.92 (s, 1H), 10.53 (s, 1H), 7.88-7.78 (m, 1H), 7.64 |
| 080 | | | | | | d6 | (dd, 2H), 7.55-7.29 (m, 5H), 6.56 (dd, 1H), 4.31 (s, |
| | | | | | | | 2H), 3.25 (s, 3H). |
| Cpd | LC-2 | 0.87 | 344 | | 400 | DMSO- | 12.07 (s, 1H), 10.48 (s, 1H), 7.84-7.76 (m, 1H), 7.68 (d, |
| 081 | | | | | | d6 | 1H), 7.65-7.55 (m, 2H), 7.33 (dd, 1H), 6.58 (dd, 1H), |
| | | | | | | | 6.51 (d, 1H), 3.84 (s, 3H). |
| Cpd | LC-2 | 2.41 | 372 | | 300 | DMSO- | 11.87 (s, 1H), 10.42 (s, 1H), 7.81 (dd, 1H), 7.68-7.55 |
| 082 | | | | | | d6 | (m, 2H), 7.49 (dd, 1H), 7.32 (td, 1H), 7.12 (dd, 2H), |
| | | | | | | | 6.40 (t, 1H), 2.16 (s, 3H). |
| Cpd | LC-2 | 2.41 | 388 | 390 | 300 | DMSO- | 11.69 (s, 1H), 10.49 (s, 1H), 7.86-7.76 (m, 1H), 7.63 |
| 083 | | | | | | d6 | (dd, 2H), 7.44 (dd, 1H), 7.33 (td, 1H), 6.97-6.91 (m, |
| | | | | | | | 2H), 6.68 (g, 1H), 3.85 (s, 3H). |
| Cpd | LC-2 | 2.25 | 388 | | 400 | DMSO- | 12.21 (s, 1H), 10.56 (s, 1H), 7.86-7.79 (m, 1H), 7.70- |
| 084 | | | | | | d6 | 7.60 (m, 3H), 7.30 (dd, 1H), 7.21 (dd, 1H), 6.90-6.80 |
| | | | | | | | (m, 2H), 3.79 (s, 3H). |
| Cpd | LC-2 | 2.78 | 368 | | 300 | DMSO- | 11.65 (s, 1H), 10.33 (s, 1H), 7.85-7.75 (m, 1H), 7.66- |
| 085 | | | | | | d6 | 7.53 (m, 2H), 7.40 (dd, 1H), 7.19 (dd, 1H), 7.10 (d, 2H), |
| | | | | | | | 6.15 (dd, 1H), 1.97 (s, 6H). |
| Cpd | LC-2 | 2.27 | 388 | 390 | 400 | DMSO- | 12.01 (s, 1H), 10.50 (s, 1H), 7.84-7.76 (m, 1H), 7.66- |
| 086 | | | | | | d6 | 7.56 (m, 3H), 7.49 (dd, 1H), 6.93 (dd, 1H), 6.86 (dd, |
| | | | | | | | 1H), 6.65 (g, 1H), 3.79 (s, 3H). |
| Cpd | LC-2 | 2.07 | 388 | | 300 | DMSO- | 12.14 (s, 1H), 10.53 (s, 1H), 7.87-7.76 (m, 1H), 7.62 |
| 087 | | | | | | d6 | (dd, 2H), 7.55 (dd, 1H), 7.30-7.03 (m, 3H), 6.78 (d, |
| | | | | | | | 1H), 3.86 (s, 3H). |
| Cpd | LC-2 | 2.37 | 394 | | 300 | DMSO- | 12.19 (s, 1H), 10.54 (s, 1H), 7.91-7.79 (m, 2H), 7.71- |
| 088 | | | | | | d6 | 7.57 (m, 4H), 6.82 (d, 1H). |
| Cpd | LC-2 | 2.5 | 389 | | 400 | DMSO- | 11.79 (s, 1H), 10.55 (s, 1H), 8.13 (dd, 1H), 7.83 (d, 1H), |
| 089 | | | | | | d6 | 7.62 (d, 2H), 7.49 (s, 1H), 7.09 (dd, 1H), 6.79 (s, 1H), |
| | | | | | | | 3.96 (s, 3H). |
| Cpd | LC-2 | 2.14 | 359 | | 300 | DMSO- | 12.47 (s, 1H), 10.58 (s, 1H), 8.45 (d, 1H), 7.90-7.78 (m, |
| 090 | | | | | | d6 | 2H), 7.62 (s, 2H), 7.47 (s, 1H), 7.45-7.32 (m, 1H), 6.97 |
| | | | | | | | (s, 1H). |
| Cpd | LC-2 | 2.69 | 376 | | 300 | DMSO- | 11.91 (s, 1H), 10.57 (s, 1H), 7.76 (d, 1H), 7.61-7.36 (m, |
| 091 | | | | | | d6 | 4H), 7.22 (t, 2H), 6.70 (s, 1H). |
| Cpd | LC-2 | 2.93 | 390 | | 300 | DMSO- | 12.17 (s, 1H), 10.50 (s, 1H), 7.83-7.72 (m, 2H), 7.61- |
| 093 | | | | | | d6 | 7.50 (m, 6H), 7.01-6.65 (m, 1H), 6.48 (t, 1H). |
| Cpd | LC-2 | 1.26 | 342 | | 300 | DMSO- | 12.62 (s, 1H), 10.62(s, 1H), 8.79 (d, 2H), 7.87-7.79 (m, |
| 094 | | | | | | d6 | 1H), 7.61 (t, 2H), 7.45 (t, 1H), 7.33(t, 1H), 7.10(t, 1H). |
| Cpd | LC-2 | 3.02 | 400 | | 300 | DMSO- | 11.49 (s, 1H), 10.46 (s, 1H), 7.81 (d, 1H), 7.70-7.59 (m, |
| 095 | | | | | | d6 | 2H), 7.34 (dd, 1H), 7.26 (t, 1H), 6.74 (d, 2H), 6.71-6.65 |
| | | | | | | | (m, 1H), 3.78 (s, 6H). |
| Cpd | LC-2 | 2.39 | 383 | | 300 | DMSO- | 12.43 (s, 1H), 10.61 (s, 1H), 8.00-7.89 (m, 2H), 7.87- |
| 096 | | | | | | d6 | 7.73 (m, 2H), 7.69 (dd, 1H), 7.66-7.55 (m, 2H), 6.98 |
| | | | | | | | (d, 1H). |
| Cpd | LC-2 | 2.91 | 394 | | 400 | DMSO- | 12.34 (s, 1H), 10.60 (s, 1H), 7.85-7.79 (m, 1H), 7.68- |
| 097 | | | | | | d6 | 7.62 (m, 3H), 7.54-7.41 (m, 2H), 6.92 (q, 1H). |
| Cpd | LC-2 | 2.66 | 318 | | 400 | DMSO- | 11.52 (s, 1H), 10.39 (s, 1H), 7.83-7.75 (m, 1H), 7.58 |
| 098 | | | | | | d6 | (dd, 2H), 7.29 (dd, 1H), 6.10 (s, 1H), 3.42-3.33 (m, 1H), |
| | | | | | | | 2.25-2.12 (m, 2H), 2.10-1.96 (m, 2H), 1.91-1.85 (m, |
| | | | | | | | 1H), 1.85-1.70 (m, 1H). |
| Cpd | LC-2 | 3.05 | 366 | 368 | 400 | DMSO- | 12.20 (br. s., 1 H), 10.84 (s, 1 H), 8.43-8.56 (m, 1 H), |
| 099 | | | | | | d6 | 7.70-7.87 (m, 2 H), 7.60-7.70 (m, 2 H), 7.58 (dd, 1 |
| | | | | | | | H), 7.32-7.44 (m, 2 H), 7.16-7.32 (m, 1 H), 6.70- |
| | | | | | | | 6.87 (m, 1 H) |
| Cpd | LC-2 | 2.04 | 312 | 314 | 400 | DMSO- | 12.07 (br. s., 1 H), 9.38 (s, 1 H), 8.23 (d, 1 H), 7.64 (d, |
| 100 | | | | | | d6 | 2 H), 7.47 (d, 1 H), 7.39 (t, 2 H), 7.05-7.31 (m, 3 H), |
| | | | | | | | 6.68 (s, 1 H), 2.30 (s, 3 H). |
| Cpd | LC-2 | 2.27 | 326 | 328 | 300 | DMSO- | 12.04 (br. s., 1 H), 9.23 (s, 1 H), 7.64 (d, 2 H), 7.39 (t, 2 |
| 101 | | | | | | d6 | H), 7.19-7.34 (m, 2 H), 7.16 (br. s., 1 H), 7.00 (d, 1 H), |
| | | | | | | | 6.68 (s, 1 H), 2.29-2.40 (m, 3 H), 2.25 (s, 3 H). |
| Cpd | LC-2 | 2.49 | 346 | | 400 | DMSO- | 12.23 (br. s., 1 H), 10.51 (s, 1 H), 7.75-7.89 (m, 1 H), |
| 102 | | | | | | d6 | 7.55-7.69 (m, 2 H), 7.49 (dd, 1 H), 7.45 (dd, 1 H), 7.32 |
| | | | | | | | (dd, 1 H), 7.07 (dd, 1 H), 6.53 (t, 1 H). |
| Cpd | LC-2 | 2.51 | 346 | | 400 | DMSO- | 12.09 (br. s., 1 H), 10.50 (s, 1 H), 7.75-7.86 (m, 1 H), |
| 103 | | | | | | d6 | 7.70 (dd, 1 H), 7.51-7.64 (m, 3 H), 7.47 (dd, 1 H), 7.43 |
| | | | | | | | (dd, 1 H), 6.67 (t, 1 H). |
| Cpd | LC-2 | 2.5 | 376 | | 400 | DMSO- | 12.33 (br. s., 1 H), 10.58 (s, 1 H), 7.74-7.89 (m, 1 H), |
| 104 | | | | | | d6 | 7.53-7.67 (m, 3 H), 7.38-7.53 (m, 2 H), 7.11 (tt, 1 H), |
| | | | | | | | 7.03 (dd, 1 H). |
| Cpd | LC-2 | 2.43 | | 361 | 400 | DMSO- | 12.34 (br. s., 1 H), 10.58 (s, 1 H), 8.26 (ddd, 1 H), 8.13 |
| 105 | | | | | | d6 | (dt, 1 H), 7.76-7.90 (m, 1 H), 7.54-7.71 (m, 3 H), 7.44 |
| | | | | | | | (ddd, 1 H), 6.78-6.95 (m, 1 H). |
| Cpd | LC-2 | 2.86 | 365 | | 400 | DMSO- | 12.40 (br. s., 1 H), 10.63 (s, 1 H), 7.85-7.98 (m, 1 H), |
| 106 | | | | | | d6 | 7.79-7.85 (m, 1 H), 7.69-7.79 (m, 2 H), 7.67 (dd, 1 |
| | | | | | | | H), 7.57-7.65 (m, 2 H), 7.49 (td, 1 H), 7.05 (dd, 1 H). |
| Cpd | LC-2 | 1.65 | 388 | | 400 | DMSO- | 12.15 (br. s., 1 H), 10.55 (s, 1 H), 7.75-7.88 (m, 1 H), |
| 107 | | | | | | d6 | 7.57-7.69 (m, 3 H), 7.55 (dd, 1 H), 7.39 (t, 1 H), 7.13- |
| | | | | | | | 7.30 (m, 1 H), 6.78 (q, 1 H), 5.31 (t, 1 H), 4.59 (d, 2 H). |
| Cpd | LC-2 | 1.78 | 388 | | 400 | DMSO- | 12.19 (br. s., 1 H), 10.54 (s, 1 H), 7.76-7.89 (m, 1 H), |
| 108 | | | | | | d6 | 7.64-7.71 (m, 1 H), 7.56-7.64 (m, 2 H), 7.54 (dd, 1 |
| | | | | | | | H), 7.18-7.33 (m, 2 H), 6.77 (q, 1 H), 5.27 (t, 1 H), |
| | | | | | | | 4.49 (d, 2 H). |
| Cpd | LC-2 | 3.4 | 369 | | 400 | DMSO- | 12.06 (s, 1H), 9.66 (bs, 1H), 7.69-7.66 (m, 2H), 7.57- |
| 109 | | | | | | d6 | 7.49 (m, 1H), 7.37-7.3 (m, 2H), 7.25-7.2 (m, 2H), 6.67 |
| | | | | | | | (s, 1H) |
| Cpd | LC-2 | 3.42 | 369 | | 400 | DMSO- | 12.17 (s, 1H), 9.95 (s, 1H), 7.58-7.31 (m, 5H), 7.09 (t, |
| 110 | | | | | | d6 | 1H, J = 7.4 Hz), 6.81 (s, 1H). |
| Cpd | LC-2 | 3.57 | 365 | | 400 | DMSO- | 11.82 (s, 1H), 9.87 (s, 1H), 7.59-7.52 (m, 1H), 7.34- |
| 111 | | | | | | d6 | 7.29 (m, 3H), 7.28-7.22 (m, 3H), 6.36 (s, 1H), 2.29 (s, 3H) |
| Cpd | LC-2 | 2.38 | 348 | 350 | 400 | DMSO- | 12.04 (br. s., 1 H), 10.35 (s, 1 H), 8.74 (dd, 1 H), 8.21- |
| 112 | | | | | | d6 | 8.35 (m, 1 H), 7.89 (d, 1 H), 7.69 (d, 1 H), 7.51-7.65 |
| | | | | | | | (m, 3 H), 7.49 (dd, 1 H), 7.44 (dd, 1 H), 7.30-7.40 (m, |
| | | | | | | | 2 H), 7.15-7.27 (m, 1 H), 6.77 (t, 1 H). |
| Cpd | LC-2 | 3.61 | 419 | 421 | 400 | DMSO- | 12.12 (s, 1H), 10.71 (s, 1H), 7.71-7.67 (m, 3H), 7.60 |
| 113 | | | | | | d6 | (brs, 1H), 7.49 (dd, 1H), 7.23 (t, 2H), 6.79 (brs, 1H). |
| Cpd | LC-2 | 3.59 | 419 | | 400 | DMSO- | 12.27 (s, 1H), 10.73 (s, 1H) 7.70 (dd, 1H), 7.65 (d, 1H), |
| 114 | | | | | | d6 | 7.55-7.46 (m, 3H), 7.45-7.39 (m, 1H), 7.73 (dt, 1H), |
| | | | | | | | 6.93 (brs, 1H) |
| Cpd | LC-2 | 3.09 | 395 | 397 | 400 | DMSO- | 11.99 (s, 1H), 9.40 (s, 1H), 7.72 (t, 1H), 7.33-7.22 (m, |
| 115 | | | | | | d6 | 4H), 7.12 (t, 1H), 6.85 (dd, 1H), 6.73 (dd, 1H), 6.70 |
| | | | | | | | (brs, 1H), 4.76 (t, 1H), 4.64 (t, 1H), 4.23 (t, 1H), 4.15 (t, |
| | | | | | | | 1H). |
| Cpd | LC-2 | 3.75 | 430 | | 400 | DMSO- | 12.04 (s, 1H), 9.61 (s, 1H), 7.81-7.63 (m, 3H), 7.45- |
| 116 | | | | | | d6 | 7.43 (m, 1H) 7.31-7.25 (m, 3H), 6.74 (s, 1H), 3.71 (s, |
| | | | | | | | 3H). |
| Cpd | LC-2 | 3.66 | 449 | | 400 | DMSO- | 12.22 (s, 1H), 10.72 (s, 1H), 7.75-7.71 (m, 2H), 7.49 |
| 117 | | | | | | d6 | (dd, 1H), 7.29 (dd, 1H), 7.21 (dd, 1H), 6.87-6.83 (m, |
| | | | | | | | 2H), 3.78 (s, 3H) |
| Cpd | LC-2 | 3.86 | 415 | | 400 | DMSO- | 11.93 (s, 1H), 10.63 (s, 1H), 7.72 (dd, 1H), 7.56 (brs, |
| 118 | | | | | | d6 | 1H), 7.49 (dd, 1H), 7.35-7.32 (m, 1H), 7.29-7.23 (m, |
| | | | | | | | 3H), 6.48 (s, 1H), 2.28(s, 3H) |
| Cpd | LC-2 | 3.58 | 437 | | 400 | DMSO- | 12.03 (s, 1H), 10.75 (s, 1H), 7.73 (dd, 1H), 7.67 (brs, |
| 119 | | | | | | d6 | 1H), 7.46 (dd, 1H), 7.52-7.47 (m, 1H), 7.24-7.20 (m, |
| | | | | | | | 2H) 6.73 (brs, 1H). |
| Cpd | LC-2 | 2.98 | 351 | | 300 | DMSO- | 11.91 (s, 1H), 9.67 (s, 1H), 7.40 (m, 1H), 7.33-7.21 (m, |
| 120 | | | | | | d6 | 2H), 7.21-7.07 (m, 3H), 6.98-6.84 (m, 2H), 6.39 (m, 1H). |
| Cpc | LC-2 | 2.97 | 351 | 353 | 300 | DMSO- | 11.82 (s, 1H), 9.59 (s, 1H), 7.53-7.41 (m, 2H), 7.26-7.06 |
| 121 | | | | | | d6 | (m, 4H), 6.98-6.85 (m, 2H), 6.36 (m, 1H). |
| Cpd | LC-2 | 2.5 | 334 | 336 | 400 | DMSO- | 12.24 (s, 1H), 9.69 (s, 1H), 8.54 (m, 1H), 7.85-7.73 (m, |
| 122 | | | | | | d6 | 2H), 7.35-7.15 (m, 4H), 7.08-6.98 (m, 1H), 6.95 (m, 1H). |
| Cpd | LC-2 | 3.2 | 384 | 386 | 400 | DMSO- | 12.36 (s, 1H), 10.38 (s, 1H), 8.54 (m, 1H), 7.86-7.75 |
| 123 | | | | | | d6 | (m, 2H), 7.70-7.62 (m, 2H), 7.53 (dd, J = 8.2, 2.1 Hz, |
| | | | | | | | 1H), 7.39 (dd, J = 3.2, 1.7 Hz, 1H), 7.25 (m, 1H), 7.06 |
| | | | | | | | (m, 1H). |
| Cpd | LC-2 | 2.96 | 402 | 404 | 400 | DMSO- | 12.43 (s, 1H), 10.77 (s, 1H), 8.55 (m, 1H), 7.86-7.77 |
| 124 | | | | | | d6 | (m, 2H), 7.73 (dd, J = 10.3, 6.6 Hz, 1H), 7.58-7.47 (m, |
| | | | | | | | 2H), 7.31-7.21 (m, 1H), 7.11 (m, 1H). |
| Cpd | LC-2 | 2.53 | | 318 | 400 | DMSO- | 12.29 (s, 1H), 10.26 (s, 1H), 8.52 (m, 1H), 7.84-7.73 |
| 125 | | | | | | d6 | (m, 2H), 7.36 (dd, J = 3.1. 1.7 Hz, 1H), 7.33-7.18 (m, |
| | | | | | | | 2H), 7.02-6.92 (m, 3H), 6.81 (m, 1H). |
| Cpd | LC-2 | 2.88 | 346 | 348 | 400 | DMSO- | 12.10 (br. s., 1 H), 7.56-7.73 (m, 2 H), 7.51 (d, 1 H), |
| 126 | | | | | | d6 | 7.35-7.46 (m, 2 H), 7.25 (d, 1 H), 7.29 (d, 2 H), 6.64- |
| | | | | | | | 6.78 (m, 1 H), 2.28 (s, 3 H). |
| Cpd | LC-2 | 2.74 | | 348 | 400 | DMSO- | 12.09 (br. s., 1 H), 9.58 (s, 1 H), 7.59-7.72 (m, 3 H), |
| 127 | | | | | | d6 | 7.34-7.47 (m, 2 H), 7.18-7.34 (m, 3 H), 6.74 (dd, 1 |
| | | | | | | | H), 2.38 (s, 3 H). |
| Cpd | LC-2 | 3.19 | 342 | 344 | 400 | DMSO- | 12.00 (br. s., 1 H), 9.04 (s, 1 H), 7.55-7.69 (m, 2 H), |
| 128 | | | | | | d6 | 7.50 (d, 1 H), 7.33-7.45 (m, 2 H), 7.17-7.33 (m, 2 H), |
| | | | | | | | 6.77 (d, 1 H), 6.71 (s, 1 H), 3.72 (s, 3 H), 2.31 (s, 3 H) |
| Cpd | LC-2 | 2.19 | 326 | 328 | 400 | DMSO- | 12.08 (br. s., 1 H), 9.12 (s, 1 H), 8.16 (d, 1 H), 7.60- |
| 129 | | | | | | d6 | 7.73 (m, 2 H), 7.31-7.45 (m, 2 H), 7.21-7.31 (m, 1 H), |
| | | | | | | | 7.18 (s, 1 H), 7.07 (d, 1 H), 6.66 (s, 1 H), 2.25 (s, 3 H), |
| | | | | | | | 2.09 (s, 3 H) |
| Cpd | LC-2 | 2.99 | 407 | 409 | 400 | DMSO- | 12.00 (s, 1H), 9.38 (s, 1H), 7.72 (t, 1H), 7.33-7.22 (m, |
| 130 | | | | | | d6 | 4H), 7.71 (t, 1H), 6.81 (dd, 1H), 6.71 (dd, 1H), 6.70 |
| | | | | | | | (brs, 1H), 4.04 (dd, 2H), 3.61 (dd, 2H), 3.27 (s, 3H). |
| Cpd | LC-2 | 3.42 | 387 | | 400 | DMSO- | 11.90 (s, 1H), 9.98 (s, 1H), 7.60-7.53 (m, 1H), 7.45- |
| 131 | | | | | | d6 | 7.40 (m, 2H), 7.35-7.29 (m, 1H), 7.25-7.19 (m, 2H), |
| | | | | | | | 6.62 (brs, 1H). |
| Cpd | LC-2 | 4 | 393 | | 400 | DMSO- | 11.48 (s, 1H), 10.53 (s, 1H), 7.70 (dd, 1H), 7.41 (dd, |
| 132 | | | | | | d6 | 1H), 7.38 (brs, 1H), 6.06 (s, 1H), 2.96-2.87 (m, 1H), |
| | | | | | | | 1.93-1.91 (m, 2H), 1.65-1.60 (m, 2H), 1.58-1.55 (m, |
| | | | | | | | 2H), 1.51-1.41 (m, 2H). |
| Cpd | LC-2 | 2.28 | 352 | 354 | 400 | DMSO- | 12.04 (s, 1H), 10.58 (brs, 1H), 8.21 (d, 1H), 7.91 (td, |
| 133 | | | | | | d6 | 1H), 7.73 (t, 1H), 7.49 (d, 1H), 7.32-23 (m, 3H), 6.85 (d, 1H |
| Cpd | LC-2 | 2.93 | 362 | 364 | 400 | DMSO- | 12.04 (br. s., 1 H), 9.84 (s, 1 H), 7.89 (d, 1 H), 7.59- |
| 134 | | | | | | d6 | 7.69 (m, 2 H), 7.50 (dd, 1 H), 7.47 (d, 1 H), 7.34-7.42 |
| | | | | | | | (m, 2 H), 7.20-7.28 (m, 1 H), 6.89 (dd, 1 H), 3.83 (s, 3 H). |
| Cpd | LC-2 | 3.19 | 380 | 382 | 400 | DMSO | 12.16 (br. s., 1 H), 9.85 (s, 1 H), 7.81 (d, 1 H), 7.58- |
| 135 | | | | | | d6 | 7.74 (m, 3 H), 7.33-7.47 (m, 3 H), 7.17-7.33 (m, 1 H), |
| | | | | | | | 6.78 (dd, 1 H), 2.44 (s, 3 H). |
| Cpd | LC-2 | 2.34 | 330 | 332 | 400 | DMSO- | 12.03 (br. s., 1 H), 10.34 (br. s., 1 H), 7.98 (br. s., 1 H), |
| 136 | | | | | | d6 | 7.60-7.69 (m, 2 H), 7.53 (dd, 1 H), 7.45 (dd, 1 H), 7.34- |
| | | | | | | | 7.43 (m, 2 H), 7.15-7.31 (m, 1 H), 6.85 (dd, 1 H), |
| | | | | | | | 2.24 (s, 3 H) |
| Cpd | LC-2 | 3.31 | 351 | | 400 | DMSO- | 12.10 (br. s., 1 H), 9.95 (s, 1 H), 7.62-7.70 (m, 2 H), |
| 137 | | | | | | d6 | 7.56 (td, 1 H), 7.30-7.44 (m, 4 H), 7.19-7.30 (m, 1 H), |
| | | | | | | | 6.72 (dd, 1 H). |
| Cpd | LC-2 | 2.98 | 366 | 368 | 400 | DMSO- | 12.16 (br. s., 1 H), 11.35 (br. s., 1 H), 8.61 (s, 1 H), |
| 138 | | | | | | d6 | 8.05 (dd, 1 H), 7.62-7.71 (m, 2 H), 7.59 (dd, 1 H), 7.33- |
| | | | | | | | 7.43 (m, 2 H), 7.21-7.33 (m, 2 H), 6.87 (dd, 1 H). |
| Cpd | LC-2 | 3.01 | 366 | 368 | 400 | DMSO- | 12.15 (br. s., 1 H), 11.23 (br. s., 1 H), 8.50 (d, 1 H), |
| 139 | | | | | | d6 | 7.60-7.69 (m, 2 H), 7.56 (dd, 1 H), 7.36-7.43 (m, 2 |
| | | | | | | | H), 7.28-7.36 (m, 2 H), 7.21-7.28 (m, 1 H), 6.83 (dd, 1 H). |
| Cpd | LC-2 | 2.19 | 384 | 386 | 400 | DMSO- | 12.16 (br. s., 1 H), 11.37 (br. s., 1 H), 8.55 (br. s., 1 H), |
| 140 | | | | | | d6 | 8.16 (dd, 1 H), 7.65-7.75 (m, 2 H), 7.61 (dd, 1 H), 7.33- |
| | | | | | | | 7.46 (m, 2 H), 7.15-7.33 (m, 1 H), 6.95 (dd, 1 H). |
| Cpd | LC-2 | 3.46 | 376 | | 300 | DMSO- | 12.11 (s, 1H), 10.13 (s, 1H), 7.66-7.61 (m, 2H), 7.42- |
| 141 | | | | | | d6 | 7.33 (m, 3H), 7.31-7.23 (m, 1H), 7.17-7.13 (m, 1H), |
| | | | | | | | 7.11 (d, J = 8.0 Hz, 1H), 7.04 (dd, J = 7.9, 1.8 Hz, 1H), |
| | | | | | | | 6.72 (dd, J = 2.6, 1.7 Hz, 1H). |
| Cpd | LC-2 | 2.42 | 368 | 370 | 400 | DMSO- | 12.13 (s, 1H), 10.14 (s, 1H), 8.27 (d, 1H), 7.73 (t, 1H), |
| 142 | | | | | | d6 | 7.64 (d, 1H), 7.42 (t, 1H), 7.34-7-24 (m, 3H), 7.33 (d, 1H). |
| Cpd | LC-3 | 1.21 | 399 | | 400 | DMSO- | 12.11 (br. s., 1 H), 9.93 (s, 1 H), 7.56-7.70 (m, 2 H), |
| 143 | | | | | | d6 | 7.46 (t, 1 H), 7.36-7.43 (m, 3 H), 7.34 (dd, 1 H), 7.23- |
| | | | | | | | 7.29 (m, 1 H), 7.20 (dt, 1 H), 6.72 (dd, 1 H). |
| Cpd | LC-3 | 1.25 | 395 | 397 | 400 | DMSO- | 12.09 (br. s., 1 H), 9.30 (s, 1 H), 7.59-7.69 (m, 2 H), |
| 144 | | | | | | d6 | 7.57 (d, 1 H), 7.45 (dd, 1 H), 7.34-7.43 (m, 2 H), 7.13- |
| | | | | | | | 7.32 (m, 3 H), 6.75-6.84 (m, 1 H), 3.81 (s, 3 H) |
| Cpd | LC-2 | 3.57 | 367 | | 400 | DMSO- | 12.15 (br. s., 1 H), 10.22 (s, 1 H), 7.53-7.74 (m, 3 H), |
| 145 | | | | | | d6 | 7.47 (dd, 1 H), 7.32-7.44 (m, 3 H), 7.13-7.32 (m, 1 |
| | | | | | | | H), 6.77 (t, 1 H). |
| Cpd | LC-2 | 1.42 | 349 | 352 | 400 | DMSO- | 12.30 (br. s., 1 H), 11.16 (br. s., 1 H), 8.30 (d, 1 H), |
| 146 | | | | | | d6 | 7.71 (dd, 1 H), 7.59-7.69 (m, 2 H), 7.46 (d, 1 H), 7.35- |
| | | | | | | | 7.44 (m, 2 H), 7.16-7.34 (m, 1 H), 6.84 (dd, 1 H) |
| Cpd | LC-3 | 1.25 | 396 | | 400 | DMSO- | 12.16 (br. s., 1 H), 9.85 (s, 1 H), 7.79-7.92 (m, 1 H), |
| 147 | | | | | | d6 | 7.59-7.69 (m, 2 H), 7.53 (dd, 1 H), 7.32-7.49 (m, 3 |
| | | | | | | | H), 7.14-7.32 (m, 1 H), 6.82 (dd, 1 H), 3.88 (s, 3 H) |
| Cpd | LC-2 | 3.03 | 420 | 422 | 400 | DMSO- | 12.51 (brs, 1H), 10.76 (s, 1H), 8.44 (d, 1H), 7.82 (td, |
| 148 | | | | | | d6 | 1H), 7.73 (td, 1H), 7.61 (brs, 1H), 7.50 (q, 3H), 7.41- |
| | | | | | | | 7.37 (m, 1H), 6.99 (brs, 1H |
| Cpd | LC-2 | 2.24 | 342 | 344 | 400 | DMSO- | 12.19 (brs, s, 1H), 9.59 (s, 1H), 8.53 (d, 1H), 7.82-7.75 |
| 149 | | | | | | d6 | (m, 2H), 7.25-7.21 (m, 2H), 6.97 (s, 1H), 6.73-6.68 (m, |
| | | | | | | | 3H), 5.91 (s, 2H) |
| Cpd | LC-2 | 2.29 | 403 | 405 | 300 | DMSO- | 12.66 (s, 1H), 10.83 (s, 1H), 8.79 (d, J = 4.9 Hz, 2H), |
| 150 | | | | | | d6 | 7.73 (dd, J = 10.3, 6.6 Hz, 1H), 7.60 (dd, J = 3.3, 1.8 |
| | | | | | | | Hz, 1H), 7.51 (dd, J = 12.2, 6.3 Hz, 1H), 7.34 (m, 1H), |
| | | | | | | | 7.16 (m, 1H) |
| Cpd | LC-2 | 3.31 | 347 | 349 | 300 | DMSO- | 11.27 (s, 1H), 9.59 (s, 1H), 7.27-7.18 (m, 7H), 7.09 (dd, |
| 151 | | | | | | d6 | J = 3.2, 2.2 Hz, 1H), 7.05-6.97 (m, 1H), 6.26-6.23 (m, |
| | | | | | | | 1H), 3.34 (s, 2H). |
| Cpd | LC-2 | 3.09 | 380 | 382 | 300 | DMSO- | 11.50 (s, 1H), 10.82 (s, 1H), 8.40 (d, J = 2.5 Hz, 1H), |
| 152 | | | | | | d6 | 7.74 (d, J = 8.6 Hz, 1H), 7.64 (dd, J = 8.6, 2.5 Hz, 1H), |
| | | | | | | | 7.50 (dd, J = 3.2, 2.2 Hz, 1H), 7.25-7.12 (m, 5H), 6.40 |
| | | | | | | | (m, 1H), 3.89 (m, 2H). |
| Cpd | LC-2 | 3.67 | 363 | 365 | 300 | DMSO- | 11.31 (s, 1H), 9.82 (s, 1H), 7.40 (dd, J = 10.3, 2.4 Hz, |
| 153 | | | | | | d6 | 1H), 7.36-7.23 (m, 3H), 7.22-7.15 (m, 5H), 6.27 (m, |
| | | | | | | | 1H), 3.83 (m, 2H) |
| Cpc | LC-2 | 3.39 | 396 | 398 | 300 | DMSO- | 12.28 (s, 1H), 9.38 (s, 1H), 8.53 (m, 1H), 7.86-7.71 (m, |
| 154 | | | | | | d6 | 2H), 7.61-7.51 (m, 1H), 7.38 (dd, J = 3.1, 1.7 Hz, 1H), |
| | | | | | | | 7.30-7.19 (m, 3H), 7.07 (m, 1H), 3.81 (s, 3H). |
| Cpd | LC-2 | 3.19 | 416 | 418 | 300 | DMSO- | 12.26 (s, 1H), 10.69 (s, 1H), 8.45 (dd, J = 4.8, 1.7 Hz, |
| 155 | | | | | | d6 | 1H), 7.78-7.66 (m, 2H), 7.58-7.42 (m, 2H), 7.23 (dd, |
| | | | | | | | J = 7.6, 4.7 Hz, 1H), 6.84 (dd, J = 2.6, 1.6 Hz, 1H), 2.43 |
| | | | | | | | (s, 3H) |
| Cpc | LC-2 | 2.18 | 405 | 407 | 400 | DMSO- | 12.27 (s, 1H), 10.73 (s, 1H), 7.72 (dd, J = 10.3, 6.6 Hz, |
| 156 | | | | | | d6 | 1H), 7.54-7.45 (m, 2H), 7.23 (d, J = 1.2 Hz, 1H), 6.95 |
| | | | | | | | (d, J = 1.2 Hz, 1H), 6.72 (d, J = 1.6 Hz, 1H), 3.72 (s, 3H) |
| Cpc | LC-2 | 3.32 | 400 | 402 | 300 | DMSO- | 12.30 (s, 1H), 9.98 (s, 1H), 8.54 (m, 1H), 7.87-7.72 |
| 157 | | | | | | d6 | (m, 2H), 7.51-7.35 (m, 2H), 7.30-7.15 (m, 3H), 6.99 |
| | | | | | | | (dd, J = 2.6, 1.7 Hz, 1H). |
| Cpd | LC-2 | 3.81 | 415 | | 400 | DMSO- | 11.51 (s, 1H), 10.76 (s, 1H), 7.67 (dd, J = 10.3, 6.5 Hz, |
| 158 | | | | | | d6 | 1H), 7.53-7.47 (m, 1H), 7.36 (dd, J = 12.4, 6.3 Hz, 1H), |
| | | | | | | | 7.22 (m, 2H), 7.15 (d, J = 7.0 Hz, 3H), 6.41 (s, 1H), |
| | | | | | | | 3.90 (s, 2H). |
| Cpd | LC-2 | 2.92 | 388 | | 300 | DMSO- | 12.78 (s, 1H), 10.78 (s, 1H), 7.85 (dd, J = 10.6, 1.8 Hz, |
| 159 | | | | | | d6 | 1H), 7.61 (m, 1H), 7.47 (m, 1H), 7.31-7.08 (m, 5H), |
| | | | | | | | 5.92 (d, J = 2.2 Hz, 1H), 3.88 (s, 2H). |
| Cpc | LC-2 | 2.79 | 352 | 354 | 300 | DMSO- | 12.34 (s, 1H), 9.75 (s, 1H), 8.44 (d, J = 4.6 Hz, 1H), |
| 160 | | | | | | d6 | 7.81 (dd, J = 11.2, 8.1 Hz, 1H), 7.39(m, 1H), 7.26(m, |
| | | | | | | | 3H), 7.09-6.97 (m, 1H), 6.87 (s, 1H). |
| Cpc | LC-2 | 3.31 | 366 | | 300 | DMSO- | 11.40 (s, 1H), 9.20 (s, 1H), 7.47-7.34 (m, 3H), 7.33- |
| 161 | | | | | | d6 | 7.10 (m, 6H), 6.38 (d, J = 2.4 Hz, 1H), 3.89 (s, 2H), |
| | | | | | | | 3.77 (s, 3H) |
| Cpd | LC-2 | 2.92 | 336 | | 300 | DMSO- | 11.45 (s, 1H), 10.70 (s, 1H), 7.71-7.61 (m, 2H), 7.45 |
| 162 | | | | | | d6 | (dd, J = 3.2, 2.2 Hz, 1H), 7.24-7.12 (m, 7H), 6.36 (d, J = |
| | | | | | | | 2.4 Hz, 1H), 3.34 (s, 2H) |
| Cpc | LC-2 | 3.22 | 329 | 331 | 300 | DMSO- | 11.26 (s, 1H), 9.66 (s, 1H), 7.34 (dd, J = 7.6, 2.0 Hz, |
| 163 | | | | | | d6 | 1H), 7.28-7.22 (m, 2H), 7.22-7.13 (m, 7H), 6.24 (m, |
| | | | | | | | 1H), 3.34 (s, 2H). |
| Cpd | LC-2 | 1.56 | 351 | 353 | 400 | DMSO- | 12.26 (brs, 1H), 10.84 (brs, 1H), 8.53-8.52 (m, 1H), |
| 164 | | | | | | d6 | 8.22 (d, 1H), 8.0 (dd, 1H), 7.79 (d, 2H), 7.43 (brs, 1H), |
| | | | | | | | 7.26-7.22 (m, 1H), 7.13 (brs, 1H). |
| Cpd | LC-2 | 2.8 | 366 | 368 | 400 | DMSO- | 11.98 (brs, 1H), 8.84 (s, 1H), 7.61 (d, 2H), 7.39-7.32 |
| 165 | | | | | | d6 | (m, 3H), 7.30 (brs, 1H), 7.23 (t, 1H), 6.92 (brs, 1H), |
| | | | | | | | 6.86 (d, 1H), 6.70 (brs, 1H), 3.92 (s, 2H), 3.67 (s, 3H). |
| Cpc | LC-2 | 1.27 | 341 | | 400 | DMSO- | 11.91 (s, 1H), 11.52 (brs, 1H), 7.92 (brs, 1H), 7.54 (d, |
| 166 | | | | | | d6 | 2H), 7.47 (brs, 1H), 7.37 (t, 2H), 7.21 (t, 1H), 6.90 (brs, 2H). |
| Cpd | LC-2 | 2.35 | | 409 | 400 | DMSO- | 12.1 (s, 1H), 9.86 (brs, 1H), 8.52 (d, 1H), 7.93 (s, 1H), |
| 167 | | | | | | d6 | 7.81-7.78 (m, 2H), 7.54 (d, 1H), 7.43 (s, 1H), 7.24-7.2 |
| | | | | | | | (m, 1H), 7.14 (s, 1H), 3.81 (s, 3H) |
| Cpc | LC-2 | 2.52 | 396 | 398 | 400 | DMSO- | 12.08 (s, 1H), 10.81 (brs, 1H), 8.28 (d, 1H), 8.06 (dd, |
| 168 | | | | | | d6 | 1H), 7.65 (d, 2H), 7.49 (brs, 1H), 7.39 (t, 2H), 7.25 (t, |
| | | | | | | | 1H), 6.87 (brs, 2H). |
| Cpc | LC-2 | 1.75 | 355 | 357 | 400 | DMSO- | 12.06 (s, 1H), 10.70 (s, 1H), 8.11 (s, 1H), 7.70-7.63 (m, |
| 169 | | | | | | d6 | 3H), 7.50 (s, 1H), 7.38 (t, 2H), 7.24 (t, 1H), 6.87 (s, 1H), |
| | | | | | | | 4.00 (s, 2H) |
| Cpd | LC-2 | 3.01 | 432 | 434 | 400 | DMSO- | 12.19 (s, 1H), 10.74 (s, 1H), 8.17 (dd, J = 4.6, 1.3 Hz, |
| 170 | | | | | | d6 | 1H), 7.72 (dd, J = 10.3, 6.6 Hz, 1H), 7.58-7.46 (m, |
| | | | | | | | 3H), 7.30 (dd, J = 8.4, 4.6 Hz, 1H), 7.12 (m, 1H), 3.95 |
| | | | | | | | (s, 3H). |
| Cpd | LC-2 | 2.82 | 372 | | 300 | DMSO- | 11.53 (s, 1H), 10.55 (s, 1H), 7.75 (dd, J = 10.8, 1.7 Hz, |
| 171 | | | | | | d6 | 1H), 7.60-7.45 (m, 2H), 7.42 (dd, J = 3.2, 2.2 Hz, 1H), |
| | | | | | | | 7.25(td, J = 7.9, 6.2 Hz, 1H), 7.04-6.85 (m, 3H), 6.54 (t, |
| | | | | | | | J = 2.3 Hz, 1H), 3.93 (s, 2H). |
| Cpd | LC-2 | 2.97 | 412 | 414 | 300 | DMSO- | 12.34 (s, 1H), 10.26 (s, 1H), 8.54 (m, 1H), 7.87-7.77 |
| 172 | | | | | | d6 | (m, 2H), 7.71 (dd, J = 9.7, 6.4 Hz, 1H), 7.41-7.31 (m, |
| | | | | | | | 2H), 7.25 (m, 1H), 7.02 (m, 1H) |
| Cpc | LC-2 | 2.9 | 348 | 350 | 300 | DMSO- | 12.28 (s, 1H), 9.92 (s, 1H), 8.53 (m, 1H), 7.86-7.72 (m, |
| 173 | | | | | | d6 | 2H), 7.32-7.19 (m, 2H), 7.11 (m, 2H), 6.99 (m, 1H), |
| | | | | | | | 2.14 (d, J = 2.1 Hz, 3H). |
| Cpd | LC-2 | 3.66 | 410 | 412 | 300 | DMSO- | 12.35 (s, 1H), 10.25 (s, 1H), 8.54 (m, 1H), 7.87-7.76 |
| 174 | | | | | | d6 | (m, 2H), 7.57-7.33 (m, 8H), 7.37-7.18 (m, 2H), 7.07 |
| | | | | | | | (m, 1H). |
| Cpd | LC-2 | 3.1 | 396 | 398 | 400 | DMSO- | 12.08 (brs, 1H), 10.27 (brs, 1H), 8.22 (s, 1H), 7.64 (d, |
| 175 | | | | | | d6 | 2H), 7.57 (d, 2H), 7.38 (t, 2H), 7.24 (t, 1H), 6.93 (s, 1H), |
| | | | | | | | 3.88 (s, 3H). |
| Cpd | LC-2 | 2.2 | 402 | 404 | 400 | DMSO- | 12.08 (brs, 1H), 11.38 (brs, 1H), 8.48 (s, 1H), 8.09 (d, |
| 176 | | | | | | d6 | 1H), 7.74 (t, 1H), 7.62 (s, 1H), 7.32-7.23 (m, 3H), 6.93 |
| | | | | | | | (d, 1H). |
| Cpd | LC-2 | 2.87 | 372 | | 300 | DMSO- | 11.49 (s, 1H), 10.53 (s, 1H), 7.77 (dd, J = 10.7, 1.8 Hz, |
| 177 | | | | | | d6 | 1H), 7.58-7.48 (m, 2H), 7.39 (t, J = 2.7 Hz, 1H), |
| | | | | | | | 7.17(dd, J = 8.4, 5.5 Hz, 2H), 7.03 (t, J = 8.7Hz, 2H), |
| | | | | | | | 6.44 (d, J = 2.5 Hz, 1H), 3.88 (s, 2H). |
| Cpd | LC-2 | 3.32 | 374 | 376 | 300 | DMSO- | 12.27 (s, 1H), 9.89 (s, 1H), 8.53 (m, 1H), 7.79 (d, J = |
| 178 | | | | | | d6 | 7.0 Hz, 2H), 7.31-7.19 (m, 2H), 7.07 (dd, J = 11.3, 6.7 |
| | | | | | | | Hz, 1H), 6.98 (s, 1H), 6.82 (dd, J = 11.3, 7.0 Hz, 1H), |
| | | | | | | | 1.94(m, 1H), 0.92 (m, 2H), 0.68 (m, 2H). |
| Cpd | LC-2 | 2.64 | 352 | 354 | 300 | DMSO- | 12.30 (s, 1H), 10.00 (s, 1H), 8.53 (m, 1H), 7.86-7.73 |
| 179 | | | | | | d6 | (m, 2H), 7.55 (m, 1H), 7.44-7.32 (m, 1H), 7.32-7.26 (m, |
| | | | | | | | 1H), 7.26-7.19 (m, 1H), 6.98 (dd, J = 2.5, 1.7 Hz, 1H) |
| Cpd | LC-2 | 3.72 | 449 | 451 | 400 | DMSO- | 11.77 (s, 1H), 10.74 (s, 1H), 7.74 (dd, J = 10.3, 6.5 Hz, |
| 180 | | | | | | d6 | 1H), 7.58-7.47 (m, 2H), 7.33 (m, 1H), 7.00-6.88 (m, |
| | | | | | | | 2H), 6.72 (m, 1H), 3.86 (s, 3H). |
| Cpd | LC-2 | 1.28 | 350 | 352 | 300 | DMSO- | 12.27 (s, 1H), 9.92 (s, 1H), 8.53 (m, 1H), 7.86-7.71 (m, |
| 181 | | | | | | d6 | 2H), 7.46-7.30 (m, 2H), 7.24 (m, 3H), 6.98 (d, J = 1.9 |
| | | | | | | | Hz, 1H). |
| Cpd | LC-2 | 2.83 | 372 | | 300 | DMSO- | 11.49 (s, 1H), 10.59 (s, 1H), 7.78 (d, J = 10.6 Hz, 1H), |
| 182 | | | | | | d6 | 7.56 (d, J = 6.2 Hz, 2H), 7.42 (s, 1H), 7.24 (d, J = 7.4 |
| | | | | | | | Hz, 1H), 7.09 (m, 3H), 6.31 (s, 1H), 3.92 (s, 2H) |
| Cpd | LC-2 | 2.47 | 364 | 366 | 400 | DMSO- | 12.24 (s, 1H), 9.67 (s, 1H), 8.53 (d, J = 4.9 Hz, 1H), |
| 183 | | | | | | d6 | 7.79 (d, J = 7.4 Hz, 2H), 7.23 (dd, J = 13.5, 6.5 Hz, 2H), |
| | | | | | | | 7.09 (m, 2H), 6.96(s, 1H), 3.78 (s, 3H). |
| Cpd | LC-2 | 1.29 | 355 | 357 | 300 | DMSO- | 11.53 (s, 1H), 10.58 (s, 1H), 8.43-8.33 (m, 2H), 7.77 |
| 184 | | | | | | d6 | (dd, J = 10.8, 1.7 Hz, 1H), 7.59-7.45 (m, 3H), 7.40 (m, |
| | | | | | | | 1H), 7.24 (dd, J = 7.8, 4.7 Hz, 1H), 6.53 (s, 1H), 3.92 |
| | | | | | | | (s, 2H). |
| Cpd | LC-2 | 2.56 | 358 | 360 | 300 | DMSO- | 12.37 (s, 1H), 10.42 (s, 1H), 8.54 (m, 1H), 7.87-7.76 |
| 185 | | | | | | d6 | (m, 2H), 7.52-7.39 (m, 2H), 7.37-7.27 (m, 1H), 7.27- |
| | | | | | | | 7.18 (m, 1H), 7.05(d, J = 2.1 Hz, 1H), 4.52 (s, 1H). |
| Cpd | LC-2 | 2.6 | 384 | | 400 | DMSO- | 12.19 (s, 1H), 10.78 (br, s, 1H), 8.08 (t, 1H), 7.77 (d, |
| 186 | | | | | | d6 | 1H), 7.64 (d, 2H), 7.53 (s, 1H), 7.38 (t, 2H), 7.25 (t, 1H), |
| | | | | | | | 6.80 (s, 1H) |
| Cpd | LC-2 | 2.47 | 366 | 368 | 400 | DMSO- | 12.05(s, 1H), 10.15 (s, 1H), 8.80 (d, 1H), 8.39 (d, 1H), |
| 187 | | | | | | d6 | 7.96 (d, 1H), 7.73 (d, 1H), 7.60 (d, 2H), 7.48-7.45 (m, |
| | | | | | | | 2H), 7.36 (t, 2H), 7.23 (t, 1H), 6.79 (s, 1H) |
| Cpd | LC-2 | 3.23 | 382 | 384 | 400 | DMSO- | 12.09 (s, 1H), 9.84 (br, s, 1H), 7.85 (t, 1H), 7.69-7.33 |
| 188 | | | | | | d6 | (m, 5H), 7.29 (t, 1H), 7.25 (t, 1H), 7.01 (d, 1H), 6.70 (s, 1H) |
| Cpd | LC-2 | 3 | 420 | 422 | 400 | DMSO- | 12.38 (brs, 1H), 10.74 (s, 1 H), 8.53 (d, 1 H), 7.9-7.87 |
| 189 | | | | | | d6 | (m, 1 H), 7.78-7.69 (m, 2 H), 7.53-7.47 (m, 2 H), 7.06 |
| | | | | | | | (s, 1 H). |
| Cpc | LC-2 | 3.29 | 384 | 386 | 400 | DMSO- | 12.36 (s, 1H), 10.85 (s, 1H), 8.51 (d, 1H), 7.8-7.78 (m, |
| 190 | | | | | | d6 | 2H), 7.65-7.61 (m, 1H), 7.49-7.48 (m, 1H), 7.26-7.22 |
| | | | | | | | (m, 1H), 7.15-7.1 (m, 2H), 7.04 (t, 1H) |
| Cpd | LC-2 | 3.14 | 434 | 436 | 300 | DMSO- | 12.02 (s, 1H), 10.75 (s, 1H), 8.13-8.00 (m, 2H), 7.73 |
| 191 | | | | | | d6 | (dd, J = 10.4, 6.6 Hz, 1H), 7.64 (dd, J = 3.3, 1.8 Hz, |
| | | | | | | | 1H), 7.52 (dd, J = 12.3, 6.3 Hz, 1H), 7.06 (dd, J = 7.6, |
| | | | | | | | 4.9 Hz, 1H), 6.99 (m, 1H), 3.97 (s, 3H). |
| Cpd | LC-2 | 2.64 | 364 | 366 | 300 | DMSO- | 11.37 (s, 1H), 10.74 (s, 1H), 8.11 (d, J = 2.2 Hz, 1H), |
| 192 | | | | | | d6 | 7.94 (dd, J = 10.0, 2.2 Hz, 1H), 7.37 (dd, J = 3.2, 2.3 |
| | | | | | | | Hz, 1H), 7.26-7.08 (m, 5H), 6.33 (m, 1H), 3.94 (s, 2H) |
| Cpd | LC-2 | 3.8 | | 426 | 300 | DMSO- | 11.39 (s, 1H), 10.22 (s, 1H), 7.69 (dd, J = 9.7, 6.4 Hz, |
| 193 | | | | | | d6 | 1H), 7.34-7.13 (m, 7H), 6.33 (d, J = 2.4 Hz, 1H), 3.87 |
| | | | | | | | (s, 2H). |
| Cpd | LC-2 | 3.07 | 388 | | 300 | DMSO- | 11.48 (s, 1H), 10.61 (s, 1H), 7.83-7.72 (m, 1H), 7.63- |
| 194 | | | | | | d6 | 7.49 (m, 2H), 7.48-7.36 (m, 2H), 7.29-7.13 (m, 2H), |
| | | | | | | | 7.08 (dd, J = 7.4, 2.1Hz, 1H), 6.25-6.17 (m, 1H), 3.97 |
| | | | | | | | (s, 2H). |
| Cpd | LC-2 | 2.78 | 368 | 370 | 300 | DMSO- | 12.36 (s, 1H), 9.61 (s, 1H), 7.89-7.75 (m, 3H), 7.73- |
| 195 | | | | | | d6 | 7.60 (m, 4H), 7.55 (dd, J = 8.2, 6.7 Hz, 2H), 7.46 (d, J = |
| | | | | | | | 2.0 Hz, 1H). |
| Cpd | LC-2 | 2.42 | 346 | 348 | 400 | DMSO- | 12.23 (s, 1H), 9.77 (s, 1H), 8.53 (d, 1H), 7.81-7.74 (m, |
| 196 | | | | | | d6 | 2H), 7.25-7.22 (m, 2H), 7.09-7.04 (m, 1H), 6.97 (s, 1H), |
| | | | | | | | 6.87-6.85 (m, 1H), 6.67-6.63 (m, 1H), 3.68 (s, 3H). |
| Cpd | LC-2 | 3.33 | 436 | | 400 | DMSO- | 12.47 (brs, 1H), 10.77 (brs, 1 H), 8.57 (d, 1 H), 7.95- |
| 197 | | | | | | d6 | 7.92 (m, 1 H), 7.85 (d, 1 H), 7.38-7.69 (m, 1 H), 7.51- |
| | | | | | | | 7.46 (m, 1 H), 7.13 (d, 1 H). |
| Cpd | LC-2 | 2.38 | 397 | 399 | 400 | DMSO- | 12.19 (s, 1H), 10.3 (s, 1H), 8.53 (d, 1H), 8.17 (s, 1H), |
| 198 | | | | | | d6 | 7.79-7.78 (m, 2H), 7.49-7.47 (m, 2H), 7.23-7.21 (m, |
| | | | | | | | 1H), 7.17 (s, 1H), 3.87 (s, 3H). |
| Cpd | LC-2 | 1.82 | 384 | 386 | 400 | DMSO- | 12.26 (s, 1H), 11.34 (br, s, 1H), 8.59 (s, 1H), 7.85 (d, |
| 199 | | | | | | d6 | 1H), 7.64 (d, 3H), 7.38 (t, 2H), 7.25 (t, 1H), 6.81 (s, 1H) |
| Cpd | LC-2 | 1.73 | 358 | 360 | 400 | DMSO- | 12.32 (s, 1H), 10.35 (s, 1H), 8.53 (d, 1H), 7.82-7.66 (m, |
| 200 | | | | | | d6 | 4H), 7.6-7.56 (m, 1H), 7.38 (s, 1H), 7.26-7.22 (m, 1H), |
| | | | | | | | 7.0 (s, 1H), 2.5 (s, 3H) |
| Cpd | LC-2 | 4.09 | | 358 | 400 | DMSO- | 12.19 (s, 1H), 10.8 (brs, 1H), 8.53-8.51 (m, 1H), 8.07 |
| 201 | | | | | | d6 | (brs, 1H), 7.79-7.76 (m, 2H), 7.66-7.64 (m, 1H), 7.42 |
| | | | | | | | (brs, 1H), 7.26-7.21 (m, 1H), 7.13 (brs, 1H), 3.99 (s, 1H) |
| Cpd | LC-2 | 2.94 | 368 | | 400 | DMSO- | 12.29 (s, 1H), 10.23 (s, 1H), 8.53 (d, 1H), 7.79-7.78 (m, |
| 202 | | | | | | d6 | 2H), 7.62-7.57 (m, 1H), 7.38-7.34 (m, 2H), 7.26-7.24 |
| | | | | | | | (m, 1H), 7.0 (s, 1H). |
| Cpd | LC-2 | 2.77 | 388 | 390 | 400 | DMSO- | 12.28 (s, 1H), 10.33 (s, 1H), 8.52 (d, 1H), 7.81-7.75 (m, |
| 203 | | | | | | d6 | 2H), 7.71-7.69 (m, 1H), 7.6-6.56 (m, 2H), 7.36 (s, 1H), |
| | | | | | | | 7.25-7.22 (m, 1H), 7.02 (bs, 1H), 4.25 (q, 2H), 1.25 (t, 3H). |
| Cpd | LC-2 | 3.29 | 436 | 438 | 400 | DMSO- | 12.39 (brs, 1H), 10.73 (brs, 1 H), 8.56 (dd, 1 H), 8.0 |
| 204 | | | | | | d6 | (dd, 1 H), 7.72-7.68 (m, 1 H), 7.53 (brs, 1H), 7.51-7.46 |
| | | | | | | | (m, 1 H), 7.35-70.32 9m, 2 H). |
| Cpd | LC-2 | 2.85 | 400 | 402 | 300 | DMSO- | 12.33 (s, 1H), 10.14 (s, 1H), 8.54 (m, 1H), 7.86-7.76 |
| 205 | | | | | | d6 | (m, 2H), 7.41 (dd, J = 9.7, 6.5 Hz, 1H), 7.39-7.31 (m, |
| | | | | | | | 2H), 7.29-7.19 (m, 2H), 7.05-6.99 (m, 1H). |
| Cpd | LC-2 | 3.16 | 388 | | 300 | DMSO- | 11.55 (s, 1H), 10.55 (s, 1H), 7.75 (dd, J = 10.7, 1.8 Hz, |
| 206 | | | | | | d6 | 1H), 7.56-7.45 (m, 2H), 7.45-7.40 (m, 1H), 7.28-7.17(m, |
| | | | | | | | 2H), 7.15-7.07 (m, 2H), 6.57 (m, 1H), 3.92 (s, 2H). |
| Cpd | LC-2 | 1.74 | 370 | | 400 | DMSO- | 11.53 (s, 1H), 10.16 (s, 1H), 7.78 (d, J = 10.7 Hz, 1H), |
| 207 | | | | | | d6 | 7.53 (d, J = 4.9 Hz, 2H), 7.40 (m, 1H), 7.33 (d, J = 7.5 |
| | | | | | | | Hz, 2H), 7.25 (m, 2H), 7.21-7.15 (m, 1H), 6.47 (d, J = |
| | | | | | | | 2.5 Hz, 1H), 5.96 (d, J = 3.6 Hz, 1H), 5.61 (d, J = 4.8 |
| | | | | | | | Hz, 1H). |
| Cpd | LC-2 | 3.25 | 388 | | 300 | DMSO- | 11.51 (s, 1H), 10.52 (s, 1H), 7.75 (dd, J = 10.8, 1.6 Hz, |
| 208 | | | | | | d6 | 1H), 7.58-7.42 (m, 2H), 7.42-7.34 (m, 1H), 7.26 (d, J = |
| | | | | | | | 8.4 Hz, 2H), 7.15 (d, J = 8.4Hz, 2H), 6.48 (s, 1H), 3.89 |
| | | | | | | | (s, 2H). |
| Cpd | LC-2 | 2.81 | 385 | | 300 | DMSO- | 11.44 (s, 1H), 10.50 (s, 1H), 7.78 (dd, J = 10.7, 1.7 Hz, |
| 209 | | | | | | d6 | 1H), 7.61-7.46 (m, 2H), 7.38 (dd, J = 3.2, 2.2 Hz, 1H), |
| | | | | | | | 7.12-7.01 (m, 2H), 6.84-6.73 (m, 2H), 6.35 (m, 1H), |
| | | | | | | | 3.81 (s, 2H), 3.71 (s, 3H). |
| Cpd | LC-2 | 2.83 | 384 | | 400 | DMSO- | 11.47 (s, 1H), 10.52 (s, 1H), 7.76 (dd, J = 10.7, 1.8 Hz, |
| 210 | | | | | | d6 | 1H), 7.59-7.46 (m, 2H), 7.39 (m, 1H), 7.13(m, 1H), |
| | | | | | | | 6.75-6.66 (m, 3H), 6.43 (m, 1H), 3.86 (s, 2H), 3.68 (d, J = |
| | | | | | | | 2.3 Hz, 3H) |
| Cpd | LC-2 | 2.98 | 384 | | 300 | DMSO- | 11.39 (s, 1H), 10.55 (s, 1H), 7.84-7.74 (m, 1H), 7.63- |
| 211 | | | | | | d6 | 7.49 (m, 2H), 7.39 (dd, J = 3.2, 2.2 Hz, 1H), 7.17 (m, |
| | | | | | | | 1H), 6.99-6.88 (m, 2H), 6.77 (m, 1H), 6.22 (d, J = 2.4 |
| | | | | | | | Hz, 1H), 3.83 (s, 2H), 3.72 (s, 3H). |
| Cpd | LC-2 | 1.93 | 360 | | 300 | DMSO- | 12.74 (s, 1H), 10.79 (s, 1H), 7.88 (dd, J = 10.5, 1.8 Hz, |
| 212 | | | | | | d6 | 1H), 7.78 (d, J = 3.1 Hz, 1H), 7.66 (d, J = 8.4 Hz, 1H), |
| | | | | | | | 7.54 (m, 1H), 7.02 (s, 1H), 3.54-3.43 (m, 1H), 1.79 (s, |
| | | | | | | | 2H), 1.72-1.53 (m, 6H). |
| Cpd | LC-2 | 2.8 | 315 | | 400 | DMSO- | 11.38 (S, 1H), 9.44 (brs, 1H), 7.57-7.5 (m, 1H), 7.29- |
| 213 | | | | | | d6 | 7.23 (m, 1H), 7.05 (s, 1H), 5.89 (s, 1H), 1.82-1.75 (m, |
| | | | | | | | 1H), 0.83-0.79 (M, 2H), 0.56-0.53 (m, 2H) |
| Cpd | LC-2 | 3.31 | 436 | | 400 | DMSO- | 12.49 (brs, 1H), 10.77 (brs, 1 H), 8.5 (d, 1 H), 8.02 (brs, |
| 214 | | | | | | d6 | 1 H), 7.74-7.70 (m, 1 h), 7.57 (s, 1 H), 7.51-7.47 (m, 1 |
| | | | | | | | H), 7.38 9dd, 1 H), 7.23 (s, 1 H) |
| Cpd | LC-2 | 3.33 | 436 | | 400 | DMSO- | 12.41 (brs, 1H), 10.75 (brs, 1 H), 7.88-7.79 (m, 2 H), |
| 215 | | | | | | d6 | 7.73-7.69 (m, 1 H), 7.56 (s, 1 H), 7.50-7.46 (m, 1 H), |
| | | | | | | | 7.34 (d, 1 H), 7.14 (s, 1 H). |
| Cpd | LC-2 | 1.09 | 340 | 342 | 400 | DMSO- | 12.11 (br. s., 1 H), 10.97 (br. s., 1 H), 8.27 (s, 1 H), |
| 216 | | | | | | d6 | 7.82 (dd, 1 H), 7.66 (d, 2 H), 7.48-7.60 (m, 1 H), 7.39 |
| | | | | | | | (t, 2 H), 7.16-7.30 (m, 1 H), 6.90 (s, 1 H), 4.38 (s, 1 H) |
| Cpd | LC-2 | 2.7 | 358 | | 400 | DMSO- | 11.77 (s, 1H), 10.38 (s, 1H), 7.68 (d, 1H), 7.5-7.43 (m, |
| 217 | | | | | | d6 | 4H), 7.39-7.35 (m, 1H), 7.12-7.07 (m, 2H), 7.03-7.01 |
| | | | | | | | (m, 1H). |
| Cpd | LC-2 | 3.07 | 374 | | 400 | DMSO- | 11.83 (s, 1H), 10.47 (s, 1H), 7.68 (d, 1H), 7.48-7.46 (m, |
| 218 | | | | | | d6 | 4H), 7.39-7.3 (m, 3H), 7.03-7.01 (m, 1H). |
| Cpd | LC-2 | 2.98 | 354 | | 400 | DMSO- | 11.7 (s, 1H), 10.34 (s, 1H), 7.66 (d, 1H), 7.48-7.34 (m, |
| 219 | | | | | | d6 | 5H), 7.07 (d, 2H), 6.97 (s, 1H), 2.29 (s, 3H). |
| Cpd | LC-2 | 2.58 | 370 | | 400 | DMSO- | 11.77 (s, 1H), 10.35 (s, 1H), 7.68 (d, 1H), 7.45-7.34 (m, |
| 220 | | | | | | d6 | 3H), 7.2-7.16 (m, 1H), 7.03-7.01 (m, 3H), 6.79-6.77 (m, 1H). |
| Cpd | LC-2 | 2.94 | 354 | | 400 | DMSO- | 11.7 (s, 1H), 10.37 (s, 1H), 7.69-7.66 (m, 1H), 7.47- |
| 221 | | | | | | d6 | 7.35 (m, 3H), 7.39-7.35 (m, 1H), 7.26-7.14 (m, 3H), |
| | | | | | | | 7.04-6.98 (m, 2H), 2.67 (s, 3H) |
| Cpd | LC-2 | 3.81 | 374 | | 400 | DMSO- | 11.79 (s, 1H), 10.17 (s, 1H), 7.7 (d, 1H), 7.48-7.46 (m, |
| 222 | | | | | | d6 | 1H), 7.44-7.41 (m, 1H), 7.36-7.32 (m, 2H), 7.3-7.22 (m, |
| | | | | | | | 3H), 6.89-6.86 (m, 1H) |
| Cpd | LC-2 | 2.66 | 370 | | 400 | DMSO- | 11.68 (s, 1H), 10.11 (s, 1H), 7.74 (d, 1H), 7.48-7.46 (m, |
| 223 | | | | | | d6 | 2H), 7.4-7.33 (m, 1H), 7.26-7.11 (m, 2H), 6.92 (d, 1H), |
| | | | | | | | 6.88-6.81 (m, 2H), 3.57 (s, 3H). |
| Cpd | LC-2 | 3.39 | 450 | | 300 | DMSO- | 11.81 (s, 1H), 10.75 (s, 1H), 8.13 (dd, J = 8.5, 5.7 Hz, |
| 224 | | | | | | d6 | 1H), 7.70 (d, J = 7.1 Hz, 1H), 7.58 (d, J = 3.0 Hz, 1H), |
| | | | | | | | 7.49 (dd, J = 12.4, 6.3 Hz, 1H), 7.09 (dd, J = 10.1, 5.7 |
| | | | | | | | Hz, 1H), 6.81 (m, 1H), 3.97 (s, 3H). |
| Cpd | LC-2 | 3.2 | 368 | | 300 | DMSO- | 11.53 (s, 1H), 10.45 (d, J = 1.6 Hz, 1H), 7.73 (dd, J = |
| 225 | | | | | | d6 | 10.8, 1.8 Hz, 1H), 7.55-7.38 (m, 2H), 7.36 (dd, J = |
| | | | | | | | 3.2, 2.2 Hz, 1H), 7.26-7.12 (m, 4H), 7.10 (ddd, J = |
| | | | | | | | 6.0, 5.0, 2.5 Hz, 1H), 6.72 (t, J = 2.4 Hz, 1H), 4.40 (q, J = |
| | | | | | | | 7.2 Hz, 1H), 1.43 (d, J = 7.2 Hz, 3H). |
| Cpd | LC-2 | 2.57 | 420 | | 300 | DMSO- | 12.41 (s, 1H), 10.78 (s, 1H), 8.96 (d, J = 10.6 Hz, 1H), |
| 226 | | | | | | d6 | 8.55-8.44 (m, 1H), 7.81-7.68 (m, 2H), 7.55-7.39 |
| | | | | | | | (m, 2H), 6.90 (s, 1H). |
| Cpd | LC-2 | 3.49 | 398 | 400 | 400 | DMSO- | 11.70 (s, 1H), 9.91 (s, 1H), 8.13 (dd, J = 8.4, 5.7 Hz, |
| 227 | | | | | | d6 | 1H), 7.46-7.29 (m, 3H), 7.23 (dd, J = 8.6, 2.5 Hz, 1H), |
| | | | | | | | 7.09 (dd, J = 10.1, 5.7 Hz, 1H), 6.76-6.70 (m, 1H), |
| | | | | | | | 3.98 (s, 3H). |
| Cpd | LC-2 | 2.94 | 374 | | 400 | DMSO- | 11.84 (s, 1H), 10.43 (s, 1H), 7.68 (d, 1H), 7.49-7.43 (m, |
| 228 | | | | | | d6 | 4H), 7.38-7.26 (m, 3H), 7.12-7.11 (m, 1H). |
| Cpc | LC-2 | 2.6 | 341 | 343 | 400 | DMSO- | 11.98 (s, 1H), 11.32 (s, 1H), 8.61 (d, 1H), 7.87-7.72 (m, |
| 229 | | | | | | d6 | 4H), 7.63-7.61 (m, 2H), 7.53 (s, 1H), 7.32-7.29 (m, 1H). |
| Cpd | LC-2 | 2.6 | 358 | | 400 | DMSO- | 11.84 (s, 1H), 10.47 (s, 1H), 7.67 (d, 1H), 7.49-7.46 (m, |
| 230 | | | | | | d6 | 2H), 7.4-7.29 (m, 4H), 7.12-7.11 (m, 1H), 7.06-7.0 (m, 1H). |
| Cpd | LC-2 | 2.56 | 358 | | 400 | DMSO- | 11.84 (s, 1H), 10.29 (s, 1H), 7.72 (d, 1H), 7.5-7.28 (m, |
| 231 | | | | | | d6 | 5H), 7.15-7.05 (m, 2H), 6.96 (s, 1H). |
| Cpd | LC-2 | 3.44 | 399 | | 400 | CDCl3 | 12.14 (d, J = 3.4 Hz, 1H), 10.09 (s, 1H), 7.69-7.61 (m, |
| 232 | | | | | | | 2H), 7.44-7.32 (m, 5H), 7.30-7.15 (m, 2H), 6.75 (dd, |
| | | | | | | | J = 2.7, 1.7 Hz, 1H). |
| Cpd | LC-2 | 3.17 | 364 | 366 | 400 | CDCl3 | 12.11 (s, 1H), 10.09 (s, 1H), 8.00 (d, J = 2.8 Hz, 1H), |
| 233 | | | | | | | 7.78-7.56 (m, 4H), 7.43-7.35 (m, 3H), 7.26 (m, 1H), |
| | | | | | | | 7.03 (d, J = 8.8 Hz, 1H), 6.72 (m, 1H). |
| Cpd | LC-2 | 2.81 | 449 | | 300 | DMSO- | 12.22 (s, 1H), 10.75 (s, 1H), 7.92-7.57 (m, 3H), 7.49 |
| 234 | | | | | | d6 | (dd, J = 12.4, 6.4 Hz, 1H), 7.34-7.13 (m, 2H), 6.79 (m, |
| | | | | | | | 1H), 5.28 (m, 1H), 4.50 (d, J = 5.5 Hz, 2H). |
| Cpd | LC-2 | 3.52 | 463 | | 300 | DMSO- | 12.20 (s, 1H), 10.75 (s, 1H), 7.75-7.60 (m, 3H), 7.46 |
| 235 | | | | | | d6 | (dd, J = 12.5, 6.4 Hz, 1H), 7.34-7.22 (m, 2H), 6.80 (m, |
| | | | | | | | 1H), 4.40 (s, 2H), 3.30 (s, 3H). |
| Cpd | LC-2 | 2.05 | 304 | | 400 | DMSO- | 11.43 (brs, 1H), 10.35 (brs, 1 H), 7.76 (d, 1H), 7.57- |
| 236 | | | | | | d6 | 7.51 (m, 2 H), 7.19 (s, 1H), 5.96 (s, 1 H), 1.81-1.74 (m, |
| | | | | | | | 1 H), 0.82-0.77 (m, 2 H), 0.57-0.54 (m, 2 H). |
| Cpd | LC-2 | 2.92 | 378 | 380 | 400 | DMSO- | 12.21 (s, 1H), 9.59 (s, 1H), 8.52 (d, 1H), 7.81-7.75 (m, |
| 237 | | | | | | d6 | 2H), 7.25-7.22 (m, 1H), 7.17 (s, 1H), 7.11-7.03 (m, 2H), |
| | | | | | | | 6.93 (s, 1H), 4.03 (g, 2H), 1.29 (t, 3H). |
| Cpd | LC-2 | 2.79 | 330 | | 400 | DMSO- | 11.59 (brs, 1H), 1.42 (brs, 1 H), 7.76 (d, 1 H), 7.52 (d, 1 |
| 238 | | | | | | d6 | H), 7.45-7.41 (m, 2 H), 6.82 (s, 1 H), 6.25 (s, 1 H), 2.53- |
| | | | | | | | 2.45 (m, 2 H), 2.4-2.38 (m, 2 H), 1.85-1.78 (m, 2 H). |
| Cpd | LC-2 | 2.92 | 382 | 384 | 400 | DMSO- | 12.3 (s, 1H), 10.28 (s, 1H), 8.52 (d, 1H), 7.82-7.77 (m, |
| 239 | | | | | | d6 | 2H), 7.37 (s, 1H), 7.27-6.91 (m, 6H). |
| Cpc | LC-2 | 3.21 | 384 | | 400 | DMSO- | 11.74 (brs, 1H), 10.54 (brs, 1 H), 7.78 (d, 1H), 7.64- |
| 240 | | | | | | d6 | 7.56 (m, 3 H), 7.42 (s, 4H), 7.22 (t, 1 H), 7.06 (d, 1 H), |
| | | | | | | | 6.96 (t, 1 H), 6.84 (s, 1 H), 4.1 (g, 2 H), 1.35 (t, 3 H). |
| Cpd | LC-2 | 3.24 | 392 | 394 | 400 | DMSO- | 12.22 (s, 1H), 9.6 (s, 1H), 8.52 (d, 1H), 7.81-7.75 (m, |
| 241 | | | | | | d6 | 2H), 7.25-7.22 (m, 1H), 7.17 (s, 1H), 7.11-7.03 (m, 2H), |
| | | | | | | | 6.93 (s, 1H), 4.6-4.54 (m, 1H), 1.22 (d, 6H). |
| Cpd | LC-2 | 2.98 | 332 | | 400 | DMSO- | 11.35 (brs, 1H), 10.42 (brs, 1 H), 7.75 (d, 1H), 7.52- |
| 242 | | | | | | d6 | 7.48 (m, 2 H), 7.29 (s, 1 H), 6.68 (s, 1 H), 3.2-3.15 (m, |
| | | | | | | | 1 H), 1.93-1.87 (m, 2 H), 1.68-1.63 (m, 2 H), 1.56, 1.52 |
| | | | | | | | (m, 2 H), 1.34-1.23 (m., 2 H). |
| Cpd | LC-2 | 2.82 | 473 | 475 | 400 | DMSO- | 12.37 (s, 1H), 10.69 (s, 1H), 7.69 (m, 1H), 7.55-7.45 |
| 243 | | | | | | d6 | (m, 2H), 7.35 (s, 1H), 7.08 (d, J = 1.3 Hz, 1H), 6.76 (s, |
| | | | | | | | 1H), 5.06 (m, 2H). |
| Cpd | LC-2 | 2.94 | 406 | | 400 | CDCl3 | 12.08 (s, 1H), 10.57 (s, 1H), 7.85-7.77 (m, 1H), 7.69 |
| 244 | | | | | | | (dd, J = 7.7, 1.8 Hz, 1H), 7.68-7.57 (m, 2H), 7.55 (dd, |
| | | | | | | | J = 3.3, 1.7 Hz, 1H), 7.47-7.19 (m, 4H), 6.82 (dd, J = |
| | | | | | | | 2.6, 1.7 Hz, 1H). |
| Cpo | LC-2 | 2.51 | 408 | | 400 | DMSO- | 11.78 (s, 1H), 10.71 (s, 1H), 7.72 (dd, J = 10.4, 6.6 Hz, |
| 245 | | | | | | d6 | 1H), 7.44 (dd, J = 12.4, 6.3 Hz, 1H), 7.25-7.14 (m, |
| | | | | | | | 1H), 6.10 (m, 1H), 3.69 (m, 2H), 2.44 (m, 2H), 2.15- |
| | | | | | | | 1.93 (m, 2H). |
| Cpd | LC-2 | 3.37 | 418 | 420 | 400 | DMSO- | 12.34 (s, 1H), 10.32 (s, 1H), 8.52 (d, 1H), 7.84-7.76 (m, |
| 246 | | | | | | d6 | 2H), 7.7-7.66 (m, 1H), 7.48-7.44 (m, 1H), 7.39 (d, 1H), |
| | | | | | | | 7.26-7.23 (m, 1H), 7.02 (s, 1H). |
| Cpd | LC-2 | 3 | 450 | 452 | 400 | DMSO- | 11.85 (s, 1H), 10.75 (s, 1H), 8.08 (d, J = 5.9 Hz, 1H), |
| 247 | | | | | | d6 | 7.79-7.70 (m, 1H), 7.60 (dd, J = 3.3, 1.8 Hz, 1H), 7.51 |
| | | | | | | | (dd, J = 12.2, 6.3 Hz, 1H), 7.19 (dd, J = 6.0, 1.7 Hz, |
| | | | | | | | 1H), 6.78 (m, 1H), 3.96 (s, 3H). |
| Cpd | LC-2 | 2.61 | 419 | 421 | 400 | DMSO- | 12.30 (s, 1H), 10.74 (s, 1H), 7.72 (dd, J = 10.3, 6.6 Hz, |
| 248 | | | | | | d6 | 1H), 7.56-7.47 (m, 2H), 7.30 (d, J = 1.3 Hz, 1H), 6.99 |
| | | | | | | | (d, J = 1.3 Hz, 1H), 6.65 (d, J = 1.7 Hz, 1H), 4.08 (m, |
| | | | | | | | 2H), 1.28 (m, 3H). |
| Cpd | LC-2 | 3.03 | 372 | | 400 | DMSO- | 11.96(bs, 1H), 10.48(brs, 1H), 7.79-7.76 (m, 1H), |
| 249 | | | | | | d6 | 7.62-7.59 (m, 2H), 7.48 (brs, 1H), 7.30-7.25 (m, 1H), |
| | | | | | | | 7.19-7.1233 (m, 2H), 6.48 (brs, 1H), 2.33 (s, 3H) |
| Cpd | LC-2 | 3.29 | 388 | | 400 | DMSO- | 12.05 (bs, 1H), 10.52 (s, 1H), 7.81 (d, 1H), 7.62-7.61 |
| 250 | | | | | | d6 | (m, 2H), 7.51 (brs, 1H), 7.42 (d, 1H), 7.37 (brs, 1H), |
| | | | | | | | 7.20 (d, 1H), 6.74 (brs, 1H), 2.31(s, 3H) |
| Cpd | LC-2 | 2.93 | 384 | | 400 | DMSO- | 11.77 (bs, 1H), 10.45 (brs, 1H), 7.80-7.78 (m, 1H), |
| 251 | | | | | | d6 | 7.64-7.60 (m, 2H), 7.40 (brs, 1H), 7.25-7.23 (d, 1H), |
| | | | | | | | 6.85-6.80 (m, 2H), 6.34 (brs, 1H), 3.75 (s, 3H), 2.24 |
| | | | | | | | (s, 3H). |
| Cpd | LC-2 | 3.26 | 368 | | 400 | DMSO | 11.83 (bs, 1H), 10.45 (brs, 1H), 7.81-7.79 (m, 1H), |
| 252 | | | | | | d6 | 7.64-7.61 (m, 2H), 7.43 (brs, 1H), 7.17-7.10 (m, 3H), |
| | | | | | | | 6.34 (brs, 1H), 2.13 (s, 3H), 2.07 (s, 3H). |
| Cpd | LC-2 | 3.35 | 388 | | 400 | DMSO- | 11.99 (bs, 1H), 10.48 (brs, 1H), 7.83-7.80 (m, 1H), |
| 253 | | | | | | d6 | 7.61 (brs, 2H), 7.54-7.43 (m, 2H), 7.27-7.22 (m, 2H), |
| | | | | | | | 6.45 (brs, 1H), 2.38 (s, 3H). |
| Cpd | LC-2 | 3.26 | 388 | | 400 | DMSO | 12.07 (bs, 1H), 10.50 (s, 1H), 7.81 (d, 1H), 7.64-7.60 |
| 254 | | | | | | d6 | (m, 2H), 7.53-7.52 (m, 1H), 7.41-7.38 (m, 2H), 7.15 |
| | | | | | | | (dd, 1H), 6.77-6.76 (brs, 1H), 3.32 (s, 3H). |
| Cpd | LC-2 | 3.04 | 372 | | 400 | DMSO- | 11.86 (bs, 1H), 10.46 (brs, 1H), 7.78-7.75 (m, 1H), |
| 255 | | | | | | d6 | 7.62-7.58 (m, 2H), 7.44 (brs, 1H), 7.37-7.33 (m, 1H), |
| | | | | | | | 7.16-7.14 (m, 1H), 7.10-7.06 (m, 1H), 6.42 (brs, 1H), |
| | | | | | | | 2.28 (s, 3H). |
| Cpd | LC-2 | 2.95 | 392 | | 400 | DMSO- | 12.17 (bs, 1H), 10.53 (brs, 1H), 7.80 (d, 1H), 7.63-7.57 |
| 256 | | | | | | d6 | (m, 4H), 7.48 (dd, 1H), 7.234-7.19 (m, 1H), 6.91 (brs, 1H). |
| Cpd | LC-2 | 3.01 | 404 | | 400 | DMSO- | 12.13 (bs, 1H), 10.52 (s, 1H), 7.82 (d, 1H), 7.6-7.56 (m, |
| 257 | | | | | | d6 | 3H), 7.43-7.41 (d, 1H), 7.12-7.11 (d, 1H), 6.92 (dd, 1H) |
| | | | | | | | 6.84 (brs, 1H), 3.79 (s, 3H) |
| Cpd | LC-2 | 3.01 | 4 | | 400 | DMSO- | 12.09 (bs, 1H), 10.51 (brs, 1H), 7.80 (d, 1H), 7.60-7.53 |
| 258 | | | | | | d6 | (m, 5H), 7.34-7.29 (m, 1H), 6.72 (brs, 1H). |
| Cpd | LC-2 | 2.74 | 388 | | 400 | DMSO- | 12.16 (brs, 1H), 10.50 (s, 1H), 7.80 (d, 1H), 7.63-7.58 |
| 259 | | | | | | d6 | (m, 2H), 7.53-7.53 (brs, 1H), 7.43 (d, 1H), 7.24-7.20 |
| | | | | | | | (m, 2H), 6.83 (brs, 1H), 3.89 (s, 3H). |
| Cpd | LC-2 | 2.27 | 408 | | 400 | DMSO- | 12.22 (bs, 1H), 10.53 (s, 1H), 7.82 (d, 1H), 7.67 (d, 1H), |
| 260 | | | | | | d6 | 7.61-7.56 (m, 4H), 7.41 (dd, 1H), 6.90 (brs, 1H). |
| Cpd | LC-2 | 3.39 | 408 | | 400 | DMSO- | 12.15 (bs, 1H), 10.52 (s, 1H), 7.80 (d, 1H), 7.71 (d, 1H), |
| 261 | | | | | | d6 | 7.63-7.56 (m, 4H), 7.51 (dd, 1H), 6.81 (brs, 1H). |
| Cpd | LC-2 | 3.39 | 388 | | 400 | DMSO- | 11.95 (bs, 1H), 10.46 (brs, 1H), 7.81-7.79 (m, 1H), |
| 262 | | | | | | d6 | 7.64-7.60 (m, 2H), 7.45 (brs, 1H), 7.38-7.30 (m, 3H), |
| | | | | | | | 6.48 (brs, 1H), 2.29 (s, 3H). |
| Cpd | LC-2 | 3.23 | 388 | | 400 | DMSO- | 12.04 (bs, 1H), 10.49 (s, 1H), 7.81 (d, 1H), 7.65-7.60 |
| 263 | | | | | | d6 | (m, 2H), 7.51-7.50 (m, 1H), 7.35-7.33 (m, 2H), 7.30- |
| | | | | | | | 7.27 (m, 1H), 6.71 (t, 1H), 2.38 (s, 3H). |
| Cpd | LC-2 | 3.09 | 372 | | 400 | DMSO- | 12.17 (brs, 1H), 10.54 (s, 1H), 7.79 (d, 1H), 7.63-7.59 |
| 264 | | | | | | d6 | (m, 2H), 7.51-7.44 (m, 2H), 7.40-7.38 (m, 1H), 7.28 (t, |
| | | | | | | | 1H), 6.82 (brs, 1H), 2.21 (s, 3H). |
| Cpd | LC-2 | 3.01 | 404 | | 400 | DMSO- | 11.73 (bs, 1H), 10.41 (s, 1H), 7.80 (d, 1H), 7.61-7.58 |
| 265 | | | | | | d6 | (m, 2H), 7.43-7.42 (1m, 1H), 7.39-7.34 (m, 1H), 7.13- |
| | | | | | | | 7.06 (m, 2H) 6.38-6.37 (m, 1H), 3.72 (s, 3H). |
| Cpd | LC-2 | 2.78 | 404 | | 400 | DMSO- | 12.05 (bs, 1H), 10.49 (s, 1H), 7.82-7.79 (m, 1H), 7.64- |
| 266 | | | | | | d6 | 7.59 (m, 2H), 7.51-7.50 (m, 1H), 7.35 (t, 1H), 7.13-7.09 |
| | | | | | | | (m, 2H) 6.76-6.76 (m, 1H), 3.88 (s, 3H). |
| Cpd | LC-2 | 2.92 | 392 | | 400 | DMSO- | 12.19 (bs, 1H), 10.53 (brs, 1H), 7.80 (d, 1H), 7.62-7.57 |
| 267 | | | | | | d6 | (m, 3H), 7.47-7.36 (3m, 1H), 6.85 (brs, 1H). |
| Cpd | LC-2 | 2.97 | 384 | | 400 | DMSO- | 11.92 (bs, 1H), 10.45 (brs, 1H), 7.82-7.79 (m, 1H), |
| 268 | | | | | | d6 | 7.65-7.59 (m, 2H), 7.48 (d, 1H), 7.19 (dd, 1H), 6.92 (d, |
| | | | | | | | 1H), 6.82 (dd, 1H), 6.48 (brs, 1H), 3.75 (s, 3H), 2.22 (s, 3H). |
| Cpd | LC-2 | 3.09 | 372 | | 400 | DMSO- | 12.11 (brs, 1H), 10.51 (s, 1H), 7.79 (d, 1H), 7.63-7.57 |
| 269 | | | | | | d6 | (m, 3H), 7.48 (brs, 2H), 7.15 (t, 1H), 6.73 (brs, 1H), |
| | | | | | | | 2.25 (s, 3H). |
| Cpd | LC-2 | 3.05 | 408 | | 400 | DMSO- | 11.95 (brs, 1H), 10.41 (s, 1H), 7.82-7.79 (m, 1H), 7.62- |
| 270 | | | | | | d6 | 7.54 (m, 4H), 7.50-7.43 (m, 2H), 6.34 (brs, 1H). |
| Cpd | LC-2 | 3.62 | 413 | | 300 | DMSO- | 11.37 (s, 1H), 10.06 (s, 1H), 7.48-6.90 (m, 9H), 6.31 |
| 271 | | | | | | d6 | (m, 1H), 3.86 (s, 2H), |
| Cpd | LC-2 | 3.38 | 407 | | 300 | DMSO- | 12.27 (s, 1H), 10.71 (s, 1H), 7.73 (dd, J = 10.4, 6.6 Hz, |
| 272 | | | | | | d6 | 1H), 7.62 (dd, J = 3.1, 1.7 Hz, 1H), 7.55-7.41 (m, 2H), |
| | | | | | | | 7.34 (dd, J = 3.6, 1.2 Hz, 1H), 7.07 (dd, J = 5.1, 3.6 Hz, |
| | | | | | | | 1H), 6.57 (m, 1H). |
| Cpd | LC-2 | 3.43 | 407 | | 300 | DMSO- | 12.14 (s, 1H), 10.71 (s, 1H), 7.77-7.65 (m, 2H), 7.59 |
| 273 | | | | | | d6 | (m, 2H), 7.55-7.41 (m, 2H), 6.70 (m, 1H). |
| Cpd | LC-2 | 3.73 | 421 | | 300 | DMSO- | 12.17 (s, 1H), 10.69 (s, 1H), 7.72 (dd, J = 10.3, 6.6 Hz, |
| 274 | | | | | | d6 | 1H), 7.58 (dd, J = 3.1, 1.7 Hz, 1H), 7.48 (dd, J = 12.3, |
| | | | | | | | 6.3 Hz, 1H), 7.11 (d, J = 3.6 Hz, 1H), 6.74 (dd, J = 3.5, |
| | | | | | | | 1.4 Hz, 1H), 6.46 (m, 1H), 2.42 (s, 3H). |
| Cpd | LC-2 | 3.18 | 391 | | 300 | DMSO- | 12.28 (s, 1H), 10.75 (s, 1H), 7.78-7.64 (m, 2H), 7.61 |
| 275 | | | | | | d6 | (dd, J = 3.1, 1.7 Hz, 1H), 7.48 (dd, J = 12.2, 6.3 Hz, |
| | | | | | | | 1H), 6.70 (dd, J = 3.4, 0.8 Hz, 1H), 6.57 (m, 2H). |
| Cpd | LC-2 | 3.18 | 391 | | 300 | DMSO- | 12.04 (s, 1H), 10.71 (s, 1H), 8.00 (s, 1H), 7.77-7.65 |
| 276 | | | | | | d6 | (m, 2H), 7.59 (dd, J = 3.0, 1.7 Hz, 1H), 7.47 (dd, J = |
| | | | | | | | 12.3, 6.3 Hz, 1H), 6.84 (d, J = 1.9 Hz, 1H), 6.60 (m, 1H). |
| Cpd | LC-2 | 2.19 | 405 | 407 | 300 | MeOD- | 7.69 (s, 1H), 7.59 (dd, J = 12.1, 6.4 Hz, 1H), 7.53- |
| 277 | | | | | | d4 | 7.40 (m, 2H), 7.12 (d, J = 1.2 Hz, 1H), 6.63 (d, J = 1.7 |
| | | | | | | | Hz, 1H), 3.70 (s, 3H). |
| Cpd | LC-2 | 2.86 | 408 | | 300 | DMSO- | 12.54 (s, 1H), 10.77 (s, 1H), 9.10 (d, J = 4.7 Hz, 1H), |
| 278 | | | | | | d6 | 7.79 (d, J = 4.7 Hz, 1H), 7.72 (dd, J = 10.3, 6.6 Hz, 1H), |
| | | | | | | | 7.60 (dd, J = 3.2, 1.7 Hz, 1H), 7.50 (dd, J = 12.2, 6.3 |
| | | | | | | | Hz, 1H), 7.03 (m, 1H). |
| Cpd | LC-2 | 3.33 | 416 | 418 | 400 | DMSO- | 12.24 (s, 1H), 10.77 (s, 1H), 7.78-7.65 (m, 2H), 7.59 |
| 279 | | | | | | d6 | (d, J = 7.8 Hz, 1H), 7.56-7.46 (m, 2H), 7.12 (d, J = 7.5 |
| | | | | | | | Hz, 1H), 7.06 (s, 1H), 2.50 (s, 3H). |
| Cpd | LC-2 | 3.24 | 416 | 418 | 300 | DMSO- | 12.36 (s, 1H), 10.76 (s, 1H), 8.42-8.35 (m, 1H), 7.77- |
| 280 | | | | | | d6 | 7.67 (m, 2H), 7.63 (dd, J = 8.1, 2.2 Hz, 1H), 7.56-7.44 |
| | | | | | | | (m, 2H), 7.03 (m, 1H), 2.29 (s, 3H). |
| Cpd | LC-2 | 3.57 | 432 | 434 | 300 | DMSO- | 12.17 (s, 1H), 10.74 (s, 1H), 7.77-7.64 (m, 2H), 7.60 |
| 281 | | | | | | d6 | (dd, J = 3.2, 1.7 Hz, 1H), 7.50 (dd, J = 12.3, 6.3 Hz, |
| | | | | | | | 1H), 7.36 (d, J = 7.4 Hz, 1H), 7.04 (dd, J = 2.6, 1.7 Hz, |
| | | | | | | | 1H), 6.66 (d, J = 8.2 Hz, 1H), 3.93 (s, 3H). |
| Cpd | LC-2 | 3.49 | 470 | 472 | 400 | DMSO- | 12.51 (s, 1H), 10.77 (s, 1H), 8.87 (dd, J = 4.7, 1.6 Hz, |
| 282 | | | | | | d6 | 1H), 8.26 (dd, J = 8.1, 1.6 Hz, 1H), 7.74 (dd, J = 10.2, |
| | | | | | | | 6.6 Hz, 1H), 7.62 (dd, J = 3.3, 1.6 Hz, 1H), 7.59-7.46 |
| | | | | | | | (m, 2H), 6.86 (m, 1H). |
| Cpd | LC-2 | 3.03 | 384 | | 400 | MeOD- | 7.76 (t, 1H), 7.50-7.46 (m, 2H), 7.34 (d, 1H), 7.15 (t, |
| 283 | | | | | | d4 | 1H), 6.86 (dd, 2H), 6.37 (d, 1H), 3.82 (s, 3H), 2.09 (s, 3H) |
| Cpd | LC-2 | 3.12 | 392 | | 400 | DMSO- | 12.25 (s, 1H), 10.54 (s, 1H), 7.91 (d, 1H), 7.80 (d, 1H), |
| 284 | | | | | | d6 | 7.65-7.54 (m, 4H), 7.43 (t, 1H), 6.89 (s, 1H) |
| Cpd | LC-2 | 3.02 | 372 | | 400 | DMSO- | 11.99 (s, 1H), 10.46 (s, 1H), 7.80 (d, 1 H), 7.64-7.60 |
| 285 | | | | | | d6 | (m, 2 H), 7.50 (s, 1 H), 7.30 (t, 1 H), 7.2 (d, 1 H), 7.06 |
| | | | | | | | (t, 1 H), 6.54 (s, 1 H), 2.27 (s, 3 H). |
| Cpd | LC-2 | 2.22 | 355 | 357 | 400 | DMSO- | 12.35 (s, 1H), 10.45 (s, 1H), 8.52 (d, 1H), 7.82-7.78 (m, |
| 286 | | | | | | d6 | 2H), 7.70 (d, 1H), 7.48 (t, 2H), 7.25-7.22 (m, 1H), 7.05 |
| | | | | | | | (s, 1H), 2.40 (s, 3H) |
| Cpd | LC-2 | 3.15 | 392 | | 400 | DMSO- | 12.29 (s, 1H), 10.57 (s, 1H), 7.80-7.73 (m, 2H), 7.58- |
| 287 | | | | | | d6 | 7.52 (m, 5H), 6.95 (s, 1H) |
| Cpd | LC-2 | 3.09 | 372 | | 400 | DMSO- | 12.21 (s, 1H), 10.54 (s, 1H), 7.80 (d, 1H), 7.60 (s, 2H), |
| 288 | | | | | | d6 | 7.52 (s, 1H), 7.34-7.29 (m, 2H), 6.92-6.87 (m, 2H), 2.32 |
| | | | | | | | (s, 3H) |
| Cpd | LC-2 | 2.84 | 392 | | 400 | DMSO- | 12.03 (s, 1H), 10.49 (s, 1H), 7.80 (d, 1H), 7.60-7.55 (m, |
| 289 | | | | | | d6 | 3H), 7.48-7.42 (m, 2H), 7.35-7.30 (m, 1H), 6.54 (s, 1H) |
| Cpd | LC-2 | 2.94 | 388 | | 400 | DMSO- | 11.94 (s, 1H), 10.51 (s, 1H), 7.79 (d, 1 H), 7.62-7.61 |
| 290 | | | | | | d6 | (m, 2 H), 7.47-7.41 (m, 2 H), 7.21-7.12 (m, 2 H), 6.88 |
| | | | | | | | (s, 1 H), 3.77 (s, 3 H). |
| Cpd | LC-2 | 2.84 | 376 | | 400 | DMSO- | 12.22 (s, 1H), 10.53 (s, 1H), 7.76-7.73 (m, 2 H), 7.63- |
| 291 | | | | | | d6 | 7.58 (m, 2 H), 7.55-7.42 (m, 3 H), 6.87 (s, 1H) |
| Cpc | LC-2 | 3.24 | 408 | | 400 | DMSO- | 12.16 (s, 1H), 10.51 (s, 1H), 7.82 (d, 1 H), 7.64-7.59 |
| 292 | | | | | | d6 | (m, 3 H), 7.56-7.55 (m, 1 H), 7.50-7.48 (m, 1 H), 7.43- |
| | | | | | | | 7.39 (m, 1 H), 6.78 (s, 1 H). |
| Cpd | LC-3 | 1.29 | 433 | | 400 | DMSO- | 12.11 (s, 1H), 10.71 (s, 1H), 7.72-7.68 (m, 1H), 7.63- |
| 293 | | | | | | d6 | 7.58 (m, 2H), 7.50-7.46 (m, 1H), 7.13-7.06 (m, 2H), |
| | | | | | | | 6.74 (br, s, 1H), 2.31 (s, 3H). |
| Cpd | LC-2 | 3.57 | 431 | | 400 | DMSO- | 12.03 (s, 1H), 10.68 (s, 1H), 7.69 (t, 1H), 7.58-7.54 (m, |
| 294 | | | | | | d6 | 3H), 7.50-7.45 (m, 1H), 6.95 (d, 2H), 6.66 (s, 1H), 3.76 |
| | | | | | | | (s, 3H) |
| Cpd | LC-3 | 1.26 | 415 | | 400 | DMSO- | 12.10 (s, 1H), 10.70 (s, 1H), 7.71-7.66 (m, 1H), 7.56- |
| 295 | | | | | | d6 | 7.45 (m, 4H), 7.19 (d, 2 H), 6.73 (s, 1 H), 2.29 (s, 3 H). |
| Cpd | LC-2 | 3.35 | 388 | | 400 | DMSO- | 12.03 (s, 1H), 10.46 (s, 1H), 7.81 (d, 1 H), 7.64-7.61 |
| 296 | | | | | | d6 | (m, 2 H), 7.50 (s, 1 H), 7.42 (s, 1 H), 7.32-7.27 (m, 2 |
| | | | | | | | H), 6.54 (s, 1 H), 2.28 (m, 3 H) |
| Cpd | LC-3 | 1.25 | 435 | | 400 | DMSO- | 12.24 (s, 1H), 10.72 (s, 1H), 7.71-7.67 (m, 3H), 7.62 (s, |
| 297 | | | | | | d6 | 1 H), 7.50-7.43 (m, 3 H), 6.86 (s, 1H) |
| Cpd | LC-3 | 1.19 | 431 | | 400 | DMSO- | 11.76 (s, 1H), 10.68 (s, 1H), 7.66 (bs, 1H), 7.59 (d, 1H), |
| 298 | | | | | | d6 | 7.47 (bs, 2H), 7.25 (t, 1H), 7.09 (d, 1 H), 6.98 (t, 1 H), |
| | | | | | | | 6.83 (s, 1 H), 3.85 (s, 3 H) |
| Cpd | LC-2 | 2.88 | 388 | | 400 | DMSO- | 12.23 (s, 1H), 10.52 (s, 1H), 7.80 (d, 1 H), 7.63-7.58 |
| 299 | | | | | | d6 | (m, 2 H), 7.55-7.54 (m, 1 H), 7.11-7.09 (m, 2 H), 6.92 |
| | | | | | | | (s, 1 H), 6.71-6.69 (m, 1 H), 3.80 (s, 3 H). |
| Cpd | LC-2 | 3.02 | 404 | | 400 | DMSO- | 11.97 (s, 1H), 10.47 (s, 1H), 7.81 (d, 1 H), 7.62-7.61 |
| 300 | | | | | | d6 | (m, 2 H), 7.48-7.43 (m, 2 H), 7.12 (d, 1 H), 6.99 (dd, 1 |
| | | | | | | | H), 6.64 (t, 1 H), 3.79 (s, 3 H) |
| Cpd | LC-2 | 3.61 | 437 | | 400 | DMSO- | 12.31 (s, 1H), 10.75 (s, 1H), 7.72-7.70 (m, 2H), 7.56- |
| 301 | | | | | | d6 | 7.51 (m, 1H), 7.50-7.47 (m, 1 H), 7.38-7.31 (m, 1 H), |
| | | | | | | | 7.28-7.23 (m, 1 H), 6.86 (s, 1H). |
| Cpd | LC-2 | 2.82 | 408 | | 300 | DMSO- | 12.34 (s, 1H), 10.76 (s, 1H), 9.16 (d, J = 1.9 Hz, 1H), |
| 302 | | | | | | d6 | 7.92 (d, J = 1.9 Hz, 1H), 7.72 (dd, J = 10.4, 6.7 Hz, 1H), |
| | | | | | | | 7.59 (dd, J = 3.2, 1.8 Hz, 1H), 7.50 (dd, J = 12.3, 6.3 |
| | | | | | | | Hz, 1H), 6.84 (m, 1H). |
| Cpd | LC-2 | 3.27 | 416 | 418 | 300 | DMSO- | 12.37 (s, 1H), 10.77 (s, 1H), 8.39 (d, J = 5.0 Hz, 1H), |
| 303 | | | | | | d6 | 7.78-7.64 (m, 2H), 7.56-7.44 (m, 2H), 7.13-7.03 |
| | | | | | | | (m, 2H), 2.33 (s, 3H). |
| Cpd | LC-2 | 3.19 | 432 | 434 | 300 | DMSO- | 12.26 (s, 1H), 10.74 (s, 1H), 8.25 (d, J = 2.9 Hz, 1H), |
| 304 | | | | | | d6 | 7.81-7.66 (m, 2H), 7.56-7.44 (m, 2H), 7.43 (dd, J = |
| | | | | | | | 8.8, 3.0 Hz, 1H), 6.95 (m, 1H), 3.85 (s, 3H). |
| Cpd | LC-2 | 3.08 | 432 | 434 | 300 | DMSO- | 12.37 (s, 1H), 10.76 (s, 1H), 8.34 (d, J = 5.7 Hz, 1H), |
| 305 | | | | | | d6 | 7.72 (dd, J = 10.4, 6.6 Hz, 1H), 7.57-7.44 (m, 2H), |
| | | | | | | | 7.40 (d, J = 2.4 Hz, 1H), 7.15 (d, J = 2.0 Hz, 1H), 6.84 |
| | | | | | | | (dd, J = 5.8, 2.4 Hz, 1H), 3.87 (s, 3H). |
| Cpd | LC-2 | 3.31 | | 368 | 400 | DMSO- | 11.45 (s, 1H), 10.53 (s, 1H), 7.78 (dd, J = 10.8, 1.8 Hz, |
| 306 | | | | | | d6 | 1H), 7.55 (dd, J = 8.5, 1.8 Hz, 1H), 7.55-7.47 (m, 1H), |
| | | | | | | | 7.39 (dd, J = 3.2, 2.2 Hz, 1H), 7.10 (m, 1H), 6.94 (dd, J = |
| | | | | | | | 15.6, 8.2 Hz, 3H), 6.37 (m, 1H), 3.84 (s, 2H), 2.20 (s, 3H). |
| Cpd | LC-2 | 3.47 | 422 | | 400 | DMSO- | 11.56 (s, 1H), 10.57 (s, 1H), 7.73 (dd, J = 10.8, 1.8 Hz, |
| 307 | | | | | | d6 | 1H), 7.53 (dd, J = 8.5, 1.8 Hz, 1H), 7.50 (d, J = 5.2 Hz, |
| | | | | | | | 1H), 7.50-7.40 (m, 5H), 6.60 (m, 1H), 4.01 (s, 2H). |
| Cpd | LC-2 | 3.64 | 438 | | 400 | DMSO- | 11.54 (s, 1H), 10.57 (s, 1H), 7.74 (dd, J = 10.8, 1.8 Hz, |
| 308 | | | | | | d6 | 1H), 7.55 (dd, J = 8.6, 1.8 Hz, 1H), 7.54-7.45 (m, 1H), |
| | | | | | | | 7.43 (dd, J = 3.2, 2.2 Hz, 1H), 7.35 (m, 1H), 7.19 (dd, J = |
| | | | | | | | 7.8, 1.4 Hz, 1H), 7.16-7.09 (m, 1H), 7.07 (d, J = 2.5 |
| | | | | | | | Hz, 1H), 6.57 (m, 1H), 3.96 (s, 2H). |
| Cpd | LC-2 | 3.2 | 420 | | 300 | DMSO- | 11.51 (s, 1H), 10.53 (s, 1H), 7.74 (dd, J = 10.8, 1.7 Hz, |
| 309 | | | | | | d6 | 1H), 7.57-7.45 (m, 2H), 7.42-7.36 (m, 1H), 7.26 (m, |
| | | | | | | | 1H), 7.19-6.87 (m, 4H), 6.51 (m, 1H), 3.92 (s, 2H). |
| Cpd | LC-2 | 2.66 | 409 | 411 | 300 | DMSO- | 12.45 (s, 1H), 10.58 (s, 1H), 8.86 (dd, J = 4.8, 1.5 Hz, |
| 310 | | | | | | d6 | 1H), 8.26 (dd, J = 8.1, 1.6 Hz, 1H), 7.88-7.78 (m, 1H), |
| | | | | | | | 7.69-7.48 (m, 4H), 6.87 (m, 1H). |
| Cpd | LC-2 | 3.13 | 390 | 392 | 400 | DMSO- | 12.23 (s, 1H), 9.66 (s, 1 H), 8.53 (d, 1 H), 7.79-7.77 (m, |
| 311 | | | | | | d6 | 2 H), 7.29-7.19 (m, 3 H), 7.09-7.04 (m, 1 H), 6.94 (s, |
| | | | | | | | 1H), 3.92 (s, 1 H), 0.76-0.68 (m, 4 H). |
| Cpd | LC-2 | 2.62 | 374 | | 400 | DMSO- | 12.15 (s, 1H), 10.86 (s, 1H), 7.68-7.63 (m, 3H), 7.57- |
| 312 | | | | | | d6 | 7.52 (m, 2H), 7.38 (t, 2H), 7.25 (t, 1H), 6.79 (s, 1H) |
| Cpd | LC-2 | 3.79 | 383 | | 400 | DMSO- | 12.14 (s, 1H), 10.32 (s, 1H), 7.63 (d, 4H), 7.51 (d, 1H), |
| 313 | | | | | | d6 | 7.45 (s, 1H), 7.38 (t, 2H), 7.25 (t, 1H), 6.77 (s, 1H) |
| Cpd | LC-2 | 3.07 | 388 | | 400 | DMSO- | 11.86 (br, s, 1H), 10.51 (s, 1H), 7.80 (d, 1H), 7.60 (br, |
| 314 | | | | | | d6 | s, 2H), 7.50-7.45 (m, 2H), 7.08 (d, 2H), 6.93 (s, 1H), |
| | | | | | | | 3.85 (s, 3H). |
| Cpd | LC-2 | 3.98 | 433 | | 400 | DMSO- | 12.13 (s, 1H), 10.72 (s, 1H), 7.71-7.67 (m, 1H), 7.63 (s, |
| 315 | | | | | | d6 | 1H), 7.55 (d, 1H), 7.49-7.44 (m, 1H), 7.18-7.09 (m, 2H), |
| | | | | | | | 6.77 (ill res d, 1H), 2.29 (s, 3H). |
| Cpd | LC-2 | 3.27 | 392 | | 400 | DMSO- | 12.33 (br, s, 1H), 10.56 (s, 1H), 7.80 (d, 1H), 7.66 (s, |
| 316 | | | | | | d6 | 1H), 7.59-7.55 (m, 4H), 7.28 (d, 1H), 7.04 (s, 1H) |
| Cpd | LC-2 | 2.4 | 358 | | 400 | DMSO- | 12.22 (s, 1H), 10.92 (s, 1H), 7.64 (D, 3H), 7.56 (s, 1H), |
| 317 | | | | | | d6 | 7.46-7.45 (m, 1H), 7.39 (t, 2H), 7.25 (t, 2H), 6.81 (s, 1H) |
| Cpd | LC-2 | 3.35 | 426 | | 400 | DMSO- | 12.36 (s, 1H), 10.77 (s, 1H), 8.16 (s, 1H), 8.00 (d, 1H), |
| 318 | | | | | | d6 | 7.73-7.69 (m, 3H), 7.58 (t, 1H), 7.51-7.46 (m, 1H), 7.03 |
| | | | | | | | (s, 1H) |
| Cpd | LC-2 | 4.03 | 433 | | 400 | DMSO- | 12.13 (s, 1H), 10.73 (s, 1H), 7.72-7.63 (m, 2H), 7.55- |
| 319 | | | | | | d6 | 7.45 (m, 2H), 7.20-7.09 (m, 2H), 6.78 (s, 1H), 2.26 (s, 3H) |
| Cpd | LC-2 | 3.96 | 453 | | 400 | DMSO- | 12.29 (s, 1H), 10.75 (s, 1H), 7.87 (d, 1H), 7.72-7.68 (m, |
| 320 | | | | | | d6 | 2H), 7.50-7.45 (m, 1H), 7.37-7.32 (m, 2H), 6.87 (s, 1H) |
| Cpd | LC-2 | 3.9 | 433 | | 400 | DMSO- | 11.92 (s, 1H), 10.62 (s, 1H), 7.75-7.70 (m, 1H), 7.59 (s, |
| 321 | | | | | | d6 | 1H), 7.48-7.43 (m, 1H), 7.31 (q, 1H), 7.14-7.08 (m, 2H), |
| | | | | | | | 6.43 (s, 1H), 2.13 (s, 3H) |
| Cpd | LC-2 | 1.72 | 359 | 361 | 400 | DMSO- | 12.42 (s, 1H), 10.95 (s, 1H), 8.54 (d, 1H), 7.83-7.78 (m, |
| 322 | | | | | | d6 | 2H), 7.66-7.61 (m, 1H), 7.49-7.41 (m, 2H), 7.27-7.23 |
| | | | | | | | (m, 1H), 7.08 (s, 1H) |
| Cpd | LC-2 | 3.66 | 431 | | 400 | DMSO- | 12.15 (s, 1H), 10.69 (s, 1H), 7.69-7.65 (m, 1H), 7.59 (s, |
| 323 | | | | | | d6 | 1H), 7.49-7.44 (m, 1H), 7.29 (t, 1H), 7.22-7.20 (m, 2H), |
| | | | | | | | 6.83-6.80 (m, 2H), 3.78 (s, 3H) |
| Cpd | LC-2 | 3.76 | 449 | | 400 | DMSO- | 12.03 (s, 1H), 10.69 (s, 1H), 7.71-7.67 (m, 1H), 7.64- |
| 324 | | | | | | d6 | 7.60 (m, 2H), 7.49-7.45 (m, 1H), 6.93 (dd, 1H), 6.85 |
| | | | | | | | (dd, 1H), 6.66 (d, 1H), 3.78 (s, 3H) |
| Cpd | LC-2 | 3.58 | 449 | | 400 | DMSO- | 12.16 (s, 1H), 10.73 (s, 1H), 7.73-7.69 (m, 1H), 7.66 (s, |
| 325 | | | | | | d6 | 1H), 7.51-7.46 (m, 1H), 7.25 (t, 1H), 7.17 (t, 1H), 7.09 |
| | | | | | | | (t, 1H), 6.79 (s, 1H), 3.85 (s, 3H) |
| Cpd | LC-2 | 3.01 | 354 | | 400 | DMSO- | 12.15 (s, 1H), 10.42 (s, 1H), 7.70 (d, 1H), 7.63 (d, 2H), |
| 326 | | | | | | d6 | 7.56 (s, 1H), 7.49 (d, 1H), 7.38 (t, 2H), 7.25 (t, 1H), |
| | | | | | | | 6.78 (s, 1H), 2.41 (s, 3H) |
| Cpd | LC-2 | 3.94 | 453 | | 400 | DMSO- | 12.31 (s, 1H), 10.77 (s, 1H), 7.73-7.68 (m, 3H), 7.52- |
| 327 | | | | | | d6 | 7.48 (m, 2H), 7.28 (t, 1H), 6.86 (s, 1H), |
| Cpd | LC-2 | 3.62 | 401 | | 400 | DMSO- | 12.09 (s, 1H), 9.86 (s, 1H), 7.67 (t, 4H), 7.39 (t, 2H), |
| 328 | | | | | | d6 | 7.30-7.23 (m, 2H), 6.73 (s, 1H). |
| Cpd | LC-2 | 3.39 | 368 | | 400 | DMSO- | 11.83 (s, 1H), 10.42 (s, 1H), 7.80 (d, 1 H), 7.64-7.59 |
| 329 | | | | | | d6 | (m, 2H), 7.43 (s, 1H), 7.22 (d, 1H), 7.08 (s, 1H), 7.04 |
| | | | | | | | (d, 1H), 6.39 (s, 1H), 2.27 (s, 3H), 2.24 (s, 3H) |
| Cpd | LC-2 | 3.22 | 426 | | 400 | DMSO- | 12.47 (s, 1H), 10.77 (s, 1H), 7.85-7.82 (m, 4H), 7.74- |
| 330 | | | | | | d6 | 7.70 (m, 2H), 7.52-7.47 (m, 1H), 7.08 (s, 1H) |
| Cpd | LC-2 | 3.38 | 368 | | 400 | DMSO- | 11.84 (s, 1H), 10.44 (s, 1H), 7.78-7.76 (m, 1H), 7.60- |
| 331 | | | | | | d6 | 7.58 (m, 2H), 7.42(s, 1H), 7.16-7.14 (m, 2H), 7.04 (d, |
| | | | | | | | 1H), 6.41 (s, 1H), 2.27 (s, 3H), 2.23 (s, 3H) |
| Cpd | LC-2 | 3.07 | 402 | 404 | 400 | DMSO- | 12.40 (s, 1H), 10.76 (s, 1H), 8.53 (d, 1H), 7.83-7.77 (m, |
| 332 | | | | | | d6 | 2H), 7.53 (t, 1H), 7.47-7.43 (m, 2H), 7.26-7.23 (m, 1H), |
| | | | | | | | 7.07 (s, 1H). |
| Cpd | LC-2 | 2.48 | 374 | | 400 | DMSO- | 12.24 (s, 1H), 10.87 (s, 1H), 7.98 (d, 1H), 7.66-7.61 (m, |
| 333 | | | | | | d6 | 4H), 7.39 (t, 2H), 7.26 (t, 1H), 6.80 (s, 1H) |
| Cpd | LC-2 | 3.69 | 401 | | 400 | DMSO- | 12.19 (s, 1H), 10.71 (s, 1H), 7.64 (d, 2H), 7.54-7.50 (m, |
| 334 | | | | | | d6 | 2H), 7.45 (t, 1H), 7.39 (t, 2H), 7.25 (t, 1H), 6.80 (s, 1H) |
| Cpd | LC-2 | 3.34 | 418 | 420 | 400 | DMSO- | 12.39 (s, 1H), 10.71 (s, 1H), 8.53 (d, 1H), 7.83-7.77 (m, |
| 335 | | | | | | d6 | 2H), 7.65-7.58 (m, 2H), 7.45 (s, 1H), 7.26-7.23 (m, 1H), |
| | | | | | | | 7.07 (s, 1H). |
| Cpd | LC-2 | 3.58 | 398 | | 400 | DMSO- | 12.31 (s, 1H), 10.28 (s, 1H), 8.53 (d, 1H), 7.82-7.76 (m, |
| 336 | | | | | | d6 | 2H), 7.51-7.46 (m, 2H), 7.41 (br s, 1H), 7.25-7.22 (m, |
| | | | | | | | 1H), 7.04 (s, 1H), 2.36 (s, 3H) |
| Cpd | LC-2 | 3.93 | 415 | | 400 | DMSO- | 12.15 (s, 1H), 10.70 (s, 1H), 7.72-7.68 (m, 1H), 7.58 (s, |
| 337 | | | | | | d6 | 1H), 7.50-7.42(m, 3H), 7.26 (t, 1H), 7.07 (d, 1H), 6.78 |
| | | | | | | | (s, 1H), 2.31 (s, 3H) |
| Cpd | LC-2 | 4.07 | 397 | | 400 | DMSO- | 12.13 (s, 1H), 10.24 (s, 1H), 7.63 (d, 2H), 7.51-7.46 (m, |
| 338 | | | | | | d6 | 3H), 7.38 (t, 2H), 7.25 (t, 1H), 6.78 (s, 1H), 2.36 (s, 3H) |
| Cpd | LC-2 | 2.76 | 408 | 410 | 300 | DMSO | 12.74 (s, 1H), 10.78 (s, 1H), 7.84 (d, J = 3.2 Hz, 1H), |
| 339 | | | | | | d6 | 7.79-7.67 (m, 2H), 7.64 (dd, J = 3.2, 1.7 Hz, 1H), 7.51 |
| | | | | | | | (dd, J = 12.2, 6.3 Hz, 1H), 6.96 (m, 1H). |
| Cpd | LC-2 | 3.44 | 410 | | 400 | DMSO- | 11.46 (s, 1H), 10.50 (s, 1H), 7.76 (dd, J = 10.7, 1.8 Hz, |
| 340 | | | | | | d6 | 1H), 7.60-7.44 (m, 2H), 7.39 (dd, J = 3.2, 2.2 Hz, 1H), |
| | | | | | | | 7.14 (m, 1H), 6.85 (m, 1H), 6.81-6.67 (m, 2H), 6.43 |
| | | | | | | | (m, 1H), 3.86 (s, 2H), 3.72 (m, 1H), 0.79-0.66 (m, |
| | | | | | | | 2H), 0.60 (m, 2H). |
| Cpd | LC-2 | 3.57 | 412 | | 300 | DMSO- | 11.47 (s, 1H), 10.52 (s, 1H), 7.77 (dd, J = 10.8, 1.7 Hz, |
| 341 | | | | | | d6 | 1H), 7.55 (dd, J = 8.6, 1.7 Hz, 1H), 7.56-7.44 (m, 1H), |
| | | | | | | | 7.39 (dd, J = 3.2, 2.2 Hz, 1H), 7.11 (m, 1H), 6.75-6.65 |
| | | | | | | | (m, 2H), 6.65-6.59 (m, 1H), 6.44 (m, 1H), 4.48 (m, |
| | | | | | | | 1H), 3.85 (s, 2H), 1.22 (d, J = 6.0 Hz, 6H). |
| Cpd | LC-2 | 3.63 | 424 | | 400 | DMSO- | 11.47 (s, 1H), 10.50 (s, 1H), 7.75 (dd, J = 10.7, 1.8 Hz, |
| 342 | | | | | | d6 | 1H), 7.55 (dd, J = 8.5, 1.8 Hz, 1H), 7.55-7.46 (m, 1H), |
| | | | | | | | 7.40 (m, 1H), 7.11 (m, 1H), 6.70 (dd, J = 11.7, 8.1 Hz, |
| | | | | | | | 2H), 6.68-6.62 (m, 1H), 6.45 (d, J = 2.4 Hz, 1H), 3.86 |
| | | | | | | | (s, 2H), 3.71 (d, J = 6.9 Hz, 2H), 1.17 (m, 1H), 0.54 (m, |
| | | | | | | | 2H), 0.29 (m, 2H). |
| Cpd | LC-2 | 2.81 | 360 | | 300 | DMSO- | 11.51 (s, 1H), 10.53 (s, 1H), 7.78 (dd, J = 10.8, 1.7 Hz, |
| 343 | | | | | | d6 | 1H), 7.56 (dd, J = 8.5, 1.7 Hz, 1H), 7.57-7.45 (m, 1H), |
| | | | | | | | 7.40 (dd, J = 3.2, 2.2 Hz, 1H), 7.28 (dd, J = 5.1, 1.3 Hz, |
| | | | | | | | 1H), 6.89 (dd, J = 5.1, 3.4 Hz, 1H), 6.83 (dd, J = 3.4, |
| | | | | | | | 1.2 Hz, 1H), 6.55 (m, 1H), 4.11 (s, 2H). |
| Cpd | LC-2 | 3.78 | 470 | | 400 | DMSO- | 12.45 (s, 1H), 10.79 (s, 1H), 8.16-8.06 (m, 2H), 7.79- |
| 344 | | | | | | d6 | 7.69 (m, 2H), 7.66 (dd, J = 3.3, 1.7 Hz, 1H), 7.53 (dd, J = |
| | | | | | | | 12.1, 6.3 Hz, 1H), 7.26 (dd, J = 2.7, 1.6 Hz, 1H). |
| Cpd | LC-2 | 3.63 | 482 | 484 | 400 | DMSO- | 12.52 (s, 1H), 10.80 (s, 1H), 8.66 (d, J = 2.3 Hz, 1H), |
| 345 | | | | | | d6 | 8.07 (dd, J = 8.6, 2.4 Hz, 1H), 7.82 (d, J = 8.6 Hz, 1H), |
| | | | | | | | 7.73 (dd, J = 10.3, 6.6 Hz, 1H), 7.60 (dd, J = 3.2, 1.7 |
| | | | | | | | Hz, 1H), 7.53 (dd, J = 12.2, 6.3 Hz, 1H), 7.20-7.15 (m, 1H). |
| Cpd | LC-2 | 3.56 | 480 | 482 | 400 | DMSO- | 12.43 (s, 1H), 10.80 (s, 1H), 8.62 (dd, J = 4.6, 1.4 Hz, |
| 346 | | | | | | d6 | 1H), 8.18 (dd, J = 8.1, 1.4 Hz, 1H), 7.75 (dd, J = 10.2, |
| | | | | | | | 6.6 Hz, 1H), 7.61 (dd, J = 3.3, 1.6 Hz, 1H), 7.54 (dd, J = |
| | | | | | | | 12.1, 6.3 Hz, 1H), 7.47 (dd, J = 2.7, 1.6 Hz, 1H), 7.26 |
| | | | | | | | (dd, J = 8.1, 4.6 Hz, 1H). |
| Cpd | LC-2 | 2.18 | 359 | 361 | 400 | DMSO- | 12.38 (s, 1H), 10.58 (s, 1H), 8.54 (d, J = 2.8 Hz, 1H), |
| 347 | | | | | | d6 | 7.89 (dd, J = 8.9, 4.3 Hz, 1H), 7.85-7.73 (m, 2H), 7.63 |
| | | | | | | | (dd, J = 3.9, 1.5 Hz, 2H), 7.45 (dd, J = 3.2, 1.7 Hz, 1H), |
| | | | | | | | 7.05 (dd, J = 2.6, 1.7 Hz, 1H). |
| Cpd | LC-2 | 2.38 | 355 | 357 | 400 | DMSO- | 12.23 (s, 1H), 10.51 (s, 1H), 8.45 (dd, J = 4.7, 1.7 Hz, |
| 348 | | | | | | d6 | 1H), 7.86-7.78 (m, 1H), 7.74-7.67 (m, 1H), 7.69- |
| | | | | | | | 7.59 (m, 2H), 7.43 (dd, J = 3.3, 1.6 Hz, 1H), 7.23 (dd, J = |
| | | | | | | | 7.6, 4.7 Hz, 1H), 6.83 (dd, J = 2.6, 1.6 Hz, 1H), 2.43 |
| | | | | | | | (s, 3H). |
| Cpd | LC-2 | 2.5 | 375 | 377 | 400 | DMSO- | 12.42 (s, 1H), 10.58 (s, 1H), 8.58 (dd, J = 4.5, 1.4 Hz, |
| 349 | | | | | | d6 | 1H), 8.01 (dd, J = 8.1, 1.4 Hz, 1H), 7.88-7.80 (m, 1H), |
| | | | | | | | 7.69-7.59 (m, 2H), 7.50 (dd, J = 3.3, 1.6 Hz, 1H), 7.39- |
| | | | | | | | 7.31 (m, 2H). |
| Cpd | LC-2 | 2.58 | 420 | 421 | 400 | DMSO- | 12.38 (s, 1H), 10.60 (s, 1H), 8.61 (dd, J = 4.6, 1.4 Hz, |
| 350 | | | | | | d6 | 1H), 8.17 (dd, J = 8.1, 1.4 Hz, 1H), 7.87-7.80 (m, 1H), |
| | | | | | | | 7.64 (dd, J = 3.8, 1.9 Hz, 2H), 7.47 (m, 2H), 7.25 (dd, J = |
| | | | | | | | 8.1, 4.6 Hz, 1H). |
| Cpd | LC-2 | 3.86 | 435 | | 400 | DMSO- | 11.87 (s, 1H), 10.69 (s, 1H), 7.56-7.55 (m, 1H), 7.54 |
| 351 | | | | | | d6 | (m, 1H), 7.44 (bs, 2H), 7.40-7.29 (m, 3H), 6.75 (s, 1H) |
| Cpd | LC-2 | 3.07 | 388 | | 400 | DMSO- | 11.77 (s, 1H), 10.48 (s, 1H), 7.78 (d, 1 H), 7.63-7.56 |
| 352 | | | | | | d6 | (m, 3H), 7.40 (s, 1 H), 7.01 (dd, 1 H), 6.85-6.80 (m, |
| | | | | | | | 1H), 6.76 (s, 1H), 3.86 (s, 3H); |
| Cpd | LC-2 | 1.67 | 359 | 361 | 400 | DMSO- | 12.44 (s, 1H), 10.93 (s, 1H), 8.53 (d, 1H), 7.94-7.90 (m, |
| 353 | | | | | | d6 | 1H), 7.83-7.80 (m, 2H), 7.58 (s, 1H), 7.51-7.47 (m, 1H), |
| | | | | | | | 7.26 (q, 1H), 7.09 (s, 1H) |
| Cpd | LC-2 | 1.62 | 359 | 361 | 400 | DMSO- | 12.28 (s, 1H), 10.01 (s, 1H), 8.54 (d, 1H), 7.85-7.79 (m, |
| 354 | | | | | | d6 | 4H), 7.26-7.23 (m, 2H), 6.98 (s, 1H) |
| Cpd | LC-2 | 3.29 | 426 | | 400 | DMSO- | 12.43 (s, 1H), 10.81 (s, 1H), 7.89 (d, 1H), 7.78-7.69 (m, |
| 355 | | | | | | d6 | 4H), 7.52-7.46 (m, 2H), 7.06 (s, 1H) |
| Cpd | LC-2 | 3.94 | 435 | | 400 | DMSO- | 12.30 (s, 1H), 10.74 (s, 1H), 7.77 (s, 1H), 7.30-7.68 (m, |
| 356 | | | | | | d6 | 1H), 7.65-7.62 (m, 2H), 7.51-7.46 (m, 1H), 7.42-7.38 |
| | | | | | | | (m, 1H), 7.31-7.29 (m, 1H), 6.94(s, 1H); |
| Cpd | LC-2 | 1.88 | 375 | 377 | 400 | DMSO- | 12.43 (s, 1H), 10.91 (s, 1H), 8.53 (d, 1H), 7.99 (d, 1H), |
| 357 | | | | | | d6 | 7.79 (s, 2H), 7.64 (d, 1H), 7.51 (s, 1H), 7.26-7.25 (m, |
| | | | | | | | 1H), 7.07 (s, 1H) |
| Cpd | LC-2 | 3.24 | 344 | | 400 | DMSO- | 11.65 (s, 1H), 10.41 (s, 1H), 7.79 (d, 1 H), 7.59-7.54 |
| 358 | | | | | | d6 | (m, 2 H), 7.33 (s, 1 H), 6.24 (s, 1 H), 6.11 (s, 1H), 2.18 |
| | | | | | | | (m, 2H), 2.09 (m, 2H), 1.65-1.62 (m, 2H), 1.55-1.54 (m, 2 H); |
| Cpd | LC-2 | 3.69 | | 431 | 400 | DMSO- | 11.83 (br s, 1H), 10.12 (s, 1H), 7.55-7.51 (m, 1H), 7.46 |
| 359 | | | | | | d6 | (s, 1H), 7.31-7.27 (m, 3H), 7.12-7.00 (m, 3H); |
| Cpd | LC-2 | 3.5 | 417 | | 400 | DMSO- | 11.78 (br s, 1H), 9.99 (s, 1H), 7.41 (br s, 1H), 7.35-7.19 |
| 360 | | | | | | d6 | (m, 4H), 7.16-6.96 (m, 4H); |
| Cpd | LC-2 | 3.95 | 469 | | 400 | DMSO- | 12.10 (s, 1H), 10.64(s, 1H), 7.80 (d, 1H), 7.73-7.68 (m, |
| 361 | | | | | | d6 | 2H), 7.61-7.57 (m, 2H), 7.53 (d, 1H), 7.44-7.40 (m, 1H), |
| | | | | | | | 6.42 (s, 1H) |
| Cpd | LC-2 | 4.04 | 453 | | 400 | DMSO- | 12.26 (s, 1H), 10.74 (s, 1H), 7.78-7.71 (m, 3H), 7.55- |
| 362 | | | | | | d6 | 7.47 (m, 2H), 7.36 (d, 1H), 6.82 (s, 1H) |
| Cpd | LC-2 | 3.8 | 453 | | 400 | DMSO- | 12.08 (s, 1H), 10.66 (s, 1H), 7.75-7.70 (m, 1H), 7.65 (s, |
| 363 | | | | | | d6 | 1H), 7.47-7.44 (m, 3H), 7.35-7.31(m, 1H), 6.57 (s, 1H) |
| Cpd | LC-2 | 3.66 | 419 | | 400 | DMSO- | 11.90 (br s, 1H), 10.60 (s, 1H), 7.58-7.55 (m, 2H), 7.33- |
| 364 | | | | | | d6 | 7.18 (m, 4H), 7.11 (br s, 1H), 7.03-6.99 (m, 1H) |
| Cpd | LC-2 | 4.09 | 469 | | 400 | DMSO- | 12.43 (s, 1H), 10.74 (s, 1H), 8.05 (s, 1H), 7.97 (d, 1H), |
| 365 | | | | | | d6 | 7.73-7.70 (m, 2H), 7.63-7.60 (m, 2H), 7.52-7.47 (m, |
| | | | | | | | 1H), 7.03 (s, 1H) |
| Cpd | LC-2 | 3.71 | 437 | | 400 | DMSO- | 12.2 (s, 1H), 10.74 (s, 1H), 7.79-7.68 (m, 3H), 7.55- |
| 366 | | | | | | d6 | 7.46 (m, 1H), 7.36 (t, 1H), 7.18 (t, 1H), 6.76 (s, 1H) |
| Cpd | LC-2 | 3.21 | 400 | | 400 | DMSO- | 11.48 (s, 1H), 10.54 (s, 1H), 7.76 (dd, J = 10.8, 1.8 Hz, |
| 367 | | | | | | d6 | 1H), 7.55 (dd, J = 8.5, 1.8 Hz, 1H), 7.50 (m, 1H), 7.39 |
| | | | | | | | (m, 1H), 7.16 (m, 1H), 7.07-6.98 (m, 2H), 6.92 (d, J = |
| | | | | | | | 7.6 Hz, 1H), 6.46 (d, J = 2.4 Hz, 1H), 3.87 (s, 2H), 2.40 |
| | | | | | | | (s, 3H). |
| Cpd | LC-2 | 2.78 | 398 | | 400 | DMSO- | 11.45 (s, 1H), 10.53 (s, 1H), 7.77 (dd, J = 10.8, 1.8 Hz, |
| 368 | | | | | | d6 | 1H), 7.55 (dd, J = 8.5, 1.8 Hz, 1H), 7.50 (m, 1H), 7.39 |
| | | | | | | | (m, 1H), 7.20 (m, 1H), 7.13-7.04 (m, 3H), 6.38 (d, J = |
| | | | | | | | 2.5 Hz, 1H), 4.31 (s, 2H), 3.88 (s, 2H), 3.25 (s, 3H). |
| Cpd | LC-2 | 3.63 | 470 | | 400 | DMSO- | 12.69 (s, 1H), 10.82 (s, 1H), 8.90 (d, J = 2.3 Hz, 1H), |
| 369 | | | | | | d6 | 8.21 (dd, J = 8.5, 2.4 Hz, 1H), 8.05 (d, J = 8.4 Hz, 1H), |
| | | | | | | | 7.74 (dd, J = 10.3, 6.5 Hz, 1H), 7.66 (dd, J = 3.2, 1.6 |
| | | | | | | | Hz, 1H), 7.53 (dd, J = 12.2, 6.3 Hz, 1H), 7.32 (d, J = 2.1 |
| | | | | | | | Hz, 1H). |
| Cpd | LC-2 | 3.64 | 470 | | 400 | DMSO- | 12.63 (s, 1H), 10.82 (s, 1H), 8.80 (d, J = 5.1 Hz, 1H), |
| 370 | | | | | | d6 | 8.25 (s, 1H), 7.74 (dd, J = 10.3, 6.6 Hz, 1H), 7.65 (dd, J = |
| | | | | | | | 3.2, 1.7 Hz, 1H), 7.60 (d, J = 5.2 Hz, 1H), 7.52 (dd, J = |
| | | | | | | | 12.2, 6.3 Hz, 1H), 7.39 (m, 1H). |
| Cpd | LC-2 | 3.51 | 480 | | 300 | DMSO- | 12.43 (s, 1H), 10.78 (s, 1H), 7.85 (d, J = 7.7 Hz, 1H), |
| 371 | | | | | | d6 | 7.81-7.68 (m, 2H), 7.60 (dd, J = 3.3, 1.7 Hz, 1H), 7.50 |
| | | | | | | | (dd, J = 10.8, 7.0 Hz, 2H), 7.15 (m, 1H). |
| Cpd | LC-2 | 2.48 | 375 | | 400 | DMSO- | 12.42 (s, 1H), 10.59 (s, 1H), 7.91-7.77 (m, 3H), 7.61 |
| 372 | | | | | | d6 | (d, J = 5.2 Hz, 2H), 7.49 (dd, J = 3.2, 1.7 Hz, 1H), 7.35 |
| | | | | | | | (d, J = 7.7 Hz, 1H), 7.12 (m, 1H). |
| Cpd | LC-2 | 2.88 | 409 | | 300 | DMSO- | 12.40 (s, 1H), 10.59 (s, 1H), 8.14-8.03 (m, 2H), 7.88- |
| 373 | | | | | | d6 | 7.78 (m, 1H), 7.73 (dd, J = 4.9, 3.6 Hz, 1H), 7.62 (dd, J = |
| | | | | | | | 3.7, 1.6 Hz, 2H), 7.54 (dd, J = 3.3, 1.7 Hz, 1H), 7.21 |
| | | | | | | | (m, 1H). |
| Cpd | LC-2 | 2.61 | 419 | | 400 | DMSO- | 12.39 (s, 1H), 10.58 (s, 1H), 7.83 (d, J = 10.6 Hz, 2H), |
| 374 | | | | | | d6 | 7.75 (m, 1H), 7.61 (d, J = 5.8 Hz, 2H), 7.52-7.45 (m, |
| | | | | | | | 2H), 7.11 (m, 1H). |
| Cpd | LC-4 | 1.35 | 372 | 374 | 300 | DMSO- | 12.26 (s, 1H), 11.34 (s, 1H), 7.72-7.62 (m, 2H), 7.61- |
| 375 | | | | | | d6 | 7.48 (m, 2H), 7.40 (m, 2H), 7.27 (m, 1H), 6.78 (m, 1H) |
| Cpd | LC-5 | 3.16 | 417 | | 400 | DMSO- | 12.10 (s, 1H), 10.70 (s, 1H), 7.69-7.60 (m, 4H), 7.48 (s, |
| 378 | | | | | | d6 | 1H), 7.38 (t, 2H), 7.24 (t, 1H), 6.76 (s, 1H) |
| Cpd | LC-5 | 2.97 | | 420 | 400 | DMSO- | 12.40 (s, 1H), 10.74 (s, 1H), 8.54 (d, 1H), 7.79-7.77 (m, |
| 379 | | | | | | d6 | 3H), 7.65 (d, 1H), 7.46 (s, 1H), 7.25-7.24 (m, 1H), 7.06 |
| | | | | | | | (s, 1H) |
| Cpd | LC-5 | 2.88 | | 393 | 400 | DMSO- | 12.80 (s, 1H), 10.54 (s, 1H), 8.98 (d, 1H), 8.45 (d, 1H), |
| 380 | | | | | | d6 | 8.14 (d, 1H), 7.90 (d, 1H), 7.78 (d, 1H), 7.67-7.59 (m, |
| | | | | | | | 4H), 7.53 (s, 1H), 7.25 (s, 1H) |
| Cpd | LC-5 | 2.90 | 390 | | 400 | DMSO- | 11.92 (s, 1H), 10.04(s, 1H), 7.88-7.83 (m, 2H), 7.63 (s, |
| 381 | | | | | | d6 | 1H), 7.48-7.41 (m, 4H), 7.28-7.23 (m, 4H), 6.92 (s, 1H) |
| Cpd | LC-5 | 2.94 | 390 | | 400 | DMSO- | 12.20 (s, 1H), 10.52 (s, 1H), 8.00-7.98 (m, 1H), 7.95- |
| 382 | | | | | | d6 | 7.93 (m, 2H), 7.88-7.85 (m, 1H), 7.70-7.64 (m, 2H), |
| | | | | | | | 7.58-7.50 (m, 5H), 6.58 (s, 1H) |
| Cpd | LC-5 | 2.96 | | 400 | 400 | DMSO- | 12.35 (s, 1H), 10.35 (s, 1H), 8.53 (d, 1H), 7.82-7.76 (m, |
| 383 | | | | | | d6 | 2H), 7.49-7.43 (m, 2H), 7.40 (m, 1H), 7.26-7.23 (m, |
| | | | | | | | 1H), 7.05 (s, 1H), 2.25 (s, 3H) |
| Cpd | LC-12 | 1.87 | 368 | | 400 | DMSO- | 11.40 (s, 1H), 10.64 (s, 1H), 7.82-7.75 (m, 1H), 7.62- |
| 384 | | | | | | d6 | 7.53 (m, 2H), 7.43 (m, 1H), 7.11 (m, 2H), 7.05 (m, 1H), |
| | | | | | | | 6.98 (d, J = 6.7 Hz, 1H), 6.00 (d, J = 2.3 Hz, 1H), 3.84 |
| | | | | | | | (s, 2H), 2.07 (s, 3H) |
| Cpd | LC-12 | 1.92 | 368 | | 300 | DMSO- | 11.44 (s, 1H), 10.49 (s, 1H), 7.78 (dd, J = 10.8, 1.7 Hz, |
| 385 | | | | | | d6 | 1H), 7.60-7.45 (m, 2H), 7.37 (dd, J = 3.2, 2.2 Hz, 1H), |
| | | | | | | | 7.02 (s, 4H), 6.35 (d, J = 2.3 Hz, 1H), 3.83(s, 2H), 2.25 |
| | | | | | | | (s, 3H). |
| Cpd | LC-13 | 1.34 | 422 | | 300 | DMSO- | 11.49 (s, 1H), 10.57 (s, 1H), 7.85-7.75 (m, 1H), 7.69 |
| 386 | | | | | | d6 | (dd, J = 7.8, 1.5 Hz, 1H), 7.61-7.55 (m, 2H), 7.50 (dd, |
| | | | | | | | J = 7.5, 1.6 Hz, 1H), 7.45-7.40 (m, 2H), 7.17 (d, J = |
| | | | | | | | 7.6 Hz, 1H), 6.11 (m, 1H), 4.05 (s, 2H) |
| Cpd | LC-12 | 1.99 | 422 | | 300 | DMSO- | 11.49 (s, 1H), 10.57 (s, 1H), 7.85-7.75 (m, 1H), 7.69 |
| 387 | | | | | | d6 | (dd, J = 7.8, 1.5 Hz, 1H), 7.61-7.55 (m, 2H), 7.50 (dd, |
| | | | | | | | J = 7.5, 1.6 Hz, 1H), 7.45-7.40 (m, 2H), 7.17 (d, J = |
| | | | | | | | 7.6 Hz, 1H), 6.11 (m, 1H), 4.05 (s, 2H) |
| Cpd | LC-13 | 1.37 | 438 | | 400 | DMSO- | 11.50 (s, 1H), 10.58 (s, 1H), 7.78 (dd, J = 10.6, 1.6 Hz, |
| 388 | | | | | | d6 | 1H), 7.61-7.50 (m, 2H), 7.44 (m, 1H), 7.34 (m, 2H), |
| | | | | | | | 7.24 (m, 1H), 7.18-7.12 (m, 1H), 6.25 (d, J = 2.4 Hz, |
| | | | | | | | 1H), 3.94 (s, 2H) |
| Cpd | LC-14 | 1.87 | 438 | | 300 | DMSO- | 11.53 (s, 1H), 10.54 (s, 1H), 7.74 (dd, J = 10.8, 1.8 Hz, |
| 389 | | | | | | d6 | 1H), 7.58-7.45 (m, 2H), 7.41 (dd, J = 3.2, 2.2 Hz, 1H), |
| | | | | | | | 7.31-7.22 (m, 2H), 7.19 (d, J = 8.4 Hz, 2H), 6.53 (m, |
| | | | | | | | 1H), 3.94 (s, 2H) |
| Cpd | LC-15 | 1.25 | | 399 | 400 | DMSO- | 11.41 (s, 1H), 10.48 (s, 1H), 7.76 (s, 1H), 7.53 (s, 2H), |
| 390 | | | | | | d6 | 7.37 (s, 1H), 7.03 (s, 1H), 6.69-6.14 (m, 4H), 3.80 (s, |
| | | | | | | | 2H), 2.81 (s, 6H) |
| Cpd | LC-13 | 1.34 | 432 | | 400 | DMSO- | 11.55 (s, 1H), 10.54 (s, 1H), 7.75 (dd, J = 10.8, 1.8 Hz, |
| 391 | | | | | | d6 | 1H), 7.54 (dd, J = 8.6, 1.8 Hz, 1H), 7.49 (m, 1H), 7.42 |
| | | | | | | | (m, 1H), 7.33 (m, 1H), 7.25 (d, J = 2.0 Hz, 1H), 7.22- |
| | | | | | | | 7.13 (m, 2H), 6.56 (d, J = 2.4 Hz, 1H), 3.91 (s, 2H) |
| Cpd | LC-16 | 1.63 | 360 | | 300 | DMSO- | 11.46 (s, 1H), 10.55 (s, 1H), 7.83-7.73 (m, 1H), 7.63 |
| 392 | | | | | | d6 | 7.45 (m, 2H), 7.39 (m, 2H), 7.11 (d, J = 3.0 Hz, 1H), |
| | | | | | | | 6.93 (d, J = 4.9 Hz, 1H), 6.46 (d, J = 2.4 Hz, 1H), 3.90 |
| | | | | | | | (s, 2H) |
| Cpd | LC-5 | 3.12 | 437 | | 400 | DMSO- | 12.28 (s, 1H), 10.77 (s, 1H), 7.75-7.71 (m, 2H), 7.66- |
| 393 | | | | | | d6 | 7.62 (m, 1H), 7.52-7.47 (m, 1H), 7.38-7.32 (m, 1H), |
| | | | | | | | 7.19-7.14 (m, 1H), 6.89 (s, 1H) |
| Cpd | LC-5 | 2.96 | 404 | | 400 | DMSO- | 12.03 (s, 1H), 10.53 (s, 1H), 7.81 (d, 1H), 7.63-7.56 (m, |
| 394 | | | | | | d6 | 3H), 7.49 (br s, 1H), 7.41 (d, 1H), 7.19 (t, 1H), 6.89 (br |
| | | | | | | | s, 1H), 3.58 (s, 3H) |
| Cpd | LC-5 | 3.14 | 396 | | 400 | DMSO- | 11.76 (s, 1H), 10.21 (s, 1H), 7.67 (d, 1H), 7.45-7.41 (m, |
| 395 | | | | | | d6 | 3H), 7.32 (t, 1H), 7.27-7.23 (m, 2H), 7.20 (t, 1H), 7.02- |
| | | | | | | | 7.00 (m, 1H), 1.26 (s, 9H) |
| Cpd | LC-5 | 2.70 | | 393 | 400 | DMSO- | 12.47 (s, 1H), 10.52 (s, 1H), 8.52 (d, 1H), 8.37 (d, 1H), |
| 396 | | | | | | d6 | 8.04 (d, 1H), 7.85-7.72 (m, 4H), 7.69-7.60 (m, 2H), 7.54 |
| | | | | | | | (br s, 1H), 7.04 (br s, 1H) |
| Cpd | LC-5 | 3.05 | 396 | | 400 | DMSO- | 11.71 (s, 1H), 10.53 (s, 1H), 7.80 (d, 1H), 7.65-7.55 (m, |
| 397 | | | | | | d6 | 3H), 7.43-7.37 (m, 2H), 7.27 (t, 1H), 7.00 (t, 1H), 6.75 |
| | | | | | | | (br s, 1H), 3.93-3.88 (m, 1H), 0.84-0.79 (m, 2H), 0.71- |
| | | | | | | | 0.67 (m, 2H) |
| Cpd | LC-5 | 2.74 | 346 | | 400 | DMSO- | 11.83 (s, 1H), 10.45 (s, 1H), 7.71 (d, 1H), 7.52-7.49 (m, |
| 398 | | | | | | d6 | 2H), 7.42 (t, 1H), 7.37 (d, 1H), 7.32-7.30 (m, 1H), 7.08- |
| | | | | | | | 7.07 (m, 1H), 7.01-6.99 (m, 1H) |
| Cpd | LC-5 | 3.06 | 415 | | 400 | DMSO- | 12.12 (s, 1H), 10.10 (s, 1H), 7.63-7.60 (m, 3H), 7.38 (t, |
| 399 | | | | | | d6 | 2H), 7.36-7.34 (m, 2H), 7.25 (t, 1H), 7.16 (t, 1H), 6.71 |
| | | | | | | | (br s, 1H) |
| Cpd | LC-5 | 2.75 | 346 | | 400 | DMSO- | 11.78 (s, 1H), 10.51 (s, 1H), 7.74-7.67 (m, 2H), 7.53- |
| 400 | | | | | | d6 | 7.41 (m, 4H), 7.33 (d, 1H), 7.19-7.18 (m, 1H); |
| Cpd | LC-5 | 2.87 | | 362 | 400 | DMSO- | 11.70 (s, 1H), 10.46 (s, 1H), 7.70 (d, 1H), 7.49 (d, 1H), |
| 401 | | | | | | d6 | 7.44-7.39 (m, 3H), 7.11-7.09 (m, 1H), 6.98 (s, 1H), 2.40 |
| | | | | | | | (s, 3H) |
| Cpd | LC-5 | 2.98 | 380 | | 400 | DMSO- | 11.74 (s, 1H), 10.31 (s, 1H), 7.67 (d, 1H), 7.46-7.44 (m, |
| 402 | | | | | | d6 | 2H), 7.35 (t, 1H), 7.24-7.22 (m, 1H), 7.14 (t, 1H), 7.09 |
| | | | | | | | (br s, 1H), 7.00-6.99 (m, 1H), 6.92 (d, 1H), 1.87-1.83 |
| | | | | | | | (m, 1H), 0.94-0.89 (m, 2H), 0.66-0.62 (m, 2H) |
| Cpd | LC-13 | 1.18 | | 408 | 300 | DMSO- | 12.64 (s, 1H), 10.15 (s, 1H), 7.84 (d, J = 3.3 Hz, 1H), |
| 403 | | | | | | d6 | 7.69 (d, J = 3.3 Hz, 1H), 7.47-7.34 (m, 3H), 7.34- |
| | | | | | | | 6.90 (m, 1H), 6.86 (m, 1H) |
| Cpd | LC-13 | 1.27 | | 420 | 300 | DMSO- | 12.42 (s, 1H), 10.17 (s, 1H), 8.44 (dd, J = 4.0, 2.1 Hz, |
| 404 | | | | | | d6 | 1H), 7.81 (m, 1H), 7.49-6.95 (m, 5H), 6.93 (m, 1H) |
| Cpd | LC-13 | 1.24 | | 420 | 300 | DMSO- | 12.66 (s, 1H), 10.29 (s, 1H), 7.83 (d, J = 3.2 Hz, 1H), |
| 405 | | | | | | d6 | 7.77-7.66 (m, 2H), 7.45 (dd, J = 3.2, 1.7 Hz, 1H), 7.35 |
| | | | | | | | (dd, J = 9.9, 6.8 Hz, 1H), 6.88 (m, 1H) |
| Cpd | LC-13 | 1.35 | 430 | 432 | 300 | DMSO- | 12.44 (s, 1H), 10.29 (s, 1H), 8.44 (m, 1H), 7.81 (m, 1H), |
| 406 | | | | | | d6 | 7.71 (dd, J = 9.7, 6.4 Hz, 1H), 7.56-7.20 (m, 3H), 6.94 |
| | | | | | | | (m, 1H) |
| Cpd | LC-12 | 1.84 | 372 | | 300 | DMSO- | 11.55 (s, 1H), 10.94 (s, 1H), 7.87 (dd, J = 10.3, 6.0 Hz, |
| 407 | | | | | | d6 | 1H), 7.56 (m, 1H), 7.34 (dd, J = 11.2, 6.4 Hz, 1H), 7.28- |
| | | | | | | | 7.05 (m, 5H), 6.45 (d, J = 2.4 Hz, 1H), 3.91 (s, 2H) |
| Cpd | LC-13 | 1.19 | | 384 | 300 | DMSO- | 12.09 (s, 1H), 10.67 (s, 1H), 8.13 (d, J = 2.4 Hz, 1H), |
| 408 | | | | | | d6 | 7.79 (dd, J = 10.6, 2.5 Hz, 1H), 7.73-6.94 (m, 7H), |
| | | | | | | | 6.88 (m, 1H) |
| Cpd | LC-17 | 1.84 | 417 | | 300 | DMSO- | 12.13 (s, 1H), 10.12 (s, 1H), 7.74 (m, 1H), 7.52-6.90 |
| 409 | | | | | | d6 | (m, 7H), 6.76 (m, 1H) |
| Cpd | LC-17 | 1.96 | 451 | | 300 | DMSO- | 12.24 (s, 1H), 10.14 (s, 1H), 7.92-7.83 (m, 1H), 7.53 |
| 410 | | | | | | d6 | (dd, J = 3.2, 1.6 Hz, 1H), 7.48-6.92 (m, 5H), 6.83 (m, 1H) |
| Cpd | LC-17 | 1.84 | 417 | | 300 | DMSO- | 12.13 (s, 1H), 10.08 (s, 1H), 7.76-7.61 (m, 2H), 7.48- |
| 411 | | | | | | d6 | 7.15 (m, 6H), 6.72 (dd, J = 2.6, 1.7 Hz, 1H) |
| Cpd | LC-17 | 1.89 | 435 | | 300 | DMSO- | 12.13 (s, 1H), 10.08 (s, 1H), 7.76-7.61 (m, 2H), 7.48- |
| 412 | | | | | | d6 | 7.15 (m, 6H), 6.72 (dd, J = 2.6, 1.7 Hz, 1H) |
| Cpd | LC-17 | 1.82 | 429 | | 300 | DMSO- | 12.14 (s, 1H), 10.25 (s, 1H), 7.80-7.65 (m, 2H), 7.52 |
| 413 | | | | | | d6 | (dd, J = 3.2, 1.7 Hz, 1H), 7.41-7.20 (m, 4H), 6.76 (m, 1H) |
| Cpc | LC-17 | 1.95 | 463 | | 300 | DMSO- | 12.26 (s, 1H), 10.27 (s, 1H), 7.92-7.83 (m, 1H), 7.71 |
| 414 | | | | | | d6 | (dd, J = 9.7, 6.4 Hz, 1H), 7.58 (dd, J = 3.2, 1.7 Hz, 1H), |
| | | | | | | | 7.42-7.29 (m, 3H), 6.84 (m, 1H) |
| Cpd | LC-17 | 1.59 | 402 | | 300 | DMSO- | 11.31 (s, 1H), 9.76 (s, 1H), 7.36-7.23 (m, 3H), 7.22- |
| 415 | | | | | | d6 | 7.12 (m, 5H), 6.31-6.24 (m, 1H), 5.23 (s, 2H), 3.83 (s, 2H) |
| Cpd | LC-17 | 1.59 | 378 | | 300 | DMSO- | 11.60 (s, 1H), 10.94 (s, 1H), 7.87 (dd, J = 10.3, 6.0 Hz, |
| 416 | | | | | | d6 | 1H), 7.57 (dd, J = 3.2, 2.2 Hz, 1H), 7.33 (dd, J = 11.2, |
| | | | | | | | 6.4 Hz, 1H), 7.26 (dd, J = 5.1, 1.3 Hz, 1H), 6.87 (dd, J = |
| | | | | | | | 5.1, 3.4 Hz, 1H), 6.81 (dd, J = 3.4, 1.2 Hz, 1H), 6.62 (m, |
| | | | | | | | 1H), 4.12 (s, 2H) |
| Cpd | LC-17 | 1.80 | 431 | | 300 | DMSO- | 11.45 (s, 1H), 10.21 (s, 1H), 7.67 (dd, J = 9.7, 6.4 Hz, |
| 417 | | | | | | d6 | 1H), 7.38-7.16 (m, 3H), 6.91 (dd, J = 5.1, 3.4 Hz, 1H), |
| | | | | | | | 6.84 (m, 1H), 6.52 (dd, J = 3.0, 1.8 Hz, 1H), 4.08 (s, 2H) |
| Cpd | LC-17 | 1.86 | 421 | | 300 | DMSO- | 11.56 (s, 1H), 10.75 (s, 1H), 7.67 (dd, J = 10.3, 6.6 Hz, |
| 418 | | | | | | d6 | 1H), 7.51 (dd, J = 3.2, 2.2 Hz, 1H), 7.36 (dd, J = 12.3, |
| | | | | | | | 6.3 Hz, 1H), 7.26 (dd, J = 5.1, 1.3 Hz, 1H), 6.88 (dd, J = |
| | | | | | | | 5.1, 3.4 Hz, 1H), 6.83 (dd, J = 3.4, 1.2 Hz, 1H), 6.59 (m, |
| | | | | | | | 1H), 4.12 (s, 2H) |
| Cpd | LC-5 | 2.88 | | 418 | 400 | DMSO- | 12.30 (s, 1H), 10.15 (s, 1H), 8.52 (d, 1H), 7.82-7.74 (m, |
| 419 | | | | | | d6 | 2H), 7.62 (d, 1H), 7.36-7.16 (m, 4H), 6.98-6.97 (m, 1H) |
| Cpd | LC-5 | 2.84 | 360 | | 400 | DMSO- | 11.77 (s, 1H), 10.39 (s, 1H), 7.72 (d, 1H), 7.51-7.40 (m, |
| 420 | | | | | | d6 | 3H), 7.07-7.06 (m, 1H), 7.00-6.98 (m, 1H), 6.66-6.65 |
| | | | | | | | (m, 1H), 2.39 (s, 3H) |
| Cp | LC-5 | 3.02 | 346 | | 400 | DMSO- | 11.43 (s, 1H), 10.35 (s, 1H), 7.79 (d, 1H), 7.58-7.54 (m, |
| 421 | | | | | | d6 | 2H), 7.26 (s, 1H), 6.00 (s, 1H), 1.86-1.83 (m, 2H), 1.70- |
| | | | | | | | 1.55 (m, 3H), 1.32-1.14 (m, 6H) |
| Cp | LC-6 | 6.12 | | 382 | 400 | DMSO- | 12.11 (s, 1H), 9.78 (s, 1H), 7.73 (d, 1H), 7.63 (d, 2H), |
| 422 | | | | | | d6 | 7.50 (s, 1H), 7.38 (t, 2H), 7.25 (t, 1H), 6.78 (s, 1H), 3.77 |
| | | | | | | | (s, 3H) |
| Cp | LC-5 | 3.29 | 407 | | 400 | DMSO- | 11.44 (s, 1H), 10.54 (s, 1H), 7.69-7.65 (m, 1H), 7.42- |
| 423 | | | | | | d6 | 7.37 (m, 1H), 7.34 (s, 1H), 6.02 (s, 1H), 1.86-1.83 (m, |
| | | | | | | | 2H), 1.71-1.62 (m, 3H), 1.33-1.14 (m, 6H) |
| Cpd | LC-7 | 2.36 | 395 | | 400 | DMSO- | 11.60 (br s, 1H), 10.58 (s, 1H), 7.69-7.67 (m, 1H), 7.43- |
| 424 | | | | | | d6 | 7.39 (m, 2H), 6.15 (s, 1H), 3.91 (t, 1H), 3.82-3.78 (m, |
| | | | | | | | 1H), 3.75-3.71 (m, 1H), 3.47 (t, 1H), 3.35-3.31 (m, 1H), |
| | | | | | | | 2.19-2.16 (m, 1H), 1.88-1.83 (m, 1H) |
| Cpd | LC-5 | 3.03 | 433 | | 400 | DMSO- | 11.81 (s, 1H), 10.06 (s, 1H), 7.46 (d, 1H), 7.39 (bs, 1H), |
| 425 | | | | | | d6 | 7.30-6.89 (m, 7H) |
| Cp | LC-5 | 3.15 | 429 | | 400 | DMSO- | 11.35 (s, 1H), 10.11 (s, 1H), 7.58 (d, 1H), 7.31-7.29 (m, |
| 426 | | | | | | d6 | 1H), 7.26-6.94 (m, 7H), 6.29 (s, 1H), 3.85 (s, 2H) |
| Cpc | LC-5 | 3.08 | | 396 | 400 | DMSO- | 11.39 (s, 1H), 9.64 (s, 1H), 7.59 (d, 1H), 7.35 (s, 1H), |
| 427 | | | | | | d6 | 7.22-7.14 (m, 5H), 6.38 (s, 1H), 3.88 (s, 2H), 3.76 (s, 3H) |
| Cp | LC-5 | 3.11 | | 400 | 400 | DMSO- | 11.80 (s, 1H), 9.59 (s, 1H), 7.47 (m, 2H), 7.32-7.23 (m, |
| 428 | | | | | | d6 | 3H), 7.08-7.00 (m, 2H), 3.62 (m, 2H) |
| Cp | LC-5 | 2.78 | | 396 | 400 | DMSO- | 12.00 (s, 1H), 9.72 (s, 1H), 7.76 (s, 1H), 7.63 (d, 2H), |
| 429 | | | | | | d6 | 7.48 (s, 1H), 7.40-7.00 (m, 5H), 6.87 (s, 1H), 3.81 (s, 3H) |
| Cpd | LC-5 | 3.10 | 379 | | 400 | DMSO- | 11.54 (s, 1H), 10.56 (s, 1H), 7.71-7.67 (m, 1H), 7.44- |
| 430 | | | | | | d6 | 7.38 (m, 2H), 6.11 (s, 1H), 3.43-3.34 (m, 1H), 2.22-2.15 |
| | | | | | | | (m, 2H), 2.07-1.98 (m, 2H), 1.94-1.85 (m, 1H), 1.79- |
| | | | | | | | 1.72 (m, 1H) |
| Cpd | LC-5 | 2.93 | | 423 | 400 | DMSO- | 11.40 (s, 1H), 10.32 (s, 1H), 7.85 (s, 1H), 7.41-7.40 (m, |
| 431 | | | | | | d6 | 1H), 7.19-7.09 (m, 5H), 6.40 (br s, 1H), 3.95 (s, 2H), |
| | | | | | | | 3.84 (s, 3H) |
| Cpd | LC-5 | 2.86 | | 412 | 400 | DMSO- | 11.24 (s, 1H), 10.03 (s, 1H), 7.94-7.93 (m, 1H), 7.58- |
| 432 | | | | | | d6 | 7.54 (m, 1H), 7.26-7.13 (m, 6H), 6.52-6.24 (m, 2H), |
| | | | | | | | 4.43-4.35 (m, 2H), 3.85 (s, 2H) |
| Cpd | LC-5 | 2.87 | | 410 | 400 | DMSO- | 11.28 (s, 1H), 9.33 (s, 1H), 7.64-7.63 (m, 1H), 7.37- |
| 433 | | | | | | d6 | 7.00 (m, 8H), 6.32 (br s, 1H), 3.95 (s, 2H), 3.77 (s, 3H) |
| Cpd | LC-5 | 2.79 | | 398 | 400 | DMSO- | 12.02 (s, 1H), 10.16 (s, 1H), 8.0-7.99 (m, 1H), 7.65- |
| 434 | | | | | | d6 | 7.58 (m, 3H), 7.42-7.37 (m, 3H), 7.26-7.22 (m, 1H), |
| | | | | | | | 6.80-6.79 (m, 1H), 6.52-6.25 (m, 1H), 4.44-4.36 (m, 2H) |
| Cpd | LC-5 | 2.90 | | 409 | 400 | DMSO | 12.09 (s, 1H), 10.61 (s, 1H), 8.01 (s, 1H), 7.64 (d, 2H), |
| 435 | | | | | | d6 | 7.52-7.51 (m, 1H), 7.38 (t, 2H), 7.24 (t, 1H), 6.88 (s, |
| | | | | | | | 1H), 3.90 (s, 3H) |
| Cpd | LC-5 | 3.03 | | 428 | 400 | DMSO- | 11.68 (s, 1H), 9.26 (s, 1H), 7.59 (d, 2H), 7.42-7.33 (m, |
| 436 | | | | | | d6 | 3H), 7.19 (t, 1H), 7.13 (s, 1H), 6.59 (s, 1H), 6.51-6.21 |
| | | | | | | | (m, 1H), 4.54-4.46 (m, 2H), 3.69 (s, 3H) |
| Cpd | LC-5 | 1.63 | | 343 | 400 | DMSO- | 11.55 (br s, 1H), 10.50 (br s, 1H), 8.77 (s, 1H), 8.37- |
| 437 | | | | | | d6 | 8.36 (m, 1H), 8.17-8.13 (br s, 1H), 7.42 (br s, 1H), 7.34- |
| | | | | | | | 7.26 (m, 4H), 7.12 (s, 1H) |
| Cpd | LC-5 | 2.79 | | 416 | 400 | DMSO- | 11.62 (br s, 1H), 10.16 (s, 1H), 7.80 (br s, 1H), 7.58- |
| 438 | | | | | | d6 | 7.37 (m, 4H), 7.31-7.25 (m, 1H), 7.09 (s, 1H), 6.98 (t, |
| | | | | | | | 1H), 6.52-6.23 (m, 1H), 4.33 (t, 2H) |
| Cpd | LC-8 | 4.88 | 388 | | 400 | DMSO- | 11.53 (s, 1H), 10.89 (s, 1H), 7.94 (d, 1H), 7.53-7.48 (m, |
| 439 | | | | | | d6 | 2H), 7.23-7.11 (m, 5H), 6.43 (s, 1H), 3.88 (s, 2H) |
| Cpd | LC-7 | 2.71 | 368 | | 400 | DMSO- | 11.41 (s, 1H), 10.40 (s, 1H), 7.68 (d, 1H), 7.43-7.39 (m, |
| 440 | | | | | | d6 | 2H), 7.25-7.21 (m, 2H), 7.17-7.13 (m, 3H), 6.35 (s, 1H), |
| | | | | | | | 3.87 (s, 2H), 2.36 (s, 3H) |
| Cpd | LC-7 | 2.30 | 330 | | 400 | DMSO- | 11.98 (s, 1H), 10.49 (s, 1H), 7.98 (s, 1H), 7.80 (d, 1H), |
| 441 | | | | | | d6 | 7.68 (t, 1H), 7.61-7.56 (m, 2H), 7.47-7.46 (m, 1H), 6.82 |
| | | | | | | | (s, 1H), 6.55 (s, 1H) |
| Cpd | LC-5 | 2.88 | 380 | | 400 | DMSO- | 12.16 (s, 1H), 10.56 (s, 1H), 8.11-8.10 (m, 1H), 7.74- |
| 442 | | | | | | d6 | 7.53 (m, 6H), 7.28 (t, 1H), 7.12 (s, 1H), 7.03-7.02 (m, 1H) |
| Cpd | LC-5 | 2.89 | 382 | | 400 | DMSO- | 11.84 (s, 1H), 10.49 (s, 1H), 7.77 (d, 1H), 7.58 (br s, |
| 443 | | | | | | d6 | 2H), 7.44-7.42 (m, 2H), 7.13 (d, 1H), 6.86 (t, 2H), 4.64 |
| | | | | | | | (t, 2H), 3.21 (t, 2H) |
| Cpd | LC-5 | 3.09 | | 442 | 400 | DMSO- | 11.22 (s, 1H), 9.16 (s, 1H), 7.48 (d, 1H), 7.28-7.23 (m, |
| 444 | | | | | | d6 | 2H), 7.20-7.15 (m, 3H), 7.11-7.10 (m, 1H), 6.55-6.26 |
| | | | | | | | (m, 2H), 4.64-4.56 (m, 2H), 3.89 (s, 2H), 3.71 (s, 3H) |
| Cpd | LC-5 | 2.64 | 330 | | 400 | DMSO- | 12.22 (s, 1H), 10.53 (s, 1H), 7.80 (d, 1H), 7.66 (s, 1H), |
| 445 | | | | | | d6 | 7.61-7.56 (m, 2H), 7.48-7.47 (m, 1H), 6.68-6.67 (m, |
| | | | | | | | 1H), 6.55-6.53 (m, 2H) |
| Cpd | LC-17 | 1.86 | 429 | | 400 | DMSO- | 12.15 (s, 1H), 10.23 (s, 1H), 7.70 (m, 3H), 7.47 (dd, J = |
| 446 | | | | | | d6 | 3.2, 1.7 Hz, 1H), 7.36 (dd, J = 10.0, 6.9 Hz, 1H), 7.29- |
| | | | | | | | 7.19 (m, 2H), 6.74 (m, 1H) |
| Cpd | LC-17 | 1.89 | 447 | | 400 | DMSO- | 12.14 (s, 1H), 10.24 (s, 1H), 7.82-7.67 (m, 2H), 7.52 |
| 447 | | | | | | d6 | (dd, J = 3.2, 1.7 Hz, 1H), 7.42-7.31 (m, 2H), 7.24- |
| | | | | | | | 7.14 (m, 1H), 6.72 (m, 1H) |
| Cpd | LC-17 | 1.58 | 406 | | 400 | DMSO- | 12.08 (s, 1H), 9.84 (s, 1H), 7.73 (m, 1H), 7.58-7.07 |
| 448 | | | | | | d6 | (m, 6H), 6.73 (m, 1H), 5.22 (s, 2H) |
| Cpd | LC-14 | 1.76 | 440 | | 300 | DMSO- | 12.20 (s, 1H), 9.87 (s, 1H), 7.92-7.83 (m, 1H), 7.44 |
| 449 | | | | | | d6 | (dd, J = 3.1, 1.6 Hz, 1H), 7.41-7.29 (m, 3H), 7.20 (dd, |
| | | | | | | | J = 12.0, 7.3 Hz, 1H), 6.80 (m, 1H), 5.22 (s, 2H) |
| Cpd | LC-17 | 1.61 | 406 | | 400 | DMSO- | 12.09 (s, 1H), 9.81 (s, 1H), 7.73-7.65 (m, 2H), 7.50- |
| 450 | | | | | | d6 | 7.03 (m, 5H), 6.68 (s, 1H), 5.22 (s, 2H) |
| Cpd | LC-17 | 1.65 | 424 | | 400 | DMSO- | 12.09 (s, 1H), 9.86 (s, 1H), 7.77 (m, 1H), 7.43-7.31 |
| 451 | | | | | | d6 | (m, 3H), 7.25-7.14 (m, 2H), 6.68 (m, 1H), 5.23 (s, 2H) |
| Cpd | LC-14 | 1.77 | 419 | | 300 | DMSO- | 11.42 (s, 1H), 10.05 (s, 1H), 7.71-6.72 (m, 7H), 6.51 |
| 452 | | | | | | d6 | (m, 1H), 4.08 (s, 2H) |
| Cpd | LC-14 | 1.83 | 431 | | 300 | DMSO- | 11.43 (s, 1H), 10.08 (s, 1H), 7.66-6.78 (m, 8H), 6.48 |
| 453 | | | | | | d6 | (m, 1H), 3.89 (s, 2H) |
| Cpd | LC-17 | 1.89 | 447 | | 300 | DMSO- | 11.45 (s, 1H), 10.08 (s, 1H), 7.71-6.73 (m, 8H), 6.50 |
| 454 | | | | | | d6 | (m, 1H), 3.89 (s, 2H) |
| Cpd | LC-5 | 2.98 | 415 | | 400 | DMSO- | 12.10 (s, 1H), 10.00 (s, 1H), 7.63 (d, 2H), 7.45 (d, 1H), |
| 455 | | | | | | d6 | 7.41-7.05 (m, 6H), 6.71 (s, 1H) |
| Cpd | LC-5 | 2.99 | 428 | | 400 | DMSO- | 12.30 (s, 1H), 10.93 (s, 1H), 8.02 (t, 1H), 7.94-7.90 (m, |
| 456 | | | | | | d6 | 1H), 7.78 (s, 1H), 7.65 (t, 1H), 7.49-7.45 (m, 1H), 6.85 |
| | | | | | | | (s, 1H) |
| Cpd | LC-5 | 3.10 | 421 | | 400 | DMSO- | 12.03 (s, 1H), 10.68 (s, 1H), 7.72-7.67 (m, 1H), 7.53 (s, |
| 457 | | | | | | d6 | 1H), 7.48-7.43 (m, 2H), 7.12 (s, 1H), 6.60 (s, 1H), 2.43 |
| | | | | | | | (s, 3H) |
| Cpd | LC-5 | 3.15 | 441 | | 400 | DMSO- | 12.15 (s, 1H), 10.71 (s, 1H), 7.72-7.68 (m, 1H), 7.58 (br |
| 458 | | | | | | d6 | s, 2H), 7.51 (s, 1H), 7.48-7.43 (m, 1H), 6.73 (s, 1H) |
| Cpd | LC-5 | 2.82 | 422 | | 400 | DMSO- | 12.00 (s, 1H), 9.76 (s, 1H), 7.54 (d, 2H), 7.42-7.32 (m, |
| 459 | | | | | | d6 | 4H), 7.19-7.16 (m, 1H), 6.69 (s, 1H), 5.21 (s, 2H) |
| Cpd | LC-5 | 2.96 | | 446 | 400 | DMSO- | 11.70 (s, 1H), 9.13 (s, 1H), 7.38-7.24 (m, 5H), 7.09- |
| 460 | | | | | | d6 | 7.08 (m, 1H), 7.01-6.96 (m, 1H), 6.54-6.25 (m, 1H), |
| | | | | | | | 4.58-4.50 (m, 2H), 3.60 (s, 3H) |
| Cpd | LC-5 | 2.78 | 354 | | 400 | DMSO | 11.62 (s, 1H), 10.16 (s, 1H), 7.71-7.68 (m, 1H), 7.47- |
| 461 | | | | | | d6 | 7.44 (m, 1H), 7.37-7.35 (m, 2H), 7.33-7.22 (m, 3H), |
| | | | | | | | 7.20-7.18 (m, 2H), 2.02 (s, 3H) |
| Cpd | LC-5 | 2.89 | 412 | | 400 | DMSO- | 12.07 (s, 1H), 10.92 (s, 1H), 7.94-7.90 (m, 1H), 7.71 (s, |
| 462 | | | | | | d6 | 1H), 7.49-7.45 (m, 1H), 7.34 (t, 2H), 6.69 (s, 1H) |
| Cpd | LC-5 | 2.88 | 413 | | 400 | DMSO- | 12.05 (s, 1H), 9.66 (s, 1H), 7.63 (d, 2H), 7.38 (t, 2H), |
| 463 | | | | | | d6 | 7.28-7.21 (m, 3H), 7.14-7.10 (m, 1H), 6.68 (br s, 1H), |
| | | | | | | | 6.51-6.22 (m, 1H), 4.40-4.32 (m, 2H) |
| Cpd | LC-5 | 2.55 | | 387 | 400 | DMSO- | 11.99 (s, 1H), 9.78 (s, 1H), 7.78 (s, 1H), 7.72 (t, 1H), |
| 464 | | | | | | d6 | 7.55 (s, 1H), 7.30-7.22 (m, 4H), 6.92 (s, 1H), 3.94 (s, |
| | | | | | | | 2H), 3.79 (s, 3H) |
| Cpd | LC-5 | 3.19 | | 454 | 400 | DMSO- | 12.84 (s, 1H), 10.76 (s, 1H), 9.00-8.98 (m, 1H), 8.46- |
| 465 | | | | | | d6 | 8.44 (m, 1H), 8.18-8.16 (m, 1H), 7.92-7.90 (m, 1H), |
| | | | | | | | 7.72-7.68 (m, 1H), 7.66-7.61 (m, 3H), 7.55-7.50 (m, |
| | | | | | | | 1H), 7.29 (s, 1H) |
| Cpd | LC-5 | 2.79 | 406 | | 400 | DMSO- | 11.75 (s, 1H), 9.70 (s, 1H), 7.35-7.26 (m, 4H), 7.22- |
| 466 | | | | | | d6 | 7.17 (m, 1H), 7.12-7.11 (m, 1H), 7.04-6.94 (m, 2H), |
| | | | | | | | 5.17 (s, 2H) |
| Cpd | LC-5 | 2.45 | | 397 | 400 | DMSO- | 12.04 (s, 1H), 9.67 (s, 1H), 7.63-7.62 (m, 2H), 7.38 (t, |
| 467 | | | | | | d6 | 2H), 7.30-7.29 (m, 1H), 7.24 (t, 2H), 7.15 (t, 1H), 7.08- |
| | | | | | | | 7.05 (m, 1H), 6.69-6.68 (m, 1H), 2.13 (s, 3H) |
| Cpd | LC-5 | 2.88 | | 429 | 400 | DMSO- | 11.26 (s, 1H), 9.61 (s, 1H), 7.28-7.03 (m, 8H), 6.52- |
| 468 | | | | | | d6 | 6.25 (m, 2H), 4.40-4.32 (m, 2H), 3.82 (s, 2H) |
| Cpd | LC-5 | 2.82 | | 427 | 400 | DMSO- | 11.79 (s, 1H), 10.56 (s, 1H), 7.84 (s, 1H), 7.51-7.50 (m, |
| 469 | | | | | | d6 | 1H), 7.37-7.32 (m, 3H), 7.09-7.03 (m, 2H), 3.79 (s, 3H) |
| Cpd | LC-5 | 3.06 | 444 | | 400 | DMSO- | 12.33 (s, 1H), 10.92 (s, 1H), 8.05-7.99 (m, 2H), 7.75- |
| 470 | | | | | | d6 | 7.74 (m, 1H), 7.68-7.63 (m, 2H), 6.84-6.83 (m, 1H) |
| Cpd | LC-5 | 3.01 | 424 | | 400 | DMSO- | 12.24 (s, 1H), 10.45 (s, 1H), 8.01 (t, 1H), 7.71 (d, 1H), |
| 471 | | | | | | d6 | 7.67 (s, 1H), 7.65-7.62 (m, 1H), 7.48 (d, 1H), 6.82 (s, |
| | | | | | | | 1H), 2.41 (s, 3H) |
| Cpc | LC-6 | 4.32 | | 373 | 400 | DMSO- | 12.04 (s, 1H), 10.31 (s, 1H), 8.05-8.04 (m, 1H), 7.69- |
| 472 | | | | | | d6 | 7.63 (m, 3H), 7.40-7.37 (m, 3H), 7.24 (t, 1H), 6.81 (s, |
| | | | | | | | 1H), 5.23 (s, 2H) |
| Cpd | LC-6 | 4.76 | | 416 | 400 | DMSO- | 12.23 (s, 1H), 9.71 (s, 1H), 8.54-8.52 (m, 1H), 7.82- |
| 473 | | | | | | d6 | 7.76 (m, 2H), 7.26-7.20 (m, 3H), 7.15-7.10 (m, 1H), |
| | | | | | | | 6.95 (s, 1H), 6.37 (t, 1H), 4.40-4.32 (m, 2H) |
| Cpd | LC-18 | 0.93 | 389 | | 400 | DMSO- | 11.94 (s, 1H), 10.04 (s, 1H), 8.00 (d, J = 1.5 Hz, 1H), |
| 474 | | | | | | d6 | 7.69 (m, 1H), 7.47-6.97 (m, 4H), 6.83 (d, J = 1.9 Hz, |
| | | | | | | | 1H), 6.52 (m, 1H) |
| Cpd | LC-18 | 1.03 | 405 | | 300 | DMSO- | 12.18 (s, 1H), 10.07 (s, 1H), 7.47-7.32 (m, 5H), 7.31- |
| 475 | | | | | | d6 | 6.92 (m, 2H), 6.50 (m, 1H) |
| Cpd | LC-18 | 1.00 | 405 | | 300 | DMSO- | 12.04 (s, 1H), 10.05 (s, 1H), 7.70 (dd, J = 2.9, 1.3 Hz, |
| 476 | | | | | | d6 | 1H), 7.59 (dd, J = 5.0, 2.9 Hz, 1H), 7.47-6.91 (m, 5H), |
| | | | | | | | 6.63 (m, 1H) |
| Cpd | LC-12 | 1.85 | 401 | | 300 | DMSO- | 11.95 (s, 1H), 10.17 (s, 1H), 8.00 (dd, J = 1.6, 0.8 Hz, |
| 477 | | | | | | d6 | 1H), 7.75-7.64 (m, 2H), 7.41 (dd, J = 3.1, 1.7 Hz, 1H), |
| | | | | | | | 7.32 (dd, J = 10.0, 6.9 Hz, 1H), 6.83 (dd, J = 1.9, 0.9 |
| | | | | | | | Hz, 1H), 6.53 (dd, J = 2.5, 1.7 Hz, 1H) |
| Cpd | LC-12 | 1.97 | 417 | | 400 | DMSO- | 12.19 (s, 1H), 10.20 (s, 1H), 7.71 (dd, J = 9.7, 6.4 Hz, |
| 478 | | | | | | d6 | 1H), 7.48-7.41 (m, 2H), 7.38-7.29 (m, 2H), 7.07 (dd, |
| | | | | | | | J = 5.1, 3.6 Hz, 1H), 6.50 (m, 1H). |
| Cpd | LC-12 | 1.77 | 417 | | 300 | DMSO | 12.05 (s, 1H), 10.18 (s, 1H), 7.75-7.63 (m, 2H), 7.59 |
| 479 | | | | | | d6 | (dd, J = 5.0, 2.9 Hz, 1H), 7.48-7.25 (m, 3H), 6.64 (m, 1H) |
| Cpd | LC-12 | 1.95 | 443 | | 400 | DMSO- | 11.37 (s, 1H), 10.06 (s, 1H), 7.56-6.87 (m, 5H), 6.79- |
| 480 | | | | | | d6 | 6.70 (m, 3H), 6.36 (t, J = 2.3 Hz, 1H), 3.83 (s, 2H), 3.70 |
| | | | | | | | (s, 3H) |
| Cpd | LC-17 | 1.89 | 433 | | 300 | DMSO- | 11.58 (s, 1H), 10.75 (s, 1H), 7.64 (dd, J = 10.4, 6.6 Hz, |
| 481 | | | | | | d6 | 1H), 7.53 (dd, J = 3.2, 2.2 Hz, 1H), 7.39-7.17 (m, 2H), |
| | | | | | | | 7.03-6.83 (m, 3H), 6.58 (m, 1H), 3.94 (s, 2H) |
| Cpd | LC-17 | 1.88 | 445 | | 300 | DMSO- | 11.52 (s, 1H), 10.73 (s, 1H), 7.65 (dd, J = 10.4, 6.6 Hz, |
| 482 | | | | | | d6 | 1H), 7.51 (dd, J = 3.2, 2.2 Hz, 1H), 7.34 (dd, J = 12.4, |
| | | | | | | | 6.3 Hz, 1H), 7.12 (m, 1H), 6.70 (m, 3H), 6.48 (m, 1H), |
| | | | | | | | 3.88 (s, 2H), 3.67 (s, 3H) |
| Cpd | LC-14 | 1.85 | 479 | | 400 | DMSO- | 11.42 (s, 1H), 10.07 (s, 1H), 7.44-7.31 (m, 2H), 7.30- |
| 483 | | | | | | d6 | 7.05 (m, 4H), 7.05-7.01 (m, 1H), 7.00-6.92 (m, 2H), |
| | | | | | | | 6.45 (m, 1H), 3.89 (s, 2H) |
| Cpd | LC-14 | 1.80 | 431 | | 400 | DMSO- | 11.39 (s, 1H), 10.09 (s, 1H), 7.43-7.34 (m, 1H), 7.32- |
| 484 | | | | | | d6 | 7.24 (m, 3H), 7.23-7.00 (m, 4H), 6.27 (d, J = 2.5 Hz, |
| | | | | | | | 1H), 3.88 (s, 2H) |
| Cpd | LC-14 | 1.8 | 431 | | 300 | DMSO- | 11.40 (s, 1H), 10.05 (s, 1H), 7.49-6.92 (m, 8H), 6.39 |
| 485 | | | | | | d6 | (s, 1H), 3.86 (s, 2H) |
| Cpd | LC-12 | 1.84 | 374 | | 300 | DMSO- | 11.49 (s, 1H), 10.47 (s, 1H), 7.77 (dd, J = 10.8, 1.7 Hz, |
| 486 | | | | | | d6 | 1H), 7.61-7.44 (m, 2H), 7.39 (dd, J = 3.2, 2.2 Hz, 1H), |
| | | | | | | | 6.62-6.50 (m, 3H), 3.33 (s, 1H), 2.33 (d, J = 1.1 Hz, 3H) |
| Cpd | LC-13 | 1.33 | 408 | | 300 | DMSO- | 12.43 (s, 1H), 10.58 (s, 1H), 7.88 (d, J = 8.2 Hz, 2H), |
| 487 | | | | | | d6 | 7.87-7.78 (m, 1H), 7.74 (d, J = 8.3 Hz, 2H), 7.70- |
| | | | | | | | 7.56 (m, 3H), 6.99 (dd, J = 2.6, 1.7 Hz, 1H) |
| Cpd | LC-12 | 1.95 | 404 | | 400 | DMSO- | 11.96 (s, 1H), 10.54 (s, 1H), 7.83 (dd, J = 10.4, 1.4 Hz, |
| 488 | | | | | | d6 | 1H), 7.70 (d, J = 2.6 Hz, 1H), 7.63 (dd, J = 3.6, 1.4 Hz, |
| | | | | | | | 2H), 7.49 (dd, J = 3.3, 1.8 Hz, 1H), 7.29(dd, J = 8.9, 2.6 |
| | | | | | | | Hz, 1H), 7.12 (d, J = 8.9 Hz, 1H), 6.95 (m, 1H), 3.88 (s, 3H) |
| Cpd | LC-12 | 1.79 | 394 | | 400 | DMSO- | 12.03 (s, 1H), 10.57 (s, 1H), 7.83 (d, J = 10.7 Hz, 1H), |
| 489 | | | | | | d6 | 7.66-7.55 (m, 3H), 7.35 (m, 2H), 6.66 (s, 1H) |
| Cpd | LC-17 | 1.42 | 396 | | 300 | DMSO- | 11.53 (s, 1H), 10.53 (s, 1H), 7.79-7.67 (m, 3H), 7.53 |
| 490 | | | | | | d6 | (dd, J = 8.6, 1.7 Hz, 1H), 7.54-7.43 (m, 1H), 7.46- |
| | | | | | | | 7.36 (m, 2H), 7.41-7.30 (m, 1H), 6.52 (s, 1H), 3.99 (s, 2H) |
| Cpd | LC-5 | 1.98 | | 357 | 400 | DMSO- | 11.59 (s, 1H), 10.36 (s, 1H), 8.48-8.47 (m, 1H), 7.80- |
| 491 | | | | | | d6 | 7.77 (m, 1H), 7.73-7.68 (m, 1H), 7.60-7.52 (m, 2H), |
| | | | | | | | 7.30-7.29 (m, 1H), 7.24-7.21 (m, 1H), 7.13 (d, 1H), 6.06 |
| | | | | | | | (s, 1H), 3.98 (s, 2H) |
| Cpd | LC-6 | 5.54 | 428 | | 400 | DMSO- | 12.05 (s, 1H), 10.87 (s, 1H), 8.99 (d, 1H), 7.64-7.62 (m, |
| 492 | | | | | | d6 | 2H), 7.38-7.32 (m, 2H), 6.66 (s, 1H) |
| Cpd | LC-5 | 2.20 | | 371 | 400 | DMSO- | 11.54 (s, 1H), 10.34 (s, 1H), 8.49-8.48 (m, 1H), 7.80- |
| 493 | | | | | | d6 | 7.78 (m, 1H), 7.72-7.67 (m, 1H), 7.60-7.52 (m, 2H), |
| | | | | | | | 7.27-7.26 (m, 1H), 7.23-7.20 (m, 1H), 7.08 (d, 1H), 6.07 |
| | | | | | | | (s, 1H), 4.17 (q, 1H), 1.50 (d, 3H) |
| Cpd | LC-5 | 2.71 | | 398 | 400 | DMSO- | 12.03 (s, 1H), 10.28 (s, 1H), 7.82 (t, 1H), 7.64 (d, 2H), |
| 494 | | | | | | d6 | 7.41-7.37 (m, 3H), 7.24 (t, 1H), 6.78-6.77 (m, 1H), 4.79- |
| | | | | | | | 4.78 (m, 1H), 4.67-4.66 (m, 1H), 4.39-4.37 (m, 1H), |
| | | | | | | | 4.31-4.29 (m, 1H) |
| Cpd | LC-5 | 2.88 | 408 | | 400 | DMSO- | 11.97 (s, 1H), 10.42 (s, 1H), 7.70 (d. 1H), 7.58 (s, 1H), |
| 495 | | | | | | d6 | 7.47 (d, 1H), 7.33 (t, 2H), 6.65 (s, 1H), 2.40 (s, 3H) |
| Cpd | LC-5 | 2.9 | 431 | | 400 | DMSO- | 11.74 (s, 1H), 9.58 (s, 1H), 7.33-7.26 (m, 4H), 7.13- |
| 496 | | | | | | d6 | 7.08 (m, 2H), 7.03-6.98 (m, 1H), 6.93-6.88 (m, 1H), |
| | | | | | | | 6.52-6.23 (m, 1H), 4.36-4.28 (m, 2H) |
| Cpd | LC-5 | 2.84 | 376 | | 400 | DMSO- | 11.91 (s, 1H), 10.80 (s, 1H), 7.78 (br, 1H), 7.60 (s, 1H), |
| 497 | | | | | | d6 | 7.34-7.25 (m, 3H), 7.21-7.16 (m, 1H), 7.12 (t, 1H), 7.07- |
| | | | | | | | 7.03 (m, 1H) |
| Cpd | LC-5 | 2.76 | | 375 | 400 | DMSO- | 12.17 (s, 1H), 10.52 (s, 1H), 7.80 (d, 1H), 7.69-7.64 (m, |
| 498 | | | | | | d6 | 2H), 7.62-7.57 (m, 2H), 7.41 (s, 1H), 6.98 (s, 1H), 2.46 |
| | | | | | | | (s, 3H) |
| Cpd | LC-5 | 2.76 | | 377 | 400 | DMSO- | 12.45 (s, 1H), 10.58 (s, 1H), 8.57-8.56 (m, 1H), 7.95- |
| 499 | | | | | | d6 | 7.92 (m, 1H), 7.84-7.79 (m, 2H), 7.61-7.60 (m, 2H), |
| | | | | | | | 7.45 (s, 1H), 7.09 (s, 1H) |
| Cpc | LC-5 | 2.33 | | 357 | 400 | DMSO- | 12.30 (s, 1H), 10.54 (s, 1H), 8.37 (s, 1H), 7.81-7.78 (m, |
| 500 | | | | | | d6 | 1H), 7.68-7.66 (m, 1H), 7.63-7.60 (m, 3H), 7.38 (s, 1H), |
| | | | | | | | 6.97 (s, 1H), 2.29 (s, 3H) |
| Cpd | LC-5 | 2.75 | | 412 | 400 | DMSO- | 11.29 (s, 1H), 10.19 (s, 1H), 7.80 (t, 1H), 7.25-7.13 (m, |
| 501 | | | | | | d6 | 6H), 6.26 (s, 1H), 4.80-4.78 (m, 1H), 4.68-4.66 (m, 1H), |
| | | | | | | | 4.38-4.36 (m, 1H), 4.31-4.30 (m, 1H), 3.86 (s, 2H) |
| Cpc | LC-5 | 2.65 | | 361 | 400 | DMSO- | 12.41 (s, 1H), 10.56 (s, 1H), 8.01-7.95 (m, 1H), 7.80- |
| 502 | | | | | | d6 | 7.77 (m, 1H), 7.73-7.71 (m, 1H), 7.59-7.58 (m, 2H), |
| | | | | | | | 7.45 (s, 1H), 7.09 (s, 1H), 7.01-6.99 (m, 1H) |
| Cpd | LC-5 | 2.47 | | 357 | 400 | DMSO- | 12.32 (s, 1H), 10.55 (s, 1H), 8.38-8.37 (m, 1H), 7.81- |
| 503 | | | | | | d6 | 7.78 (m, 1H), 7.64-7.60 (m, 3H), 7.39 (s, 1H), 7.09-7.08 |
| | | | | | | | (m, 1H), 7.01 (s, 1H), 2.33 (s, 3H) |
| Cpd | LC-5 | 2.50 | | 357 | 400 | DMSO- | 12.17 (s, 1H), 10.53 (s, 1H), 7.82-7.79 (m, 1H), 7.67 (t, |
| 504 | | | | | | d6 | 1H), 7.62-7.55 (m, 3H), 7.41-7.40 (m, 1H), 7.10 (d, 1H), |
| | | | | | | | 7.00 (s, 1H), 2.48 (s, 3H) |
| Cpc | LC-5 | 2.77 | 405 | | 400 | DMSO- | 12.49 (s, 1H), 10.78 (s, 1H), 7.86 (s, 1H), 7.74-7.70 (m, |
| 505 | | | | | | d6 | 1H), 7.49-7.44 (m, 1H), 6.98 (s, 1H), 6.42 (s, 1H), 2.88- |
| | | | | | | | 2.87 (m, 2H), 2.38-2.36 (m, 2H) |
| Cpd | LC-17 | 1.69 | 383 | 385 | 400 | DMSO- | 11.91 (s, 1H), 10.45 (s, 1H), 7.84-7.76 (m, 1H), 7.66- |
| 506 | | | | | | d6 | 7.56 (m, 2H), 7.43-7.33 (m, 2H), 7.22 (m, 1H), 7.13 |
| | | | | | | | (d, J = 8.7 Hz, 1H), 7.02 (m, 1H), 6.72 (m, 1H), 2.48 (s, 6H) |
| Cpd | LC-12 | 1.94 | 410 | | 300 | DMSO- | 12.27 (s, 1H), 10.58 (s, 1H), 8.03 (m, 1H), 7.87-7.77 |
| 507 | | | | | | d6 | (m, 1H), 7.72-7.55 (m, 4H), 6.83 (m, 1H) |
| Cpd | LC-19 | 1.93 | 410 | | 400 | DMSO- | 12.37 (s, 1H), 10.97 (s, 1H), 8.15-7.66 (m, 3H), 7.50 |
| 508 | | | | | | d6 | (dd, J = 11.0, 6.4 Hz, 1H), 7.43-7.32 (m, 2H), 6.91 (m, 1H) |
| Cp | LC-19 | 1.79 | 376 | | 400 | DMSO- | 12.26 (s, 1H), 10.94 (s, 1H), 7.93 (dd, J = 10.3, 6.0 Hz, |
| 509 | | | | | | d6 | 1H), 7.71 (m, 3H), 7.51 (dd, J = 11.1, 6.4 Hz, 1H), 7.25 |
| | | | | | | | (m, 2H), 6.82 (m, 1H) |
| Cpd | LC-20 | 1.30 | 394 | | 400 | DMSO- | 12.26 (s, 1H), 10.95 (s, 1H), 7.94 (dd, J = 10.3, 6.0 Hz, |
| 510 | | | | | | d6 | 1H), 7.83-7.73 (m, 2H), 7.50 (dd, J = 11.0, 6.4 Hz, |
| | | | | | | | 1H), 7.38 (m, 1H), 7.24-7.14 (m, 1H), 6.79 (m, 1H) |
| Cp | LC-19 | 1.79 | 372 | | 400 | DMSO- | 12.19 (s, 1H), 10.47 (s, 1H), 7.78-7.70 (m, 2H), 7.65 |
| 511 | | | | | | d6 | (dd, J = 3.2, 1.7 Hz, 1H), 7.52 (d, J = 7.7 Hz, 1H), 7.39- |
| | | | | | | | 7.22 (m, 3H), 6.81 (m, 1H), 2.42 (s, 3H) |
| Cpc | LC-20 | 1.66 | 406 | | 400 | DMSO- | 12.30 (s, 1H), 10.49 (s, 1H), 7.91-7.84 (m, 1H), 7.77- |
| 512 | | | | | | d6 | 7.68 (m, 2H), 7.50 (d, J = 7.8 Hz, 1H), 7.40-7.31 (m, |
| | | | | | | | 2H), 6.88 (m, 1H), 2.42 (s, 3H) |
| Cpd | LC-19 | 1.80 | 372 | | 300 | DMSO- | 12.18 (s, 1H), 10.44 (s, 1H), 7.77-7.63 (m, 3H), 7.58 |
| 513 | | | | | | d6 | (dd, J = 3.2, 1.7 Hz, 1H), 7.51 (d, J = 7.7 Hz, 1H), 7.31- |
| | | | | | | | 7.17 (m, 2H), 6.78 (dd, J = 2.6, 1.7 Hz, 1H), 2.42 (s, 3H) |
| Cpd | LC-20 | 1.54 | 390 | | 400 | DMSO- | 12.19 (s, 1H), 10.47 (s, 1H), 7.82-7.70 (m, 2H), 7.65 |
| 514 | | | | | | d6 | (dd, J = 3.2, 1.7 Hz, 1H), 7.51 (d, J = 7.8 Hz, 1H), 7.37 |
| | | | | | | | (m, 1H), 7.19 (m, 1H), 6.76 (m, 1H), 2.42 (s, 3H) |
| Cpc | LC-21 | 1.35 | | 436 | 300 | DMSO- | 12.35 (s, 1H), 10.14 (s, 1H), 7.97-7.68 (m, 2H), 7.37 |
| 515 | | | | | | d6 | (m, 4H), 7.26-6.83 (m, 2H) |
| Cpd | LC-14 | 1.81 | | 480 | 300 | DMSO- | 12.32 (s, 1H), 10.13 (s, 1H), 7.83 (dd, J = 7.8, 1.0 Hz, |
| 516 | | | | | | d6 | 1H), 7.75 (m, 1H), 7.52-7.27 (m, 4H), 7.24-6.91 (m, 2H) |
| Cpd | LC-10 | 3.54 | 441 | | 400 | DMSO- | 12.32 (s, 1H), 10.76 (s, 1H), 8.11-8.10 (m, 1H), 7.72- |
| 517 | | | | | | d6 | 7.66 (m, 3H), 7.60-7.57 (m, 1H), 7.53-7.49 (m, 1H), |
| | | | | | | | 7.30 (t, 1H), 7.17 (s, 1H), 7.04-7.03 (m, 1H) |
| Cpd | LC-5 | 3.12 | 437 | | 400 | DMSO- | 11.90 (s, 1H), 10.67 (s, 1H), 7.71-7.67 (m, 1H), 7.65- |
| 518 | | | | | | d6 | 7.64 (m, 1H), 7.54 (s, 1H), 7.48-7.44 (m, 1H), 6.74 (s, |
| | | | | | | | 1H), 6.70-6.69 (m, 1H), 3.84 (s, 3H) |
| Cpd | LC-5 | 3.1 | 443 | | 400 | DMSO- | 11.91 (s, 1H), 10.68 (s, 1H), 7.73-7.68 (m, 1H), 7.56 (s, |
| 519 | | | | | | d6 | 1H), 7.50-7.44 (m, 2H), 7.13 (d, 1H), 6.90-6.85 (m, 2H), |
| | | | | | | | 4.63 (t, 2H), 3.21 (t, 2H) |
| Cpd | LC-5 | 2.85 | 407 | | 400 | DMSO- | 11.64 (s, 1H), 10.59 (s, 1H), 7.72-7.68 (m, 1H), 7.46 (s, |
| 520 | | | | | | d6 | 1H), 7.45-7.40 (m, 1H), 6.15 (s, 1H), 3.37-3.31 (m, 1H), |
| | | | | | | | 2.47-2.46 (m, 1H), 2.28-2.18 (m, 4H), 1.89-1.82 (m, 1H) |
| Cpd | LC-22 | 1.40 | | 385 | 400 | DMSO- | 12.10 (s, 1H), 10.49 (s, 1H), 7.80 (dd, J = 10.9, 1.6 Hz, |
| 521 | | | | | | d6 | 1H), 7.67-7.56 (m, 2H), 7.49 (dd, J = 3.2, 1.7 Hz, 1H), |
| | | | | | | | 7.18 (m, 1H), 6.96 (m, 1H), 6.91 (m, 1H), 6.74 (dd, J = |
| | | | | | | | 2.6, 1.7 Hz, 1H), 6.62 (dd, J = 8.1, 2.5 Hz, 1H), 2.93 (s, 6H) |
| Cpd | LC-23 | 1.13 | 392 | | 400 | DMSO- | 12.28 (s, 1H), 10.93 (s, 1H), 8.02 (d, J = 10.4 Hz, 1H), |
| 522 | | | | | | d6 | 7.88-7.51 (m, 3H), 7.51-7.01 (m, 3H), 6.83 (m, 1H) |
| Cpd | LC-23 | 1.35 | 426 | | 400 | DMSO- | 12.36 (s, 1H), 10.93 (s, 1H), 8.00 (d, J = 10.4 Hz, 1H), |
| 523 | | | | | | d6 | 7.92-7.85 (m, 1H), 7.74 (dd, J = 3.2, 1.7 Hz, 1H), 7.66 |
| | | | | | | | (d, J = 7.0 Hz, 1H), 7.43-7.31 (m, 2H), 6.89 (m, 1H) |
| Cpd | LC-23 | 1.14 | 392 | | 300 | DMSO- | 12.26 (s, 1H), 10.90 (s, 1H), 8.00 (d, J = 10.4 Hz, 1H), |
| 524 | | | | | | d6 | 7.76-7.60 (m, 4H), 7.31-7.17 (m, 2H), 6.79 (m, 1H) |
| Cpd | LC-24 | 1.12 | 433 | | 400 | DMSO- | 12.07 (s, 1H), 10.08 (s, 1H), 7.55 (m, 2H), 7.45 (dd, J = |
| 526 | | | | | | d6 | 3.2, 1.7 Hz, 1H), 7.44-7.34 (m, 4H), 7.32-7.01 (m, |
| | | | | | | | 1H), 6.74 (dd, J = 2.6, 1.7 Hz, 1H) |
| Cpd | LC-12 | 2.06 | | 448 | 300 | DMSO- | 12.26 (s, 1H), 10.90 (s, 1H), 8.00 (d, J = 10.4 Hz, 1H), |
| 527 | | | | | | d6 | 7.76-7.60 (m, 4H), 7.31-7.17 (m, 2H), 6.79 (m, 1H) |
| Cpd | LC-12 | 2.09 | | 492 | 400 | DMSO- | 12.33 (s, 1H), 10.28 (s, 1H), 7.83 (d, J = 7.7 Hz, 1H), |
| 528 | | | | | | d6 | 7.79-7.66 (m, 2H), 7.48 (d, J = 7.7 Hz, 1H), 7.42 (dd, |
| | | | | | | | J = 3.2, 1.7 Hz, 1H), 7.34 (dd, J = 10.0, 6.8Hz, 1H), |
| | | | | | | | 7.08 (m, 1H) |
| Cpd | LC-23 | 1.15 | 368 | | 400 | DMSO- | 12.20 (s, 1H), 10.46 (s, 1H), 7.72 (d, J = 10.6 Hz, 1H), |
| 529 | | | | | | d6 | 7.64 (d, J = 7.7 Hz, 2H), 7.58-7.53 (m, 1H), 7.50 (d, J = |
| | | | | | | | 7.8 Hz, 1H), 7.39 (m, 2H), 7.26(m, 1H), 6.80 (m, 1H), |
| | | | | | | | 2.73 (m, 2H), 1.15 (m, 3H) |
| Cpd | LC-23 | 1.26 | 429 | | 400 | DMSO- | 11.75 (s, 1H), 10.07 (s, 1H), 7.59 (dd, J = 7.7, 1.6 Hz, |
| 530 | | | | | | d6 | 1H), 7.44-7.31 (m, 3H), 7.29-7.25 (m, 1H), 7.23 (d, |
| | | | | | | | J = 15.3 Hz, 1H), 7.10 (dd, J = 8.4, 1.1 Hz, 1H), 7.04- |
| | | | | | | | 6.96 (m, 1H), 6.79 (dd, J = 2.6, 1.8 Hz, 1H), 3.87 (s, 3H) |
| Cpd | LC-23 | 1.31 | 447 | | 400 | DMSO- | 11.94 (s, 1H), 10.12 (s, 1H), 7.48-7.30 (m, 4H), 7.23- |
| 531 | | | | | | d6 | 7.00 (m, 3H), 6.85 (m, 1H), 3.78 (d, J = 1.5 Hz, 3H) |
| Cpd | LC-24 | 1.21 | 441 | | 300 | DMSO- | 11.77 (s, 1H), 10.21 (s, 1H), 7.71 (dd, J = 9.7, 6.4 Hz, |
| 532 | | | | | | d6 | 1H), 7.60 (dd, J = 7.7, 1.7 Hz, 1H), 7.41-7.22 (m, 3H), |
| | | | | | | | 7.10 (dd, J = 8.3, 1.1 Hz, 1H), 6.99 (m, 1H), 6.80 (dd, J = |
| | | | | | | | 2.7, 1.8 Hz, 1H), 3.87 (s, 3H) |
| Cpd | LC-24 | 1.19 | 445 | | 400 | DMSO- | 12.08 (s, 1H), 10.20 (s, 1H), 7.71 (dd, J = 9.7, 6.4 Hz, |
| 533 | | | | | | d6 | 1H), 7.55 (m, 2H), 7.49 (dd, J = 3.2, 1.7 Hz, 1H), 7.41 |
| | | | | | | | (m, 1H), 7.39-7.29 (m, 2H), 6.74 (dd, J = 2.6, 1.7 Hz, 1H) |
| Cpd | LC-18 | 1.39 | 462 | | 300 | DMSO- | 12.82 (s, 1H), 10.28 (s, 1H), 9.00 (dd, J = 4.2, 1.8 Hz, |
| 534 | | | | | | d6 | 1H), 8.45 (dd, J = 8.4, 1.8 Hz, 1H), 8.17 (dd, J = 7.4, |
| | | | | | | | 1.4 Hz, 1H), 7.91 (dd, J = 8.2, 1.4 Hz, 1H), 7.75-7.56 |
| | | | | | | | (m, 3H), 7.49 (dd, J = 3.2, 1.7 Hz, 1H), 7.39 (dd, J = |
| | | | | | | | 10.0, 6.9 Hz, 1H), 7.24 (m, 1H) |
| Cpd | LC-23 | 1.02 | 390 | | 300 | DMSO- | 11.56 (s, 1H), 10.53 (s, 1H), 7.76 (dd, J = 10.8, 1.8 Hz, |
| 535 | | | | | | d6 | 1H), 7.60-7.39 (m, 3H), 7.24 (m, 1H), 7.11 (m, 1H), |
| | | | | | | | 6.97(s, 1H), 6.60 (m, 1H), 3.90 (s, 2H) |
| Cpd | LC-23 | 0.91 | 402 | | 300 | DMSO- | 11.50 (s, 1H), 10.53 (s, 1H), 7.76 (dd, J = 10.8, 1.8 Hz, |
| 536 | | | | | | d6 | 1H), 7.59-7.44 (m, 2H), 7.41 (dd, J = 3.2, 2.2 Hz, 1H), |
| | | | | | | | 7.07-6.91 (m, 2H), 6.66 (m, 1H), 6.49 (m, 1H), 3.87 |
| | | | | | | | (s, 2H), 3.75 (s, 3H) |
| Cpd | LC-5 | 2.61 | 363 | | 400 | DMSO- | 12.07 (s, 1H), 9.94 (s, 1H), 7.63 (d, 2H), 7.40-7.37 (m, |
| 537 | | | | | | d6 | 3H), 7.27-7.23 (m, 1H), 7.21-7.17 (m, 1H), 7.14-7.09 |
| | | | | | | | (m, 1H), 6.72 (s, 1H), 5.27 (t, 1H), 4.44-4.43 (m, 2H) |
| Cpd | LC-5 | 2.89 | | 424 | 400 | DMSO- | 12.23 (s, 1H), 10.70 (s, 1H), 7.82 (s, 1H), 7.63-7.57 (m, |
| 538 | | | | | | d6 | 2H), 7.44-7.39 (m, 1H), 6.54 (s, 1H), 2.63 (s, 3H) |
| Cpd | LC-6 | 6.30 | 437 | | 400 | DMSO- | 11.70 (s, 1H), 10.66 (s, 1H), 7.72-7.68 (m, 1H), 7.47- |
| 539 | | | | | | d6 | 7.44 (m, 2H), 7.19-7.18 (m, 1H), 6.88-6.87 (m, 1H), |
| | | | | | | | 6.61 (s, 1H), 3.95 (s, 3H) |
| Cpc | LC-5 | 3.10 | | 457 | 400 | DMSO- | 12.11 (s, 1H), 10.76 (s, 1H), 8.41 (s, 1H), 7.72-7.66 (m, |
| 540 | | | | | | d6 | 1H), 7.64-7.60 (m, 3H), 7.53-7.49 (m, 1H), 7.43 (s, 1H), |
| | | | | | | | 7.07 (t, 1H), 4.23 (s, 3H) |
| Cpc | LC-5 | 2.95 | 436 | | 400 | DMSO- | 12.29 (s, 1H), 10.23 (s, 1H), 8.15 (s, 1H), 7.99 (d, 1H), |
| 541 | | | | | | d6 | 7.72-7.67 (m, 2H), 7.58 (t, 1H), 7.53 (s, 1H), 7.36-7.32 |
| | | | | | | | (m, 1H), 6.96 (s, 1H) |
| Cpd | LC-5 | 2.96 | 414 | | 400 | DMSO- | 11.90 (s, 1H), 10.53 (s, 1H), 7.77 (d, 1H), 7.58 (s, 2H), |
| 542 | | | | | | d6 | 7.46 (s, 1H), 7.38 (d, 1H), 7.23-7.15 (m, 2H), 6.74 (s, |
| | | | | | | | 1H), 3.99-3.97 (m, 1H), 0.46-0.43 (m, 4H) |
| Cpd | LC-5 | 2.73 | 377 | | 400 | DMSO- | 12.41 (s, 1H), 10.55 (s, 1H), 8.53-8.52 (m, 1H), 8.07- |
| 543 | | | | | | d6 | 8.02 (m, 1H), 7.78 (d, 1H), 7.61-7.58 (m, 2H), 7.47 (s, |
| | | | | | | | 1H), 6.91 (s, 1H) |
| Cpd | LC-25 | 1.10 | | 461 | 400 | DMSO- | 11.95 (s, 1H), 10.24 (s, 1H), 7.71 (dd, J = 9.6, 6.4 Hz, |
| 544 | | | | | | d6 | 1H), 7.48-7.41 (m, 2H), 7.36 (dd, J = 9.9, 6.8 Hz, 1H), |
| | | | | | | | 7.25-7.10 (m, 2H), 6.86 (m, 1H), 3.78 (d, J = 1.6 Hz, 3H) |
| Cpd | LC-23 | 1.12 | 406 | | 300 | DMSO- | 11.57 (s, 1H), 10.54 (s, 1H), 7.74 (dd, J = 10.8, 1.8 Hz, |
| 545 | | | | | | d6 | 1H), 7.58-7.39 (m, 3H), 7.30-7.08 (m, 3H), 6.62 (m, |
| | | | | | | | 1H), 3.90 (s, 2H) |
| Cpd | LC-23 | 1.02 | 390 | | 300 | DMSO- | 11.60 (s, 1H), 10.55 (s, 1H), 7.74 (dd, J = 10.9, 1.8 Hz, |
| 546 | | | | | | d6 | 1H), 7.60-7.39 (m, 3H), 6.96 (m, 1H), 6.85-6.72 (m, |
| | | | | | | | 2H), 6.68 (m, 1H), 3.94 (s, 2H) |
| Cpd | LC-23 | 0.99 | 402 | | 300 | DMSO- | 11.54 (s, 1H), 10.53 (s, 1H), 7.74 (dd, J = 10.8, 1.8 Hz, |
| 547 | | | | | | d6 | 1H), 7.58-7.45 (m, 2H), 7.42 (dd, J = 3.2, 2.2 Hz, 1H), |
| | | | | | | | 6.63-6.52 (m, 2H), 6.55-6.42 (m, 2H), 3.87 (s, 2H), |
| | | | | | | | 3.69 (s, 3H) |
| Cpd | LC-17 | 1.69 | 438 | | 300 | DMSO- | 11.62 (s, 1H), 10.94 (s, 1H), 7.84 (dd, J = 10.3, 6.0 Hz, |
| 548 | | | | | | d6 | 1H), 7.60 (m, 1H), 7.46-6.71 (m, 6H), 6.59 (m, 1H), |
| | | | | | | | 3.94 (s, 2H) |
| Cpd | LC-5 | 2.84 | 336 | | 400 | DMSO- | 11.58 (s, 1H), 10.76 (s, 1H), 7.92-7.88 (m, 1H), 7.44- |
| 549 | | | | | | d6 | 7.39 (m, 2H), 6.13 (s, 1H), 3.43-3.34 (m, 1H), 2.22-2.15 |
| | | | | | | | (m, 2H), 2.08-1.98 (m, 2H), 1.94-1.82 (m, 1H), 1.79- |
| | | | | | | | 1.76 (m, 1H) |
| Cpd | LC-5 | 2.93 | | 379 | 400 | DMSO- | 11.44 (s, 1H), 9.89 (s, 1H), 7.40-7.35 (m, 1H), 7.28- |
| 550 | | | | | | d6 | 7.24 (m, 1H), 7.19 (t, 1H), 7.17 (br s, 1H), 6.04 (s, 1H), |
| | | | | | | | 3.42-3.34 (m, 1H), 2.23-2.16 (m, 2H), 2.06-1.77 (m, 4H) |
| Cpd | LC-5 | 3.00 | | 391 | 400 | DMSO- | 11.46 (s, 1H), 10.04 (s, 1H), 7.70-7.66 (m, 1H), 7.30- |
| 551 | | | | | | d6 | 7.21 (m, 1H), 7.21 (s, 1H), 6.06 (s, 1H), 3.42-3.34 (m, |
| | | | | | | | 1H), 2.23-2.16 (m, 2H), 2.08-2.02 (m, 2H), 2.00-1.79 |
| | | | | | | | (m, 2H) |
| Cpd | LC-5 | 3.04 | | 452 | 400 | DMSO- | 12.78 (s, 1H), 10.11 (s, 1H), 8.99-8.98 (m, 1H), 8.46- |
| 552 | | | | | | d6 | 8.44 (m, 1H), 8.15 (d, 1H), 7.90 (d, 1H), 7.65-7.61 (m, |
| | | | | | | | 2H), 7.44 (br s, 1H), 7.41-7.00 (m, 4H) |
| Cpd | LC-5 | 2.99 | 439 | | 400 | DMSO- | 12.24 (s, 1H), 10.12 (s, 1H), 8.10 (s, 1H), 7.66 (d, 1H), |
| 553 | | | | | | d6 | 7.58 (d, 1H), 7.49 (br s, 1H), 7.42-7.00 (m, 6H) |
| Cpd | LC-17 | 1.84 | 444 | | 300 | DMSO- | 12.38 (s, 1H), 10.81 (s, 1H), 8.30 (dd, J = 7.3, 2.1 Hz, |
| 554 | | | | | | d6 | 1H), 7.90-7.66 (m, 3H), 7.53 (m, 2H), 6.95 (m, 1H) |
| Cpd | LC-17 | 1.60 | 432 | | 300 | DMSO- | 12.11 (s, 1H), 10.76 (s, 1H), 7.91 (dd, J = 7.2, 1.9 Hz, |
| 555 | | | | | | d6 | 1H), 7.72 (m, 2H), 7.54-7.41 (m, 2H), 7.14 (m, 1H), |
| | | | | | | | 6.36 (m, 1H), 3.53 (s, 3H) |
| Cpd | LC-17 | 1.66 | 436 | | 300 | DMSO- | 12.35 (s, 1H), 10.77 (s, 1H), 8.75 (s, 1H), 8.46 (d, J = |
| 556 | | | | | | d6 | 5.3 Hz, 1H), 7.80-7.69 (m, 2H), 7.64 (d, J = 5.3 Hz, |
| | | | | | | | 1H), 7.52 (dd, J = 12.2, 6.3 Hz, 1H), 6.95-6.88 (m, 1H) |
| Cpd | LC-17 | 1.72 | 438 | | 300 | DMSO- | 12.12 (s, 1H), 10.80 (s, 1H), 8.23 (dd, J = 8.1, 5.6 Hz, |
| 557 | | | | | | d6 | 1H), 7.79-7.67 (m, 2H), 7.55-7.43 (m, 2H), 6.83 (m, 1H) |
| Cpd | LC-17 | 1.93 | 481 | | 400 | MeOD- | 7.45 (d, J = 2.2 Hz, 1H), 7.35 (m, 2H), 7.20 (m, 1H), |
| 558 | | | | | | d4 | 6.98 (d, J = 7.6 Hz, 1H), 6.92-6.41 (m, 4H), 4.01 (s, 2H) |
| Cpd | LC-5 | 2.89 | 408 | | 400 | DMSO- | 12.31 (s, 1H), 10.74 (s, 1H), 9.14 (s, 1H), 8.94 (s, 1H), |
| 559 | | | | | | d6 | 7.74-7.67 (m, 2H), 7.51-7.46 (m, 1H), 6.88 (s, 1H) |
| Cpd | LC-5 | 2.99 | | 448 | 400 | DMSO- | 12.27 (s, 1H), 10.72 (s, 1H), 8.56-8.55 (m, 1H), 7.88 (d, |
| 560 | | | | | | d6 | 1H), 7.75-7.71 (m, 1H), 7.55-7.50 (m, 2H), 7.35-7.31 |
| | | | | | | | (m, 1H), 6.82 (bs, 1H), 4.46 (s, 2H), 3.33 (s, 3H) |
| Cpd | LC-5 | 3.03 | 451 | | 400 | DMSO- | 12.23 (s, 1H), 10.24 (s, 1H), 8.10 (s, 1H), 7.70-7.65 (m, |
| 561 | | | | | | d6 | 2H), 7.59-7.57 (m, 1H), 7.52 (bs, 1H), 7.37-7.28 (m, |
| | | | | | | | 2H), 7.12 (bs, 1H), 7.039-7.034 (m, 1H) |
| Cpd | LC-5 | 2.87 | 380 | | 400 | DMSO- | 12.12 (s, 1H), 10.51 (s, 1H), 7.81-7.79 (d, 1H), 7.59- |
| 562 | | | | | | d6 | 7.56 (m, 3H), 7.50-7.48 (m, 2H), 6.70 (s, 1H) |
| Cpd | LC-5 | 2.85 | 380 | | 400 | DMSO- | 12.29 (s, 1H), 10.52 (s, 1H), 7.82-7.79 (d, 1H), 7.61- |
| 563 | | | | | | d6 | 7.59 (m, 2H), 7.56-7.53 (m, 1H), 7.48-7.47 (m, 1H), |
| | | | | | | | 7.31-7.30 (m, 1H), 6.62 (s, 1H) |
| Cpd | LC-17 | 1.81 | 444 | | 300 | DMSO- | 12.42 (s, 1H), 10.83 (s, 1H), 8.08 (m, 1H), 7.89-7.68 |
| 564 | | | | | | d6 | (m, 3H), 7.57-7.41 (m, 2H), 6.92 (m, 1H) |
| Cpd | LC-12 | 1.45 | 441 | | 400 | DMSO- | 12.53 (s, 1H), 10.83 (s, 1H), 8.52 (d, J = 6.7 Hz, 1H), |
| 565 | | | | | | d6 | 8.05 (s, 1H), 7.78-7.67 (m, 4H), 7.63-7.45 (m, 2H), |
| | | | | | | | 7.00 (m, 1H) |
| Cpd | LC-26 | 1.81 | 441 | | 400 | DMSO- | 12.74 (s, 1H), 10.89 (s, 1H), 8.19 (d, J = 2.4 Hz, 1H), |
| 566 | | | | | | d6 | 7.84 (d, J = 2.9 Hz, 2H), 7.79-7.69 (m, 2H), 7.64- |
| | | | | | | | 7.46 (m, 2H), 7.35 (m, 1H), 6.79 (d, J = 2.4 Hz, 1H) |
| Cpd | LC-26 | 1.54 | 441 | | 400 | DMSO- | 12.02 (s, 1H), 10.66 (s, 1H), 8.21 (dd, J = 4.4, 1.7 Hz, |
| 567 | | | | | | d6 | 1H), 8.11 (dd, J = 9.2, 1.7 Hz, 1H), 7.91 (d, J = 2.9 Hz, |
| | | | | | | | 1H), 7.73 (dd, J = 10.3, 6.6 Hz, 1H), 7.61(dd, J = 3.1, |
| | | | | | | | 1.7 Hz, 1H), 7.56 (dd, J = 12.3, 6.3 Hz, 1H), 7.18 (d, J = |
| | | | | | | | 2.9 Hz, 1H), 6.77 (dd, J = 9.2, 4.4 Hz, 1H), 6.65 (m, 1H) |
| Cpd | LC-26 | 1.81 | 449 | | 400 | DMSO- | 11.60 (s, 1H), 10.76 (s, 1H), 7.64 (dd, J = 10.3, 6.6 Hz, |
| 568 | | | | | | d6 | 1H), 7.55 (m, 1H), 7.33 (dd, J = 12.3, 6.3 Hz, 1H), 7.23 |
| | | | | | | | (m, 1H), 7.17 (m, 1H), 7.13 (d, J = 7.3 Hz, 1H), 7.09 (m, |
| | | | | | | | 1H), 6.61 (m, 1H), 3.94 (s, 2H) |
| Cpd | LC-26 | 1.48 | 406 | | 400 | DMSO- | 11.65 (s, 1H), 10.94 (s, 1H), 7.84 (dd, J = 10.3, 5.9 Hz, |
| 569 | | | | | | d6 | 1H), 7.60 (m, 1H), 7.30 (dd, J = 11.1, 6.4 Hz, 1H), 7.23 |
| | | | | | | | (m, 1H), 7.17 (d, J = 8.0 Hz, 1H), 7.11 (d, J = 7.5 Hz, |
| | | | | | | | 1H), 7.06 (d, J = 2.0 Hz, 1H), 6.65 (m, 1H), 3.94 (s, 2H) |
| Cpd | LC-6 | 5.17 | 364 | | 400 | DMSO- | 12.23 (s, 1H), 10.49 (s, 1H), 7.80 (d, 1H), 7.61-7.55 (m, |
| 570 | | | | | | d6 | 2H), 7.49-7.48 (m, 1H), 6.98 (t, 1H), 6.72-6.70 (m, 1H), |
| | | | | | | | 6.48 (s, 1H) |
| Cpd | LC-5 | 3.22 | 407 | | 400 | DMSO- | 11.52 (s, 1H), 10.54 (s, 1H), 7.71-7.67 (m, 1H), 7.44- |
| 571 | | | | | | d6 | 7.38 (m, 2H), 6.09 (s, 1H), 3.39-3.32 (m, 1H), 2.03-1.98 |
| | | | | | | | (m, 2H), 1.84-1.79 (m, 2H), 1.15 (s, 3H), 1.06 (s, 3H) |
| Cpd | LC-5 | 2.92 | 414 | | 400 | DMSO- | 12.40 (s, 1H), 10.98 (s, 1H), 7.94-7.83 (m, 3H), 7.77 (br |
| 572 | | | | | | d6 | s, 1H), 7.64 (d, 1H), 7.55 (d, 1H), 7.52-7.46 (m, 2H), |
| | | | | | | | 7.00 (s, 1H) |
| Cpd | LC-6 | 5.79 | | 436 | 400 | DMSO- | 12.38 (s, 1H), 10.71 (s, 1H), 8.65-8.64 (m, 1H), 7.98 (d, |
| 573 | | | | | | d6 | 1H), 7.74-7.70 (m, 1H), 7.58 (s, 1H), 7.53-7.49 (m, 1H), |
| | | | | | | | 7.41-7.38 (m, 1H), 6.83 (s, 1H), 5.54 (d, 2H) |
| Cpd | LC-17 | 1.78 | 440 | | 300 | DMSO- | 11.64 (s, 1H), 10.77 (s, 1H), 7.77-7.16 (m, 7H), 6.68 |
| 574 | | | | | | d6 | (m, 1H), 4.00 (s, 2H) |
| Cpd | LC-27 | 0.94 | 393 | | 400 | DMSO- | 12.49 (s, 1H), 10.97 (s, 1H), 7.94 (dd, J = 10.3, 6.0 Hz, |
| 575 | | | | | | d6 | 1H), 7.88 (m, 1H), 7.82 (dd, J = 7.8, 1.0 Hz, 1H), 7.64 |
| | | | | | | | (dd, J = 3.3, 1.7 Hz, 1H), 7.49 (dd, J = 11.0, 6.4 Hz, |
| | | | | | | | 1H), 7.36 (dd, J = 7.6, 0.9 Hz, 1H), 7.17 (dd, J = 2.6, |
| | | | | | | | 1.7 Hz, 1H) |
| Cpd | LC-17 | 1.59 | 404 | | 300 | DMSO- | 12.10 (s, 1H), 9.78 (s, 1H), 7.65 (m, 2H), 7.45-7.21 |
| 576 | | | | | | d6 | (m, 6H), 6.73-6.66 (m, 1H), 5.26 (s, 2H) |
| Cpd | LC-5 | 2.74 | | 375 | 400 | DMSO- | 12.19 (s, 1H), 10.48 (s, 1H), 8.45-8.44 (m, 1H), 7.81 (d, |
| 577 | | | | | | d6 | 1H), 7.72 (dd, 1H), 7.62-7.61 (m, 2H), 7.42-7.41 (m, |
| | | | | | | | 1H), 6.77 (s, 1H) 2.44 (s, 3H) |
| Cpd | LC-5 | 2.74 | 366 | | 400 | DMSO- | 11.39 (s, 1H), 9.60 (s, 1H), 7.34-7.30 (m, 1H), 7.14- |
| 578 | | | | | | d6 | 7.08 (m, 2H), 6.01 (s, 1H), 5.21 (s, 2H), 3.40-3.36 (m, |
| | | | | | | | 1H), 2.23-2.16 (m, 2H), 2.05-1.97 (m, 2H), 1.92-1.91 |
| | | | | | | | (m, 1H), 1.89-1.87 (m, 1H) |
| Cpd | LC-5 | 2.49 | 288 | | 400 | DMSO- | 12.15 (s, 1H), 11.14 (s, 1H), 8.70 (s, 1H), 7.64 (d, 2H), |
| 579 | | | | | | d6 | 7.48 (s, 1H), 7.38 (t, 2H), 7.25 (t, 1H), 6.76 (s, 1H), 6.50 |
| | | | | | | | (s, 1H) |
| Cpd | LC-5 | 3.06 | | 454 | 400 | DMSO- | 12.47 (s, 1H), 10.71 (s, 1H), 8.76 (d, 1H), 8.13 (d, 1H), |
| 580 | | | | | | d6 | 7.74-7.70 (m, 1H), 7.62 (s, 1H), 7.53-7.47 (m, 2H), 7.14 |
| | | | | | | | (t, 1H), 6.81 (s, 1H) |
| Cpd | LC-5 | 2.73 | | 393 | 400 | DMSO- | 12.37 (s, 1H), 10.53 (s, 1H), 8.75 (d, 1H), 8.12 (d, 1H), |
| 581 | | | | | | d6 | 7.75 (br, 1H), 7.57 (s, 2H), 7.49-7.46 (m, 2H), 7.13 (t, |
| | | | | | | | 1H), 6.78 (s, 1H) |
| Cpd | LC-5 | 2.94 | | 422 | 400 | DMSO- | 11.85 (s, 1H), 10.63 (s, 1H), 7.75-7.71 (m, 1H), 7.62- |
| 582 | | | | | | d6 | 7.60 (m, 1H), 7.50-7.45 (m, 1H), 6.46-6.45 (m, 1H), |
| | | | | | | | 2.35 (s, 3H), 2.17 (s, 3H) |
| Cpd | LC-5 | 2.22 | | 290 | 400 | DMSO- | 11.87 (s, 1H), 11.68 (br s, 1H), 7.64 (d, 2H), 7.57 (s, |
| 583 | | | | | | d6 | 1H), 7.40-7.36 (m, 3H), 7.25-7.21 (m, 2H), 6.78 (s, 1H) |
| Cpd | LC-17 | 1.66 | 390 | | 400 | DMSO- | 11.58 (s, 1H), 10.92 (s, 1H), 7.86 (dd, J = 10.3, 6.0 Hz, |
| 584 | | | | | | d6 | 1H), 7.57 (dd, J = 3.3, 2.2 Hz, 1H), 7.31 (dd, J = 11.1, |
| | | | | | | | 6.4 Hz, 1H), 7.20-7.11 (m, 2H), 7.07-6.97 (m, 2H), |
| | | | | | | | 6.51 (m, 1H), 3.90 (s, 2H) |
| Cpd | LC-17 | 1.65 | 390 | | 400 | DMSO- | 11.62 (s, 1H), 10.95 (s, 1H), 7.85 (dd, J = 10.3, 6.0 Hz, |
| 585 | | | | | | d6 | 1H), 7.59 (dd, J = 3.2, 2.2 Hz, 1H), 7.35-7.19 (m, 2H), |
| | | | | | | | 7.00-6.91 (m, 2H), 6.88 (m, 1H), 6.61 (m, 1H), 3.94 |
| | | | | | | | (s, 2H) |
| Cpd | LC-17 | 1.83 | 412 | | 400 | DMSO- | 11.54 (s, 1H), 10.93 (s, 1H), 7.86 (dd, J = 10.3, 5.9 Hz, |
| 586 | | | | | | d6 | 1H), 7.56 (m, 1H), 7.31 (m, 1H), 7.07 (m, 1H), 6.89 (dd, |
| | | | | | | | J = 7.8, 1.6 Hz, 1H), 6.81 (dd, J = 6.5, 1.5 Hz, 2H), 6.46 |
| | | | | | | | (m, 1H), 3.86 (s, 2H), 1.77 (m, 1H), 0.95-0.82 (m, |
| | | | | | | | 2H), 0.61-0.52 (m, 2H) |
| Cpd | LC-27 | 1.05 | 424 | | 400 | DMSO- | 12.21 (s, 1H), 10.57 (s, 1H), 7.85-7.77 (m, 1H), 7.76- |
| 587 | | | | | | d6 | 7.69 (m, 1H), 7.66-7.55 (m, 3H), 7.45 (m, 3H), 6.71 |
| | | | | | | | (m, 1H) |
| Cpd | LC-28 | 1.28 | 442 | | 400 | DMSO- | 12.29 (s, 1H), 10.96 (s, 1H), 7.93 (dd, J = 10.2, 5.9 Hz, |
| 588 | | | | | | d6 | 1H), 7.79-7.71 (m, 2H), 7.52-7.41 (m, 4H), 6.74 (m, 1H) |
| Cpd | LC-27 | 1.45 | 485 | | 400 | DMSO- | 12.26 (s, 1H), 10.75 (s, 1H), 7.77-7.65 (m, 3H), 7.52- |
| 589 | | | | | | d6 | 7.45 (m, 1H), 7.48-7.40 (m, 3H), 6.71 (m, 1H) |
| Cpd | LC-27 | 1.53 | 455 | | 300 | DMSO- | 11.50 (s, 1H), 10.75 (s, 1H), 7.67 (dd, J = 10.3, 6.6 Hz, |
| 590 | | | | | | d6 | 1H), 7.55-7.47 (m, 1H), 7.34 (dd, J = 12.4, 6.3 Hz, |
| | | | | | | | 1H), 7.08 (m, 1H), 6.95-6.74 (m, 3H), 6.42 (m, 1H), |
| | | | | | | | 3.85 (s, 2H), 1.78 (m, 1H), 0.94-0.82 (m, 2H), 0.62- |
| | | | | | | | 0.51 (m, 2H) |
| Cpd | LC-5 | 2.56 | | 306 | 400 | DMSO- | 12.08 (s, 1H), 10.32 (br s, 1H), 8.39 (s, 1H), 8.28 (s, |
| 591 | | | | | | d6 | 1H), 7.62 (d, 2H), 7.40-7.36 (m, 3H), 7.24 (t, 1H), 6.70 |
| | | | | | | | (s, 1H) |
| Cpd | LC-11 | 5.43 | | 375 | 400 | DMSO- | 12.35 (s, 1H), 10.53 (s, 1H), 8.64-8.63 (m, 1H), 7.99- |
| 592 | | | | | | d6 | 7.97 (m, 1H), 7.83-7.80 (m, 1H), 7.62-7.61 (m, 2H), |
| | | | | | | | 7.48 (s, 1H), 7.41-7.38 (m, 1H), 6.81 (s, 1H), 5.54 (d, 2H) |
| Cpd | LC-6 | 5.11 | | 323 | 400 | DMSO- | 12.13 (s, 1H), 10.50 (s, 1H), 7.64 (d, 2H), 7.41-7.35 (m, |
| 593 | | | | | | d6 | 3H), 7.25 (t, 1H), 6.70 (s, 1H), 6.64 (s, 1H), 6.47 (s, 1H) |
| Cpd | LC-5 | 2.74 | 364 | | 400 | DMSO- | 12.18 (s, 1H), 10.52 (s, 1H), 7.82 (d, 1H), 7.71-7.69 (m, |
| 594 | | | | | | d6 | 1H), 7.63-7.58 (m, 2H), 7.56 (s, 1H), 7.33-7.32 (m, 1H), |
| | | | | | | | 6.60 (s, 1H) |
| Cpd | LC-5 | 2.97 | | 424 | 400 | DMSO- | 12.22 (s, 1H), 10.73 (s, 1H), 7.67-7.65 (m, 2H), 7.51- |
| 595 | | | | | | d6 | 7.44 (m, 2H), 6.74 (s, 1H), 2.67 (s, 3H) |
| Cpd | LC-5 | 3.00 | 410 | | 400 | DMSO- | 12.31 (s, 1H), 10.93 (s, 1H), 7.94-7.90 (m, 2H), 7.70- |
| 596 | | | | | | d6 | 7.66 (m, 2H), 7.50-7.42 (m, 2H), 6.94 (s, 1H) |
| Cpd | LC-5 | 2.83 | | 410 | 400 | DMSO- | 12.41 (s, 1H), 10.73 (s, 1H), 9.00 (s, 1H), 8.14 (s, 1H), |
| 597 | | | | | | d6 | 7.74-7.68 (m, 2H), 7.50-7.45 (m, 1H), 6.69 (s, 1H) |
| Cpd | LC-12 | 1.48 | 365 | | 400 | DMSO- | 12.77 (s, 1H), 10.96 (s, 1H), 7.94 (dd, J = 10.2, 5.9 Hz, |
| 598 | | | | | | d6 | 1H), 7.84 (d, J = 3.3 Hz, 1H), 7.73-7.66 (m, 2H), 7.51 |
| | | | | | | | (dd, J = 11.0, 6.4 Hz, 1H), 6.97 (s, 1H) |
| Cpd | LC-29 | 2.10 | 422 | | 400 | DMSO- | 12.69 (s, 1H), 10.75 (s, 1H), 7.73 (dd, J = 10.3, 6.6 Hz, |
| 599 | | | | | | d6 | 1H), 7.58 (m, 1H), 7.49 (dd, J = 12.2, 6.3 Hz, 1H), 7.23 |
| | | | | | | | (s, 1H), 6.90 (d, J = 2.2 Hz, 1H), 2.38 (s, 3H) |
| Cpd | LC-5 | 2.46 | | 373 | 400 | DMSO- | 12.06 (s, 1H), 10.50 (s, 1H), 7.89-7.87 (m, 1H), 7.81- |
| 600 | | | | | | d6 | 7.78 (m, 1H), 7.72-7.70 (m, 1H), 7.60-7.57 (m, 2H), |
| | | | | | | | 7.39 (s, 1H), 7.08 (s, 1H), 6.34 (t, 1H), 3.52 (s, 3H) |
| Cpd | LC-5 | 2.78 | 346 | | 400 | DMSO- | 12.23 (s, 1H), 11.04 (br, 1H), 7.88 (s, 1H), 7.67 (d, 2H), |
| 601 | | | | | | d6 | 7.42-7.38 (m, 3H), 7.26 (t, 1H), 6.76 (s, 1H) |
| Cpd | LC-5 | 2.74 | 364 | | 400 | DMSO- | 12.14 (s, 1H), 10.52 (s, 1H), 7.83-7.80 (m, 1H), 7.60- |
| 602 | | | | | | d6 | 7.57 (m, 2H), 7.51-7.49 (m, 1H), 7.47 (s, 1H), 7.07-7.06 |
| | | | | | | | (m, 1H), 6.55 (s, 1H) |
| Cpd | LC-5 | 2.69 | 399 | | 400 | DMSO- | 12.08 (s, 1H), 10.52 (s, 1H), 7.86-7.73 (m, 3H), 7.60- |
| 603 | | | | | | d6 | 7.55 (m, 2H), 7.38 (s, 1H), 7.07 (s, 1H), 6.41 (t, 1H), |
| | | | | | | | 5.22-5.15 (m, 1H), 1.32 (d, 6H) |
| Cpd | LC-5 | 2.65 | 407 | | 400 | DMSO- | 12.05 (s, 1H), 10.55 (s, 1H), 8.10-7.76 (m, 4H), 7.63- |
| 604 | | | | | | d6 | 7.56 (m, 2H), 7.49-7.48 (m, 1H), 7.17 (s, 1H), 6.54 (t, 1H) |
| Cpd | LC-17 | 1.76 | 424 | | 300 | DMSO- | 11.70 (s, 1H), 10.95 (s, 1H), 7.84 (dd, J = 10.4, 6.0 Hz, |
| 605 | | | | | | d6 | 1H), 7.63 (dd, J = 3.2, 2.1 Hz, 1H), 7.27 (dd, J = 11.2, |
| | | | | | | | 6.5 Hz, 1H), 7.14 (m, 1H), 6.96-6.85 (m, 2H), 6.77 (m, |
| | | | | | | | 1H), 3.96 (s, 2H) |
| Cpd | LC-17 | 1.77 | 422 | | 300 | DMSO- | 12.65 (s, 1H), 10.78 (s, 1H), 7.73 (dd, J = 10.3, 6.6 Hz, |
| 606 | | | | | | d6 | 1H), 7.60 (dd, J = 3.1, 1.7 Hz, 1H), 7.56-7.44 (m, 2H), |
| | | | | | | | 6.85 (m, 1H), 2.45 (d, J = 1.2 Hz, 3H) |
| Cpd | LC-17 | 1.72 | 433 | | 300 | DMSO- | 13.10 (s, 1H), 10.86 (s, 1H), 8.66 (s, 1H), 7.82-7.69 |
| 607 | | | | | | d6 | (m, 2H), 7.51 (dd, J = 12.1, 6.3 Hz, 1H), 7.25 (d, J = 1.6 |
| | | | | | | | Hz, 1H) |
| Cpd | LC-29 | 1.81 | 396 | | 300 | DMSO- | 11.45 (s, 1H), 10.47 (s, 1H), 7.76 (dd, J = 10.8, 1.7 Hz, |
| 608 | | | | | | d6 | 1H), 7.58-7.43 (m, 2H), 7.37 (dd, J = 3.2, 2.2 Hz, 1H), |
| | | | | | | | 7.04 (d, J = 7.4 Hz, 1H), 6.60 (dd, J = 7.5.1.5 Hz, 1H), |
| | | | | | | | 6.48 (d, J = 1.4 Hz, 1H), 6.41 (m, 1H), 4.47 (m, 2H), |
| | | | | | | | 3.81 (s, 2H), 3.10 (m, 2H) |
| Cpd | LC-30 | 1.45 | 372 | | 300 | DMSO- | 12.43 (s, 1H), 10.66 (s, 1H), 7.77 (dd, J = 10.8, 1.8 Hz, |
| 609 | | | | | | d6 | 1H), 7.60-7.43 (m, 2H), 7.26-7.07 (m, 6H), 3.86 (s, 2H) |
| Cpd | LC-29 | 1.42 | 347 | | 300 | DMSO- | 12.72 (s, 1H), 10.59 (s, 1H), 7.88-7.78 (m, 2H), 7.70 |
| 610 | | | | | | d6 | (d, J = 3.2 Hz, 1H), 7.67-7.55 (m, 2H), 7.51 (dd, J = |
| | | | | | | | 3.2, 1.7 Hz, 1H), 6.91 (dd, J = 2.5, 1.7 Hz, 1H) |
| Cpd | LC-5 | 2.49 | 391 | | 400 | DMSO- | 13.02 (br s, 1H), 10.23 (br s, 1H), 9.26 (s, 1H), 7.54 (s, |
| 611 | | | | | | d6 | 1H), 7.37-6.99 (m, 4H) |
| Cpd | LC-5 | 2.72 | 341 | | 400 | DMSO- | 12.06 (s, 1H), 10.51 (s, 1H), 7.71 (d, 1H), 7.62 (d, 2H), |
| 612 | | | | | | d6 | 7.59-7.51 (m, 2H), 7.43 (s, 1H), 7.37 (d, 2H), 6.75 (s, 1H) |
| Cpd | LC-5 | 2.66 | 407 | | 400 | DMSO- | 12.96 (br s, 1H), 10.21 (br s, 1H), 8.82 (s, 1H), 7.57 (s, |
| 613 | | | | | | d6 | 1H), 7.40-7.32 (m, 2H), 7.18-6.99 (m, 2H) |
| Cpd | LC-5 | 2.65 | 439 | | 400 | DMSO- | 12.04 (s, 1H), 10.53 (s, 1H), 7.94 (d, 1H), 7.80 (d, 1H), |
| 614 | | | | | | d6 | 7.69 (d, 1H), 7.61-7.56 (m, 2H), 7.43 (s, 1H), 7.14 (s, |
| | | | | | | | 1H), 6.46 (t, 1H), 5.00-4.94 (m, 2H) |
| Cpd | LC-5 | 2.92 | 398 | | 400 | DMSO- | 12.26 (s, 1H), 10.95 (s, 1H), 8.11-8.10 (m, 1H), 7.81 |
| 615 | | | | | | d6 | (br, 1H), 7.69 (br, 1H), 7.66 (d, 1H), 7.57 (d, 1H), 7.46- |
| | | | | | | | 7.42 (m, 1H), 7.29 (t, 1H), 7.16 (s, 1H), 7.04-7.03 (m, 1H) |
| Cpd | LC-17 | 1.79 | 360 | | 300 | DMSO- | 11.41 (s, 1H), 10.53 (s, 1H), 7.79 (dd, J = 10.8, 1.7 Hz, |
| 616 | | | | | | d6 | 1H), 7.66-7.43 (m, 2H), 7.35 (dd, J = 3.2, 2.2 Hz, 1H), |
| | | | | | | | 6.61 (m, 1H), 2.38 (d, J = 7.1Hz, 2H), 1.59 (d, J = 8.8 |
| | | | | | | | Hz, 5H), 1.51-1.30 (m, 1H), 1.09 (s, 3H), 0.81 (d, J = |
| | | | | | | | 11.9 Hz, 2H) |
| Cpd | LC-31 | 1.36 | 431 | | 400 | DMSO- | 13.00 (s, 1H), 10.22 (s, 1H), 8.66 (s, 1H), 7.59-7.01 |
| 617 | | | | | | d6 | (m, 5H) |
| Cpd | LC-31 | 1.31 | 403 | | 400 | DMSO- | 12.63 (s, 1H), 10.82 (s, 1H), 9.13 (d, J = 1.6 Hz, 1H), |
| 618 | | | | | | d6 | 8.59 (dd, J = 2.6, 1.5 Hz, 1H), 8.49 (d, J = 2.6 Hz, 1H), |
| | | | | | | | 7.74 (dd, J = 10.3, 6.6 Hz, 1H), 7.66 (dd, J = 3.2, 1.7 |
| | | | | | | | Hz, 1H), 7.52 (dd, J = 12.1, 6.3 Hz, 1H), 7.30 (dd, J = |
| | | | | | | | 2.6, 1.7 Hz, 1H) |
| Cpd | LC-31 | 1.18 | 403 | | 400 | DMSO- | 12.63 (s, 1H), 10.82 (s, 1H), 9.13 (d, J = 1.6 Hz, 1H), |
| 619 | | | | | | d6 | 8.59 (dd, J = 2.6, 1.5 Hz, 1H), 8.49 (d, J = 2.6 Hz, 1H), |
| | | | | | | | 7.74 (dd, J = 10.3, 6.6 Hz, 1H), 7.66 (dd, J = 3.2, 1.7 |
| | | | | | | | Hz, 1H), 7.52 (dd, J = 12.1, 6.3 Hz, 1H), 7.30 (dd, J = |
| | | | | | | | 2.6, 1.7 Hz, 1H) |
| Cpd | LC-5 | 2.55 | | 396 | 400 | DMSO- | 12.18 (s, 1H), 10.55 (s, 1H), 8.32 (s, 1H), 7.77-7.75 (m, |
| 620 | | | | | | d6 | 1H), 7.64-7.56 (m, 3H), 7.52-7.46 (m, 2H), 7.32-7.27 |
| | | | | | | | (m, 2H), 3.87 (s, 3H) |
| Cpd | LC-5 | 3.02 | 429 | | 400 | DMSO- | 11.64 (s, 1H), 10.59 (s, 1H), 7.73-7.68 (m, 1H), 7.45- |
| 621 | | | | | | d6 | 7.40 (m, 2H), 6.17 (s, 1H), 3.26-3.23 (m, 1H), 2.48-2.41 |
| | | | | | | | (m, 1H), 2.24-2.06 (m, 4H), 1.78-1.73 (m, 1H) |
| Cpd | LC-5 | 2.69 | 318 | | 400 | DMSO- | 11.41 (s, 1H), 10.38 (s, 1H), 7.79-7.76 (m, 1H), 7.58- |
| 622 | | | | | | d6 | 7.56 (m, 2H), 7.27 (s, 1H), 6.10 (s, 1H), 2.38-2.37 (m, |
| | | | | | | | 2H), 0.90-0.88 (m, 1H), 0.43-0.41 (m, 2H), 0.11-0.10 |
| | | | | | | | (m, 2H) |
| Cpd | LC-32 | 1.09 | 355 | | 300 | DMSO- | 11.40 (s, 1H), 10.60 (s, 1H), 7.85-7.75 (m, 1H), 7.62- |
| 623 | | | | | | d6 | 7.48 (m, 2H), 7.37 (dd, J = 3.2, 2.2 Hz, 1H), 7.33-7.09 |
| | | | | | | | (m, 5H), 6.66 (m, 1H), 2.81 (s, 4H) |
| Cpd | LC-32 | 0.92 | 355 | | 400 | DMSO- | 11.57 (s, 1H), 10.58 (s, 1H), 8.41-8.35 (m, 2H), 7.76 |
| 624 | | | | | | d6 | (dd, J = 10.8, 1.8 Hz, 1H), 7.60-7.38 (m, 3H), 7.16- |
| | | | | | | | 7.10 (m, 2H), 6.60 (m, 1H), 3.94 (s, 2H) |
| Cpd | LC-17 | 1.50 | 368 | | 300 | DMSO- | 11.40 (s, 1H), 10.60 (s, 1H), 7.85-7.75 (m, 1H), 7.62- |
| 625 | | | | | | d6 | 7.48 (m, 2H), 7.37 (dd, J = 3.2, 2.2 Hz, 1H), 7.33-7.09 |
| | | | | | | | (m, 5H), 6.66 (m, 1H), 2.81 (s, 4H) |
| Cpd | LC-27 | 1.29 | 419 | | 300 | DMSO- | 12.25 (s, 1H), 11.09 (s, 1H), 7.75 (dd, J = 10.2, 6.6 Hz, |
| 626 | | | | | | d6 | 1H), 7.63-7.54 (m, 2H), 7.59-7.38 (m, 4H), 7.35- |
| | | | | | | | 7.23 (m, 1H) |
| Cpd | LC-27 | 1.29 | 419 | | 300 | DMSO- | 13.05 (s, 1H), 11.00 (s, 1H), 7.72 (dd, J = 10.2, 6.6 Hz, |
| 627 | | | | | | d6 | 1H), 7.59-7.50 (m, 2H), 7.49-7.31 (m, 3H), 7.36- |
| | | | | | | | 7.19 (m, 1H), 6.65 (d, J = 3.7 Hz, 1H) |
| Cpd | LC-31 | 1.08 | 406 | | 400 | DMSO- | 12.35 (s, 1H), 10.10 (s, 1H), 9.00 (d, J = 0.8 Hz, 1H), |
| 628 | | | | | | d6 | 8.14 (d, J = 0.8 Hz, 1H), 7.48 (dd, J = 3.1, 1.7 Hz, 1H), |
| | | | | | | | 7.46-7.32 (m, 2H), 7.32-7.00 (m, 1H), 6.61 (dd, J = |
| | | | | | | | 2.5, 1.7 Hz, 1H) |
| Cpd | LC-31 | 1.19 | 406 | | 400 | DMSO- | 12.23 (s, 1H), 10.11 (s, 1H), 9.15 (d, J = 1.9 Hz, 1H), |
| 629 | | | | | | d6 | 7.89 (d, J = 1.9 Hz, 1H), 7.45-6.99 (m, 4H), 6.77 (dd, |
| | | | | | | | J = 2.5, 1.7 Hz, 1H) |
| Cpd | LC-5 | 2.78 | | 374 | 400 | DMSO- | 13.12 (br, 1H), 12.04 (s, 1H), 8.11 (br, 1H), 7.67 (d, |
| 630 | | | | | | d6 | 2H), 7.44 (s, 1H), 7.37 (t, 2H), 7.23 (t, 1H), 6.78 (s, 1H) |
| Cpd | LC-5 | 2.54 | | 331 | 400 | DMSO- | 13.30 (br, 1H), 12.07 (s, 1H), 8.33 (s, 1H), 7.67 (d, 2H), |
| 631 | | | | | | d6 | 7.43 (s, 1H), 7.38 (t, 2H), 7.24 (t, 1H), 6.78 (s, 1H) |
| Cpd | LC-17 | 1.58 | 406 | | 400 | DMSO- | 12.06 (s, 1H), 10.08 (s, 1H), 7.56 (m, 2H), 7.43 (dd, J = |
| 632 | | | | | | d6 | 8.5, 7.1 Hz, 2H), 7.38-7.19 (m, 3H), 7.16 (d, J = 3.4 |
| | | | | | | | Hz, 1H), 5.20 (s, 2H) |
| Cpd | LC-17 | 1.65 | | 438 | 400 | DMSO- | 12.06 (s, 1H), 10.07 (s, 1H), 7.44-7.30 (m, 3H), 7.30- |
| 633 | | | | | | d6 | 7.01 (m, 2H), 6.64 (m, 1H), 4.07 (s, 3H) |
| Cpc | LC-17 | 1.58 | 420 | | 300 | DMSO- | 12.62 (s, 1H), 10.14 (s, 1H), 7.46-7.32 (m, 3H), 7.32- |
| 634 | | | | | | d6 | 6.95 (m, 2H), 6.81 (dd, J = 2.5, 1.7 Hz, 1H), 2.39 (d, J = |
| | | | | | | | 1.0 Hz, 3H) |
| Cpd | LC-17 | 1.51 | 406 | | 400 | DMSO- | 12.43 (s, 1H), 10.12 (s, 1H), 9.10 (d, J = 4.7 Hz, 1H), |
| 635 | | | | | | d6 | 7.78 (d, J = 4.7 Hz, 1H), 7.45-7.32 (m, 3H), 7.32- |
| | | | | | | | 7.01 (m, 1H), 6.94 (dd, J = 2.6, 1.7 Hz, 1H) |
| Cpd | LC-17 | 1.37 | | 392 | 300 | DMSO- | 12.77 (s, 1H), 10.21 (s, 1H), 8.15 (d, J = 0.8 Hz, 1H), |
| 636 | | | | | | d6 | 7.48-6.94 (m, 5H), 6.87 (m, 1H) |
| Cpd | LC-33 | 1.52 | 474 | | 400 | DMSO- | 12.95 (s, 1H), 10.22 (s, 1H), 8.45 (s, 1H), 7.54 (d, J = |
| 637 | | | | | | d6 | 1.7 Hz, 1H), 7.46-7.00 (m, 4H) |
| Cpd | LC-17 | 1.46 | 406 | | 300 | DMSO- | 12.54 (s, 1H), 10.15 (s, 1H), 8.51 (d, J = 1.8 Hz, 1H), |
| 638 | | | | | | d6 | 7.64 (d, J = 1.8 Hz, 1H), 7.57 (dd, J = 2.9, 1.6 Hz, 1H), |
| | | | | | | | 7.47-7.33 (m, 2H), 7.33-6.95 (m, 1H), 6.82 (d, J = |
| | | | | | | | 1.9 Hz, 1H) |
| Cpd | LC-17 | 1.45 | 406 | | 300 | DMSO- | 12.24 (s, 1H), 10.10 (s, 1H), 9.14 (s, 1H), 8.94 (s, 1H), |
| 639 | | | | | | d6 | 7.50-7.33 (m, 3H), 7.32-6.95 (m, 1H), 6.81 (m, 1H) |
| Cpd | LC-34 | 1.71 | 348 | | 300 | DMSO- | 12.07 (s, 1H), 10.91 (s, 1H), 8.01 (m, 1H), 7.92 (dd, J = |
| 640 | | | | | | d6 | 10.3, 6.0 Hz, 1H), 7.73-7.61 (m, 2H), 7.47 (dd, J = |
| | | | | | | | 11.1, 6.5 Hz, 1H), 6.85 (d, J = 2.1 Hz, 1H), 6.60 (m, 1H) |
| Cpd | LC-17 | 1.13 | | 349 | 300 | DMSO- | 12.41 (s, 1H), 10.55 (s, 1H), 9.00 (d, J = 0.7 Hz, 1H), |
| 641 | | | | | | d6 | 8.14 (d, J = 0.8 Hz, 1H), 7.88-7.78 (m, 1H), 7.67- |
| | | | | | | | 7.55 (m, 3H), 6.67 (dd, J = 2.5, 1.7 Hz, 1H) |
| Cpd | LC-17 | 1.29 | | 367 | 300 | DMSO- | 12.37 (s, 1H), 10.94 (s, 1H), 9.16 (d, J = 1.9 Hz, 1H), |
| 642 | | | | | | d6 | 7.98-7.87 (m, 2H), 7.63 (dd, J = 3.2, 1.7 Hz, 1H), 7.49 |
| | | | | | | | (dd, J = 11.1, 6.4 Hz, 1H), 6.84 (m, 1H) |
| Cpc | LC-5 | 2.94 | 392 | | 400 | DMSO- | 11.60 (s, 1H), 10.92 (s, 1H), 7.86-7.82 (m, 1H), 7.57- |
| 643 | | | | | | d6 | 7.56 (m, 1H), 7.32-7.20 (m, 2H), 6.98-6.89 (m, 3H), |
| | | | | | | | 6.60 (s, 1H) |
| Cpd | LC-5 | 2.90 | 346 | | 400 | DMSO- | 12.14 (s, 1H), 8.85-8.32 (m, 1H), 7.66 (d, 2H), 7.50 (s, |
| 644 | | | | | | d6 | 1H), 7.39 (t, 2H), 7.25 (t, 1H), 6.76 (s, 1H), 6.37 (s, 1H) |
| Cpd | LC-9 | 5.33 | 401 | | 400 | DMSO- | 12.31 (br s, 1H), 10.95 (s, 1H), 8.29-8.27 (m, 1H), 7.92- |
| 645 | | | | | | d6 | 7.77 (m, 3H), 7.57-7.51 (m, 1H), 7.45-7.42 (br, 1H), |
| | | | | | | | 6.92 (s, 1H) |
| Cpd | LC-33 | 1.33 | 365 | | 300 | DMSO- | 12.57 (s, 1H), 10.96 (s, 1H), 9.11 (d, J = 4.7 Hz, 1H), |
| 646 | | | | | | d6 | 7.93 (dd, J = 10.3, 6.0 Hz, 1H), 7.80 (d, J = 4.7 Hz, 1H), |
| | | | | | | | 7.64 (dd, J = 3.2, 1.7 Hz, 1H), 7.49 (dd, J = 11.0, 6.5 |
| | | | | | | | Hz, 1H), 7.04 (dd, J = 2.5, 1.7 Hz, 1H) |
| Cpd | LC-17 | 1.24 | | 367 | 300 | DMSO- | 12.47 (s, 1H), 10.92 (s, 1H), 9.01 (d, J = 0.8 Hz, 1H), |
| 647 | | | | | | d6 | 8.15 (d, J = 0.7 Hz, 1H), 7.94 (dd, J = 10.3, 6.0 Hz, 1H), |
| | | | | | | | 7.75 (dd, J = 3.1, 1.7 Hz, 1H), 7.49 (dd, J = 11.0, 6.5 |
| | | | | | | | Hz, 1H), 6.71 (dd, J = 2.5, 1.7 Hz, 1H) |
| Cpd | LC-35 | 1.44 | 399 | | 400 | DMSO- | 12.84 (s, 1H), 10.96 (s, 1H), 7.94 (dd, J = 10.3, 6.0 Hz, |
| 648 | | | | | | d6 | 1H), 7.87 (s, 1H), 7.72 (dd, J = 3.3, 1.7 Hz, 1H), 7.50 |
| | | | | | | | (dd, J = 10.9, 6.4 Hz, 1H), 7.01 (dd, J = 2.5, 1.7 Hz, 1H) |
| Cpd | LC-33 | 1.38 | | 381 | 400 | DMSO- | 12.73 (s, 1H), 10.93 (s, 1H), 7.93 (dd, J = 10.3, 6.0 Hz, |
| 649 | | | | | | d6 | 1H), 7.63 (dd, J = 3.2, 1.7 Hz, 1H), 7.49 (dd, J = 11.0, |
| | | | | | | | 6.4 Hz, 1H), 7.24 (d, J = 1.2 Hz, 1H), 6.91 (m, 1H), 2.39 |
| | | | | | | | (d, J = 1.0 Hz, 3H) |
| Cpd | LC-5 | 2.46 | 442 | | 400 | DMSO- | 12.25 (s, 1H), 10.15 (s, 1H), 8.29-8.28 (m, 1H), 7.84- |
| 650 | | | | | | d6 | 7.81 (m, 1H), 7.57-7.52 (m, 2H), 7.40-7.00 (m, 3H), |
| | | | | | | | 6.85 (s, 1H) |
| Cpd | LC-5 | 2.93 | 398 | | 400 | DMSO- | 12.26 (s, 1H), 10.87 (s, 1H), 8.29 (s, 1H), 7.95-7.90 (m, |
| 651 | | | | | | d6 | 1H), 7.83-7.81 (m, 1H), 7.77-7.76 (m, 1H), 7.66-7.64 |
| | | | | | | | (m, 1H), 7.54-7.50 (m, 1H), 7.41-7.38 (m, 2H), 6.86 (bs, 1H) |
| Cpd | LC-5 | 2.94 | 406 | | 400 | DMSO- | 11.98 (s, 1H), 10.93 (s, 1H), 7.92-7.86 (m, 1H), 7.60 (s, |
| 652 | | | | | | d6 | 1H), 7.48-7.43 (m, 2H), 7.21-7.11 (m, 2H), 6.92 (s, 1H), |
| | | | | | | | 3.79 (s, 3H) |
| Cpd | LC-5 | 3.04 | 405 | | 400 | DMSO- | 11.84 (s, 1H), 10.64 (s, 1H), 7.73-7.69 (m, 1H), 7.55 (s, |
| 653 | | | | | | d6 | 1H), 7.53 (s, 1H), 7.50-7.45 (m, 1H), 6.69 (s, 1H), 6.38 |
| | | | | | | | (s, 1H), 2.35 (s, 3H) |
| Cpd | LC-5 | 2.92 | 376 | | 400 | DMSO- | 12.26 (s, 1H), 10.92 (s, 1H), 7.88-7.84 (m, 1H), 7.66 (s, |
| 654 | | | | | | d6 | 1H), 7.54-7.39 (m, 4H), 7.09-7.05 (m, 1H), 6.92 (s, 1H) |
| Cpd | LC-5 | 1.92 | 448 | | 400 | DMSO- | 12.19 (s, 1H), 10.36 (s, 1H), 7.88-7.83 (m, 2H), 7.50 (s, |
| 655 | | | | | | d6 | 1H), 7.36 (d, 2H), 6.91 (s, 1H), 4.80-4.78 (m, 1H), 4.68- |
| | | | | | | | 4.66 (m, 1H), 4.40-4.39 (m, 1H), 4.32-4.31 (m, 1H) |
| Cpd | LC-33 | 1.27 | 365 | | 400 | DMSO- | 12.65 (s, 1H), 10.98-10.93 (m, 1H), 8.51 (d, J = 1.8 |
| 656 | | | | | | d6 | Hz, 1H), 7.94 (dd, J = 10.3, 5.9 Hz, 1H), 7.82 (dd, J = |
| | | | | | | | 3.2, 1.7 Hz, 1H), 7.65 (d, J = 1.8 Hz, 1H), 7.50 (dd, J = |
| | | | | | | | 11.0, 6.4 Hz, 1H), 6.93 (m, 1H) |
| Cpd | LC-33 | 1.27 | 365 | | 400 | DMSO- | 12.36 (s, 1H), 10.96 (s, 1H), 9.15 (d, J = 2.3 Hz, 1H), |
| 657 | | | | | | d6 | 8.95 (d, J = 1.3 Hz, 1H), 7.93 (dd, J = 10.3, 6.0 Hz, 1H), |
| | | | | | | | 7.73 (s, 1H), 7.48 (dd, J = 11.0, 6.3 Hz, 1H), 6.92- |
| | | | | | | | 6.85 (m, 1H) |
| Cpd | LC-17 | 1.23 | | 351 | 300 | DMSO- | 12.89 (s, 1H), 10.98 (s, 1H), 8.16 (d, J = 0.8 Hz, 1H), |
| 658 | | | | | | d6 | 7.94 (dd, J = 10.3, 6.0 Hz, 1H), 7.72 (dd, J = 3.2, 1.7 |
| | | | | | | | Hz, 1H), 7.49 (dd, J = 11.0, 6.4 Hz, 1H), 7.34 (d, J = 0.8 |
| | | | | | | | Hz, 1H), 6.95 (dd, J = 2.5, 1.7 Hz, 1H) |
| Cpd | LC-17 | 1.75 | | 421 | 400 | DMSO- | 12.10 (s, 1H), 10.05 (s, 1H), 7.45-7.19 (m, 4H), 7.14 |
| 659 | | | | | | d6 | (d, J = 1.4 Hz, 1H), 7.02 (m, 1H), 6.45 (m, 1H), 2.20 (d, |
| | | | | | | | J = 1.1 Hz, 3H) |
| Cpd | LC-17 | 1.67 | | 437 | 400 | DMSO | 12.15 (s, 1H), 10.08 (s, 1H), 7.44-7.37 (m, 2H), 7.36- |
| 660 | | | | | | d6 | 7.29 (m, 1H), 7.21 (s, 1H), 7.03-7.00 (m, 1H), 6.48 (d, |
| | | | | | | | J = 1.8 Hz, 2H), 3.74 (s, 3H) |
| Cpd | LC-17 | 1.53 | | 397 | 400 | DMSO- | 12.21 (s, 1H), 10.91 (s, 1H), 7.94 (dd, J = 10.3, 5.9 Hz, |
| 661 | | | | | | d6 | 1H), 7.64 (m, 1H), 7.46 (m, 1H), 7.21 (d, J = 1.1 Hz, |
| | | | | | | | 1H), 6.71 (m, 1H), 4.07 (d, J = 1.0 Hz, 3H) |
| Cpd | LC-5 | 2.66 | 439 | | 400 | DMSO- | 12.24 (s, 1H), 10.39 (s, 1H), 8.31-8.29 (m, 1H), 7.88- |
| 662 | | | | | | d6 | 7.81 (m, 2H), 7.58-7.53 (m, 2H), 6.96 (s, 1H), 4.80-4.78 |
| | | | | | | | (m, 1H), 4.68-4.67 (m, 1H), 4.40-4.38 (m, 1H), 4.33- |
| | | | | | | | 4.31 (m, 1H) |
| Cpd | LC-5 | 3.10 | 466 | | 400 | DMSO- | 12.47 (s, 1H), 10.98 (s, 1H), 8.92 (s, 1H), 7.89-7.81 (m, |
| 663 | | | | | | d6 | 3H), 7.61 (d, 1H), 7.48 (t, 2H), 7.19 (br s, 1H) |
| Cpd | LC-5 | 2.99 | 439 | | 400 | DMSO- | 12.07 (s, 1H), 10.05 (s, 1H), 7.57 (s, 1H), 7.50 (s, 1H), |
| 664 | | | | | | d6 | 7.41-7.01 (m, 4H), 6.65 (bs, 1H) |
| Cpd | LC-5 | 2.57 | 403 | | 400 | DMSO- | 12.57 (s, 1H), 10.39 (s, 1H), 7.89-7.84 (m, 1H), 7.82 (d, |
| 665 | | | | | | d6 | 1H), 7.68-7.67 (m, 1H), 7.38 (s, 1H), 6.92 (s, 1H), 4.80- |
| | | | | | | | 4.79 (m, 1H), 4.68-4.67 (m, 1H), 4.40-4.39 (m, 1H), |
| | | | | | | | 4.33-4.32 (m, 1H) |
| Cpd | LC-28 | 1.27 | | 441 | 400 | DMSO- | 12.06 (s, 1H), 10.07 (s, 1H), 7.44-7.30 (m, 3H), 7.30- |
| 666 | | | | | | d6 | 7.01 (m, 2H), 6.64 (m, 1H), 4.07 (s, 3H) |
| Cpd | LC-35 | 1.53 | 433 | | 400 | DMSO- | 13.06 (s, 1H), 11.01 (s, 1H), 8.46 (d, J = 1.5 Hz, 1H), |
| 667 | | | | | | d6 | 7.95 (dd, J = 10.3, 5.9 Hz, 1H), 7.80 (dd, J = 3.2, 1.7 |
| | | | | | | | Hz, 1H), 7.51 (dd, J = 10.9, 6.4 Hz, 1H), 7.22 (m, 1H) |
| Cpd | LC-35 | 1.57 | | 458 | 400 | DMSO- | 12.96 (s, 1H), 10.20 (s, 1H), 8.11 (dd, J = 8.0, 1.3 Hz, |
| 668 | | | | | | d6 | 1H), 7.97 (d, J = 8.1 Hz, 1H), 7.56-7.00 (m, 7H) |
| Cpd | LC-27 | 1.24 | 425 | | 400 | DMSO- | 12.10 (s, 1H), 10.71 (s, 1H), 7.75-7.70 (m, 2H), 7.66 (s, |
| 669 | | | | | | d6 | 1H), 7.50-7.45 (m, 1H), 6.99 (s, 1H), 6.70 (s, 1H) |

### Part B

### 1. GPR17 Recombinant Cell Lines

1.1 HEK-293 hGPR17 (Gα-q Assay)

HEK-293 cells stably expressing the human GPR17 receptor (HEK-293 hGPR17) developed by Axxam (Bresso, Milan, Italy) were cultured at 37° C. in a humidified atmosphere of 5% CO₂. Cells were grown in EMEM supplemented with FBS (10%), Penicillin/Streptomycin (1%), Ultraglutamine I (2 mM), puromycin (0.6 μg/mL), G418 (0.4 mg/mL), zeocin (50 μg/mL). This cell line was used to test the compound antagonistic activity by monitoring the Gα-q based signaling. Signaling via Gα-q leads to mobilization of calcium from internal stores. Elevated intracellular calcium levels can then be measured with calcium-sensitive fluorescent dyes (e.g., Fluo 8-No Wash Dye).

1.2 HEK-293 Suchi5 hGPR17 (Gα-i/g Assay)

HEK-293 cells stably expressing the human GPR17 receptor and a Gα-i/q chimera (HEK-293 Suchi5 hGPR17) developed by Axxam (Bresso, Milan, Italy) were cultured at 37° C. in a humidified atmosphere of 5% CO₂. Cells were grown in EMEM supplemented with FBS (10%), Penicillin/Streptomycin (1%), Ultraglutamine 1 (2 mM), blasticidin (4 μg/mL), G418 (0.4 mg/mL). This cell line was used to test the compound antagonistic activity by monitoring the native Gα-i signaling (that leads to modulation of cAMP levels) switched to Gα-q pathway thanks to overexpression of a Gα-i/q chimera (Suchi5). Elevated intracellular calcium levels can then be measured with calcium-sensitive fluorescent dyes (e.g., Fluo-8 No Wash Dye).

### 2. Functional In Vitro GPR17 Assay

### 2.1 Calcium Mobilization Functional Assay

GPR17 activation leads to both an increase in intracellular calcium (via Gα-q) and a decrease in cAMP levels (via Gα-i), implicating that both of these pathways play a role for in vivo function.

Experiments were performed using the below cell lines, as described in part 1:
HEK-293 hGPR17 (used to study the compounds acting via Gα-q pathway)
HEK-293 Suchi5 hGPR17 (used to study the compounds acting via Gα-i pathway, where the Gα-i signaling is switched to a Gα-q signaling thanks to the Gα-i/q chimera Suchi5)

GPR17 activation was able to induce an endoplasmic reticulum calcium (Ca²⁺) store release in cytosol which could be measured using the fluorescent Ca²⁺ sensitive dye Fluo-8 No Wash Dye as readout. Any antagonistic compound activity was detected as an inhibition of the fluorescent signal generated by GPR17 activation.

### 2.2 Description of Ca²⁺ Assay

HEK-293 hGPR17 and HEK-293 Suchi5 hGPR17 were seeded at a density of 15,000 cells/well into poly-D-lysine coated black 384-well plates with clear bottom in complete medium. Cells were incubated overnight at 37° C. in a humidified atmosphere of 5% CO₂. Twenty-four hours after seeding, the culture medium was carefully removed manually and the cells were loaded for 60 minutes at room temperature with the Ca²⁺ sensitive Fluo-8 No Wash Dye, according to manufacturer's instructions. Cells were then assayed using a fluorometric imaging plate reader (FLIPR^{TETRA}). Fluorescence (excitation: 470-495 nm; emission: 515-575 nm) was recorded during the experiment. After recording of baseline fluorescence (approx. 10 see), both test compounds (typically 10⁻⁹ M to 10⁻⁶ M) and controls (MDL29,951, a GPR17 agonist, and Pranlukast, a GPR17 antagonist) diluted in assay buffer were injected upon the cells at the FLIPR^{TETRA} and the kinetic response was monitored over a period of 2 minutes. After twenty minutes, a second injection of MDL29,951 at ˜EC₈₀ (500 nM for HEK-293 hGPR17 and 2 nM for HEK-293 Suchi5 hGPR17) in assay buffer was performed at the FLIPR^{TETRA} and the signal of the emitted fluorescence was recorded for additional 2 minutes. All the compound injections and incubations were performed in duplicate. For data quality and data analysis the Screener® 16.0.6 (Genedata) software was used. Target inhibition was expressed as a percentage of activity, with −100% activity being a results in which the kinetic response value of the test wells reached a level identical of the one of the Inhibitor Controls (injection of reference inhibitor Pranlukast at IC₁₀₀ followed by the injection of the reference agonist at EC₈₀) and 0% activity being a result in which the Response Value of the test wells reaches a level identical to the one of the Neutral Controls (injection of assay buffer followed by the injection of the reference agonist at EC₈₀).

The compounds listed below showed 1050 on hGPR17 below 0.5 μM down to low nM activity:
Cpd 002; Cpd 003; Cpd 004; Cpd 005; Cpd 006; Cpd 007; Cpd 010; Cpd 014; Cpd 015; Cpd 016; Cpd 017; Cpd 023; Cpd 025; Cpd 026; Cpd 028; Cpd 033; Cpd 034; Cpd 035; Cpd 038; Cpd 039; Cpd 040; Cpd 041; Cpd 042; Cpd 044; Cpd 046; Cpd 050; Cpd 061; Cpd 063; Cpd 065; Cpd 068; Cpd 070; Cpd 071; Cpd 072; Cpd 073; Cpd 074; Cpd 075; Cpd 076; Cpd 082; Cpd 083; Cpd 084; Cpd 088; Cpd 089; Cpd 090; Cpd 091; Cpd 102; Cpd 103; Cpd 116; Cpd 119; Cpd 124; Cpd 148; Cpd 155; Cpd 171; Cpd 172; Cpd 173; Cpd 177; Cpd 191; Cpd 202; Cpd 204; Cpd 205; Cpd 206; Cpd 210; Cpd 215; Cpd 224; Cpd 226; Cpd 228; Cpd 230; Cpd 232; Cpd 234; Cpd 238; Cpd 244; Cpd 246; Cpd 256; Cpd 257; Cpd 258; Cpd 260; Cpd 267; Cpd 269; Cpd 271; Cpd 272; Cpd 273; Cpd 275; Cpd 276; Cpd 278; Cpd 281; Cpd 285; Cpd 289; Cpd 290; Cpd 302; Cpd 309; Cpd 318; Cpd 326; Cpd 331; Cpd 333; Cpd 339; Cpd 343; Cpd 346; Cpd 348; Cpd 349; Cpd 350; Cpd 353; Cpd 357; Cpd 358; Cpd 360; Cpd 367; Cpd 372; Cpd 374; Cpd 380; Cpd 391; Cpd 392; Cpd 393; Cpd 394; Cpd 396; Cpd 401; Cpd 403; Cpd 404; Cpd 405; Cpd 406; Cpd 407; Cpd 409; Cpd 413; Cpd 416; Cpd 418; Cpd 420; Cpd 422; Cpd 430; Cpd 436; Cpd 439; Cpd 440; Cpd 441; Cpd 442; Cpd 443; Cpd 445; Cpd 448; Cpd 449; Cpd 452; Cpd 453; Cpd 455; Cpd 456; Cpd 459; Cpd 462; Cpd 474; Cpd 475; Cpd 476; Cpd 477; Cpd 478; Cpd 479; Cpd 480; Cpd 484; Cpd 489; Cpd 494; Cpd 497; Cpd 502; Cpd 504; Cpd 508; Cpd 509; Cpd 510; Cpd 511; Cpd 512; Cpd 515; Cpd 516; Cpd 522; Cpd 523; Cpd 527; Cpd 528; Cpd 529; Cpd 530; Cpd 531; Cpd 535; Cpd 536; Cpd 539; Cpd 541; Cpd 542; Cpd 543; Cpd 544; Cpd 545; Cpd 546; Cpd 548; Cpd 549; Cpd 550; Cpd 551; Cpd 554; Cpd 555; Cpd 556; Cpd 557; Cpd 559; Cpd 562; Cpd 563; Cpd 567; Cpd 569; Cpd 570; Cpd 572; Cpd 575; Cpd 577; Cpd 578; Cpd 584; Cpd 585; Cpd 588; Cpd 594; Cpd 597; Cpd 598; Cpd 599; Cpd 602; Cpd 610; Cpd 612; Cpd 615; Cpd 619; Cpd 623; Cpd 626; Cpd 628; Cpd 629; Cpd 632; Cpd 633; Cpd 634; Cpd 635; Cpd 636; Cpd 638; Cpd 639; Cpd 640; Cpd 641; Cpd 642; Cpd 643; Cpd 645; Cpd 646; Cpd 647; Cpd 648; Cpd 649; Cpd 650; Cpd 651; Cpd 652; Cpd 653; Cpd 654; Cpd 655; Cpd 659; Cpd 660; Cpd 661; Cpd 662; Cpd 665; Cpd 666; Cpd 669.

The compounds listed below showed IC₅₀ on hGPR17 between 0.5 and 5 μM activity:
Cpd 001; Cpd 008; Cpd 009; Cpd 011; Cpd 018; Cpd 019; Cpd 020; Cpd 021; Cpd 027; Cpd 030; Cpd 031; Cpd 036; Cpd 043; Cpd 045; Cpd 047; Cpd 048; Cpd 049; Cpd 051; Cpd 052; Cpd 053; Cpd 055; Cpd 057; Cpd 058; Cpd 059; Cpd 060; Cpd 062; Cpd 064; Cpd 066; Cpd 069; Cpd 077; Cpd 079; Cpd 080; Cpd 081; Cpd 085; Cpd 086; Cpd 087; Cpd 093; Cpd 095; Cpd 096; Cpd 097; Cpd 098; Cpd 099; Cpd 100; Cpd 101; Cpd 104; Cpd 105; Cpd 106; Cpd 107; Cpd 108; Cpd 110; Cpd 111; Cpd 112; Cpd 113; Cpd 114; Cpd 117; Cpd 118; Cpd 123; Cpd 126; Cpd 129; Cpd 131; Cpd 132; Cpd 134; Cpd 135; Cpd 136; Cpd 137; Cpd 139; Cpd 140; Cpd 141; Cpd 142; Cpd 143; Cpd 145; Cpd 147; Cpd 149; Cpd 150; Cpd 154; Cpd 156; Cpd 157; Cpd 158; Cpd 162; Cpd 164; Cpd 165; Cpd 166; Cpd 167; Cpd 168; Cpd 170; Cpd 175; Cpd 176; Cpd 178; Cpd 179; Cpd 180; Cpd 181; Cpd 182; Cpd 183; Cpd 184; Cpd 185; Cpd 186; Cpd 187; Cpd 188; Cpd 189; Cpd 190; Cpd 193; Cpd 194; Cpd 195; Cpd 198; Cpd 203; Cpd 208; Cpd 211; Cpd 214; Cpd 216; Cpd 218; Cpd 219; Cpd 220; Cpd 221; Cpd 227; Cpd 229; Cpd 231; Cpd 233; Cpd 235; Cpd 237; Cpd 239; Cpd 240; Cpd 247; Cpd 248; Cpd 249; Cpd 250; Cpd 252; Cpd 253; Cpd 254; Cpd 255; Cpd 259; Cpd 261; Cpd 262; Cpd 263; Cpd 264; Cpd 265; Cpd 268; Cpd 270; Cpd 274; Cpd 277; Cpd 279; Cpd 280; Cpd 284; Cpd 286; Cpd 287; Cpd 288; Cpd 291; Cpd 292; Cpd 296; Cpd 298; Cpd 299; Cpd 300; Cpd 301; Cpd 303; Cpd 304; Cpd 305; Cpd 306; Cpd 307; Cpd 308; Cpd 310; Cpd 311; Cpd 312; Cpd 313; Cpd 314; Cpd 315; Cpd 316; Cpd 317; Cpd 319; Cpd 320; Cpd 321; Cpd 322; Cpd 323; Cpd 325; Cpd 327; Cpd 328; Cpd 329; Cpd 332; Cpd 334; Cpd 336; Cpd 337; Cpd 338; Cpd 340; Cpd 341; Cpd 342; Cpd 344; Cpd 345; Cpd 347; Cpd 351; Cpd 352; Cpd 354; Cpd 355; Cpd 356; Cpd 359; Cpd 361; Cpd 363; Cpd 364; Cpd 365; Cpd 366; Cpd 368; Cpd 371; Cpd 373; Cpd 378; Cpd 379; Cpd 382; Cpd 384; Cpd 388; Cpd 397; Cpd 398; Cpd 399; Cpd 400; Cpd 408; Cpd 410; Cpd 411; Cpd 412; Cpd 414; Cpd 415; Cpd 417; Cpd 419; Cpd 421; Cpd 423; Cpd 424; Cpd 425; Cpd 426; Cpd 427; Cpd 428; Cpd 429; Cpd 432; Cpd 434; Cpd 435; Cpd 444; Cpd 446; Cpd 447; Cpd 450; Cpd 451; Cpd 454; Cpd 457; Cpd 458; Cpd 460; Cpd 463; Cpd 464; Cpd 465; Cpd 466; Cpd 468; Cpd 469; Cpd 470; Cpd 471; Cpd 472; Cpd 473; Cpd 481; Cpd 482; Cpd 483; Cpd 485; Cpd 486; Cpd 488; Cpd 492; Cpd 495; Cpd 496; Cpd 498; Cpd 501; Cpd 503; Cpd 506; Cpd 507; Cpd 513; Cpd 514; Cpd 517; Cpd 518; Cpd 519; Cpd 524; Cpd 525; Cpd 526; Cpd 532; Cpd 533; Cpd 534; Cpd 537; Cpd 538; Cpd 540; Cpd 547; Cpd 552; Cpd 553; Cpd 558; Cpd 561; Cpd 564; Cpd 565; Cpd 566; Cpd 568; Cpd 573; Cpd 574; Cpd 576; Cpd 580; Cpd 582; Cpd 586; Cpd 587; Cpd 589; Cpd 592; Cpd 593; Cpd 595; Cpd 596; Cpd 601; Cpd 604; Cpd 605; Cpd 606; Cpd 609; Cpd 611; Cpd 613; Cpd 616; Cpd 618; Cpd 620; Cpd 621; Cpd 622; Cpd 624; Cpd 627; Cpd 630; Cpd 631; Cpd 637; Cpd 656; Cpd 657; Cpd 658; Cpd 663; Cpd 664; Cpd 668.

The compounds listed below showed 1050 on hGPR17 between 5 and 50 μM activity:
Cpd 012; Cpd 013; Cpd 022; Cpd 024; Cpd 029; Cpd 032; Cpd 037; Cpd 054; Cpd 056; Cpd 067; Cpd 078; Cpd 094; Cpd 109; Cpd 115; Cpd 120; Cpd 122; Cpd 125; Cpd 127; Cpd 128; Cpd 130; Cpd 133; Cpd 138; Cpd 144; Cpd 146; Cpd 151; Cpd 152; Cpd 153; Cpd 159; Cpd 160; Cpd 161; Cpd 163; Cpd 169; Cpd 174; Cpd 192; Cpd 196; Cpd 197; Cpd 199; Cpd 200; Cpd 201; Cpd 207; Cpd 209; Cpd 217; Cpd 222; Cpd 223; Cpd 236; Cpd 241; Cpd 242; Cpd 243; Cpd 245; Cpd 251; Cpd 266; Cpd 282; Cpd 283; Cpd 293; Cpd 294; Cpd 295; Cpd 297; Cpd 324; Cpd 330; Cpd 335; Cpd 362; Cpd 369; Cpd 370; Cpd 375; Cpd 381; Cpd 383; Cpd 385; Cpd 386; Cpd 387; Cpd 389; Cpd 402; Cpd 431; Cpd 433; Cpd 437; Cpd 438; Cpd 461; Cpd 490; Cpd 491; Cpd 493; Cpd 499; Cpd 500; Cpd 505; Cpd 520; Cpd 521; Cpd 560; Cpd 571; Cpd 581; Cpd 590; Cpd 591; Cpd 600; Cpd 603; Cpd 607; Cpd 608; Cpd 614; Cpd 617; Cpd 625; Cpd 644; Cpd 667.

1. A compound of formula (I), or a tautomer, a stereoisomer, a hydrate, a solvate, a polymorph, a prodrug, an isotope, or a co-crystal thereof, or a pharmaceutically acceptable salt thereof, wherein 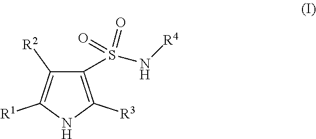
R¹ is selected from the group comprising aryl, heteroaryl, cycloalkyl, cycloalkenyl, cycloalkynyl, heterocyclyl, and A¹-X¹—; and R² is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl;
wherein each of said aryl, heteroaryl, cycloalkyl, cycloalkenyl, cycloalkynyl, heterocyclyl, X¹ and A¹ of R¹ can be unsubstituted or substituted with one or more Z¹;
X¹ is —Y^{1b}—Y^{1a}—Y^{1c}—, wherein Y^{1a} is a single bond, double bond or triple bond or is selected from the group comprising —CR^{1a}═CR^{1a}—, —C≡C—, —CO—, —O—, —CS—, —S—, —SO₂—, —SO—, —SO(NH)—, —CONR^{1b}—, —NR^{1b}CO—, —SO₂NR^{1b}—, —NR^{1b}SO₂—, —S(O)—NR^{1b}—, and —NR^{1b}—;
each of Y^{1b} and Y^{1c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{1a}; wherein when Y^{1a} is a single bond, double bond, or triple bond, at least one of Y^{1b} and Y^{1c} is not a single bond;
each R^{1a} is independently selected from the group comprising hydrogen, oxo, thioxo, halo, hydroxy, haloalkyl, alkoxy, alkoxyalkyl, haloalkoxy, haloalkoxyalkyl, mono or di(alkyl)amino, mono or di(alkyl)aminoalkyl, and alkyl;
A¹ is selected from the group comprising aryl, heteroaryl, cycloalkyl, cycloalkenyl, cycloalkynyl, and heterocyclyl;
each Z¹ is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, hydroxyalkyl, alkoxyalkyl, cycloalkyloxy, cycloalkylalkoxy, alkoxyalkoxy, carboxyl, alkoxycarbonyl, alkylcarbonyl, arylalkoxy, amino, mono or di(alkyl)amino, aminoalkyl, mono or di(alkyl)aminoalkyl, mono or di(alkyl)aminocarbonyl, heterocyclyl, heteroaryl, heterocyclylalkyl, heteroarylalkyl, arylalkenyl, arylalkynyl, haloalkenyloxy, haloalkynyloxy, hydroxyalkenyl, hydroxyalkynyl, alkenyloxyalkyl, alkynyloxyalkyl, alkoxyalkenyl, alkoxyalkynyl, alkenyloxyalkoxy, alkynyloxyalkoxy, alkenyloxycarbonyl, alkynyloxycarbonyl, alkenylcarbonyl, alkynylcarbonyl, aminoalkenyl, aminoalkynyl, mono or di(alkyl)aminoalkenyl, mono or di(alkyl)aminoalkynyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{1a};
and/or two Z¹ together with the atom(s) to which they are attached can form an aryl, a cycloalkyl, a heteroaryl, or a heterocyclyl; wherein each of said aryl, cycloalkyl, heteroaryl, and heterocyclyl can be unsubstituted or substituted with one or more Z^{1a};
and/or one R^{1a} together with one Z¹ and the atom(s) to which they are attached can form a cycloalkyl, a 4-10 membered saturated or partially saturated heterocyclyl, a 5-10 membered heteroaryl, or an aryl; wherein each of said cycloalkyl, heterocyclyl, heteroaryl or aryl can be unsubstituted or substituted with one or more Z^{1a};
R^{1b} is hydrogen or alkyl, or R^{1b} together with one Z and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a};
each Z^{1a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
or R¹ is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl; and R² is selected from the group comprising aryl, heteroaryl, cycloalkyl, cycloalkenyl, cycloalkynyl, heterocyclyl, and A²-X²—;
wherein each of said aryl, heteroaryl, cycloalkyl, cycloalkenyl, cycloalkynyl, heterocyclyl,
X² and A² of R², can be unsubstituted or substituted with one or more Z²;
X² is —Y^{2b}—Y^{2a}—Y^{2c}—, wherein Y^{2a} is a single bond, double bond or triple bond or is selected from the group comprising —CR^{2a}═CR^{2a}—, —C≡C—, —CO—, —O—, —CS—, —S—, —SO₂—, —SO—, —SO(NH)—, —CONR^{2b}—, —NR^{2b}CO—, —SO₂NR^{2b}—, —NR^{2b}SO₂—, —S(O)—NR^{2b}—, and —NR^{2b}—;
each of Y^{2b} and —Y^{2c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{2a}; wherein when Y^{2a} is a single bond, double bond, or triple bond, at least one of Y^{2b} and Y^{2c} is not a single bond;
each R^{2a} is independently selected from the group comprising hydrogen, oxo, thioxo, halo, hydroxy, haloalkyl, alkoxy, alkoxyalkyl, haloalkoxy, haloalkoxyalkyl, mono or di(alkyl)amino, mono or di(alkyl)aminoalkyl, and alkyl;
A² is selected from the group comprising aryl, heteroaryl, cycloalkyl, cycloalkenyl, cycloalkynyl, and heterocyclyl;
each Z² is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, arylalkenyl, arylalkynyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, haloalkenyloxy, haloalkynyloxy, hydroxyalkyl, hydroxyalkenyl, hydroxyalkynyl, alkoxyalkyl, alkenyloxyalkyl, alkynyloxyalkyl, alkoxyalkenyl, alkoxyalkynyl, cycloalkyloxy, cycloalkylalkoxy, alkoxyalkoxy, alkenyloxyalkoxy, alkynyloxyalkoxy, carboxyl, alkoxycarbonyl, alkenyloxycarbonyl, alkynyloxycarbonyl, alkylcarbonyl, alkenylcarbonyl, alkynylcarbonyl, arylalkoxy, amino, mono or di(alkyl)amino, aminoalkyl, aminoalkenyl, aminoalkynyl, mono or di(alkyl)aminoalkyl, mono or di(alkyl)aminoalkenyl, mono or di(alkyl)aminoalkynyl, mono or di(alkyl)aminocarbonyl, heterocyclyl, heteroaryl, heterocyclylalkyl, heteroarylalkyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, arylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{2a};
and/or two Z² together with the atom(s) to which they are attached can form an aryl, a cycloalkyl, a heteroaryl, or a heterocyclyl; wherein each of said aryl, cycloalkyl, heteroaryl, and heterocyclyl can be unsubstituted or substituted with one or more Z^{2a};
and/or one R^{2a} together with one Z² and the atom(s) to which they are attached can form a cycloalkyl, a 4-10 membered saturated or partially saturated heterocyclyl, a 5-10 membered heteroaryl, or an aryl; wherein each of said cycloalkyl, heterocyclyl, heteroaryl, or aryl can be unsubstituted or substituted with one or more Z^{2a};
R^{2b} is hydrogen or alkyl, or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a};
each Z^{2a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
R³ is selected from the group comprising hydrogen, halo, cyano, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, alkoxyalkyl, mono or di(alkyl)amino, and mono or di(alkyl)aminoalkyl;
R⁴ is aryl, or heteroaryl;
wherein each of said aryl and heteroaryl, is substituted with one or more Z⁴;
each Z⁴ is independently selected from halo, cyano, oxo, nitro, thioxo, or from the group comprising hydroxy, thio, alkyl, alkenyl, alkynyl, cycloalkyl, cycloalkylalkyl, cycloalkenyl, cycloalkynyl, cycloalkenylalkyl, cycloalkynylalkyl, aryl, arylalkyl, arylalkenyl, arylalkynyl, haloalkyl, haloalkenyl, haloalkynyl, cyanoalkyl, alkoxy, alkenyloxy, alkynyloxy, cyanoalkoxy, alkylthio, alkenylthio, alkynylthio, haloalkoxy, haloalkenyloxy, haloalkynyloxy, hydroxyalkyl, hydroxyalkenyl, hydroxyalkynyl, alkoxyalkyl, alkenyloxyalkyl, alkynyloxyalkyl, alkoxyalkenyl, alkoxyalkynyl, cycloalkyloxy, cycloalkylalkoxy, alkoxyalkoxy, alkenyloxyalkoxy, alkynyloxyalkoxy, carboxyl, alkoxycarbonyl, alkenyloxycarbonyl, alkynyloxycarbonyl, alkylcarbonyl, alkenylcarbonyl, alkynylcarbonyl, arylalkoxy, amino, mono or di(alkyl)amino, aminoalkyl, aminoalkenyl, aminoalkynyl, mono or di(alkyl)aminoalkyl, mono or di(alkyl)aminoalkenyl, mono or di(alkyl)aminoalkynyl, mono or di(alkyl)aminocarbonyl, heterocyclyl, heteroaryl, heterocyclylalkyl, heteroarylalkyl, heterocyclylalkenyl, heterocyclylalkynyl, heteroarylalkenyl, heteroarylalkynyl, aryloxy, aryloxyalkyl, aryloxyalkenyl, aryloxyalkynyl, arylthio, haloalkythio, cycloalkylthio, alkylsulfinyl, alkylsulfonyl, cycloalkylsulfinyl, cycloalkylsulfonyl, arylsulfinyl, arylsulfonyl, mono or di(alkyl)aminosulfonyl, mono or di(alkyl)aminosulfinyl, alkoxycarbonylamino, alkenyloxycarbonylamino, alkynyloxycarbonylamino, alkylcarbonylamino, alkenylcarbonylamino, alkynylcarbonylamino, cycloalkylcarbonylamino, arylcarbonylamino, cycloalkylcarbonyl, arylcarbonyl, mono or di(alkyl)aminocarbonyl, alkylcarbonyloxy, alkenylcarbonyloxy, alkynylcarbonyloxy, arylcarbonyloxy, sulfonyl, sulfinyl, mono or di(alkyl)aminoalkylamino, mono or di(alkyl)aminoalkoxy, arylamino, arylaminoalkyl, alkylcarbonyloxyalkyl, alkenylcarbonyloxyalkyl, alkynylcarbonyloxyalkyl, arylcarbonyloxy, arylcarbonyloxyalkyl, arylaminocarbonyl, heterocyclyloxy, heteroaryloxy, heteroarylthio, heteroaryloxyalkyl, heteroaryloxyalkenyl, heteroaryloxyalkynyl, heteroarylsulfinyl, heteroarylsulfonyl, heteroarylamino, heteroarylaminoalkyl, heteroarylcarbonylamino, heteroarylcarbonyl, heteroarylcarbonyloxy, heteroarylcarbonyloxyalkyl, and heteroarylaminocarbonyl; each of said group can be unsubstituted or substituted with one or more Z^{4a};
and/or two Z⁴ together with the atom(s) to which they are attached can form an aryl, a cycloalkyl, a heteroaryl, or a heterocyclyl, wherein each of said aryl, heteroaryl, cycloalkyl, and heterocyclyl can be unsubstituted or substituted with one or more Z^{4a};
each Z^{4a} is independently selected from the group comprising halo, cyano, hydroxyl, alkyl, alkenyl, alkynyl, haloalkyl, haloalkenyl, haloalkynyl, alkoxy, alkenyloxy, alkynyloxy, alkylthio, alkenylthio, alkynylthio, haloalkoxy, hydroxyalkyl, alkoxyalkyl, cycloalkyl, cycloalkenyl, cycloalkynyl, cycloalkyloxy, aryl, arylalkyl, amino, mono or di(alkyl)amino, mono or di(alkyl)aminoalkyl, and oxo;
with the proviso that
when R¹ is A¹-X¹—, X¹ is —CO—, and A¹ is heterocyclyl, then A¹ is not attached to X¹ via an N ring atom of said heterocyclyl;
when R¹ is a heteroaryl, R¹ is not oxadiazolyl;
when R² is A²-X²—, X² is —CO—, and A² is heterocyclyl, then A² is not attached to X² via an N ring atom of said heterocyclyl; and
when R² is a heteroaryl, R² is not oxadiazolyl;
with the proviso that said compound is not
N,4-bis(4-methylphenyl)-1H-pyrrole-3-sulfonamide; (CAS no 1427286-05-2),
N,4-bis(4-chlorophenyl)-1H-pyrrole-3-sulfonamide (CAS no 1427286-06-3).

2. The compound according to claim 1, wherein
R¹ is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl and A¹-X¹—, preferably R¹ is selected from the group comprising C₆₋₁₀aryl, 5-10 membered heteroaryl, C₃₋₁₀cycloalkyl, C₅₋₁₀cycloalkenyl, and A¹-X¹—;
wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X¹ and A¹ of R¹, can be unsubstituted or substituted with one or more Z¹; and
R² is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; preferably R² is selected from the group comprising hydrogen, halo, cyano, C₁₋₆alkyl, haloC₁₋₆alkyl, C₁₋₆alkoxy, C₁₋₆alkylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl.

3. The compound according to claim 1, wherein
X¹ is —Y^{1b}—Y^{1a}—Y^{1c}—, wherein Y^{1a} is a single bond, double bond or triple bond or is selected from the group comprising —CR^{1a}═CR^{1a}—, —C≡C—, —CO—, —O—, —CS—, —S—, —SO₂—, —SO—, —SO(NH)—, CONR^{1b}—, —NR^{1b}CO—, —SO₂NR^{1b}—, —NR^{1b}SO₂—, —S(O)—NR^{1b}—, and —NR^{1b}—; preferably X¹ is selected from the group comprising —C(R^{1a})₂—, —CR^{1a}—CR^{1a}—, —C≡C—, —CO—, —O—, —CS—, —S—, —SO₂—, —SO—, —SO(NH)—, —CONR^{1b}—, —NR^{1b}CO—, —SO₂NR^{1b}—, —NR^{1b}SO₂—, —S(O)—NR^{1b}—, and —NR^{1b}—;
each of Y^{1b} and Y^{1c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{1a};
wherein when Y^{1a} is a single bond, double bond or triple bond, at least one of Y^{1b} and Y^{1c} is not a single bond;
each R^{1a} is independently selected from the group comprising hydrogen, oxo, thioxo, halo, hydroxy, haloC₁₋₆alkyl, C₁₋₆alkoxy, C₁₋₆alkoxyC₁₋₆alkyl, haloC₁₋₆alkoxy, haloC₁₋₆alkoxyC₁₋₆alkyl, mono or di(C₁₋₆alkyl)amino, mono or di(C₁₋₆alkyl)aminoC₁₋₆alkyl, and C₁₋₆alkyl; preferably each R^{1a} is independently selected from the group comprising hydrogen, halo, hydroxy, haloC₁₋₆alkyl, C₁₋₆alkoxy, haloC₁₋₆alkoxy, and C₁₋₆alkyl;
A¹ is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl; preferably A¹ is selected from the group comprising C₆₋₁₀aryl, 5-10 membered heteroaryl, C₃₋₁₀cycloalkyl, and C₅₋₁₀cycloalkenyl;
and/or one R^{1a} together with one Z¹ and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{1a};
R^{1b} is hydrogen or C₁₋₆alkyl; preferably each R^{1b} is independently selected from hydrogen, or C₁₋₄alkyl; or R^{1b} together with one Z¹ and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{1a}.

4. The compound according to claim 1, wherein
R² is selected from the group comprising C₆₋₁₀aryl, 5-10 membered heteroaryl, C₃₋₁₀cycloalkyl, C₅₋₁₀cycloalkenyl, 3-10 membered saturated or partially saturated heterocyclyl and A²-X²—; preferably R² is selected from the group comprising C₆₋₁₀aryl, 5-10 membered heteroaryl, C₃₋₁₀cycloalkyl, C₅₋₁₀cycloalkenyl, and A²-X²—;
wherein each of said C₆₋₁₀aryl, 5-10 membered heteroaryl, C₃₋₁₀cycloalkyl, C₅₋₁₀cycloalkenyl, 3-10 membered saturated or partially saturated heterocyclyl, X² and A² of R², can be unsubstituted or substituted with one or more Z²; and
R¹ is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; preferably R¹ is selected from the group comprising hydrogen, halo, cyano, C₁₋₆alkyl, haloC₁₋₆alkyl, C₁₋₆alkoxy, C₁₋₆alkylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl.

5. The compound according to claim 1, wherein
X² is —Y^{2b}'Y^{2a}—Y^{2c}—, wherein Y^{2a} is a single bond, double bond or triple bond or is selected from the group comprising —CR^{2a}═CR^{2a}—, —C≡C—, —CO—, —O—, —CS—, —S—, —SO₂—, —SO—, —SO(NH)—, —CONR^{2b}—, —NR^{2b}CO—, —SO₂NR^{2b}—, —NR^{2b}SO₂—, —S(O)—NR^{2b}—, and —NR^{2b}—; preferably X² is selected from the group comprising —C(R^{2a})₂—, —CR^{2a}═CR^{2a}—, —C≡C—, —CO—, —O—, —CS—, —S—, —SO₂—, —SO—, —SO(NH)—, —CONR^{2b}—, —NR^{2b}CO—, —SO₂NR^{2b}—, —NR^{2b}SO₂—, —S(O)—NR^{2b}—, and —NR^{2b}—;
each of Y^{2b} and Y^{2c} is independently selected from the group comprising a single bond, or C₁₋₃alkylene, C₂₋₃alkenylene, C₂₋₃alkynylene; wherein each of said C₁₋₃alkylene, C₂₋₃alkenylene, C₂₋₃alkynylene can be unsubstituted or substituted with one or more R^{2a}; wherein when Y^{2a} is a single bond, double bond, or triple bond, at least one of Y^{2b} and Y^{2c} is not a single bond;
each R^{2a} is independently selected from the group comprising hydrogen, oxo, thioxo, halo, hydroxy, haloC₁₋₆alkyl, C₁₋₆alkoxy, C₁₋₆alkoxyC₁₋₆alkyl, haloC₁₋₆alkoxy, haloC₁₋₆alkoxyC₁₋₆alkyl, mono or di(C₁₋₆alkyl)amino, mono or di(C₁₋₆alkyl)aminoC₁₋₆alkyl, and C₁₋₆alkyl; preferably each R^{2a} is independently selected from the group comprising hydrogen, halo, hydroxy, haloC₁₋₆alkyl, C₁₋₆alkoxy, haloC₁₋₆alkoxy, and C₁₋₆alkyl;
A² is selected from the group comprising C₆₋₁₀aryl, 5-10 membered heteroaryl, C₃₋₁₀cycloalkyl, C₅₋₁₀cycloalkenyl, and 3-10 membered saturated or partially saturated heterocyclyl; preferably A² is selected from the group comprising C₆₋₁₀aryl, 5-10 membered heteroaryl, C₃₋₁₀cycloalkyl, and C₅₋₁₀cycloalkenyl;
and/or one R^{2a} together with one Z² and the atom(s) to which they are attached can form a C₄₋₁₀cycloalkyl, or a 4-10 membered saturated, or partially saturated heterocyclyl, or a 5-10 membered heteroaryl; wherein each of said C₄₋₁₀cycloalkyl, heterocyclyl or heteroaryl can be unsubstituted or substituted with one or more Z^{2a};
R^{2b} is hydrogen or C₁₋₆alkyl, preferably each R^{2b} is independently selected from hydrogen, or C₁₋₄alkyl; or R^{2b} together with one Z² and the atom(s) to which they are attached can form a 4-10 membered saturated, or partially saturated heterocyclyl or a 5-10 membered heteroaryl; wherein each of said heterocyclyl or heteroaryl can be unsubstituted or substituted with one or more Z^{2a}.

6. The compound according to claim 1, wherein
R³ is selected from the group comprising hydrogen, halo, cyano, C₁₋₆alkyl, C₂₋₆alkenyl, haloC₁₋₆alkyl, haloC₂₋₆alkenyl, C₁₋₆alkoxy, C₂₋₆alkenyloxy, C₁₋₆alkylthio, C₂₋₆alkenylthio, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl; preferably R³ is selected from the group comprising hydrogen, halo, cyano, C₁₋₆alkyl, haloC₁₋₆alkyl, C₁₋₆alkoxy, haloC₁₋₆alkoxy, C₁₋₆alkoxyC₁₋₆alkyl, mono or di(C₁₋₆alkyl)amino, and mono or di(C₁₋₆alkyl)aminoC₁₋₆alkyl.

7. The compound according to claim 1, wherein
R⁴ is C₆₋₁₀aryl, or 5-10 membered heteroaryl; preferably R⁴ is C₆₋₁₀aryl, or 5-8 membered heteroaryl;
wherein each of said C₆₋₁₀aryl and 5-10 membered heteroaryl, is substituted with one or more Z⁴; preferably wherein each of said C₆₋₁₀aryl and 5-10 membered heteroaryl, is substituted with two or more Z⁴.

8. The compound according to claim 1, having structural formula (II) 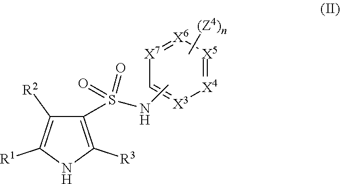
wherein each of X³, X⁴, X⁵, X⁶, and X⁷ is independently selected from CH, or N; provided that no more three X³, X⁴, X⁵, X⁶, and X⁷ are N; n is an integer selected from 1, 2, 3, or 4;
and R¹, R², R³ and Z⁴ have the same meaning as in claim 1.

9. The compound according to claim 1, having structural formula (IX), (X), or (XI), 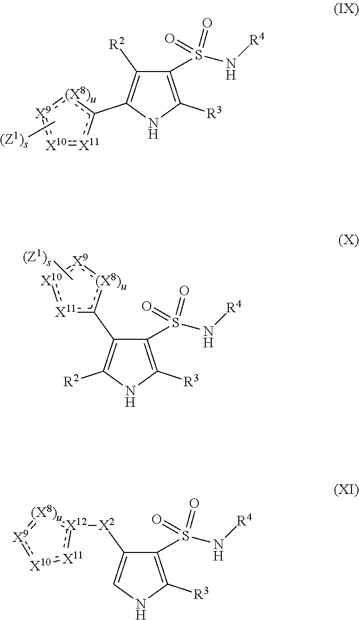
wherein each of X⁸, X⁹, X¹⁰, X¹¹, and X¹² is independently selected from CH, N, O, or S; u is an integer selected from 0, 1, 2 or 3; s is an integer selected from 0, 1, 2, 3, or 4; [symbol] is an optional double bond,
and R⁴, R¹, R², R³ and Z¹ have the same meaning as in claim 1.

10. The compound according to claim 1, wherein said compound is selected from the group comprising the compounds listed in Table A.

11. A pharmaceutical composition comprising a compound according to claim 1, and a pharmaceutical acceptable carrier.

12. A compound according to claim 1 for use as a medicine.

13. A compound according to claim 1 for use in the prevention and/or treatment of GPR17 mediated disorders.

14. A compound according to claim 1 for use in the prevention or treatment of a disorder or syndrome selected from a myelination disorder and a disorder or syndrome associated with brain tissue damage.

15. A compound for use according to claim 13, wherein the syndrome or disorder is selected from the group of Multiple Sclerosis (MS) including all its various subforms including clinically isolated syndrome (CIS); optic neuropathies including acute optic neuritis, chronic relapsing inflammatory optic neuritis, neuromyelitis optica (NMO, Devic's disease); acute disseminated encephalomyelitis, acute hemorrhagic leucoencephalitis (AHL); periventricular leukomalacia; demyelination due to autoimmune diseases including anti-MAG peripheral neuropathy and anti-MOG associated spectrum; genetic diseases with white matter pathologies including but not restricted to Sjogren's syndrome, systemic lupus erythematosus, Gaucher's disease, Niemann-Pick disease; leukodystrophies and genetic leukoencephalopathies and adrenoleukodystrophies; demyelination due to viral or bacterial infections; demyelination due to traumatic brain tissue damage and nerve injury; demyelination in response to hypoxia, stroke or ischemia or other cardiovascular diseases; demyelination due to exposure to carbon dioxide, cyanide, vitamin deficiencies or other CNS toxins; central pontine and extrapontine myelinolysis; Schilder's disease; Balo concentric sclerosis; perinatal encephalopathy; neurodegenerative diseases including amyotrophic lateral sclerosis (ALS), Alzheimer's disease (AD), multiple system atrophy, Parkinson's Disease, Niemann-Pick disease, spinocerebellar ataxia (SCA) and Huntington's Disease (HD); psychiatric disorders such as schizophrenia, bipolar disorder, depression and major depressive disorders; and peripheral myelination diseases including acute and chronic peripheral demyelinating neuropathies, Dejerine-Sottas syndrome or Charcot-Marie Tooth disease.

16. A compound for use according to claim 14, wherein the syndrome or disorder is selected from the group of multiple sclerosis (MS) including its various subforms, optic neuritis, neuromyelitis optica (Devic's disease), chronic relapsing inflammatory optic neuritis, acute disseminated encephalomyelitis, acute hemorrhagic leucoencephalitis (AHL), periventricular leukomalacia, demyelination due to viral or bacterial infections, central pontine and extrapontine myelinolysis, demyelination due to traumatic brain tissue damage, demyelination in response to hypoxia, stroke or ischemia or other cardiovascular diseases, demyelination due to exposure to carbon dioxide, cyanide, or other CNS toxins, Schilder's disease, Balo concentric sclerosis, perinatal encephalopathy, neurodegenerative diseases including amyotrophic lateral sclerosis (ALS), Alzheimer's disease (AD), multiple system atrophy, Parkinson's Disease, spinocerebellar ataxia (SCA) and Huntington's Disease, psychiatric disorders such as schizophrenia and bipolar disorder and peripheral myelination diseases including leukodystrophies, peripheral neuropathies, Dejerine-Sottas syndrome or Charcot-Marie-Tooth disease.
